(19) United States
(12) Patent Application Publication
(10) Pub. No.: US 2024/0382482 A1
(43) Pub. Date: Nov. 21, 2024

(54) PLASMA KALLIKREIN INHIBITORS

(71) Applicant: TAKEDA PHARMACEUTICAL COMPANY LIMITED, Osaka-shi Osaka (JP)

(72) Inventors: Nikolaos PAPAIOANNOU, Lexington, MA (US); Jeremy Mark TRAVINS, Lexington, MA (US); Sarah Jocelyn FINK, Lexington, MA (US); John Mark ELLARD, Saffron Walden (GB); Alastair RAE, Saffron Walden (GB); Jonathan Andrew SPENCER, Saffron Walden (GB)

(21) Appl. No.: 18/550,571

(22) PCT Filed: Mar. 16, 2022
(86) PCT No.: PCT/US2022/020479

§ 371 Date: Sep. 14, 2023

Related U.S. Application Data
(60) Provisional application No. 63/162,477, filed on Mar. 17, 2021.

(51) Int. Cl. A61K 31/506; C07D 519/00

(52) U.S. Cl. CPC A61K 31/506; C07D 519/00

(57) ABSTRACT

The present invention provides compounds and compositions thereof which are useful as inhibitors of plasma kallikrein and which exhibit desirable characteristics for the same.

### CROSS REFERENCE TO RELATED APPLICATIONS

This application claims the benefit of U.S. Provisional Application No. 63/162,477, filed Mar. 17, 2021, which is herein incorporated by reference in its entirety.

### I. BACKGROUND OF THE INVENTION

Plasma Kallikrein (PKa) is a serine protease zymogen in blood that is converted to its catalytically active form by coagulation factor XIIa, and contributes to the innate inflammatory response and intrinsic cascade of blood coagulation. The mechanisms that lead to the activation of this pathway in vivo include interactions with polyphosphates released from activated platelets and deficiency of C1 inhibitor (C1-INH), the primary physiological inhibitor of PKa. PKa-mediated cleavage of high-molecular weight kininogen generates the potent vasodilator and pro-inflammatory nonapeptide bradykinin (BK), which activates the bradykinin 2 receptor. Subsequent cleavage of BK by carboxypeptidases generates des-Arg9-BK, which activates the B1 receptor. Both B1 and B2 receptors are expressed by vascular, glial, and neuronal cell types, with the highest levels of retinal expression detected in the ganglion cell layer and inner and outer nuclear layers. Activation of B1 and B2 receptors causes vasodilation and increases vascular permeability.

PKa is also associated with a number of disorders, such as hereditary angioedema (HAE), an autosomal dominant disease characterized by painful, unpredictable, recurrent attacks of inflammation affecting the hands, feet, face, abdomen, urogenital tract, and the larynx. Prevalence for HAE is uncertain but is estimated to be approximately 1 case per 50,000 persons without known differences among ethnic groups. HAE is caused by deficient (Type I) or dysfunctional (Type II) levels of C1-INH, which inhibits PKa, bradykinin, and other serine proteases in the blood. Individuals with hereditary angioedema (HAE) are deficient in C1-INH and consequently undergo excessive bradykinin generation, which in turn cause painful, debilitating, and potentially fatal swelling attacks. If left untreated, HAE can result in a mortality rate as high as 40% primarily due to upper airway obstruction.

### II. SUMMARY OF THE INVENTION

The present disclosure is based on, at least in part, the development of a number of compounds which bind to plasma kallikrein and effectively inhibit its activity. Accordingly, provided herein are compounds and uses thereof for targeting plasma kallikrein and/or treating plasma kallikrein-mediated diseases and disorders.

In some embodiments, the present invention provides a compound of Formula (I):

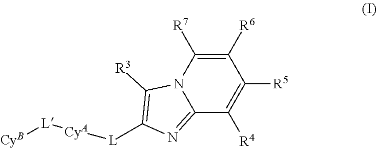

or a pharmaceutically acceptable salt thereof, wherein each of Cy^{A}, Cy^{B}, L, L′, R³, R⁴, R⁵, R⁶, R⁷, and R⁸ is defined and described in classes and subclasses herein, both singly and in combination. In certain embodiments, the present invention provides compounds of Formulae (I)-(VIII-c), as defined and described in classes and subclasses herein. In certain embodiments, the present invention provides novel intermediates and processes for preparing compounds disclosed herein. The disclosure also extends to pharmaceutical compositions comprising any one of the same, and use of compounds or compositions herein for treatment, in particular treatment of autoimmune disease, such as HAE or diabetic macular edema.

In some embodiments, the present invention also provides methods of using compounds of Formulae (I)-(VIII-c).

Advantageously, the compounds of the present disclosure have therapeutic activity and/or adequate levels of bioavailability and/or adequate half-life for use as a therapeutic.

### III. DETAILED DESCRIPTION OF CERTAIN EMBODIMENTS

### A. Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The abbreviations used herein have their conventional meaning within the chemical and biological arts. The chemical structures and formulae set forth herein are constructed according to the standard rules of chemical valency known in the chemical arts.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocyclyl,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In some embodiments, aliphatic groups contain 1-4 aliphatic carbon atoms. In some embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocyclyl” or “cycloalkyl”) refers to a monocyclic C₃-C₇ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “halogen” means F, Cl, Br, or I.

The term “aryl” refers to monocyclic and bicyclic ring systems having a total of five to 10 ring members, wherein at least one ring in the system is aromatic and wherein each ring in the system contains three to seven ring members. The term “aryl” may be used interchangeably with the term “aryl ring”. In some embodiments, an 8-10 membered bicyclic aryl group is an optionally substituted naphthyl ring. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-” refer to groups having 5 to 10 ring atoms, preferably 5, 6, or 9 ring atoms; having 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (or in the case of a bivalent fused heteroarylene ring system, at least one radical or point of attachment is on a heteroaromatic ring). Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted.

As used herein, the terms “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in this context in reference to a ring atom, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As used herein and unless otherwise specified, the suffix “-ene” is used to describe a bivalent group. Thus, any of the terms above can be modified with the suffix “-ene” to describe a bivalent version of that moiety. For example, a bivalent carbocycle is “carbocycylene”, a bivalent aryl ring is “arylene”, a bivalent benzene ring is “phenylene”, a bivalent heterocycle is “heterocyclylene”, a bivalent heteroaryl ring is “heteroarylene”, a bivalent alkyl chain is “alkylene”, a bivalent alkenyl chain is “alkenylene”, a bivalent alkynyl chain is “alkynylene”, and so forth.

As described herein, compounds of the invention may, when specified, contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure (e.g.,

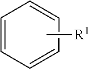

refers to at least

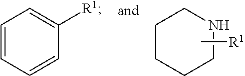

refers to at least

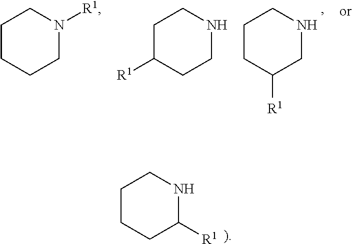

In addition, in a polycyclic ring system, substituents may, unless otherwise indicated, replace a hydrogen on any individual ring (e.g.,

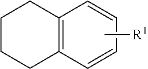

refers to at least

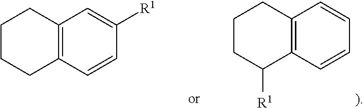

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —O(CH₂)₀₋₄C(O)OR^{o}; —O(CH₂)₀₋₄OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}, —SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄₀S(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 5-6 membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12 membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR^{#}₂, ═NNHC(O)R^{#}, ═NNHC(O)OR^{#}, ═NNHS(O)₂R^{#}, ═NR^{#}, ═NOR^{#}, —O(C(R^{#}₂))₂₋₃O—, or —S(C(R^{#}₂))₂₋₃S—, wherein each independent occurrence of R^{#} is selected from hydrogen, —CN, C₁₋₆ aliphatic which may be substituted as defined below, or an unsubstituted 5-6 membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{#}₂)₂₋₃O—, wherein each independent occurrence of R^{#} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{#} include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6 membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s) form an unsubstituted 3-12 membered saturated, partially unsaturated, or aryl mono- or bicyclic ring having 0-4 heteroatoms independently selected from nitrogen, oxygen, or sulfur.

Suitable substituents on the aliphatic group of R are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, I-19, incorporated herein by reference.

In certain embodiments, the neutral forms of the compounds are regenerated by contacting the salt with a base or acid and isolating the parent compound in the conventional manner. In some embodiments, the parent form of the compound differs from the various salt forms in certain physical properties, such as solubility in polar solvents.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

In some embodiments, compounds of the present disclosure are provided as a single enantiomer or single diastereoisomer. Single enantiomer refers to an enantiomeric excess of 80% or more, such as 81, 82, 83, 84, 85, 86, 87, 88, 89, 90, 91, 92, 93, 94, 95, 96, 97, 98 or 99%. Single diastereoisomer excess refers to an excess of 80% or more, for example 81, 82, 83, 84, 85, 86, 87, 88, 89, 90, 91, 92, 93, 94, 95, 96, 97, 98 or 99%.

The term “oxo,” as used herein, means an oxygen that is double bonded to a carbon atom, thereby forming a carbonyl.

The symbol “ [symbol] ”, except when used as a bond to depict unknown or mixed stereochemistry, denotes the point of attachment of a chemical moiety to the remainder of a molecule or chemical formula.

The articles “a” and “an” are used herein to refer to one or to more than one (i.e., to at least one) of the grammatical object of the article. By way of example, “an element” means one element or more than one element.

A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses.

As will be understood from context, a “reference” compound is one that is sufficiently similar to a particular compound of interest to permit a relevant comparison. In some embodiments, information about a reference compound is obtained simultaneously with information about a particular compound. In some embodiments, information about a reference compound is historical. In some embodiments, information about a reference compound is stored, for example in a computer-readable medium. In some embodiments, comparison of a particular compound of interest with a reference compound establishes identity with, similarity to, or difference of the particular compound of interest relative to the compound. As used herein, the phrase “therapeutic agent” refers to any agent that has a therapeutic effect and/or elicits a desired biological and/or pharmacological effect, when administered to a subject.

As used herein, the term “therapeutically effective amount” refers to an amount of a therapeutic agent that confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic agent effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific therapeutic agent employed; the specific composition employed; the age, body weight, general health, sex and diet of the subject; the time of administration, route of administration, and/or rate of excretion or metabolism of the specific therapeutic agent employed; the duration of the treatment; and like factors as is well known in the medical arts.

As used herein, the term “treatment” (also “treat” or “treating”) refers to any administration of a substance (e.g., provided compositions) that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### B. Compounds

In some embodiments, a provided compound is of Formula (I):

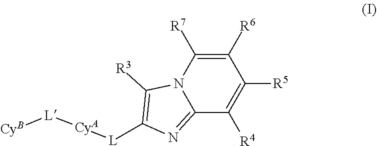

or a pharmaceutically acceptable salt thereof,
wherein:
Cy^{A} is a 4-membered monocyclic carbocyclene, a 3- to 7-membered saturated or partially unsaturated monocyclic heterocyclene having 1-3 heteroatoms selected from oxygen, nitrogen, or sulfur, phenylene, a 5- to 6-membered monocyclic heteroarylene having 1-3 heteroatoms independently selected from oxygen, nitrogen, and sulfur, a 7- to 10-membered saturated or partially unsaturated bicyclic heterocyclene having 1-4 heteroatoms selected from oxygen, nitrogen, or sulfur, or an 8- to 12-membered bicyclic heteroarylene having 1-4 heteroatoms independently selected from oxygen, nitrogen, and sulfur, wherein Cy^{A} is substituted with 0-4-R^{A} groups;
   each R^{A} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, 3- to 7-membered saturated or partially unsaturated monocyclic carbocyclyl, 3- to 7-membered saturated or partially unsaturated monocyclic heterocyclyl having 1-2 heteroatoms selected from oxygen, nitrogen, or sulfur, or a 5- to 6-membered monocyclic heteroaryl having 1-4 heteroatoms independently selected from oxygen, nitrogen or sulfur;
each R is independently hydrogen or an optionally substituted C₁₋₆ aliphatic group;
Cy^{B} is selected from phenyl, 8- to 10-membered bicyclic aryl, 7- to 10-membered saturated or partially unsaturated bicyclic carbocycyl, a 5- to 6-membered heteroaryl having 1-3 heteroatoms independently selected from oxygen, nitrogen, and sulfur, a 7- to 10-membered saturated or partially unsaturated bicyclic heterocycyl having 1-3 heteroatoms selected from oxygen, nitrogen, or sulfur, or a 7- to 10-membered heteroaryl having 1-4 heteroatoms independently selected from oxygen, nitrogen, and sulfur, wherein Cy^{B} is substituted with 0-5-R^{B} groups; or
   each R^{B} is independently selected from oxo, halogen, —CN, —C(O)R, —C(O)₂R, —C(O)N(R)₂, —C(NR)NR₂, —C(NR)NROR, —C(NR)NRC(O)OR, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, a 3- to 7-membered saturated or partially unsaturated monocyclic heterocyclyl having 1-2 heteroatoms selected from oxygen, nitrogen, or sulfur, or a 5- to 6-membered heteroaryl having 1-4 heteroatoms independently selected from oxygen, nitrogen, and sulfur;
L′ is a covalent bond or an optionally substituted C₁₋₄ hydrocarbon chain, wherein 1 to 3 methylene units are optionally and independently replaced with —O—, —C(O)—, —NR^{z}—, —S—, —SO—, SO₂—, —S(NH)(O)—, or cyclopropylene;
each R^{z} is independently selected from hydrogen, —(CH₂)₀₋₃OR, —(CH₂)₀₋₃C(O)OR, or an optionally substituted C₁₋₆ aliphatic group;
L is an optionally substituted C₁₋₂ hydrocarbon chain, wherein 1 methylene unit is optionally and independently replaced with —C(O)—, —O—, —NR^{z}—, —S—, —SO—, or —SO₂—; or L is an optionally substituted 5- to 6-membered saturated or partially unsaturated heterocyclene, having 1-3 heteroatoms independently selected from oxygen, nitrogen, and sulfur;
each R³, R⁴, R⁵, R⁶, and R⁷ is independently selected from hydrogen or -L^{C}-R^{C}, wherein
   each L^{C} is independently selected from a covalent bond or an optionally substituted C₁₋₆ hydrocarbon chain, wherein 1 to 3 methylene units are optionally and independently replaced with —O— or —NR—;
   each R^{C} is independently selected from halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, Cy^{C}, or an optionally substituted C₁₋₆ aliphatic; and
each Cy^{C} is an optionally substituted ring independently selected from a 3- to 7-membered saturated or partially unsaturated monocyclic carbocyclyl, a 3- to 7-membered saturated or partially unsaturated monocyclic heterocyclyl having 1-2 heteroatoms selected from oxygen, nitrogen, or sulfur, a 5- to 6-membered monocyclic heteroaryl having 1-3 heteroatoms independently selected from oxygen, nitrogen, and sulfur, phenyl, a 6- to 12-membered saturated or partially unsaturated fused bicyclic heterocyclyl having 1-3 heteroatoms independently selected from oxygen, nitrogen, or sulfur, a bridged bicycle, or a 6- to 12-membered saturated or partially unsaturated bicyclic spiroheterocyclyl having 1-3 heteroatoms independently selected from oxygen, nitrogen, or sulfur.

In some embodiments, a provided compound is of Formula (I), provided that:

when:
Cy^{B} is

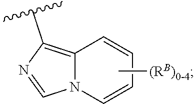

then:
L is —C(O)— or an optionally substituted C₂ hydrocarbon chain, wherein 1 methylene unit is optionally and independently replaced with —O— or —NR^{z}—; or L is an optionally substituted 5-membered saturated or partially unsaturated heterocyclene, having 1-3 heteroatoms independently selected from oxygen, nitrogen, and sulfur.

In some embodiments, a provided compound is of Formula (I), provided that:

when:
Cy^{A} is a 5-membered monocyclic heteroarylene having 1-3 heteroatoms independently selected from oxygen, nitrogen, and sulfur, wherein Cy^{A} is substituted with 0-4-R^{A} groups;
   then:
L is —C(O)— or an optionally substituted C₂ hydrocarbon chain, wherein 1 methylene unit is optionally and independently replaced with —O— or —NR^{z}—; or L is an optionally substituted 5-membered saturated or partially unsaturated heterocyclene, having 1-3 heteroatoms independently selected from oxygen, nitrogen, and sulfur.

In some embodiments, a provided compound is of Formula (I), provided that:

when:
i) Cy^{B} is

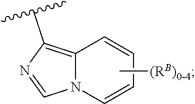

or
ii) Cy^{A} is a 5-membered monocyclic heteroarylene having 1-3 heteroatoms independently selected from oxygen, nitrogen, and sulfur, wherein Cy^{A} is substituted with 0-4-R^{A} groups;
   then:
L is —C(O)— or an optionally substituted C₂ hydrocarbon chain, wherein 1 methylene unit is optionally and independently replaced with —O— or —NR^{z}—; or L is an optionally substituted 5-membered saturated or partially unsaturated heterocyclene, having 1-3 heteroatoms independently selected from oxygen, nitrogen, and sulfur.

In some embodiments, a provided compound is of Formula (I), provided that the compound is other than:

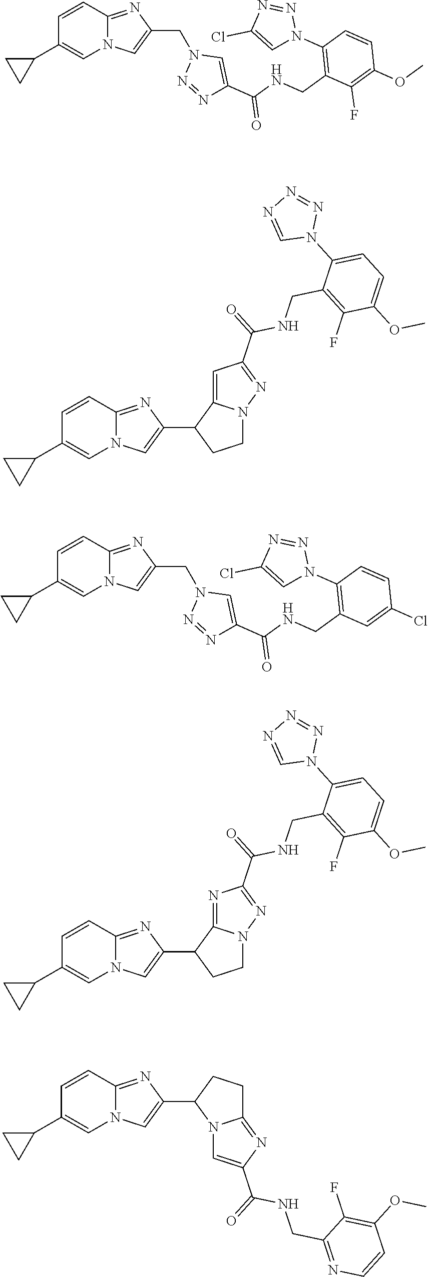 [SMILES: COc1ccc(-n2cc(Cl)nn2)c(CNC(=O)c2cn(Cc3cn4cc(C5CC5)ccc4n3)nn2)c1F ; COc1ccc(-n2cnnn2)c(CNC(=O)c2cc3n(n2)CCC3c2cn3cc(C4CC4)ccc3n2)c1F ; O=C(NCc1cc(Cl)ccc1-n1cc(Cl)nn1)c1cn(Cc2cn3cc(C4CC4)ccc3n2)nn1 ; COc1ccc(-n2cnnn2)c(CNC(=O)c2nc3n(n2)CCC3c2cn3cc(C4CC4)ccc3n2)c1F ; COc1ccnc(CNC(=O)c2cn3c(n2)CCC3c2cn3cc(C4CC4)ccc3n2)c1F]

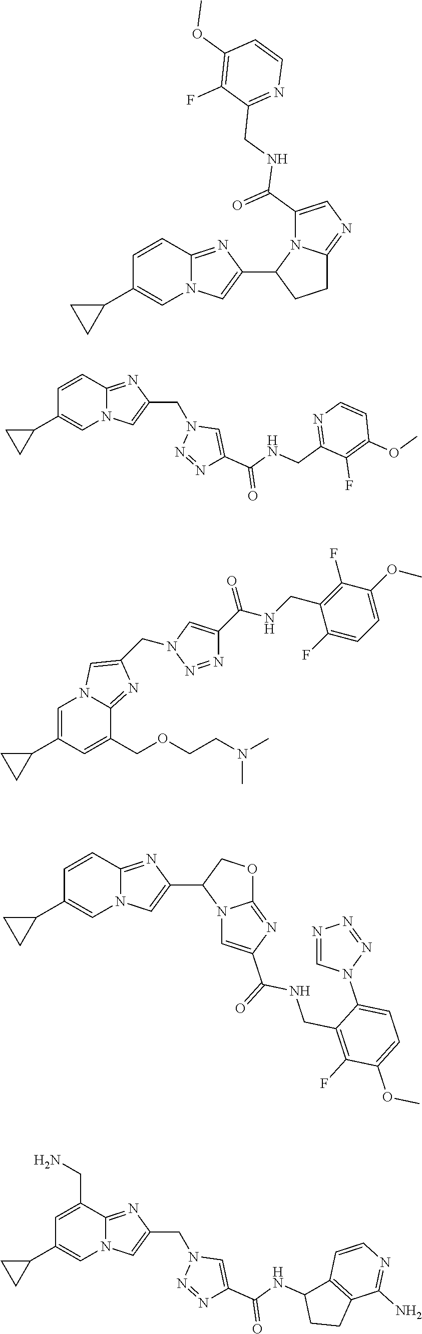

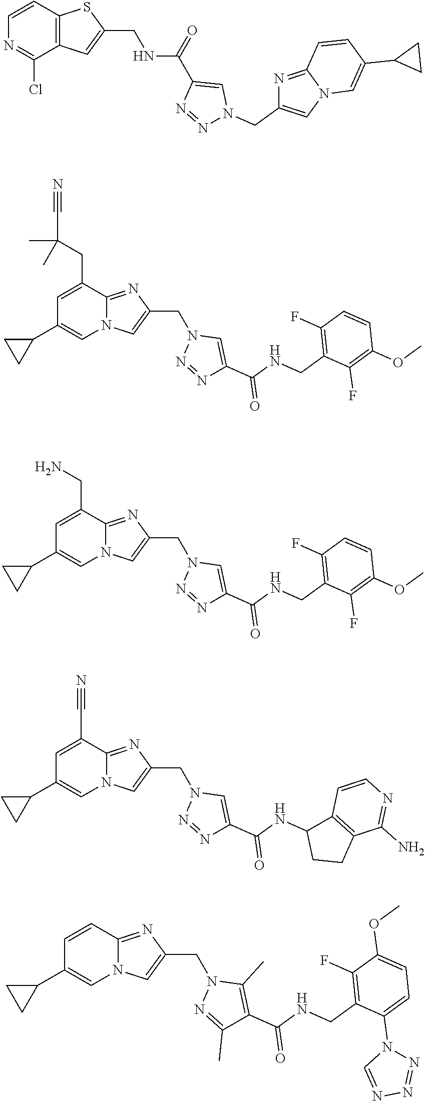 [SMILES: O=C(NCc1cc2c(Cl)nccc2s1)c1cn(Cc2cn3cc(C4CC4)ccc3n2)nn1 ; COc1ccc(F)c(CNC(=O)c2cn(Cc3cn4cc(C5CC5)cc(CC(C)(C)C#N)c4n3)nn2)c1F ; COc1ccc(F)c(CNC(=O)c2cn(Cc3cn4cc(C5CC5)cc(CN)c4n3)nn2)c1F ; N#Cc1cc(C2CC2)cn2cc(Cn3cc(C(=O)NC4CCc5c4ccnc5N)nn3)nc12 ; COc1ccc(-n2cnnn2)c(CNC(=O)c2c(C)nn(Cc3cn4cc(C5CC5)ccc4n3)c2C)c1F]

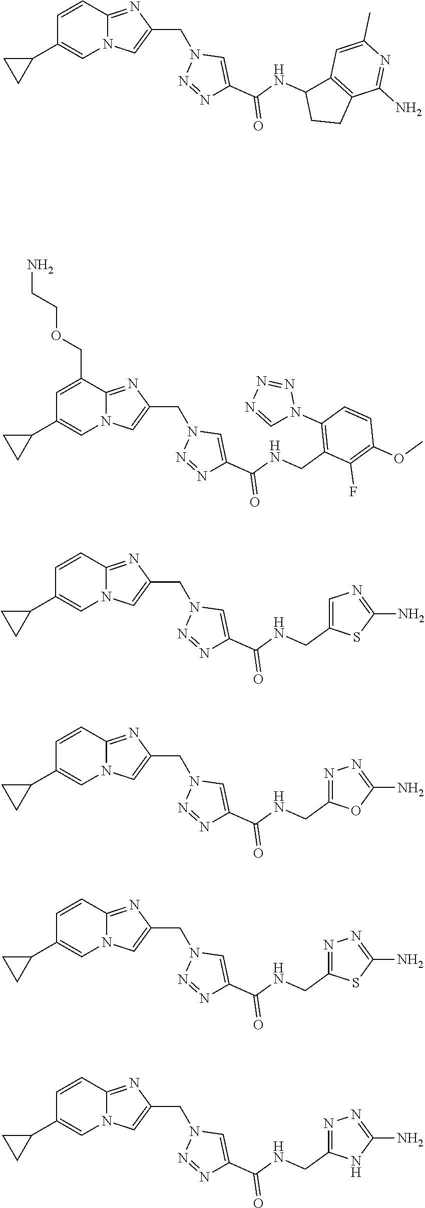 [SMILES: Cc1cc2c(c(N)n1)CCC2NC(=O)c1cn(Cc2cn3cc(C4CC4)ccc3n2)nn1 ; COc1ccc(-n2cnnn2)c(CNC(=O)c2cn(Cc3cn4cc(C5CC5)cc(COCCN)c4n3)nn2)c1F ; Nc1ncc(CNC(=O)c2cn(Cc3cn4cc(C5CC5)ccc4n3)nn2)s1 ; Nc1nnc(CNC(=O)c2cn(Cc3cn4cc(C5CC5)ccc4n3)nn2)o1 ; Nc1nnc(CNC(=O)c2cn(Cc3cn4cc(C5CC5)ccc4n3)nn2)s1 ; Nc1nnc(CNC(=O)c2cn(Cc3cn4cc(C5CC5)ccc4n3)nn2)[nH]1]

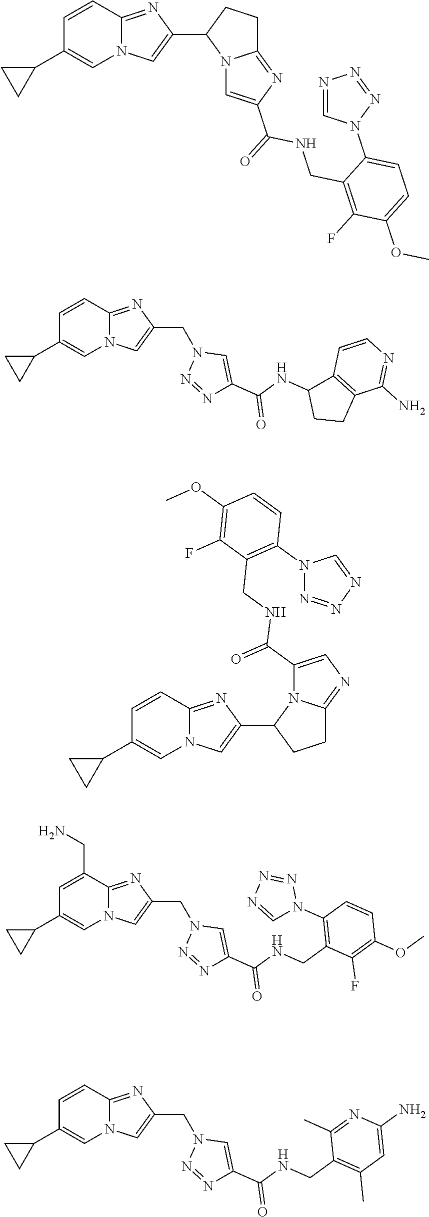 [SMILES: COc1ccc(-n2cnnn2)c(CNC(=O)c2cn3c(n2)CCC3c2cn3cc(C4CC4)ccc3n2)c1F ; Nc1nccc2c1CCC2NC(=O)c1cn(Cc2cn3cc(C4CC4)ccc3n2)nn1 ; COc1ccc(-n2cnnn2)c(CNC(=O)c2cnc3n2C(c2cn4cc(C5CC5)ccc4n2)CC3)c1F ; COc1ccc(-n2cnnn2)c(CNC(=O)c2cn(Cc3cn4cc(C5CC5)cc(CN)c4n3)nn2)c1F ; Cc1cc(N)nc(C)c1CNC(=O)c1cn(Cc2cn3cc(C4CC4)ccc3n2)nn1]

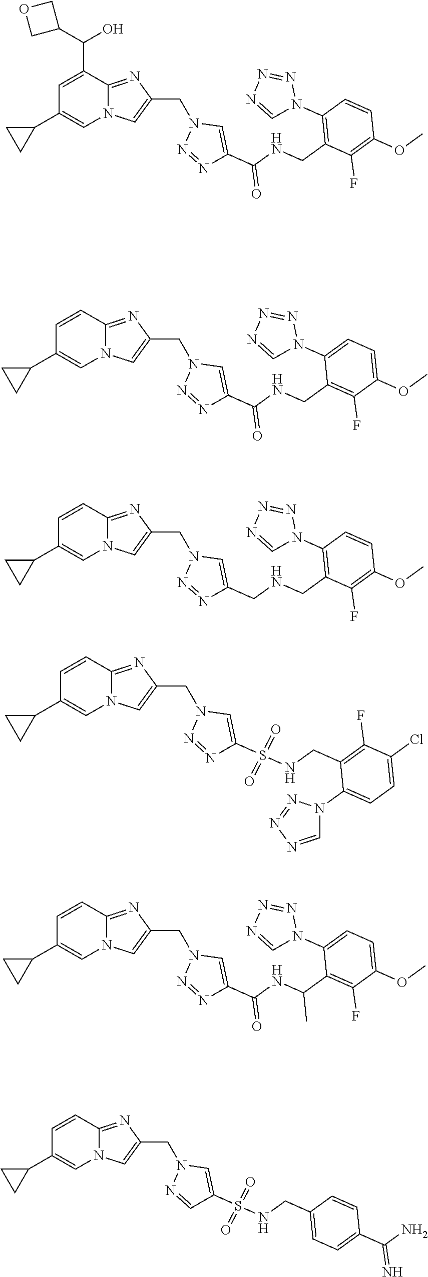 [SMILES: COc1ccc(-n2cnnn2)c(CNC(=O)c2cn(Cc3cn4cc(C5CC5)cc(C(O)C5COC5)c4n3)nn2)c1F ; COc1ccc(-n2cnnn2)c(CNC(=O)c2cn(Cc3cn4cc(C5CC5)ccc4n3)nn2)c1F ; COc1ccc(-n2cnnn2)c(CNCc2cn(Cc3cn4cc(C5CC5)ccc4n3)nn2)c1F ; O=S(=O)(NCc1c(-n2cnnn2)ccc(Cl)c1F)c1cn(Cc2cn3cc(C4CC4)ccc3n2)nn1 ; COc1ccc(-n2cnnn2)c(C(C)NC(=O)c2cn(Cc3cn4cc(C5CC5)ccc4n3)nn2)c1F ; N=C(N)c1ccc(CNS(=O)(=O)c2cnn(Cc3cn4cc(C5CC5)ccc4n3)c2)cc1]

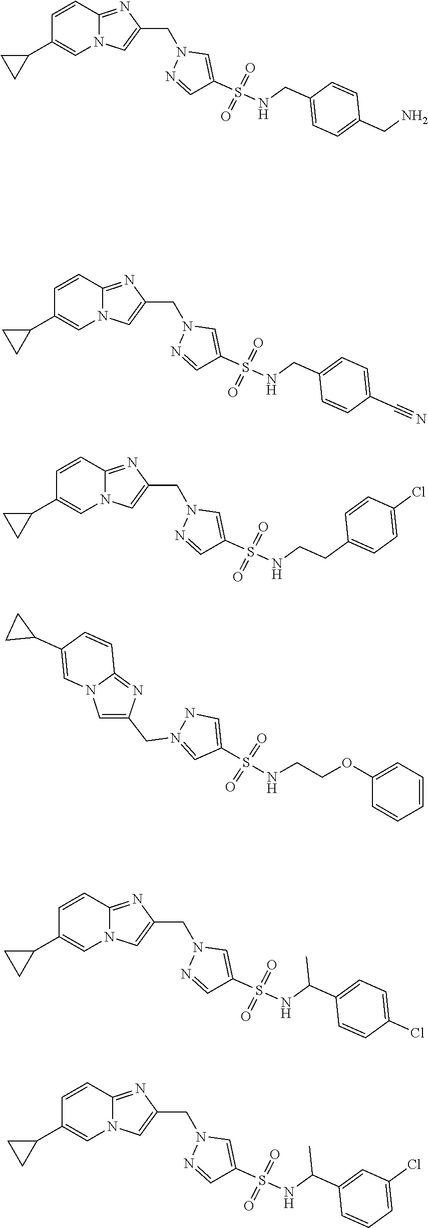

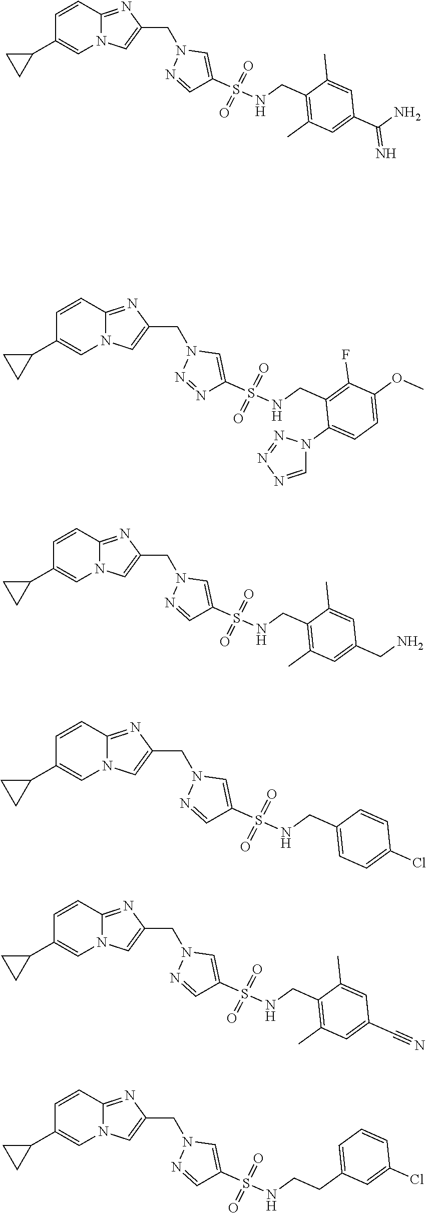 [SMILES: Cc1cc(C(=N)N)cc(C)c1CNS(=O)(=O)c1cnn(Cc2cn3cc(C4CC4)ccc3n2)c1 ; COc1ccc(-n2cnnn2)c(CNS(=O)(=O)c2cn(Cc3cn4cc(C5CC5)ccc4n3)nn2)c1F ; Cc1cc(CN)cc(C)c1CNS(=O)(=O)c1cnn(Cc2cn3cc(C4CC4)ccc3n2)c1 ; O=S(=O)(NCc1ccc(Cl)cc1)c1cnn(Cc2cn3cc(C4CC4)ccc3n2)c1 ; Cc1cc(C#N)cc(C)c1CNS(=O)(=O)c1cnn(Cc2cn3cc(C4CC4)ccc3n2)c1 ; O=S(=O)(NCCc1cccc(Cl)c1)c1cnn(Cc2cn3cc(C4CC4)ccc3n2)c1]

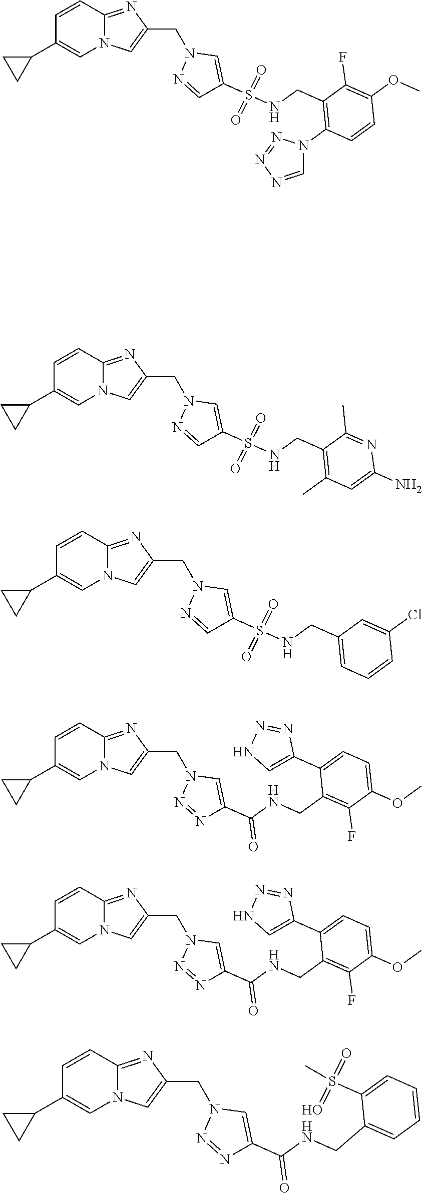 [SMILES: COc1ccc(-n2cnnn2)c(CNS(=O)(=O)c2cnn(Cc3cn4cc(C5CC5)ccc4n3)c2)c1F ; Cc1cc(N)nc(C)c1CNS(=O)(=O)c1cnn(Cc2cn3cc(C4CC4)ccc3n2)c1 ; O=S(=O)(NCc1cccc(Cl)c1)c1cnn(Cc2cn3cc(C4CC4)ccc3n2)c1 ; COc1ccc(-c2c[nH]nn2)c(CNC(=O)c2cn(Cc3cn4cc(C5CC5)ccc4n3)nn2)c1F ; COc1ccc(-c2c[nH]nn2)c(CNC(=O)c2cn(Cc3cn4cc(C5CC5)ccc4n3)nn2)c1F ; CS(=O)(=O)c1ccccc1CNC(=O)c1cn(Cc2cn3cc(C4CC4)ccc3n2)nn1]

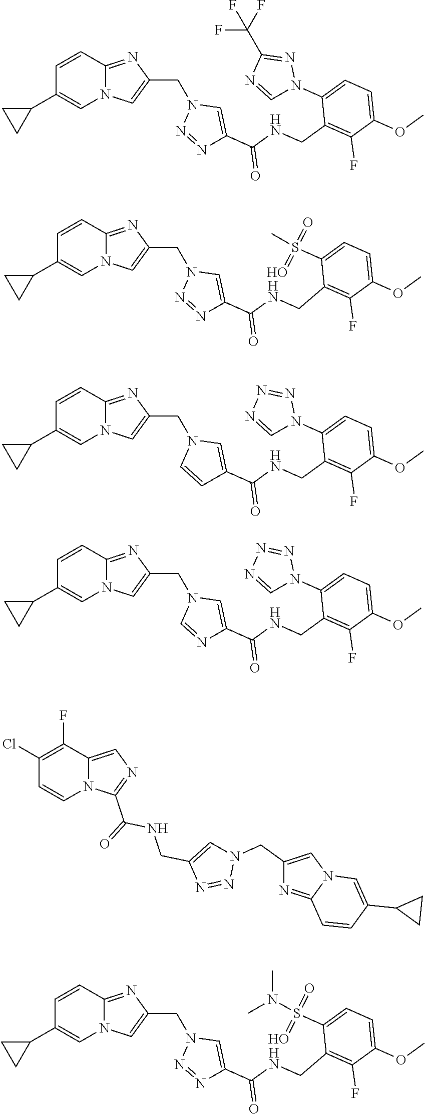

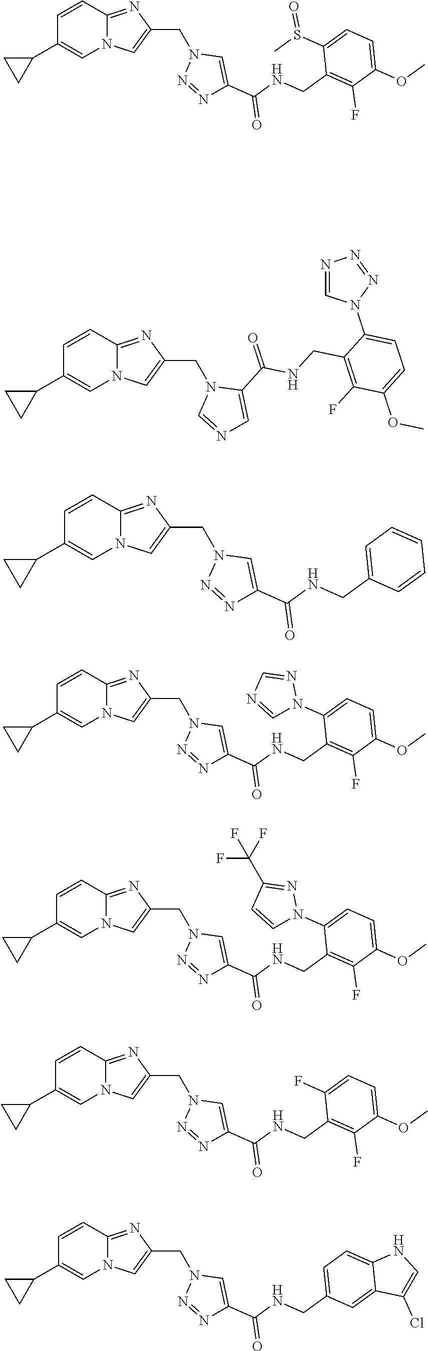

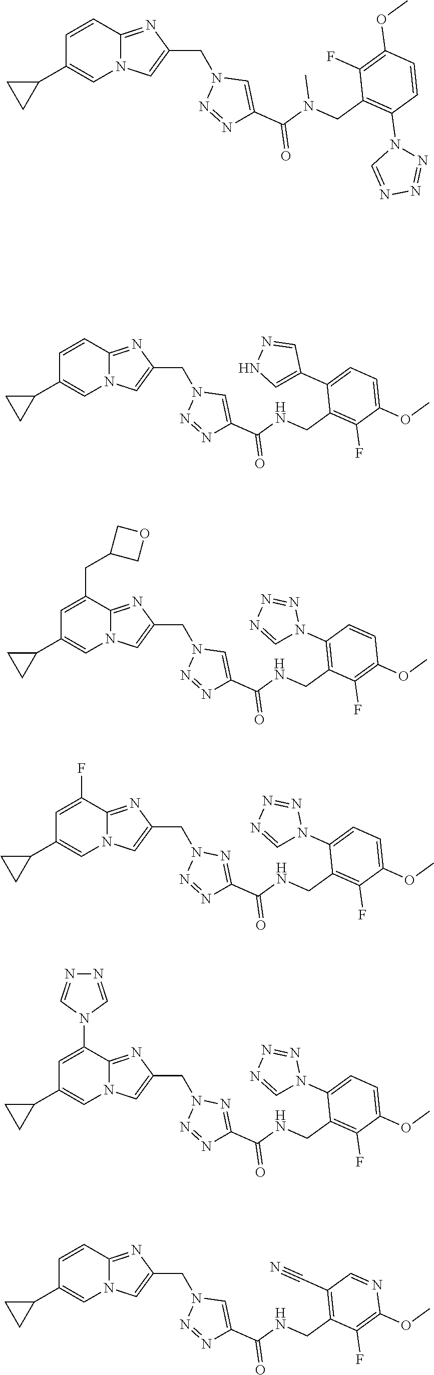 [SMILES: COc1ccc(-n2cnnn2)c(CN(C)C(=O)c2cn(Cc3cn4cc(C5CC5)ccc4n3)nn2)c1F ; COc1ccc(-c2cn[nH]c2)c(CNC(=O)c2cn(Cc3cn4cc(C5CC5)ccc4n3)nn2)c1F ; COc1ccc(-n2cnnn2)c(CNC(=O)c2cn(Cc3cn4cc(C5CC5)cc(CC5COC5)c4n3)nn2)c1F ; COc1ccc(-n2cnnn2)c(CNC(=O)c2nnn(Cc3cn4cc(C5CC5)cc(F)c4n3)n2)c1F ; COc1ccc(-n2cnnn2)c(CNC(=O)c2nnn(Cc3cn4cc(C5CC5)cc(-n5cnnc5)c4n3)n2)c1F ; COc1ncc(C#N)c(CNC(=O)c2cn(Cc3cn4cc(C5CC5)ccc4n3)nn2)c1F]

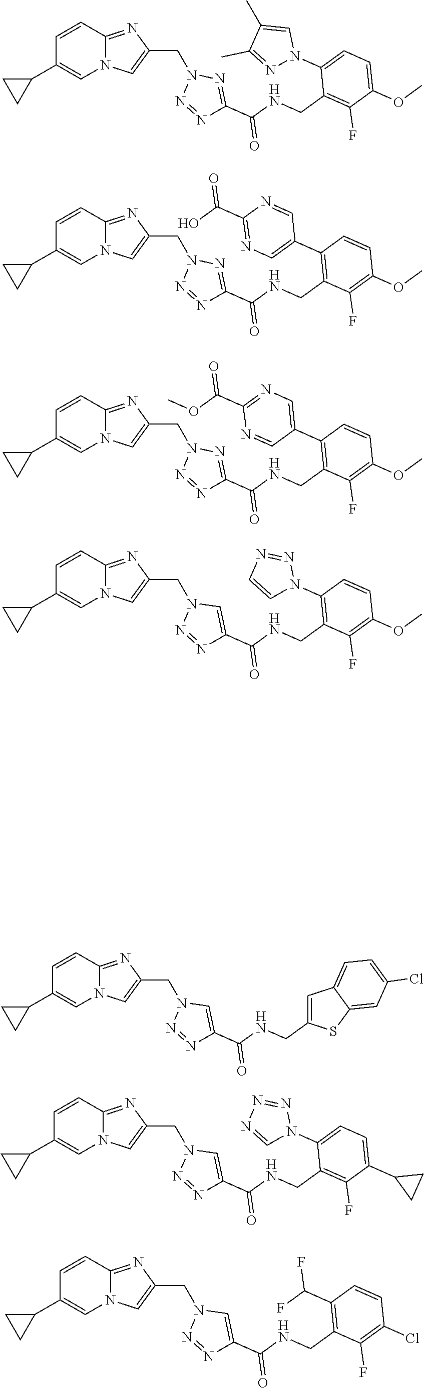

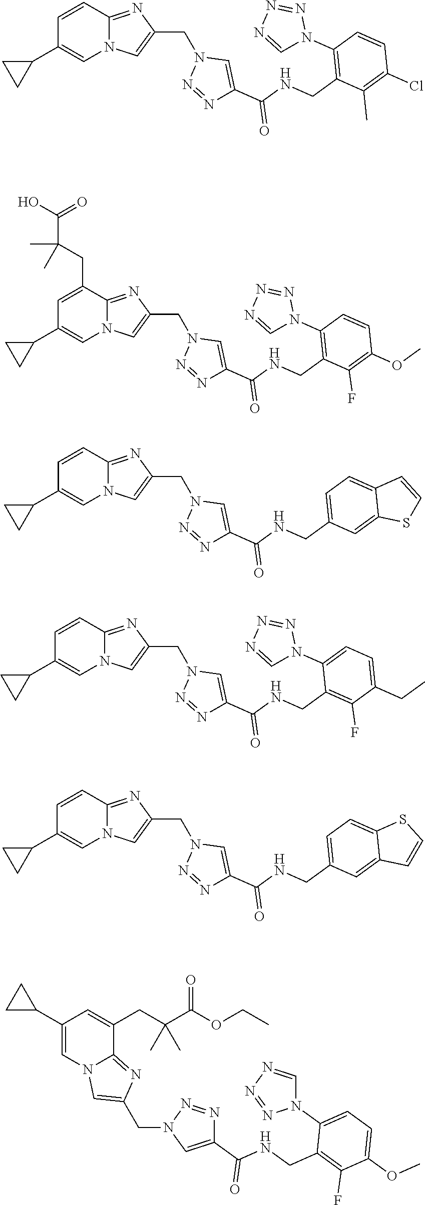 [SMILES: Cc1c(Cl)ccc(-n2cnnn2)c1CNC(=O)c1cn(Cc2cn3cc(C4CC4)ccc3n2)nn1 ; COc1ccc(-n2cnnn2)c(CNC(=O)c2cn(Cc3cn4cc(C5CC5)cc(CC(C)(C)C(=O)O)c4n3)nn2)c1F ; O=C(NCc1ccc2ccsc2c1)c1cn(Cc2cn3cc(C4CC4)ccc3n2)nn1 ; CCc1ccc(-n2cnnn2)c(CNC(=O)c2cn(Cc3cn4cc(C5CC5)ccc4n3)nn2)c1F ; O=C(NCc1ccc2sccc2c1)c1cn(Cc2cn3cc(C4CC4)ccc3n2)nn1 ; CCOC(=O)C(C)(C)Cc1cc(C2CC2)cn2cc(Cn3cc(C(=O)NCc4c(-n5cnnn5)ccc(OC)c4F)nn3)nc12]

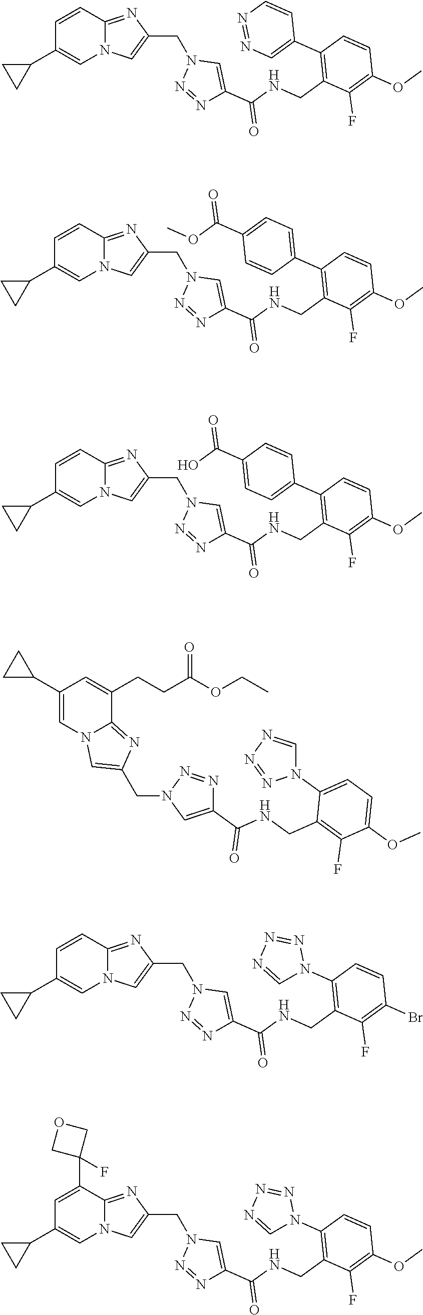 [SMILES: COc1ccc(-c2ccnnc2)c(CNC(=O)c2cn(Cc3cn4cc(C5CC5)ccc4n3)nn2)c1F ; COC(=O)c1ccc(-c2ccc(OC)c(F)c2CNC(=O)c2cn(Cc3cn4cc(C5CC5)ccc4n3)nn2)cc1 ; COc1ccc(-c2ccc(C(=O)O)cc2)c(CNC(=O)c2cn(Cc3cn4cc(C5CC5)ccc4n3)nn2)c1F ; CCOC(=O)CCc1cc(C2CC2)cn2cc(Cn3cc(C(=O)NCc4c(-n5cnnn5)ccc(OC)c4F)nn3)nc12 ; O=C(NCc1c(-n2cnnn2)ccc(Br)c1F)c1cn(Cc2cn3cc(C4CC4)ccc3n2)nn1 ; COc1ccc(-n2cnnn2)c(CNC(=O)c2cn(Cc3cn4cc(C5CC5)cc(C5(F)COC5)c4n3)nn2)c1F]

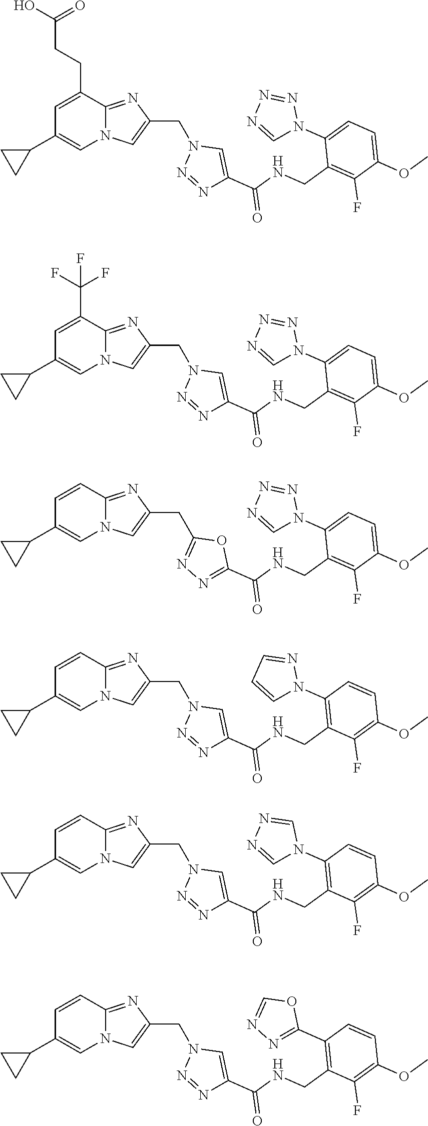

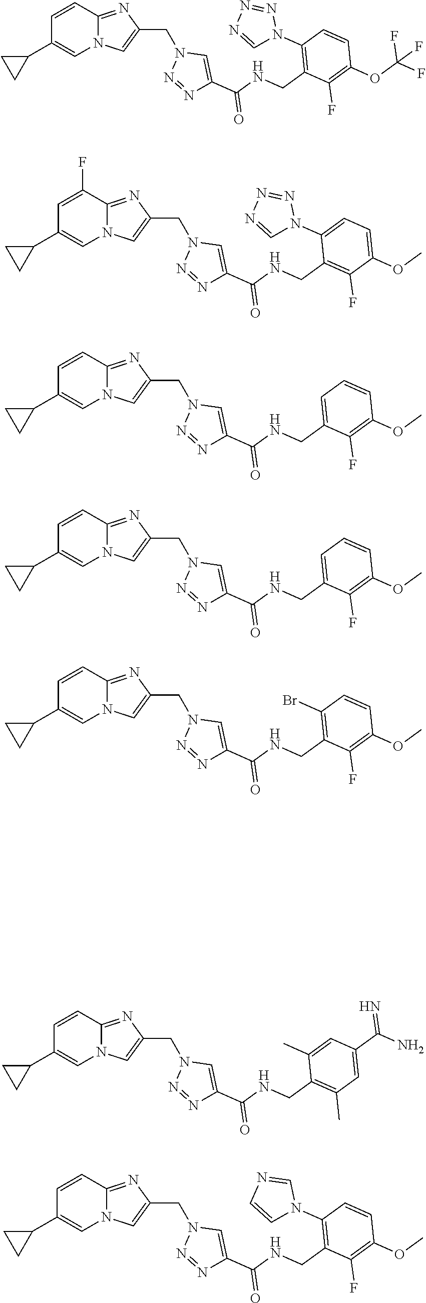

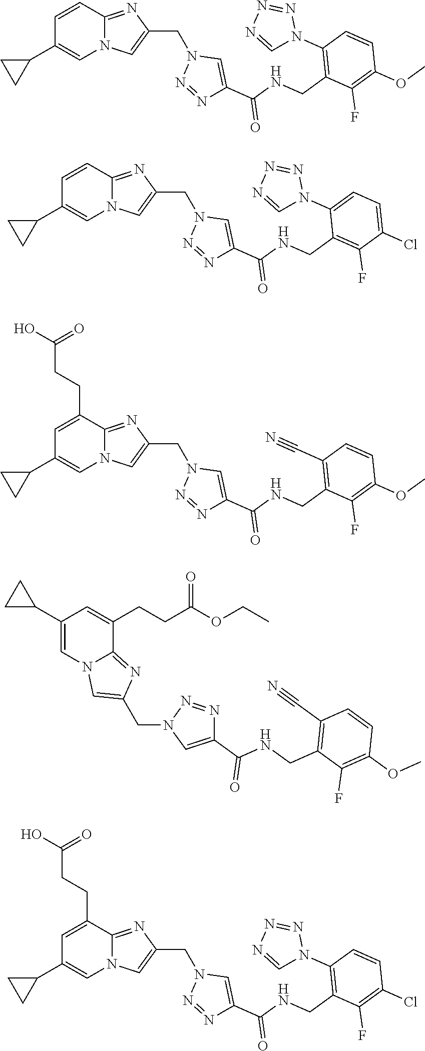 [SMILES: COc1ccc(-n2cnnn2)c(CNC(=O)c2cn(Cc3cn4cc(C5CC5)ccc4n3)nn2)c1F ; O=C(NCc1c(-n2cnnn2)ccc(Cl)c1F)c1cn(Cc2cn3cc(C4CC4)ccc3n2)nn1 ; COc1ccc(C#N)c(CNC(=O)c2cn(Cc3cn4cc(C5CC5)cc(CCC(=O)O)c4n3)nn2)c1F ; CCOC(=O)CCc1cc(C2CC2)cn2cc(Cn3cc(C(=O)NCc4c(C#N)ccc(OC)c4F)nn3)nc12 ; O=C(O)CCc1cc(C2CC2)cn2cc(Cn3cc(C(=O)NCc4c(-n5cnnn5)ccc(Cl)c4F)nn3)nc12]

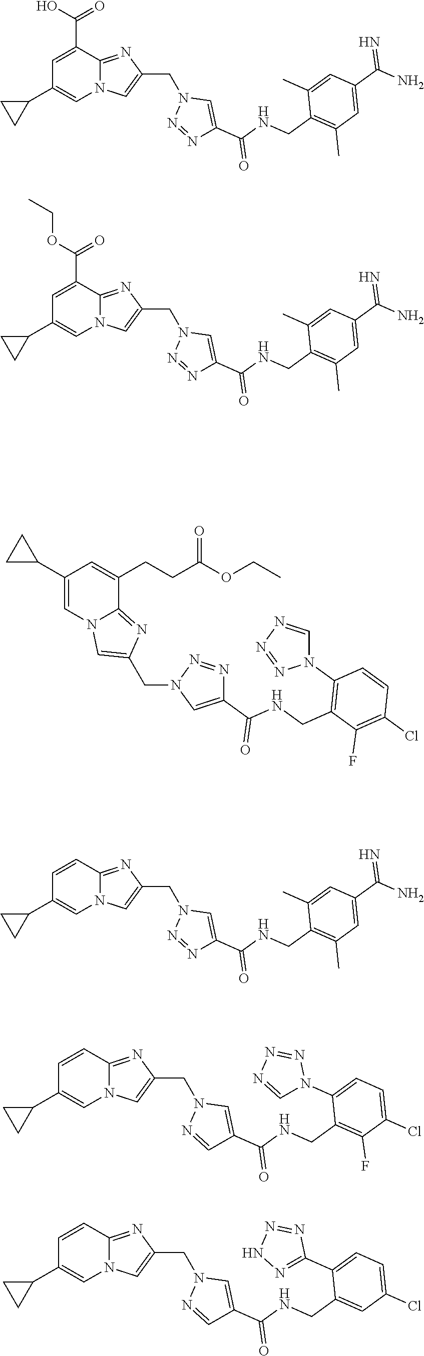 [SMILES: Cc1cc(C(=N)N)cc(C)c1CNC(=O)c1cn(Cc2cn3cc(C4CC4)cc(C(=O)O)c3n2)nn1 ; CCOC(=O)c1cc(C2CC2)cn2cc(Cn3cc(C(=O)NCc4c(C)cc(C(=N)N)cc4C)nn3)nc12 ; CCOC(=O)CCc1cc(C2CC2)cn2cc(Cn3cc(C(=O)NCc4c(-n5cnnn5)ccc(Cl)c4F)nn3)nc12 ; Cc1cc(C(=N)N)cc(C)c1CNC(=O)c1cn(Cc2cn3cc(C4CC4)ccc3n2)nn1 ; O=C(NCc1c(-n2cnnn2)ccc(Cl)c1F)c1cnn(Cc2cn3cc(C4CC4)ccc3n2)c1 ; O=C(NCc1cc(Cl)ccc1-c1nn[nH]n1)c1cnn(Cc2cn3cc(C4CC4)ccc3n2)c1]

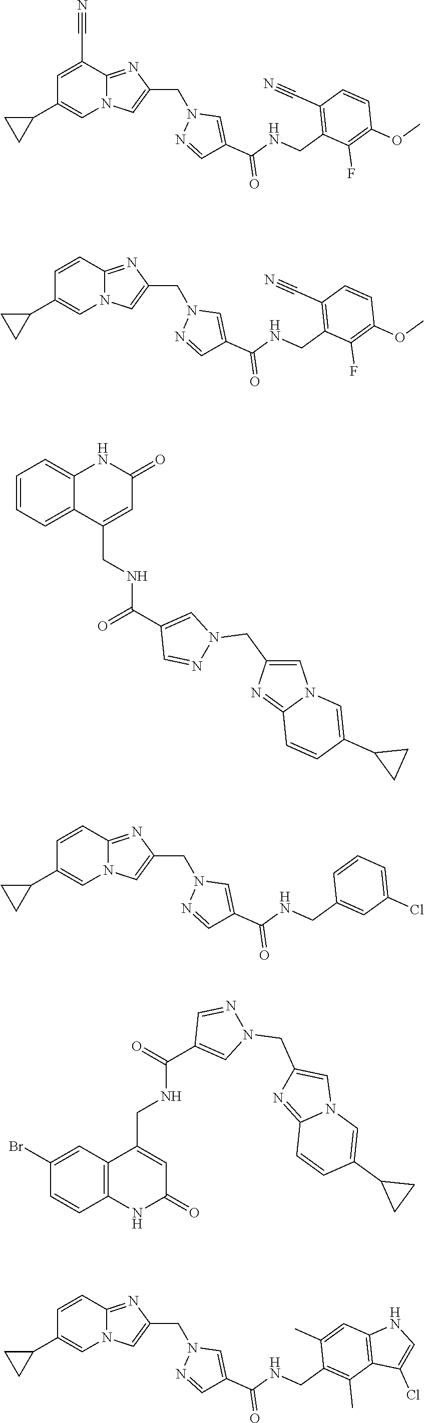 [SMILES: COc1ccc(C#N)c(CNC(=O)c2cnn(Cc3cn4cc(C5CC5)cc(C#N)c4n3)c2)c1F ; COc1ccc(C#N)c(CNC(=O)c2cnn(Cc3cn4cc(C5CC5)ccc4n3)c2)c1F ; O=C(NCc1cc(=O)[nH]c2ccccc12)c1cnn(Cc2cn3cc(C4CC4)ccc3n2)c1 ; O=C(NCc1cccc(Cl)c1)c1cnn(Cc2cn3cc(C4CC4)ccc3n2)c1 ; O=C(NCc1cc(=O)[nH]c2ccc(Br)cc12)c1cnn(Cc2cn3cc(C4CC4)ccc3n2)c1 ; Cc1cc2[nH]cc(Cl)c2c(C)c1CNC(=O)c1cnn(Cc2cn3cc(C4CC4)ccc3n2)c1]

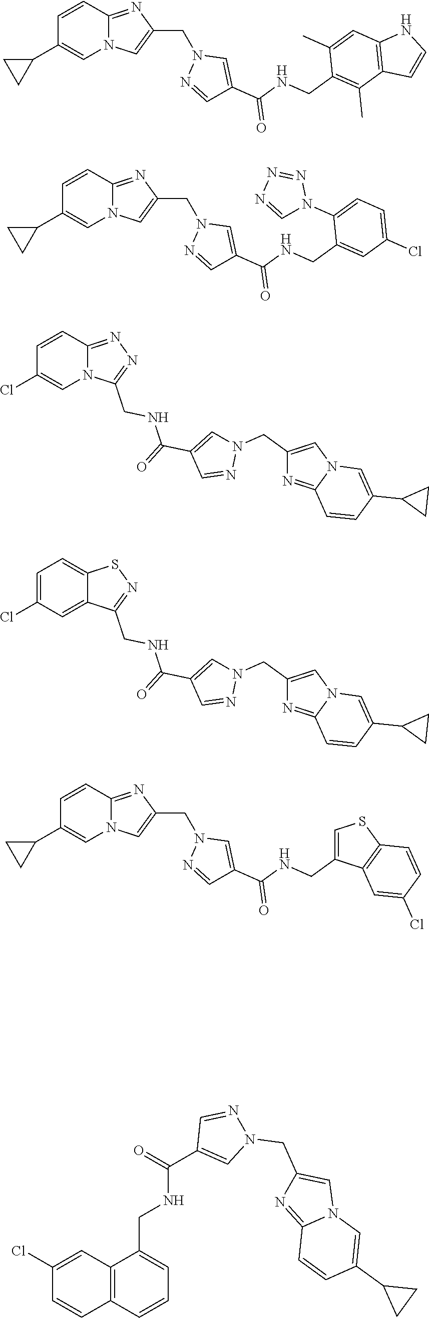 [SMILES: Cc1cc2[nH]ccc2c(C)c1CNC(=O)c1cnn(Cc2cn3cc(C4CC4)ccc3n2)c1 ; O=C(NCc1cc(Cl)ccc1-n1cnnn1)c1cnn(Cc2cn3cc(C4CC4)ccc3n2)c1 ; O=C(NCc1nnc2ccc(Cl)cn12)c1cnn(Cc2cn3cc(C4CC4)ccc3n2)c1 ; O=C(NCc1nsc2ccc(Cl)cc12)c1cnn(Cc2cn3cc(C4CC4)ccc3n2)c1 ; O=C(NCc1csc2ccc(Cl)cc12)c1cnn(Cc2cn3cc(C4CC4)ccc3n2)c1 ; O=C(NCc1cccc2ccc(Cl)cc12)c1cnn(Cc2cn3cc(C4CC4)ccc3n2)c1]

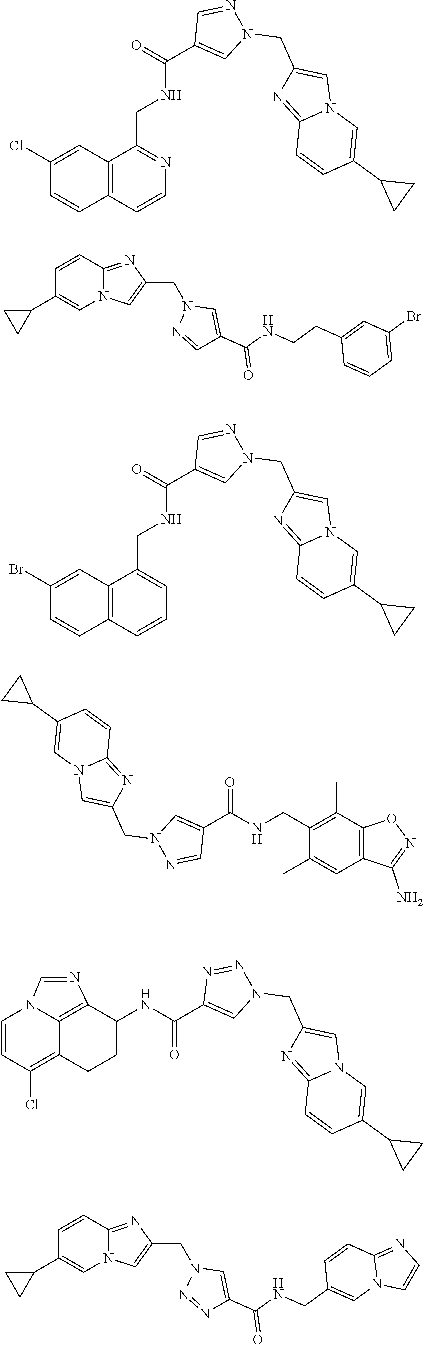

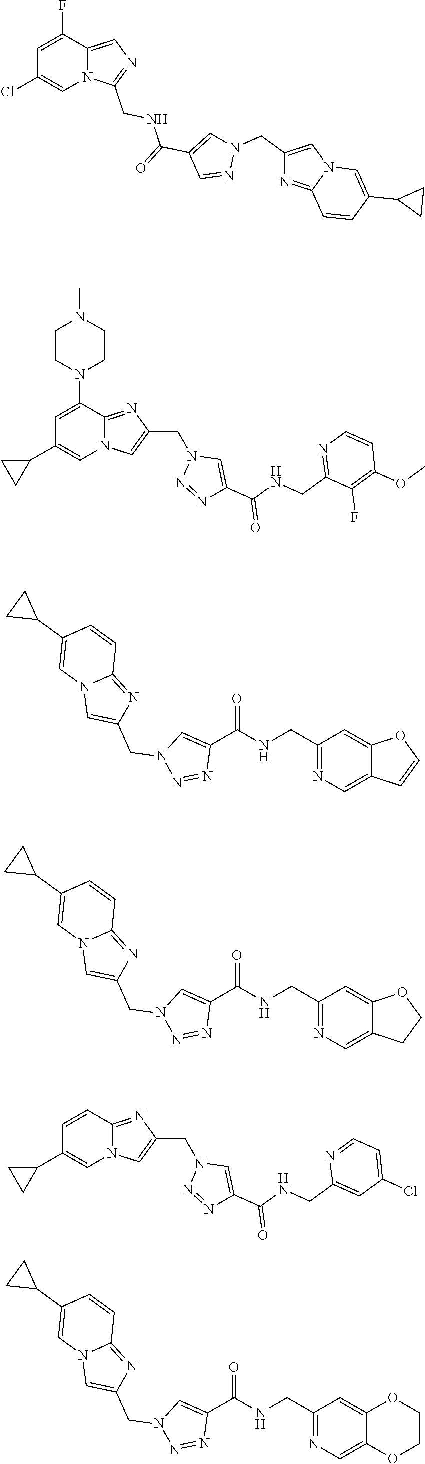 [SMILES: O=C(NCc1ncc2c(F)cc(Cl)cn12)c1cnn(Cc2cn3cc(C4CC4)ccc3n2)c1 ; COc1ccnc(CNC(=O)c2cn(Cc3cn4cc(C5CC5)cc(N5CCN(C)CC5)c4n3)nn2)c1F ; O=C(NCc1cc2occc2cn1)c1cn(Cc2cn3cc(C4CC4)ccc3n2)nn1 ; O=C(NCc1cc2c(cn1)CCO2)c1cn(Cc2cn3cc(C4CC4)ccc3n2)nn1 ; O=C(NCc1cc(Cl)ccn1)c1cn(Cc2cn3cc(C4CC4)ccc3n2)nn1 ; O=C(NCc1cc2c(cn1)OCCO2)c1cn(Cc2cn3cc(C4CC4)ccc3n2)nn1]

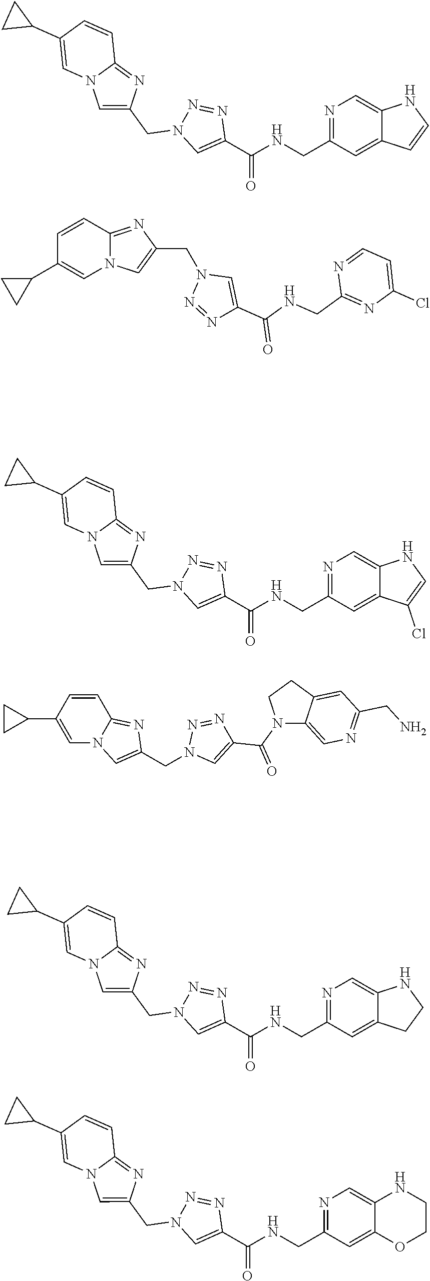 [SMILES: O=C(NCc1cc2cc[nH]c2cn1)c1cn(Cc2cn3cc(C4CC4)ccc3n2)nn1 ; O=C(NCc1nccc(Cl)n1)c1cn(Cc2cn3cc(C4CC4)ccc3n2)nn1 ; O=C(NCc1cc2c(Cl)c[nH]c2cn1)c1cn(Cc2cn3cc(C4CC4)ccc3n2)nn1 ; NCc1cc2c(cn1)N(C(=O)c1cn(Cc3cn4cc(C5CC5)ccc4n3)nn1)CC2 ; O=C(NCc1cc2c(cn1)NCC2)c1cn(Cc2cn3cc(C4CC4)ccc3n2)nn1 ; O=C(NCc1cc2c(cn1)NCCO2)c1cn(Cc2cn3cc(C4CC4)ccc3n2)nn1]

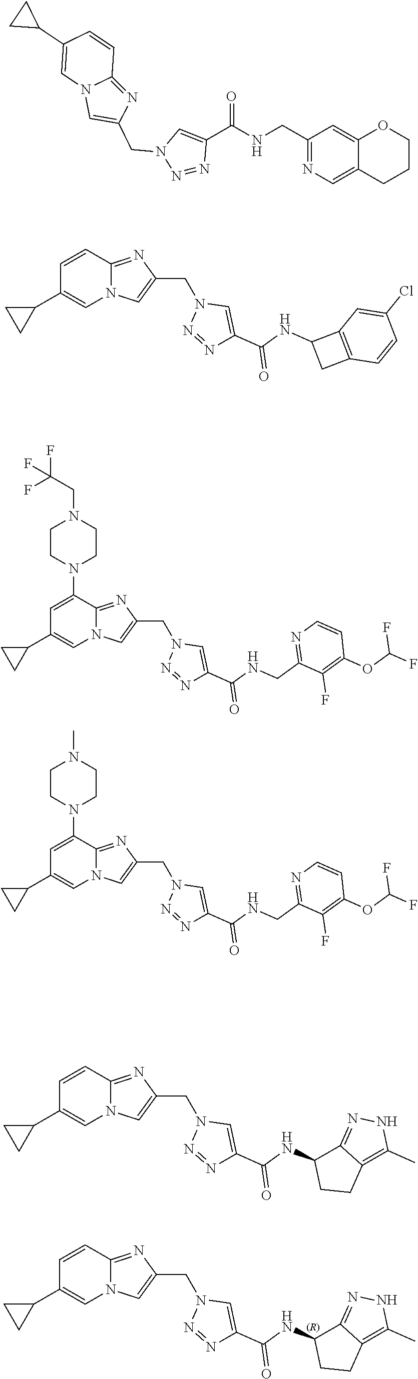

2-(7-chloroimidazo[1,5-a]pyridin-1-yl)-N-((1-((6-cyclopropylimidazo[1,2-a]pyridin-2-yl)methyl)-1H-1,2,3-triazol-4-yl)methyl)acetamide, 1-(7-chloro-8-fluoroimidazo[1,5-a]pyridin-1-yl)-N-((1-((6-cyclopropylimidazo[1,2-a]pyridin-2-yl)methyl)-1H-1,2,3-triazol-4-yl)methyl)-2,2,2-trifluoroethan-1-amine, 7-chloro-N-((1-((6-cyclopropylimidazo[1,2-a]pyridin-2-yl)methyl)-1H-1,2,3-triazol-4-yl)methyl)-8-fluoroimidazo[1,5-a]pyridine-1-carboxamide, N-((7-chloro-8-fluoroimidazo[1,5-a]pyridin-1-yl)methyl)-1-((6-cyclopropylimidazo[1,2-a]pyridin-2-yl)methyl)-1H-pyrazole-4-sulfonamide, N-((7-chloro-8-fluoroimidazo[1,5-a]pyridin-1-yl)methyl)-1-((6-cyclopropylimidazo[1,2-a]pyridin-2-yl)methyl)-1H-1,2,3-triazole-4-sulfonamide, 7-chloro-N-((1-((6-cyclopropylimidazo[1,2-a]pyridin-2-yl)methyl)-1H-1,2,3-triazol-4-yl)methyl)-8-fluoroimidazo[1,5-a]pyridine-1-sulfonamide, 7-chloro-N-(1-(1-((6-cyclopropylimidazo[1,2-a]pyridin-2-yl)methyl)-1H-1,2,3-triazol-4-yl)ethyl)-8-fluoroimidazo[1,5-a]pyridine-1-sulfonamide, N-((7-chloro-8-fluoroimidazo[1,5-a]pyridin-1-yl)methyl)-5-(6-cyclopropylimidazo[1,2-a]pyridin-2-yl)-6,7-dihydro-5H-pyrrolo[1,2-a]imidazole-2-carboxamide, N-((7-chloro-8-fluoroimidazo[1,5-a]pyridin-1-yl)methyl)-5-(6-cyclopropylimidazo[1,2-a]pyridin-2-yl)-6,7-dihydro-5H-pyrrolo[1,2-a]imidazole-3-carboxamide, N-((7-cyanoimidazo[1,5-a]pyridin-1-yl)methyl)-1-((6-cyclopropylimidazo[1,2-a]pyridin-2-yl)methyl)-1H-pyrazole-4-carboxamide, 1-(7-chloro-8-fluoroimidazo[1,5-a]pyridin-1-yl)-N-((1-((6-cyclopropylimidazo[1,2-a]pyridin-2-yl)methyl)-1H-1,2,3-triazol-4-yl)methyl)methanamine, N-((7-chloro-8-fluoroimidazo[1,5-a]pyridin-1-yl)methyl)-1-(1-((6-cyclopropylimidazo[1,2-a]pyridin-2-yl)methyl)-1H-1,2,3-triazol-4-yl)ethan-1-amine, 2-(7-chloroimidazo[1,5-a]pyridin-1-yl)-N-(1-(1-((6-cyclopropylimidazo[1,2-a]pyridin-2-yl)methyl)-1H-1,2,3-triazol-4-yl)ethyl)acetamide, methyl 2-((4-((((7-chloro-8-fluoroimidazo[1,5-a]pyridin-1-yl)methyl)amino)methyl)-1H-1,2,3-triazol-1-yl)methyl)-6-cyclopropylimidazo[1,2-a]pyridine-8-carboxylate, 2-((4-((((7-chloro-8-fluoroimidazo[1,5-a]pyridin-1-yl)methyl)amino)methyl)-1H-1,2,3-triazol-1-yl)methyl)-6-cyclopropylimidazo[1,2-a]pyridine-8-carboxylic acid, N-((7-chloro-8-fluoroimidazo[1,5-a]pyridin-1-yl)methyl)-1-(1-((6-cyclopropylimidazo[1,2-a]pyridin-2-yl)methyl)-1H-pyrazol-4-yl)-2,2,2-trifluoroethan-1-amine, ethyl 3-(2-((4-((((7-chloro-8-fluoroimidazo[1,5-a]pyridin-1-yl)methyl)amino)methyl)-1H-1,2,3-triazol-1-yl)methyl)-6-cyclopropylimidazo[1,2-a]pyridin-8-yl)propanoate, 3-(2-((4-((((7-chloro-8-fluoroimidazo[1,5-a]pyridin-1-yl)methyl)amino)methyl)-1H-1,2,3-triazol-1-yl)methyl)-6-cyclopropylimidazo[1,2-a]pyridin-8-yl)propanoic acid, 1-(7-chloro-8-fluoroimidazo[1,5-a]pyridin-1-yl)-N-((1-((6-cyclopropyl-8-(4H-1,2,4-triazol-4-yl)imidazo[1,2-a]pyridin-2-yl)methyl)-1H-1,2,3-triazol-4-yl)methyl)methanamine, 2-(7-chloroimidazo[1,5-a]pyridin-1-yl)-N-(1-(1-((6-cyclopropylimidazo[1,2-a]pyridin-2-yl)methyl)-1H-pyrazol-4-yl)-2,2,2-trifluoroethyl)acetamide, N-((7-chloro-8-fluoroimidazo[1,5-a]pyridin-1-yl)methyl)-1-((6-cyclopropyl-8-(4-methylpiperazin-1-yl)imidazo[1,2-a]pyridin-2-yl)methyl)-1H-1,2,3-triazole-4-carboxamide, or a pharmaceutically acceptable salt thereof.

It will be appreciated that, “oxo” refers a double bonded oxygen substitution on a carbon “C═O”, where the carbon atom is part of the structure or group that is substituted by oxo. For example, where Cy^{C} is substituted with -L^{D}-R^{D}, and where L^{D} is a covalent bond and R^{D} is oxo, the carbon atom substituted with oxo (i.e., the carbon in C═O) is part of Cy^{C} (e.g., a structure of Cy^{C} being cyclopentyl substituted with -L^{D}-R^{D} at the 2-position, where L^{D} is a covalent bond and R^{D} is oxo corresponds to

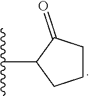 [SMILES: *C1CCCC1=O]

In some embodiments, Cy^{A} is a phenylene or a 5- to 6-membered monocyclic heteroarylene having 1-3 heteroatoms independently selected from oxygen, nitrogen, and sulfur, wherein Cy^{A} is substituted with 0-4 R^{A} groups. In some embodiments, Cy^{A} is a 5- to 6-membered monocyclic heteroarylene having 1-3 heteroatoms independently selected from oxygen, nitrogen, and sulfur, or an 8- to 12-membered bicyclic heteroarylene having 1-4 heteroatoms independently selected from oxygen, nitrogen, and sulfur, wherein Cy^{A} is substituted with 0-4-R^{A} groups.

In certain embodiments, Cy^{A} is a 4-membered monocyclic carbocyclene, a 3- to -7 membered saturated or partially unsaturated monocyclic heterocyclene having 1-3 heteroatoms selected from oxygen, nitrogen, or sulfur, a 5- to 6-membered monocyclic heteroarylene having 1-3 heteroatoms independently selected from oxygen, nitrogen, and sulfur, a 7- to 10-membered saturated or partially unsaturated bicyclic heterocyclene having 1-4 heteroatoms selected from oxygen, nitrogen, or sulfur, or a 7- to 10-membered bicyclic heteroarylene having 1-4 heteroatoms independently selected from oxygen, nitrogen, and sulfur, wherein Cy^{A} is substituted with 0-4-R^{A} groups.

In some embodiments, Cy^{A} is a 4-membered monocyclic carbocyclene, wherein Cy^{A} is substituted with 0-4-R^{A} groups. In some embodiments, Cy^{A} is cyclobutendionediyl, wherein Cy^{A} is substituted with 0-2-R^{A} groups. In some embodiments, Cy^{A} is

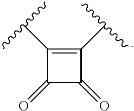 [SMILES: *c1c(*)c(=O)c1=O]

In some embodiments, Cy^{A} is a phenylene, wherein Cy^{A} is substituted with 0-4-R^{A} groups. In some embodiments, Cy^{A} is a phenylene, wherein Cy^{A} is substituted with 0-2-R^{A} groups.

In some embodiments, Cy^{A} is a 5- to 6-membered monocyclic heteroarylene having 1-3 heteroatoms independently selected from oxygen, nitrogen, and sulfur, wherein Cy^{A} is substituted with 0-4-R^{A} groups.

In some embodiments, Cy^{A} is a 6-membered monocyclic heteroarylene having 1-3 heteroatoms independently selected from oxygen, nitrogen, and sulfur, wherein Cy^{A} is substituted with 0-4-R^{A} groups. In some embodiments, Cy^{A} is a 6-membered monocyclic heteroarylene having 1-3 nitrogen heteroatoms, wherein Cy^{A} is substituted with 0-4 R^{A} groups. In some embodiments, Cy^{A} is a pyridinediyl substituted with 0-3 R^{A} groups. In some embodiments, Cy^{A} is a pyrimidinediyl substituted with 0-2 R^{A} groups. In some embodiments, Cy^{A} is a pyridazinediyl substituted with 0-2 R^{A} groups. In some embodiments, Cy^{A} is a pyridinediyl substituted with 0-1 R^{A} groups. In some embodiments, Cy^{A} is a pyrimidinediyl substituted with 0-1 R^{A} groups. In some embodiments, Cy^{A} is a pyridazinediyl substituted with 0-1 R^{A} groups. In some embodiments, Cy^{A} is a triazinediyl substituted with 0-1 R^{A} groups.

In some embodiments, Cy^{A} is a 5-membered monocyclic heteroarylene having 1-3 heteroatoms independently selected from oxygen, nitrogen, and sulfur, wherein Cy^{A} is substituted with 0-2-R^{A} groups. In some embodiments, Cy^{A} is an unsubstituted thiadiazolediyl. In some embodiments, Cy^{A} is an unsubstituted oxadiazolediyl. In some embodiments, Cy^{A} is an unsubstituted triazolediyl. In some embodiments, Cy^{A} is a thiazolediyl, substituted with 0-3 R^{A} groups.

In some embodiments, Cy^{A} is 3- to 7-membered saturated or partially unsaturated monocyclic heterocyclene having 1-3 heteroatoms selected from oxygen, nitrogen, or sulfur, wherein Cy^{A} is substituted with 0-4-R^{A} groups.

In some embodiments, Cy^{A} is a 7- to 10-membered saturated or partially unsaturated bicyclic heterocyclene having 1-4 heteroatoms selected from oxygen, nitrogen, or sulfur, wherein Cy^{A} is substituted with 0-4-R^{A} groups. In some embodiments, Cy^{A} is a 9-membered saturated or partially unsaturated bicyclic heterocyclene having 1-4 heteroatoms selected from oxygen, nitrogen, or sulfur, wherein Cy^{A} is substituted with 0-4-R^{A} groups. In some embodiments, Cy^{A} is a 9-membered saturated or partially unsaturated bicyclic heterocyclene having 2 nitrogen heteroatoms, wherein Cy^{A} is substituted with 0-4-R^{A} groups. In some embodiments, Cy^{A} is a 10-membered saturated or partially unsaturated bicyclic heterocyclene having 1-4 heteroatoms selected from oxygen, nitrogen, or sulfur, wherein Cy^{A} is substituted with 0-4-R^{A} groups. In some embodiments, Cy^{A} is a 10-membered saturated or partially unsaturated bicyclic heterocyclene having 2 nitrogen heteroatoms, wherein Cy^{A} is substituted with 0-4-R^{A} groups. In some embodiments, Cy^{A} is dihydroindazolonediyl substituted with 0-4 R^{A} groups. In some embodiments, Cy^{A} is quinazolinonediyl substituted with 0-4 R^{A} groups.

In some embodiments, Cy^{A} is an 8- to 12-membered bicyclic heteroarylene having 1-4 heteroatoms independently selected from oxygen, nitrogen, and sulfur, wherein Cy^{A} is substituted with 0-4-R^{A} groups. In some embodiments, Cy^{A} is a 9-membered bicyclic heteroarylene having 3-4 heteroatoms independently selected from oxygen and nitrogen, wherein Cy^{A} is substituted with 0-1-R^{A} groups. In some embodiments, Cy^{A} is a 9-membered bicyclic heteroarylene having 2 nitrogen heteroatoms, wherein Cy^{A} is substituted with 0-3-R^{A} groups. In some embodiments, Cy^{A} is a 9-membered bicyclic heteroarylene having 2 nitrogen heteroatoms, wherein Cy^{A} is substituted with 0-1-R^{A} groups. In some embodiments, Cy^{A} is benzoimidazolediyl substituted with 0-4 R^{A} groups. In some embodiments, Cy^{A} is a 10-membered bicyclic heteroarylene having 3-4 heteroatoms independently selected from oxygen and nitrogen, wherein Cy^{A} is substituted with 0-1-R^{A} groups.

In some embodiments, Cy^{A} is selected from the group consisting of:

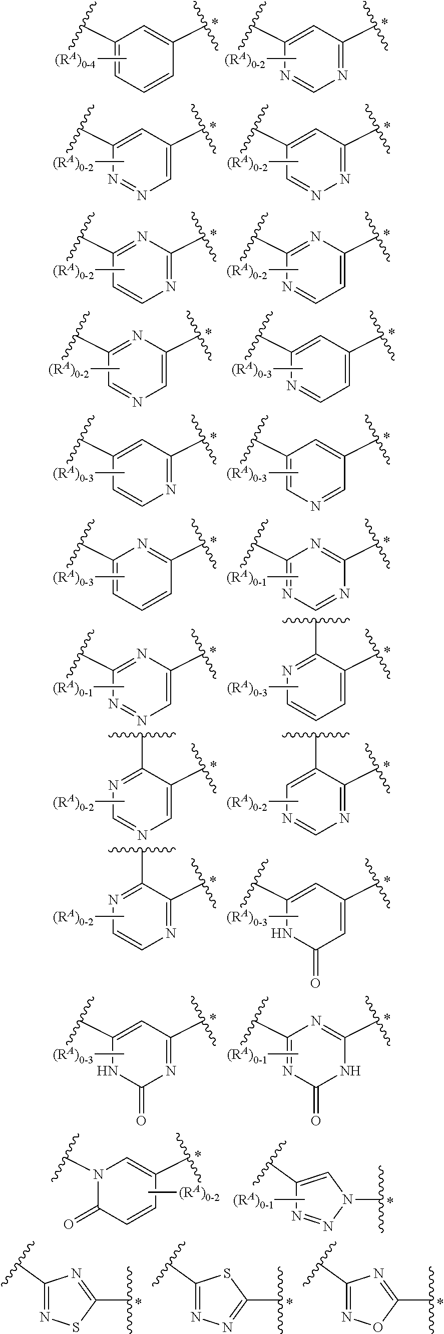

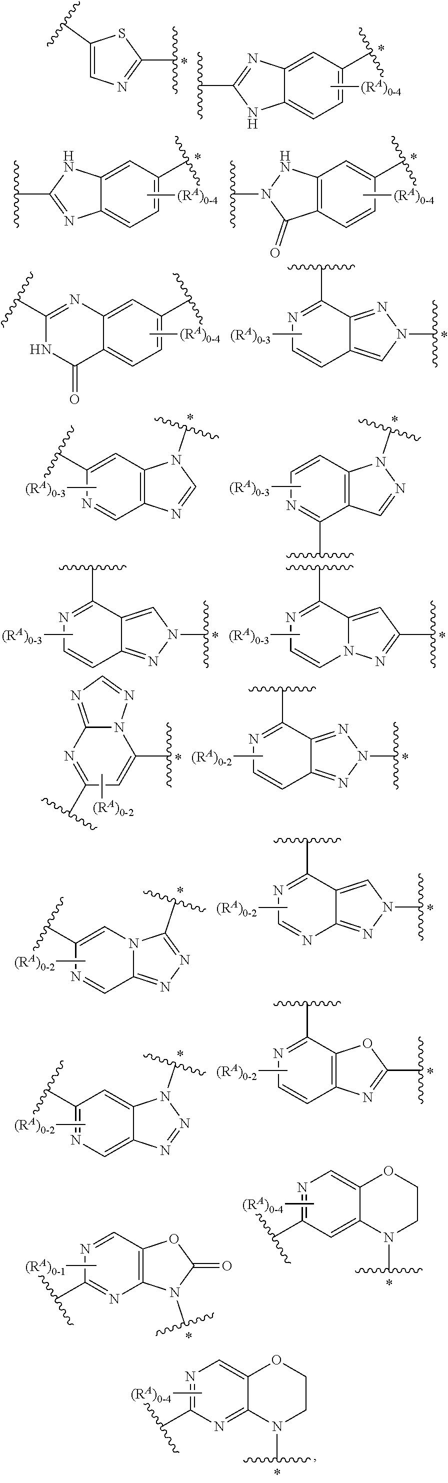

wherein * represents point of attachment to L.

In some embodiments, Cy^{A} is selected from the group consisting of:

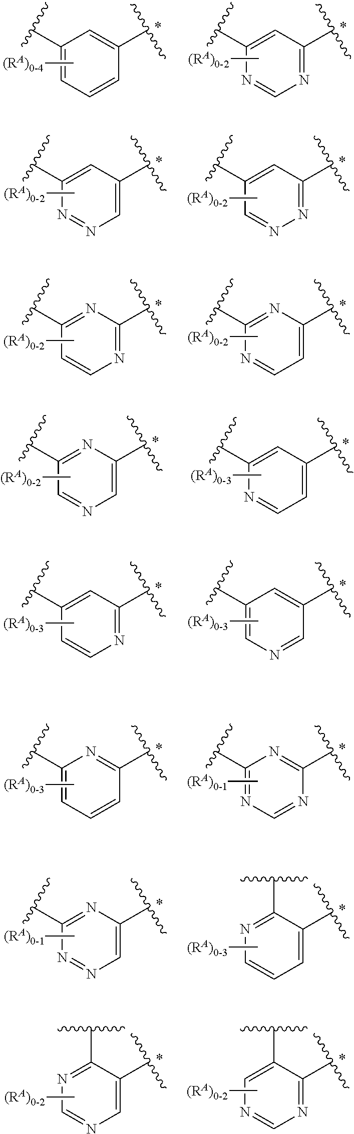

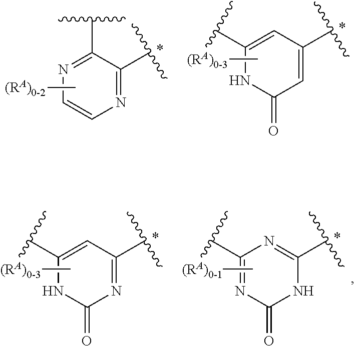

wherein * represents the point of attachment to L.

In some embodiments, Cy^{A} is selected from the group consisting of:

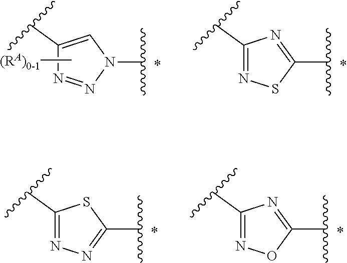

wherein * represents the point of attachment to L.

In some embodiments, Cy^{A} is selected from the group consisting of:

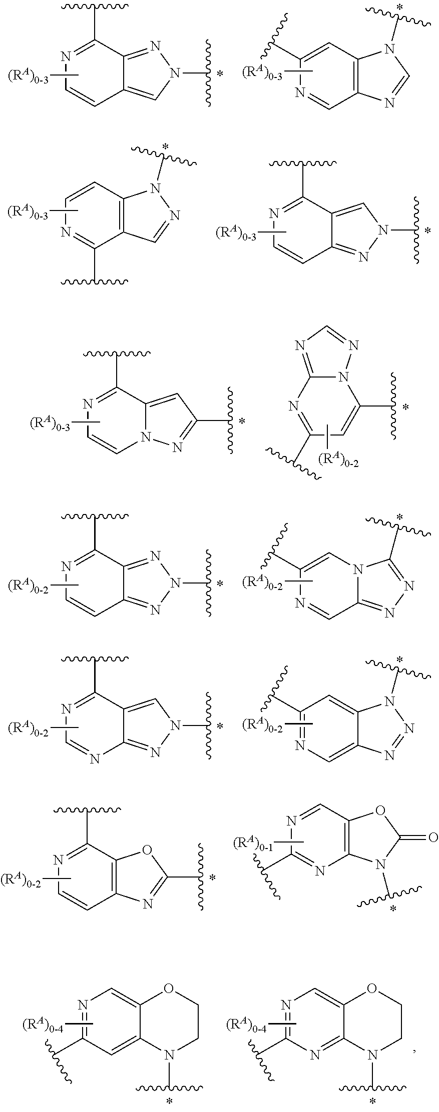

wherein * represents the point of attachment to L.

In some embodiments, Cy^{A} is selected from the group consisting of:

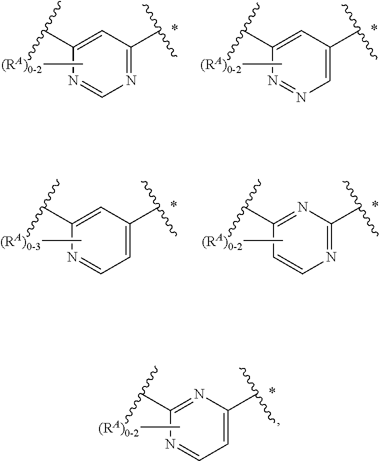

wherein * represents the point of attachment to L.

In some embodiments, Cy^{A} is selected from the group consisting of:

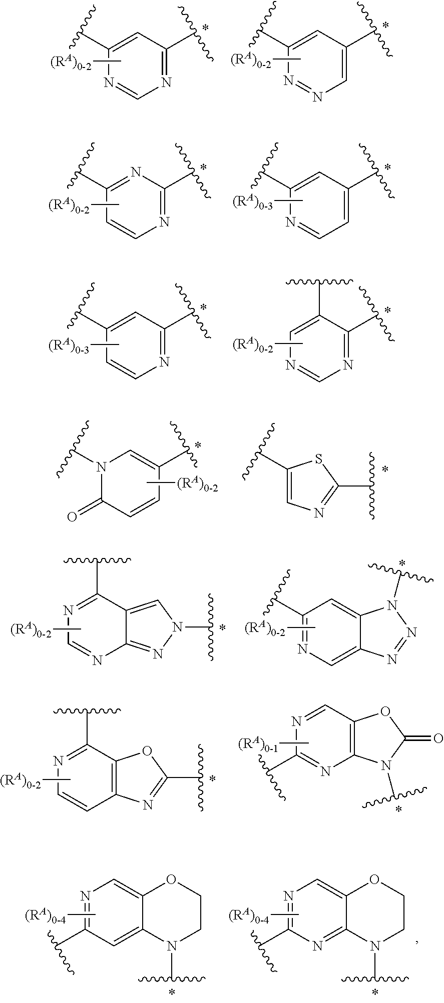

wherein * represents the point of attachment to L.

In some embodiments, Cy^{A} is selected from the group consisting of:

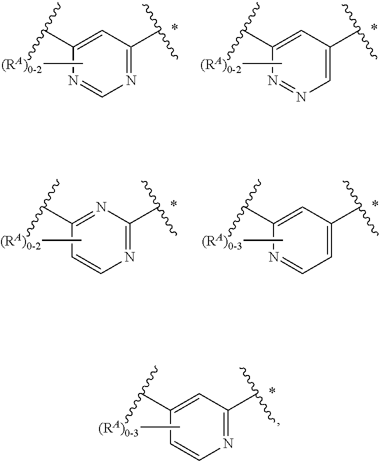

wherein represents the point of attachment to L.

In some embodiments, Cy^{A} is selected from the group consisting of:

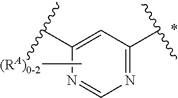

wherein * represents the point of attachment to L.

In some embodiments, Cy^{A} is selected from the group consisting of:

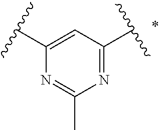 [SMILES: *c1cc(*)nc(C)n1]

wherein * represents the point of attachment to L.

In one embodiment, Cy^{A} comprising 0 R^{A} groups, i.e. Cy^{A} is unsubstituted.

In one embodiment, Cy^{A} comprises 1 R^{A} group, for example as described herein, in particular methyl.

In one embodiment, Cy^{A} comprises 2 R^{A} groups, for example independently selected from the groups/atoms described herein.

In some embodiments, each R^{A} is independently selected from oxo, halogen, —CN, —C(O)₂R, —N(R)₂, —OR, —SR, —S(O)R, —S(O)₂R, or an optionally substituted group selected from C₁₋₆ aliphatic, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, or sulfur.

In some embodiments, substituents on an optionally substituted R^{A} group are independently halogen, —(CH₂)₀₋₄OR^{o}, or —(CH₂)₀₋₄N(R^{o})₂, wherein each R^{o} is independently as defined above and described in classes and subclasses herein.

In some embodiments, each R^{A} is independently selected from oxo, —C(O)R, —C(O)₂R, —OR, or an optionally substituted group selected from C₁₋₆ aliphatic or a 5- to 6-membered monocyclic heteroaryl having 1-4 heteroatoms independently selected from oxygen, nitrogen or sulfur.

In some embodiments, substituents on an optionally substituted R^{A} group are independently halogen, —(CH₂)₀₋₄OR^{o}, or —(CH₂)₀₋₄C(O)OR^{o}, wherein each R^{o} is independently as defined above and described in classes and subclasses herein.

It will be appreciated that references herein to embodiments in which “a single instance” of a substituent is defined are not limited to monosubstituted embodiments. For example, “[i]n some embodiments, a single instance of R^{A} is oxo” includes embodiments in which at least one instance of R^{A} is oxo and which may comprise one or more additional R^{A} groups as defined herein.

In some embodiments, a single instance of R^{A} is oxo. In some embodiments, a single instance of R^{A} is halogen. In some embodiments, a single instance of R^{A} is —CN. In some embodiments, a single instance of R^{A} is —C(O)R. In some embodiments, a single instance of R^{A} is —C(O)Me. In some embodiments, a single instance of R^{A} is —C(O)₂R. In some embodiments, a single instance of R^{A} is —C(O)₂H. In some embodiments, a single instance of R^{A} is —C(O)₂Me. In some embodiments, a single instance of R^{A} is —C(O)₂Et. In some embodiments, a single instance of R^{A} is —N(R)₂. In some embodiments, a single instance of R^{A} is —OR. In some embodiments, a single instance of R^{A} is —OR, wherein R is optionally substituted C₁₋₆ aliphatic. In some embodiments, a single instance of R^{A} is —OR, wherein R is C₁₋₆ aliphatic optionally substituted with —(CH₂)₀₋₄OR^{o}, wherein each R^{o} is independently as defined above and described in classes and subclasses herein. In some embodiments, a single instance of R^{A} is —SR. In some embodiments, a single instance of R^{A} is —SR, wherein R is optionally substituted C₁₋₆ aliphatic. In some embodiments, a single instance of R^{A} is —S(O)R. In some embodiments, a single instance of R^{A} is —S(O)R, wherein R is optionally substituted C₁₋₆ aliphatic. In some embodiments, a single instance of R^{A} is —S(O)₂R. In some embodiments, a single instance of R^{A} is —S(O)₂R, wherein R is optionally substituted C₁₋₆ aliphatic.

In some embodiments, a single instance of R^{A} is optionally substituted C₁₋₆ aliphatic. In some embodiments, a single instance of R^{A} is C₁₋₆ aliphatic substituted with halogen. In some embodiments, a single instance of R^{A} is —CF₃. In some embodiments, a single instance of R^{A} is C₁₋₆-aliphatic substituted with —(CH₂)₀₋₄OR^{o}, wherein R^{o} is selected from hydrogen or C₁₋₆ aliphatic. In some embodiments, a single instance of R^{A} is C₁₋₆ aliphatic substituted with —(CH₂)₀₋₄N(R^{o})₂, wherein each R^{o} is independently selected from hydrogen or C₁₋₆ aliphatic. In some embodiments, a single instance of R^{A} is C₁₋₆ aliphatic substituted with —(CH₂)₀₋₄C(O)OR^{o}. In some embodiments, a single instance of R^{A} is selected from:

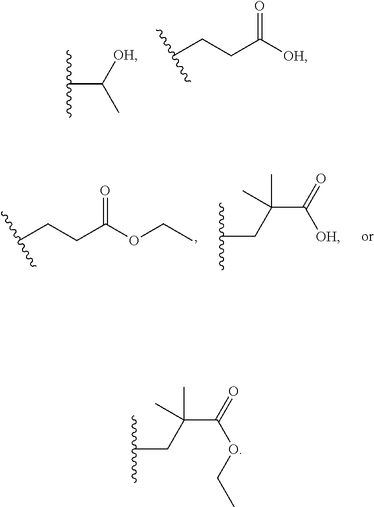

In some embodiments, a single instance of R^{A} is optionally substituted 3- to 7-membered saturated or partially unsaturated monocyclic carbocyclyl. In some embodiments, a single instance of R^{A} is optionally substituted cyclopropyl.

In some embodiments, a single instance of R^{A} is optionally substituted 3- to 7-membered saturated or partially unsaturated monocyclic heterocyclyl having 1-2 heteroatoms selected from oxygen, nitrogen, or sulfur. In some embodiments, a single instance of R^{A} is optionally substituted 3- to 7-membered saturated monocyclic heterocyclyl having 1-2 heteroatoms selected from oxygen and nitrogen. In some embodiments, a single instance of R^{A} is optionally substituted oxetanyl. In some embodiments, a single instance of R^{A} is oxetanyl optionally substituted with halogen or —(CH₂)₀₋₄OR^{o}. In some embodiments, a single instance of R^{A} is pyrrolidinyl.

In some embodiments, a single instance of R^{A} is optionally substituted 5- to 6-membered monocyclic heteroaryl having 1-4 heteroatoms independently selected from oxygen, nitrogen or sulfur. In some embodiments, a single instance of R^{A} is optionally substituted 5-membered monocyclic heteroaryl having 1-4 heteroatoms independently selected from oxygen, nitrogen or sulfur. In some embodiments, a single instance of R^{A} is optionally substituted 5-membered monocyclic heteroaryl having 1-4 nitrogen heteroatoms. In some embodiments, a single instance of R^{A} is optionally substituted tetrazolyl.

In some embodiments, Cy^{B} is selected from phenyl, a 5- to 6-membered heteroaryl having 1-3 heteroatoms independently selected from oxygen, nitrogen, and sulfur or a 7- to 10-membered heteroaryl having 1-4 heteroatoms independently selected from oxygen, nitrogen, and sulfur, wherein Cy^{B} is substituted with 0-4-R^{B} groups.

In some embodiments, Cy^{B} is selected from phenyl and a 5- to 6-membered heteroaryl having 1-3 heteroatoms independently selected from oxygen, nitrogen, and sulfur, wherein Cy^{B} is substituted with 0-4-R^{B} groups.

In some embodiments, Cy^{B} is selected from phenyl or a 5- to 6-membered heteroaryl having 1-3 heteroatoms independently selected from oxygen, nitrogen, and sulfur, wherein Cy^{B} is substituted with 0-4-R^{B} groups, for example pyrimidinyl substituted with 0-4-R^{B} groups, such as 0 or 1 group (in particular wherein 1 group is methyl).

In some embodiments, Cy^{B} is phenyl, wherein Cy^{B} is substituted with 0-5-R^{B} groups. In some embodiments, Cy^{B} is phenyl, wherein Cy^{B} is substituted with 0-3-R^{B} groups. In some embodiments, Cy^{B} is phenyl, wherein Cy^{B} is substituted with 0-2-R^{B} groups.

In some embodiments, Cy^{B} is a 6-membered heteroaryl having 1-3 heteroatoms independently selected from oxygen, nitrogen, and sulfur, wherein Cy^{B} is substituted with 0-4-R^{B} groups. In some embodiments, Cy^{B} is a 6-membered heteroaryl having 1-3 nitrogens, wherein Cy^{B} is substituted with 0-4-R^{B} groups. In some embodiments, Cy^{B} is a pyrimidinyl group substituted with 0-2 —R^{B} groups. In some embodiments, Cy^{B} is a pyridinyl group substituted with 0-2-R^{B} groups. In some embodiments, Cy^{B} is a pyrazinyl group substituted with 0-1-R^{B} groups. In some embodiments, Cy^{B} is a pyridazinyl group substituted with 0-1-R^{B} groups. In some embodiments, Cy^{B} is a 1,3,5-triazinyl group substituted with 0-1R^{B} groups. In some embodiments, Cy^{B} is a pyridinonyl group substituted with 0-1 additional R^{B} groups.

In some embodiments, Cy^{B} is a 5-membered heteroaryl having 1-3 heteroatoms independently selected from oxygen, nitrogen, and sulfur, wherein Cy^{B} is substituted with 0-4-R^{B} groups. In some embodiments, Cy^{B} is a 5-membered heteroaryl having 1-2 heteroatoms independently selected from sulfur and nitrogen, wherein Cy^{B} is substituted with 0-4-R^{B} groups. In some embodiments, Cy^{B} is a thienyl group substituted with 0-2-R^{B} groups. In some embodiments, Cy^{B} is a thiazolyl group substituted with 0-1R^{B} groups. In some embodiments, Cy^{B} is a thiadiazolyl group substituted with 0-1-R^{B} groups.

In some embodiments, Cy^{B} is selected from the group consisting of:

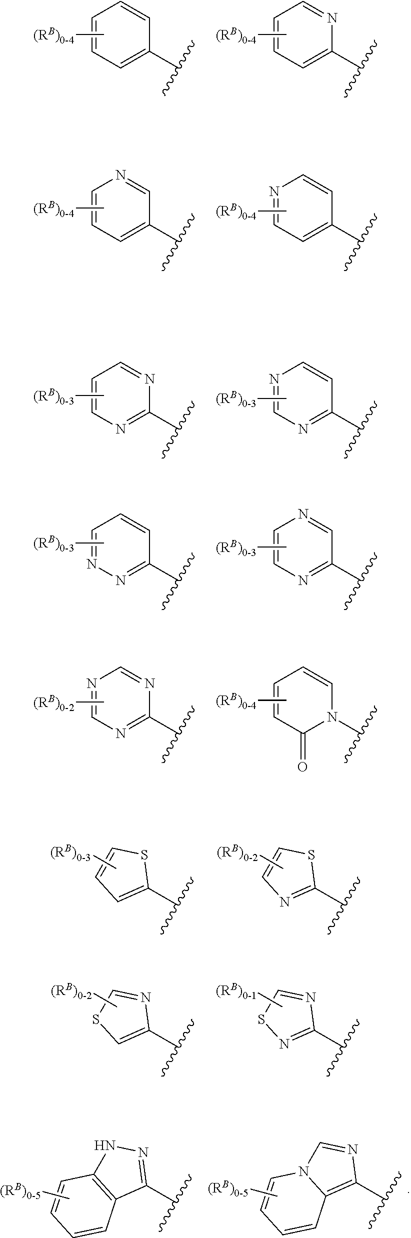

In some embodiments, Cy^{B} is selected from the group consisting of:

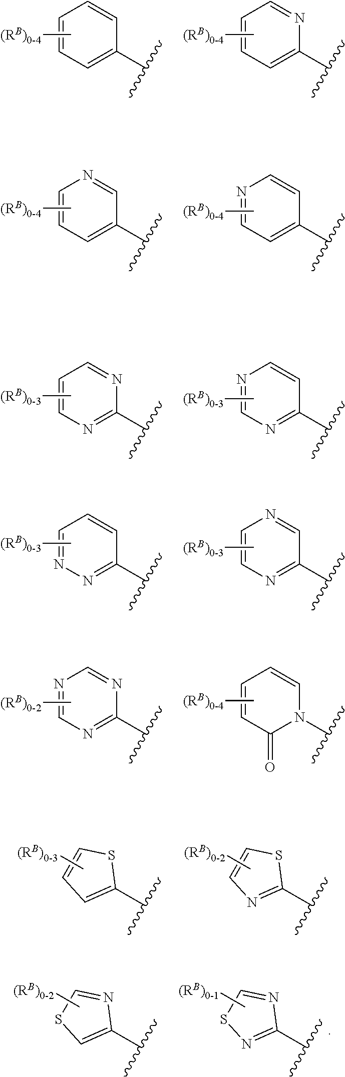

In some embodiments, Cy^{B} is selected from the group consisting of:

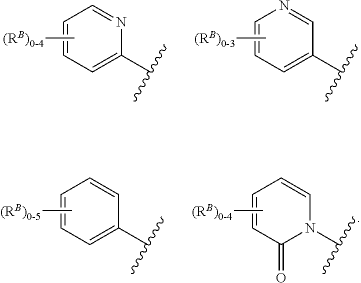

In some embodiments, Cy^{B} is:

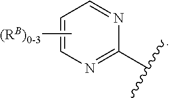

In some embodiments, Cy^{B} is a 8- to 10-membered bicyclic aryl, wherein Cy^{B} is substituted with 0-4-R^{B} groups. In some embodiments, Cy^{B} is a 10-membered bicyclic aryl, wherein Cy^{B} is substituted with 0-4-R^{B} groups. In some embodiments, Cy^{B} is a 1,2,3,4-tetrahydronaphthalenyl, wherein Cy^{B} is substituted with 0-4-R^{B} groups. In some embodiments, Cy^{B} is a naphthalenyl, wherein Cy^{B} is substituted with 0-4-R^{B} groups. In some embodiments, Cy^{B} is a indolyl, wherein Cy^{B} is substituted with 0-4-R^{B} groups.

In some embodiments, Cy^{B} is a 7- to 10-membered saturated or partially unsaturated bicyclic carbocycyl, wherein Cy^{B} is substituted with 0-4-R^{B} groups. In some embodiments, Cy^{B} is a 9-membered saturated or partially unsaturated bicyclic carbocycyl, wherein Cy^{B} is substituted with 0-4-R^{B} groups. In some embodiments, Cy^{B} is a 6,7-dihydro-5H-cyclopentapyridinyl, wherein Cy^{B} is substituted with 0-4-R^{B} groups.

In some embodiments, Cy^{B} is a 7- to 10-membered saturated or partially unsaturated bicyclic heterocycyl having 1-3 heteroatoms selected from oxygen, nitrogen, or sulfur, wherein Cy^{B} is substituted with 0-4-R^{B} groups. In some embodiments, Cy^{B} is a 9-membered saturated or partially unsaturated bicyclic heterocycyl having 1-3 heteroatoms selected from oxygen, nitrogen, or sulfur, wherein Cy^{B} is substituted with 0-4-R^{B} groups. In some embodiments, Cy^{B} is a 9-membered saturated or partially unsaturated bicyclic heterocycyl having 1-2 heteroatoms selected from oxygen or nitrogen, wherein Cy^{B} is substituted with 0-4-R^{B} groups. In some embodiments, Cy^{B} is a benzooxazolyl, wherein Cy^{B} is substituted with 0-4-R^{B} groups. In some embodiments, Cy^{B} is a benzooxazolonyl, wherein Cy^{B} is further substituted with 0-3 additional —R^{B} groups.

In some embodiments, Cy^{B} is a 7- to 10-membered heteroaryl having 1-4 heteroatoms independently selected from oxygen, nitrogen, and sulfur, wherein Cy^{B} is substituted with 0-4-R^{B} groups. In some embodiments, Cy^{B} is a 9-membered heteroaryl having 1-4 heteroatoms independently selected from oxygen, nitrogen, and sulfur, wherein Cy^{B} is substituted with 0-4-R^{B} groups. In some embodiments, Cy^{B} is a 9-membered heteroaryl having 1-3 nitrogen heteroatoms, wherein Cy^{B} is substituted with 0-4-R^{B} groups. In some embodiments, Cy^{B} is indoleyl, wherein Cy^{B} is substituted with 0-4-R^{B} groups. In some embodiments, Cy^{B} is imidazopyridinyl, wherein Cy^{B} is substituted with 0-4-R^{B} groups. In some embodiments, Cy^{B} is imidazopyridazinyl, wherein Cy^{B} is substituted with 0-4-R^{B} groups. In some embodiments, Cy^{B} is benzotriazolyl, wherein Cy^{B} is substituted with 0-4-R^{B} groups. In some embodiments, Cy^{B} is benzoimidazolyl, wherein Cy^{B} is substituted with 0-4-R^{B} groups. In some embodiments, Cy^{B} is pyrrolopyridinyl, wherein Cy^{B} is substituted with 0-4-R^{B} groups.

In some embodiments, Cy^{B} is a 10-membered heteroaryl having 1-4 heteroatoms independently selected from oxygen, nitrogen, and sulfur, wherein Cy^{B} is substituted with 0-4-R^{B} groups. In some embodiments, Cy^{B} is a 10-membered heteroaryl having 1-2 nitrogen heteroatoms, wherein Cy^{B} is substituted with 0-4-R^{B} groups. In some embodiments, Cy^{B} is quinazolinyl, wherein Cy^{B} is substituted with 0-4-R^{B} groups. In some embodiments, Cy^{B} is phthalazinyl, wherein Cy^{B} is substituted with 0-4-R^{B} groups.

In some embodiments, Cy^{B} is selected from the group consisting of:

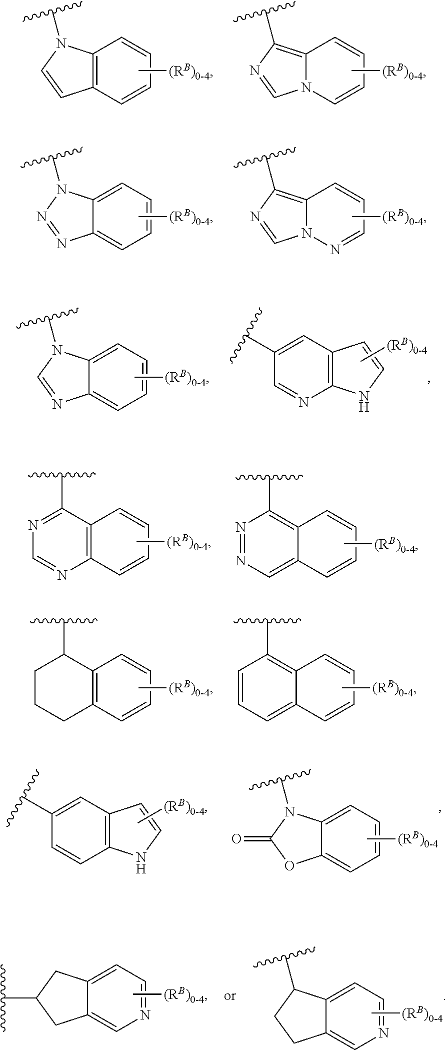

In some embodiments, Cy^{B} is selected from the group consisting of:

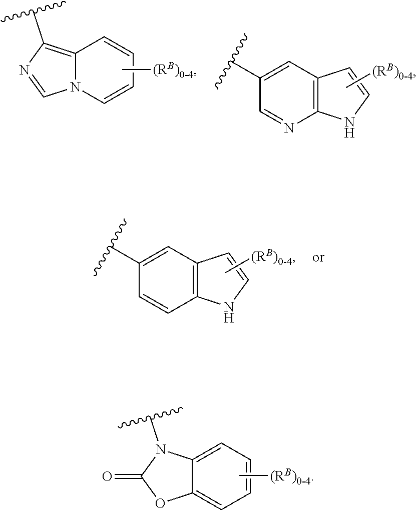

In some embodiments, Cy^{B} is

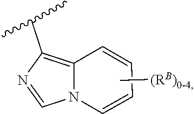

In some embodiments, each R^{B} is independently selected from oxo, halogen, —CN, —NO₂, —N(R)₂, —N(R)C(O)₂R, —OR, or an optionally substituted group selected from C₁₋₆ aliphatic or a 5-membered heteroaryl having 1-4 heteroatoms independently selected from oxygen, nitrogen, and sulfur.

In some embodiments, substituents on an optionally substituted R^{B} group are independently selected from oxo, halogen, and —(CH₂)₀₋₄OR^{o}, wherein R^{o} is as defined above and described in classes and subclasses herein.

In some embodiments, each R^{B} is independently selected from oxo, halogen, —CN, —C(O)N(R)₂, —C(NR)NR₂, —C(NR)NROR, —C(NR)NRC(O)OR, —N(R)₂, —OR, or an optionally substituted group selected from C₁₋₆ aliphatic, or a 5- to 6-membered heteroaryl having 1-4 heteroatoms independently selected from oxygen, nitrogen, and sulfur.

In some embodiments, substituents on an optionally substituted R^{B} group are independently selected from oxo, halogen, —(CH₂)₀₋₄OR^{o}, —(CH₂)₀₋₄N(R^{o})₂, —(CH₂)₀₋₄C(O)NR^{o}₂, and —(CH₂)₀₋₄OC(O)R^{o}; wherein R^{o} is as defined above and described in classes and subclasses herein.

In some embodiments, a single instance of R^{B} is oxo. In some embodiments, a single instance of R^{B} is halogen. In some embodiments, a single instance of R^{B} is —CN. In some embodiments, a single instance of R^{B} is —NO₂. In some embodiments, a single instance of R^{B} is —N(R)₂, In some embodiments, a single instance of R^{B} is —NH₂. In some embodiments, a single instance of R^{B} is —N(R)C(O)₂R. In some embodiments, a single instance of R^{B} is —OR. In some embodiments, a single instance of R^{B} is —OH. In some embodiments, a single instance of R^{B} is —OMe. In some embodiments, a single instance of R^{B} is —C(O)N(R)₂. In some embodiments, a single instance of R^{B} is —C(O)NH₂.

In some embodiments, a single instance of R^{B} is —C(NR)NR₂. In some embodiments, a single instance of R^{B} is —C(NH)NH₂. In some embodiments, a single instance of R^{B} is —C(NH)NHR, wherein R is an optionally substituted C₁₋₆ aliphatic. In some embodiments, a single instance of R^{B} is —C(NR)NRC(O)OR. In some embodiments, a single instance of R^{B} is —C(NH)NHC(O)OR. In some embodiments, a single instance of R^{B} is

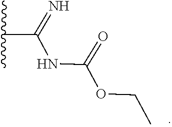 [SMILES: *C(=N)NC(=O)OCC]

In some embodiments, a single instance of R^{B} is

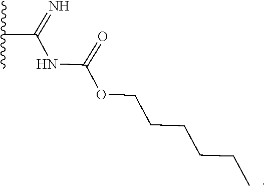 [SMILES: *C(=N)NC(=O)OCCCCCC]

In some embodiments, a single instance of R^{B} is —C(NR)NROR. In some embodiments, a single instance of R^{B} is —C(NH)NHOH. In some embodiments, a single instance of R^{B} is —C(NH)NHOR, wherein R is an optionally substituted C₁₋₆ aliphatic. In some embodiments, a single instance of R^{B} is —C(NH)NHOR, wherein R is C₁₋₆ aliphatic optionally substituted with —(CH₂)₀₋₄OC(O)R^{o}, wherein R^{o} is as defined above and described in classes and subclasses herein. In some embodiments, a single instance of R^{B} is

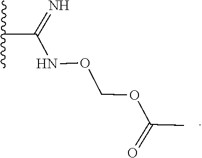 [SMILES: *C(=N)NOCOC(C)=O]

In some embodiments, a single instance of R^{B} is optionally substituted C₁₋₆ aliphatic. In some embodiments, a single instance of R^{B} is C₁₋₆ aliphatic substituted with halogen. In some embodiments, a single instance of R^{B} is

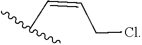 [SMILES: */C=C\CCl]

In some embodiments, a single instance of R^{B} is —CH₂NH₂. In some embodiments, a single instance of R^{B} is C₁₋₆ aliphatic substituted with —(CH₂)₀₋₄N(R^{o})₂. In some embodiments, a single instance of R^{B} is C₁₋₆ aliphatic substituted with —(CH₂)₀₋₄C(O)NR^{o}₂. In some embodiments, a single instance of R^{B} is —CH₂C(O)NH₂.

In some embodiments, a single instance of R^{B} is —N(R)C(O)₂R, wherein each R is independently selected from hydrogen or C₁₋₆ aliphatic optionally substituted with —(CH₂)₀₋₄R^{o}.

In some embodiments, a single instance of R^{B} is —OR, wherein each R is independently selected from hydrogen or C₁₋₆ aliphatic optionally substituted with halogen, —(CH₂)₀₋₄OR^{o}, or (CH₂)₀₋₄C(O)OR^{o}.

In some embodiments, a single instance of R^{B} is a 5-membered heteroaryl having 1-4 heteroatoms independently selected from oxygen, nitrogen, and sulfur. In some embodiments, a single instance of R^{B} is tetrazolyl.

In some embodiments, L is an optionally substituted C₁₋₂ hydrocarbon chain, wherein 1 methylene unit is optionally replaced with —C(O)—, —O—, —NR^{z}—; or L is an optionally substituted 5- to 6-membered saturated or partially unsaturated heterocyclene, having 1-3 heteroatoms independently selected from oxygen, nitrogen, and sulfur.

In some embodiments, L is —NR^{z}—.

In some embodiments, R^{z} is selected from H and C₁₋₆ aliphatic group, such as H or methyl, in particular methyl.

In some embodiments, L is —C(O)— or an optionally substituted C₂ hydrocarbon chain, wherein 1 methylene unit is optionally and independently replaced with —O— or —NR^{z}—; or L is an optionally substituted 5-membered saturated or partially unsaturated heterocyclene, having 1-3 heteroatoms independently selected from oxygen, nitrogen, and sulfur.

In some embodiments, L is an optionally substituted C₁₋₂ hydrocarbon chain, wherein 1 methylene unit is optionally replaced with —C(O)—, —O—, —NR^{z}—.

In some embodiments, L is an optionally substituted C₁ hydrocarbon chain. In some embodiments, L is an optionally substituted C₁ hydrocarbon chain, wherein 1 methylene unit is optionally replaced with —C(O)—, —O—, or —NR^{z}—. In some embodiments, L is —C(O)—. In some embodiments, L is a C₁ hydrocarbon chain, optionally substituted with halogen or —(CH₂)₀₋₄OR^{o}, wherein R^{o} is as defined above and described in classes and subclasses herein. In some embodiments, L is —CF₂—. In some embodiments, L is —C(OH)H—.

In some embodiments, L is an optionally substituted C₂ hydrocarbon chain, wherein 1 methylene unit is optionally replaced with —NR^{z}— or —O—. In some embodiments, L is an optionally substituted C₂ hydrocarbon chain, wherein the methylene unit connected to Cy^{A} is replaced with —NR^{z}- or —O—. In some embodiments, L is an optionally substituted C₂ hydrocarbon chain, wherein the methylene unit connected to Cy^{A} is replaced with —NR^{z}—. In some embodiments, L is an optionally substituted C₂ hydrocarbon chain, wherein the methylene unit connected to Cy^{A} is replaced with —O—. In some embodiments, L is optionally substituted *—NHCH₂—, wherein * represents the point of attachment to Cy^{A}. In some embodiments, L is optionally substituted *—OCH₂—, wherein * represents the point of attachment to Cy^{A}.

In some embodiments, L is *—NHCH(Me)-, wherein * represents the point of attachment to Cy^{A}. In some embodiments, L is *—NHCH₂—, wherein * represents the point of attachment to Cy^{A}. In some embodiments, L is *—OCH(Me)-, wherein * represents the point of attachment to Cy^{A}. In some embodiments, L is *—OCH₂— wherein * represents the point of attachment to Cy^{A}. In some embodiments, L is

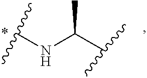 [SMILES: *N[C@H](*)C]

wherein * represents the point of attachment to Cy^{A}. In some embodiments, L is

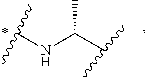 [SMILES: *N[C@@H](*)C]

wherein * represents the point of attachment to Cy^{A}. In some embodiments, L is *—N(CH)CH₂—, wherein * represents the point of attachment to Cy^{A}. In some embodiments, L is

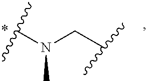

wherein * represents the point of attachment to Cy^{A}. In some embodiments, L is

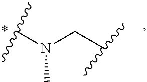

wherein * represents the point of attachment to Cy^{A}.

In some embodiments, L comprises a two-atom spacer between Cy^{A} and

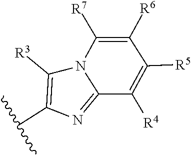

In some embodiments, L is an optionally substituted 5- to 6-membered saturated or partially unsaturated heterocyclene, having 1-3 heteroatoms independently selected from oxygen, nitrogen, and sulfur. In some embodiments, L is an optionally substituted 5-membered saturated or partially unsaturated heterocyclene, having 1 heteroatom independently selected from oxygen, nitrogen, and sulfur. In some embodiments, L is an optionally substituted pyrrolidinediyl group.

In some embodiments, L is optionally substituted

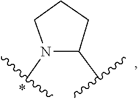 [SMILES: *C1CCCN1*]

wherein * represents the point of attachment to Cy^{A}. In some embodiments, L is optionally substituted

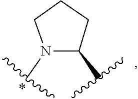 [SMILES: *[C@@H]1CCCN1*]

wherein * represents the point of attachment to Cy^{A}. In some embodiments, L is optionally substituted

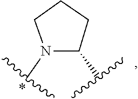 [SMILES: *[C@H]1CCCN1*]

wherein * represents the point of attachment to Cy^{A}. In some embodiments, L is

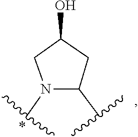 [SMILES: *C1C[C@H](O)CN1*]

wherein * represents the point of attachment to Cy^{A}. In some embodiments, L is

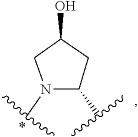 [SMILES: *[C@H]1C[C@H](O)CN1*]

wherein * represents the point of attachment to Cy^{A}. In some embodiments, L is

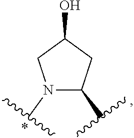 [SMILES: *[C@@H]1C[C@H](O)CN1*]

wherein * represents the point of attachment to Cy^{A}.

In some embodiments, optional substituents on L are independently selected from —(CH₂)₀₋₄R^{o}, —(CH₂)₀₋₄OR^{o}, —(CH₂)₀₋₄OC(O)R^{o}, and —(CH₂)₀₋₄N(R^{o})₂, wherein each R^{o} is independently as defined above and described in classes and subclasses herein.

In some embodiments, optional substituents on L are independently selected from halogen, —(CH₂)₀₋₄R^{o}, and —(CH₂)₀₋₄OR^{o}, wherein each R^{o} is independently as defined above and described in classes and subclasses herein.

In some embodiments, L′ is a covalent bond.

In some embodiments, L′ is an optionally substituted C₁₋₄ hydrocarbon chain, wherein 1 to 3 methylene units are optionally and independently replaced with —O—, —C(O)—, —NR^{z}—, —S—, —SO—, SO₂—, —S(NH)(O)—, or cyclopropylene. In some embodiments, L′ is an optionally substituted C₁₋₄ hydrocarbon chain, wherein 1 to 3 methylene units are optionally and independently replaced with —O—, —C(O)—, —NR^{z}—, SO₂—, —S(NH)(O)—, or cyclopropylene.

In some embodiments, L′ is an optionally substituted C₁ hydrocarbon chain, wherein the 1 methylene unit is optionally replaced with —O—, —C(O)—, or —NR^{z}—. In some embodiments, L′ is an optionally substituted C₁ hydrocarbon chain, wherein the 1 methylene unit is optionally replaced with —NR^{z}—. In some embodiments, L′ is —NH₂—. In some embodiments, L′ is an optionally substituted C₁ hydrocarbon chain. In some embodiments, L′ is —CH₂—.

In some embodiments, L′ is an optionally substituted C₂ hydrocarbon chain, wherein 1 to 2 methylene units are optionally and independently replaced with —O—, —C(O)—, or —NR^{z}—. In some embodiments, L′ is —CH₂CH₂—. In some embodiments, L′ is

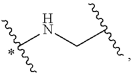 [SMILES: *CN*]

wherein * represents the point of attachment to Cy^{A}. In some embodiments, L′ is

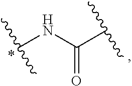 [SMILES: *NC(*)=O]

wherein * represents the point of attachment to Cy^{A}.

In some embodiments, L′ is an optionally substituted C₃ hydrocarbon chain, wherein 1 to 2 methylene units are optionally and independently replaced with —O—, —C(O)—, or —NR^{z}—. In some embodiments, L′ is a C₃ hydrocarbon chain, optionally substituted with —(CH₂)₀₋₄R^{o} or —(CH₂)₀₋₄OR^{o}, wherein 1 to 2 methylene units are optionally and independently replaced with —O—, —C(O)—, or —NR^{z}—. In some embodiments, L′ is an optionally substituted C₃ hydrocarbon chain, wherein 1 methylene unit is replaced with —C(O)—, and another methylene unit is replaced with —NR^{z}—. In some embodiments, L′ is selected from the group consisting of:

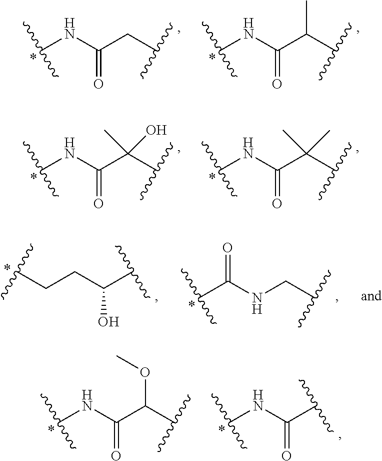

wherein * represents the point of attachment to Cy^{A}.

In some embodiments, L′ is selected from the group consisting of:

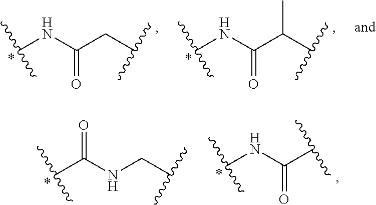

wherein * represents the point of attachment to Cy^{A}.

In some embodiments, L′ is an optionally substituted C₄ hydrocarbon chain, wherein 1 to 3 methylene units are optionally and independently replaced with —O—, —C(O)—, or —NR^{z}—. In some embodiments, L′ is a C₄ hydrocarbon chain, optionally substituted with —(CH₂)₀₋₄R^{o} or ═NR, wherein 1 to 3 methylene units are optionally and independently replaced with —O—, —C(O)—, or —NR^{z}—. In some embodiments, L′ is an optionally substituted C₄ hydrocarbon chain, wherein 1 methylene unit is replaced with —NR^{z}—, and 1 to 2 additional methylene units are optionally and independently replaced with —O—, —C(O)—, or —NR^{z}—. In some embodiments, L′ is an optionally substituted C₄ hydrocarbon chain, wherein 1 methylene unit is replaced with —NR^{z}—, 1 methylene unit is replaced with —C(O)—, and 1 methylene unity is optionally replaced with —O—, —C(O)—, or —NR^{z}—. In some embodiments, L′ is selected from the group consisting of:

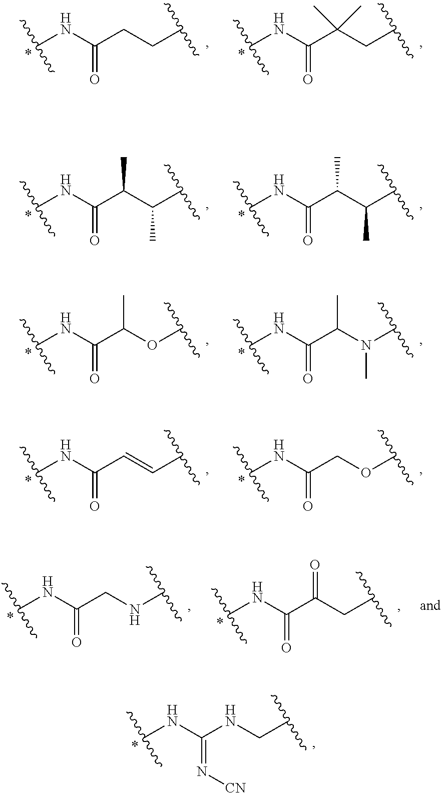

wherein * represents the point of attachment to Cy^{A}.

In some embodiments, L′ is an optionally substituted C₁₋₄ hydrocarbon chain, wherein 1 methylene unit is replaced with cyclopropylene, and 1 to 2 additional methylene units are optionally and independently replaced with —O—, —C(O)—, —NR—, SO₂—, or —S(NH)(O)—. It will be appreciated, that replacement of a single methylene unit of L′ with cyclopropylene may result in

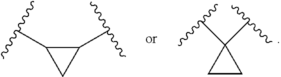 [SMILES: *C1CC1* ; *C1(*)CC1]

In some embodiments, L′ is an optionally substituted C₁₋₄ hydrocarbon chain, wherein 1 methylene unit of is replaced with cyclopropylene, and 1 or 2 additional methylene units are independently replaced with —O—, —C(O)—, —NR^{z}—, SO₂—, or —S(NH)(O)—. In some embodiments, L′ is selected from the group consisting of:

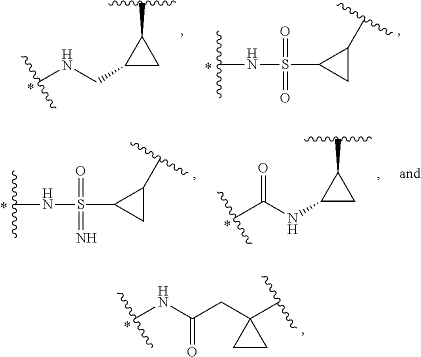 [SMILES: *NC[C@H]1C[C@@H]1* ; *NS(=O)(=O)C1CC1* ; *NS(=N)(=O)C1CC1* ; *C(=O)N[C@H]1C[C@@H]1* ; *NC(=O)CC1(*)CC1]

wherein * represents the point of attachment to Cy^{A}.

In some embodiments, each of R³, R⁴, R⁵, R⁶, and R⁷ is independently selected from hydrogen or L^{C}-R^{C}, wherein each L^{C} is independently selected from a covalent bond or an optionally substituted C₁₋₆ hydrocarbon chain, wherein 1 to 3 methylene units are optionally and independently replaced with —O— or —NR—; and wherein each R^{C} is independently selected from halogen, —CN, —C(O)R, —C(O)₂R, —C(O)N(R)₂, —N(R)₂, —N(R)C(O)R, —N(R)C(O)₂R, —N(R)S(O)₂R, —S(O)₂R, —S(O)₂N(R)₂, Cy^{C}, or an optionally substituted group selected from C₁₋₆ aliphatic.

In some embodiments each R^{Z} is hydrogen. In some embodiments each R^{Z} is independently selected from hydrogen, —(CH₂)₀₋₃OR, —(CH₂)₀₋₃C(O)OR, or an optionally substituted C₁₋₆ aliphatic group. In some embodiments each R^{z} is hydrogen or an optionally substituted C₁₋₆ aliphatic group. In some embodiments each R^{z} is hydrogen or a C₁₋₆ aliphatic group. In some embodiments R^{z} is is methyl.

In some embodiments, R³ is selected from hydrogen or L^{C}-R^{C}, wherein L^{C} is a covalent bond and R^{C} is halogen. In some embodiments, R³ is hydrogen. In some embodiments, R³ is L^{C}-R^{C}.

In some embodiments, R⁴ is selected from hydrogen or L^{C}-R^{C}, wherein L^{C} is selected from a covalent bond or an optionally substituted C₁₋₆ hydrocarbon chain, wherein 1 to 3 methylene units are optionally and independently replaced with —O— or —NR—; and wherein R^{C} is selected from halogen, —CN, —C(O)R, —C(O)₂R, —C(O)N(R)₂, —N(R)₂, —N(R)C(O)R, —N(R)C(O)₂R, —N(R)S(O)₂R, —OR, —S(O)₂R, —S(O)₂N(R)₂, Cy^{C}, or an optionally substituted group selected from C₁₋₆ aliphatic.

In some embodiments, R⁴ is selected from hydrogen or L^{C}-R^{C}, wherein L^{C} is a covalent bond and wherein R^{C} is selected from halogen, —CN, —C(O)R, —C(O)₂R, —C(O)N(R)₂, —N(R)₂, —N(R)C(O)R, —N(R)C(O)₂R, —N(R)S(O)₂R, —OR, —S(O)₂R, —S(O)₂N(R)₂, Cy^{C}, or an optionally substituted group selected from C₁₋₆ aliphatic.

In one some embodiment L^{C} is a covalent bond.

In some embodiments, R⁴ is hydrogen. In some embodiments, R⁴ is L^{C}-R^{C}. In some embodiments, R⁴ is L^{C}-R^{C}, wherein L^{C} is a covalent bond, and R^{C} is selected from —CN or Cy^{C}. In some embodiments, R⁴ is L^{C}-R^{C}, wherein L^{C} is a covalent bond, and R^{C} is Cy^{C}, wherein Cy^{C} is 3- to 7-membered saturated or partially unsaturated monocyclic heterocyclyl. In some embodiments, R⁴ is L^{C}-R^{C}, wherein L^{C} is an optionally substituted C₁₋₆ hydrocarbon chain, and R^{C} is —OR or —OC(O)R.

In some embodiments, R⁴ is selected from the group consisting of:

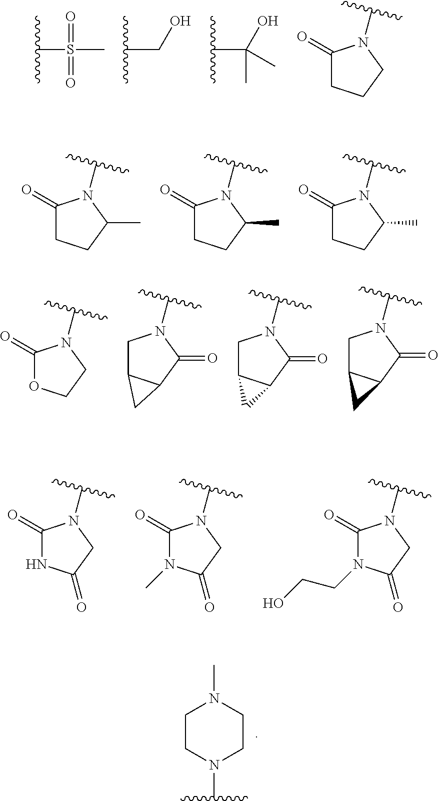

In some embodiments, R⁴ is selected from the group consisting of:

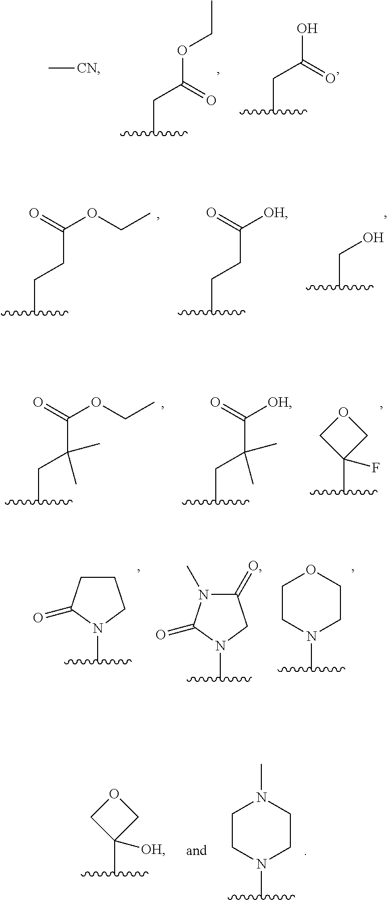

In some embodiments R⁴ is:

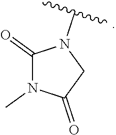 [SMILES: *N1CC(=O)N(C)C1=O]

In some embodiments of R⁴, optional substituents on a C₁₋₆aliphatic group are selected from —(CH₂)₀₋₄R^{o}, —(CH₂)₀₋₄OR^{o}, —CN, —(CH₂)₀₋₄N(R^{o})₂, and —(CH₂)₀₋₄C(O)OR^{o}, wherein each R^{o} is independently as defined above and described in classes and subclasses herein.

In some embodiments of R⁴, Cy^{C} is an optionally substituted group selected from a 3- to 7-membered saturated or partially unsaturated monocyclic heterocyclyl having 1-2 heteroatoms selected from oxygen, nitrogen, or sulfur, a 5- to 6-membered monocyclic heteroaryl having 1-3 heteroatoms independently selected from oxygen, nitrogen, and sulfur, a 6- to 12-membered saturated or partially unsaturated fused bicyclic heterocyclyl having 1-3 heteroatoms independently selected from oxygen, nitrogen, or sulfur, a bridged bicycle, or a 6- to 12-membered saturated or partially unsaturated bicyclic spiroheterocyclyl having 1-3 heteroatoms independently selected from oxygen, nitrogen, or sulfur. In some embodiments of R⁴, Cy^{C} is a 5-membered saturated or partially unsaturated monocyclic heterocyclyl having 1-2 heteroatoms selected from oxygen, nitrogen, or sulfur.

In some embodiments of R⁴, Cy^{C} is an optionally substituted group selected from the group consisting of:

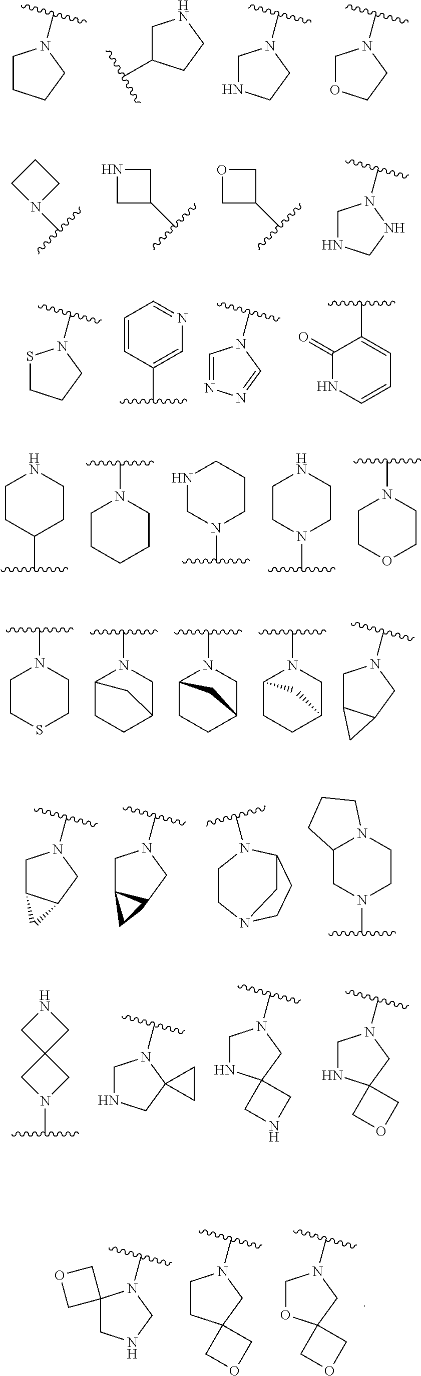

In some embodiments of R⁴, optional substituents on Cy are selected from halogen, —(CH₂)₀₋₄R^{o}, —(CH₂)₀₋₄OR^{o}, —(CH₂)₀₋₄N(R^{o})₂, —(CH₂)₀₋₄C(O)OR^{o}, and —OP(O)(OR^{o})₂, wherein each R^{o} is independently as defined above and described in classes and subclasses herein.

In some embodiments, R⁵ is hydrogen.

In some embodiments, R⁵ is L^{C}-R^{C}, wherein L^{C} is a covalent bond and R^{C} is Cy^{C}. In some embodiments, Cy^{C} is a cyclopropyl group.

In some embodiments, R⁶ is hydrogen. In some embodiments, R⁶ is selected from hydrogen or L^{C}-R^{C}, wherein L^{C} is a covalent bond, and wherein R^{C} is selected from halogen, —N(R)₂, —OR, Cy^{C}, or an optionally substituted C₁₋₆ aliphatic group. In some embodiments, R⁶ is L^{C}-R^{C}, wherein L^{C} is a covalent bond and R^{C} is Cy^{C}. In some embodiments of R⁶, Cy^{C} is an optionally substituted cyclopropyl. In some embodiments, R⁶ is cyclopropyl.

In some embodiments, R⁷ is selected from hydrogen or L^{C}-R^{C}, wherein L^{C} is a covalent bond, and wherein R^{C} is Cy^{C}. In some embodiments, R⁷ is hydrogen. In some embodiments, R⁷ is L^{C}-R^{C}, wherein L^{C} is a covalent bond and R^{C} is halogen. In some embodiments, R⁷ is fluorine. In some embodiments of R⁷, Cy^{C} is

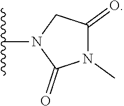 [SMILES: *N1CC(=O)N(C)C1=O]

In some embodiments, a provided compound is of Formula (I-a), Formula (I-b), or Formula (I-c):

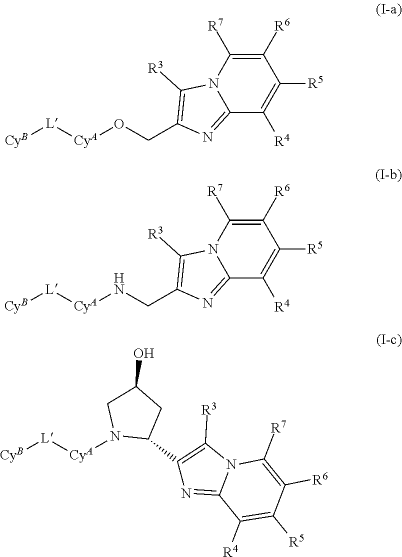

or a pharmaceutically acceptable salt thereof,
wherein each of Cy^{A}, Cy^{B}, L′, R³, R⁴, R⁵, R⁶, and R⁷ is defined and described in classes and subclasses herein, both singly and in combination.

It will be understood that, unless otherwise specified or prohibited by the foregoing definition of Formulae (I-a), (I-b), or (I-c), embodiments of variables Cy^{A}, Cy^{B}, L′, R³, R⁴, R⁵, R⁶, and R⁷ as defined above and described in classes and subclasses herein, also apply to compounds of Formulae (I-a), (I-b), or (I-c), both singly and in combination.

In some embodiments, a provided compound is of Formula (II), Formula (II-a), Formula (II-b), or Formula (II-c):

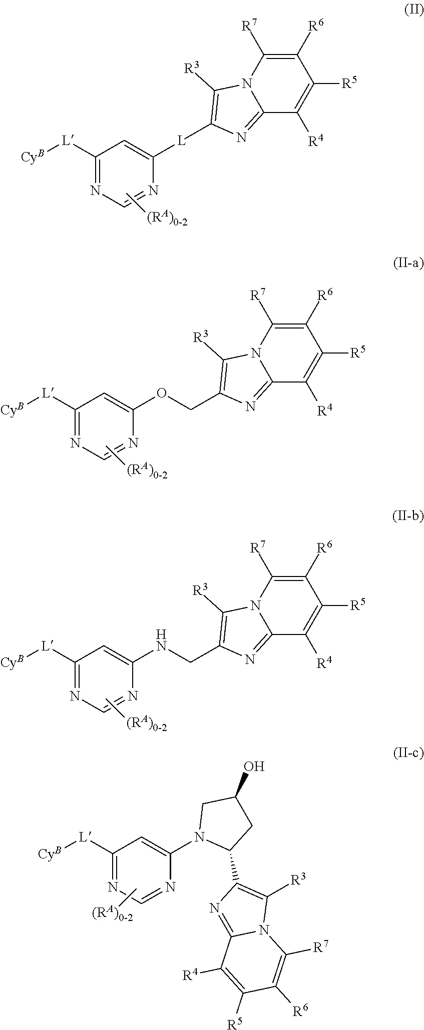

or a pharmaceutically acceptable salt thereof,
wherein each of R^{A}, Cy^{B}, L, L′, R³, R⁴, R⁵, R⁶, and R⁷ is defined and described in classes and subclasses herein, both singly and in combination.

It will be understood that, unless otherwise specified or prohibited by the foregoing definition of Formula (II), (II-a), (II-b), or (II-c), embodiments of variables R^{A}, Cy^{B}, L, L′, R³, R⁴, R⁵, R⁶, and R⁷ as defined above and described in classes and subclasses herein, also apply to compounds of Formula (II), (II-a), (II-b), or (II-c), both singly and in combination.

In some embodiments, a provided compound is of Formula (III), Formula (III-a), Formula (III-b), or Formula (III-c):

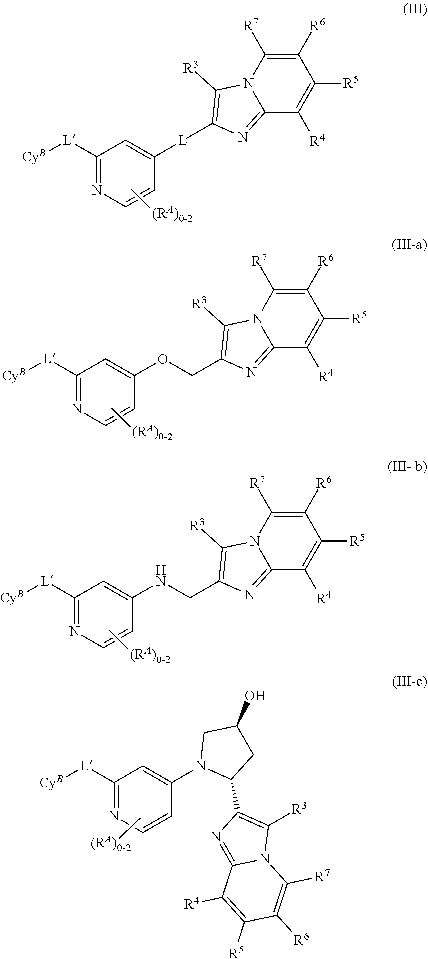

or a pharmaceutically acceptable salt thereof,
wherein each of R^{A}, Cy^{B}, L, L′, R³, R⁴, R⁵, R⁶, and R⁷ is defined and described in classes and subclasses herein, both singly and in combination.

It will be understood that, unless otherwise specified or prohibited by the foregoing definitions of Formulae (III), (III-a), (III-b), and (III-c), embodiments of variables R^{A}, Cy^{B}, L, L′, R³, R⁴, R⁵, R⁶, and R⁷ as defined above and described in classes and subclasses herein, also apply to compounds of Formulae (III), (III-a), (III-b), and (III-c), both singly and in combination.

In some embodiments, a provided compound is of Formula (IV-a), Formula (IV-b), Formula (IV-c), or Formula (IV-d):

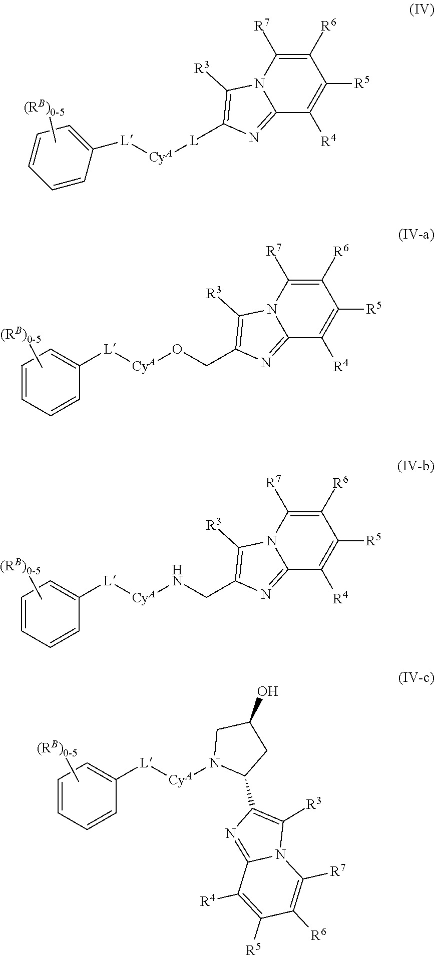

or a pharmaceutically acceptable salt thereof;
wherein each of Cy^{A}, R^{B}, L, L′, R³, R⁴, R⁵, R⁶, and R⁷ is defined and described in classes and subclasses herein, both singly and in combination.

It will be understood that, unless otherwise specified or prohibited by the foregoing definitions of Formulae (IV), (IV-a), (IV-b), (IV-c), embodiments of variables Cy^{A}, R^{B}, L, L′, R³, R⁴, R⁵, R⁶, and R⁷ as defined above and described in classes and subclasses herein, also apply to compounds of Formulae (IV), (IV-a), (IV-b), (IV-c), both singly and in combination.

In some embodiments, a provided compound is of Formula (V), (V-a), Formula (V-b), or Formula (V-c):

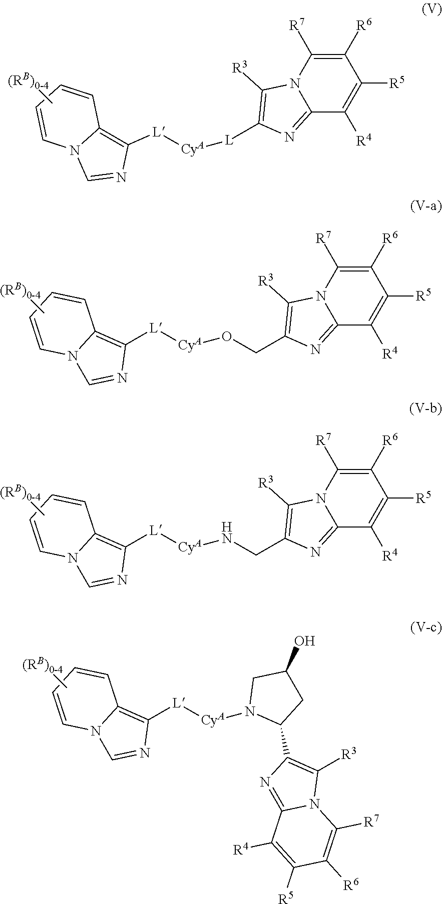

or a pharmaceutically acceptable salt thereof,
wherein each of Cy^{A}, R^{B}, L, L′, R³, R⁴, R⁵, R⁶, and R⁷ is defined and described in classes and subclasses herein, both singly and in combination.

It will be understood that, unless otherwise specified or prohibited by the foregoing definitions of Formulae (V), (V-a), (V-b), (V-c), embodiments of variables Cy^{A}, R^{B}, L, L′, R³, R⁴, R⁵, R⁶, and R⁷ as defined above and described in classes and subclasses herein, also apply to compounds of Formulae (V), (V-a), (V-b), (V-c), both singly and in combination.

In some embodiments, a provided compound is of Formula (VI), (VI-a), Formula (VI-b), or Formula (VI-c):

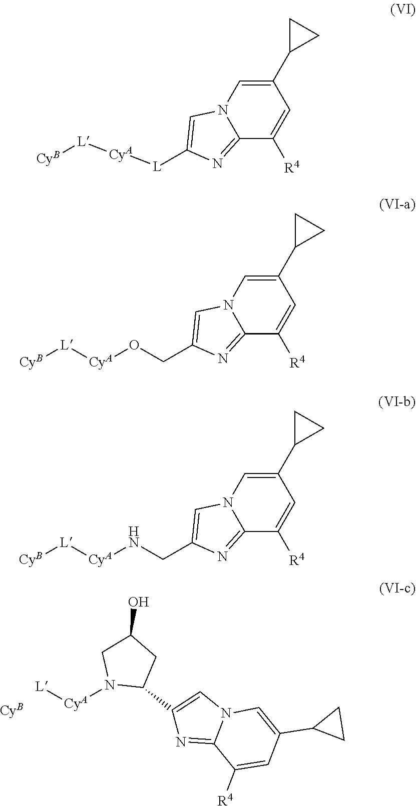

or a pharmaceutically acceptable salt thereof,
wherein each of Cy^{A}, Cy^{B}, L, L′, and R⁴ is defined and described in classes and subclasses herein, both singly and in combination.

It will be understood that, unless otherwise specified or prohibited by the foregoing definitions of Formulae (VI), (VI-a), (VI-b), and (VI-c), embodiments of variables Cy^{A}, Cy^{B}, L, L′, and R⁴ as defined above and described in classes and subclasses herein, also apply to compounds of Formulae (VI), (VI-a), (VI-b), and (VI-c), both singly and in combination.

In some embodiments, a provided compound is of Formula (VII), (VII-a), Formula (VII-b), or Formula (VII-c):

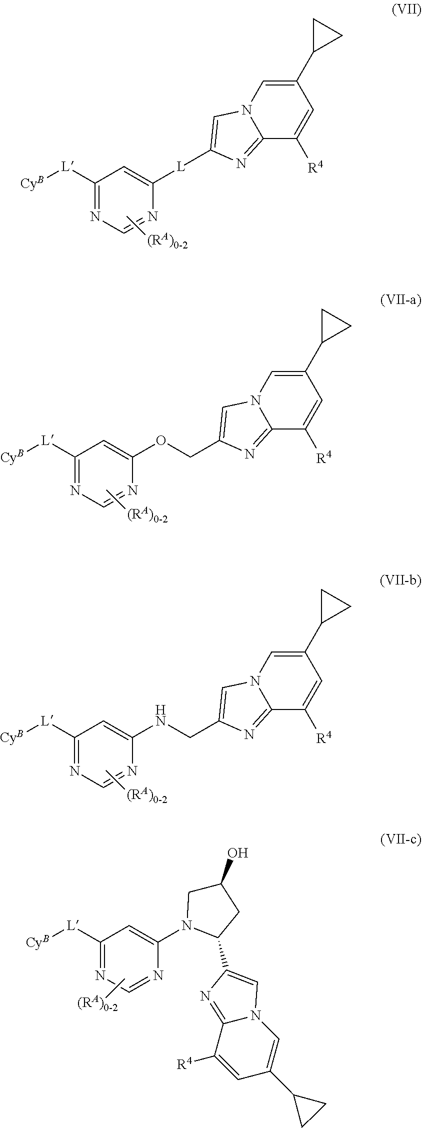

or a pharmaceutically acceptable salt thereof,
wherein each of R^{A}, Cy^{B}, L, L′, and R⁴ is defined and described in classes and subclasses herein, both singly and in combination.

It will be understood that, unless otherwise specified or prohibited by the foregoing definitions of Formulae (VII), (VII-a), (VII-b), and (VII-c), embodiments of variables R^{A}, Cy^{B}, L, L′, and R⁴ as defined above and described in classes and subclasses herein, also apply to compounds of Formulae (VII), (VII-a), (VII-b), and (VII-c), both singly and in combination.

In some embodiments, a provided compound is of Formula (VIII), (VIII-a), Formula (VIII-b), or Formula (VIII-c):

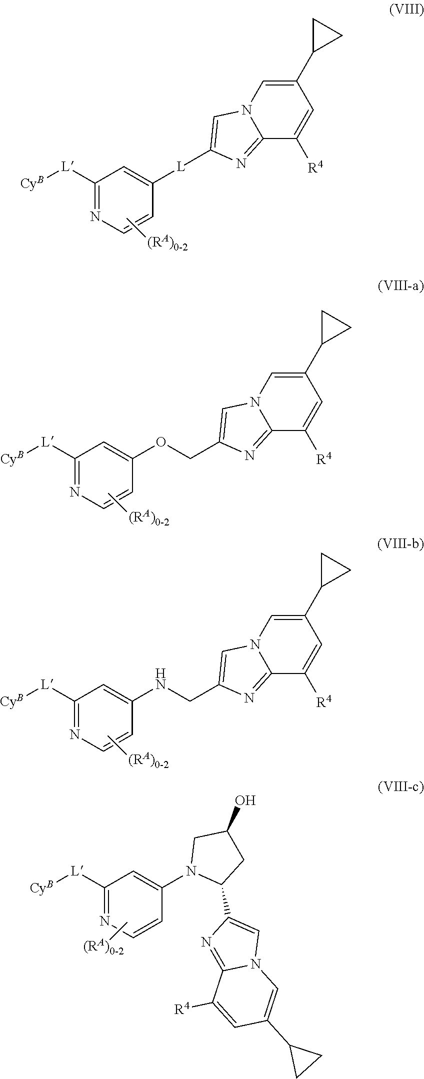

or a pharmaceutically acceptable salt thereof,
wherein each of R^{A}, Cy^{B}, L, L′, and R⁴ is defined and described in classes and subclasses herein, both singly and in combination.

It will be understood that, unless otherwise specified or prohibited by the foregoing definitions of Formulae (VIII), (VIII-a), (VIII-b), and (VIII-c), embodiments of variables R^{A}, Cy^{B}, L, L′, and R⁴ as defined above and described in classes and subclasses herein, also apply to compounds of Formulae (VIII), (VIII-a), (VIII-b), and (VIII-c), both singly and in combination.

In certain embodiments of provided compounds (i.e., of any species not otherwise defined and of any for Formula (I)-(VIII-c)), the moiety L′ may comprise a cyclopropyl ring:

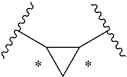 [SMILES: *C1CC1*]

where the relative trans configuration with respect to the bonds denoted [symbol] attached to the two stereocenters marked with an *. In other words, it will be appreciated that “trans” in the context of the moiety:

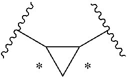 [SMILES: *C1CC1*]

is meant a compound comprising a mixture of

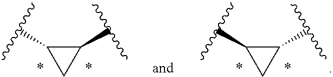 [SMILES: *[C@H]1C[C@@H]1* ; *[C@@H]1C[C@H]1*]

In some embodiments, such a mixture is a racemic mixture.

In certain embodiments of provided compounds (i.e., of any species not otherwise defined and of any of Formula (I)-(VIII-c)), where L′ comprises a cyclopropyl ring, the absolute stereochemistry of the moiety:

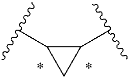 [SMILES: *C1CC1*]

is as follows:

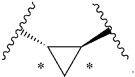 [SMILES: *[C@H]1C[C@@H]1*]

In certain embodiments of provided compounds (i.e., of any species not otherwise defined and of any of Formula (I)-(VIII-c)), where L′ comprises a cyclopropyl ring, the absolute stereochemistry of the moiety:

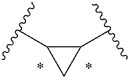 [SMILES: *C1CC1*]

is as follows:

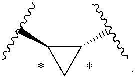 [SMILES: *[C@@H]1C[C@H]1*]

In some embodiments, the present invention provides a compound selected from:
2-(7-chloroimidazo[1,5-a]pyridin-1-yl)-N-(6-(((6-cyclopropylimidazo[1,2-a]pyridin-2-yl)methyl)amino)pyrimidin-4-yl)acetamide (I-1);
2-(5-chloro-2-cyanophenyl)-N-(6-(((6-cyclopropylimidazo[1,2-a]pyridin-2-yl)methyl)amino)pyrimidin-4-yl)acetamide (I-2);
2-(6-cyano-2-fluoro-3-methoxyphenyl)-N-(6-(((6-cyclopropylimidazo[1,2-a]pyridin-2-yl)methyl)amino)pyrimidin-4-yl)acetamide (I-3);
2-(7-chloroimidazo[1,5-a]pyridin-1-yl)-N-(6-(((6-cyclopropylimidazo[1,2-a]pyridin-2-yl)methyl)amino)pyrimidin-4-yl)propanamide (I-4);
2-(7-chloroimidazo[1,5-a]pyridin-1-yl)-N-(6-(((6-cyclopropylimidazo[1,2-a]pyridin-2-yl)methyl)amino)pyrimidin-4-yl)-2-methylpropanamide (I-5);
2-(7-chloroimidazo[1,5-a]pyridin-1-yl)-N-(6-(((6-cyclopropylimidazo[1,2-a]pyridin-2-yl)methyl)amino)pyrimidin-4-yl)-2-methoxyacetamide (I-6);
3-(3-chlorophenyl)-N-(6-(((6-cyclopropylimidazo[1,2-a]pyridin-2-yl)methyl)amino)pyrimidin-4-yl)-2-methylpropanamide (I-7);
3-(3-chlorophenyl)-N-(6-(((6-cyclopropylimidazo[1,2-a]pyridin-2-yl)methyl)amino)pyrimidin-4-yl)propanamide (I-8);
2-((3-chlorophenyl)(methyl)amino)-N-(6-(((6-cyclopropylimidazo[1,2-a]pyridin-2-yl)methyl)amino)pyrimidin-4-yl)propanamide (I-9);
2-(5-chloro-2-(1H-tetrazol-5-yl)phenyl)-N-(6-(((6-cyclopropylimidazo[1,2-a]pyridin-2-yl)methyl)amino)pyrimidin-4-yl)acetamide (I-10);
4-chloro-2-(2-((6-(((6-cyclopropylimidazo[1,2-a]pyridin-2-yl)methyl)amino)pyrimidin-4-yl)amino)-2-oxoethyl)benzamide (I-11);
2-(7-chloroimidazo[1,5-a]pyridin-1-yl)-N-(6-(((6-cyclopropyl-8-(2-oxopyrrolidin-1-yl)imidazo[1,2-a]pyridin-2-yl)methyl)amino)pyrimidin-4-yl)acetamide (I-12);
2-(7-chloroimidazo[1,5-a]pyridin-1-yl)-N-(6-(((8-cyano-6-cyclopropylimidazo[1,2-a]pyridin-2-yl)methyl)amino)pyrimidin-4-yl)acetamide (I-13);
2-(7-chloroimidazo[1,5-a]pyridin-1-yl)-N-(6-(((6-cyclopropyl-8-(3-hydroxyoxetan-3-yl)imidazo[1,2-a]pyridin-2-yl)methyl)amino)pyrimidin-4-yl)acetamide (I-14);
2-(5-chloro-2-(1H-tetrazol-1-yl)phenyl)-N-(6-(((8-cyano-6-cyclopropylimidazo[1,2-a]pyridin-2-yl)methyl)amino)pyrimidin-4-yl)acetamide (I-15);
2-(7-chloroimidazo[1,5-a]pyridin-1-yl)-N-(2-(((6-cyclopropylimidazo[1,2-a]pyridin-2-yl)methyl)amino)pyrimidin-4-yl)acetamide (I-16);
2-(7-chloroimidazo[1,5-a]pyridin-1-yl)-N-(6-(((6-cyclopropyl-8-(4-methylpiperazin-1-yl)imidazo[1,2-a]pyridin-2-yl)methyl)amino)pyrimidin-4-yl)acetamide (I-17);
2-(7-chloroimidazo[1,5-a]pyridin-1-yl)-N-(6-(((6-cyclopropyl-8-(2-oxopyrrolidin-1-yl)imidazo[1,2-a]pyridin-2-yl)methyl)(methyl)amino)pyrimidin-4-yl)acetamide (I-18);
2-(7-chloroimidazo[1,5-a]pyridin-1-yl)-N-(6-(((6-cyclopropyl-8-(3-fluorooxetan-3-yl)imidazo[1,2-a]pyridin-2-yl)methyl)amino)pyrimidin-4-yl)acetamide (I-19);
ethyl 4-(2-(7-chloroimidazo[1,5-a]pyridin-1-yl)acetamido)-6-(((6-cyclopropylimidazo[1,2-a]pyridin-2-yl)methyl)amino)pyrimidine-2-carboxylate (I-20);
4-(2-(7-chloroimidazo[1,5-a]pyridin-1-yl)acetamido)-6-(((6-cyclopropylimidazo[1,2-a]pyridin-2-yl)methyl)amino)pyrimidine-2-carboxylic acid (I-21);
2-(7-chloroimidazo[1,5-a]pyridin-1-yl)-N-(6-((8-cyano-6-cyclopropylimidazo[1,2-a]pyridin-2-yl)methoxy)pyrimidin-4-yl)acetamide (I-22);
2-(7-chloroimidazo[1,5-a]pyridin-1-yl)-N-(4-((6-cyclopropylimidazo[1,2-a]pyridin-2-yl)methoxy)pyridin-2-yl)acetamide (I-23);
N-(4-(((6-cyclopropyl-8-(2-oxopyrrolidin-1-yl)imidazo[1,2-a]pyridin-2-yl)methyl)amino)pyridin-2-yl)-2-(3-fluoro-4-methoxypyridin-2-yl)acetamide formic acid salt (I-24);
N-(4-(((6-cyclopropyl-8-(2-oxopyrrolidin-1-yl)imidazo[1,2-a]pyridin-2-yl)methyl)amino)pyridin-2-yl)-2-(3-fluoro-4-methoxypyridin-2-yl)acetamide;
2-(7-chloroimidazo[1,5-a]pyridin-1-yl)-N-(4-(((6-cyclopropylimidazo[1,2-a]pyridin-2-yl)methyl)amino)pyridin-2-yl)acetamide formic acid salt (I-25);
2-(7-chloroimidazo[1,5-a]pyridin-1-yl)-N-(4-(((6-cyclopropylimidazo[1,2-a]pyridin-2-yl)methyl)amino)pyridin-2-yl)acetamide;
2-(5-chloro-1H-indazol-3-yl)-N-(4-(((6-cyclopropylimidazo[1,2-a]pyridin-2-yl)methyl)amino)pyridin-2-yl)acetamide (I-26);
2-(6-chloro-1H-benzo[d][1,2,3]triazol-1-yl)-N-(4-(((6-cyclopropylimidazo[1,2-a]pyridin-2-yl)methyl)amino)pyridin-2-yl)acetamide (I-27);
2-(6-chloro-1H-benzo[d]imidazol-1-yl)-N-(4-(((6-cyclopropylimidazo[1,2-a]pyridin-2-yl)methyl)amino)pyridin-2-yl)acetamide (I-28);
1-amino-N-(4-(((6-cyclopropylimidazo[1,2-a]pyridin-2-yl)methyl)amino)pyridin-2-yl)-6,7-dihydro-5H-cyclopenta[c]pyridine-6-carboxamide (I-29);
2-(7-chloroimidazo[1,5-a]pyridin-1-yl)-N-(4-(((8-cyano-6-cyclopropylimidazo[1,2-a]pyridin-2-yl)methyl)amino)pyridin-2-yl)acetamide (I-30);
2-(7-chloroimidazo[1,5-a]pyridin-1-yl)-N-(5-(((6-cyclopropylimidazo[1,2-a]pyridin-2-yl)methyl)amino)pyridazin-3-yl)acetamide (I-31);
2-(7-chloroimidazo[1,5-a]pyridin-1-yl)-N-(5-((6-cyclopropylimidazo[1,2-a]pyridin-2-yl)methoxy)pyridazin-3-yl)acetamide (I-32);
2-(7-chloroimidazo[1,5-a]pyridin-1-yl)-N-(6-((6-cyclopropylimidazo[1,2-a]pyridin-2-yl)methoxy)pyrimidin-4-yl)acetamide (I-33);
2-(7-chloroimidazo[1,5-a]pyridin-1-yl)-N-(6-((6-cyclopropylimidazo[1,2-a]pyridin-2-yl)methoxy)pyridazin-4-yl)acetamide (I-34);
2-(5-chloro-2-(1H-tetrazol-1-yl)phenyl)-N-(6-(((6-cyclopropylimidazo[1,2-a]pyridin-2-yl)methyl)amino)pyrimidin-4-yl)acetamide (I-35);
2-(6-amino-2-fluoro-3-methoxyphenyl)-N-(6-(((6-cyclopropylimidazo[1,2-a]pyridin-2-yl)methyl)amino)pyrimidin-4-yl)acetamide (I-36);
N-(6-(((6-cyclopropylimidazo[1,2-a]pyridin-2-yl)methyl)amino)pyrimidin-4-yl)-2-(2-fluoro-3-methoxy-6-(1H-tetrazol-1-yl)phenyl)acetamide formic acid salt (I-37);
N-(6-(((6-cyclopropylimidazo[1,2-a]pyridin-2-yl)methyl)amino)pyrimidin-4-yl)-2-(2-fluoro-3-methoxy-6-(1H-tetrazol-1-yl)phenyl)acetamide;
ethyl 3-(2-(((6-(2-(7-chloroimidazo[1,5-a]pyridin-1-yl)acetamido)pyrimidin-4-yl)amino)methyl)-6-cyclopropylimidazo[1,2-a]pyridin-8-yl)propanoate (I-38);
3-(2-(((6-(2-(7-chloroimidazo[1,5-a]pyridin-1-yl)acetamido)pyrimidin-4-yl)amino)methyl)-6-cyclopropylimidazo[1,2-a]pyridin-8-yl)propanoic acid (I-39);
3-(2-(((6-(2-(7-chloroimidazo[1,5-a]pyridin-1-yl)acetamido)pyrimidin-4-yl)amino)methyl)-6-cyclopropylimidazo[1,2-a]pyridin-8-yl)-2,2-dimethylpropanoic acid (I-40);
2-(7-chloroimidazo[1,5-a]pyridin-1-yl)-N-(4-(((6-cyclopropyl-8-(hydroxymethyl)imidazo[1,2-a]pyridin-2-yl)methyl)amino)pyrimidin-5-yl)acetamide (I-41);
2-(7-chloroimidazo[1,5-a]pyridin-1-yl)-N-(4-(((6-cyclopropylimidazo[1,2-a]pyridin-2-yl)methyl)amino)pyrimidin-5-yl)acetamide (I-42);
2-(7-chloroimidazo[1,5-a]pyridin-1-yl)-N-(4-(((6-cyclopropyl-5-fluoroimidazo[1,2-a]pyridin-2-yl)methyl)amino)pyrimidin-5-yl)acetamide (I-43);
2-(7-chloroimidazo[1,5-a]pyridin-1-yl)-N-(6-(((6-cyclopropyl-8-(hydroxymethyl)imidazo[1,2-a]pyridin-2-yl)methyl)amino)pyrimidin-4-yl)acetamide formic acid salt (I-44);
2-(7-chloroimidazo[1,5-a]pyridin-1-yl)-N-(6-(((6-cyclopropyl-8-(hydroxymethyl)imidazo[1,2-a]pyridin-2-yl)methyl)amino)pyrimidin-4-yl)acetamide;
2-(7-chloroimidazo[1,5-a]pyridin-1-yl)-N-(6-((1-(6-cyclopropylimidazo[1,2-a]pyridin-2-yl)ethyl)amino)pyrimidin-4-yl)acetamide (I-45);
2-(7-chloroimidazo[1,5-a]pyridin-1-yl)-N-(6-(((6-cyclopropyl-5-fluoroimidazo[1,2-a]pyridin-2-yl)methyl)amino)pyrimidin-4-yl)acetamide (I-46);
ethyl 3-(2-(((6-(2-(7-chloroimidazo[1,5-a]pyridin-1-yl)acetamido)pyrimidin-4-yl)amino)methyl)-6-cyclopropylimidazo[1,2-a]pyridin-8-yl)-2,2-dimethylpropanoate (I-47);
2-(7-chloroimidazo[1,5-a]pyridin-1-yl)-N-(6-(((6-cyclopropyl-8-(morpholinomethyl)imidazo[1,2-a]pyridin-2-yl)methyl)amino)pyrimidin-4-yl)acetamide (I-48);
2-(3-chlorophenoxy)-N-(6-(((6-cyclopropylimidazo[1,2-a]pyridin-2-yl)methyl)amino)pyrimidin-4-yl)propanamide formic acid salt (I-49);
2-(3-chlorophenoxy)-N-(6-(((6-cyclopropylimidazo[1,2-a]pyridin-2-yl)methyl)amino)pyrimidin-4-yl)propanamide;
(E)-3-(3-chlorophenyl)-N-(6-(((6-cyclopropylimidazo[1,2-a]pyridin-2-yl)methyl)amino)pyrimidin-4-yl)acrylamide (I-50);
2-(2-bromo-5-chlorophenyl)-N-(6-(((6-cyclopropylimidazo[1,2-a]pyridin-2-yl)methyl)amino)pyrimidin-4-yl)acetamide (I-51);
2-(7-chloroimidazo[1,5-a]pyridin-1-yl)-N-(4-(((6-cyclopropyl-8-(hydroxymethyl)imidazo[1,2-a]pyridin-2-yl)methyl)amino)pyridin-2-yl)acetamide (I-52);
2-(7-bromo-8-fluoroimidazo[1,5-a]pyridin-1-yl)-N-(6-(((6-cyclopropylimidazo[1,2-a]pyridin-2-yl)methyl)amino)pyrimidin-4-yl)acetamide (I-53);
2-(7-chloroimidazo[1,5-a]pyridin-1-yl)-N-(6-(((6-cyclopropylimidazo[1,2-a]pyridin-2-yl)methyl)amino)pyrimidin-4-yl)-2-oxoacetamide (I-54);
N⁴-((6-cyclopropylimidazo[1,2-a]pyridin-2-yl)methyl)-N⁶-(2-fluoro-3-methoxy-6-(1H-tetrazol-1-yl)benzyl)pyrimidine-4,6-diamine (I-55);
N⁵-(6-(((6-cyclopropylimidazo[1,2-a]pyridin-2-yl)methyl)amino)pyrimidin-4-yl)-6,7-dihydro-5H-cyclopenta[c]pyridine-1,5-diamine (I-56);
2-(7-chloroimidazo[1,5-a]pyridin-1-yl)-N-(6-(6-cyclopropylimidazo[1,2-a]pyridine-2-carbonyl)pyrimidin-4-yl)acetamide (I-58);
6-((6-cyclopropylimidazo[1,2-a]pyridin-2-yl)difluoromethyl)-N-(2-fluoro-3-methoxy-6-(1H-tetrazol-1-yl)benzyl)pyrimidin-4-amine (I-62);
(6-cyclopropylimidazo[1,2-a]pyridin-2-yl)(6-((2-fluoro-3-methoxy-6-(1H-tetrazol-1-yl)benzyl)amino)pyrimidin-4-yl)methanol (I-63);
(6-(((6-amino-2,4-dimethylpyridin-3-yl)methyl)amino)pyrimidin-4-yl)(6-cyclopropylimidazo[1,2-a]pyridin-2-yl)methanol bis formic acid salt (I-64);
(6-(((6-amino-2,4-dimethylpyridin-3-yl)methyl)amino)pyrimidin-4-yl)(6-cyclopropylimidazo[1,2-a]pyridin-2-yl)methanol;
N-((7-chloroimidazo[1,5-a]pyridin-1-yl)methyl)-6-(((6-cyclopropylimidazo[1,2-a]pyridin-2-yl)methyl)amino)pyridazine-4-carboxamide (I-65);
N-((7-chloroimidazo[1,5-a]pyridin-1-yl)methyl)-6-((6-cyclopropylimidazo[1,2-a]pyridin-2-yl)methoxy)pyridazine-4-carboxamide (I-66);
2-(3-chlorophenyl)-N-(6-(((6-cyclopropylimidazo[1,2-a]pyridin-2-yl)methyl)amino)pyrimidin-4-yl)cyclopropane-1-sulfonamide (I-67);
3-(((7-chloroimidazo[1,5-a]pyridin-1-yl)methyl)amino)-4-(((6-cyclopropylimidazo[1,2-a]pyridin-2-yl)methyl)amino)cyclobut-3-ene-1,2-dione (I-68);
4-(((6-((6-chloroimidazo[1,2-a]pyridin-2-yl)methoxy)pyrimidin-4-yl)amino)methyl)-3,5-dimethylbenzimidamideq formic acid salt (I-69)
4-(((6-((6-chloroimidazo[1,2-a]pyridin-2-yl)methoxy)pyrimidin-4-yl)amino)methyl)-3,5-dimethylbenzimidamide;
ethyl 3-(4-((4-carbamimidoyl-2,6-dimethylbenzyl)amino)-6-((6-cyclopropylimidazo[1,2-a]pyridin-2-yl)methoxy)pyrimidin-2-yl)propanoate formic acid salt (I-70);
ethyl 3-(4-((4-carbamimidoyl-2,6-dimethylbenzyl)amino)-6-((6-cyclopropylimidazo[1,2-a]pyridin-2-yl)methoxy)pyrimidin-2-yl)propanoate;
4-(((6-((6-cyclopropylimidazo[1,2-a]pyridin-2-yl)methoxy)pyrimidin-4-yl)amino)methyl)-N-hydroxy-3,5-dimethylbenzimidamide (I-71);
4-(((6-((6-cyclopropylimidazo[1,2-a]pyridin-2-yl)methoxy)pyrimidin-4-yl)amino)methyl)-3,5-dimethylbenzimidamide formic acid salt (I-72);
4-(((6-((6-cyclopropylimidazo[1,2-a]pyridin-2-yl)methoxy)pyrimidin-4-yl)amino)methyl)-3,5-dimethylbenzimidamide;
ethyl 3-(4-((6-cyclopropylimidazo[1,2-a]pyridin-2-yl)methoxy)-6-((4-(N-hydroxycarbamimidoyl)-2,6-dimethylbenzyl)amino)pyrimidin-2-yl)propanoate (I-73);
3-(4-((4-carbamimidoyl-2,6-dimethylbenzyl)amino)-6-((6-cyclopropylimidazo[1,2-a]pyridin-2-yl)methoxy)pyrimidin-2-yl)propanoic acid formic acid salt (I-74);
3-(4-((4-carbamimidoyl-2,6-dimethylbenzyl)amino)-6-((6-cyclopropylimidazo[1,2-a]pyridin-2-yl)methoxy)pyrimidin-2-yl)propanoic acid;
ethyl 3-(4-((6-cyclopropylimidazo[1,2-a]pyridin-2-yl)methoxy)-6-((4-(N-((hexyloxy)carbonyl)carbamimidoyl)-2,6-dimethylbenzyl)amino)pyrimidin-2-yl)propanoate (I-75);
hexyl ((4-(((6-((6-cyclopropylimidazo[1,2-a]pyridin-2-yl)methoxy)pyrimidin-4-yl)amino)methyl)-3,5-dimethylphenyl)(imino)methyl)carbamate (I-76);
ethyl 3-(4-((6-cyclopropylimidazo[1,2-a]pyridin-2-yl)methoxy)-6-((4-(N-(ethoxycarbonyl)carbamimidoyl)-2,6-dimethylbenzyl)amino)pyrimidin-2-yl)propanoate (I-77);
3-(4-((6-cyclopropylimidazo[1,2-a]pyridin-2-yl)methoxy)-6-((4-(N-hydroxycarbamimidoyl)-2,6-dimethylbenzyl)amino)pyrimidin-2-yl)propanoic acid formic acid salt (I-78);
3-(4-((6-cyclopropylimidazo[1,2-a]pyridin-2-yl)methoxy)-6-((4-(N-hydroxycarbamimidoyl)-2,6-dimethylbenzyl)amino)pyrimidin-2-yl)propanoic acid;
3-(4-((6-cyclopropylimidazo[1,2-a]pyridin-2-yl)methoxy)-6-((4-(N-((hexyloxy)carbonyl)carbamimidoyl)-2,6-dimethylbenzyl)amino)pyrimidin-2-yl)propanoic acid (I-79);
3-(4-((6-cyclopropylimidazo[1,2-a]pyridin-2-yl)methoxy)-6-((4-(N-(ethoxycarbonyl)carbamimidoyl)-2,6-dimethylbenzyl)amino)pyrimidin-2-yl)propanoic acid (I-80);
ethyl 4-((6-cyclopropylimidazo[1,2-a]pyridin-2-yl)methoxy)-6-((4-(N-hydroxycarbamimidoyl)-2,6-dimethylbenzyl)amino)pyrimidine-2-carboxylate (I-81);
ethyl 4-((4-carbamimidoyl-2,6-dimethylbenzyl)amino)-6-((6-cyclopropylimidazo[1,2-a]pyridin-2-yl)methoxy)pyrimidine-2-carboxylate formic acid salt (I-82);
ethyl 4-((4-carbamimidoyl-2,6-dimethylbenzyl)amino)-6-((6-cyclopropylimidazo[1,2-a]pyridin-2-yl)methoxy)pyrimidine-2-carboxylate; ethyl ((4-(((6-((6-cyclopropylimidazo[1,2-a]pyridin-2-yl)methoxy)pyrimidin-4-yl)amino)methyl)-3,5-dimethylphenyl)(imino)methyl)carbamate (I-83);
4-((6-cyclopropylimidazo[1,2-a]pyridin-2-yl)methoxy)-6-((4-(N-hydroxycarbamimidoyl)-2,6-dimethylbenzyl)amino)pyrimidine-2-carboxylic acid (I-84);
4-((4-carbamimidoyl-2,6-dimethylbenzyl)amino)-6-((6-cyclopropylimidazo[1,2-a]pyridin-2-yl)methoxy)pyrimidine-2-carboxylic acid formic acid salt (I-85);
4-((4-carbamimidoyl-2,6-dimethylbenzyl)amino)-6-((6-cyclopropylimidazo[1,2-a]pyridin-2-yl)methoxy)pyrimidine-2-carboxylic acid;
ethyl 4-((6-cyclopropylimidazo[1,2-a]pyridin-2-yl)methoxy)-6-((4-(N-(ethoxycarbonyl)carbamimidoyl)-2,6-dimethylbenzyl)amino)pyrimidine-2-carboxylate (I-86);
ethyl 4-((6-cyclopropylimidazo[1,2-a]pyridin-2-yl)methoxy)-6-((4-(N-((hexyloxy)carbonyl)carbamimidoyl)-2,6-dimethylbenzyl)amino)pyrimidine-2-carboxylate (I-87);
4-((6-cyclopropylimidazo[1,2-a]pyridin-2-yl)methoxy)-6-((4-(N-(ethoxycarbonyl)carbamimidoyl)-2,6-dimethylbenzyl)amino)pyrimidine-2-carboxylic acid (I-88);
4-((6-cyclopropylimidazo[1,2-a]pyridin-2-yl)methoxy)-6-((4-(N-((hexyloxy)carbonyl)carbamimidoyl)-2,6-dimethylbenzyl)amino)pyrimidine-2-carboxylic acid (I-89);
((4-(((6-((6-cyclopropylimidazo[1,2-a]pyridin-2-yl)methoxy)pyrimidin-4-yl)amino)methyl)-3,5-dimethylbenzimidamido)oxy)methyl acetate (I-90);
4-(((6-((6-cyclopropylimidazo[1,2-a]pyridin-2-yl)methoxy)-2-(2H-tetrazol-5-yl)pyrimidin-4-yl)amino)methyl)-3,5-dimethylbenzimidamide formic acid salt (I-91);
4-(((6-((6-cyclopropylimidazo[1,2-a]pyridin-2-yl)methoxy)-2-(2H-tetrazol-5-yl)pyrimidin-4-yl)amino)methyl)-3,5-dimethylbenzimidamide;
3-(4-((4-carbamimidoyl-2,6-dimethylbenzyl)amino)-6-((6-cyclopropylimidazo[1,2-a]pyridin-2-yl)methoxy)pyrimidin-2-yl)-2,2-dimethylpropanoic acid formic acid salt (I-92);
3-(4-((4-carbamimidoyl-2,6-dimethylbenzyl)amino)-6-((6-cyclopropylimidazo[1,2-a]pyridin-2-yl)methoxy)pyrimidin-2-yl)-2,2-dimethylpropanoic acid;
ethyl 3-(4-((4-carbamimidoyl-2,6-dimethylbenzyl)amino)-6-((6-cyclopropylimidazo[1,2-a]pyridin-2-yl)methoxy)pyrimidin-2-yl)-2,2-dimethylpropanoate formic acid salt (I-93);
ethyl 3-(4-((4-carbamimidoyl-2,6-dimethylbenzyl)amino)-6-((6-cyclopropylimidazo[1,2-a]pyridin-2-yl)methoxy)pyrimidin-2-yl)-2,2-dimethylpropanoate;
ethyl 2-(2-(((6-((4-carbamimidoyl-2,6-dimethylbenzyl)amino)pyrimidin-4-yl)oxy)methyl)-6-cyclopropylimidazo[1,2-a]pyridin-8-yl)acetate formic acid salt (I-94);
ethyl 2-(2-(((6-((4-carbamimidoyl-2,6-dimethylbenzyl)amino)pyrimidin-4-yl)oxy)methyl)-6-cyclopropylimidazo[1,2-a]pyridin-8-yl)acetate;
2-(2-(((6-((4-carbamimidoyl-2,6-dimethylbenzyl)amino)pyrimidin-4-yl)oxy)methyl)-6-cyclopropylimidazo[1,2-a]pyridin-8-yl)acetic acid formic acid salt (I-95);
2-(2-(((6-((4-carbamimidoyl-2,6-dimethylbenzyl)amino)pyrimidin-4-yl)oxy)methyl)-6-cyclopropylimidazo[1,2-a]pyridin-8-yl)acetic acid;
ethyl 3-(2-(((6-((4-carbamimidoyl-2,6-dimethylbenzyl)amino)pyrimidin-4-yl)oxy)methyl)-6-cyclopropylimidazo[1,2-a]pyridin-8-yl)propanoate (I-96);
3-(2-(((6-((4-carbamimidoyl-2,6-dimethylbenzyl)amino)pyrimidin-4-yl)oxy)methyl)-6-cyclopropylimidazo[1,2-a]pyridin-8-yl)propanoic acid formic acid salt (I-97);
3-(2-(((6-((4-carbamimidoyl-2,6-dimethylbenzyl)amino)pyrimidin-4-yl)oxy)methyl)-6-cyclopropylimidazo[1,2-a]pyridin-8-yl)propanoic acid;
4-(((5-(((6-cyclopropylimidazo[1,2-a]pyridin-2-yl)methyl)amino)pyridazin-3-yl)amino)methyl)-3,5-dimethylbenzimidamide formic acid salt (I-98);
4-(((5-(((6-cyclopropylimidazo[1,2-a]pyridin-2-yl)methyl)amino)pyridazin-3-yl)amino)methyl)-3,5-dimethylbenzimidamide;
4-(((6-(((6-cyclopropylimidazo[1,2-a]pyridin-2-yl)methyl)amino)pyrimidin-4-yl)amino)methyl)-3,5-dimethylbenzimidamide formic acid salt (I-99);
4-(((6-(((6-cyclopropylimidazo[1,2-a]pyridin-2-yl)methyl)amino)pyrimidin-4-yl)amino)methyl)-3,5-dimethylbenzimidamide;
2-(7-chloroimidazo[1,5-a]pyridin-1-yl)-N-(6-(((6-cyclopropylimidazo[1,2-a]pyridin-2-yl)methyl)amino)-2-(2-methoxyethoxy)pyrimidin-4-yl)acetamide (I-100);
2-(7-chloroimidazo[1,5-a]pyridin-1-yl)-N-(6-(((6-cyclopropylimidazo[1,2-a]pyridin-2-yl)methyl)amino)-2-(trifluoromethyl)pyrimidin-4-yl)acetamide (I-101);
N-(2-acetyl-6-(((6-cyclopropylimidazo[1,2-a]pyridin-2-yl)methyl)amino)pyrimidin-4-yl)-2-(7-chloroimidazo[1,5-a]pyridin-1-yl)acetamide (I-102);
2-(7-chloroimidazo[1,5-a]pyridin-1-yl)-N-(6-(((6-cyclopropylimidazo[1,2-a]pyridin-2-yl)methyl)amino)-2-(1-hydroxyethyl)pyrimidin-4-yl)acetamide (I-103);
2-(6-chloroquinazolin-4-yl)-N-(6-(((6-cyclopropylimidazo[1,2-a]pyridin-2-yl)methyl)amino)pyrimidin-4-yl)acetamide (I-104);
2-(7-chlorophthalazin-1-yl)-N-(6-(((6-cyclopropylimidazo[1,2-a]pyridin-2-yl)methyl)amino)pyrimidin-4-yl)acetamide (I-105);
2-(7-chloro-8-fluoroimidazo[1,5-a]pyridin-1-yl)-N-(6-(((6-cyclopropylimidazo[1,2-a]pyridin-2-yl)methyl)amino)pyrimidin-4-yl)-2-hydroxypropanamide (I-106);
N-((6-amino-2,4-dimethylpyridin-3-yl)methyl)-6-((6-cyclopropylimidazo[1,2-a]pyridin-2-yl)methoxy)pyrimidin-4-amine (I-107);
N-(4-(aminomethyl)-2,6-dimethylbenzyl)-6-((6-cyclopropylimidazo[1,2-a]pyridin-2-yl)methoxy)pyrimidin-4-amine (I-108);
N-((6-amino-2,4-dimethylpyridin-3-yl)methyl)-6-((6-cyclopropyl-8-(3-fluorooxetan-3-yl)imidazo[1,2-a]pyridin-2-yl)methoxy)pyrimidin-4-amine (I-109);
5-(((4-((6-cyclopropylimidazo[1,2-a]pyridin-2-yl)methoxy)pyridin-2-yl)amino)methyl)-4,6-dimethylpyridin-2-amine (I-110);
N⁴-((6-amino-2,4-dimethylpyridin-3-yl)methyl)-N₆-((6-cyclopropylimidazo[1,2-a]pyridin-2-yl)methyl)pyrimidine-4,6-diamine (I-111);
2-(((6-(((6-amino-2,4-dimethylpyridin-3-yl)methyl)amino)pyrimidin-4-yl)oxy)methyl)-6-cyclopropylimidazo[1,2-a]pyridine-8-carbonitrile formic acid salt (I-112);
2-(((6-(((6-amino-2,4-dimethylpyridin-3-yl)methyl)amino)pyrimidin-4-yl)oxy)methyl)-6-cyclopropylimidazo[1,2-a]pyridine-8-carbonitrile;
(2-(((6-(((6-amino-2,4-dimethylpyridin-3-yl)methyl)amino)pyrimidin-4-yl)oxy)methyl)-6-cyclopropylimidazo[1,2-a]pyridin-8-yl)methanol formic acid salt (I-113);
(2-(((6-(((6-amino-2,4-dimethylpyridin-3-yl)methyl)amino)pyrimidin-4-yl)oxy)methyl)-6-cyclopropylimidazo[1,2-a]pyridin-8-yl)methanol;
(E)-3-(3-chlorophenyl)-N-(4-(((6-cyclopropylimidazo[1,2-a]pyridin-2-yl)methyl)amino)pyridin-2-yl)acrylamide (I-114);
2-(6-chloro-1H-indol-1-yl)-N-(4-(((6-cyclopropylimidazo[1,2-a]pyridin-2-yl)methyl)amino)pyridin-2-yl)acetamide (I-115);
2-(5-chloro-2-(1H-tetrazol-1-yl)phenoxy)-N-(4-(((6-cyclopropylimidazo[1,2-a]pyridin-2-yl)methyl)amino)pyridin-2-yl)acetamide (I-116);
2-((5-chloro-2-(1H-tetrazol-1-yl)phenyl)amino)-N-(4-(((6-cyclopropylimidazo[1,2-a]pyridin-2-yl)methyl)amino)pyridin-2-yl)acetamide (I-117);
7-chloro-N-(4-(((6-cyclopropylimidazo[1,2-a]pyridin-2-yl)methyl)amino)pyridin-2-yl)-2-naphthamide (I-118);
2-(5-chloro-2-oxopyridin-1 (2H)-yl)-N-(6-(((6-cyclopropylimidazo[1,2-a]pyridin-2-yl)methyl)amino)pyrimidin-4-yl)acetamide (I-119);
7-chloro-N-(4-(((6-cyclopropylimidazo[1,2-a]pyridin-2-yl)methyl)amino)pyridin-2-yl)-1,2,3,4-tetrahydronaphthalene-2-carboxamide (I-120);
N-(6-(((6-cyclopropylimidazo[1,2-a]pyridin-2-yl)methyl)amino)pyrimidin-4-yl)-2-(5-methoxy-2-oxopyridin-1 (2H)-yl)acetamide (I-121);
N-(6-(((6-cyclopropylimidazo[1,2-a]pyridin-2-yl)methyl)amino)pyrimidin-4-yl)-2-(5-methoxy-2-oxobenzo[d]oxazol-3 (2H)-yl)acetamide (I-122);
2-(5-chloro-2-oxobenzo[d]oxazol-3 (2H)-yl)-N-(6-(((6-cyclopropylimidazo[1,2-a]pyridin-2-yl)methyl)amino)pyrimidin-4-yl)acetamide (I-123);
2-(5-chloro-2-oxobenzo[d]oxazol-3 (2H)-yl)-N-(4-(((6-cyclopropylimidazo[1,2-a]pyridin-2-yl)methyl)amino)pyridin-2-yl)acetamide (I-124);
N-(4-(((6-cyclopropylimidazo[1,2-a]pyridin-2-yl)methyl)amino)pyridin-2-yl)-2-(5-methoxy-2-oxobenzo[d]oxazol-3 (2H)-yl)acetamide (I-125);
2-(1-(3-chlorophenyl)cyclopropyl)-N-(4-(((6-cyclopropylimidazo[1,2-a]pyridin-2-yl)methyl)amino)pyridin-2-yl)acetamide (I-126);
2-(3-chloro-1-(4-(((6-cyclopropylimidazo[1,2-a]pyridin-2-yl)methyl)amino)pyridin-2-yl)-6-fluoro-1H-indol-5-yl)acetamide (I-127);
(E)-1-(3-chlorobenzyl)-2-cyano-3-(4-(((6-cyclopropylimidazo[1,2-a]pyridin-2-yl)methyl)amino)pyridin-2-yl)guanidine (I-128);
2-(3-chloro-1H-pyrrolo[2,3-b]pyridin-5-yl)-N-(6-(((6-cyclopropylimidazo[1,2-a]pyridin-2-yl)methyl)amino)pyrimidin-4-yl)acetamide (I-129)
2-(3-chloro-1H-pyrrolo[2,3-b]pyridin-5-yl)-N-(4-(((6-cyclopropylimidazo[1,2-a]pyridin-2-yl)methyl)amino)pyridin-2-yl)acetamide (I-130);
2-(3-chloro-6-fluoro-1H-indol-5-yl)-N-(6-(((6-cyclopropylimidazo[1,2-a]pyridin-2-yl)methyl)amino)pyrimidin-4-yl)acetamide (I-131);
2-(3-chloro-6-fluoro-1H-indol-5-yl)-N-(4-(((6-cyclopropylimidazo[1,2-a]pyridin-2-yl)methyl)amino)pyridin-2-yl)acetamide (I-132);
(2S,3R)-3-(3-chlorophenyl)-N-(6-(((6-cyclopropylimidazo[1,2-a]pyridin-2-yl)methyl)amino)pyrimidin-4-yl)-2-methylbutanamide (I-133);
(2R,3S)-3-(3-chlorophenyl)-N-(6-(((6-cyclopropylimidazo[1,2-a]pyridin-2-yl)methyl)amino)pyrimidin-4-yl)-2-methylbutanamide (I-134);
(3S,5R)-1-(6-(((6-amino-2,4-dimethylpyridin-3-yl)methyl)amino)pyrimidin-4-yl)-5-(6-cyclopropylimidazo[1,2-a]pyridin-2-yl)pyrrolidin-3-ol (I-135);
N⁴-((6-cyclopropylimidazo[1,2-a]pyridin-2-yl)methyl)-N₆-(2,4-dimethoxybenzyl)pyrimidine-4,6-diamine (I-136);
(3S,5R)-1-(6-(((3-chloro-1H-pyrrolo[2,3-b]pyridin-5-yl)methyl)amino)pyrimidin-4-yl)-5-(6-cyclopropylimidazo[1,2-a]pyridin-2-yl)pyrrolidin-3-ol (I-137);
N⁴-(((1S,2S)-2-(3-chlorophenyl)cyclopropyl)methyl)-N₆-((6-cyclopropylimidazo[1,2-a]pyridin-2-yl)methyl)pyrimidine-4,6-diamine (I-138);
(3S,5R)-1-(6-(((3-chloro-6-fluoro-1H-indol-5-yl)methyl)amino)pyrimidin-4-yl)-5-(6-cyclopropylimidazo[1,2-a]pyridin-2-yl)pyrrolidin-3-ol (I-139);
(3S,5R)-5-(6-cyclopropylimidazo[1,2-a]pyridin-2-yl)-1-(6-(((6-fluoro-1H-indol-5-yl)methyl)amino)pyrimidin-4-yl)pyrrolidin-3-ol (I-140);
2-(3-chloroimidazo[1,5-b]pyridazin-5-yl)-N-(6-(((6-cyclopropyl-8-(3-methyl-2,4-dioxoimidazolidin-1-yl)imidazo[1,2-a]pyridin-2-yl)methyl)amino)pyrimidin-4-yl)acetamide (I-141); or
2-(3-chlorophenyl)-N-(6-(((6-cyclopropylimidazo[1,2-a]pyridin-2-yl)methyl)amino)pyrimidin-4-yl)cyclopropane-1-sulfonimidamide (I-142),
or a pharmaceutically acceptable salt thereof.

Compounds explicitly disclosed herein may be claimed as an individual compound, including where there is no reference to stereochemistry.

In some embodiments, the present invention provides a compound selected from:

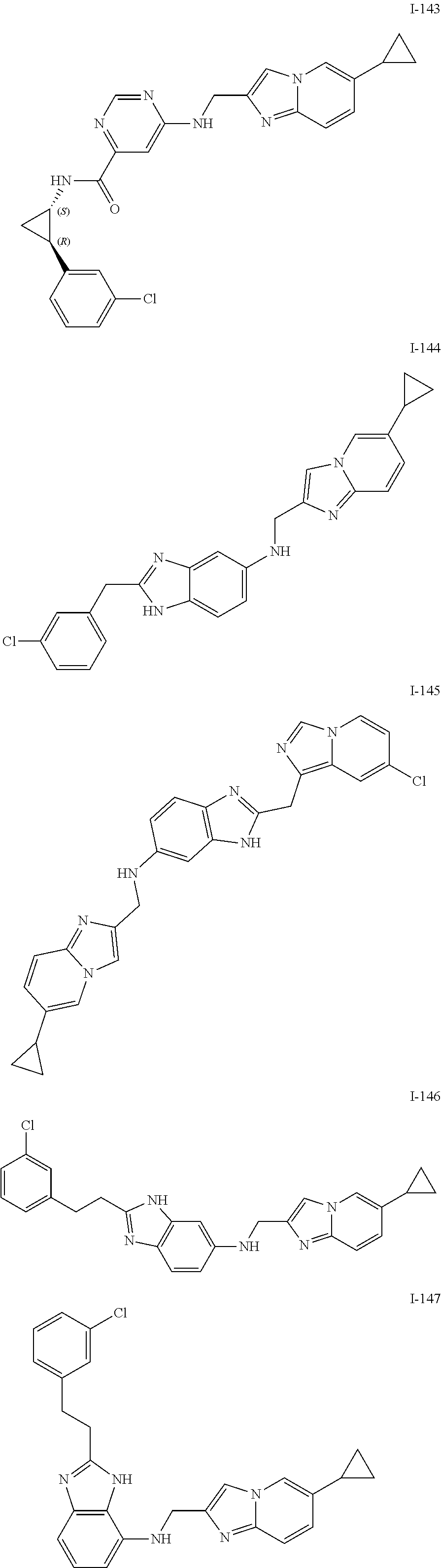

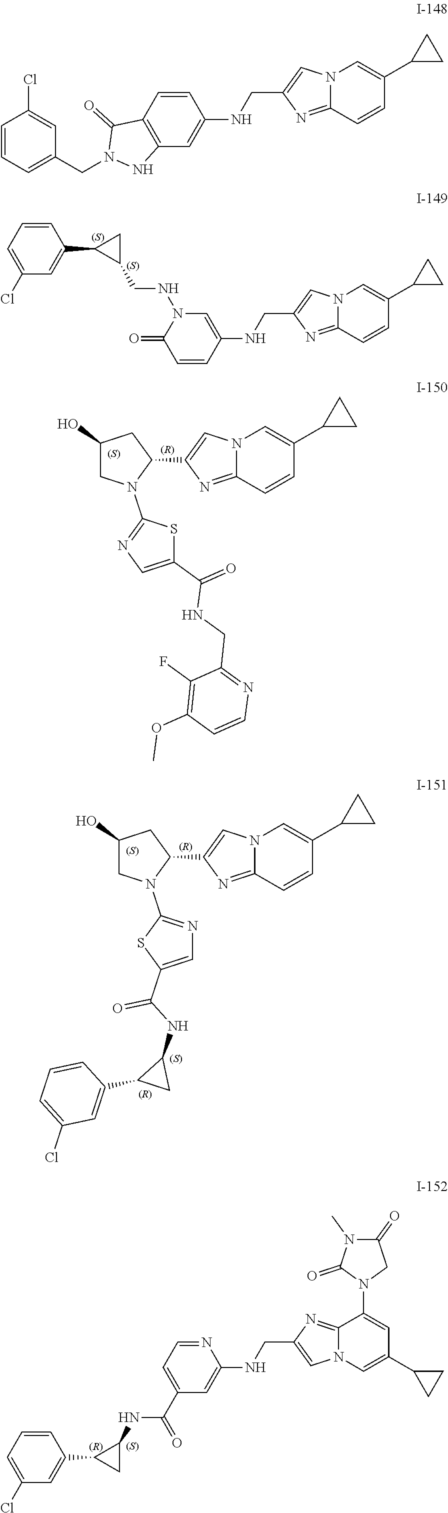

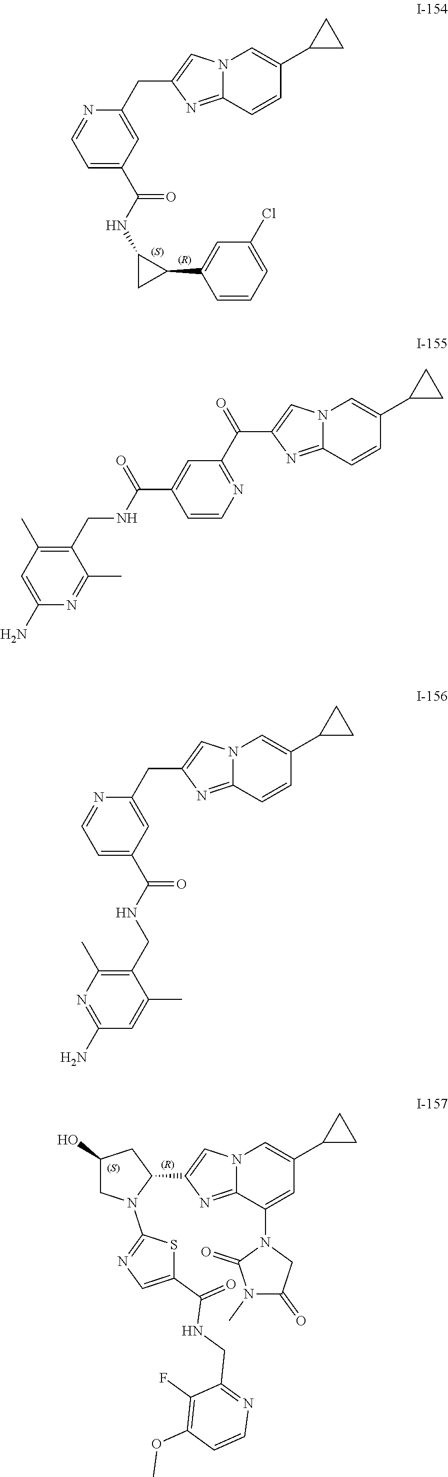

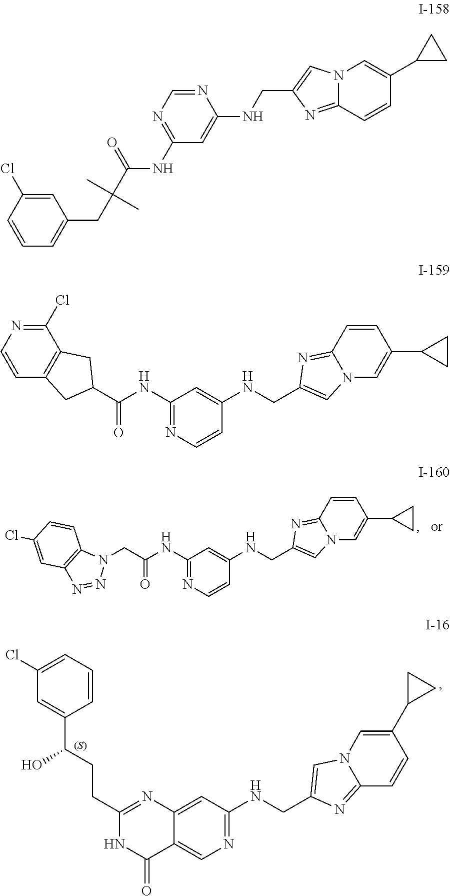

or pharmaceutically acceptable salt thereof.

In another aspect, the present invention provides a compound selected from:

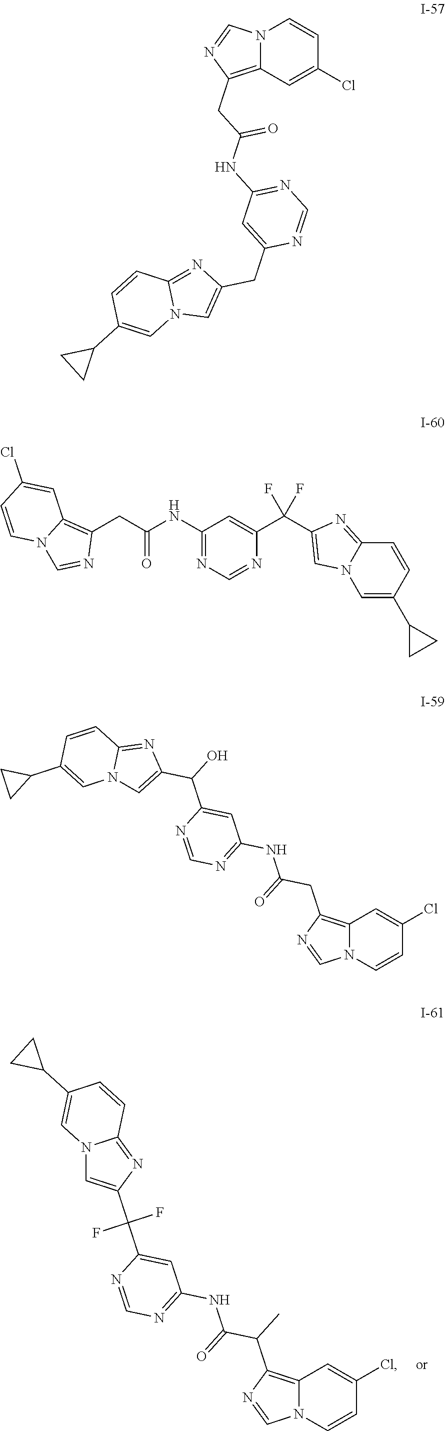

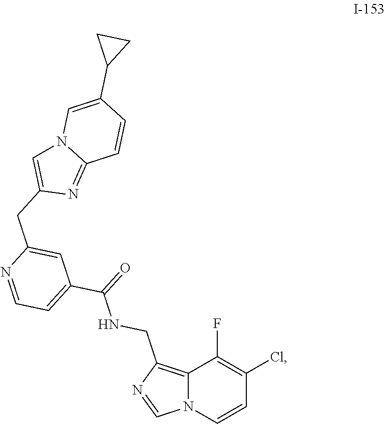 [SMILES: O=C(NCc1ncn2ccc(Cl)c(F)c12)c1ccnc(Cc2cn3cc(C4CC4)ccc3n2)c1]

or a pharmaceutically acceptable salt thereof.

In some embodiments, it will be appreciated that compounds I-1 through I-161 exhibit improvements when compared to a reference compound. In some embodiments, a reference compound is a PKa inhibitor known in the art. In some embodiments, a reference compound is a PKa inhibitor selected from those disclosed in PCT Publication Number WO 2019/178129.

In some embodiments, the present invention also provides methods of using compounds I-1 through I-161.

### C. Pharmaceutical Compositions

In another aspect, the present invention provides pharmaceutical compositions comprising a compound of the present disclosure, including Formulae (I)-(VIII-c) or compounds I-57, I-59 through I-61, or I-153 or a compound of Formulae (I)-(VIII-c) or compounds I-57, I-59 through I-61, or I-153 in combination with a pharmaceutically acceptable excipient (e.g., carrier).

The pharmaceutical compositions include optical isomers, diastereomers, or pharmaceutically acceptable salts of the inhibitors disclosed herein. A compound of Formulae (I)-(VIII-c) or compounds I-57, I-59 through I-61, or I-153 included in the pharmaceutical composition may be covalently attached to a carrier moiety, as described above. Alternatively, a compound of Formulae (I)-(VIII-c) or compounds I-57, I-59 through I-61, or I-153 included in the pharmaceutical composition is not covalently linked to a carrier moiety.

A “pharmaceutically acceptable carrier,” as used herein refers to pharmaceutical excipients, for example, pharmaceutically, physiologically, acceptable organic or inorganic carrier substances suitable for enteral or parenteral application that do not deleteriously react with the active agent. Suitable pharmaceutically acceptable carriers include water, salt solutions (such as Ringer's solution), alcohols, oils, gelatins, and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention.

The compounds of the invention can be administered alone or can be coadministered to the subject. Coadministration is meant to include simultaneous or sequential administration of the compounds individually or in combination (more than one compound). The preparations can also be combined, when desired, with other active substances (e.g. to reduce metabolic degradation).

In some embodiments, a compound as described herein can be incorporated into a pharmaceutical composition for administration by methods known to those skilled in the art and described herein for provided compounds.

### D. Formulations

Compounds of the present invention can be prepared and administered in a wide variety of oral, parenteral, and topical dosage forms. Thus, the compounds of the present invention can be administered by injection (e.g. intravenously, intramuscularly, intracutaneously, subcutaneously, intraduodenally, or intraperitoneally). In some embodiments compounds of the present disclosure are administered orally. Also, the compounds described herein can be administered by inhalation, for example, intranasally. Additionally, the compounds of the present invention can be administered transdermally. It is also envisioned that multiple routes of administration (e.g., intramuscular, oral, transdermal) can be used to administer the compounds of the invention. Accordingly, the present invention also provides pharmaceutical compositions comprising a pharmaceutically acceptable carrier or excipient and one or more compounds of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substance that may also act as diluents, flavoring agents, binders, preservatives, tablet disintegrating agents, or an encapsulating material.

In powders, the carrier is a finely divided solid in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The compounds of the invention can also be incorporated into liposomes or administered via transdermal pumps or patches. Pharmaceutical admixtures suitable for use in the present invention include those described, for example, in Pharmaceutical Sciences (17th Ed., Mack Pub. Co., Easton, PA) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The compositions of the present invention may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

### E. Effective Dosages

Pharmaceutical compositions provided by the present invention include compositions wherein the active ingredient is contained in a therapeutically effective amount, i.e., in an amount effective to achieve its intended purpose. The actual amount effective for a particular application will depend, inter alia, on the condition being treated. For example, when administered in methods to treat HAE, such compositions will contain an amount of active ingredient effective to achieve the desired result (e.g. inhibiting PKa and/or decreasing the amount of bradykinin in a subject).

The dosage and frequency (single or multiple doses) of compound administered can vary depending upon a variety of factors, including route of administration; size, age, sex, health, body weight, body mass index, and diet of the recipient; nature and extent of symptoms of the disease being treated (e.g., the disease responsive to PKa inhibition); presence of other diseases or other health-related problems; kind of concurrent treatment; and complications from any disease or treatment regimen. Other therapeutic regimens or agents can be used in conjunction with the methods and compounds of the invention.

For any provided compound or test agent, the therapeutically effective amount can be initially determined from cell culture assays. Target concentrations will be those concentrations of active compound(s) that are capable of decreasing PKa enzymatic activity as measured, for example, using the methods described.

Therapeutically effective amounts for use in humans may be determined from animal models. For example, a dose for humans can be formulated to achieve a concentration that has been found to be effective in animals. The dosage in humans can be adjusted by monitoring PKa inhibition and adjusting the dosage upwards or downwards, as described above.

Dosages may be varied depending upon the requirements of the patient and the compound being employed. The dose administered to a patient, in the context of the present invention, should be sufficient to effect a beneficial therapeutic response in the patient over time. The size of the dose also will be determined by the existence, nature, and extent of any adverse side effects.

In one aspect, compounds provided herein display one or more improved pharmacokinetic (PK) properties (e.g., Cₘₐₓ, tₘₐₓ, Cₘᵢₙ, t_{1/2}, AUC, CL, bioavailability, etc.) when compared to a reference compound. In some embodiments, a reference compound is a PKa inhibitor known in the art. In some embodiments, a reference compound is a PKa inhibitor selected from those disclosed in PCT Publication Number WO 2019/178129.

In some embodiments a compound of the disclosure or a pharmaceutical composition comprising the same is provided as a unit dose.

### F. Methods of Treatment

The present disclosure provides compounds and pharmaceutical compositions comprising the same for use in medicine i.e. for use in treatment. The present disclosure further provides the use of any compounds described herein for inhibiting the activity of PKa, which would be beneficial to treatment of PKa-mediated diseases and conditions. Exemplary PKa-mediated disorders include edema, which refers to swelling in the whole body of a subject or a part thereof due to inflammation or injury when small blood vessels become leaky and releases fluid into nearby tissues. In some examples, the edema is HAE. In other examples, the edema occurs in eyes, e.g., diabetic macular edema (DIE). The present disclosure provides methods of inhibiting the activity of PKa. In certain embodiments, the application provides a method of inhibiting the activity of PKa in vitro via contacting any of the compounds described herein with PKa molecules in a sample, such as a biological sample. In certain embodiments, the application provides a method of inhibiting the activity of PKa in vivo via delivering an effective amount of any of the compounds described herein to a subject in need of the treatment through a suitable route.

In certain embodiments, the methods comprise administering to a subject in need thereof (e.g., a subject such as a human patient with edema) any of the compounds described herein or a pharmaceutically acceptable salt thereof. In certain embodiments, the methods comprise administering a compound of Formulae (I)-(VIII-c) or compounds I-57, I-59 through I-61, or I-153, or a pharmaceutically acceptable salt or composition thereof, to a subject in need thereof. In some embodiments, the method comprises administering a pharmaceutical composition comprising a compound of Formulae (I)-(VIII-c) or compounds I-57, I-59 through I-61, or I-153, or a pharmaceutically acceptable salt to a subject in need thereof.

In certain embodiments, the subject to be treated by any of the methods described herein is a human patient having, suspected of having, or at risk for edema, for example, HAE or diabetic macular edema (DIE). A subject having an edema can be identified by routine medical examination, e.g., laboratory tests. A subject suspected of having an edema might show one or more symptoms of the disease/disorder. A subject at risk for edema can be a subject having one or more of the risk factors associated with the disease, for example, deficiency in C1-INH as for HAE.

In certain embodiments, provided herein are methods of alleviating one or more symptoms of HAE in a human patient who is suffering from an HAE attack. Such a patient can be identified by routine medical procedures. An effective amount of one or more of the provided compounds can be given to the human patient via a suitable route, for example, those described herein. The compounds described herein may be used alone, or may be used in combination with other anti-HAE agents, for example, a C1 esterase inhibitor (e.g., Cinryze® or Berinert®), a PKa inhibitor (e.g., ecallantide or lanadelumab) or a bradykinin B2 receptor antagonist (e.g., Firazyr®).

In other embodiments, provided herein are methods or reducing the risk of HAE attack in a human HAE patient who is in quiescent stage. Such a patient can be identified based on various factors, including history of HAE attack. An effective amount of one or more of the compounds can be given to the human patient via a suitable route, for example, those described herein. The compounds described herein may be used alone, or may be used in combination with other anti-HAE agents, for example, a C₁ esterase inhibitor (e.g., Cinryze® or Berinert®), a PKa inhibitor (e.g., ecallantide or lanadelumab) or a bradykinin B2 receptor antagonist (e.g., Firazyr®).

In some embodiments, provided herein is prophylactic treatment of HAE in human patients having risk to HAE attacks with one or more of the compounds described herein. In some embodiments, patients suitable for prophylactic treatment of HAE are human subjects suffering from HAE (e.g., having history of HAE attacks). In some embodiments, patients suitable for such prophylactic treatment are human subjects where a physician determines a history of HAE attacks warrants a prophylactic approach (e.g., human subjects experiencing more than a particular average number of attacks over a time period, including by way of nonlimiting example, one, two, or more attacks per month). Alternatively, patients suitable for the prophylactic treatment may be human subjects having no HAE attack history but bearing one or more risk factors for HAE (e.g., family history, genetic defects in C1-INH gene, etc.) Such prophylactic treatment may involve the compounds described herein as the sole active agent, or involve additional anti-HAE agents, such as those described herein.

In certain embodiments, provided herein are methods for preventing or reducing edema in an eye of a subject (e.g., a human patient). In some examples, the human patient is a diabetic having, suspected of having, or at risk for diabetic macular edema (DME). DME is the proliferative form of diabetic retinopathy characterized by swelling of the retinal layers, neovascularization, vascular leak, and retinal thickening in diabetes mellitus due to leaking of fluid from blood vessels within the macula. To practice this method, an effective amount of one or more of the compounds described herein, or pharmaceutically acceptable salts thereof, may be delivered into the eye of the subject where treatment is needed. For example, the compound may be delivered topically, by intraocular injection, or intravitreal injection. A subject may be treated with the compound as described herein, either as the sole active agent, or in combination with another treatment for DME. Non-limiting examples of treatment for DME include laser photocoagulation, steroids, VEGF pathway targeting agents (e.g., Lucentis® (ranibizumab) or Eylea® (aflibercept)), and/or anti-PDGF agents.

In certain embodiments, the methods disclosed herein comprise administering to the subject an effective amount of a compound of Formulae (I)-(VIII-c) or compounds I-57, I-59 through I-61, or I-153, or a pharmaceutically acceptable salt or composition thereof. In some embodiments, the effective amount is a therapeutically effective amount. In some embodiments, the effective amount is a prophylactically effective amount.

In certain embodiments, the subject being treated is an animal. The animal may be of either sex and may be at any stage of development. In certain embodiments, the subject is a mammal. In certain embodiments, the subject being treated is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent (e.g., mouse, rat), dog, pig, or non-human primate. In certain embodiments, the animal is a genetically engineered animal. In certain embodiments, the animal is a transgenic animal.

Certain methods described herein may comprise administering one or more additional pharmaceutical agent(s) in combination with the compounds described herein. The additional pharmaceutical agent(s) may be administered at the same time as the compound of Formulae (I)-(VIII-c) or compounds I-57, I-59 through I-61, or I-153, or at different times than the compound of Formulae (I)-(VIII-c) or compounds I-57, I-59 through I-61, or I-153. For example, the compound of Formulae (I)-(VIII-c) or compounds I-57, I-59 through I-61, or I-153 and any additional pharmaceutical agent(s) may be on the same dosing schedule or different dosing schedules. All or some doses of the compound of Formulae (I)-(VIII-c) or compounds I-57, I-59 through I-61, or I-153 may be administered before all or some doses of an additional pharmaceutical agent, after all or some does an additional pharmaceutical agent, within a dosing schedule of an additional pharmaceutical agent, or a combination thereof. The timing of administration of the compound of Formulae (I)-(VIII-c) or compounds I-57, I-59 through I-61, or I-153 and additional pharmaceutical agents may be different for different additional pharmaceutical agents.

Also provided is use of a compound of the present disclosure for the manufacture of a medicament for a condition/disease disclosed herein.

In certain embodiments, the additional pharmaceutical agent comprises an agent useful in the treatment of an edema, such as HAE or DME. Examples of such agents are provided herein.

In the context of this specification “comprising” is to be interpreted as “including”. Embodiments of the invention comprising certain features/elements are also intended to extend to alternative embodiments “consisting” or “consisting essentially” of the relevant elements/features. Where technically appropriate, embodiments of the invention may be combined.

Technical references such as patents and applications are incorporated herein by reference.

Any embodiments specifically and explicitly recited herein may form the basis of a disclaimer either alone or in combination with one or more further embodiments.

The background section of this specification contains relevant technical information and may be used as basis for amendment. Subject headings herein are employed to divide the document into sections and are not intended to be used to construe the meaning of the disclosure provided herein.

The present specification claims priority from U.S. Provisional Application No. 63/162,477 (filed Mar. 17, 2021) incorporated herein by reference. This application may be used as basis for corrections to the present specification, especially in respect of chemical structures disclosed therein.

### IV. EXAMPLES

In certain embodiments, the Examples describe compounds comprising one or more stereocenters, where a particular stereocenter is designated “S*” or “R*.” In both cases, the depiction of the “*” generally indicates that the exact configuration is unknown (e.g., for a compound with a single stereocenter, the depiction R*- or S*- indicates that either the R- or S-isomer was isolated, but the configuration at the stereocenter of the particular isomer isolated was not determined).

It will be appreciated that compounds described within the Examples may comprise more than one stereocenter. As described above, single stereochemical isomers as well as enantiomeric, diastereomeric, and geometric (or conformational) mixtures of the present compounds are within the scope of the invention. Within a particular compound name, where more than one “S*” or “R*” appear within a single pair of parentheses (e.g., “(1S*,2S*)”), it is understood that the S* and/or R* configurations are relative to each other. For example, a compound denoted “(1S*,2S*)—” or “(1R*,2R*)—” would be understood to refer specifically to either the “(1S,2S)—” or “(1R,2R)—” isomer, but not the “(1S,2R)—” or “(1R,2S)—” isomers. Furthermore, a compound denoted “rac-(1S*,2S*)—” or “rac-(1R*,2R*)—” would be understood to include a racemic mixture of the “(1S,2S)—” and “(1R,2R)—” isomers. Similarly, a compound denoted “(1S*,2R*)—” or “(1R*,2S*)—” would be understood to refer specifically to either the “(1R,2S)—” or “(1S,2R)—” isomer, but not the “(1S,2S)—” or “(1R,2R)—” isomers. In addition, a compound denoted “rac-(1R*,2S*)—” or “rac-(1S*,2R*)—” would be understood to include a racemic mixture of the “(1R,2S)—” and “(1S,2R)—” isomers.

In certain embodiments, the Examples include schemes that depict compounds with one or more stereocenters. In some embodiments, the symbol “&” followed by a number appears adjacent to a stereocenter. In such cases, it is understood to include a mixture (e.g., a racemic mixture) of both stereoisomers (e.g., R- and S-) at that position.

In some embodiments, the term “or” followed by a number appears adjacent to a stereocenter. In such cases, it is understood to denote either an “R-” or “S-” isomer, but the particular isomer was not determined.

### Synthesis of Intermediates

### Synthesis of 2-(7-chloroimidazo[1,5-a]pyridin-1-yl)acetic acid

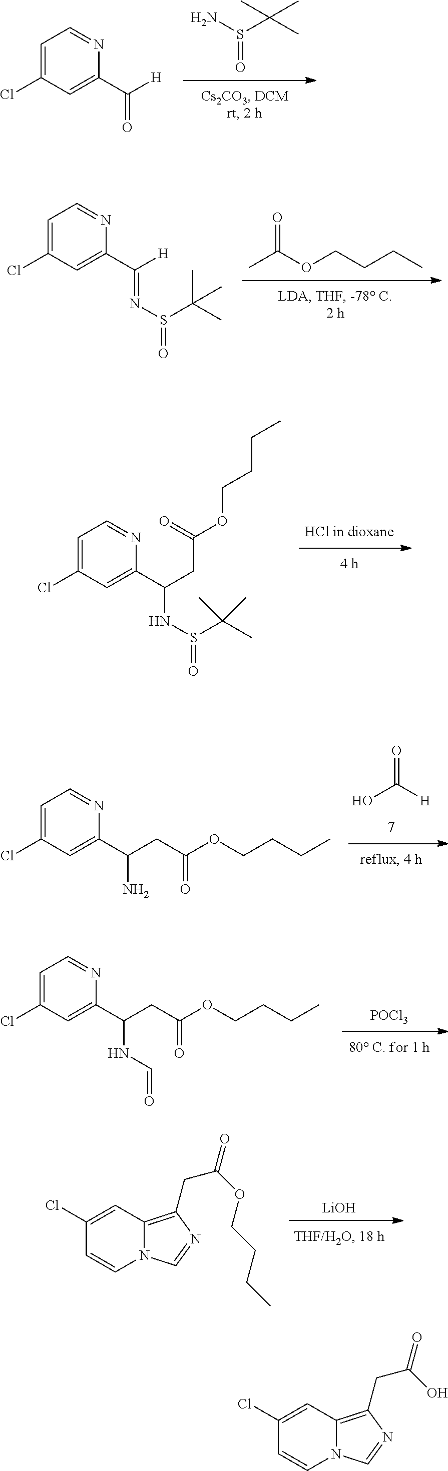

Synthesis of N-((4-chloropyridin-2-yl)methylene)-2-methylpropane-2-sulfinamide. To a stirred suspension of (S)-2-methylpropane-2-sulfinamide (5 g, 41.3 mmol) and CS₂CO₃ (20.16 g, 61.9 mmol) in DCM (100 mL) was added a solution of 4-chloropicolinaldehyde (5.84 g, 41.3 mmol) in DCM (20 mL) dropwise over a period of 10 min at room temperature. The solution was stirred for 2 h. Then the reaction mixture was diluted with water (100 mL) and extracted with DCM (3×100 mL). The combined organic layer was dried over MgSO₄ and concentrated to give the title compound (10.1 g) as a brown oil which was used in the next step without purification. ESI-MS (M+H)+: 246.9.

Synthesis of 3-((tert-butylsulfinyl)amino)-3-(4-chloropyridin-2-yl)propanoate. To a stirred solution of LDA (2 M in THF, 43 mL, 86 mmol) in anhydrous THF (100 mL) was added dropwise a solution of butyl acetate (9.4 g, 81 mmol) in THF (20 mL) at −78° C. under a nitrogen atmosphere. After stirring for 30 min, a solution of N-((4-chloropyridin-2-yl)methylene)-2-methylpropane-2-sulfinamide (10.0 g, 40.98 mmol) in THF (30 mL) was added at this temperature. After stirring for another 2 hours at −78° C. the reaction mixture was quenched by saturated ammonium chloride (100 mL) and warmed to room temperature. The organic layer was separated, and the aqueous layer was extracted with EtOAc (3×100 mL). The combined organic layers were dried over Na₂SO₄, concentrated and purified by column chromatography (PE:EtOAc=50:50) to give the title compound (9.0 g, 61%) as a white solid. ESI-MS (M+H)⁺: 361.

Synthesis of butyl 3-amino-3-(4-chloropyridin-2-yl)propanoate. To a solution of 3-((tert-butylsulfinyl)amino)-3-(4-chloropyridin-2-yl)propanoate (3.5 g, 9.7 mmol) in dioxane (30 mL) was added a solution of HCl (4 M in dioxane, 10 mL, 40 mmol). The mixture was stirred at room temperature for 4 h and then concentrated to give the title compound as a crude oil (4.3 g) which was used in the next step without purification. ESI-MS (M+H)⁺: 257.

Synthesis of butyl 3-(4-chloropyridin-2-yl)-3-formamidopropanoate. A solution of butyl 3-amino-3-(4-chloropyridin-2-yl)propanoate (2.6 g, 10.2 mmol) in formic acid (10 mL) was stirred at reflux for 4 h. The reaction mixture was then concentrated to give the title compound as a dark oil (3.0 g) which was used into next step without purification. ESI-MS (M+H)⁺: 285.

Synthesis of butyl 2-(7-chloroimidazo[1,5-a]pyridin-1-yl)acetate. A solution of butyl 3-(4-chloropyridin-2-yl)-3-formamidopropanoate (500 mg, 1.76 mmol) in POCl₃ (5 mL) was stirred at 80° C. for 1 h. The mixture was evaporated, the residue dissolved in EtOAc (100 mL) and washed with saturated NaHCO₃ solution (50 mL). The organic layer was separated, and the aqueous layer was extracted with EtOAc (3×50 mL). The combined organic layers were concentrated and purified by silica gel chromatography (PE:EtOAc=30:70) to give the title compound (290 mg, yield: 62%). ESI-MS (M+H)⁺: 267.

Synthesis of 2-(7-chloroimidazo[1,5-a]pyridin-1-yl)acetic acid. To a stirred solution of butyl 2-(7-chloroimidazo[1,5-a]pyridin-1-yl)acetate (310 mg, 1.16 mmol) in THF/H₂O (3/1, 4 mL) was added LiOH (139 mg, 5.8 mmol). After the mixture was stirred at room temperature for 16 h, the pH was adjusted to 6 by the addition of aqueous HCl (1 M) and extracted with DCM/MeOH (10/1, 5×50 mL). The combined organic layer was dried and concentrated to give the title compound (200 mg), which was used without further purification. ESI-MS (M+H)⁺: 211.

### Synthesis of methyl 2-(7-chloroimidazo[1,5-a]pyridin-1-yl)acetate

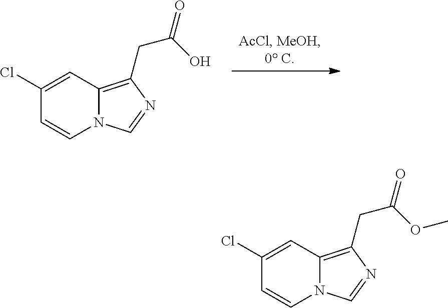

Acetyl chloride (0.42 mL, 5.93 mmol) was added to a cooled solution of 2-(7-chloroimidazo[1,5-a]pyridin-1-yl)acetic acid (25 mg, 1.19 mmol) at 0° C. in MeOH (5 mL). The mixture was allowed to warm to room temperature and stirred for 18 h. The residue was treated with NaHCO₃ (aq. sat., 25 mL) and water (25 mL) and extracted with 10% MeOH in DCM (3×50 mL). The combined organic phases were dried (MgSO₄), filtered and concentrated in vacuo to give the title compound (310 mg, quant.) which was used without further purification. ¹H NMR (400 MHz, DMSO) δ8.37 (s, 1H), 8.36 (d, J=0.8 Hz, 1H), 7.82-7.80 (m, 1H), 6.71 (dd, J=2.1, 7.5 Hz, 1H), 3.96 (s, 2H), 3.66 (s, 3H).

### Synthesis of 2-(7-chloroimidazo[1,5-a]pyridin-1-yl)propanoic acid

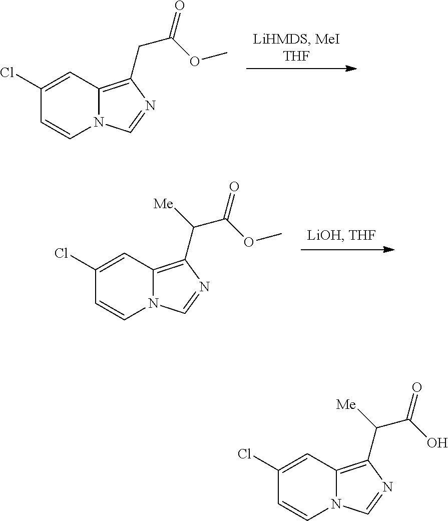

Synthesis of methyl 2-(7-chloroimidazo[1,5-a]pyridin-1-yl)propanoate. To a solution of methyl 2-(7-chloroimidazo[1,5-a]pyridin-1-yl)acetate (2.0 g, 8.9 mmol) in of tetrahydrofuran (30 mL) was added lithium bis(trimethylsilyl)amide (1M in THF, 9.8 mL, 9.8 mmol) in portions. The reaction mixture was stirred at room temperature for 2 h. Iodomethane (1.39 g, 9.8 mmol) was added and the mixture was stirred at ambient temperature for 2 h. The reaction mixture was diluted with water and extracted with ethyl acetate (3×50 mL). The combined organic layers were washed with brine, dried over anhydrous sodium sulphate, filtered, and concentrated. The residue was purified by silica gel column chromatography (ethyl acetate in petroleum ether=0˜100%) to afford the title compound (0.9 g, yield: 38.5%) as a yellow oil.

Synthesis of 2-(7-chloroimidazo[1,5-a]pyridin-1-yl)propanoic acid. To a solution of methyl 2-(7-chloroimidazo[1,5-a]pyridin-1-yl)propanoate (0.9 g, 3.77 mmol) in tetrahydrofuran (15 mL), methanol (15 mL) and water (15 mL) was added lithium hydroxide hydrate (186 mg, 7.54 mmol). The resulting reaction mixture was stirred at room temperature for 4 h. After evaporation, the mixture was acidified with 1 N HCl to pH 4. The precipitate was filtered and dried under vacuum to give the title compound (560 mg, yield: 66%) as a yellow solid. ¹HNMR (400 Hz, DMSO-d₆) δ12.30 (br, 1H), 8.32 (s, 1H), 8.31 (d, J=6.4 Hz, 1H), 7.72 (t, J=0.8 Hz, 1H), 6.65 (dd, J=7.2, 2.0 Hz, 1H), 4.10 (q, J=7.2 Hz, 1H), 1.45 (d, J=7.2 Hz, 3H).

### Synthesis of 2-(7-chloroimidazo[1,5-a]pyridin-1-yl)-2-methylpropanoic acid

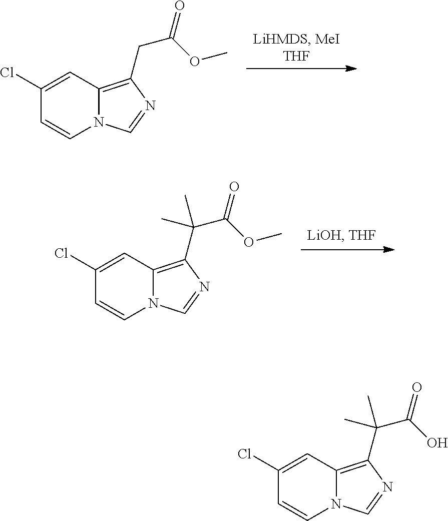

Synthesis of methyl 2-(7-chloroimidazo[1,5-a]pyridin-1-yl)-2-methylpropanoate. To a solution of methyl 2-(7-chloroimidazo[1,5-a]pyridin-1-yl)acetate (2.0 g, 8.9 mmol) in tetrahydrofuran (30 mL) was added lithium bis(trimethylsilyl)amide (1M in THF, 22 mL, 22 mmol) in portions. The reaction mixture was stirred at room temperature for 2 h. Iodomethane (3.16 g, 22.3 mmol) was added and the mixture was stirred at ambient temperature for 2 h. The reaction mixture was diluted with water and extracted with ethyl acetate (3×50 mL). The combined organic layers were washed with brine, dried over anhydrous sodium sulphate, filtered, and concentrated. The residue was purified by silica gel column chromatography (ethyl acetate in petroleum ether=0 to 100%) to afford the title compound (1.3 g, crude) as a yellow oil. ESI-MS (M+H)⁺: 253.1

Synthesis of 2-(7-chloroimidazo[1,5-a]pyridin-1-yl)-2-methylpropanoic acid. To a solution of methyl 2-(7-chloroimidazo[1,5-a]pyridin-1-yl)-2-methylpropanoate (1.25 g crude, 4.95 mmol) in tetrahydrofuran (15 mL), methanol (15 mL) and water (15 mL) was added lithium hydroxide hydrate (415 mg, 9.89 mmol). The resulting reaction mixture was stirred at room temperature for 4 hrs. After evaporation, the mixture was acidified with 1 N HCl to pH 4. The precipitate was filtered and dried under vacuum to give the title compound (300 mg) as a yellow solid. ¹HNMR (400 Hz, DMSO-d₆) δ 8.22 (m, 2H), 7.39 (i, 1H), 6.55 (m, 1H), 1.46 (s, 6H). Carboxylic acid proton not observed.

### Synthesis of 2-(7-chloroimidazo[1,5-a]pyridin-1-yl)acetamide

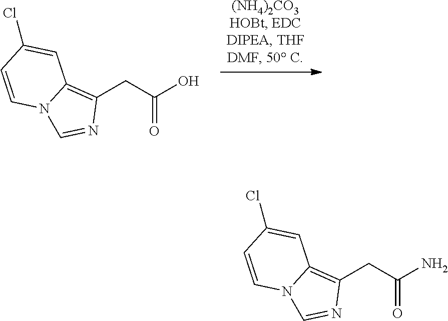 [SMILES: O=C(O)Cc1ncn2ccc(Cl)cc12 ; "(NH4)2CO3HOBt, EDCDIPEA, THFDMF, 50° C." ; NC(=O)Cc1ncn2ccc(Cl)cc12]

A mixture of 2-(7-chloroimidazo[1,5-a]pyridin-1-yl)acetic acid (2.0 g, 9.5 mmol), EDC (1.8 g, 11 mmol), HOBt (1.5 g, 11 mmol), DIPEA (9.9 mL, 57 mmol) and (NH₄)₂C₃ (4.6 g, 47 mmol) in THF (20 mL) and DMF (10 mL) was stirred at 60° C. for 18 h. The mixture was cooled and diluted with EtOAc. The organic layer was washed with NaHCO₃ (sat. aq.) and brine (sat. aq.). The combined aqueous layers were further extracted with copious DCM. The combined organic layers were dried (MgSO₄), filtered and concentrated in vacuo to give the title compound (0.80 g, 400%). ESI-MS (M+H)⁺: 210, ¹H NMR (400 MHz, DMSO) δ8.37-8.32 (m, 2H), 7.78 (s, 1H), 7.40 (s, 1H), 6.97 (s, 1H), 6.70-6.65 (m, 1H), 3.66 (s, 2H).

The intermediates below were prepared in the same manner as described above for 2-(7-chloroimidazo[1,5-a]pyridin-1-yl)acetamide with the residue being purified by column chromatography on silica gel, eluting with a gradient of 0-100% EtOAc in DCM to give the titled compound.

| Intermediate | Starting acid | Analytical data |
|---|---|---|
| 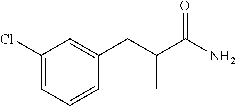 3-(3-chlorophenyl)-2- methylpropanamide | 3-(3-chlorophenyl)-2- methylpropanoic acid | ESI-MS (M + H)⁺: 198.1 ¹H NMR (400 MHz, CDCl₃) δ 7.24-7.18 (m, 3H), 7.09-7.06 (m, 1H), 5.66 (s, 1H), 5.37 (s, 1H), 2.97 (dd, J = 7.8, 13.4 Hz, 1H), 2.64 (q, J = 6.7 Hz, 1H), 2.58-2.46 (m, 1H), 1.19 (d, J = 6.8 Hz, 3H). |
| 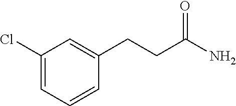 [SMILES: NC(=O)CCc1cccc(Cl)c1] 3-(3-chlorophenyl)propanamide | 3-(3- chlorophenyl)propanoic acid | ESI-MS (M + H)⁺: 184.1 ¹H NMR (400 MHz, CD₃OD) δ 7.30- 7.17 (m, 4H), 2.93 (t, J = 7.7 Hz, 2H), 2.53 (t, J = 7.7 Hz, 2H). 2 exchangeable protons not observed |
| 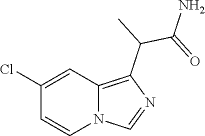 2-(7-chloroimidazo[1,5- a]pyridin-1-yl)propanamide | 2-(7-chloroimidazo[1,5- a]pyridin-1-yl)propanoic acid | ¹H NMR (400 MHz, DMSO) δ 8.31 (d, J = 6.7 Hz, 2H), 7.79-7.77 (m, 1H), 7.30 (s, 1H), 6.87 (s, 1H), 6.64 (dd, J = 2.1, 7.5 Hz, 1H), 3.90 (q, J = 7.2 Hz, 1H), 1.42 (d, J = 7.2 Hz, 3H). |
| 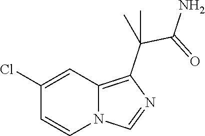 [SMILES: CC(C)(C(N)=O)c1ncn2ccc(Cl)cc12] 2-(7-chloroimidazo[1,5- a]pyridin-1-yl)-2-methylpropanamide | 2-(7-chloroimidazo[1,5- a]pyridin-1-yl)-2- methylpropanoic acid | ¹H NMR (400 MHz, DMSO) δ 8.39- 8.35 (m, 2H), 7.55 (s, 1H), 6.99 (s, 2H), 6.68 (dd, J = 1.9, 7.5 Hz, 1H), 1.59-1.56 (m, 6H). |

### Synthesis of perfluorophenyl 2-(7-chloroimidazo[1,5-a]pyridin-1-yl)acetate

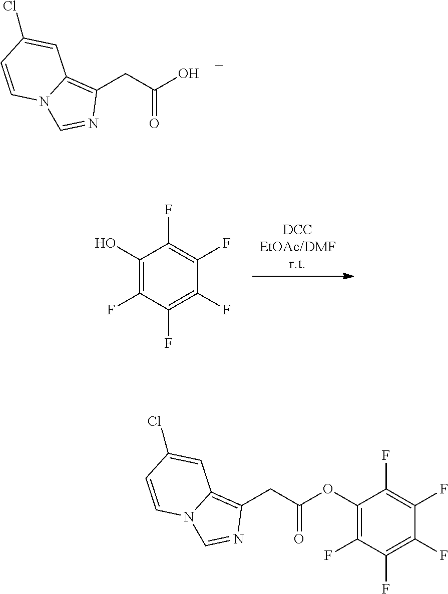 [SMILES: O=C(O)Cc1ncn2ccc(Cl)cc12 ; Oc1c(F)c(F)c(F)c(F)c1F ; "DCCEtOAc/DMFr.t." ; O=C(Cc1ncn2ccc(Cl)cc12)Oc1c(F)c(F)c(F)c(F)c1F]

DCC (0.54 g, 2.6 mmol) was added to a stirred suspension of 2-(7-chloroimidazo[1,5-a]pyridin-1-yl)acetic acid (0.50 g, 2.4 mmol) in EtOAc (25 mL). After 5 min a solution of 2,3,4,5,6-pentafluorophenol (0.48 g, 2.6 mmol) in DMF (2.5 mL) was added and the mixture was stirred at room temperature for 18 h. The mixture was filtered and concentrated in vacuo. The residue was dissolved in EtOAc and a small quantity of Et₂O was added. The mixture was filtered and concentrated in vacuo. The residue was purified by column chromatography on silica gel, eluting with 20-80% EtOAc in isohexane. The residue was triturated with isohexane and dried to give the title compound (0.45 g, 51%). ¹H NMR (400 MHz, DMSO) δ8.45 (s, 1H), 8.41 (d, J=7.3 Hz, 1H), 7.97-7.94 (m, 1H), 6.78-6.73 (m, 1H), 4.49-4.47 (m, 2H).

### Synthesis of methyl 2-(7-chloroimidazo[1,5-a]pyridin-1-yl)-2-hydroxyacetate

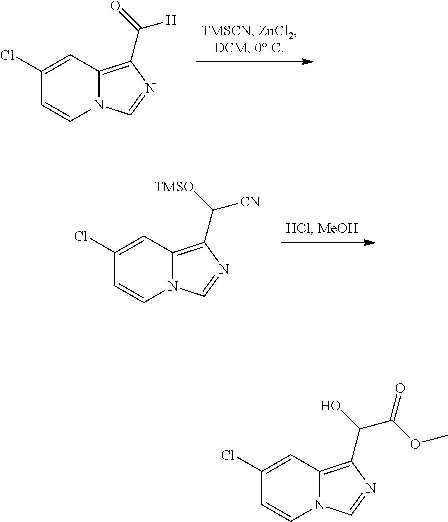

Synthesis of 2-(7-chloroimidazo[1,5-a]pyridin-1-yl)-2-((trimethylsilyl)oxy)acetonitrile. To a solution of 7-chloroimidazo[1,5-a]pyridine-1-carbaldehyde (4.5 g, 25.0 mmol, WO2019178129) in DCM (50 mL) was added trimethylsilyl cyanide (5 mL, 50.0 mmol) and zinc iodide (1.0 g, 2.5 mmol), then the mixture was stirred at room temperature overnight. The reaction was quenched with sodium bicarbonate and extracted with DCM (3×200 mL). The combined organic layers were washed with brine, dried over magnesium sulphate, filtered, and concentrated to afford the title compound (7.0 g, crude) as a brown solid which was used without further purification. ESI-MS (M+H)⁺: 280.1.

Synthesis of methyl 2-(7-chloroimidazo[1,5-a]pyridin-1-yl)-2-hydroxyacetate. A mixture of 2-(7-chloroimidazo[1,5-a]pyridin-1-yl)-2-((trimethylsilyl)oxy)acetonitrile (6.0 g 21.4 mmol) and hydrogen chloride (in methanol, 15 mL) was stirred at room temperature for 24 h. The reaction mixture was quenched with water, adjusted pH to 8 with sodium bicarbonate and extracted with dichloromethane (3×80 mL). The combined organic layers were washed with brine, dried over with anhydrous magnesium sulphate, filtered, and concentrated to get the crude product, which was purified by silica gel column chromatography using ethyl acetate to afford the title compound (1.0 g, 60%) as a yellow solid. ESI-MS (M+H)⁺: 240.9, ¹H NMR (400 Hz, DMSO) δ8.36 (d, J=7.2 Hz, 1H), 8.32 (s, 1H), 7.81 (s, 1H), 6.72 (dd, J=7.2, 2.0 Hz, 1H), 5.97 (d, J=5.6 Hz, 1H), 5.50 (d, J=5.6 Hz, 1H), 3.62 (s, 3H).

### Synthesis of 2-(7-chloroimidazo[1,5-a]pyridin-1-yl)-2-methoxyacetamide

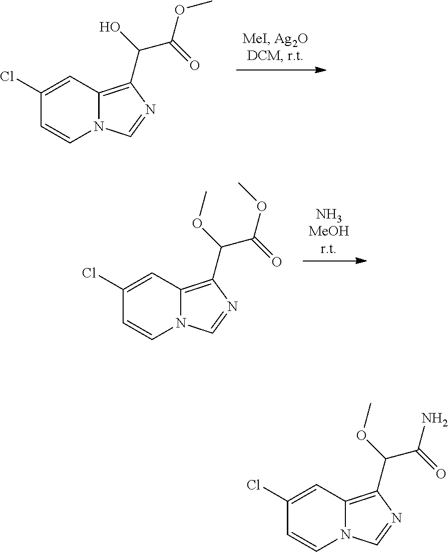 [SMILES: COC(=O)C(O)c1ncn2ccc(Cl)cc12 ; "MeI, Ag2ODCM, r.t." ; COC(=O)C(OC)c1ncn2ccc(Cl)cc12 ; "NH3MeOHr.t." ; COC(C(N)=O)c1ncn2ccc(Cl)cc12]

Synthesis of methyl 2-(7-chloroimidazo[1,5-a]pyridin-1-yl)-2-methoxyacetate. MeI (0.019 mL, 0.30 mmol) was added to a stirred mixture of methyl 2-(7-chloroimidazo[1,5-a]pyridin-1-yl)-2-hydroxyacetate (0.048 g, 0.20 mmol) and Ag₂O (0.070 g, 0.30 mmol) in DCM (5.0 mL). The mixture was stirred at room temperature for 18 h then further MeI (0.019 mL, 0.30 mmol) was added. The mixture was stirred at room temperature for 2 h then further MeI (0.019 mL, 0.30 mmol) was added. The mixture was stirred at room temperature for 18 h then further MeI (0.13 mL, 2.0 mmol) was added in 6 portions over 8 h. The mixture was filtered through Celite® and concentrated in vacuo. The residue was purified by column chromatography on silica gel, eluting with 1-5% MeOH in DCM to give the title compound (0.032 g, 62%) as a yellow gum. ¹H NMR (400 MHz, CDCl₃) δ8.03 (s, 1H), 7.84 (dd, J=1.0, 7.4 Hz, 1H), 7.69-7.68 (m, 1H), 6.57 (dd, J=2.0, 7.5 Hz, 1H), 5.20 (s, 1H), 3.77 (s, 3H), 3.44 (s, 3H).

Synthesis of 2-(7-chloroimidazo[1,5-a]pyridin-1-yl)-2-methoxyacetamide. Ammonia (7N in MeOH, 0.43 mL, 3.0 mmol) was added to a solution of methyl 2-(7-chloroimidazo[1,5-a]pyridin-1-yl)-2-methoxyacetate (76 mg, 0.30 mmol) in MeOH (2 mL) and stirred at room temperature for 48 h. The mixture was concentrated in vacuo and the residue was triturated in diethyl ether to give the title compound (22 mg, 31%) as a pale brown solid. ¹H NMR (400 MHz, DMSO) δ8.44-8.39 (m, 2H), 7.90-7.86 (m, 1H), 7.59 (s, 1H), 7.37 (s, 1H), 6.79-6.74 (m, 1H), 4.98 (s, 1H), 3.29 (s, 3H).

### Synthesis of 2-(7-chloroimidazo[1,5-a]pyridin-1-yl)-2-hydroxyacetamide

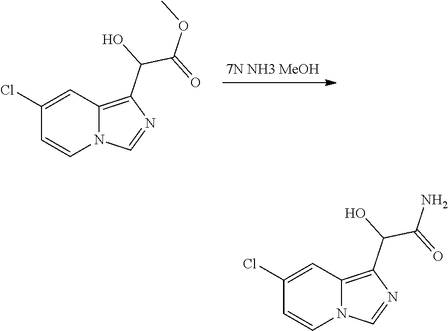 [SMILES: COC(=O)C(O)c1ncn2ccc(Cl)cc12 ; NC(=O)C(O)c1ncn2ccc(Cl)cc12]

Ammonia (7 N in methanol, 1.4 mL) was added to a methyl 2-(7-chloroimidazo[1,5-a]pyridin-1-yl)-2-hydroxyacetate (241 mg, 1.0 mmol) in MeOH (1 mL) and the resulting mixture stirred at room temperature for 18 h. The precipitate formed was filtered, washed with MeOH and dried at 48° C. in vacuo to give the title compound (162 mg, 72%) as an off-white solid. ESI-MS (M+H)⁺: 226/228.

### Synthesis of 2-(7-bromo-8-fluoroimidazo[1,5-a]pyridin-1-yl)acetic acid

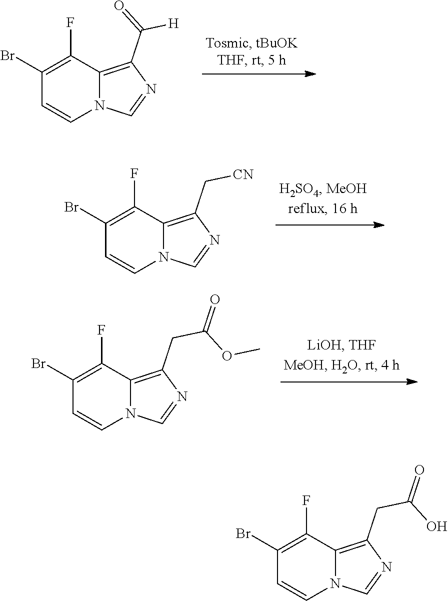

Synthesis of 2-(7-bromo-8-fluoroimidazo[1,5-a]pyridin-1-yl)acetonitrile. To a solution of 7-bromo-8-fluoroimidazo[1,5-a]pyridine-1-carbaldehyde (2.43 g, 10 mmol, see WO2019178129) and tosylmethyl isocyanide (2.34 g, 12 mmol) in tetrahydrofuran (50 mL) was added potassium tert-butanolate (2.24 g, 20 mmol) in portions. The reaction mixture was stirred at room temperature for 2 h. Methanol (50 mL) was added and the mixture was stirred at reflux for 2 h. The reaction mixture was concentrated, diluted with water and extracted with ethyl acetate (3×50 mL). The combined organic layers were washed with brine, dried over anhydrous sodium sulphate, filtered, and concentrated. The residue was purified by silica gel column chromatography (ethyl acetate in petroleum ether=0-100%) to afford the title compound (1.1 g, 43%) as a yellow solid. ¹HNMR (400 Hz, CDCl₃) δ8.12 (d, J=2.4 Hz, 1H), 7.64 (d, J=7.2 Hz, 1H), 6.67 (t, J=6.8 Hz, 1H), 4.09 (s, 2H).

Synthesis of methyl 2-(7-bromo-8-fluoroimidazo[1,5-a]pyridin-1-yl)acetate. To a solution of 2-(7-bromo-8-fluoroimidazo[1,5-a]pyridin-1-yl)acetonitrile (1.1 g, 4.35 mmol) in methanol (50 mL) was added conc H₂SO₄ (10 mL) dropwise at 0° C. The reaction mixture was stirred at reflux overnight. The solvent was removed under vacuo and the residue was basified with saturated sodium bicarbonate solution, then extracted with ethyl acetate (3×50 mL). The combined organic layers were washed with brine, dried over sodium sulphate, filtrated, evaporated and purified by silica gel column chromatography (petrol ether:ethyl acetate=1:10) to give the title compound (1.0 g, yield: 80%) as a yellow solid. ¹HNMR (400 Hz, DMSO) δ8.45 (d, J=2.4 Hz, 1H), 8.14 (d, J=7.2 Hz, 1H), 6.83 (t, J=6.8 Hz, 1H), 3.93 (s, 2H), 3.62 (s, 3H).

Synthesis of 2-(7-bromo-8-fluoroimidazo[1,5-a]pyridin-1-yl)acetic acid. To a solution of 2-(7-bromo-8-fluoroimidazo[1,5-a]pyridine-1-yl)acetate (1.0 g, 3.48 mmol) in tetrahydrofuran (10 mL), methanol (10 mL) and water (5 mL) was added lithium hydroxide hydrate (439 mg, 10.45 mmol). The resulting reaction mixture was stirred at room temperature for 4 h. After evaporation, the mixture was acidified with 1 N HCl to pH 4. The precipitate was filtered and dried under vacuum to give the title compound (900 mg, yield: 95%) as a white solid. ¹HNMR (400 Hz, DMSO) δ12.43 (br, 1H), 8.44 (d, J=2.4 Hz, 1H), 8.14 (d, J=7.2 Hz, 1H), 6.83-6.80 (m, 1H), 3.82 (s, 2H).

### Synthesis of 2-(5-chloro-2-cyanophenyl)acetamide

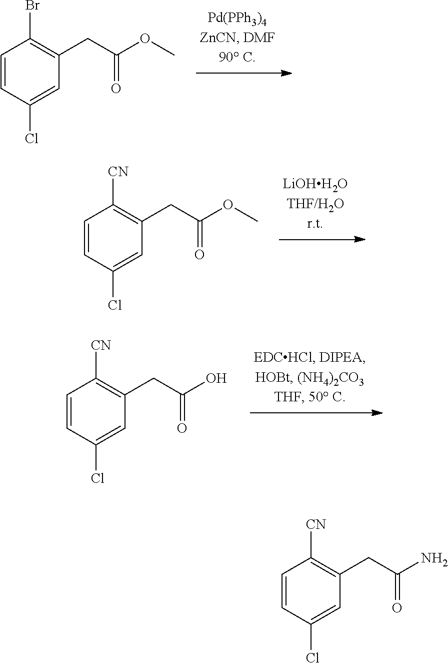

Synthesis of methyl 2-(5-chloro-2-cyanophenyl)acetate. Pd(PPh₃)₄ (320 mg, 0.277 mmol) was added to a nitrogen purged mixture of methyl 2-(2-bromo-5-chlorophenyl)acetate (3.65 g, 13.9 mmol), zinc cyanide (0.85 g, 7.2 mmol) in DMF (28 mL) and heated to 90° C. for 18 h. The mixture was diluted with water (100 mL), extracted with EtOAc (2×100 mL), the combined organic phase was washed with LiCl (4%, aq., 40 mL), dried (MgSO₄) and concentrated in vacuo. Purification by column chromatography on silica gel, eluting with a gradient of 0-60% Et₂O in cyclohexane gave the title compound (2.11 g, 72%). ¹H NMR (400 MHz, CDCl₃) δ7.62-7.59 (m, 1H), 7.44 (d, J=1.8 Hz, 1H), 7.39 (q, J=3.5 Hz, 1H), 3.87 (s, 2H), 3.76 (s, 3H).

Synthesis of 2-(5-chloro-2-cyanophenyl)acetic acid. Lithium hydroxide (44 mg, 1.05 mmol) was added to a mixture of methyl 2-(5-chloro-2-cyanophenyl)acetate (200 mg, 0.95 mmol), in THF (10 mL), water (1 mL) and the mixture stirred at room temperature for 90 min. The mixture was acidified to pH 3 using HCl (2N, aq.) and then extracted with DCM. The organic phase was passed through a phase separator cartridge and concentrated in vacuo to give the title compound (200 mg, quant.) which was used without further purification.

Synthesis of 2-(5-chloro-2-cyanophenyl)acetamide. EDC.HCl (160 mg, 0.84 mmol) was added to a mixture of 2-(5-chloro-2-cyanophenyl)acetic acid (150 mg, 0.77 mmol), HOBt (110 mg, 0.84 mmol), DIPEA (0.6 mL, 3.5 mmol), (NH₄)₂CO₃ in THF (4.2 mL), DMF (0.6 mL). The resulting mixture was heated to 50° C. for 2.5 h. The mixture was then diluted with water (30 mL), DCM (60 mL) and passed through a phase separator cartridge. The organic phase was concentrated in vacuo and purified by column chromatography on silica gel, eluting with a gradient of 0-10% MeOH in DCM to give the title compound (90 mg, 60%). ESI-MS (M+H)⁺: 195.1, ¹H NMR (400 MHz, CDCl₃) δ7.60 (d, J=8.4 Hz, 1H), 7.55 (d, J=2.0 Hz, 1H), 7.41-7.37 (m, 1H), 5.67 (s, 1H), 5.45 (s, 1H), 3.76 (s, 2H).

### Synthesis of 2-((3-chlorophenyl)(methyl)amino)propanamide

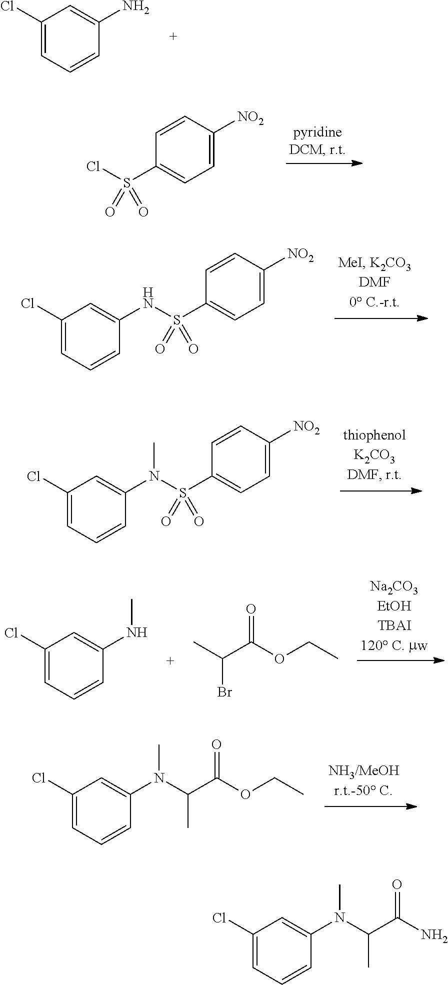

Synthesis of N-(3-chlorophenyl)-4-nitrobenzenesulfonamide. A mixture of 3-chloroaniline (0.83 mL, 7.8 mmol), 4-nitrobenzenesulfonyl chloride (1.7 g, 7.8 mmol) and pyridine (0.70 mL, 8.6 mmol) in DCM (40 mL) was stirred at room temperature for 18 h. Water (100 mL) was added and the mixture was extracted with DCM (3×100 mL). The combined organic layers were dried (MgSO₄), filtered and concentrated in vacuo. The residue was purified by column chromatography on silica gel, eluting with 0-60% EtOAc in cyclohexane to give the title compound (2.4 g, 97%) as a yellow solid. ESI-MS (M+H)⁺: 311.0.

Synthesis of N-(3-chlorophenyl)-N-methyl-4-nitrobenzenesulfonamide. Methyl iodide (0.57 mL, 9.2 mmol) was added to a stirred mixture of N-(3-chlorophenyl)-4-nitrobenzenesulfonamide (2.4 g, 7.7 mmol) and K₂CO₃ (1.6 g, 12 mmol) in DMF (20 mL) at 0° C. under a N₂ atmosphere. The mixture was warmed to room temperature and stirred for 3 h. Water (100 mL) and brine (sat. aq., 100 mL) were added and the mixture was extracted with DCM (100 mL). The organic layer was dried (MgSO₄), filtered and concentrated in vacuo to give the title compound (2.6 g, quant.). ¹H NMR (400 MHz, DMSO) δ8.45-8.40 (m, 2H), 7.84-7.81 (m, 2H), 7.43-7.41 (m, 2H), 7.30 (d, J=1.6 Hz, 1H), 7.16-7.12 (m, 1H), 3.22 (s, 3H).

Synthesis of 3-chloro-N-methylaniline. A mixture of N-(3-chlorophenyl)-N-methyl-4-nitrobenzenesulfonamide (2.5 g, 7.7 mmol), thiophenol (1.6 mL, 15 mmol) and K₂CO₃ (2.6 g, 19 mmol) in DMF (20 mL) was stirred at room temperature under a N₂ atmosphere for 72 h. Water (50 mL) and brine (sat. aq., 50 mL) were added and the mixture was extracted with EtOAc (3×100 mL). The combined organic layers were dried (MgSO₄), filtered and concentrated in vacuo. The residue was purified by column chromatography on silica gel, eluting with 0-60% EtOAc in cyclohexane. The residue was further purified by loading onto an SCX cartridge, washing with MeOH in DCM and then eluting with NH₃ in MeOH in DCM to give the title compound (0.84 g, 78%) as a yellow oil. ESI-MS (M+H)⁺: 142.1, ¹H NMR (400 MHz, DMSO) δ7.10-7.05 (m, 1H), 6.54-6.47 (m, 3H), 6.00-5.93 (m, 1H), 2.67 (d, J=5.0 Hz, 3H).

Synthesis of ethyl N-(3-chlorophenyl)-N-methylalaninate. A mixture of 3-chloro-N-methylaniline (0.84 g, 5.9 mmol), ethyl 2-bromopropanoate (0.85 mL, 6.5 mmol) and Na₂CO₃ (0.94 g, 8.9 mmol) in EtOH (12 mL) was stirred at room temperature for 5 min then sealed and heated at 100° C. for 15 min in a microwave. The mixture was then unsealed and heated at reflux for 18 h. The mixture was resealed and heated at 100° C. in a microwave for 1 h then at 150° C. for further 1 h in a microwave. TBAI (1.3 g, 3.5 mmol) was added and the mixture was stirred at 150° C. in a microwave for 4.5 h. Water (100 mL) was added and the mixture was extracted with EtOAc (3×100 mL). The combined organic layers were dried (MgSO₄), filtered and concentrated in vacuo. The residue was purified by column chromatography on silica gel, eluting with 0-60% EtOAc in cyclohexane. The residue was further purified by loading onto an SCX cartridge, washing with MeOH in DCM and then eluting with NH₃ in MeOH in DCM. The residue was further purified by column chromatography on silica gel, eluting with 0-40% EtOAc in cyclohexane to give the title compound (0.40 g, 28%) as a colourless oil. ESI-MS (M+H)⁺: 242.2, ¹H NMR (400 MHz, DMSO) δ7.21-7.16 (m, 1H), 6.80-6.77 (m, 1H), 6.76-6.72 (m, 1H), 6.71-6.68 (m, 1H), 4.72 (q, J=7.0 Hz, 1H), 4.16-4.06 (m, 2H), 2.79 (s, 3H), 1.39 (d, J=7.0 Hz, 3H), 1.17 (t, J=7.1 Hz, 3H).

Synthesis of 2-((3-chlorophenyl)(methyl)amino)propenamide. Ethyl N-(3-chlorophenyl)-N-methylalaninate (100 mg, 0.41 mmol) was added to a reaction tube containing NH₃ (7.0 N in MeOH, 0.59 mL). The tube was sealed and stirred at room temperature for 18 h. Further NH₃ (7.0 N in MeOH, 0.59 mL) was added and the mixture was heated to 50° C. for a further 18 h. The mixture was concentrated in vacuo and purified by column chromatography on silica gel, eluting with a gradient of 0-10% MeOH in DCM to give the title compound (28 mg, 32%). ESI-MS (M+H)⁺: 213.1, ¹H NMR (400 MHz, DMSO) δ7.40 (s, 1H), 7.22 (dd, J=8.1, 8.1 Hz, 1H), 7.12 (s, 1H), 6.81-6.70 (m, 3H), 4.40 (q, J=6.9 Hz, 1H), 2.86 (s, 3H), 1.31 (d, J=7.1 Hz, 3H).

### Synthesis of 2-(6-cyano-2-fluoro-3-methoxyphenyl)acetamide

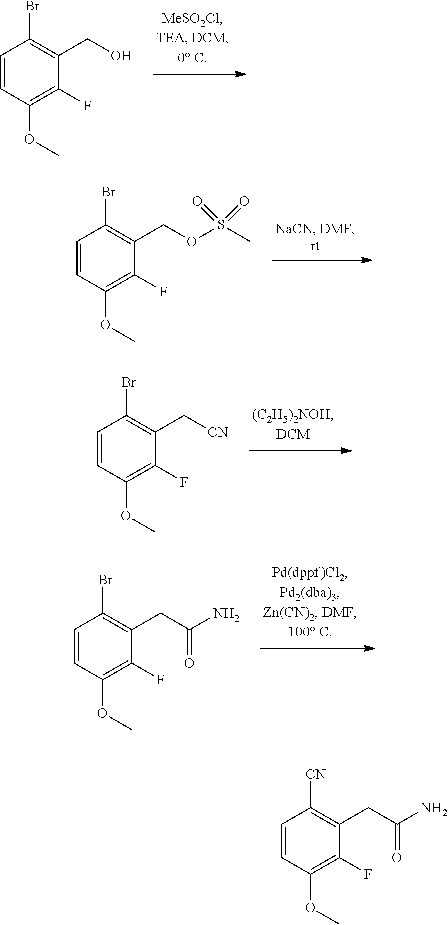

Synthesis of 6-bromo-2-fluoro-3-methoxybenzyl methanesulfonate. Methanesulfonyl chloride (0.43 mL, 5.62 mmol) was added to cooled mixture of (6-bromo-2-fluoro-3-methoxyphenyl)methanol (1.2 g, 5.11 mmol) and TEA (1.1 mL, 7.66 mmol) in DCM (30 mL) at 0° C. The mixture was allowed to warm to room temperature and stirred for 90 min. The mixture was then treated with NaHCO₃ (aq. sat. 30 mL) and extracted with DCM (100 mL). The organic phase was passed through a phase separator cartridge and concentrated in vacuo to give the title compound (1.6 g, quant.) as a brown oil. ¹H NMR (400 MHz, CDCl₃) δ7.36 (dd, J=2.0, 8.8 Hz, 1H), 6.96-6.91 (m, 1H), 5.41-5.40 (m, 2H), 3.90 (s, 3H), 3.07 (s, 3H).

Synthesis of 2-(6-bromo-2-fluoro-3-methoxyphenyl)acetonitrile. Sodium cyanide (500 mg, 10.20 mmol) was added to a solution of 6-bromo-2-fluoro-3-methoxybenzyl methanesulfonate (1.597 g, 5.10 mmol) in DMF (12 mL) and stirred at room temperature for 18 h. The mixture was diluted with water (50 mL), extracted with EtOAc (300 mL), dried (MgSO₄), filtered and concentrated in vacuo. Purification by column chromatography on silica gel, eluting with a gradient of 10-20% EtOAc in cyclohexane gave the title compound (1.2 g, 95%) as a white solid. ¹H NMR (400 MHz, CDCl₃) δ7.35 (dd, J=2.0, 8.9 Hz, 1H), 6.91-6.86 (m, 1H), 3.89 (s, 3H), 3.86 (d, J=2.1 Hz, 2H).

Synthesis of 2-(6-bromo-2-fluoro-3-methoxyphenyl)acetamide. N,N-Diethylhydroxylamine (0.25 mL, 2.46 mmol) was added to a solution of 2-(6-bromo-2-fluoro-3-methoxyphenyl)acetonitrile (150 mg, 0.615 mmol) in DCM (3 mL). The resulting mixture was warmed to 40° C. for 22 h. The mixture was then diluted with water (5 mL), DCM (70 mL) and the phases were separated using a phase separator cartridge. The organic phase was concentrated in vacuo to give the title compound (153 mg, 95%). ¹H NMR (400 MHz, CDCl₃) δ7.36-7.30 (m, 1H), 6.83 (t, J=8.8 Hz, 1H), 5.40 (s, 2H), 3.88 (s, 3H), 3.80 (d, J=2.5 Hz, 2H).

Synthesis of 2-(6-cyano-2-fluoro-3-methoxyphenyl)acetamide. Zinc cyanide (69 mg, 0.588 mmol) was added to a nitrogen purged mixture of 2-(6-bromo-2-fluoro-3-methoxyphenyl)acetamide (140 mg, 0.534 mmol), Pd(dppf)Cl₂ (20 mg, 0.027 mmol), Pd₂(dba)₃ (24 mg, 0.027 mmol) in DMF (3 mL). The mixture was heated to 80° C. for 60 h, an additional amount of Pd(dppf)Cl₂ (20 mg, 0.027 mmol), was added and the mixture was heated to 100° C. for a further 18 h. The mixture was diluted with water (30 mL), the solid was removed by filtration and the aqueous phase was extracted with EtOAc (120 mL). The organic phases were combined, dried (MgSO₄), filtered and concentrated in vacuo. The residue was purified by column chromatography on silica gel, eluting with a gradient of 0-10% MeOH in DCM to give the title compound (38 mg, 34%) as a white solid. ESI-MS (M+H)⁺: 209.1

### Synthesis of 2-(5-chloro-2-(1H-tetrazol-1-yl)phenyl)acetamide

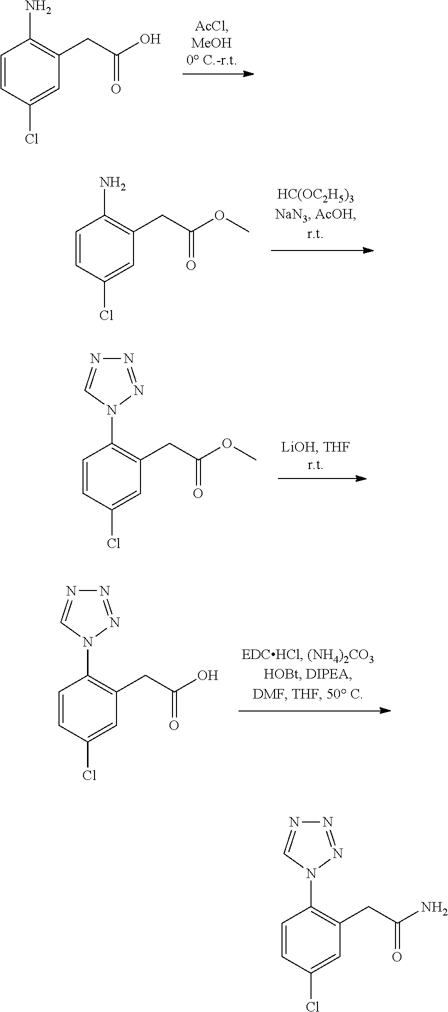

Synthesis of methyl 2-(2-amino-5-chlorophenyl)acetate. Acetyl chloride (5.5 mL, 77 mmol) was added to an ice cooled solution of MeOH (55 mL). After 5 min 2-(2-amino-5-chlorophenyl)acetic acid (1.0 g, 5.39 mmol) was added portion wise. The mixture was stirred at room temperature for 2 h and concentrated in vacuo to give the title compound which was used without further purification. ¹H NMR (400 MHz, DMSO) δ7.44-7.37 (m, 2H), 7.34-7.29 (m, 1H), 4.51 (br s, 2H), 3.88 (s, 2H), 3.68 (s, 3H).

Synthesis of methyl 2-(5-chloro-2-(1H-tetrazol-1-yl)phenyl)acetate. Sodium azide (1.05 g, 16.17 mmol) was added to a mixture of methyl 2-(2-amino-5-chlorophenyl)acetate (1.08 g, 5.39 mmol), triethyl orthoformate (1.8 mL, 16.17 mmol) in AcOH (30 mL) and stirred at room temperature for 18 h. The mixture was diluted with water (60 mL), the precipitate formed was filtered off and the aqueous layer was extracted with DCM. The organic phase was concentrated in vacuo. Purification by column chromatography on silica gel, eluting with a gradient of 0-50% EtOAc in cyclohexane gave the title compound (248 mg, 46%). ¹H NMR (400 MHz, CDCl₃) δ8.92-8.90 (m, 1H), 7.51-7.48 (m, 2H), 7.35-7.32 (m, 1H), 3.64 (s, 3H), 3.57 (s, 2H).

Synthesis of 2-(5-chloro-2-(1H-tetrazol-1-yl)phenyl)acetic acid. Lithium hydroxide (1N, aq., 2.4 mL, 2.37 mmol) was added to a solution of methyl 2-(5-chloro-2-(1H-tetrazol-1-yl)phenyl)acetate (300 mg, 1.19 mmol) in THF (10 mL) and the mixture stirred at room temperature for 3 h. The mixture was diluted with water and extracted with DCM. The aqueous phase was acidified with HCl (2N, aq.) and extracted with EtOAc (×3). The organic phases were combined, dried (MgSO₄) and concentrated in vacuo to give the title compound (252 mg, 89%)¹H NMR (400 MHz, DMSO) δ 12.54 (s, 1H), 9.86 (s, 1H), 7.79 (s, 1H), 7.71 (s, 2H), 3.73-3.70 (m, 2H).

Synthesis of 2-(5-chloro-2-(1H-tetrazol-1-yl)phenyl)acetamide. EDC.HCl (96 mg, 0.503 mmol) was added to a mixture of HOBt (62 mg, 0.461 mmol), DIPEA (0.33 mL, 1.89 mmol), (NH₄)₂CO₃ (181 mg, 1.89 mmol) and 2-(5-chloro-2-(1H-tetrazol-1-yl)phenyl)acetic acid (100 mg, 0.419 mmol) in THF (3 mL), DMF (0.4 mL) and the resulting mixture heated to 50° C. for 3 h. The mixture was diluted with water (10 mL), extracted with EtOAc (3×50 mL), the combined organic phases were dried (MgSO₄) and concentrated in vacuo. The residue was triturated with DCM/DIPE (1:1, 1 mL) to give the title compound (70 mg, 70%) as a white solid. ESI-MS (M+H)⁺: 238.1, ¹H NMR (400 MHz, DMSO) δ9.77 (s, 1H), 7.70 (d, J=1.4 Hz, 1H), 7.63-7.62 (m, 2H), 7.42-7.40 (m, 1H), 6.92-6.90 (m, 1H), 3.44 (s, 2H).

### Synthesis of 2-(3-fluoro-4-hydroxypyridin-2-yl)acetamide

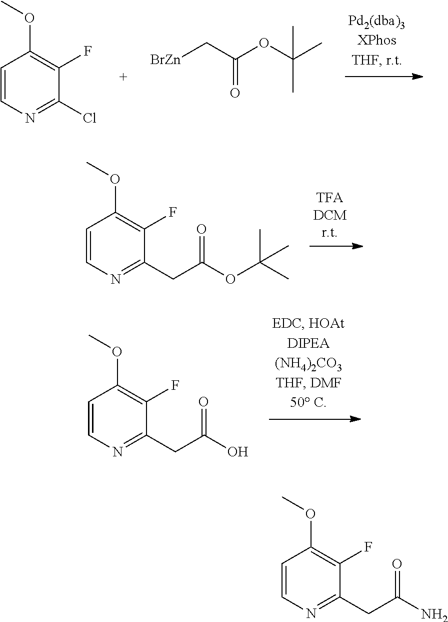

Synthesis of tert-butyl 2-(3-fluoro-4-methoxypyridin-2-yl)acetate. Pd₂(dba)₃ (40 mg, 0.433 mmol) was added to a nitrogen purged mixture of 2-chloro-3-fluoro-4-methoxypyridine (700 mg, 4.33 mol), XPhos (410 mg, 0.87 mmol), (2-(tert-butoxy)-2-oxoethyl)zinc(II) bromide (1M, 6.5 mL, 6.5 mmol) in THF (7 mL) and the resultant mixture stirred at room temperature for 18 h. The mixture was diluted with NH₄Cl (aq. sat. 5 mL), NaHCO₃ (aq. sat. 50 mL) and extracted with EtOAc (3×50 mL). The organic phases were combined, dried (MgSO₄), filtered and concentrated in vacuo. Purification by column chromatography on silica gel, eluting with a gradient of 0-100% EtOAc in cyclohexane gave the title compound (98 mg g, 9.4%) as a yellow oil. ESI-MS (M+H)⁺: 242.2, ¹H NMR (400 MHz, DMSO) δ8.21 (d, J=5.6 Hz, 1H), 7.20 (dd, J=5.6, 6.7 Hz, 1H), 3.93 (s, 3H), 3.75 (d, J=2.9 Hz, 2H), 1.40 (s, 9H).

Synthesis of 2-(3-fluoro-4-methoxypyridin-2-yl)acetic acid. Trifluoroacetic acid (0.5 mL, 6.53 mmol) was added to a solution of tert-butyl 2-(3-fluoro-4-methoxypyridin-2-yl)acetate (120 mg, 0.497 mmol) in DCM (3 mL) and the reaction was stirred at room temperature for 18 h. The mixture was concentrated in vacuo and taken on to the next stage without further purification.

Synthesis of 2-(3-fluoro-4-methoxypyridin-2-yl)acetamide. Crude 2-(3-fluoro-4-methoxypyridin-2-yl)acetic acid (92 mg, 0.450 mmol) was added to a mixture of EDC.HCl (110 mg, 0.60 mmol), HOAt (74 mg, 0.55 mmol), DIPEA (0.39 mL, 2.24 mmol), (NH₄)₂CO₃ (210 mg, 2.24 mmol) in THF (4 mL), DMF (1 mL) and the mixture heated to 50° C. for 4 h. Additional amounts of DIPEA (0.39 mL, 2.24 mmol), (NH₄)₂CO₃ (210 mg, 2.24 mmol) were added and the mixture was stirred at 50° C. for 18 h. Additional amounts of (NH₄)₂CO₃ (500 mg, 5.20 mmol) and DMAP (catalytic) were added and the mixture was stirred at 60° C. for 18 h. The mixture was diluted with water (10 mL) and extracted with EtOAC (3×20 mL). The organic phases were combined, dried (MgSO₄) and concentrated in vacuo. Purification by column chromatography on silica gel, eluting with a gradient of 0-20% 7 N NH₃ in MeOH in DCM gave the title compound (35 mg, 35%) as a white solid. ESI-MS (M+H)⁺: 185.1, ¹H NMR (400 MHz, DMSO) δ8.23 (d, J=5.6 Hz, 1H), 7.55 (s, 1H), 7.20 (q, J=4.0 Hz, 1H), 7.03 (s, 1H), 3.96 (s, 3H), 3.65 (d, J=3.0 Hz, 2H).

### Synthesis of 1-amino-6,7-dihydro-5H-cyclopenta[c]pyridine-6-carboxylic acid hydrochloride

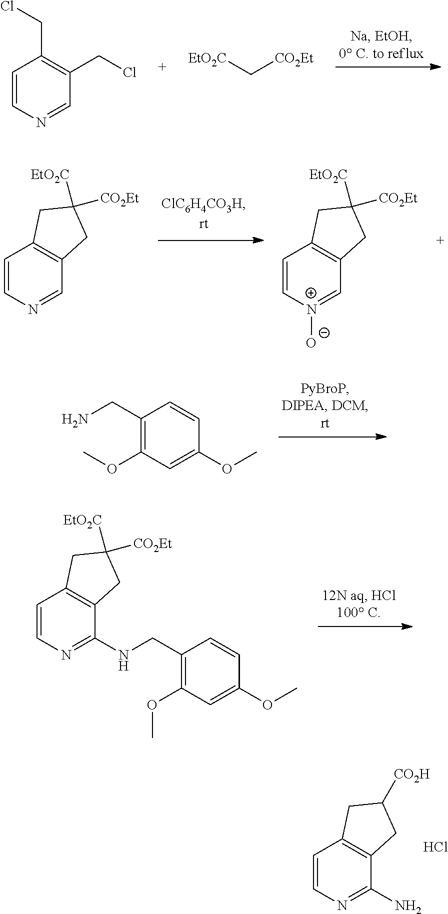

Synthesis of diethyl 5,7-dihydro-6H-cyclopenta[c]pyridine-6,6-dicarboxylate. Sodium (1.06 g, 46.11 mmol) was added portion wise to ethanol (100 mL) and stirred for 1 h. Diethyl malonate (2.3 mL, 15.37 mmol) was then added dropwise over 5 min, followed by dropwise addition of 3,4-bis(chloromethyl)pyridine (2.71 g, 15.37 mmol) in EtOH (20 mL) at room temperature. The mixture was heated to reflux for 4.5 h and then concentrated in vacuo. Water was added and the mixture was extracted with EtOAc. The organic phase was dried (MgSO₄), filtered and concentrated in vacuo. Purification by column chromatography on silica gel, eluting with a gradient of 0-60% EtOAc in cyclohexane gave the title compound (2.07 g, 51%) as an orange oil. ¹H NMR (400 MHz, CDCl₃) δ 8.50-8.37 (m, 2H), 7.16 (d, J=4.8 Hz, 1H), 4.25-4.19 (m, 4H), 3.63 (s, 2H), 3.60 (s, 2H), 1.29-1.24 (m, 6H).

Synthesis of 6,6-bis(ethoxycarbonyl)-6,7-dihydro-5H-cyclopenta[c]pyridine 2-oxide. A solution of 3-chloroperbenzoic acid (2.58 g, 10.37 mmol) in DCM (20 mL) was passed through a phase separator cartridge, and was subsequently added dropwise to a mixture of diethyl 5,7-dihydro-6H-cyclopenta[c]pyridine-6,6-dicarboxylate (1.82 g, 6.91 mmol) in DCM (70 mL) at room temperature and stirred for 18 h. The mixture was washed with Na₂CO₃ (10% aq. 3×), dried (MgSO₄) and concentrated in vacuo to give the title compound (1.77 g, 92%) as an orange solid. ¹H NMR (400 MHz, DMSO) δ 8.22 (s, 1H), 8.09-8.06 (m, 1H), 7.35-7.32 (m, 1H), 4.25-4.18 (m, 4H), 3.53 (s, 2H), 3.50 (s, 2H), 1.25-1.21 (m, 6H).

Synthesis of diethyl 1-((2,4-dimethoxybenzyl)amino)-5,7-dihydro-6H-cyclopenta[c]pyridine-6,6-dicarboxylate. PyBroP® (3.84 g, 8.24 mmol) was added to a mixture of 6,6-bis(ethoxycarbonyl)-6,7-dihydro-5H-cyclopenta[c]pyridine 2-oxide (1.77 g, 6.34 mmol), 2.4-dimethoxybenzylamine (1.32 g, 7.92 mmol) and DIPEA (4.1 mL, 23.77 mmol) in DCM (30 mL) and stirred at room temperature for 18 h. The mixture was then poured into NaHCO₃ (aq. sat.) and extracted with DCM (×3). The organic phases were combined, dried (MgSO₄) and concentrated in vacuo. Purification by column chromatography on silica gel, eluting with a gradient of 0-40% EtOAc in cyclohexane gave the title compound (1.05 g, 39%) as an orange oil. ¹H NMR (400 MHz, CDCl₃) δ 8.01-7.98 (m, 1H), 7.27-7.24 (m, 1H), 6.50-6.42 (m, 3H), 4.60-4.57 (m, 2H), 4.39-4.34 (m, 1H), 4.24-4.17 (m, 4H), 3.84 (s, 3H), 3.80 (s, 3H), 3.52 (s, 2H), 3.32-3.30 (m, 2H), 1.27-1.23 (m, 6H).

Synthesis of 1-amino-6,7-dihydro-5H-cyclopenta[c]pyridine-6-carboxylic acid hydrochloride. A mixture of diethyl 1-((2,4-dimethoxybenzyl)amino)-5,7-dihydro-6H-cyclopenta[c]pyridine-6,6-dicarboxylate (1.08 g, 2.52 mmol) in HCl (12 N, aq, 5 mL) was heated to 100° C. for 6 h. The mixture was concentrated in vacuo and triturated with diethyl ether, filtered, dried under vacuo to give the title compound. ¹H NMR (400 MHz, DMSO) δ13.51 (s, 1H), 12.61 (s, 1H), 7.97 (s, 2H), 7.87 (d, J=6.6 Hz, 1H), 6.92 (d, J=6.3 Hz, 1H), 3.81-3.05 (m, 5H).

### Synthesis of 2-(6-((diphenylmethylene)amino)-2-fluoro-3-methoxyphenyl)acetamide

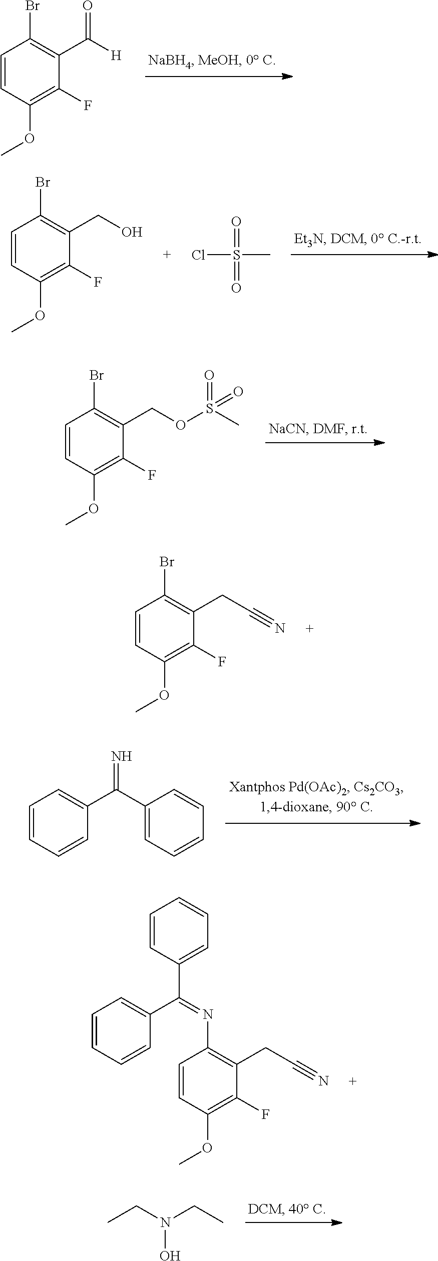

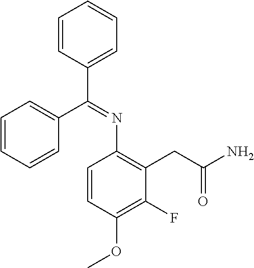 [SMILES: COc1ccc(N=C(c2ccccc2)c2ccccc2)c(CC(N)=O)c1F]

Synthesis of (6-bromo-2-fluoro-3-methoxyphenyl)methanol. A solution of 6-bromo-2-fluoro-3-methoxybenzaldehyde (1.4 g, 5.9 mmol) in MeOH (16 mL) was cooled to 0° C. Sodium borohydride (220 mg, 5.9 mmol) was added portion wise and the reaction mixture was stirred at 0° C. for 40 min. The reaction mixture was allowed to warm up to room temperature and concentrated in vacuo. The residue was dissolved in EtOAc (130 mL) and washed with water (30 mL) and brine (sat. aq., 20 mL). The organics were dried over MgSO₄ and concentrated in vacuo to give the title compound (1.3 g, 96%) as a colourless oil which was used without further purification.

Synthesis of 6-bromo-2-fluoro-3-methoxybenzyl methanesulfonate. To a solution of (6-bromo-2-fluoro-3-methoxyphenyl)methanol (1.3 g, 5.7 mmol) in DCM (35 mL) was added triethylamine (1.2 mL, 8.5 mmol) and the reaction cooled to 0° C. Methanesulfonyl chloride (0.48 mL, 6.2 mmol) was added and the reaction mixture was allowed to warm up to room temperature and stirred for 1 h. The reaction mixture was diluted with NaHCO₃ (sat. aq., 35 mL) and extracted with DCM (2×60 mL). The organics were dried over a hydrophobic frit and concentrated in vacuo to give the title compound (970 mg, 54%) as a colourless oil. ¹H NMR (400 MHz, CDCl₃) δ7.39-7.34 (m, 1H), 6.97-6.90 (m, 1H), 5.41 (d, J=2.2 Hz, 2H), 3.90 (s, 3H), 3.07 (s, 3H).

Synthesis of 2-(6-bromo-2-fluoro-3-methoxyphenyl)acetonitrile. Sodium cyanide (300 mg, 6.2 mmol) was added to a solution of 6-bromo-2-fluoro-3-methoxybenzyl methanesulfonate (970 mg, 3.1 mmol) in DMF (10 mL) and the reaction mixture was then stirred at room temperature for 20 h. The reaction mixture was diluted with water (50 mL) and extracted with EtOAc (200 mL). The organics were washed with brine (sat. aq., 100 mL), dried over MgSO₄ and concentrated in vacuo. The residue was purified by column chromatography on silica gel, eluting with a gradient of 0-20% EtOAc in isohexane to give the title compound (1.1 g, quant.) as a white solid. ESI-MS (M−H)−: 242.1, 244.0, ¹H NMR (400 MHz, CDCl₃) δ7.35 (dd, J=2.0, 8.9 Hz, 1H), 6.88 (t, J=8.8 Hz, 1H), 3.89 (s, 3H), 3.86 (d, J=2.0 Hz, 2H).

Synthesis of 2-(6-((diphenylmethylene)amino)-2-fluoro-3-methoxyphenyl)acetonitrile. A mixture of 2-(6-bromo-2-fluoro-3-methoxyphenyl)acetonitrile (930 mg, 3.8 mmol), diphenylmethanimine (0.96 mL, 5.7 mmol) and Cs₂CO₃ (3.7 g, 11 mmol) was suspended in anhydrous 1,4-dioxane (15 mL) and degassed for 5 min. Xantphos (220 mg, 0.38 mmol) and Pd(OAc)₂ (43 mg, 0.19 mmol) were added and the reaction mixture was degassed for a further 5 min, then stirred at 90° C. for 3.5 h. The reaction mixture was allowed to cool to room temperature, diluted with EtOAc (250 mL), washed with water (100 mL) and brine (50 mL). The organics were dried over MgSO₄ and concentrated in vacuo. The residue was purified by column chromatography on silica gel, eluting with a gradient of 0-100% DCM in isohexane to give the title compound (550 mg, 42%) as a yellow solid. ESI-MS (M+H)⁺: 345.1.

Synthesis of 2-(6-((diphenylmethylene)amino)-2-fluoro-3-methoxyphenyl)acetamide. A mixture of 2-(6-((diphenylmethylene)amino)-2-fluoro-3-methoxyphenyl)acetonitrile (100 mg, 0.29 mmol) and N,N-diethylhydroxylamine (0.18 mL, 1.7 mmol) in DCM (2.0 mL) was placed under a N₂ atmosphere and the reaction mixture was stirred at 40° C. for 24 h. The reaction mixture was allowed to cool to room temperature and was partitioned between DCM (50 mL) and water (10 mL). The organic phase was dried over a hydrophobic frit and concentrated in vacuo. The residue was purified by column chromatography on silica gel, eluting with a gradient of 0-25% EtOAc in DCM to give the title compound (98 mg, 93%) as a yellow solid. ESI-MS (M+H)⁺: 363.2, ¹H NMR (400 MHz, CDCl₃) δ7.72 (d, J=7.3 Hz, 2H), 7.52-7.28 (m, 6H), 7.18-7.12 (m, 2H), 6.59-6.53 (m, 1H), 6.47 (s, 1H), 6.07 (d, J=8.3 Hz, 1H), 5.25 (s, 1H), 3.78 (s, 3H), 3.71 (s, 2H).

### Synthesis of 2-(7-chloroimidazo[1,5-a]pyridin-1-yl)-N-(6-chloropyrimidin-4-yl)acetamide

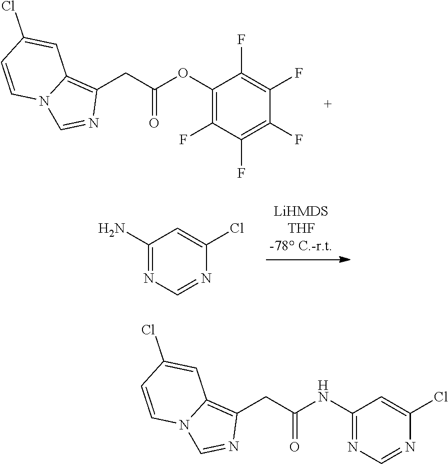

LiHMDS (1.0 M in THF, 0.66 mL, 0.66 mmol) was added to a stirred solution of 6-chloropyrimidin-4-amine (0.085 g, 0.66 mmol) in THF (2.4 mL) at −78° C. under a N₂ atmosphere. After 45 min at −78° C. a solution of perfluorophenyl 2-(7-chloroimidazo[1,5-a]pyridin-1-yl)acetate (0.23 g, 0.60 mmol) in THF (4.6 mL) was added slowly. After 30 min at −78° C. the mixture was warmed to room temperature and stirred for 4 h. Na₂CO₃ (10% aq., 75 mL) was then added and the mixture was extracted with EtOAc (3×75 mL). The combined organic layers were dried (MgSO₄), filtered and concentrated in vacuo. The residue was redissolved in DCM, filtered and concentrated in vacuo. The residue was triturated with MeOH (×3) to give the title compound (0.070 g, 36%) as a yellow solid. ESI-MS (M+H)⁺: 322.0, 324.0, ¹H NMR (400 MHz, DMSO) δ11.48 (s, 1H), 8.83 (s, 1H), 8.41-8.34 (m, 2H), 8.13 (s, 1H), 7.88 (s, 1H), 6.75-6.70 (m, 1H), 4.10 (s, 2H).

### Synthesis of 2-(7-chloroimidazo[1,5-a]pyridin-1-yl)-N-(4-chloropyrimidin-5-yl)acetamide

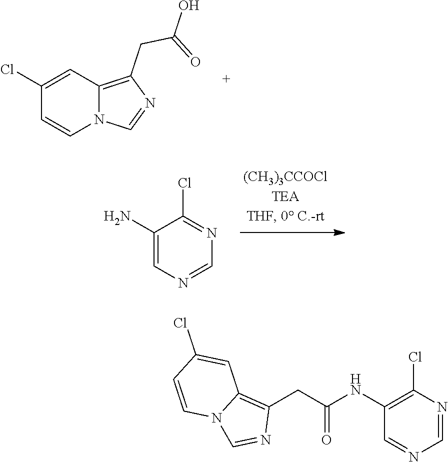

Trimethylacetyl chloride (0.38 mL, 3.09 mmol) was added dropwise to a coole mixture of 2-(7-chloroimidazo[1,5-a]pyridin-1-yl)acetic acid (500 mg, 2.37 mmol) and TEA (0.66 mL, 4.75 mmol) in THF (25 mL) at 0° C. After 2 h 4-chloropyrimidin-5-amine (290 mg, 2.26 mmol) was added. After a further 1 h the mixture was allowed to warm to room temperature and stirred for 18 h. The mixture was then diluted with water, brine (aq. sat.) and extracted with EtOAc. The organic phase was dried (MgSO₄), filtered and concentrated in vacuo. Purification by column chromatography on silica gel, eluting with a gradient of 40-100% EtOAc in cyclohexane gave the title compound (272 mg, 35%) as a brown solid. ¹H NMR (400 MHz, DMSO) δ10.25 (s, 1H), 9.19-9.18 (m, 1H), 8.82 (s, 1H), 8.41 (s, 1H), 8.35 (dd, J=1.0, 7.5 Hz, 1H), 7.88-7.86 (m, 1H), 6.69 (dd, J=2.1, 7.5 Hz, 1H), 4.08 (s, 2H).

### Synthesis of 2-(3-chlorophenoxy)propanoic acid

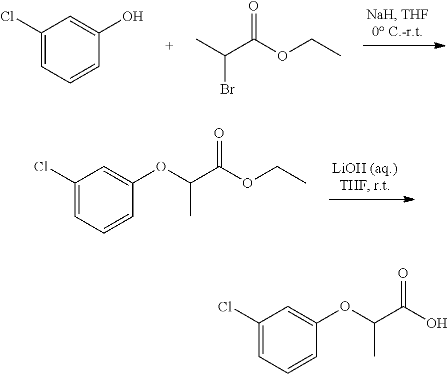

Synthesis of ethyl 2-(3-chlorophenoxy)propanoate. NaH (65% in mineral oil, 0.28 g, 7.0 mmol) was added to a stirred mixture of 3-chlorophenol (0.62 mL, 5.8 mmol) in THF (11 mL) at 0° C. under a N₂ atmosphere. After 15 min ethyl 2-bromopropanoate (0.83 mL, 6.4 mmol) was added at 0° C. After 30 min the mixture was warmed to room temperature and stirred for 18 h. Water (50 mL) and brine (sat. aq., ca. 50 mL) were then added and the mixture was extracted with DCM (2×50 mL) and EtOAc (50 mL). The combined organic layers were dried (MgSO₄), filtered and concentrated in vacuo. The residue was purified by column chromatography on silica gel, eluting with 0-40% EtOAc in cyclohexane to give the title compound (1.6 g, quant.) as a colourless oil. ¹H NMR (400 MHz, DMSO) δ7.34-7.30 (m, 1H), 7.05-7.02 (m, 1H), 6.98 (t, J=2.2 Hz, 1H), 6.90-6.87 (m, 1H), 5.06 (q, J=6.7 Hz, 1H), 4.19-4.13 (m, 2H), 1.52 (d, J=6.8 Hz, 3H), 1.19 (t, J=7.1 Hz, 3H).

Synthesis of 2-(3-chlorophenoxy)propanoic acid. A mixture of ethyl 2-(3-chlorophenoxy)propanoate (1.3 g, 5.8 mmol) and LiOH (1.0 M aq., 12 mL, 12 mmol) in THF (12 mL) was stirred at room temperature for 18 h. Water (50 mL) was added and the pH was adjusted to 4 with HCl (2.0 M aq.). The mixture was extracted with EtOAc (3×50 mL). The aqueous layer pH was adjusted to 1 with HCl (2.0 M aq.) and further extracted with EtOAc (3×50 mL). The combined organic layers were dried (MgSO₄), filtered and concentrated in vacuo to give the title compound (1.4 g, quant) as a white solid. ¹H NMR (400 MHz, DMSO) δ13.03 (s, 1H), 7.32 (t, J=8.2 Hz, 1H), 7.03-7.00 (m, 1H), 6.95 (t, J=2.2 Hz, 1H), 6.89-6.86 (m, 1H), 4.93 (q, J=6.8 Hz, 1H), 1.51 (d, J=6.8 Hz, 3H).

### Synthesis of 2-(3-chlorophenoxy)-N-(6-chloropyrimidin-4-yl)propanamide

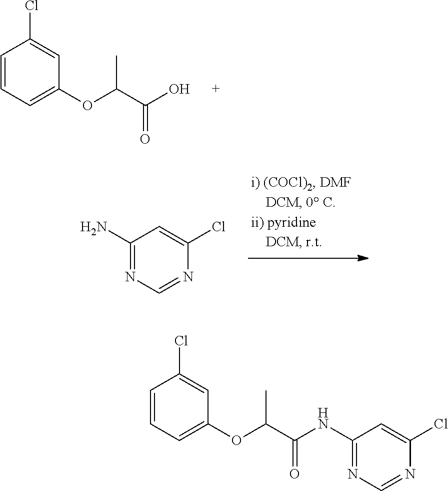 [SMILES: CC(Oc1cccc(Cl)c1)C(=O)O ; Nc1cc(Cl)ncn1 ; CC(Oc1cccc(Cl)c1)C(=O)Nc1cc(Cl)ncn1]

Oxalyl chloride (0.91 mL, 10 mmol) was added to a stirred mixture of 2-(3-chlorophenoxy)propanoic acid (0.70 g, 3.5 mmol) and DMF (2 drops) in DCM (14 mL) at 0° C. under a N₂ atmosphere. The mixture was stirred at 0° C. for 3 h then concentrated in vacuo. The mixture was redissolved in DCM (35 mL) and 6-chloropyrimidin-4-amine (0.45 g, 3.5 mmol) and pyridine (0.42 mL, 5.2 mmol) were added. The mixture was stirred at room temperature for 18 h. Water (25 mL) and brine (sat. aq., 25 mL) were added and the mixture was extracted with DCM (25 mL then 2×50 mL). The combined organic layers were dried (MgSO₄), filtered and concentrated in vacuo. The residue was purified by column chromatography on silica gel, eluting with 0-80% EtOAc in cyclohexane to give the title compound (0.53 g, 49%) as a colourless oil. ¹H NMR (400 MHz, DMSO) δ11.57 (s, 1H), 8.83-8.82 (m, 1H), 8.09-8.08 (m, 1H), 7.36-7.31 (m, 1H), 7.05-7.01 (m, 2H), 6.91-6.88 (m, 1H), 5.15 (q, J=6.6 Hz, 1H), 1.57-1.54 (m, 3H).

### Synthesis of (E)-3-3-chlorophenyl)-N-(6-chloropyrimidin-4-yl)acrylamide

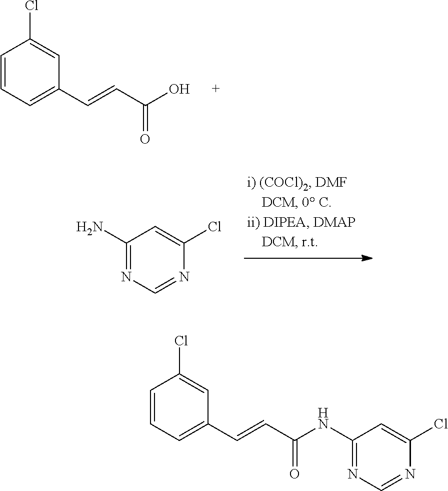 [SMILES: O=C(O)/C=C/c1cccc(Cl)c1 ; Nc1cc(Cl)ncn1 ; O=C(/C=C/c1cccc(Cl)c1)Nc1cc(Cl)ncn1]

Oxalyl chloride (0.26 mL, 3.0 mmol) and DMF (2 drops) were added to a stirred mixture of (E)-3-(3-chlorophenyl)acrylic acid (0.18 g, 1.0 mmol) in DCM (10 mL). The mixture was stirred at room temperature for 2 h then concentrated in vacuo. The mixture was redissolved in DCM (10 mL) and cooled to 0° C. A mixture of 6-chloropyrimidin-4-amine (0.13 g, 1.0 mmol) and DIPEA (0.52 mL, 3.0 mmol) in DCM (2.0 mL) was added portion wise at 0° C. followed by DMAP (0.012 mL, 0.10 mmol). The mixture was then warmed to room temperature and stirred for 3 h. The mixture was then concentrated in vacuo and the residue was purified by column chromatography on silica gel, eluting with 1-10% MeOH in DCM to give the title compound (0.061 g, 21%) as a green cream solid. ESI-MS (M+H)⁺: 293.9.

### Synthesis of 2-(2-bromo-5-chlorophenyl)-N-(6-chloropyrimidin-4-yl)acetamide

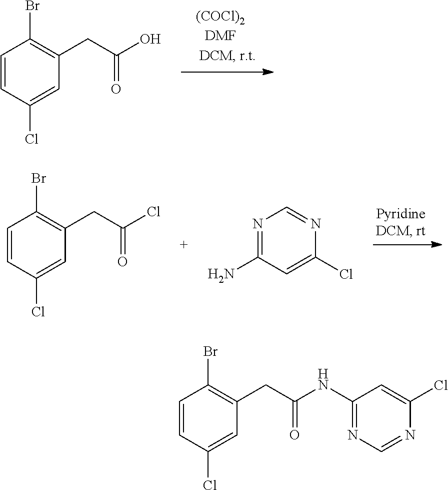

Synthesis of 2-(2-bromo-5-chlorophenyl)acetyl chloride. A solution of 2-(2-bromo-5-chlorophenyl)acetic acid (500 mg, 2.0 mmol) in anhydrous DCM (6.0 mL) was placed under a N₂ atmosphere, oxalyl chloride (0.52 mL, 6.0 mmol) and a few drops of DMF (39 μL, 0.50 mmol) were added and the reaction mixture was stirred at room temperature for 20 minutes. The reaction mixture was concentrated in vacuo to give the title compound which was used directly in the next step without further purification.

Synthesis of 2-(2-bromo-5-chlorophenyl)-N-(6-chloropyrimidin-4-yl)acetamide A solution 2-(2-bromo-5-chlorophenyl)acetyl chloride (536 mg, 2.0 mmol) in DCM (10 mL) was added dropwise to an ice cooled mixture of pyridine (0.24 mL, 3.0 mmol) and 6-chloropyrimidin-4-amine (259 mg, 2.0 mmol) in DCM (10 mL), mixture was allowed to warm to room temperature and stirred for 18 h. The mixture was diluted with water (10 mL), extracted with DCM (100 mL) and the organic phase was passed through a phase separator cartridge and concentrated in vacuo. Purification by column chromatography on silica gel, eluting with a gradient of 0-30% EtOAc in DCM followed by triturating with diethyl ether gave the title compound (48 mg, 7%). ¹H NMR (400 MHz, CDCl₃) δ8.63 (d, J=1.0 Hz, 1H), 8.23 (d, J=1.0 Hz, 1H), 8.01 (s, 1H), 7.58-7.55 (m, 1H), 7.39 (d, J=2.5 Hz, 1H), 7.22 (dd, J=2.6, 8.7 Hz, 1H), 3.89 (s, 2H).

### Synthesis of 2-(5-chloro-2-(1-trityl-1H-tetrazol-5-yl)phenyl)acetamide

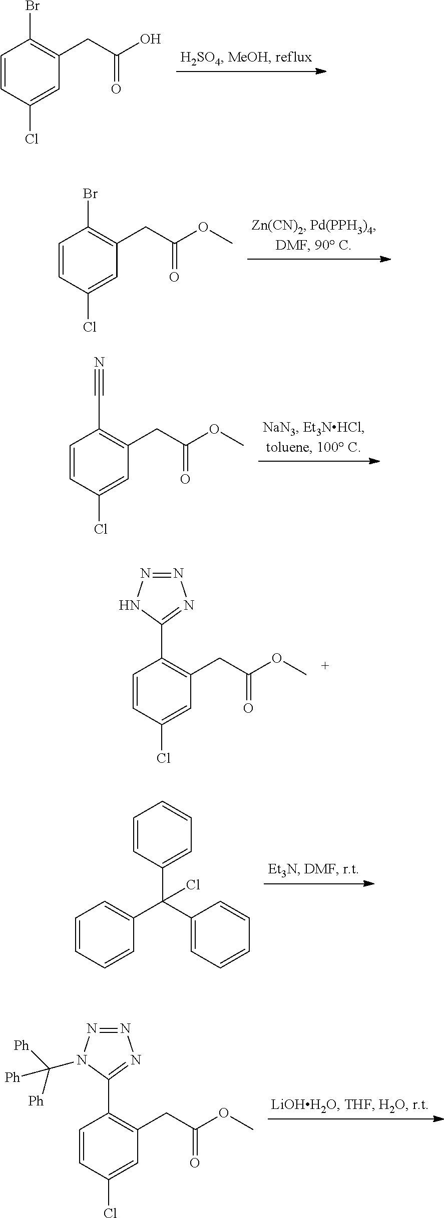

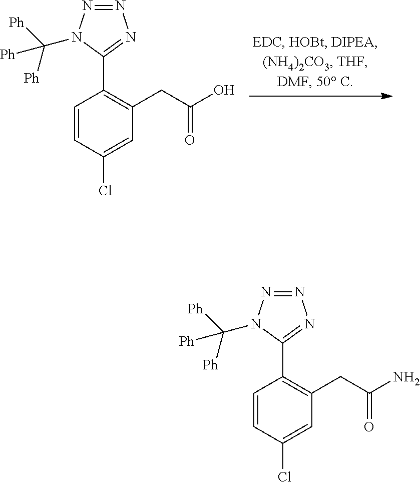 [SMILES: O=C(O)Cc1cc(Cl)ccc1-c1nnnn1C(c1ccccc1)(c1ccccc1)c1ccccc1 ; NC(=O)Cc1cc(Cl)ccc1-c1nnnn1C(c1ccccc1)(c1ccccc1)c1ccccc1]

Synthesis of methyl 2-(2-bromo-5-chlorophenyl)acetate. To a solution of 2-(2-bromo-5-chlorophenyl)acetic acid (3.0 g, 12 mmol) were added a few drops of sulfuric acid (conc., 64 μL, 1.2 mmol) and the reaction mixture was stirred at reflux for 16 hours. The reaction mixture was allowed to cool to room temperature and concentrated in vacuo. The residue was dissolved in EtOAc (180 mL) and washed with water (50 mL) and brine (sat. aq., 50 mL). The organic phase was dried over MgSO₄ and concentrated in vacuo to give the title compound (3.2 g, quant.) as a colourless oil. ¹H NMR (400 MHz, CDCl₃) δ7.50-7.48 (m, 1H), 7.29 (d, J=2.6 Hz, 1H), 7.13 (dd, J=2.5, 8.5 Hz, 1H), 3.76 (s, 2H), 3.73 (s, 3H).

Synthesis of methyl 2-(5-chloro-2-cyanophenyl)acetate. A solution of methyl 2-(2-bromo-5-chlorophenyl)acetate (3.7 g, 14 mmol) in DMF (28 mL) was degassed for 10 minutes, Zn(CN)₂ (850 mg, 7.2 mmol) and Pd(PPh₃)₄ (320 mg, 0.28 mmol) were added and the mixture was degassed for a further 5 minutes. The reaction mixture was then stirred at 90° C. for 16 hours. The reaction mixture was allowed to cool to room temperature, diluted with water (100 mL) and extracted with Et₂O (3×100 mL). The combined organic layers were washed with water (50 mL), LiCl (4% aq., 40 mL), dried over MgSO₄ and concentrated in vacuo. The residue was purified by column chromatography on silica gel, eluting with a gradient of 0-40% EtOAc in isohexane to give the title compound (2.11 g, 72%) as a colourless oil. ¹H NMR (400 MHz, CDCl₃) δ7.62-7.59 (m, 1H), 7.44 (d, J=1.8 Hz, 1H), 7.39 (dd, J=2.0, 8.2 Hz, 1H), 3.87 (s, 2H), 3.76 (s, 3H).

Synthesis of methyl 2-(5-chloro-2-(1H-tetrazol-5-yl)phenyl)acetate. A mixture of methyl 2-(5-chloro-2-cyanophenyl)acetate (400 mg, 1.9 mmol), NaN₃ (190 mg, 2.9 mmol) and Et₃N·HCl (390 mg, 2.9 mmol) in toluene (10 mL) was stirred at 100° C. for 6 hours. The reaction mixture was allowed to cool to room temperature, diluted with EtOAc (20 mL) and extracted with water (40 mL). The aqueous phase was then acidified to pH 3 using HCl (1M aq.) and extracted with DCM (3×40 mL). The combined organic layers were dried over a hydrophobic frit and concentrated in vacuo. The residue was purified by column chromatography on silica gel, eluting with a gradient of 0-10% MeOH in DCM to give the title compound (110 mg, 22%) as a colourless oil. ¹H NMR (400 MHz, CDCl₃) δ7.96-7.92 (m, 1H), 7.48-7.46 (m, 2H), 3.84 (s, 3H), 3.77 (s, 2H). Acidic proton not observed

Synthesis of methyl 2-(5-chloro-2-(1-trityl-1H-tetrazol-5-yl)phenyl)acetate. A mixture of methyl 2-(5-chloro-2-(1H-tetrazol-5-yl)phenyl)acetate (80 mg, 0.32 mmol), trityl chloride (97 mg, 0.35 mmol) and Et₃N (66 μL, 0.48 mmol) in DMF (1.0 mL) was placed under a N₂ atmosphere and stirred at room temperature for 6 hours. The reaction mixture was diluted with DCM (50 mL) and washed with water (20 mL). The organic phase was dried over a hydrophobic frit and concentrated in vacuo. The residue was combined with another batch (commencing with 2-(5-chloro-2-(1H-tetrazol-5-yl)phenyl)acetate (20 mg, 0.079 mmol)) to give the title compound (220 mg, quant.) which was used without further purification.

Synthesis of 2-(5-chloro-2-(1-trityl-1H-tetrazol-5-yl)phenyl)acetic acid. A mixture of methyl 2-(5-chloro-2-(1-trityl-1H-tetrazol-5-yl)phenyl)acetate (220 mg, 0.44 mmol), LiOH H₂O (20 mg, 0.48 mmol) in THF (10 mL) and water (1.0 mL) was stirred at room temperature for 18 hours. The reaction mixture was concentrated in vacuo, the residue was dissolved in water (10 mL), acidified with HCl (2M aq., 1 mL) and extracted with DCM (80 mL). The organic layer was dried over a hydrophobic frit and concentrated in vacuo to give the title compound (230 mg, quant.) as a colourless oil. ¹H NMR (400 MHz, CDCl₃) δ8.08-8.05 (m, 1H), 7.44-7.27 (m, 10H), 7.15-7.12 (m, 7H), 3.91 (s, 2H). 1 exchangeable proton not observed.

Synthesis of 2-(5-chloro-2-(1-trityl-1H-tetrazol-5-yl)phenyl)acetamide. A mixture of 2-(5-chloro-2-(1-trityl-1H-tetrazol-5-yl)phenyl)acetic acid (230 mg, 0.48 mmol), EDC (100 mg, 0.53 mmol), HOBt (71 mg, 0.53 mmol) and DIPEA (0.38 mL, 2.2 mmol) in THF (5.0 mL) and DMF (0.7 mL) was placed under a N₂ atmosphere, (NH₄)₂CO₃ (210 mg, 2.2 mmol) was added and the reaction mixture was stirred at 50° C. for 6 hours. The reaction mixture was allowed to cool to room temperature, diluted with DCM (50 mL) and washed with water (20 mL). The organic phase was dried over a hydrophobic frit and concentrated in vacuo. The residue was purified by column chromatography on silica gel, eluting with a gradient of 0-10% MeOH in DCM. The residue was further purified by column chromatography on silica gel, eluting with a gradient of 2-5% MeOH in DCM to give the title compound (84 mg, 36%) as a white solid. ¹H NMR (400 MHz, CDCl₃) δ8.08-8.05 (m, 1H), 7.58-7.57 (m, 1H), 7.43-7.28 (m, 10H), 7.17-7.13 (m, 6H), 6.29 (s, 1H), 4.83 (s, 1H), 3.69 (s, 2H).

### Synthesis of 2-(6-chloro-1H-benzo[d][1,2,3]triazol-1-yl)acetic acid

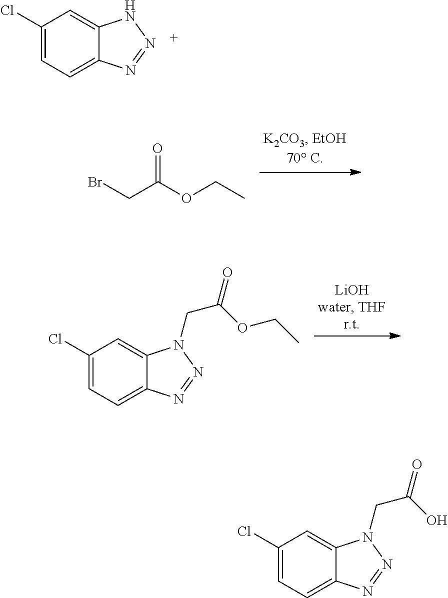

Synthesis of ethyl 2-(6-chloro-1H-benzo[d][1,2,3]triazol-1-yl)acetate. Ethyl bromoacetate (0.43 mL, 3.9 mmol) was added dropwise to a stirred mixture of 6-chloro-1H-benzotriazole (0.5 g, 3.3 mmol) and potassium carbonate (0.9 g, 6.5 mmol) in ethanol (15 mL) and the resulting mixture heated at 70° C. for 2 h. The reaction mixture was cooled to room temperature and filtered. The filtrate was concentrated in vacuo and the residue purified by silica gel column chromatography using a gradient of 2-30% diethylether in DCM as eluant to give the title compound (0.2 g, 26%) as a yellow solid and mixture of regioisomers which was used without further purification.

Synthesis of 2-(6-chloro-1H-benzo[d][1,2,3]triazol-1-yl)acetic acid. A mixture of ethyl 2-(6-chloro-1H-benzo[d][1,2,3]triazol-1-yl)acetate (0.19 g, 0.8 mmol), LiOH (0.038 g, 1.6 mmol) and water (0.028 mL, 1.6 mmol) in THF (5 mL) was stirred at room temperature for 24 hours. The reaction was poured into water (25 mL) and brine (25 mL) and washed with EtOAc (3×20 mL). The aqueous phase was acidified to pH 3 with 10% citric acid and extracted with EtOAc (6×30 mL). The combined organic phase was dried over MgSO₄, and the solvent removed in vacuo to give the title compound (0.134 g, 79%) as a white solid and mixture of regioisomers which was used without further purification.

### Synthesis of 2-(6-chloro-1H-benzo[d][1,2,3]triazol-1-yl)acetic acid

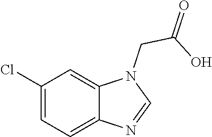 [SMILES: O=C(O)Cn1cnc2ccc(Cl)cc21]

The title compound was prepared as a mixture of regioisomers using a similar method to that described for the synthesis of 2-(6-chloro-1H-benzo[d][1,2,3]triazol-1-yl)acetic acid starting from chlorobenzimidazole (0.337 g, 2.2 mmol) and was used without further purification.

### Synthesis of 6-chloro-N-((7-chloroimidazo[1,5-a]pyridin-1-yl)methyl)pyridazine-4-carboxamide

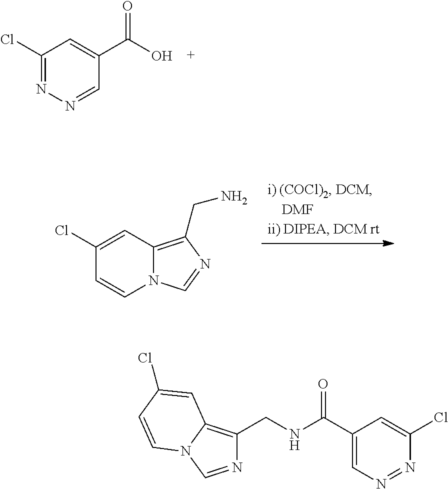 [SMILES: O=C(O)c1cnnc(Cl)c1 ; NCc1ncn2ccc(Cl)cc12 ; O=C(NCc1ncn2ccc(Cl)cc12)c1cnnc(Cl)c1]

Oxalyl chloride (136 μL, 1.56 mmol) was added to a solution of 6-chloropyridazine-4-carboxylic acid (124 mg, 0.780 mmol) in DCM (10 mL) followed by DMF (2 drops) and stirred at room temperature for 3 h. The mixture was concentrated in vacuo, dissolved in DCM (5 mL) and added dropwise to a mixture of (7-chloroimidazo[1,5-a]pyridin-1-yl)methanamine (170 mg, 0.780 mmol, WO2019178129) and DIPEA (543 μL, 3.12 mmol) in DCM (10 mL) at room temperature and the reaction stirred for 18 h. The mixture was concentrated in vacuo, treated with K₂CO₃ (dil. 5 mL), stirred for 30 min and filtered. The solid was washed with water, dried in vacuo (40° C.) to give the title compound (175 mg, 70%) as a brown solid. ESI-MS (M+H)⁺: 323.

### Synthesis of 6-fluoro-N-(2-fluoro-3-methoxy-6-(1H-tetrazol-1-yl)benzyl)pyrimidin-4-amine

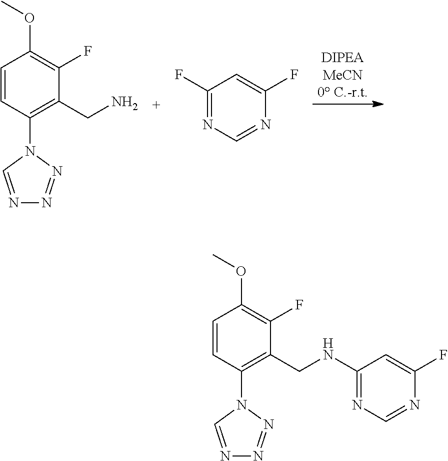 [SMILES: COc1ccc(-n2cnnn2)c(CN)c1F ; Fc1cc(F)ncn1 ; COc1ccc(-n2cnnn2)c(CNc2cc(F)ncn2)c1F]

4,6-Difluoropyrimidine (0.031 g, 0.27 mmol) was added to a stirred solution of (2-fluoro-3-methoxy-6-(1H-tetrazol-1-yl)phenyl)methanamine (0.050 g, 0.22 mmol, WO2017207983) and DIPEA (0.098 mL, 0.56 mmol) in MeCN (0.5 mL) at 0° C. The mixture was stirred at 0° C. for 5 min then warmed to room temperature. After 2.5 h the mixture was diluted with DCM (50 mL), washed with water, passed through a phase separator cartridge and concentrated in vacuo. The residue was purified by column chromatography on silica gel, eluting with 0-100% DCM in EtOAc to give the title compound (0.040 g, 55%) as a white solid. ESI-MS (M+H)⁺: 320, ¹H NMR (400 MHz, CD₃OD) δ 9.49 (s, 1H), 7.99 (d, J=2.5 Hz, 1H), 7.29-7.26 (m, 2H), 5.93 (s, 1H), 5.48 (s, 2H), 3.98-3.97 (m, 3H). 1 exchangeable proton not observed.

### Synthesis of 2-(4-chlorophenyl)cyclopropane-1-sulfonyl chloride

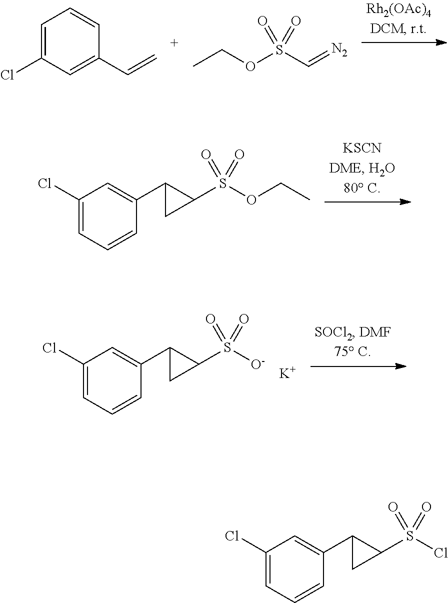

Synthesis of ethyl 2-(3-chlorophenyl)cyclopropane-1-sulfonate. Ethyl diazomethanesulfonate (240 mg, 1.6 mmol) in DCM (6 mL) was added dropwise over 2 h to a solution of 1-chloro-3-vinylbenzene (0.3 mL, 2.40 mmol) in DCM (8 mL) at room temperature and the resulting mixture stirred for 18 h. The mixture was concentrated in vacuo and used in the next step without further purification.

Synthesis of potassium 2-(3-chlorophenyl)cyclopropane-1-sulfonate. Ethyl 2-(3-chlorophenyl)cyclopropane-1-sulfonate (417 mg, 1.60 mmol) was added to a mixture of potassium thiocyanate (163 mg, 1.68 mmol) in 1,2-DME (5 mL) and water (5 mL) and the reaction heated to 80° C. for 4 h. The mixture was diluted with water, washed with EtOAc and the aqueous phase was concentrated under a stream of N₂. The residue was dried under vacuo and used in the next step without further purification. ¹H NMR (400 MHz, DMSO) δ7.34-7.10 (m, 4H), 2.35-2.30 (m, 1H), 2.26-2.20 (m, 1H), 1.34-1.26 (m, 1H), 1.10-1.05 (m, 1H).

Synthesis of 2-(3-chlorophenyl)cyclopropane-1-sulfonyl chloride. A mixture of potassium 2-(3-chlorophenyl)cyclopropane-1-sulfonate (100 mg, 0.369 mmol) and thionyl chloride (1.1 mL, 14.77 mmol) in DMF (0.1 mL) was heated to 75° C. for 4 h and concentrated in vacuo. The residue was dissolved in EtOAc, washed with water, brine, dried (MgSO₄) and concentrated in vacuo to give the title compound which was used directly in the next step without further purification.

### Synthesis of 2-(3-chlorophenyl)-N-(6-chloropyrimidin-4-yl)cyclopropane-1-sulfonamide

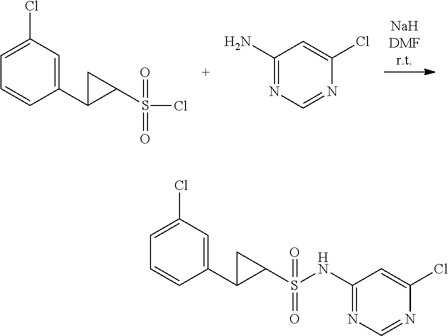 [SMILES: O=S(=O)(Cl)C1CC1c1cccc(Cl)c1 ; Nc1cc(Cl)ncn1 ; O=S(=O)(Nc1cc(Cl)ncn1)C1CC1c1cccc(Cl)c1]

NaH (60% in mineral oil, 0.075 g, 0.19 mmol) was added to a stirred solution of 6-chloropyrimidin-4-amine (0.024 g, 0.19 mmol) in DMF (1.5 mL). After 10 min a solution of 2-(3-chlorophenyl)cyclopropane-1-sulfonyl chloride (0.047 g, 0.19 mmol) in DMF (0.5 mL) was added dropwise. The mixture was stirred at room temperature for 18 h. The mixture was quenched with water and extracted with EtOAc (3×). The combined organic layers were dried (MgSO₄), filtered and concentrated in vacuo to give the title compound as a brown gum, which was used directly without further purification.

### Synthesis of (6-cyclopropylimidazo[1,2-a]pyridin-2-yl)methanamine

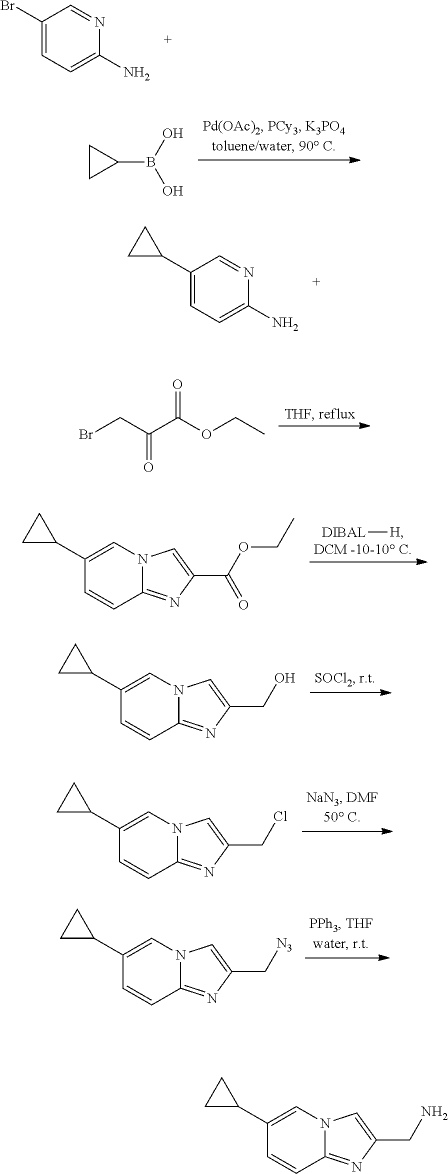

Synthesis of 5-cyclopropylpyridin-2-amine. A mixture of 5-bromopyridin-2-amine (2.0 g, 12 mmol), cyclopropylboronic acid (2.5 g, 29 mmol) and K₃PO₄ (8.6 g, 40 mmol) in toluene (40 mL) and water (2.0 mL) was degassed with N₂ for 10 min. Pd(OAc)₂ (0.26 g, 1.2 mmol) and PCy₃ (0.65 g, 2.3 mmol) were added and the reaction mixture was degassed for a further 5 min then heated to 90° C. and stirred for 18 h. The reaction mixture was cooled to room temperature, diluted with water (60 mL) and extracted with EtOAc (3×50 mL). The combined organic layers were dried over MgSO₄ and concentrated in vacuo. The residue was purified by silica gel chromatography eluting with a gradient of 30-100% EtOAc in isohexane to give the title compound (1.1 g, 71%) as a pale yellow solid. ESI-MS (M+H)⁺: 135. ¹H NMR (400 MHz, CDCl₃) δ7.91 (d, J=1.8 Hz, 1H), 7.12 (dd, J=2.1, 8.5 Hz, 1H), 6.43 (d, J=8.6 Hz, 1H), 4.34-4.19 (m, 2H), 1.82-1.72 (m, 1H), 0.91-0.84 (m, 2H), 0.60-0.54 (m, 2H).

Synthesis of ethyl 6-cyclopropylimidazo[1,2-a]pyridine-2-carboxylate. A mixture of 5-cyclopropylpyridin-2-amine (1.2 g, 8.6 mmol) and ethyl 3-bromo-2-oxopropanoate (1.7 g, 8.6 mmol) in anhydrous THF (50 mL) was stirred at reflux for 18 h. The reaction mixture was cooled to room temperature and the precipitate was filtered, washed with Et₂O and dried in vacuo. The residue was purified by silica gel chromatography eluting with a gradient of 30-100% EtOAc in isohexane to give the title compound (0.93 g, 46%) as a colourless solid. ESI-MS (M+H)⁺: 231, ¹H NMR (400 MHz, CDCl₃) δ8.09 (s, 1H), 7.90 (d, J=0.5 Hz, 1H), 7.57 (d, J=9.4 Hz, 1H), 6.99 (dd, J=1.8, 9.4 Hz, 1H), 4.45 (q, J=7.1 Hz, 2H), 1.94-1.86 (m, 1H), 1.44 (dd, J=7.2, 7.2 Hz, 3H), 1.02-0.97 (m, 2H), 0.73-0.68 (m, 2H).

Synthesis of (6-cyclopropylimidazo[1,2-a]pyridin-2-yl)metanol. A solution of ethyl 6-cyclopropylimidazo[1,2-a]pyridine-2-carboxylate (820 mg, 3.6 mmol) in anhydrous DCM (65 mL) was cooled to −10° C. and a solution of DIBAL-H (1.0 M in THF, 14 mL, 14 mmol) was added dropwise and the reaction mixture was stirred between −10° C. and 10° C. over 30 min. The reaction mixture was quenched with dropwise addition of Rochelle's salt (sat. aq., 25 mL) at 0° C. and diluted with water (30 mL). The mixture was separated, and the aqueous phase was further extracted with DCM (60 mL). The combined organic layers were filtered through Celite®, dried over a hydrophobic frit and concentrated in vacuo to give the title compound (580 mg, 86%) as an off white solid. ESI-MS (M+H)⁺: 189.1, ¹H NMR (400 MHz, DMSO) δ8.34 (d, J=0.5 Hz, 1H), 7.67 (s, 1H), 7.36 (d, J=9.3 Hz, 1H), 6.95 (dd, J=1.8, 9.3 Hz, 1H), 5.13 (dd, J=5.7, 5.7 Hz, 1H), 4.57 (d, J=5.3 Hz, 2H), 1.98-1.90 (m, 1H), 0.96-0.90 (m, 2H), 0.71-0.66 (m, 2H).

Synthesis of 2-(chloromethyl)-6-cyclopropylimidazo[1,2-a]pyridine. A suspension of (6-cyclopropylimidazo[1,2-a]pyridin-2-yl)methanol (250 mg, 1.3 mmol) in thionyl chloride (0.49 mL, 6.7 mmol) was stirred at room temperature for 30 min. The mixture was concentrated in vacuo. The residue was dissolved in DCM (20 mL) and added to warm water (20 mL). The mixture was separated, and the organic layer was washed with NaHCO₃ (sat. aq., 30 mL), dried over a hydrophobic frit and concentrated in vacuo to give the title compound (200 mg, 73%). ¹H NMR (400 MHz, DMSO) δ8.35 (s, 1H), 7.88 (s, 1H), 7.43 (d, J=9.4 Hz, 1H), 7.03 (dd, J=1.8, 9.4 Hz, 1H), 4.83 (s, 2H), 1.99-1.91 (m, 1H), 0.97-0.91 (m, 2H), 0.72-0.67 (m, 2H).

Synthesis of 2-(azidomethyl)-6-cyclopropylimidazo[1,2-a]pyridine. 2-(Chloromethyl)-6-cyclopropylimidazo[1,2-a]pyridine (200 mg, 0.94 mmol) was dissolved in anhydrous DCM (1.0 mL) and sodium azide (120 mg, 1.9 mmol) was added. The reaction mixture was stirred at 50° C. for 1 h. The reaction mixture was cooled to room temperature, diluted with water (5.0 mL) and extracted with DCM (2×10 mL). The combined organic layers were dried over a hydrophobic frit and concentrated in vacuo to give the title compound (190 mg, 92%) as a brown oil. ESI-MS (M+H)⁺: 214.1, ¹H NMR (400 MHz, DMSO) δ8.38 (d, J=0.4 Hz, 1H), 7.86 (s, 1H), 7.45 (d, J=9.4 Hz, 1H), 7.03 (dd, J=1.8, 9.3 Hz, 1H), 4.48 (s, 2H), 2.00-1.92 (m, 1H), 0.97-0.92 (m, 2H), 0.73-0.68 (m, 2H).

Synthesis of (6-cyclopropylimidazo[1,2-a]pyridin-2-yl)methanamine. To a solution of 2-(azidomethyl)-6-cyclopropylimidazo[1,2-a]pyridine (190 mg, 0.80 mmol) in THF (4.0 mL) and water (0.40 mL) was added PPh₃ (420 mg, 1.6 mmol) and the reaction mixture was stirred at room temperature for 16 h. The reaction mixture was concentrated in vacuo. The residue was loaded onto an SCX cartridge, washed with DCM and MeOH and eluted with 3 N NH₃ in MeOH. The eluent was concentrated in vacuo. The residue was further purified by loading onto an SCX cartridge, which was washed with DCM, DCM:MeOH (1:1) and MeOH and eluted with 3 N NH₃ in MeOH. The eluent was concentrated in vacuo to give the title compound (140 mg, 92%) as a yellow oil. ESI-MS (M+H)⁺: 188.2, ¹H NMR (400 MHz, DMSO) δ8.32 (s, 1H), 7.65 (s, 1H), 7.35 (d, J=9.3 Hz, 1H), 6.94 (dd, J=1.8, 9.3 Hz, 1H), 3.78 (d, J=0.6 Hz, 2H), 1.97-1.89 (m, 1H), 0.95-0.90 (m, 2H), 0.71-0.66 (m, 2H). 2 exchangeable protons not visible.

### Synthesis of 6-chloro-N-((6-cyclopropylimidazo[1,2-a]pyridin-2-yl)methyl)pyrimidin-4-amine

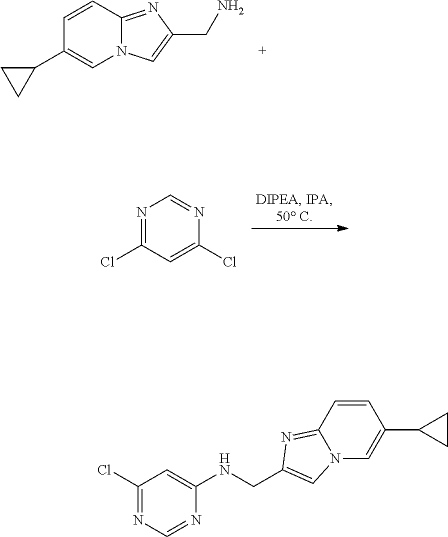 [SMILES: NCc1cn2cc(C3CC3)ccc2n1 ; Clc1cc(Cl)ncn1 ; Clc1cc(NCc2cn3cc(C4CC4)ccc3n2)ncn1]

A mixture of (6-cyclopropylimidazo[1,2-a]pyridin-2-yl)methanamine (970 mg, 5.17 mmol), 4,6-dichloropyrimidine (700 mg, 4.70 mmol), DIPEA (2.5 mL, 14.10 mmol) in iPA (50 mL) was heated to 50° C. for 18 h. Water was added and the solid was collected by filtration to give the title compound (1.38 g, 98%) as a cream solid. ¹H NMR (400 MHz, DMSO) δ8.35 (s, 2H), 8.25 (d, J=4.5 Hz, 1H), 7.73 (s, 1H), 7.47-7.40 (m, 1H), 7.04-7.02 (m, 1H), 6.68 (s, 1H), 4.66-4.66 (m, 2H), 1.97 (dd, J=4.3, 8.3 Hz, 1H), 1.01-0.96 (m, 2H), 0.75-0.69 (m, 2H).

### Synthesis of 4-chloro-N-((6-cyclopropylimidazo[1,2-a]pyridin-2-yl)methyl)pyrimidin-2-amine

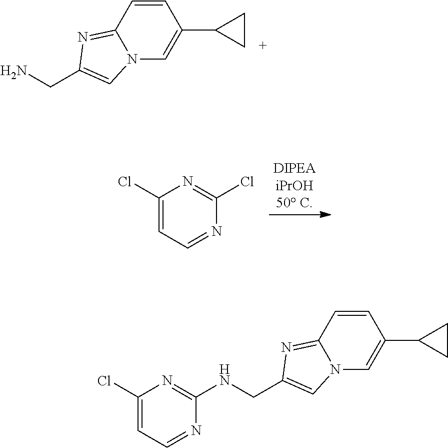 [SMILES: NCc1cn2cc(C3CC3)ccc2n1 ; Clc1ccnc(Cl)n1 ; Clc1ccnc(NCc2cn3cc(C4CC4)ccc3n2)n1]

A mixture of (6-cyclopropylimidazo[1,2-a]pyridin-2-yl)methanamine (200 mg, 1.1 mmol), 2,4-dichloropyrimidine (160 mg, 1.1 mmol) and DIPEA (0.56 mL, 3.2 mmol) in iPrOH (10 mL) was stirred at 50° C. for 16 h. The reaction mixture was cooled to room temperature and concentrated in vacuo. The residue was purified by column chromatography on silica gel, eluting with 0-5% MeOH in DCM to give the title compound (170 mg, 53%) which was used without further purification. ¹H NMR (400 MHz, DMSO) δ8.38-8.30 (m, 2H), 7.98-7.96 (s, 1H), 7.40 (d, J=9.6 Hz, 1H), 7.01-6.97 (m, 1H), 6.56 (d, J=6.0 Hz, 1H), 4.60-4.55 (d, J=5.2 Hz, 2H), 1.97-1.89 (m, 1H), 0.96-0.90 (m, 2H), 0.71-0.65 (m, 2H).

Synthesis of 4-((6-cyclopropylimidazo[1,2-a]pyridin-2-yl)methoxy)pyridin-2-amine

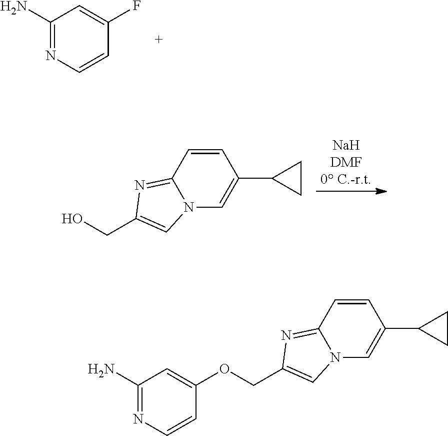 [SMILES: Nc1cc(F)ccn1 ; OCc1cn2cc(C3CC3)ccc2n1 ; Nc1cc(OCc2cn3cc(C4CC4)ccc3n2)ccn1]

NaH (60% in mineral oil, 0.023 g, 0.58 mmol) was added to a solution of (6-cyclopropylimidazo[1,2-a]pyridin-2-yl)methanol (0.10 g, 0.53 mmol) in DMF (5.0 mL) under a N₂ atmosphere. The mixture was stirred at 0° C. for 5 min then at room temperature for 30 min. 4-Fluoropyridin-2-amine (0.060 g, 0.53 mmol) was added at 0° C. and the mixture was stirred at 50° C. for 5 h. The mixture was cooled, diluted with water and extracted with EtOAc. The combined organic layers were dried (MgSO₄), filtered and concentrated under a stream of air. The residue was purified by column chromatography on silica gel, eluting with 0-10% NH₃ in MeOH in DCM to give the title compound (0.065 g, 44%) as a beige solid. ¹H NMR (400 MHz, DMSO) δ8.39 (s, 1H), 7.89 (s, 1H), 7.77 (d, J=5.8 Hz, 1H), 7.48 (d, J=9.3 Hz, 1H), 7.06 (dd, J=1.8, 9.3 Hz, 1H), 6.26 (dd, J=2.3, 6.1 Hz, 1H), 6.11 (d, J=2.3 Hz, 1H), 5.82 (s, 2H), 5.17 (s, 2H), 2.03-1.95 (m, 1H), 1.01-0.94 (m, 2H), 0.76-0.71 (m, 2H).

### Synthesis of 6-((6-cyclopropylimidazo[1,2-a]pyridin-2-yl)methoxy)pyrimidin-4-amine

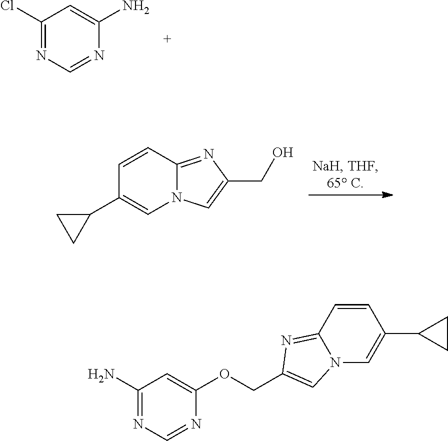 [SMILES: Nc1cc(Cl)ncn1 ; OCc1cn2cc(C3CC3)ccc2n1 ; Nc1cc(OCc2cn3cc(C4CC4)ccc3n2)ncn1]

Sodium hydride (60% in mineral oil, 43 mg, 1.06 mmol) was added to a solution of (6-cyclopropylimidazo[1,2-a]pyridin-2-yl)methanol (100 mg, 0.531 mmol) in THF (10 ml) and stirred at room temperature for 2 h. A solution of 6-chloropyrimidin-4-amine (83 mg, 0.638 mmol) in THF (2 mL) was added and the mixture was heated to 65° C. for 18 h. The mixture was diluted with water (10 mL) and extracted with EtOAc (100 mL). The organic phase was dried (MgSO₄) and concentrated in vacuo. Purification by column chromatography on silica gel, eluting with a gradient of 0-10% MeOH in DCM gave the title compound (77 mg, 52%). ESI-MS (M+H)⁺: 282, ¹H NMR (400 MHz, CDCl₃) δ 8.31 (s, 1H), 7.86 (s, 1H), 7.55 (s, 1H), 7.47 (d, J=9.3 Hz, 1H), 6.96-6.91 (m, 1H), 5.85 (s, 1H), 5.53-5.51 (m, 2H), 4.73-4.68 (m, 2H), 1.92-1.83 (m, 1H), 0.99-0.92 (m, 2H), 0.70-0.64 (m, 2H).

### Synthesis of 6-((6-cyclopropylimidazo[1,2-a]pyridin-2-yl)methoxy)pyridazin-4-amine

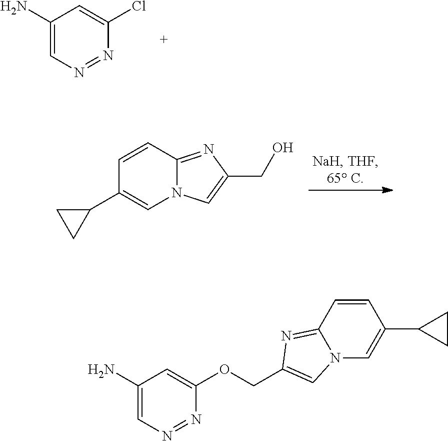 [SMILES: Nc1cnnc(Cl)c1 ; OCc1cn2cc(C3CC3)ccc2n1 ; Nc1cnnc(OCc2cn3cc(C4CC4)ccc3n2)c1]

The title compound (110 mg, 25%) was prepared using a similar procedure to that used for 6-((6-cyclopropylimidazo[1,2-a]pyridin-2-yl)methoxy)pyrimidin-4-amine using (6-cyclopropylimidazo[1,2-a]pyridin-2-yl)methanol and 6-chloropyridazin-4-amine as coupling partners. ESI-MS (M+H)⁺: 282, ¹H NMR (400 MHz, CD₃OD) δ 8.28 (d, J=2.3 Hz, 1H), 8.24-8.22 (m, 1H), 7.83 (d, J=0.6 Hz, 1H), 7.44 (d, J=9.3 Hz, 1H), 7.13 (dd, J=1.7, 9.3 Hz, 1H), 6.19 (d, J=2.3 Hz, 1H), 5.50 (d, J=0.4 Hz, 2H), 2.02-1.94 (m, 1H), 1.04-0.98 (m, 2H), 0.78-0.73 (m, 2H). 2 exchangeable protons not observed.

### Synthesis of N⁴-((6-cyclopropylimidazo[1,2-a]pyridin-2-yl)methyl)pyridine-2,4-diamine

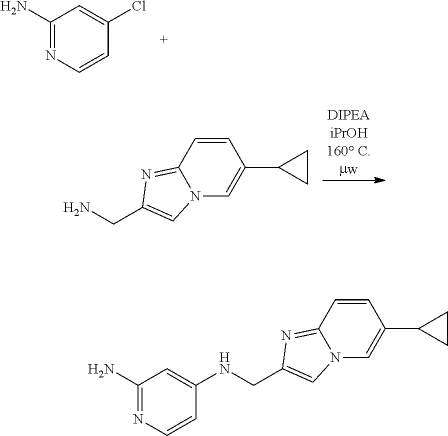 [SMILES: Nc1cc(Cl)ccn1 ; NCc1cn2cc(C3CC3)ccc2n1 ; Nc1cc(NCc2cn3cc(C4CC4)ccc3n2)ccn1]

A mixture of (6-cyclopropylimidazo[1,2-a]pyridin-2-yl)methanamine (0.10 g, 0.53 mmol), 4-chloropyridin-2-amine (0.055 g, 0.43 mmol) and DIPEA (0.14 g, 0.80 mmol) in iPrOH (1.0 mL) was stirred at 160° C. in a microwave for 1 h. The mixture was concentrated in vacuo and the residue was purified by column chromatography on silica gel, eluting with 1-20% NH₃ in MeOH in DCM to give the title compound (0.093 g, 41%) as a yellow gum. ESI-MS (M+H)⁺: 280.2, ¹H NMR (400 MHz, DMSO) δ8.23 (s, 1H), 7.54 (s, 1H), 7.37 (d, J=6.1 Hz, 1H), 7.30 (d, J=9.3 Hz, 1H), 6.89 (dd, J=1.8, 9.3 Hz, 1H), 6.68 (t, J=5.7 Hz, 1H), 5.88 (dd, J=2.0, 6.1 Hz, 1H), 5.56-5.54 (m, 1H), 5.42 (s, 2H), 4.22 (d, J=5.9 Hz, 2H), 1.87-1.79 (m, 1H), 0.86-0.80 (m, 2H), 0.61-0.56 (m, 2H).

### Synthesis of 2-(3-bromo-2-oxopropyl)isoindoline-1,3-dione

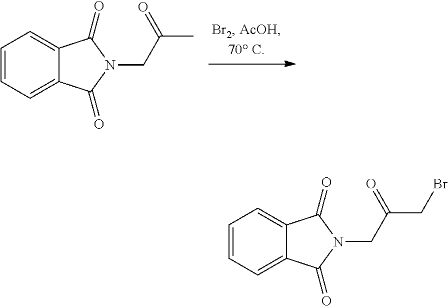 [SMILES: CC(=O)CN1C(=O)c2ccccc2C1=O ; O=C(CBr)CN1C(=O)c2ccccc2C1=O]

A few drops of a solution of bromine (7.6 mL, 150 mmol) in AcOH (80 mL) was added to a stirred solution of 2-(2-oxopropyl)isoindoline-1,3-dione (20 g, 98 mmol) at 70° C. The reaction was stirred until the solution went colourless. The remainder of the bromine/AcOH solution was then added dropwise over 2 h. The reaction mixture was stirred at 70° C. for a further 2 h. The mixture was cooled to room temperature and concentrated in vacuo. The residue was dissolved in DCM (300 mL) and washed with Na₂S₂O₃ solution (1.0 M, 75 mL) and Na₂CO₃ solution (10% aq., 2×150 mL). The organic phase was dried over MgSO₄ and concentrated in vacuo. The residue was triturated with hot diethyl ether, filtered and dried to give the title compound (22.6 g, 81%) as a white solid. ¹H NMR (400 MHz, CDCl₃) δ7.92-7.87 (m, 2H), 7.79-7.73 (m, 2H), 4.78 (s, 2H), 4.01 (s, 2H).

### Synthesis of 1-(2-(aminomethyl)-6-cyclopropylimidazo[1,2-a]pyridin-8-yl)pyrrolidin-2-one

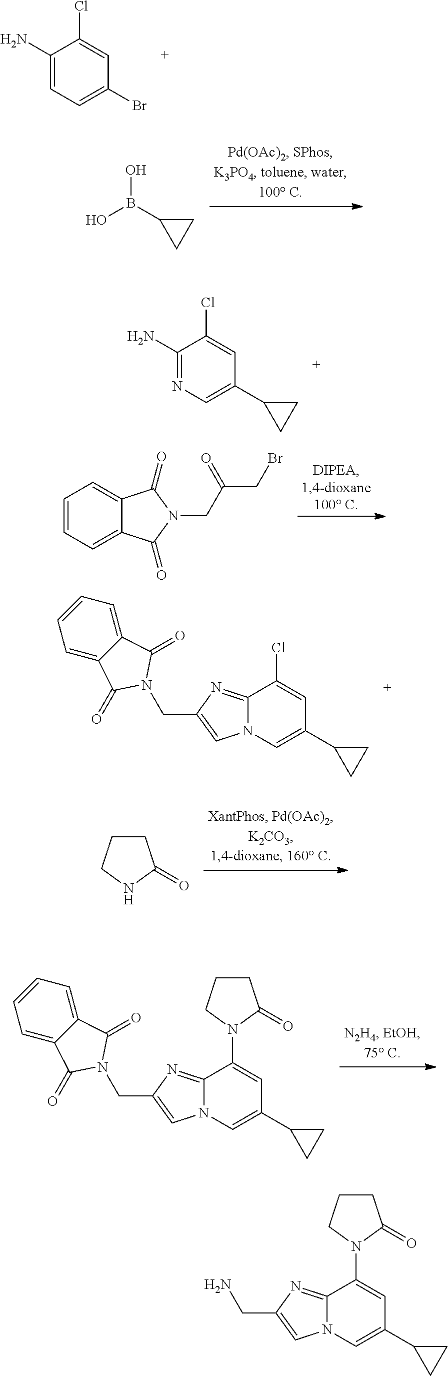

Synthesis of 3-chloro-5-cyclopropylpyridin-2-amine. A mixture of toluene (120 mL) and water (12 mL) were degassed for 50 min. 5-Bromo-3-chloropyridin-2-amine (5.0 g, 24 mmol), cyclopropylboronic acid (2.1 g, 24 mmol), SPhos (0.99 g, 2.4 mmol) and K₃PO₄ (18 g, 84 mmol) were added to the solvent mixture, and it was heated to 100° C. Pd(OAc)₂ (0.27 g, 1.2 mmol) was added and the reaction mixture was stirred at this temperature for 3 h. The mixture was cooled to room temperature and filtered through Celite®, which was washed with toluene. The combined organic phase was dried over MgSO₄ and concentrated in vacuo. The residue was purified by column chromatography on silica gel, eluting with a gradient of 5-30% EtOAc in cyclohexane to give the title compound (3.5 g, 85%) as an orange solid. ESI-MS (M+H)⁺: 169.0, ¹H NMR (400 MHz, CDCl₃) δ7.45 (s, 1H), 6.99 (d, J=1.5 Hz, 1H), 5.10 (s, 2H), 1.88-1.80 (m, 1H), 0.98-0.86 (m, 2H), 0.66-0.61 (m, 2H).

Synthesis of 2-((8-chloro-6-cyclopropylimidazo[1,2-a]pyridin-2-yl)methyl)isoindoline-1,3-dione. A solution of 2-(3-bromo-2-oxopropyl)isoindoline-1,3-dione (890 mg, 3.1 mmol), 3-chloro-5-cyclopropylpyridin-2-amine (530 mg, 3.1 mmol) in 1,4-dioxane (11 mL) was heated at 98° C. for 17 h. The mixture was cooled to room temperature and concentrated in vacuo. The mixture was partitioned between DCM (2×50 mL) and NaHCO₃ (sat. aq., 50 mL). The combined organic layers were dried over MgSO₄ and concentrated in vacuo. The residue was purified by column chromatography on silica gel, eluting with a gradient of 10-50% EtOAc in isohexane to give the title compound (880 mg, 84%). ESI-MS (M+H)⁺: 352.1, ¹H NMR (400 MHz, CDCl₃) δ7.91-7.85 (m, 2H), 7.76-7.70 (m, 3H), 7.46 (s, 1H), 6.99 (s, 1H), 5.10 (s, 2H), 1.87-1.80 (m, 1H), 0.99-0.89 (m, 2H), 0.67-0.60 (m, 2H).

Synthesis of 2-((6-cyclopropyl-8-(2-oxopyrrolidin-1-yl)imidazo[1,2-a]pyridin-2-yl)methyl)isoindoline-1,3-dione. Pyrrolidine-2-one (0.65 mL, 8.5 mmol), 2-((8-chloro-6-cyclopropylimidazo[1,2-a]pyridin-2-yl)methyl)isoindoline-1,3-dione (2000 mg, 5.7 mmol), Xantphos (660 mg, 1.1 mmol) and K₂CO₃ (1600 mg, 11 mmol) in 1,4-dioxane (20 mL) was degassed for 5 min with N₂. Pd(OAc)₂ (130 mg, 0.57 mmol) was added and the reaction mixture was heated in a microwave at 160° C. for 2 h. The mixture was cooled to room temperature, filtered through Celite® and concentrated in vacuo. The residue was purified by silica gel chromatography eluting with a gradient of 20-100% EtOAc in cyclohexane to give the title compound (1.0 g, 44%). ESI-MS (M+H)⁺: 401.3, ¹H NMR (400 MHz, CDCl₃) δ7.87 (dd, J=3.0, 5.5 Hz, 2H), 7.72 (dd, J=3.1, 5.5 Hz, 2H), 7.69-7.68 (m, 1H), 7.45 (s, 1H), 7.24 (d, J=1.5 Hz, 1H), 5.02 (s, 2H), 4.27 (dd, J=7.1, 7.1 Hz, 2H), 2.58 (t, J=8.2 Hz, 2H), 2.22-2.13 (m, 2H), 1.90-1.82 (m, 1H), 0.95-0.89 (m, 2H), 0.68-0.62 (m, 2H).

Synthesis of 1-(2-(aminomethyl)-6-cyclopropylimidazo[1,2-a]pyridin-8-yl)pyrrolidin-2-one. A mixture of 2-((6-cyclopropyl-8-(2-oxopyrrolidin-1-yl)imidazo[1,2-a]pyridin-2-yl)methyl)isoindoline-1,3-dione (1000 mg, 2.5 mmol) and hydrazine hydrate (0.16 mL, 5.0 mmol) in EtOH (15 mL) was stirred at 75° C. for 1 h. The mixture was cooled to room temperature, and loaded onto an SCX cartridge, which was washed with 25% MeOH in DCM, and eluted with 25% 7 N NH₃ in MeOH in DCM. The eluent was concentrated in vacuo to give the title compound (600 mg, 89%). ESI-MS (M+H)⁺: 271.3, ¹H NMR (400 MHz, CDCl₃) δ7.77 (s, 1H), 7.40 (s, 1H), 7.19 (d, J=1.5 Hz, 1H), 4.28 (t, J=7.2 Hz, 2H), 3.99 (d, J=0.6 Hz, 2H), 2.62 (t, J=8.2 Hz, 2H), 2.28-2.20 (m, 2H), 1.93-1.85 (m, 1H), 0.97-0.91 (m, 2H), 0.72-0.66 (m, 2H). NH₂ not observed

### Synthesis of 1-(2-(((6-chloropyrimidin-4-yl)amino)methyl)-6-cyclopropylimidazo[1,2-a]pyridin-8-yl)pyrrolidin-2-one

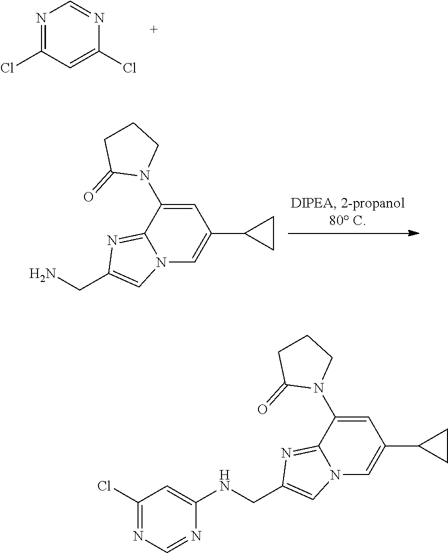 [SMILES: Clc1cc(Cl)ncn1 ; NCc1cn2cc(C3CC3)cc(N3CCCC3=O)c2n1 ; O=C1CCCN1c1cc(C2CC2)cn2cc(CNc3cc(Cl)ncn3)nc12]

A solution of 4,6-dichloropyrimidine (0.12 g, 0.84 mmol), 1-(2-(aminomethyl)-6-cyclopropylimidazo[1,2-a]pyridin-8-yl)pyrrolidin-2-one (0.25 g, 0.92 mmol) and DIPEA (0.44 mL, 2.52 mmol) in 2-propanol (5 mL) was heated to 70° C. for 1 h. The mixture was cooled to room temperature, diluted with water (20 mL) then extracted with DCM (3×10 mL). The combined organics were dried over MgSO₄ then concentrated in vacuo. The residue was triturated with diethyl ether to give the crude product which was used without further purification (0.27 g, 84%). ¹H NMR (400 MHz, DMSO) δ8.35 (s, 1H), 8.30 (d, J=1.0 Hz, 1H), 8.25 (s, 1H), 7.77 (s, 1H), 7.18 (s, 1H), 6.69 (s, 1H), 4.67-4.66 (m, 2H), 4.19 (dd, J=7.1, 7.1 Hz, 2H), 2.02-1.93 (m, 1H), 1.32 (dd, J=7.2, 7.2 Hz, 4H), 0.98 (ddd, J=4.4, 6.4, 8.4 Hz, 2H), 0.72-0.67 (m, 2H).

### Synthesis of 1-(2-(((6-chloropyrimidin-4-yl)(methyl)amino)methyl)-6-cyclopropylimidazo[1,2-a]pyridin-8-yl)pyrrolidin-2-one

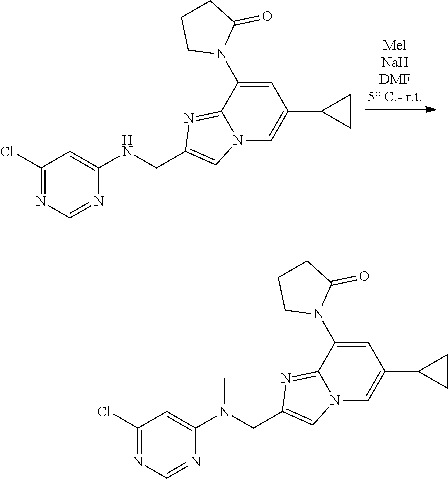 [SMILES: O=C1CCCN1c1cc(C2CC2)cn2cc(CNc3cc(Cl)ncn3)nc12 ; CN(Cc1cn2cc(C3CC3)cc(N3CCCC3=O)c2n1)c1cc(Cl)ncn1]

NaH (60% in mineral oil, 0.0048 g, 0.12 mmol) was added to a stirred solution of 1-(2-(((6-chloropyrimidin-4-yl)amino)methyl)-6-cyclopropylimidazo[1,2-a]pyridin-8-yl)pyrrolidin-2-one (0.031 g, 0.080 mmol) in DMF (1.0 mL). The mixture was stirred at room temperature for 30 min then cooled to 5° C. and a solution of MeI (0.16 M in DMF, 0.1 mL, 0.080 mmol) was added. The mixture was stirred at 0° C. for 30 min then warmed to room temperature and stirred for 2 h. Further MeI (0.16 M in DMF, 0.1 mL, 0.080 mmol) was added and the mixture was stirred at room temperature for 18 h. The mixture was diluted with water (10 mL) and extracted with EtOAc (3×2.0 mL). The combined organics were washed with brine (sat. aq.), dried (MgSO₄), filtered and concentrated in vacuo to give the title compound (0.026 g, 81%) as a brown sticky residue. ESI-MS (M+H)⁺: 397.2, 399.2.

### Synthesis of 1-(2-(((2-bromopyridin-4-yl)amino)methyl)-6-cyclopropylimidazo[1,2-a]pyridin-8-yl)pyrrolidin-2-one

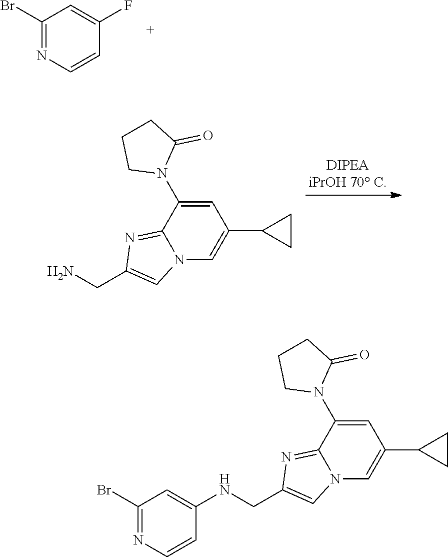 [SMILES: Fc1ccnc(Br)c1 ; NCc1cn2cc(C3CC3)cc(N3CCCC3=O)c2n1 ; O=C1CCCN1c1cc(C2CC2)cn2cc(CNc3ccnc(Br)c3)nc12]

A mixture of 2-bromo-4-fluoropyridine (0.057 mL, 0.56 mmol), 1-(2-(aminomethyl)-6-cyclopropylimidazo[1,2-a]pyridin-8-yl)pyrrolidin-2-one (0.15 g, 0.56 mmol) and DIPEA (0.29 mL, 1.7 mmol) in iPrOH (1.0 mL) was stirred at 70° C. for 18 h. The mixture was cooled and concentrated in vacuo. The residue was purified by column chromatography on silica gel, eluting with 1-20% NH₃ MeOH in DCM to give the title compound (0.12 g, 52%) as a yellow gum. ¹H NMR (400 MHz, DMSO) δ8.27 (d, J=1.1 Hz, 1H), 7.79 (d, J=5.8 Hz, 1H), 7.74 (s, 1H), 7.43 (t, J=5.8 Hz, 1H), 7.16-7.14 (m, 1H), 6.82 (d, J=2.1 Hz, 1H), 6.62 (dd, J=2.1, 5.8 Hz, 1H), 4.40 (d, J=5.8 Hz, 2H), 4.16 (t, J=7.1 Hz, 2H), 2.51-2.46 (m, 2H), 2.18-2.09 (m, 2H), 2.01-1.90 (m, 1H), 0.99-0.91 (m, 2H), 0.69-0.64 (m, 2H).

### Synthesis of 2-(aminomethyl)-6-cyclopropylimidazo[1,2-a]pyridine-8-carbonitrile hydrochloride

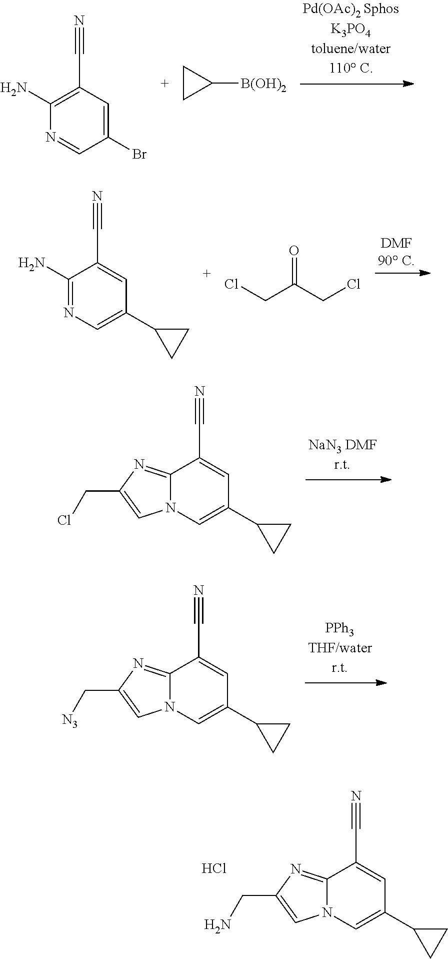

Synthesis of 2-amino-5-cyclopropylnicotinonitrile. A mixture of 2-amino-5-bromonicotinonitrile (2.0 g, 10 mmol), cyclopropylboronic acid (2.2 g, 25 mmol), SPhos (410 mg, 1.0 mmol) and K₃PO₄ (7.5 g, 35 mmol) in toluene (48 mL) and water (4.8 mL) was degassed with nitrogen. Pd(OAc)₂ (110 mg, 0.5 mmol) was added and the mixture was stirred at reflux for 6 h under a nitrogen atmosphere. The reaction was cooled and filtered with toluene. The filtrate was washed with water, NaOH (2.0M aq.×2) and brine (sat. aq.), dried over MgSO₄, filtered and concentrated in vacuo. The residue was purified by column chromatography on silica gel, eluting with a gradient of 10-70% EtOAc in isohexane to give the title compound (1.2 g, 75%) as a yellow solid. ¹H NMR (400 MHz, CDCl₃) δ 8.10 (d, J=2.4 Hz, 1H), 7.37 (d, J=2.4 Hz, 1H), 5.02 (s, 2H), 1.83-1.76 (m, 1H), 0.97-0.92 (m, 2H), 0.62-0.57 (m, 2H).

Synthesis of 2-(chloromethyl)-6-cyclopropylimidazo[1,2-a]pyridine-8-carbonitrile. A mixture of 2-amino-5-cyclopropylnicotinonitrile (1.1 g, 6.9 mmol) and 1,3-dichloroacetone (1.1 g, 9.0 mmol) were combined in DMF (50 mL) and stirred at 95° C. for 18 h. The mixture was cooled, diluted with Et₂O (250 mL) and filtered. The mixture was washed with water (3×100 mL), dried over MgSO₄, filtered and concentrated in vacuo. The residue was purified by column chromatography on silica gel, eluting with a gradient of 10-90% EtOAc in isohexane to give the title compound (630 mg 39%) as a purple solid. ¹H NMR (400 MHz, DMSO) δ8.69 (d, J=1.1 Hz, 1H), 8.05 (s, 1H), 7.80 (d, J=1.6 Hz, 1H), 4.88 (s, 2H), 2.03-1.95 (m, 1H), 0.99-0.94 (m, 2H), 0.80-0.75 (m, 2H).

Synthesis of 2-(azidomethyl)-6-cyclopropylimidazo[1,2-a]pyridine-8-carbonitrile. 2-(Chloromethyl)-6-cyclopropylimidazo[1,2-a]pyridine-8-carbonitrile (630 mg, 2.70 mmol) and NaN₃ (230 mg, 3.5 mmol) were combined in DMF (5.0 mL) and stirred at room temperature under a nitrogen atmosphere for 18 h. The mixture was diluted with EtOAc (50 mL) and washed with water (2×50 mL) and brine (50 mL), then dried over MgSO₄, filtered and concentrated in vacuo to give the title compound (580 mg, 89%) as a purple oil. ESI-MS (M+H)⁺: 239.2, ¹H NMR (400 MHz, DMSO) δ8.72 (dd, J=0.5, 1.7 Hz, 1H), 8.02 (s, 1H), 7.80 (d, J=1.8 Hz, 1H), 4.56 (s, 2H), 2.03-1.96 (m, 1H), 0.99-0.94 (m, 2H), 0.80-0.75 (m, 2H).

Synthesis of 2-(aminomethyl)-6-cyclopropylimidazo[1,2-a]pyridine-8-carbonitrile hydrochloride. 2-(Azidomethyl)-6-cyclopropylimidazo[1,2-a]pyridine-8-carbonitrile (580 mg, 2.4 mmol) and triphenyl phosphine (1.3 g, 4.8 mmol) were combined in THF (11 mL) and water (1.0 mL) and stirred at room temperature for 24 h. The mixture was then concentrated in vacuo, dissolved in DCM and treated with HCl in dioxane (4.0 M). The precipitate was collected by filtration and washed with DCM to give the title compound (630 mg, quant.) as a pale yellow solid. ESI-MS (M+H)⁺: 213.2, ¹H NMR (400 MHz, DMSO) δ8.81 (s, 1H), 8.38 (br s, 3H), 8.06 (s, 1H), 7.85 (s, 1H), 4.19 (d, J=5.6 Hz, 2H), 2.04-1.97 (m, 1H), 1.02-0.95 (m, 2H), 0.82-0.76 (m, 2H).

### Synthesis of 2-(((6-chloropyrimidin-4-yl)amino)methyl)-6-cyclopropylimidazo[1,2-a]pyridine-8-carbonitrile

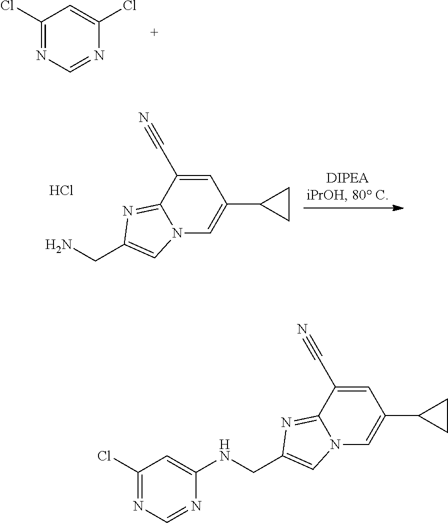 [SMILES: Clc1cc(Cl)ncn1 ; Cl ; N#Cc1cc(C2CC2)cn2cc(CN)nc12 ; N#Cc1cc(C2CC2)cn2cc(CNc3cc(Cl)ncn3)nc12]

DIPEA (0.38 mL, 2.19 mmol) was added to a stirred mixture of 4,6-dichloropyrimidine (160 mg, 1.05 mmol) and 2-(aminomethyl)-6-cyclopropylimidazo[1,2-a]pyridine-8-carbonitrile hydrochloride (250 mg, 0.877 mmol) in iPrOH (7.0 mL). The reaction was then heated to 80° C. for 2.5 h, the reaction was then cooled to room temperature and poured into water. The resulting precipitate was collected by vacuum filtration and washed with water to give the title compound (210 mg, 75%) as a white solid. ESI-MS (M+H)⁺: 325.1, ¹H NMR (400 MHz, CDCl₃) δ8.40 (s, 1H), 8.07-8.06 (m, 1H), 7.56 (s, 1H), 7.42 (d, J=1.6 Hz, 1H), 6.44 (s, 1H), 5.91 (s, 1H), 4.78-4.72 (m, 2H), 1.97-1.89 (m, 1H), 1.05 (ddd, J=3.6, 4.8, 9.9 Hz, 2H), 0.73-0.68 (m, 2H).

### Synthesis of 6-cyclopropyl-2-(hydroxymethyl)imidazo[1,2-a]pyridine-8-carbonitrile

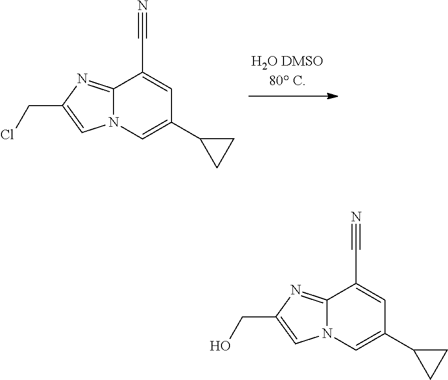 [SMILES: N#Cc1cc(C2CC2)cn2cc(CCl)nc12 ; N#Cc1cc(C2CC2)cn2cc(CO)nc12]

A solution of 2-(chloromethyl)-6-cyclopropylimidazo[1,2-a]pyridine-8-carbonitrile (710 mg, 2.4 mmol) in water (6.0 mL) and DMSO (6.0 mL) was heated to 80° C. and stirred for 16 h. The mixture was allowed to cool to room temperature, loaded onto an SCX cartridge, washed with MeOH and 50% MeOH in DCM and eluted with 7 N NH₃ in MeOH. The eluent was concentrated in vacuo. The residue was purified by column chromatography on silica gel, eluting with a gradient of 0-10% MeOH in DCM to give two fractions. The impure fraction was triturated with DCM, combined with the previously isolated fraction and concentrated in vacuo to give the title compound (310 mg, 59%). ESI-MS (M+H)⁺: 214.2

### Synthesis of 2-(((6-chloropyrimidin-4-yl)oxy)methyl)-6-cyclopropylimidazo[1,2-a]pyridine-8-carbonitrile

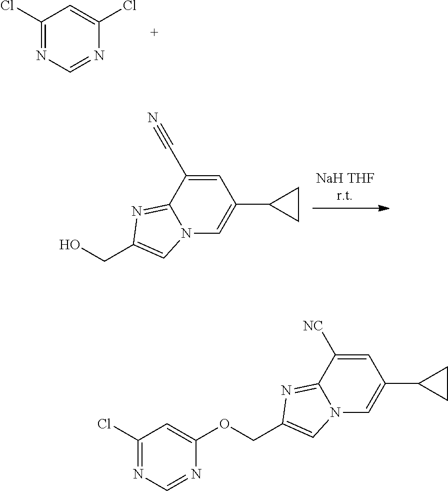 [SMILES: Clc1cc(Cl)ncn1 ; N#Cc1cc(C2CC2)cn2cc(CO)nc12 ; N#Cc1cc(C2CC2)cn2cc(COc3cc(Cl)ncn3)nc12]

6-Cyclopropyl-2-(hydroxymethyl)imidazo[1,2-a]pyridine-8-carbonitrile (0.10 g, 0.47 mmol) was added to a stirred suspension of NaH (60% in mineral oil, 0.021 g, 0.52 mmol) in THF (10 mL) and the mixture was stirred at room temperature for 30 min. A solution of 4,6-dichloropyrimidine (0.084 g, 0.57 mmol) in THF (2.0 mL) was added dropwise and the mixture was stirred at room temperature for 3.5 h. Water was added and the mixture was extracted with DCM (×3), passed through a phase separator cartridge and concentrated in vacuo. The residue was triturated with Et₂O, filtered and dried in vacuo to give the title compound (0.12 g, 76%) as a beige solid. ¹H NMR (400 MHz, DMSO) δ8.86-8.79 (m, 2H), 8.20-8.17 (m, 1H), 7.93-7.90 (m, 1H), 7.39 (s, 1H), 5.71-5.68 (m, 2H), 2.14-2.06 (m, 1H), 1.11-1.04 (m, 2H), 0.89-0.85 (m, 2H).

### Synthesis of 2-(((2-aminopyridin-4-yl)amino)methyl)-6-cyclopropylimidazo[1,2-a]pyridine-8-carbonitrile

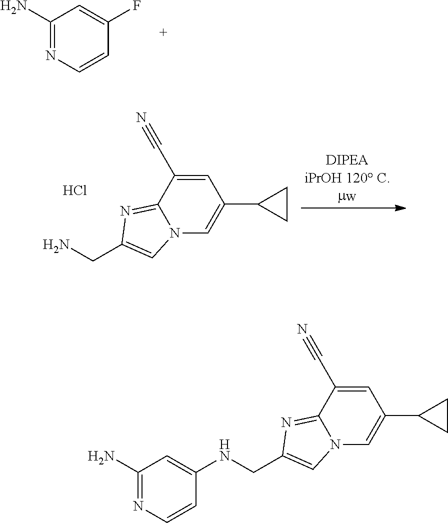 [SMILES: Nc1cc(F)ccn1 ; N#Cc1cc(C2CC2)cn2cc(CN)nc12 ; Cl ; N#Cc1cc(C2CC2)cn2cc(CNc3ccnc(N)c3)nc12]

A mixture of 2-(aminomethyl)-6-cyclopropylimidazo[1,2-a]pyridine-8-carbonitrile hydrochloride (0.075 g, 0.26 mmol), 4-fluoropyridin-2-amine (0.020 mL, 0.26 mmol) and DIPEA (0.18 mL, 1.1 mmol) in iPrOH (5.0 mL) was degassed with N₂ and sealed. The mixture was heated at 120° C. in a microwave for 30 min then for a further 1 h. The mixture was heated at 140° C. in a microwave for 1 h then at 150° for 6 h. Water was added and the mixture was extracted with EtOAc. The combined organic layers were passed through a hydrophobic frit and concentrated in vacuo. The residue was purified by column chromatography on silica gel, eluting with 0-20% NH₃ in MeOH in DCM to give the title compound (0.042 g, 53%). ESI-MS (M+H)⁺: 305.2.

### Synthesis of 3-(2-(aminomethyl)-6-cyclopropylimidazo[1,2-a]pyridin-8-yl)oxetan-3-ol

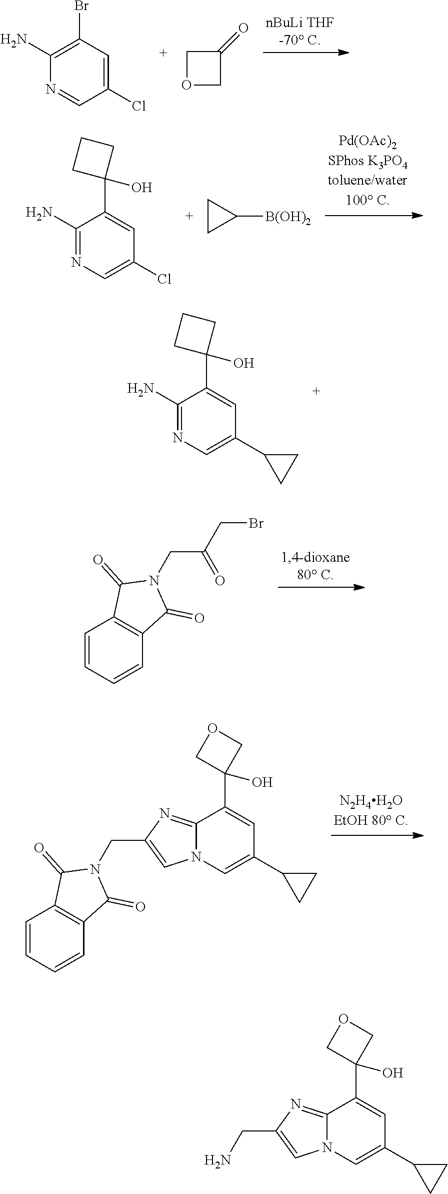

Synthesis of 3-(2-amino-5-chloropyridin-3-yl)oxetan-3-ol. n-BuLi (2.4 M in hexanes, 9.2 mL, 22 mmol) was added dropwise to a stirred solution of 3-bromo-5-chloropyridin-2-amine (1.1 g, 5.5 mmol) in anhydrous THF (40 mL) at −70° C. under a nitrogen atmosphere. The mixture was stirred at −70° C. for 1 h then a solution of oxetan-3-one (1.8 mL, 22 mmol) in THF (10 mL) was added slowly over 10 min. The resultant mixture was warmed to room temperature and stirred for 1 h. NH₄Cl (sat. aq., 15 mL) was added and the mixture was extracted with EtOAc (3×25 mL). The combined organic layers were dried over MgSO₄, filtered and concentrated in vacuo. The residue was purified by column chromatography on silica gel, eluting with a gradient of 0-10% MeOH in DCM to give the title compound (460 mg, 42%) as a pale brown solid. ESI-MS (M+H)⁺: 201.1, ¹H NMR (400 MHz, CDCl₃) δ8.01 (d, J=2.4 Hz, 1H), 7.45 (d, J=2.4 Hz, 1H), 5.04-5.00 (m, 4H), 4.90-4.87 (m, 2H), 2.76 (br s, 1H).

Synthesis of 3-(2-amino-5-cyclopropylpyridin-3-yl)oxetan-3-ol. A mixture of 3-(2-amino-5-chloropyridin-3-yl)oxetan-3-ol (550 mg, 2.8 mmol), cyclopropylboronic acid (350 mg, 4.1 mmol), SPhos (110 mg, 0.28 mmol), Pd(OAc)₂ (62 mg, 0.28 mmol) and K₃PO₄ (2.1 g, 9.6 mmol) in toluene (30 mL) and water (3.0 mL) was degassed with nitrogen then stirred at 100° C. for 18 h under a nitrogen atmosphere. The mixture was cooled and filtered through Celite® which was washed with EtOAc (75 mL). The combined filtrate was dried over MgSO₄, filtered and concentrated in vacuo. The residue was purified by column chromatography on silica gel, eluting with a gradient of 0-10% MeOH in DCM to give the title compound (400 mg, 70%) as a pale yellow solid. ESI-MS (M+H)⁺: 207.1, ¹H NMR (400 MHz, CDCl₃) δ7.85 (d, J=1.9 Hz, 1H), 7.16 (d, J=2.3 Hz, 1H), 5.07 (dd, J=0.8, 7.3 Hz, 2H), 4.89 (dd, J=0.8, 7.3 Hz, 2H), 4.75 (br s, 2H), 2.95 (br s, 1H), 1.85-1.78 (m, 1H), 0.94-0.88 (m, 2H), 0.62-0.57 (m, 2H).

Synthesis of 2-((6-cyclopropyl-8-(3-hydroxyoxetan-3-yl)imidazo[1,2-a]pyridin-2-yl)methyl)isoindoline-1,3-dione. A mixture of 3-(2-amino-5-cyclopropylpyridin-3-yl)oxetan-3-ol (210 mg, 1.0 mmol) and 2-(3-bromo-2-oxopropyl)isoindoline-1,3-dione (280 mg, 1.0 mmol) in 1,4-dioxane (4.0 mL) was stirred at 80° C. for 18 h. The mixture was cooled to room temperature and diluted with EtOAc and K₂CO₃ (10% aq.). The mixture was separated, and the aqueous layer was further extracted with EtOAc. The combined organic layers were washed with brine (sat. aq.), dried over MgSO₄, filtered and concentrated in vacuo. The residue was purified by column chromatography on silica gel, eluting with a gradient of 1-6% MeOH in DCM to give the title compound (210 mg, 54%) as a light yellow solid. ESI-MS [M+H]⁺: 390.2, ¹H NMR (400 MHz, CDCl₃) δ7.88-7.85 (m, 2H), 7.76-7.75 (m, 1H), 7.74-7.71 (m, 2H), 7.47 (s, 1H), 7.36 (s, 1H), 7.24 (d, J=1.6 Hz, 1H), 5.02-4.99 (m, 4H), 4.77 (d, J=7.3 Hz, 2H), 2.01-1.88 (m, 1H), 1.01-0.95 (m, 2H), 0.70-0.65 (m, 2H).

Synthesis of 3-(2-(aminomethyl)-6-cyclopropylimidazo[1,2-a]pyridin-8-yl)oxetan-3-ol. A mixture of 2-((6-cyclopropyl-8-(3-hydroxyoxetan-3-yl)imidazo[1,2-a]pyridin-2-yl)methyl)isoindoline-1,3-dione (120 mg, 0.31 mmol) and hydrazine hydrate (87 μL, 1.8 mmol) in EtOH (3.0 mL) was heated at reflux for 1 h. The mixture was cooled to room temperature, filtered and concentrated in vacuo. The residue was redissolved in MeOH, filtered and concentrated in vacuo. The residue was purified by column chromatography on reverse phase silica gel, eluting with a gradient of 30% MeCN in water (0.1% ((NH₄)₂CO₃) to give the title compound (0.029 g, 36%) as an off-white solid. ¹H NMR (400 MHz, CD₃OD) δ 8.19 (s, 1H), 7.84 (s, 1H), 7.21 (s, 1H), 5.37 (d, J=6.8 Hz, 2H), 4.25 (s, 2H), 3.35 (s, 2H), 2.04-1.94 (m, 1H), 1.03-0.96 (m, 2H), 0.76-0.71 (m, 2H). Exchangeable protons not observed.

### Synthesis of 3-(2-(((6-chloropyrimidin-4-yl)amino)methyl)-6-cyclopropylimidazo[1,2-a]pyridin-8-yl)oxetan-3-ol

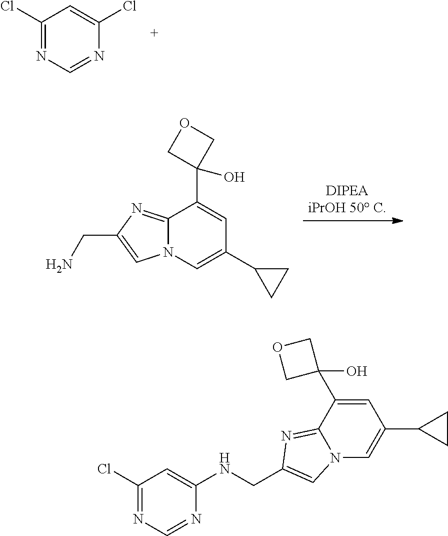 [SMILES: Clc1cc(Cl)ncn1 ; NCc1cn2cc(C3CC3)cc(C3(O)COC3)c2n1 ; OC1(c2cc(C3CC3)cn3cc(CNc4cc(Cl)ncn4)nc23)COC1]

A mixture of 3-(2-(aminomethyl)-6-cyclopropylimidazo[1,2-a]pyridin-8-yl)oxetan-3-ol (0.25 g, 1.3 mmol) and 4,6-dichloropyrimidine (0.18 g, 1.2 mmol) and DIPEA (0.63 mL, 3.6 mmol) in iPrOH (10 mL) was stirred at 50° C. for 3 h. The mixture was cooled, loaded onto silica and purified by column chromatography on silica gel, eluting with 0-10% MeOH in DCM to give the title compound (0.064 g, 35%). ¹H NMR (400 MHz, DMSO) δ8.35-8.30 (m, 3H), 7.75 (s, 1H), 7.10 (s, 1H), 6.68 (s, 1H), 6.52 (s, 1H), 5.31-5.27 (m, 2H), 4.74-4.68 (m, 4H), 2.05-1.95 (m, 1H), 1.00-0.94 (m, 2H), 0.76-0.71 (m, 2H).

Synthesis of 6-cyclopropyl-8-(3-fluorooxetan-3-yl)imidazo[1,2-a]pyridin-2-yl)methanamine

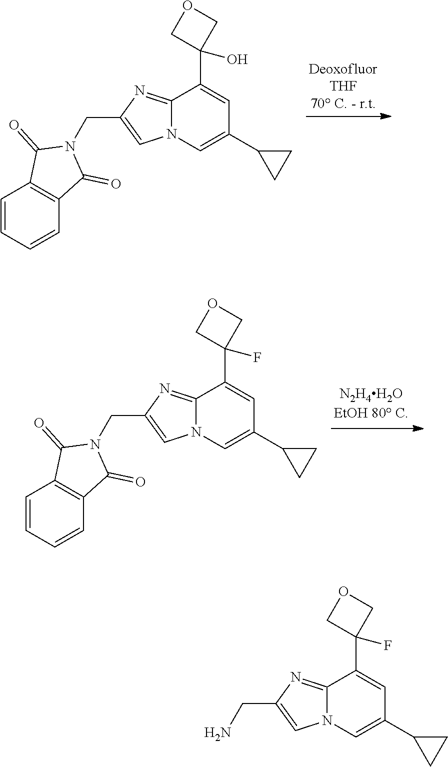 [SMILES: O=C1c2ccccc2C(=O)N1Cc1cn2cc(C3CC3)cc(C3(O)COC3)c2n1 ; O=C1c2ccccc2C(=O)N1Cc1cn2cc(C3CC3)cc(C3(F)COC3)c2n1 ; NCc1cn2cc(C3CC3)cc(C3(F)COC3)c2n1]

Synthesis of 2-((6-cyclopropyl-8-(3-fluorooxetan-3-yl)imidazo[1,2-a]pyridin-2-yl)methyl)isoindoline-1,3-dione. Deoxofluor® (50% in THF, 0.6 mL, 1.6 mmol) was added to a solution of 2-((6-cyclopropyl-8-(3-hydroxyoxetan-3-yl)imidazo[1,2-a]pyridin-2-yl)methyl)isoindoline-1,3-dione (420 mg, 1.1 mmol) in DCM (20 mL) at −70° C. The mixture was warmed to 0° C. and stirred for 3 h. Further Deoxofluor® (50% in THF, 0.3 mL, 0.82 mmol) was added and the mixture was stirred at 0° C. for a further 2 h. The mixture was warmed to room temperature and stirred for 18 h. The mixture was cooled to 0° C. and NaHCO₃ (sat. aq., 15 mL) was added. The mixture was separated, and the organic layer was dried over MgSO₄, filtered and concentrated in vacuo. The residue was purified by column chromatography on silica gel, eluting with a gradient of 20-60% EtOAc in cyclohexane to give the title compound (190 mg, 44%) as a colourless solid. ESI-MS (M+H)⁺: 392.1, ¹H NMR (400 MHz, CDCl₃) δ7.91-7.86 (m, 2H), 7.79 (s, 1H), 7.75-7.70 (m, 2H), 7.43 (s, 1H), 6.95 (s, 1H), 5.53-5.41 (m, 2H), 5.08 (s, 2H), 5.06-4.95 (m, 2H), 1.90-1.82 (m, 1H), 0.98-0.90 (m, 2H), 0.66-0.60 (m, 2H).

Synthesis of (6-cyclopropyl-8-(3-fluorooxetan-3-yl)imidazo[1,2-a]pyridin-2-yl)methanamine. A mixture of 2-((6-cyclopropyl-8-(3-fluorooxetan-3-yl)imidazo[1,2-a]pyridin-2-yl)methyl)isoindoline-1,3-dione (180 mg, 0.46 mmol) in EtOH (5 mL) and hydrazine monohydrate (67 μL, 1.4 mmol) was stirred at reflux for 1 h. The mixture was cooled and concentrated in vacuo. The residue was dissolved in a mixture of DCM/MeOH (9:1, 8 mL), filtered and concentrated in vacuo to give the title compound (110 mg, 95%) as a viscous pale brown oil. ESI-MS (M+H)⁺: 262.0, ¹H NMR (400 MHz, CDCl₃) δ7.88 (s, 1H), 7.44 (s, 1H), 6.97 (s, 1H), 5.56-5.46 (m, 2H), 5.14-5.04 (m, 2H), 4.02 (s, 2H), 1.01-0.93 (m, 2H), 0.70-0.64 (m, 2H). Exchangeable prótons not observed, cyclopropyl CH signal obscured by water signal.

### 6-chloro-N-((6-cyclopropyl-8-(3-fluorooxetan-3-yl)imidazo[1,2-a]pyridin-2-yl)methyl)pyrimidin-4-amine

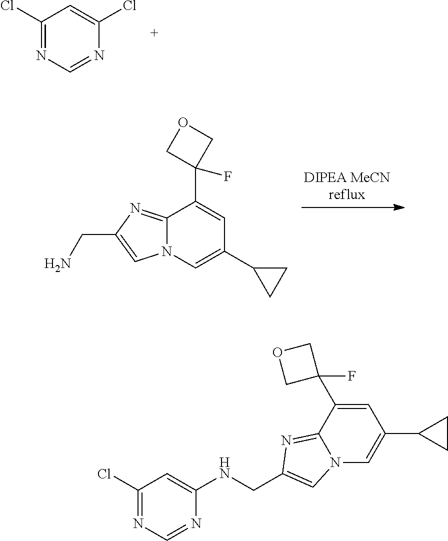 [SMILES: Clc1cc(Cl)ncn1 ; NCc1cn2cc(C3CC3)cc(C3(F)COC3)c2n1 ; FC1(c2cc(C3CC3)cn3cc(CNc4cc(Cl)ncn4)nc23)COC1]

A mixture of (6-cyclopropyl-8-(3-fluorooxetan-3-yl)imidazo[1,2-a]pyridin-2-yl)methanamine (0.73 g, 0.28 mmol), 4,6-dichloropyrimidine (0.042 g, 0.28 mmol) and DIPEA (0.15 mL, 0.84 mmol) in MeCN (2.0 mL) was stirred at reflux for 6 h. The mixture was cooled and concentrated in vacuo. The residue was purified by column chromatography on silica gel, eluting with 1-6% MeOH in DCM to give the title compound (0.071 g, 67%) as a colourless oil. ¹H NMR (400 MHz, CDCl₃) δ8.39 (s, 1H), 7.88 (s, 1H), 7.49-7.46 (m, 1H), 7.04-7.01 (m, 1H), 6.50-6.47 (m, 1H), 5.88 (s, 1H), 5.49 (dd, J=8.0, 26.1 Hz, 2H), 5.10 (dd, J=8.0, 23.1 Hz, 2H), 4.68-4.68 (m, 2H), 1.95-1.86 (m, 1H), 1.02-0.96 (m, 2H), 0.71-0.65 (m, 2H).

### Synthesis of (6-cyclopropyl-8-(4-methylpiperazin-1-yl)imidazo[1,2-a]pyridin-2-yl)methanamine

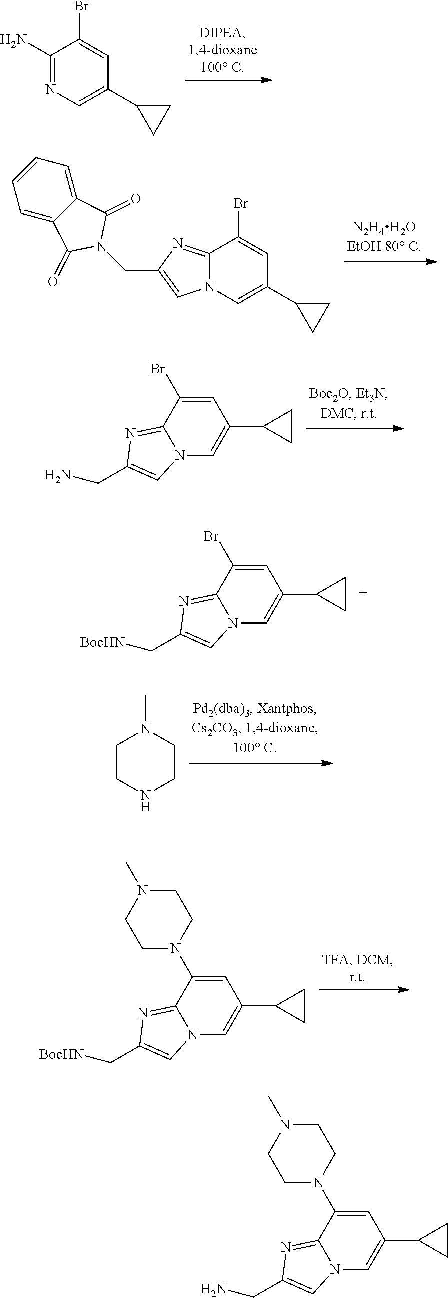

Synthesis of 2-((8-bromo-6-cyclopropylimidazo[1,2-a]pyridin-2-yl)methyl)isoindoline-1,3-dione. A solution of 2-(3-bromo-2-oxopropyl)isoindoline-1,3-dione (12 g, 39 mmol), 3-bromo-5-cyclopropylpyridin-2-amine (7.5 g, 35 mmol) and DIPEA (9.2 mL, 53 mmol) in 1,4-dioxane (350 mL) was heated at 100° C. for 16 h. The mixture was cooled to room temperature and the volume was reduced by a half by concentrating in vacuo. The mixture was diluted with DCM (200 mL) and washed with NaHCO₃ solution (sat. aq., 150 mL) and brine (150 mL). The organics were dried over MgSO₄ and concentrated in vacuo. The residue was purified by column chromatography on silica gel, eluting with a gradient of 0-100% EtOAc in cyclohexane to give the title compound (7.2 g, 52%). ESI-MS (M+H)⁺: 396.1, 398.1, ¹H NMR (400 MHz, DMSO) δ8.35 (d, J=1.0 Hz, 1H), 7.99-7.94 (m, 2H), 7.94-7.91 (m, 3H), 7.39 (d, J=1.5 Hz, 1H), 4.94 (s, 2H), 2.02-1.94 (m, 1H), 0.96 (m, 2H), 0.76-0.71 (m, 2H).

Synthesis of (8-bromo-6-cyclopropylimidazo[1,2-a]pyridin-2-yl)methanamine. A mixture of 2-((8-bromo-6-cyclopropylimidazo[1,2-a]pyridin-2-yl)methyl)isoindoline-1,3-dione (1.0 g, 2.5 mmol) and hydrazine monohydrate (0.79 mL, 13 mmol) in EtOH (25 mL) was stirred at 80° C. for 1 h. The mixture was cooled and filtered through Celite®. The residue was dissolved in a mixture of 3:1 DCM/MeOH, loaded onto an SCX cartridge, which was washed with the same solvent mixture and eluted with a mixture of 3:1DCM/7 N NH₃ in MeOH. The eluent was concentrated in vacuo to give the title compound (700 mg, quant.). ESI-MS (M+H)⁺: 266.2, 268.2.

Synthesis of tert-butyl ((8-bromo-6-cyclopropylimidazo[1,2-a]pyridin-2-yl)methyl)carbamate. A mixture of (8-bromo-6-cyclopropylimidazo[1,2-a]pyridin-2-yl)methanamine (700 mg, 2.6 mmol), di-tert-butyl dicarbonate (0.73 mL, 3.2 mmol) and Et₃N (0.55 mL, 4.0 mmol) in DCM (20 mL) was stirred at room temperature for 2 h. The reaction mixture was diluted with DCM (50 mL), washed with NaHCO₃ (sat. aq., 50 mL) and brine (sat. aq., 100 mL). The organics were dried over MgSO₄ and concentrated in vacuo. The residue was purified by column chromatography on silica gel, eluting with a gradient of 0-100% EtOAc in isohexane to give the title compound (760 mg, 79%). ESI-MS (M+H)⁺: 366.3, 368.3, ¹H NMR (400 MHz, CDCl₃) δ7.83 (s, 1H), 7.52 (s, 1H), 7.20 (d, J=1.5 Hz, 1H), 5.23 (s, 1H), 4.46 (d, J=5.9 Hz, 2H), 1.91-1.83 (m, 1H), 1.53-1.52 (m, 9H), 1.00-0.94 (m, 2H), 0.70-0.65 (m, 2H).

Synthesis of tert-butyl ((6-cyclopropyl-8-(4-methylpiperazin-1-yl)imidazo[1,2-a]pyridin-2-yl)methyl)carbamate. A mixture of 1-methylpiperazine (0.35 mL, 3.1 mmol), tert-butyl ((8-bromo-6-cyclopropylimidazo[1,2-a]pyridin-2-yl)methyl)carbamate (760 mg, 2.1 mmol), Xantphos (240 mg, 0.42 mmol) and Cs₂CO₃ (1400 mg, 4.2 mmol) in 1,4-dioxane (20 mL) was degassed for 5 min with N₂. Pd(OAc)₂ (190 mg, 0.21 mmol) was added and the reaction mixture was stirred at 100° C. for 3 h. The mixture was cooled to room temperature, filtered through Celite® and concentrated in vacuo. The residue was purified by silica gel column chromatography eluting with a gradient of 0-20% 7 N NH₃ in MeOH in DCM to give the title compound (560 mg, 70%). ESI-MS (M+H)⁺: 386.5, ¹H NMR (400 MHz, CDCl₃) δ7.49-7.48 (m, 1H), 7.32 (s, 1H), 6.20-6.19 (m, 1H), 5.19 (s, 1H), 4.43-4.39 (m, 2H), 3.52-3.49 (m, 4H), 2.72-2.66 (m, 4H), 2.39 (s, 3H), 1.87-1.79 (m, 1H), 1.45 (s, 9H), 0.94-0.87 (m, 2H), 0.67-0.61 (m, 2H).

Synthesis of (6-cyclopropyl-8-(4-methylpiperazin-1-yl)imidazo[1,2-a]pyridin-2-yl)methanamine. A mixture of tert-butyl ((6-cyclopropyl-8-(4-methylpiperazin-1-yl)imidazo[1,2-a]pyridin-2-yl)methyl)carbamate (560 mg, 1.5 mmol) and TFA (2.2 mL, 29 mmol) in DCM (15 mL) was stirred at room temperature for 16 h. The reaction mixture was concentrated in vacuo and loaded onto an SCX cartridge, which was washed with 25% MeOH in DCM and eluted with 25% 7 N NH₃ in MeOH in DCM. The eluent was concentrated in vacuo to give the title compound (400 mg, 96%). ESI-MS (M+H)⁺: 286.4, ¹H NMR (400 MHz, CDCl₃) δ7.51-7.49 (m, 1H), 7.29-7.28 (m, 1H), 6.19 (d, J=1.4 Hz, 1H), 3.96 (s, 2H), 3.53 (s, 4H), 2.72-2.66 (m, 4H), 2.39-2.38 (m, 3H), 1.88-1.78 (m, 1H), 0.94-0.87 (m, 2H), 0.67-0.62 (m, 2H). 2 Exchangeable protons not observed.

### Synthesis of 6-bromo-N-((6-cyclopropyl-8-(4-methylpiperazin-1-yl)imidazo[1,2-a]pyridin-2-yl)methyl)pyrimidin-4-amine

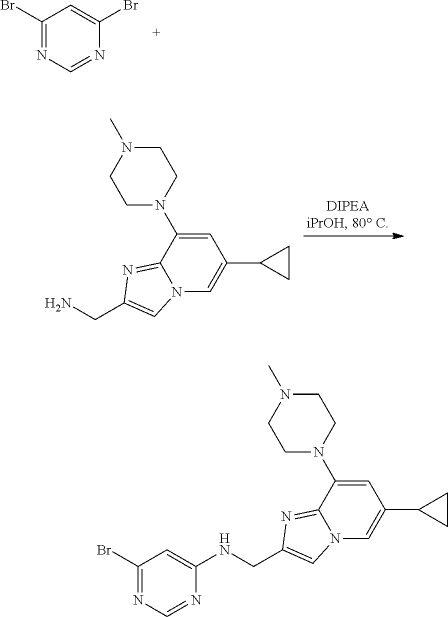 [SMILES: Brc1cc(Br)ncn1 ; CN1CCN(c2cc(C3CC3)cn3cc(CN)nc23)CC1 ; CN1CCN(c2cc(C3CC3)cn3cc(CNc4cc(Br)ncn4)nc23)CC1]

A mixture of 4,6-dibromopyrimidine (0.18 g, 0.74 mmol), (6-cyclopropyl-8-(4-methylpiperazin-1-yl)imidazo[1,2-a]pyridin-2-yl)methanamine (0.20 g, 0.70 mmol) and DIPEA (0.24 mL, 1.4 mmol) in iPrOH (5.0 mL) was stirred at 80° C. for 4 h. The mixture was cooled and concentrated in vacuo. The residue was purified by column chromatography on silica gel, eluting with 0-20% NH₃ in MeOH in DCM to give the title compound (0.095 g, 31%) which was used directly in the next step. ESI-MS (M+H)⁺: 442.3.

### Synthesis of ethyl 4-(2-(7-chloroimidazo[1,5-a]pyridin-1-yl)acetamido)-6-(((6-cyclopropylimidazo[1,2-a]pyridin-2-yl)methyl)amino)pyrimidine-2-carboxylate

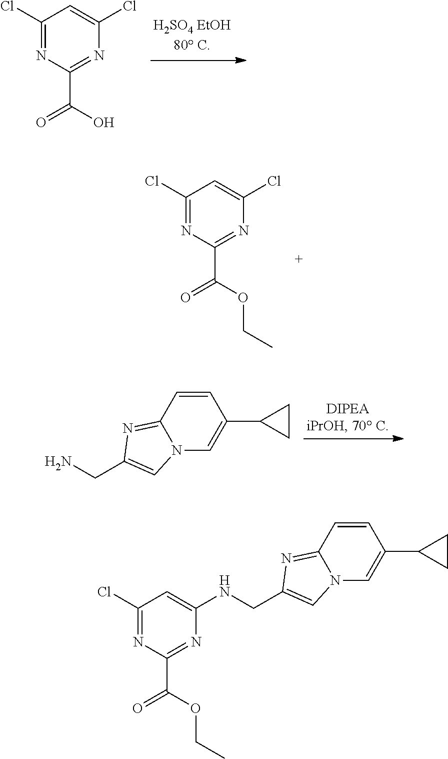 [SMILES: O=C(O)c1nc(Cl)cc(Cl)n1 ; CCOC(=O)c1nc(Cl)cc(Cl)n1 ; NCc1cn2cc(C3CC3)ccc2n1 ; CCOC(=O)c1nc(Cl)cc(NCc2cn3cc(C4CC4)ccc3n2)n1]

Synthesis of ethyl 4,6-dichloropyrimidine-2-carboxylate. A mixture of 4,6-dichloropyrimidine-2-carboxylic acid (0.30 g, 1.6 mmol) and H₂SO₄ (0.008 mL, 0.16 mmol) in EtOH (8.0 mL) was stirred at 80° C. for 18 h. The mixture was concentrated in vacuo. The residue was redissolved in DCM, washed with NaHCO₃ (sat. aq.) and concentrated in vacuo to give the title compound (0.27 g, 78%) as a colourless oil, which was used directly in the next step.

Synthesis of ethyl 4-chloro-6-(((6-cyclopropylimidazo[1,2-a]pyridin-2-yl)methyl)amino)pyrimidine-2-carboxylate. A mixture of ethyl 4,6-dichloropyrimidine-2-carboxylate (0.17 g, 0.77 mmol), (6-cyclopropylimidazo[1,2-a]pyridin-2-yl)methanamine (0.12 g, 0.64 mmol) and DIPEA (0.28 mL, 1.6 mmol) in iPrOH (10 mL) was stirred at 70° C. for 1 h. The mixture was diluted with DCM (100 mL) and washed with water (30 mL), passed through a phase separator cartridge and concentrated in vacuo. The residue was purified by column chromatography on silica gel, eluting with 0-100% DCM in EtOAc to give the title compound (0.20 g, 85%) as an off-white solid. ESI-MS (M+H)⁺: 372, ¹H NMR (400 MHz, CD₃OD) δ 8.18 (s, 1H), 7.73 (s, 1H), 7.43-7.39 (m, 1H), 7.13-7.07 (m, 1H), 6.74-6.69 (m, 1H), 4.81 (s, 2H), 4.43 (q, J=7.2 Hz, 2H), 2.00-1.92 (m, 1H), 1.45-1.40 (m, 3H), 1.02-0.97 (m, 2H), 0.76-0.71 (m, 2H). 1 exchangeable proton not observed.

### Synthesis of N⁵-((6-cyclopropylimidazo[1,2-a]pyridin-2-yl)methyl)pyridazine-3,5-diamine

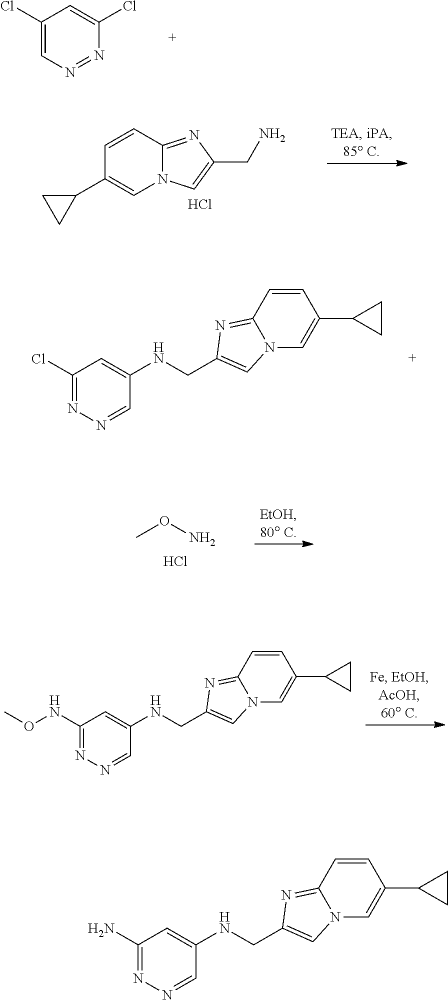

Synthesis of 6-chloro-N-((6-cyclopropylimidazo[1,2-a]pyridin-2-yl)methyl)pyridazin-4-amine. 3,5-Dichloropyridazine (240 mg, 1.61 mmol) was added to a suspension of (6-cyclopropylimidazo[1,2-a]pyridin-2-yl)methanamine hydrochloride (300 mg, 1.34 mmol) and TEA (0.56 mL, 4.02 mmol) in iPA (3 mL) and the resultant mixture was heated to 85° C. for 18 h. The mixture was diluted with water, filtered and the solid was dried to give the title compound (265 mg, 66%) as a brown solid. ESI-MS (M+H)⁺: 300.0/302.0, ¹H NMR (400 MHz, DMSO) δ8.67-8.61 (m, 1H), 8.36-8.32 (m, 1H), 7.90 (s, 1H), 7.76 (s, 1H), 7.45-7.38 (m, 1H), 7.04-6.97 (m, 1H), 6.85-6.81 (m, 1H), 4.46 (d, J=4.9 Hz, 2H), 1.98-1.90 (m, 1H), 0.97-0.88 (m, 2H), 0.69-0.64 (m, 2H).

Synthesis of N-((6-cyclopropylimidazo[1,2-a]pyridin-2-yl)methyl)-6-(methoxyamino)pyridazin-4-amine. O-Methylhydroxylamine hydrochloride (1.09 g, 13.0 mmol) was added to a solution of 6-chloro-N-((6-cyclopropylimidazo[1,2-a]pyridin-2-yl)methyl)pyridazin-4-amine (260 mg, 0.867 mmol) in EtOH (14 mL) and heated to 80° C. for 18 h. The mixture was concentrated in vacuo, dissolved into DCM and washed with NaHCO₃ (aq. sat.). The organic phase was concentrated in vacuo to give the title compound (263 mg, 53%) as a brown solid. ESI-MS (M+H)⁺: 311.2

Synthesis of N⁵-((6-cyclopropylimidazo[1,2-a]pyridin-2-yl)methyl)pyridazine-3,5-diamine. Iron powder (215 mg, 3.85 mmol) was added to a mixture of N-((6-cyclopropylimidazo[1,2-a]pyridin-2-yl)methyl)-6-(methoxyamino)pyridazin-4-amine (240 mg, 0.771 mmol) and AcOH (aq. 20%, 1.8 mL) in EtOH (12 mL) and the mixture heated to 60° C. for 18 h. An additional amount of iron powder (215 mg, 3.85 mmol) was added and heating continued for a further 4 h. The mixture was filtered through Celite® and concentrated in vacuo. Purification by column chromatography on silica gel, eluting with a gradient of 0-10% 7N NH₃/MeOH in DCM gave the title compound (91 mg, 45%). ESI-MS (M+H)⁺: 281.2, ¹H NMR (400 MHz, DMSO) δ8.34-8.31 (m, 1H), 8.02 (d, J=2.3 Hz, 1H), 7.67-7.65 (m, 1H), 7.42-7.38 (m, 1H), 7.07 (t, J=5.5 Hz, 1H), 6.99 (q, J=3.6 Hz, 1H), 5.78 (s, 2H), 5.73 (d, J=2.1 Hz, 1H), 4.35-4.31 (m, 2H), 1.93-1.90 (m, 1H), 0.96-0.89 (m, 2H), 0.71-0.64 (m, 2H).

### Synthesis of N⁴-((6-cyclopropylimidazo[1,2-a]pyridin-2-yl)methyl)pyrimidine-4,6-diamine

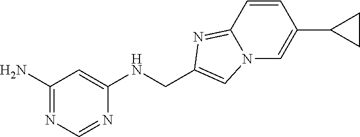 [SMILES: Nc1cc(NCc2cn3cc(C4CC4)ccc3n2)ncn1]

The title compound (32 mg, 63%) was prepared using a similar procedure to that used for N⁵-((6-cyclopropylimidazo[1,2-a]pyridin-2-yl)methyl)pyridazine-3,5-diamine using 4,6-dichloropyrimidine and (6-cyclopropylimidazo[1,2-a]pyridin-2-yl)methanamine hydrochloride as the coupling partners. ¹H NMR (400 MHz, DMSO) δ8.35-8.32 (m, 1H), 7.94-7.91 (m, 1H), 7.64-7.60 (m, 1H), 7.43-7.37 (m, 1H), 7.11-7.07 (m, 1H), 7.00 (d, J=9.1 Hz, 1H), 6.16-6.10 (m, 2H), 5.49-5.45 (m, 1H), 4.51-4.44 (m, 2H), 1.98-1.90 (m, 1H), 0.98-0.91 (m, 2H), 0.70-0.69 (m, 2H).

### Synthesis of 5-((6-cyclopropylimidazo[1,2-a]pyridin-2-yl)methoxy)pyridazin-3-amine

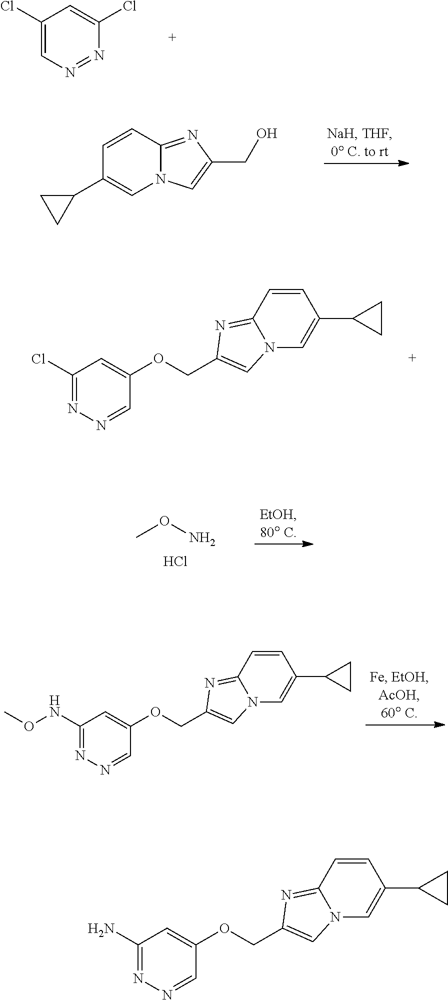

Synthesis of 2-(((6-chloropyridazin-4-yl)oxy)methyl)-6-cyclopropylimidazo[1,2-a]pyridine. Sodium hydride (60% in mineral oil, 145 mg, 3.61 mmol) was added to a cooled solution of (6-cyclopropylimidazo[1,2-a]pyridin-2-yl)methanol (400 mg, 2.13 mmol) at 0° C. in DMF (8 ml) and stirred for 2 h. A solution of 3,5-dichloropyridazine (380 mg, 2.55 mmol) in DMF (1 mL) was added and the mixture was allowed to warm to room temperature and stirred for 18 h. The mixture was diluted with water (40 mL), extracted with DCM (200 mL), the organic phase was passed through a phase separator cartridge and concentrated in vacuo. Purification by column chromatography on silica gel, eluting with a gradient of 0-100% EtOAc in DCM gave the title compound (290 mg, 45%). ¹H NMR (400 MHz, CDCl₃) δ8.91 (d, J=2.5 Hz, 1H), 7.89 (s, 1H), 7.57 (s, 1H), 7.49 (d, J=9.3 Hz, 1H), 7.18 (d, J=2.5 Hz, 1H), 7.00 (dd, J=1.5, 9.3 Hz, 1H), 5.34 (s, 2H), 1.94-1.86 (m, 1H), 1.02-0.95 (m, 2H), 0.72-0.65 (m, 2H).

Synthesis of N-(5-((6-cyclopropylimidazo[1,2-a]pyridin-2-yl)methoxy)pyridazin-3-yl)-O— methylhydroxylamine. O-Methylhydroxylamine hydrochloride (833 mg, 9.98 mmol) was added to a solution of 2-(((6-chloropyridazin-4-yl)oxy)methyl)-6-cyclopropylimidazo[1,2-a]pyridine (200 mg, 0.665 mmol) in EtOH and heated to 85° C. for 18 h. The mixture was diluted with DCM (100 mL) and washed with NaHCO₃ (aq. sat. 50 mL), passed through a phase separator cartridge and concentrated in vacuo. Purification by column chromatography on silica gel, eluting with a gradient of 0-10% MeOH in DCM gave the title compound (110 mg, 53%). ¹H NMR (400 MHz, CDCl₃) δ9.31 (s, 1H), 7.89-7.87 (m, 1H), 7.54 (s, 1H), 7.47 (d, J=9.3 Hz, 1H), 7.15 (s, 1H), 6.99-6.94 (m, 1H), 5.94-5.90 (m, 1H), 5.08 (s, 2H), 3.80-3.79 (m, 3H), 1.94-1.85 (m, 1H), 1.01-0.94 (m, 2H), 0.71-0.65 (m, 2H).

Synthesis of 5-((6-cyclopropylimidazo[1,2-a]pyridin-2-yl)methoxy)pyridazin-3-amine. Iron powder (215 mg, 3.85 mmol) was added to a mixture of N-(5-((6-cyclopropylimidazo[1,2-a]pyridin-2-yl)methoxy)pyridazin-3-yl)-O-methylhydroxylamine (240 mg, 0.771 mmol) and AcOH (aq. 20%, 1.8 mL) in EtOH (12 mL) and the mixture was heated to 60° C. for 18 h. An additional amount of iron powder (215 mg, 3.85 mmol) was added and heating continued for a further 4 h. The mixture was filtered through Celite® and concentrated in vacuo. Purification by column chromatography on silica gel, eluting with a gradient of 0-10% 7N NH₃/MeOH in DCM gave the title compound (91 mg, 45%). ¹H NMR (400 MHz, CD₃OD) δ 8.24 (s, 1H), 8.22 (d, J=2.6 Hz, 1H), 7.88-7.87 (m, 1H), 7.47-7.44 (m, 1H), 7.17-7.13 (m, 1H), 6.55-6.54 (m, 1H), 5.29-5.28 (m, 2H), 2.02-1.94 (m, 1H), 1.04-0.98 (m, 2H), 0.78-0.73 (m, 2H). 2 exchangeable protons not observed.

### Synthesis of ethyl 3-(2-(aminomethyl)-6-cyclopropylimidazo[1,2-a]pyridin-8-yl)propanoate

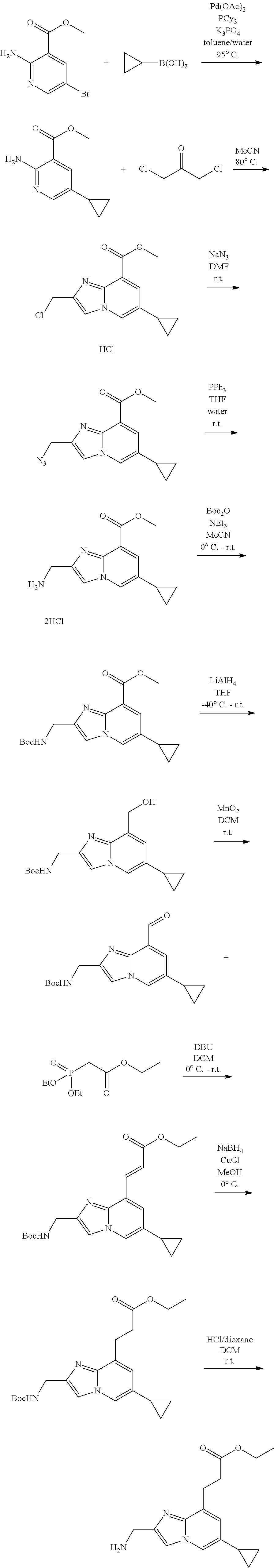

Synthesis of methyl 2-amino-5-cyclopropylnicotinate. A mixture of methyl 2-amino-5-bromonicotinate (10 g, 43 mmol), cyclopropylboronic acid (9.3 g, 110 mmol), PCy₃ (1.2 g, 4.3 mmol), and K₃PO₄ (32 g, 150 mmol) in toluene (220 mL) and water (22 mL) was degassed with nitrogen then Pd(OAc)₂ (490 mg, 2.2 mmol) was added. The mixture was stirred at 95° C. for 18 h under a nitrogen atmosphere. The reaction was cooled then diluted with water (100 mL) and EtOAc (100 mL). The mixture was separated, brine (sat. aq., 100 mL) was added to the aqueous layer which was further extracted with EtOAc (2×100 mL). The combined organic layers were filtered through Celite® and concentrated in vacuo. The residue was purified by column chromatography on silica gel, eluting with a gradient of 0-10% MeOH in DCM to give the title compound (3.4 g, 41%) as a pale green solid. ESI-MS (M+H)⁺: 193.2, ¹H NMR (400 MHz, DMSO) δ8.06 (d, J=2.5 Hz, 1H), 7.70 (d, J=2.5 Hz, 1H), 6.95 (br s, 2H), 3.80 (s, 3H), 1.87-1.80 (m, 1H), 0.88-0.83 (m, 2H), 0.59-0.55 (m, 2H).

Synthesis of methyl 2-(chloromethyl)-6-cyclopropylimidazo[1,2-a]pyridine-8-carboxylate hydrochloride. A mixture of methyl 2-amino-5-cyclopropylnicotinate (2.5 g, 13 mmol) and 1,3-dichloroacetone (2.5 g, 20 mmol) in MeCN (44 mL) was stirred at reflux for 18 h under a nitrogen atmosphere. The mixture was cooled, and the precipitate was collected by filtration and washed with Et₂O to give the title compound (3.4 g, 88%) as a cream solid. ¹H NMR (400 MHz, DMSO) δ8.97 (d, J=1.4 Hz, 1H), 8.37 (s, 1H), 8.24 (s, 1H), 5.08 (s, 2H), 4.02 (s, 3H), 2.24-2.15 (m, 1H), 1.12-1.06 (m, 2H), 0.88-0.82 (m, 2H).

Synthesis of methyl 2-(azidomethyl)-6-cyclopropylimidazo[1,2-a]pyridine-8-carboxylate. Sodium azide (1.1 g, 17 mmol) was added to a mixture of methyl 2-(chloromethyl)-6-cyclopropylimidazo[1,2-a]pyridine-8-carboxylate hydrochloride (3.4 g, 11 mmol) and NEt₃ (2.6 g, 26 mmol) in DMF (25 mL). The mixture was stirred at room temperature under a nitrogen atmosphere for 72 h. The mixture was poured into EtOAc (330 mL) and washed with water (330 mL), brine (sat. aq., 330 mL), water (330 mL), dried over MgSO₄, filtered and concentrated in vacuo to give the title compound (2.7 g, 77%) as a brown oil. ESI-MS (M+H)⁺: 272.2, ¹H NMR (400 MHz, DMSO) δ8.62 (dd, J=0.6, 1.8 Hz, 1H), 7.96 (m, 1H), 7.64 (d, J=1.9 Hz, 1H), 4.55 (s, 2H), 3.89 (s, 3H), 2.06-1.99 (m, 1H), 0.99-0.93 (m, 2H), 0.75-0.70 (m, 2H).

Synthesis of methyl 2-(aminomethyl)-6-cyclopropylimidazo[1,2-a]pyridine-8-carboxylate dihydrochloride. A mixture of methyl 2-(azidomethyl)-6-cyclopropylimidazo[1,2-a]pyridine-8-carboxylate (2.7 g, 10 mmol) and triphenylphosphine (5.3, 20 mmol) in THF (45 mL) and water (5.0 mL) was stirred at room temperature for 18 h. The mixture was concentrated in vacuo then dissolved in DCM and treated with HCl (4.0 M in 1,4-dioxane). The precipitate was collected by filtration and washed with Et₂O to give the title compound (2.4 g, 76%) as a cream solid. ESI-MS (M+H)⁺: 246.2, ¹H NMR (400 MHz, DMSO) δ8.98 (s, 1H), 8.62 (br s, 3H), 8.25 (s, 1H), 8.12 (s, 1H), 4.36 (s, 2H), 4.00 (s, 3H), 2.18-2.14 (m, 1H), 1.10-1.04 (m, 2H), 0.86-0.82 (m, 2H).

Synthesis of methyl 2-(((tert-butoxycarbonyl)amino)methyl)-6-cyclopropylimidazo[1,2-a]pyridine-8-carboxylate. Di-tert-butyl dicarbonate (1.7 g, 7.6 mmol) was added to a mixture of methyl 2-(aminomethyl)-6-cyclopropylimidazo[1,2-a]pyridine-8-carboxylate dihydrochloride (2.4 g, 7.6 mmol) and NEt₃ (5.3 mL, 38 mmol) in MeCN (25 mL) 0° C. The mixture was allowed to warm to room temperature and stirred for 18 h under a nitrogen atmosphere. The mixture was diluted with NaHCO₃ (sat. aq., 50 mL) and water (50 mL) then extracted with DCM (100 mL×3). The combined organic layers were dried over MgSO₄, filtered and concentrated in vacuo to the title compound (2.2 g, 82%) as a yellow gum. ESI-MS (M+H)⁺: 346.2, ¹H NMR (400 MHz, DMSO) δ8.59 (s, 1H), 7.70 (s, 1H), 7.36-7.34 (m, 1H), 4.23 (d, J=5.6 Hz, 2H), 3.88 (s, 3H), 2.04-1.95 (m, 1H), 1.40 (s, 9H), 0.98-0.91 (m, 2H), 0.73-0.67 (m, 2H), exchangeable NH not observed.

Synthesis of tert-butyl ((6-cyclopropyl-8-(hydroxymethyl)imidazo[1,2-a]pyridin-2-yl)methyl)carbamate. LiAlH₄ (1.0 M in THF, 6.3 mL, 6.3 mmol) was added dropwise to a solution of methyl 2-(((tert-butoxycarbonyl)amino)methyl)-6-cyclopropylimidazo[1,2-a]pyridine-8-carboxylate (2.2 g, 6.3 mmol) in THF (41 mL) at −40° C. under a nitrogen atmosphere. The mixture was stirred at −40° C. for 30 min then warmed to 0° C. and stirred for 30 min then warmed to room temperature and stirred for 30 min. The mixture was cooled to 0° C. and further LiAlH₄ (1.0 M in THF, 3.2 mL, 3.2 mmol) was added. The mixture was warmed to room temperature and stirred for 45 min. The mixture was cooled to 0° C. and water (1.5 mL) and NaOH (20% aq., 0.35 mL) were added. The mixture was warmed to room temperature and stirred for 15 min. Et₂O (10 mL) was added and the mixture was filtered through Celite® with THF and concentrated in vacuo. The residue was redissolved in THF (21 mL) and cooled to −40° C. under a nitrogen atmosphere. LiAlH₄ (1.0 M in THF, 2.8 mL, 2.8 mmol) was added dropwise and the mixture was stirred at −40° C. for 1.5 h. Further LiAlH₄ (1.0 M in THF, 2.8 mL, 2.8 mmol) was added and the mixture was stirred at −40° C. for a further 30 min. The mixture was warmed to room temperature and stirred for 2 h. The mixture was cooled to 0° C. and water (0.21 mL), NaOH (20% aq., 0.16 mL) and water (0.66 mL) were added slowly. The mixture was warmed to room temperature and stirred for 15 min. MgSO₄ was added and the mixture was filtered through Celite® with THF and concentrated in vacuo to give the title compound (1.5 g, 75%) as a yellow gum. ESI-MS (M+H)⁺: 318.2, ¹H NMR (400 MHz, DMSO) δ8.22 (s, 1H), 7.58 (s, 1H), 7.29-7.26 (m, 1H), 6.97-6.95 (m, 1H), 5.30 (t, J=5.7 Hz, 1H), 4.75 (d, J=5.3 Hz, 2H), 4.19 (d, J=5.9 Hz, 2H), 1.97-1.89 (m, 1H), 1.40 (s, 9H), 0.95-0.89 (m, 2H), 0.68-0.63 (m, 2H).

Synthesis of tert-butyl ((6-cyclopropyl-8-formylimidazo[1,2-a]pyridin-2-yl)methyl)carbamate. To a solution of tert-butyl ((6-cyclopropyl-8-(hydroxymethyl)imidazo[1,2-a]pyridin-2-yl)methyl)carbamate (400 mg, 1.3 mmol) in DCM (15 mL) was added MnO₂ (1.1 g, 13 mmol). The mixture was stirred at room temperature for 2 h under a nitrogen atmosphere. The mixture was filtered through Celite® which was then washed with DCM then THF (×3). The combined filtrates were concentrated in vacuo to give the title compound (210 mg, 32%) as a yellow oil. ESI-MS (M+H)⁺: 316.2, ¹H NMR (400 MHz, DMSO) δ10.48 (s, 1H), 8.70 (s, 1H), 7.75 (s, 1H), 7.42-7.36 (m, 1H), 6.87 (s, 1H), 4.27 (d, J=5.9 Hz, 2H), 2.08-1.99 (m, 1H), 1.41 (s, 9H), 1.01-0.94 (m, 2H), 0.77-0.71 (m, 2H).

Synthesis of ethyl (E)-3-(2-(((tert-butoxycarbonyl)amino)methyl)-6-cyclopropylimidazo[1,2-a]pyridin-8-yl)acrylate. DBU (85 μl, 0.57 mmol) was added slowly to a solution of tert-butyl ((6-cyclopropyl-8-formylimidazo[1,2-a]pyridin-2-yl)methyl)carbamate (180 mg, 0.57 mmol) and triethyl phosphonoacetate (140 mg, 0.628 mmol) in DCM (10 mL) at 0° C. under a nitrogen atmosphere. The mixture was stirred at room temperature for 72 h. The mixture was diluted with water (20 mL) and brine (sat. aq., 30 mL) and extracted with DCM (3×50 mL). The combined organic layers were passed through a phase separator and concentrated in vacuo. The residue was purified by column chromatography on silica gel, eluting with 1-5% NH₃ in MeOH in DCM to give the title compound (270 mg, quant.) as a yellow oil which was used directly in the next step without further purification. ESI-MS (M+H)⁺: 386.3.

Synthesis of ethyl 3-(2-(((tert-butoxycarbonyl)amino)methyl)-6-cyclopropylimidazo[1,2-a]pyridin-8-yl)propanoate. NaBH₄ (240 mg, 6.2 mmol) was added to a mixture of ethyl (E)-3-(2-(((tert-butoxycarbonyl)amino)methyl)-6-cyclopropylimidazo[1,2-a]pyridin-8-yl)acrylate (240 mg, 0.62 mmol) and CuCl (62 mg, 0.62 mmol) in MeOH (10 mL) in three portions at 0° C. under a nitrogen atmosphere. The mixture was stirred at 0° C. for 3 h. The mixture was diluted with NaHCO₃ (sat. aq., 5.0 mL), water (50 mL) and NaHCO₃ (sat. aq., 45 mL) and extracted with EtOAc (3×75 mL). The combined organic layers were dried over MgSO₄, filtered and concentrated in vacuo to give the title compound (280 mg, quant.) as a yellow oil which was used directly in the next step without further purification. ESI-MS (M+H)⁺: 388.3.

Synthesis of ethyl 3-(2-(aminomethyl)-6-cyclopropylimidazo[1,2-a]pyridin-8-yl)propanoate. HCl (4.0 M in 1,4-dioxane, 0.9 mL, 3.6 mmol) was added to a solution of ethyl 3-(2-(((tert-butoxycarbonyl)amino)methyl)-6-cyclopropylimidazo[1,2-a]pyridin-8-yl)propanoate (280 mg, 0.72 mmol) in DCM (2 mL). The mixture was stirred at room temperature for 90 min then concentrated in vacuo to give the title compound (250 mg, quant.) as a yellow oil which was used without further purification. ESI-MS (M+H)⁺: 288.3.

### Synthesis of ethyl 3-(2-(aminomethyl)-6-cyclopropylimidazo[1,2-a]pyridin-8-yl)-2,2-dimethylpropanoate

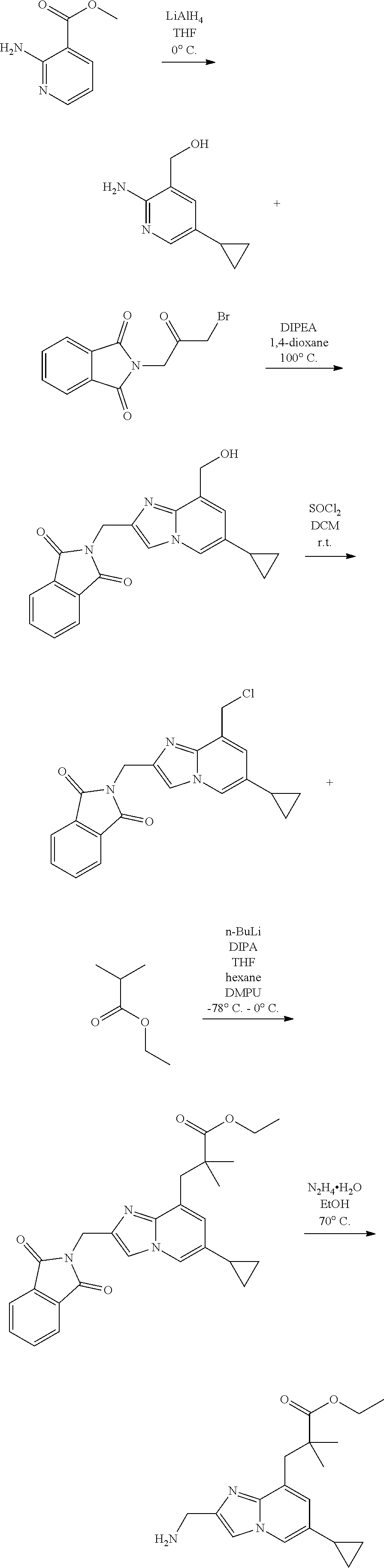

Synthesis of (2-amino-5-cyclopropylpyridin-3-yl)methanol. LiAlH₄ (2 M in THF, 20 mL, 40 mmol) was added to methyl 2-amino-5-cyclopropylnicotinate (7.6 g, 40 mmol) in THF (130 mL) cooled to 0° C. and the reaction mixture was stirred at 0° C. for 2 h. After this time water (1.5 mL) was added, followed by NaOH (20% aq., 1.2 mL), water (4.5 mL), Et₂O (200 mL) and EtOAc (150 mL). The mixture was then warmed up to room temperature. After 15 min, MgSO₄ was added and the mixture was filtered through Celite® and concentrated in vacuo. The residue was purified by column chromatography on silica gel, eluting with a gradient of 0-15% 7 N NH₃ in MeOH in DCM to give the title compound (3.7 g, 56%) as a yellow solid. ESI-MS (M+H)⁺: 165.1, ¹H NMR (400 MHz, DMSO) δ 7.71 (d, J=2.4 Hz, 1H), 7.08 (d, J=2.4 Hz, 1H), 5.43 (s, 2H), 5.12 (t, J=5.5 Hz, 1H), 4.32 (d, J=5.5 Hz, 2H), 1.82-1.74 (m, 1H), 0.86-0.81 (m, 2H), 0.56-0.51 (m, 2H).

Synthesis of 2-((6-cyclopropyl-8-(hydroxymethyl)imidazo[1,2-a]pyridin-2-yl)methyl)isoindoline-1,3-dione. A mixture of 2-(3-bromo-2-oxopropyl)isoindoline-1,3-dione (1.6 g, 5.8 mmol), (2-amino-5-cyclopropylpyridin-3-yl)methanol (0.95 g, 5.8 mmol) and DIPEA (1.0 mL, 5.8 mmol) in 1,4-dioxane (20 mL) was heated at 100° C. for 16 h. The mixture was cooled to room temperature and concentrated in vacuo. The residue was purified by column chromatography on silica gel, eluting with a gradient of 0-10% MeOH in DCM to give the title compound (2.0 g, 99%). ESI-MS (M+H)⁺: 348.2, ¹H NMR (400 MHz, DMSO) δ8.16-8.14 (m, 1H), 7.95-7.87 (m, 4H), 7.73 (s, 1H), 6.99-6.97 (m, 1H), 5.29 (t, J=5.7 Hz, 1H), 4.87 (s, 2H), 4.73 (d, J=5.6 Hz, 2H), 1.98-1.90 (m, 1H), 0.95-0.90 (m, 2H), 0.67-0.62 (m, 2H).

Synthesis of 2-((8-(chloromethyl)-6-cyclopropylimidazo[1,2-a]pyridin-2-yl)methyl)isoindoline-1,3-dione. SOCl₂ (0.15 mL, 2.0 mmol) was added to a solution of 2-((6-cyclopropyl-8-(hydroxymethyl)imidazo[1,2-a]pyridin-2-yl)methyl)isoindoline-1,3-dione (0.20 g, 0.58 mmol) in DCM (5.0 mL) and the reaction mixture was stirred at room temperature for 2 h then concentrated in vacuo. The residue was washed with NaHCO₃ (sat. aq., 25 mL) and extracted with DCM (3×25 mL). The combined organics were dried over MgSO₄ and concentrated in vacuo to give the title compound (190 mg, 92%) as a yellow solid. ¹H NMR (400 MHz, DMSO) δ8.28-8.26 (m, 1H), 7.95-7.87 (m, 4H), 7.79 (s, 1H), 7.17-7.16 (m, 1H), 4.94 (s, 2H), 4.91 (s, 2H), 1.98-1.90 (m, 1H), 0.96-0.90 (m, 2H), 0.69-0.64 (m, 2H).

Synthesis of ethyl 3-(6-cyclopropyl-2-((1,3-dioxoisoindolin-2-yl)methyl)imidazo[1,2-a]pyridin-8-yl)-2,2-dimethylpropanoate. n-BuLi (2.5 M in hexane, 1.7 mL, 4.3 mmol) was added to a solution of diisopropylamine (0.69 mL, 4.9 mmol) in THF (18 mL) under a N₂ atmosphere at −78° C. The solution was stirred at −78° C. for 30 min, then ethyl isobutyrate (0.57 mL, 4.3 mmol) was added slowly and the reaction mixture was stirred at −78° C. for 4 h. A solution of 2-((8-(chloromethyl)-6-cyclopropylimidazo[1,2-a]pyridin-2-yl)methyl)isoindoline-1,3-dione (1.2 g, 3.3 mmol) in THF (6.0 mL) and DMPU (6.0 mL) was then added slowly over 45 min and the reaction mixture was stirred at −78° C. for 90 min. The reaction mixture was then warmed up to 0° C., NH₄Cl (sat. aq., 10 mL) was added and the reaction then warmed up to room temperature. The mixture was diluted with water (100 mL) and extracted with EtOAc (3×100 mL). The combined organics were dried over MgSO₄ and concentrated in vacuo. The residue was purified by column chromatography on silica gel, eluting with a gradient of 0-80% EtOAc in cyclohexane to give the title compound (0.18 g, 12%) as a yellow oil. ESI-MS (M+H)⁺: 446.3, ¹H NMR (400 MHz, DMSO) δ8.19-8.16 (m, 1H), 7.99-7.89 (m, 4H), 7.72 (s, 1H), 6.67 (s, 1H), 4.91 (s, 2H), 4.07-4.00 (m, 2H), 3.10 (s, 2H), 1.98-1.87 (m, 1H), 1.16-1.11 (m, 9H), 1.00-0.91 (m, 2H), 0.67-0.60 (m, 2H).

Synthesis of ethyl 3-(2-(aminomethyl)-6-cyclopropylimidazo[1,2-a]pyridin-8-yl)-2,2-dimethylpropanoate. A mixture of ethyl 3-(6-cyclopropyl-2-((1,3-dioxoisoindolin-2-yl)methyl)imidazo[1,2-a]pyridin-8-yl)-2,2-dimethylpropanoate (180 mg, 0.39 mmol) and hydrazine monohydrate (49 μL, 0.79 mmol) in EtOH (4.0 mL) was stirred at 70° C. for 24 h. The mixture was cooled and loaded onto an SCX cartridge, washed with 50% MeOH in DCM and eluted with 50% 7 N NH₃ in MeOH in DCM. The eluent was concentrated in vacuo to give the title compound (110 mg, 89%) as a yellow oil. ESI-MS (M+H)⁺: 316.3, ¹H NMR (400 MHz, DMSO) δ8.24 (s, 1H), 7.66 (s, 1H), 6.64 (s, 1H), 4.08 (q, J=7.1 Hz, 2H), 3.83 (s, 2H), 3.22 (s, 2H), 3.17 (s, 2H), 1.97-1.88 (m, 1H), 1.22-1.17 (m, 9H), 1.00-0.92 (m, 2H), 0.68-0.63 (m, 2H).

### Synthesis of tert-butyl 3-(2-(aminomethyl)-6-cyclopropylimidazo[1,2-a]pyridin-8-yl)-2,2-dimethylpropanoate

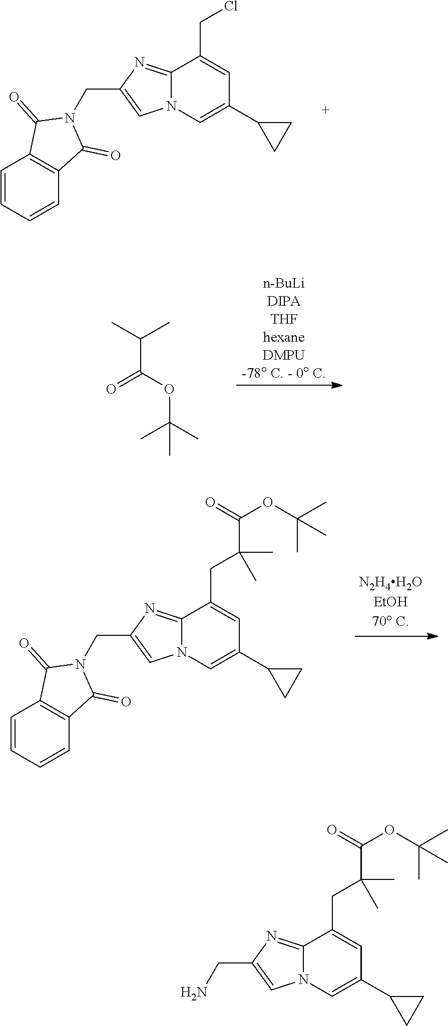

Synthesis of tert-butyl 3-(6-cyclopropyl-2-((1,3-dioxoisoindolin-2-yl)methyl)imidazo[1,2-a]pyridin-8-yl)-2,2-dimethylpropanoate. A solution of diisopropylamine (0.41 mL, 4.1 mmol) in anhydrous THF (10 mL) was placed under a N₂ atmosphere and cooled to −78° C., n-BuLi (2.5 M in hexane, 1.3 mL, 3.2 mmol) was then added slowly and the reaction mixture was stirred at −78° C. for 30 min. Tert-butyl 2-methylpropanoate (0.59 mL, 3.6 mmol) was then added and the resulting mixture stirred at −78° C. for 1 h. A solution of 2-((8-(chloromethyl)-6-cyclopropylimidazo[1,2-a]pyridin-2-yl)methyl)isoindoline-1,3-dione (1.0 g, 2.7 mmol) in anhydrous THF (5.0 mL) and DMPU (5.0 mL) was then added dropwise and the reaction mixture was stirred at −78° C. for 2 h. The reaction mixture was then warmed up to room temperature and NH₄C₁ (sat. aq., 10 mL) was added. The mixture was diluted with water (100 mL) and extracted with EtOAc (3×100 mL). The combined organics were dried over MgSO₄ and concentrated in vacuo. The residue was purified by column chromatography on silica gel, eluting with a gradient of 20-80% EtOAc in cyclohexane to give the title compound was further purified by crystallising from EtOH to afford the title compound (0.085 g, 7%) as a white solid. ESI-MS (M+H)⁺: 474.4.

Synthesis of tert-butyl 3-(2-(aminomethyl)-6-cyclopropylimidazo[1,2-a]pyridin-8-yl)-2,2-dimethylpropanoate. A mixture of tert-butyl 3-(6-cyclopropyl-2-((1,3-dioxoisoindolin-2-yl)methyl)imidazo[1,2-a]pyridin-8-yl)-2,2-dimethylpropanoate (0.08 g, 0.17 mmol) and hydrazine hydrate (0.021 mL, 0.34 mmol) in ethanol (2 mL) was stirred at 70° C. for 24 h. Further hydrazine hydrate (0.042 mL, 0.68 mmol) was added and the mixture stirred at 70° C. for a further 3 h. The reaction was cooled to room temperature and then loaded onto an SCX cartridge. The cartridge was washed with 50% MeOH in DCM and eluted with 50% 7 N NH₃ in MeOH in DCM. The eluent was concentrated in vacuo to give the title compound (110 mg, 89%) as a yellow oil. ESI-MS (M+H)⁺: 344.3.

Synthesis of (2-(aminomethyl)-6-cyclopropylimidazo[1,2-a]pyridin-8-yl)methanol. A mixture of 2-((6-cyclopropyl-8-(hydroxymethyl)imidazo[1,2-a]pyridin-2-yl)methyl)isoindoline-1,3-dione (0.50 g, 1.4 mmol) and hydrazine monohydrate (0.18 mL, 2.9 mmol) in EtOH (14 mL) was stirred at 70° C. for 18 h. The mixture was cooled to room temperature, loaded onto an SCX cartridge and washed with MeOH. The product was eluted with 7 N NH₃ in MeOH to give the title compound (0.25 g, 80%) as an orange gum. ESI-MS (M+H)⁺: 218.2, ¹H NMR (400 MHz, DMSO) δ8.21 (s, 1H), 7.65 (s, 1H), 6.95 (d, J=1.4 Hz, 1H), 4.77 (s, 2H), 3.79 (s, 2H), 3.18 (s, 1H), 1.99-1.90 (m, 1H), 0.93 (ddd, J=4.3, 6.3, 8.3 Hz, 2H), 0.69-0.64 (m, 2H). NH₂ not observed.

### Synthesis of (6-cyclopropyl-5-fluoroimidazo[1,2-a]pyridin-2-yl)methanamine

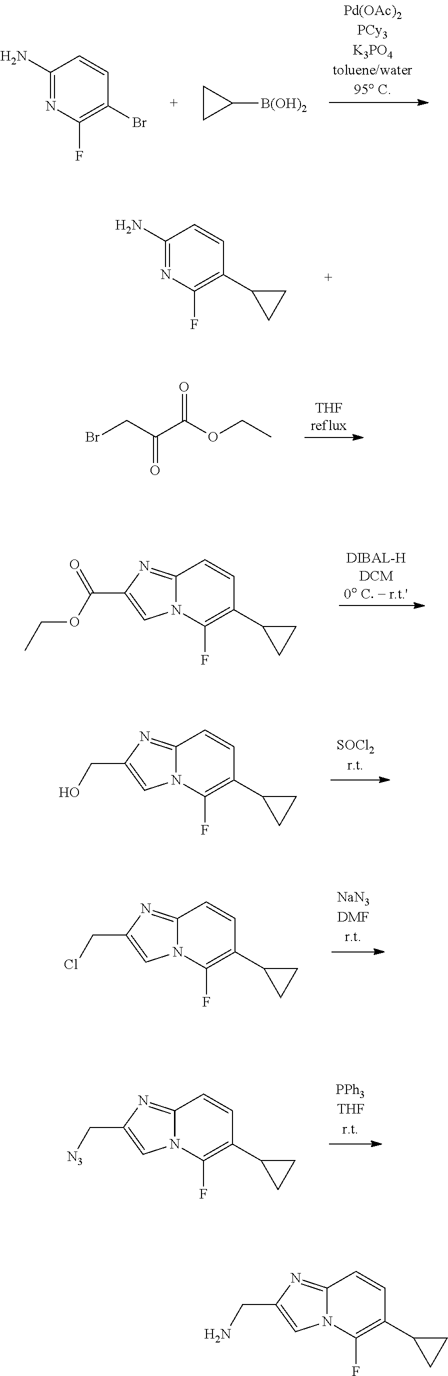

Synthesis of 5-cyclopropyl-6-fluoropyridin-2-amine. A mixture of 5-bromo-6-fluoropyridin-2-amine (2.2 g, 12 mmol), cyclopropylboronic acid (2.5 g, 29 mmol), PCy₃ (350 mg, 2.3 mmol), Pd(OAc)₂ (260 mg, 1.2 mmol) and K₃PO₄ (8.6 g, 40 mmol) in toluene (60 mL) and water (3 mL) was degassed for 10 minutes and stirred at 95° C. for 18 h. The reaction was cooled and concentrated in vacuo to half the original volume. The mixture was diluted with water (50 mL) and EtOAc (150 mL) and stirred at room temperature for 15 min. The mixture was then filtered through Celite® and separated. The organics were dried over MgSO₄ and concentrated in vacuo. The residue was purified by column chromatography on silica gel, eluting with a gradient of 10-30% EtOAc in isohexane to give the title compound (1.5 g, 86%) as a pale yellow oil. ESI-MS (M+H)⁺: 153.0, ¹H NMR (400 MHz, CDCl₃) δ7.16-7.09 (m, 1H), 6.27-6.23 (m, 1H), 4.36 (s, 2H), 1.91-1.81 (m, 1H), 0.91-0.84 (m, 2H), 0.62-0.55 (m, 2H).

Synthesis of ethyl 6-cyclopropyl-5-fluoroimidazo[1,2-a]pyridine-2-carboxylate. A mixture of 5-cyclopropyl-6-fluoropyridin-2-amine (1.5 g, 9.9 mmol) and ethyl 3-bromo-2-oxopropanoate (1.3 mL, 10 mmol) in THF (45 mL) was stirred at reflux for 24 h. The mixture was cooled and concentrated in vacuo. The residue was partitioned between DCM (200 mL) and K₂CO₃ (sat. aq., 100 mL). The organic layer was dried over MgSO₄ and concentrated in vacuo. The residue was purified by column chromatography on silica gel, eluting with a gradient of 1-5% MeOH in DCM. The residue was further purified by column chromatography on silica gel, eluting with a gradient of 25-100% EtOAc in isohexane to give the title compound (0.55 g, 22%) as a pale brown solid. ESI-MS (M+H)⁺: 249.1, ¹H NMR (400 MHz, CDCl₃) δ8.20 (s, 1H), 7.44 (d, J=9.3 Hz, 1H), 6.92 (t, J=8.6 Hz, 1H), 4.46 (q, J=7.2 Hz, 2H), 2.11-2.01 (m, 1H), 1.47-1.42 (m, 3H), 1.10-1.02 (m, 2H), 0.81-0.74 (m, 2H).

Synthesis of (6-cyclopropyl-5-fluoroimidazo[1,2-a]pyridin-2-yl)metanol. DIBAL-H (1.0 M in THF, 4.4 mL, 4.4 mmol) was added dropwise to a solution ethyl 6-cyclopropyl-5-fluoroimidazo[1,2-a]pyridine-2-carboxylate (550 mg, 2.2 mmol) in THF (15 mL) at 0° C. under a nitrogen atmosphere. Additional DIBAL-H (1.0 M in THF, 0.5 mL, 0.5 mmol) was added dropwise and the reaction mixture was stirred at room temperature for 1 h. The mixture was treated dropwise with a Rochelle's salt solution (10% aq., 3 mL), stirred at room temperature for 30 minutes and extracted with EtOAc (20 mL). The organics were dried over MgSO₄ and concentrated in vacuo. The residue was purified by column chromatography on silica gel, eluting with a gradient of 1-10% MeOH in DCM to give the title compound (330 mg, 71%) as a green gum. ESI-MS (M+H)⁺: 207.1, ¹H NMR (400 MHz, CDCl₃) δ7.57-7.56 (m, 1H), 7.31 (d, J=9.2 Hz, 1H), 6.89-6.84 (m, 1H), 4.85-4.84 (m, 2H), 2.07-2.00 (m, 1H), 1.05-0.99 (m, 2H), 0.76-0.71 (m, 2H). Exchangeable proton not observed.

Synthesis of 2-(chloromethyl)-6-cyclopropyl-5-fluoroimidazo[1,2-a]pyridine. A solution of (6-cyclopropyl-5-fluoroimidazo[1,2-a]pyridin-2-yl)methanol (150 mg, 0.73 mmol) in SOCl₂ (0.32 mL, 4.4 mmol) was stirred at room temperature for 6 h. The reaction mixture was then azeotroped with toluene (×2). The residue was washed with K₂CO₃ (dil. aq., 2 mL) and extracted with DCM (10 mL). The organic layer was dried over MgSO₄ and concentrated in vacuo to give the title compound (170 mg, quant.) as a dark brown oil which was used without further purification.

Synthesis of 2-(azidomethyl)-6-cyclopropyl-5-fluoroimidazo[1,2-a]pyridine. A mixture of 2-(chloromethyl)-6-cyclopropyl-5-fluoroimidazo[1,2-a]pyridine (160 mg, 0.73 mmol) and NaN₃ (62 mg, 0.95 mmol) in DMF (1.0 mL) was stirred at room temperature for 18 h. The mixture was diluted with water (10 mL) and extracted with EtOAc (15 mL). The organics were washed with water (2×10 mL) and brine (20 mL), dried over MgSO₄ and concentrated in vacuo to give the title compound (130 mg, 76%) as a viscous brown oil. ESI-MS (M+H)⁺: 232.1, ¹H NMR (400 MHz, CDCl₃) δ7.60 (s, 1H), 7.36-7.31 (m, 1H), 6.89 (t, J=8.6 Hz, 1H), 4.53 (s, 2H), 2.07-2.01 (m, 1H), 1.06-0.99 (m, 2H), 0.79-0.71 (m, 2H).

Synthesis of (6-cyclopropyl-5-fluoroimidazo[1,2-a]pyridin-2-yl)methanamine. A mixture of 2-(azidomethyl)-6-cyclopropyl-5-fluoroimidazo[1,2-a]pyridine (130 mg, 0.55 mmol) and triphenyl phosphine (290 mg, 1.1 mmol) in THF (5.0 mL) and water (0.5 mL) was stirred at room temperature for 6 h. The reaction mixture was concentrated in vacuo, the residue was then dissolved in DCM (10 mL), dried over MgSO₄ and concentrated in vacuo. The residue was purified by column chromatography on silica gel, eluting with a gradient of 5% MeOH in DCM to 2% 7 N NH₃ in MeOH in DCM to give the title compound (87 mg, 77%) as a green-brown solid. ESI-MS (M+H)⁺: 206.1, ¹H NMR (400 MHz, CDCl₃) δ7.50-7.49 (m, 1H), 7.28 (d, J=9.3 Hz, 1H), 6.87-6.82 (m, 1H), 4.03-4.03 (m, 2H), 2.06-1.99 (m, 1H), 1.04-0.98 (m, 2H), 0.76-0.71 (m, 2H). 2 exchangeable protons not observed.

### Synthesis of N⁴-((8-(((tert-butyldiphenylsilyl)oxy)methyl)-6-cyclopropylimidazo[1,2-a]pyridin-2-yl)methyl)pyridine-2,4-diamine

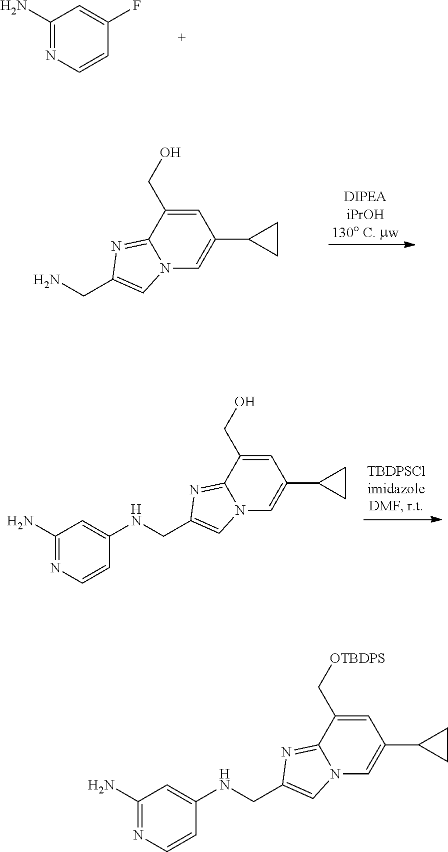

Synthesis of (2-(((2-aminopyridin-4-yl)amino)methyl)-6-cyclopropylimidazo[1,2-a]pyridin-8-yl)methanol. A mixture of 4-fluoropyridin-2-amine (0.10 g, 0.92 mmol), (2-(aminomethyl)-6-cyclopropylimidazo[1,2-a]pyridin-8-yl)methanol (0.20 g, 0.92 mmol) and DIPEA (0.24 mL, 1.4 mmol) in iPrOH (2.0 mL) was stirred at 130° C. in a microwave for 2 h. The mixture was concentrated in vacuo and the residue was purified by column chromatography on silica gel, eluting with 10-50% NH₃ in MeOH in DCM to give the title compound (0.14 g, 51%) as an orange gum. ESI-MS (M+H)⁺: 310.2, ¹H NMR (400 MHz, DMSO) δ8.22-8.20 (m, 1H), 7.61 (s, 1H), 7.47-7.45 (m, 1H), 6.99 (d, J=1.5 Hz, 1H), 6.59 (t, J=5.8 Hz, 1H), 5.92 (dd, J=2.1, 5.9 Hz, 1H), 5.61-5.60 (m, 1H), 5.35-5.29 (m, 3H), 4.80-4.77 (m, 2H), 4.31-4.28 (m, 2H), 1.97-1.89 (m, 1H), 0.95-0.89 (m, 2H), 0.68-0.63 (m, 2H).

Synthesis of N⁴-((8-(((tert-butyldiphenylsilyl)oxy)methyl)-6-cyclopropylimidazo[1,2-a]pyridin-2-yl)methyl)pyridine-2,4-diamine. TBDPSCl (0.14 mL, 0.54 mmol) was added to a mixture of (2-(((2-aminopyridin-4-yl)amino)methyl)-6-cyclopropylimidazo[1,2-a]pyridin-8-yl)methanol (0.15 g, 0.47 mmol) and imidazole (0.064 g, 0.94 mmol) in DMF (3.0 mL). The mixture was stirred at room temperature for 18 h. Further TBDPSCl (0.14 mL, 0.54 mmol) and imidazole (0.064 g, 0.94 mmol) were added and the mixture was stirred at room temperature for 24 h. Water (15 mL) and brine (sat. aq., 25 mL) were added and the mixture was extracted with EtOAc (3×50 mL). The combined organic layers were dried (MgSO₄), filtered and concentrated in vacuo. The residue was purified by column chromatography on silica gel, eluting with 1-20% NH₃ in MeOH in DCM to give the title compound (0.15 g, 57%) as a yellow solid. ¹H NMR (400 MHz, DMSO) δ8.27 (s, 1H), 7.69 (dd, J=1.7, 7.8 Hz, 4H), 7.62-7.61 (m, 1H), 7.51-7.42 (m, 7H), 7.14-7.09 (m, 1H), 6.51 (t, J=5.7 Hz, 1H), 5.87 (dd, J=2.0, 5.8 Hz, 1H), 5.56 (d, J=2.0 Hz, 1H), 5.25 (s, 2H), 5.03 (s, 2H), 4.24-4.21 (m, 2H), 2.01-1.93 (m, 1H), 1.10 (s, 9H), 0.99-0.93 (m, 2H), 0.66-0.61 (m, 2H).

### Synthesis of 6-((6-cyclopropylimidazo[1,2-a]pyridin-2-yl)methyl)pyrimidin-4-amine

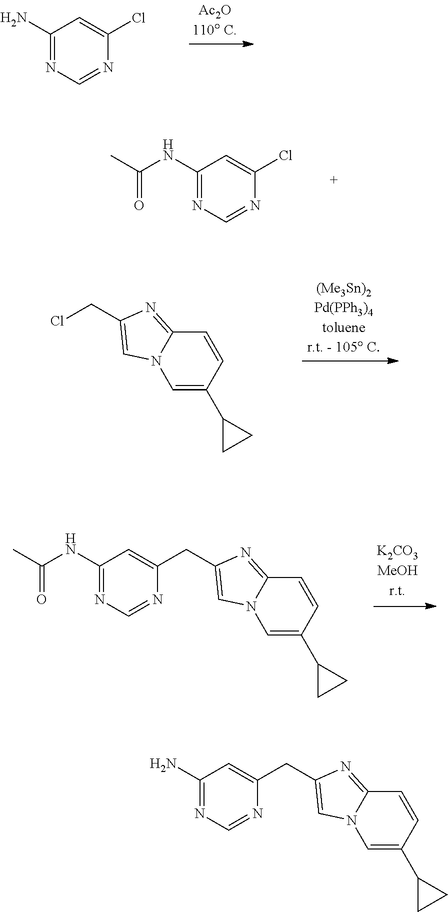 [SMILES: Nc1cc(Cl)ncn1 ; CC(=O)Nc1cc(Cl)ncn1 ; ClCc1cn2cc(C3CC3)ccc2n1 ; CC(=O)Nc1cc(Cc2cn3cc(C4CC4)ccc3n2)ncn1 ; Nc1cc(Cc2cn3cc(C4CC4)ccc3n2)ncn1]

Synthesis of N-(6-chloropyrimidin-4-yl)acetamide. 6-Chloropyrimidin-4-amine (5.0 g, 39 mmol) was stirred in Ac₂O (35 mL) at 110° C. for 6 h. The mixture was cooled and diluted with EtOAc (150 mL). The mixture was washed with NaHCO₃ (sat. aq., 100 mL) and brine (sat. aq., 100 mL), dried (MgSO₄), filtered and concentrated in vacuo. The residue was purified by column chromatography on silica gel, eluting with 30-80% EtOAc in cyclohexane to give the title compound (4.3 g, 80%) as a white powder. ¹H NMR (400 MHz, DMSO) δ11.21 (s, 1H), 8.74 (s, 1H), 8.09 (s, 1H), 2.15 (s, 3H).

Synthesis of N-(6-((6-cyclopropylimidazo[1,2-a]pyridin-2-yl)methyl)pyrimidin-4-yl)acetamide. A mixture of N-(6-chloropyrimidin-4-yl)acetamide (0.40 g, 2.3 mmol) and hexamethylditin (0.48 mL, 2.3 mmol) in toluene (12 mL) was degassed with N₂ and Pd(PPh₃)₄ (0.27 g, 0.23 mmol) was added. The mixture was stirred at 100° C. for 18 h under a N₂ atmosphere. The mixture was cooled and added to a stirred mixture of 2-(chloromethyl)-6-cyclopropylimidazo[1,2-a]pyridine (0.48 g, 2.3 mmol) in toluene (11 mL). The mixture was degassed with N₂ and stirred at 105° C. for 18 h under a N₂ atmosphere. The mixture was concentrated in vacuo and the residue was purified by column chromatography on silica gel, eluting with 1-10% NH₃ in MeOH in DCM to give the title compound (0.043 g, 6%) as a yellow gum. ESI-MS (M+H)⁺: 308.2, ¹H NMR (400 MHz, DMSO) δ10.82 (s, 1H), 8.76 (d, J=1.3 Hz, 1H), 8.32-8.31 (m, 1H), 7.97 (d, J=1.1 Hz, 1H), 7.68-7.66 (m, 1H), 7.38-7.35 (m, 1H), 6.99-6.95 (m, 1H), 4.11 (s, 2H), 2.10-2.10 (m, 3H), 1.97-1.86 (m, 1H), 0.96-0.90 (m, 2H), 0.71-0.66 (m, 2H).

Synthesis of 6-((6-cyclopropylimidazo[1,2-a]pyridin-2-yl)methyl)pyrimidin-4-amine. A mixture of N-(6-((6-cyclopropylimidazo[1,2-a]pyridin-2-yl)methyl)pyrimidin-4-yl)acetamide (0.28 g, 0.91 mmol) and K₂CO₃ (0.255 g, 1.8 mmol) in MeOH (15 mL) was stirred at room temperature for 18 h. The mixture was concentrated in vacuo. Water (50 mL) was added and the mixture was extracted with MeOH in DCM (1:9, 4×50 mL). The combined organic layers were dried (MgSO₄), filtered and concentrated in vacuo to give the title compound (0.17 g, 69%) as an orange gum, which was used without further purification. ESI-MS (M+H)⁺: 266.2.

### Synthesis of (6-chloropyrimidin-4-yl)(6-cyclopropylimidazo[1,2-a]pyridin-2-yl)methanone

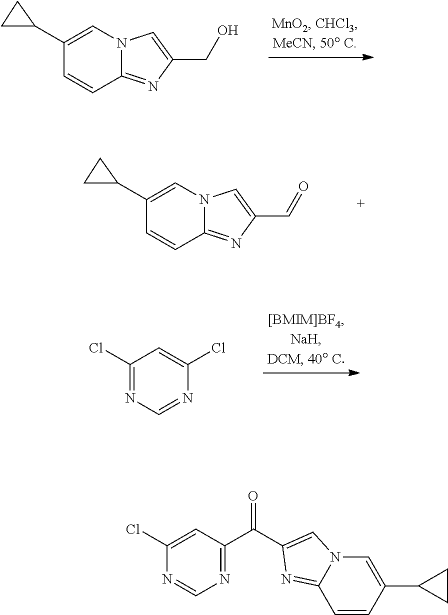 [SMILES: OCc1cn2cc(C3CC3)ccc2n1 ; O=Cc1cn2cc(C3CC3)ccc2n1 ; Clc1cc(Cl)ncn1 ; O=C(c1cc(Cl)ncn1)c1cn2cc(C3CC3)ccc2n1]

Synthesis of 6-cyclopropylimidazo[1,2-a]pyridine-2-carbaldehyde. A suspension of (6-cyclopropylimidazo[1,2-a]pyridin-2-yl)methanol (2.0 g, 11 mmol) and manganese(IV) oxide (9.2 g, 110 mmol) in chloroform (25 mL) and MeCN (25 mL) was stirred at 50° C. for 1 h. The mixture was cooled to room temperature, filtered through Celite® and concentrated in vacuo to give the title compound (1.3 g, 66%) which was used without further purification. ESI-MS (M+H)⁺: 187.2, ¹H NMR (400 MHz, CDCl₃) δ10.13 (s, 1H), 8.06 (s, 1H), 7.93 (s, 1H), 7.57 (d, J=9.6 Hz, 1H), 7.03 (dd, J=1.8, 9.3 Hz, 1H), 1.95-1.87 (m, 1H), 1.05-0.99 (m, 2H), 0.75-0.69 (m, 2H).

Synthesis of (6-chloropyrimidin-4-yl)(6-cyclopropylimidazo[1,2-a]pyridin-2-yl)methanone. 6-Cyclopropylimidazo[1,2-a]pyridine-2-carbaldehyde (3.1 g, 16.7 mmol), 4,6-dichloropyrimidine (1.9 g, 13 mmol), and 1-butyl-3-methylimidazolium tetrafluoroborate (1.0 mL, 5.5 mmol) were dissolved in DCM (200 mL) under N₂. Sodium hydride (60% in mineral oil, 0.67 g, 17 mmol) was added portionwise and the resulting reaction mixture was heated to 40° C. for 3 h. The reaction mixture was allowed to cool to room temperature and poured into ice cold water (100 mL) and saturated brine solution (100 mL). The solution was extracted with DCM (100 mL) and the organic layer was dried over MgSO₄. The solvent was removed under reduced pressure and the residue purified by silica gel column chromatography (eluting with a gradient of cyclohexane->100% EtOAc). The crude title compound was further purified by silica gel column chromatography, eluting with an isocratic gradient of 10% MeOH/NH₃ in DCM to give the title compound (900 mg, 23%) which was used without further purification. ¹H NMR (400 MHz, CDCl₃): δ ppm 9.20 (1H, d, J=1.0 Hz), 8.87 (1H, d, J=0.9 Hz), 8.24 (1H, d, J=1.0 Hz), 7.95 (1H, ddd, J=0.9, 0.9, 1.6 Hz), 7.61 (1H, ddd, J=0.9, 1.0, 9.5 Hz), 7.04 (1H, dd, J=1.6, 9.5 Hz), 1.96-1.88 (1H, m), 1.06-1.00 (2H, m), 0.76-0.71 (2H, m).

### Synthesis of 1-(6-cyclopropylimidazo[1,2-a]pyridin-2-yl)ethanol

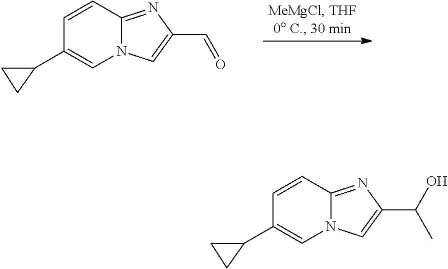

To a solution of 6-cyclopropylimidazo[1,2-a]pyridine-2-carbaldehyde (6) (1.2 g, 6.45 mmol) in anhydrous tetrahydrafuran (30 mL) was added MeMgBr (3M, 3.2 mL, 9.68 mmol) dropwise at 0° C. The reaction mixture was stirred at 0° C. for 3 hrs, then the reaction mixture was quenched with saturated NH₄Cl solution (10 mL) and extracted with ethyl acetate (30 mL×3). The combined organic layers were washed with brine, dried over Na₂SO₄, and concentrated to give the crude product, which was purified by silica gel column chromatography using 10% of MeOH in DCM to give the title compound (836 mg, yield: 64%) as a pale yellow solid. ESI-MS (M+H)⁺: 203.0. ¹HNMR (400 Hz, DMSO) δ8.31 (t, J=1.2 Hz, 1H), 7.62 (s, 1H), 7.35 (d, J=9.2 Hz, 1H), 6.93 (dd, J=10.0, 2.0 Hz, 1H), 5.14 (d, J=4.8 Hz, 1H), 4.84-4.75 (m, 1H), 1.96-1.86 (m, 1H), 1.40 (d, J=6.4 Hz, 3H), 0.96-0.88 (m, 2H), 0.70-0.64 (m, 2H).

### Synthesis of 1-(6-cyclopropylimidazo[1,2-a]pyridin-2-yl)ethan-1-amine

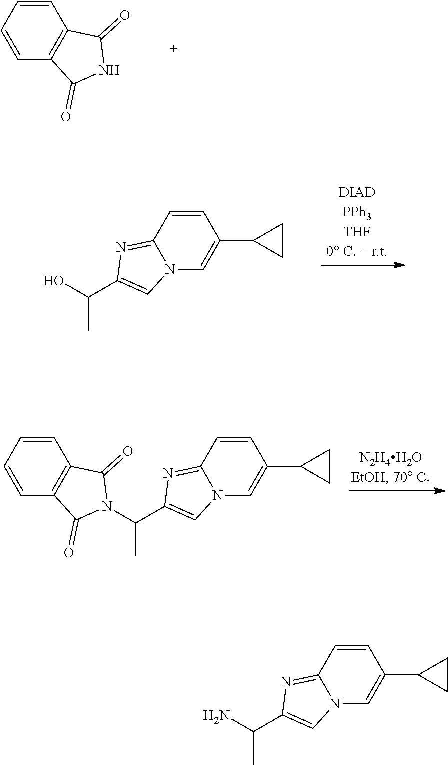 [SMILES: O=C1NC(=O)c2ccccc21 ; CC(O)c1cn2cc(C3CC3)ccc2n1 ; CC(c1cn2cc(C3CC3)ccc2n1)N1C(=O)c2ccccc2C1=O ; CC(N)c1cn2cc(C3CC3)ccc2n1]

Synthesis of 2-(1-(6-cyclopropylimidazo[1,2-a]pyridin-2-yl)ethyl)isoindoline-1,3-dione. A mixture of 1-(6-cyclopropylimidazo[1,2-a]pyridin-2-yl)ethan-1-ol (140 mg, 0.71 mmol), phthalimide (120 mg, 0.78 mmol) and PPh₃ (280 mg, 1.1 mmol) in THF (20 mL) was cooled to 0° C. and DIAD (0.21 mL, 1.1 mmol) was added. The reaction mixture was then stirred at 0° C. for 30 min, then at room temperature for 20 h. The mixture was diluted with EtOAc (20 mL) and washed with water (20 mL) and brine (sat. aq., 50 mL). The organics were dried over MgSO₄ and concentrated in vacuo. The residue was purified by column chromatography on silica gel, eluting with a gradient of 1-10% MeOH in DCM to give the title compound (290 mg, quant.) as a yellow gum. ESI-MS (M+H)⁺: 332.1

Synthesis of 1-(6-cyclopropylimidazo[1,2-a]pyridin-2-yl)ethan-1-amine. A mixture of 2-(1-(6-cyclopropylimidazo[1,2-a]pyridin-2-yl)ethyl)isoindoline-1,3-dione (240 mg, 0.71 mmol) and hydrazine monohydrate (44 μL, 1.4 mmol) in MeOH (5.0 mL) was stirred at reflux for 6 h. The mixture was cooled to room temperature and concentrated in vacuo. The residue was purified by column chromatography on silica gel, eluting with a gradient of 1-10% MeOH in DCM then 1-10% 7 N NH₃ in MeOH in DCM to give the title compound (25 g, 17%) as a brown gum. ESI-MS (M+H)⁺: 202.2, ¹H NMR (400 MHz, CDCl₃) δ7.85 (s, 1H), 7.44 (d, J=9.3 Hz, 1H), 7.37 (s, 1H), 6.93-6.88 (m, 1H), 4.27 (q, J=6.6 Hz, 1H), 1.92-1.83 (m, 1H), 1.52 (d, J=6.5 Hz, 3H), 0.99-0.91 (m, 2H), 0.70-0.62 (m, 2H). 2 exchangeable protons not observed

### Synthesis of 2-((6-chloropyrimidin-4-yl)difluoromethyl)-6-cyclopropylimiazo[1,2-a]pyridine

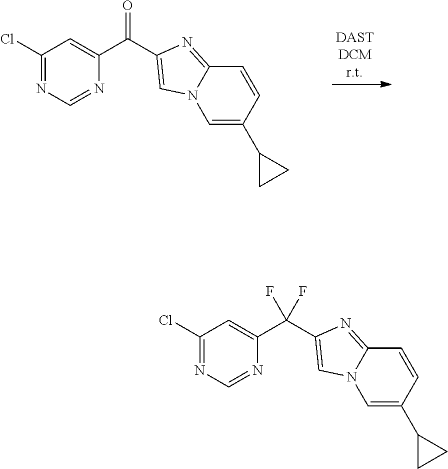 [SMILES: O=C(c1cc(Cl)ncn1)c1cn2cc(C3CC3)ccc2n1 ; FC(F)(c1cc(Cl)ncn1)c1cn2cc(C3CC3)ccc2n1]

A solution of (6-chloropyrimidin-4-yl)(6-cyclopropylimidazo[1,2-a]pyridin-2-yl)methanone (0.18 g, 0.60 mmol) in DCM (2.0 mL) was degassed with N₂. DAST (0.20 mL, 1.5 mmol) was added dropwise to the stirred solution under a N₂ atmosphere at room temperature. The mixture was stirred at room temperature for 4 h. Further DAST (0.20 mL, 1.5 mmol) was added and the mixture was stirred at room temperature for 18 h. The mixture was added dropwise to ice water (20 mL) and extracted with DCM (3×30 mL). The combined organic layers were concentrated in vacuo. The residue was purified by column chromatography on silica gel, eluting with 5-95% EtOAc in cyclohexane to give the title compound (0.10 g, 52%). ¹H NMR (400 MHz, CDCl₃) δ9.03 (s, 1H), 7.94-7.89 (m, 3H), 7.49-7.46 (m, 1H), 7.02-6.98 (m, 1H), 1.95-1.86 (m, 1H), 1.02-0.97 (m, 2H), 0.71-0.66 (m, 2H).

### Synthesis of (6-chloropyrimidin-4-yl)(6-cyclopropylimidazo[1,2-a]pyridin-2-yl)methanol

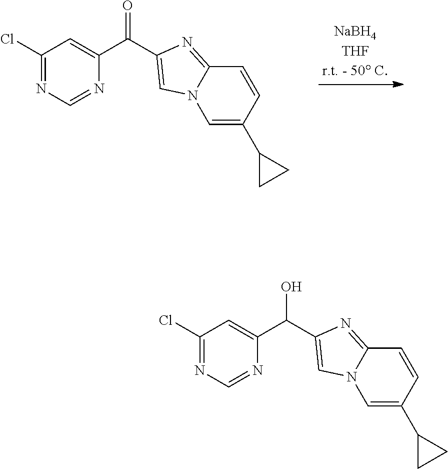 [SMILES: O=C(c1cc(Cl)ncn1)c1cn2cc(C3CC3)ccc2n1 ; OC(c1cc(Cl)ncn1)c1cn2cc(C3CC3)ccc2n1]

NaBH₄ (32 mL) was added portion wise over 10 min to a solution of (6-chloropyrimidin-4-yl)(6-cyclopropylimidazo[1,2-a]pyridin-2-yl)methanone (0.10 g, 0.34 mmol) in THF (10 mL) under a N₂ atmosphere. The mixture was stirred at 50° C. for 30 min. The mixture was cooled and water (5.0 mL) was added. The mixture was extracted with DCM (3×10 mL) and the combined organic layers were passed through a phase separator and concentrated in vacuo. The residue was purified by column chromatography on silica gel, eluting with 0-30% EtOAc in cyclohexane to give the title compound (0.033 g, 32%). ESI-MS (M+H)⁺: 301.0.

### Synthesis of tert-butyl (5-(aminomethyl)-4,6-dimethylpyridin-2-yl)(tert-butoxycarbonyl)carbamate

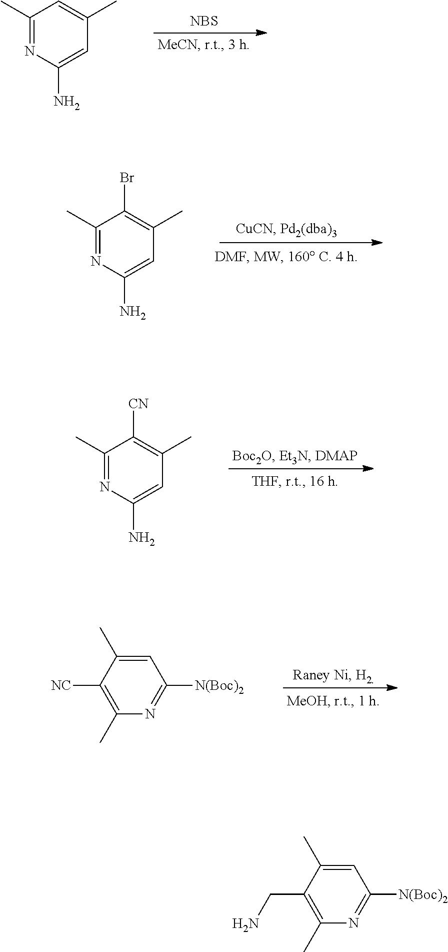

Synthesis of 5-bromo-4,6-dimethylpyridin-2-amine. To a solution of 4,6-dimethylpyridin-2-amine (5.0 g, 41 mmol) in CH₃CN (100 mL) was added a solution of NBS (7.3 g, 41 mmol) in CH₃CN (50 mL) at room temperature. The reaction mixture was stirred at room temperature for 3 h. Water (200 mL) was added and extracted with EtOAc (100 mL×3). The combined organic layers were washed with brine (100 mL), dried over anhydrous Na₂SO₄, and concentrated to give the crude product, which was purified by column chromatography (eluent: PE/EtOAc=5/1) to give 5-bromo-4,6-dimethylpyridin-2-amine (7.0 g, yield: 85%) as a yellow solid. ESI-MS [M+H]⁺: 201.2.

Synthesis of 6-amino-2,4-dimethylnicotinonitrile. A mixture of 5-bromo-4,6-dimethylpyridin-2-amine (2.0 g, 10 mmol), CuCN (1.8 g, 20 mmol) and Pd₂(dba)₃ (458 mg, 0.5 mmol) in DMF (20 mL) was stirred in a sealed tube. After degassing with N₂ for 1 min, the reaction was irradiated in microwave at 160° C. for 4 h. After the reaction mixture was cooled to room temperature, the reaction mixture was filtered through Celite® and the filter cake was washed with DCM/MeOH (10/1, 50 mL). The filtrate was concentrated to give the crude, which was purified by column chromatography (eluent: PE/EtOAc=5/1) give 6-amino-2,4-dimethylnicotinonitrile (1.1 g, yield: 75%) as a yellow solid. ESI-MS [M+H]+: 148.2.

Synthesis of tert-butyl (tert-butoxycarbonyl)(5-cyano-4,6-dimethylpyridin-2-yl)carbamate. A mixture of 6-amino-2,4-dimethylnicotinonitrile (1.1 g, 7.5 mmol), Boc₂O (4.9 g, 22.5 mmol), DMAP (92 mg, 0.75 mmol) and Et₃N (3.0 g, 30 mmol) in THF (30 mL) was stirred at room temperature for 16 h. The mixture was concentrated to give the crude product, which was purified by column chromatography (eluent: PE/EtOAc=10/1) to give tert-butyl (tert-butoxycarbonyl)(5-cyano-4,6-dimethylpyridin-2-yl)carbamate (2.2 g, yield: 85%) as a white solid. ESI-MS [M+H]⁺: 348.2.

Synthesis of tert-butyl (5-(aminomethyl)-4,6-dimethylpyridin-2-yl)(tert-butoxycarbonyl)carbamate. To a solution of tert-butyl (tert-butoxycarbonyl)(5-cyano-4,6-dimethylpyridin-2-yl)carbamate (2.2 g mg, 6.3 mmol) in MeOH (20 mL) was added Raney-Ni. After the reaction mixture was stirred at room temperature for 1 h under H₂, the mixture was filtered through Celite® and the filter cake was washed with DCM/MeOH (10/1, 100 mL). The filtrate was concentrated to give to give the crude, which was purified by column chromatography (eluent: DCM/MeOH=30/1) to give t tert-butyl (5-(aminomethyl)-4,6-dimethylpyridin-2-yl)(tert-butoxycarbonyl)carbamate (1.7 g, yield: 77%) as a yellow solid. ESI-MS [M+H]⁺: 352.2.

### Synthesis of 4-(aminomethyl)-3,5-dimethylbenzonitrile

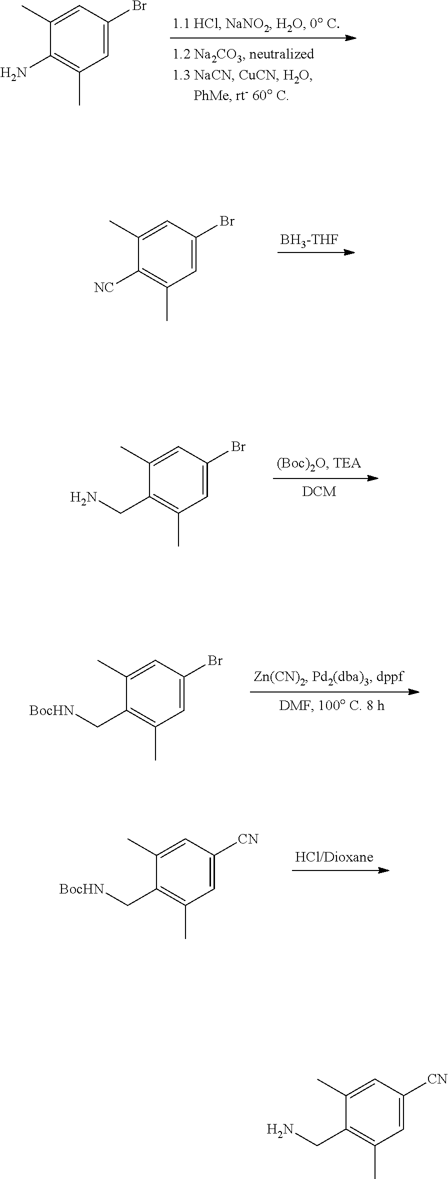

Synthesis of 4-bromo-2,6-dimethylbenzonitrile. To a suspended solution of 4-Bromo-2,6-dimethyl-phenylamine (4.92 g, 24.6 mmol) in HCl (2M aq., 50 ml) at 0° C. was added a solution of sodium nitrite (1.7 g, 25 mmol) in water (10 mL) and the reaction mixture was stirred at 0° C. for 45 minutes. After the reaction was neutralized with solid Na₂CO₃ at 0° C., the resulting solution was added in portions to a solution of sodium cyanide (3.8 g, 78 mmol) and copper cyanide (2.7 g, 30 mmol) in toluene/water (50 mL/10 mL). The resulting mixture was stirred at 0° C. for 1 h and then slowly warmed up to 60° C. and stirred for another 2.5 hours. Then reaction was cooled to room temperature and concentrated, the aqueous phase was extracted with EtOAc (100 mL×3). The combined organic layers were washed with brine, dried over Na₂SO₄, concentrated in vacuo to give the crude, which was purified by column chromatography on silica gel (eluent: EtOAc/PE: 1/1) to give the 4-bromo-2,6-dimethylbenzonitrile (4.1 g, yield: 79%) as a yellow oil. ESI-MS [M+H]⁺: 210.2.

Synthesis of (4-bromo-2,6-dimethylphenyl)methanamine. To a solution of 4-bromo-2,6-dimethylbenzonitrile (1.0 g, 4.76 mmol) in THF (30 mL) was slowly added BH₃-THF (1M, 10 mL, 10 mmol) at 0° C., the mixture was stirred at room temperature for 16 h. MeOH (20 mL) was added and the mixture was stirred for 1 h. The reaction was concentrated in vacuo to give the crude, which was purified by column chromatography on silica gel (eluent: DCM/MeOH=10/1) to give (4-bromo-2,6-dimethylphenyl)methanamine (500 mg, 49%) as an orange oil. ESI-MS [M+H]⁺: 214.2.

Synthesis of tert-butyl (4-bromo-2,6-dimethylbenzyl)carbamate. To a solution of (4-bromo-2,6-dimethylphenyl)methanamine (500 mg, 2.35 mmol) in DCM (20 mL) was added Boc₂O (770 mg, 3.52 mmol) and Et₃N (715 mg, 7.05 mmol). After the mixture was stirred at room temperature for 16 h, water (40 mL) was added and extracted with DCM (30 mL×3). The combined organic layers were washed with brine, dried with Na₂SO₄, concentrated in vacuo to give the crude, which was purified by column chromatography on silica gel (eluent: DCM/MeOH=20/1) to give tert-butyl (4-bromo-2,6-dimethylbenzyl)carbamate (700 mg, 95% yield) as a yellow solid. ESI-MS [M+H]⁺: 314.2.

Synthesis of tert-butyl (4-cyano-2,6-dimethylbenzyl)carbamate. To a solution of tert-butyl (4-bromo-2,6-dimethylbenzyl)carbamate (700 mg, 2.2 mmol) in DMF (20 mL) was added Zn(CN)₂ (516 mg, 4.4 mmol), Pd₂(dba)₃ (200 mg, 0.22 mmol) and dppf (244 mg, 0.44 mmol). The mixture was stirred at 100° C. for 8 h under N₂. Then cooled to room temperature, water (50 mL) was added and extracted with EtOAc (50 mL×3). The combined organic layers were washed with brine, dried with Na₂SO₄, concentrated in vacuo to give the crude, which was purified by column chromatography on silica gel (eluent: PE/EtOAc=3/1) to give tert-butyl (4-cyano-2,6-dimethylbenzyl)carbamate (350 mg, yield: 61%) as a yellow solid. ESI-MS [M+H]⁺: 261.2.

Synthesis of 4-(aminomethyl)-3,5-dimethylbenzonitrile. To a solution of tert-butyl (4-cyano-2,6-dimethylbenzyl)carbamate (350 mg, 1.35 mmol) in 1,4-dioxane (10 mL) was added HCl (2 mL, 4M in dioxane) and the mixture was stirred at room temperature for 2 h. The reaction was concentrated in vacuo, the residue was neutralized with NaHCO₃ (sat. aq., 20 ml), extracted with DCM/MeOH (10/1, 20 mL×3). The combined organic layers were washed with brine (20 mL), dried over Na₂SO₄ and concentrated to give the crude product, which was purified with Prep-TLC with DCM/MeOH (10/1) to give the product 4-(aminomethyl)-3,5-dimethylbenzonitrile as a white solid (150 mg, yield: 69%). ESI-MS [M+H]⁺: 161.2.

### Synthesis of 2-(5-methoxy-2-oxobenzo[d]oxazol-3 (2H)-yl)acetic acid

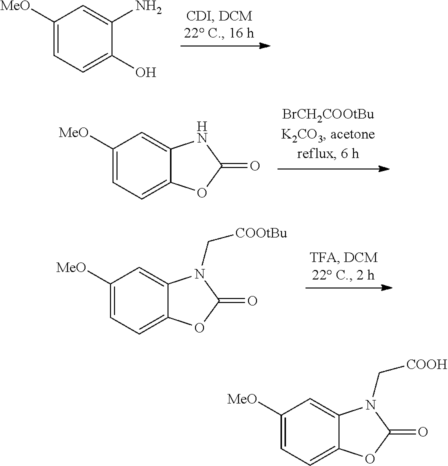

Synthesis of 5-methoxybenzo[d]oxazol-2 (3H)-one. N,N-Carbonyldiimidazole (1.61 g, 9.88 mmol) was added to a suspension of 4-methoxy-2-aminophenol (1.25 g, 8.98 mmol) in anhydrous dichloromethane (45 mL) at room temperature. The resulting solution was stirred overnight and quenched with water (20 mL) and hydrochloric acid (2M, 20 mL). The layers were separated, and the organic layer was washed again with hydrochloric acid (2M, 20 mL). The combined aqueous layers were extracted with dichloromethane. The combined organic layers were dried (MgSO₄), filtered, and silica gel was added to the filtrate and the solvent evaporated. The residue was purified by column chromatography (50 g silica gel) using a gradient of heptane:ethyl acetate=9:1 to 1:1 to afford 5-methoxybenzo[d]oxazol-2 (3H)-one (1.18 g, 80%), as a light orange solid. LCMS [System 2, 4.5 min buffered] RT=2.13 min; [M+H]⁺ 166. ¹H NMR (400 MHz, CD₃SOCD₃, ppm) δ11.55 (s, 1H), 7.18 (d, J9 Hz, 1H), 6.65 (d, J2 Hz, 1H), 6.61 (dd, J2, 9 Hz, 1H), 3.74 (s, 3H).

Synthesis of tert-butyl 2-(5-methoxy-2-oxobenzo[d]oxazol-3 (2H)-yl)acetate. tert-Butyl bromoacetate (1.27 mL, 8.57 mmol) was added to a suspension of 5-methoxybenzo[d]oxazol-2 (3H)-one (1.18 g, 7.15 mmol) and potassium carbonate (1.19 g, 17.2 mmol) in acetone (18 mL). The mixture was heated under reflux for 2.5 h. More tert-butyl bromoacetate (1.27 mL, 8.57 mmol) was added, and the mixture was heated for another 2 h. More potassium carbonate (1.19 g, 17.2 mmol) was added and the mixture was heated for another hour. The mixture was cooled to room temperature and the solvent was removed under reduced pressure. The residue was partitioned between ethyl acetate (50 mL) and water (100 mL) and the aqueous layer was extracted once with ethyl acetate (20 mL). The organic solutions were combined, washed with brine, dried (MgSO₄), filtered and evaporated to obtain an orange oil. The crude product was purified on silica gel (50 g) eluting with a gradient of heptane:ethyl acetate=8:2 to 7:3 to obtain tert-butyl 2-(5-methoxy-2-oxobenzo[d]oxazol-3 (2H)-yl)acetate (1.87 g, 94%), as pinkish solid. LC-MS [System 2, 4.5 min buffered] RT=2.80 min; [M−Bu^{t}+H]⁺224. ¹H NMR (400 MHz, CDCl₃, ppm) δ 7.11 (d, J9 Hz, 1H), 6.63 (dd, J9, 2 Hz, 1H), 6.44 (d, J2 Hz, 1H), 4.42 (s, 2H), 3.80 (s, 3H), 1.47 (s, 9H).

Synthesis of 2-(5-methoxy-2-oxobenzo[d]oxazol-3 (2H)-yl)acetic acid. Trifluoroacetic acid (10 mL) was added to a solution of tert-butyl 2-(5-methoxy-2-oxobenzo[d]oxazol-3 (2H)-yl)acetate (1.87 g, 11.3 mmol) in dichloromethane (10 mL). The resulting mixture was stirred at 22° C. for 2 h. The volatiles were removed under reduced pressure, and the residue was purified by reversed phase chromatography (Biotage SNAP ULTRA Cis, 60 g cartridge, 50 mL/min) using a gradient of A:B=8:2-1:9 over 10 column volumes, where A is 0.1% v/v formic acid in water and B is 0.1% v/v formic acid in acetonitrile). Fractions containing pure product were pooled and concentrated to obtain 2-(5-methoxy-2-oxobenzo[d]oxazol-3 (2H)-yl)acetic acid (1.10 g, 73%), as a colourless solid. UP-L^{C} [Method A1] RT=2.42 min; [M+H]⁺=224. ¹H NMR (400 MHz, CD₃SOCD₃, ppm) δ 13.32 (s, 1H), 7.26 (d, J9 Hz, 1H), 7.04 (d, J2 Hz, 1H), 6.67 (dd, J9, 2 Hz, 1H), 4.63 (s, 2H), 3.75 (s, 3H).

### Synthesis of (2R,3S)-3-(3-chlorophenyl)-2-methylbutanoic acid and (2S,3R)-3-(3-chlorophenyl)-2-methylbutanoic acid

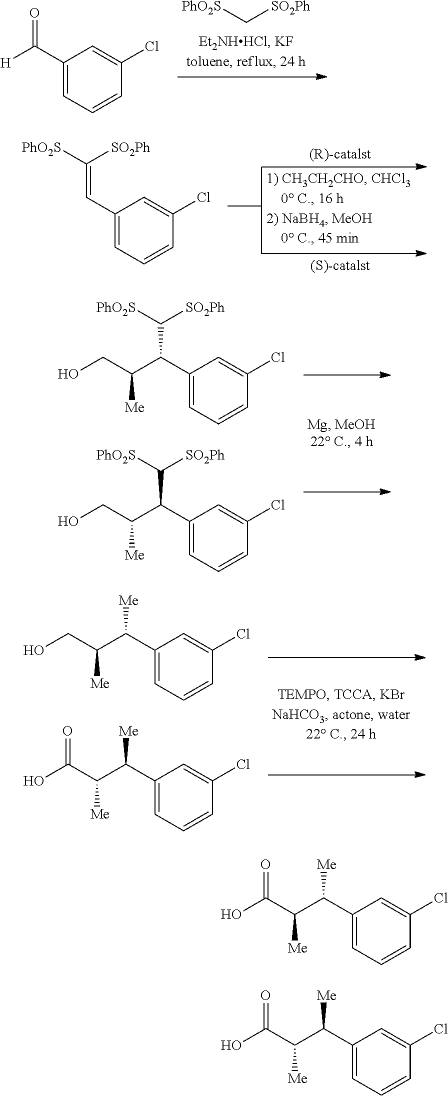

Synthesis of (2-(3-chlorophenyl)-1,1-bis(phenylsulfonyl)ethane. A mixture of 3-chloro benzaldehyde (3.86 mL 30.7 mmol), bis(phenylsulfonyl)methane (10.0 g, 33.7 mmol), diethylammonium chloride (6.39 g, 58.3 mmol) and potassium fluoride (270 mg, 4.60 mmol) in anhydrous toluene was heated under nitrogen at reflux in a flask connected to a Dean Stark water separator. After 26 h, the mixture was cooled to room temperature and the solvent was evaporated. The residue was partitioned between water (50 mL) and dichloromethane (160 mL). The aqueous layer was extracted with dichloromethane (2×50 mL). The combined organic layers were dried (Na₂SO₄), filtered, and concentrated under reduced pressure to obtain a yellow solid. A mixture of diethyl ether and dichloromethane (4:1, 40 mL) was added to the solid. The resulting suspension was filtered and the solid was washed with methyl tert-butyl ether (30 mL) to obtain (2-(3-chlorophenyl)-1,1-bis(phenylsulfonyl)ethene (5.86 g, 41%) as a colourless solid. The yellow mother liquors were concentrated under reduced pressure and purified by flash chromatography on silica gel (250 g), eluting with a gradient of heptane:ethyl acetate=85:15 to 70:30 to obtain a second batch of product (880 mg, 6%), as a yellow solid. LC-MS [System 2, 4.5 min buffered] RT=3.49 min; [M+H]⁺ 419. ¹H NMR (400 MHz, CDCl₃, ppm) δ 8.58 (s, 1H), 8.09-8.04 (m, 2H), 7.74-7.68 (m, 1H), 7.65-7.57 (m, 4H), 7.55-7.51 (m, 1H), 7.40-7.29 (m, 6H).

Synthesis of (2R,3S)-3-(3-chlorophenyl)-2-methyl-4,4-bis(phenylsulfonyl)butan-1-ol. A suspension of (2-(3-chlorophenyl)-1,1-bis(phenylsulfonyl)ethene (1.09 g, 2.29 mmol) and (R)-α,α-bis[3,5-bis(trifluoromethyl)phenyl]-2-pyrrolidinemethanol trimethylsilyl ether (137 mg, 0.23 mmol) in anhydrous chloroform (2.5 mL) was cooled to 0° C. Propionaldehyde (1.7 mL, 22.9 mmol) was added and the reaction mixture was stirred at 0° C. overnight. After 16 h, the crude mixture was combined with two other batches (from 2.29 mmol of starting material) and concentrated under reduced pressure. The residue was diluted with methanol (400 mL), and the resulting mixture was cooled in an ice-water bath. Sodium borohydride (2.60 g, 68.7 mmol) was added in portions over 15 min (the temperature rose to 10° C.) and the mixture was stirred for 45 min at 5-10° C. Hydrochloric acid (0.5 M, 320 mL) was added to quench the reaction, and ethyl acetate (500 mL) and brine (150 mL) were added. The layers were separated, and the aqueous layer was extracted with ethyl acetate (2×250 mL). The combined organic extracts were washed with water (200 mL) and brine (200 mL), dried over anhydrous Na₂SO₄ and concentrated to obtain a sticky, yellow solid (4.8 g). The crude product was dissolved in dimethylsulfoxide and purified (in three portions) by reverse-phase chromatography (Biotage Ultra Cis 120 g cartridge), eluting with water:acetonitrile=70:30 for 2 column volumes and then water:acetonitrile from 70:30 to 25:75 over 12 column volumes. The fractions from these 3 purifications which contained a mixture of isomers were combined, concentrated under reduced pressure and re-purified by reverse-phase chromatography (Biotage Ultra Cis 400 g cartridge), eluting with water:acetonitrile=65:35 for 2 column volumes and then water:acetonitrile from 65:35 to 30:70 over 12 column volumes. All the fractions with a purity >99% by L^{C} were combined and concentrated in vacuo to afford (2R,3S)-3-(3-chlorophenyl)-2-methyl-4,4-bis(phenylsulfonyl)butan-1-ol (2.20 g, 67%), as a colourless solid. LC-MS [System 2, 4.5 min buffered]: RT=3.35 min, [M−H]⁻=477. ¹H NMR (400 MHz, CDCl₃, ppm): δ7.75-7.73 (m, 2H), 7.69-7.67 (m, 2H), 7.59-7.53 (m, 2H), 7.46-7.37 (m, 5H), 7.28 (s, 1H), 7.22 (d, J4.8 Hz, 2H), 5.81 (d, J1.8 Hz, 1H), 4.17-4.12 (m, 1H), 3.80 (dd, J10.6, 1.8 Hz, 1H), 3.65-3.56 (m, 1H), 3.16-3.07 (m, 1H), 2.20 (dd, J7.0, 4.5 Hz, 1H), 0.74 (d, J6.7 Hz, 3H).

Synthesis of (2R,3S)-3-(3-chlorophenyl)-2-methylbutan-1-ol. In a dried flask filled with nitrogen, magnesium turnings (2.78 g, 24.31 mmol) and a catalytical amount of iodine (7 mg, 0.03 mmol) were stirred for 10 min. The flask was gently heated until sublimated iodine was observed. Then the flask was placed in a water bath at 20° C., and a solution of (2R,3S)-3-(3-chlorophenyl)-2-methyl-4,4-bis(phenylsulfonyl)butan-1-ol (2.23 g, 4.65 mmol) in methanol (220 mL) was added. After 3 h, the reaction mixture was filtered through a short pad of Dicalite, which was rinsed with methanol (50 mL) and methyl tert-butyl ether (100 mL). Saturated aqueous ammonium chloride (200 mL) and hydrochloric acid (2M, 50 mL) were added to the filtrate, which was extracted with methyl tert-butyl ether (2×300 mL). The combined organic solutions were washed with aqueous sodium thiosulfate (150 mL) and brine (200 mL), dried (Na₂SO₄) and concentrated under reduced pressure to afford (2R,3S)-3-(3-chlorophenyl)-2-methylbutan-1-ol (856 mg, 92%), as a yellow oil. LC-MS [System 2, 4.5 min buffered]: RT=3.05 min, no ionisation. ¹H NMR (400 MHz, CDCl₃, ppm): δ7.23-7.16 (m, 3H), 7.06 (dt, J7.3, 1.5 Hz, 1H), 3.62-3.52 (m, 2H), 2.82-2.75 (m, 1H), 1.87-1.77 (m, 1H), 1.28 (d, J6.7 Hz, 3H), 0.79 (d, J6.7 Hz, 3H).

Synthesis of (2R,3S)-3-(3-chlorophenyl)-2-methylbutanoic acid. Saturated aqueous sodium bicarbonate (13 mL) was added to a solution of (2R,3S)-3-(3-chlorophenyl)-2-methylbutan-1-ol (840 mg, 4.23 mmol) in acetone (40 mL). The resulting mixture was cooled to 0° C. Potassium bromide (101 mg, 0.85 mmol) and TEMPO (13.2 mg, 0.08 mmol) were added. Trichloroisocyanuric acid (TCCA, 1.97 g, 8.46 mmol) was added in 6 portions over 20 min. The yellow mixture stirred at 0° C. for 30 min and then stirred at 22° C. overnight. After 24 h, 2-propanol (3 mL) was added. After a few minutes a white solid precipitated. The solid was filtered off and the filtrate was concentrated under reduced pressure to remove the acetone. The residue treated with aqueous sodium carbonate (10% w/v, 15 mL) and ethyl acetate (5 mL) and filtered again to remove an insoluble white solid, which was rinsed with ethyl acetate (5 mL) and water (5 mL). The filtrate layers were separated. The aqueous layer was washed with ethyl acetate (10 mL), treated with hydrochloric acid (2M, 15 mL) to give pH 1 and extracted with ethyl acetate (2×20 mL). The organic layers were combined, dried (Na₂SO₄), filtered and concentrated in vacuo to obtain (2R,3S)-3-(3-chlorophenyl)-2-methylbutanoic acid (722 mg, 80%) as a colourless oil which crystallized on standing. UP-L^{C} [Method A1]: RT=3.13 min, [M−H]⁻=211. ¹H NIR (400 MHz, CD₃SOCD₃, ppm) δ 12.28 (s, 1H), 7.35-7.25 (m, 3H), 7.22-7.19 (m, 1H), 2.90-2.82 (m, 1H), 2.55-2.49 (m, 1H), 1.19 (d, J7.3 Hz, 3H), 0.81 (d, J7.3 Hz, 3H).

Synthesis of (2S,3R)-3-(3-chlorophenyl)-2-methylbutanoic acid. (2S,3R)-3-(3-chlorophenyl)-2-methylbutanoic acid (590 mg, 83%) was synthesized by an analogous method to (2R,3S)-3-(3-chlorophenyl)-2-methylbutanoic acid, from (2-(3-chlorophenyl)-1,1-bis(phenylsulfonyl)ethene and (S)-α,α-bis[3,5-bis(trifluoromethyl)phenyl]-2-pyrrolidinemethanol trimethylsilyl ether. UP-L^{C} [Method A1]: RT=3.16 min, [M−H]⁻=211. ¹H NIR (400 MHz, CD₃SOCD₃, ppm) δ 12.28 (s, 1H), 7.35-7.26 (m, 3H), 7.22-7.19 (m, 1H), 2.90-2.82 (m, 1H), 2.55-2.49 (m, 1H), 1.19 (d, J7.3 Hz, 3H), 0.81 (d, J7.3 Hz, 3H).

### Synthesis of 2-(3-chloro-6-fluoro-1H-indol-5-yl)acetic acid

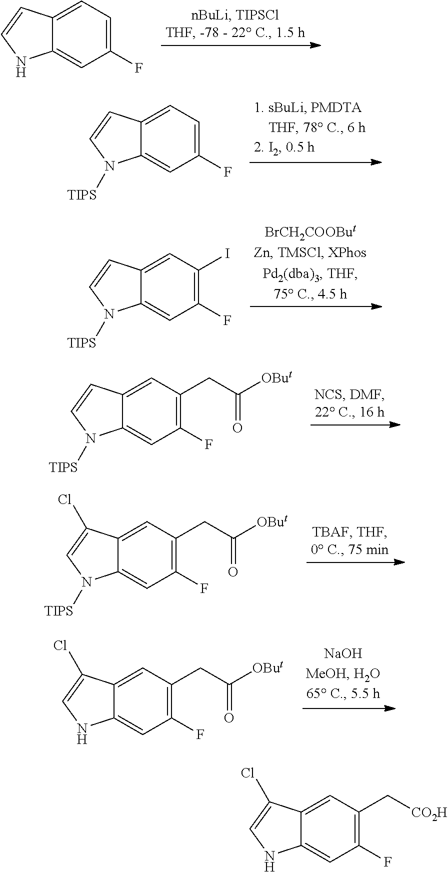

Synthesis of 6-fluoro-1-(triisopropylsilyl)-1H-indole. 6-Fluoroindole (4.54 g, 33.6 mmol) was dissolved in anhydrous tetrahydrofuran (75 mL) and cooled to −78° C. under nitrogen. n-Butyllithium (2.5 M in hexanes, 23.0 mL, 57.5 mmol) was added dropwise over 15 min and the mixture was stirred for 5 min. A solution of triisopropylsilyl chloride (8.20 mL, 38.2 mmol) in anhydrous tetrahydrofuran (15 mL) was added over 5 min. The mixture was stirred at −78° C. for 10 min, and then at room temperature for a further 1.5 h. The mixture was poured into water (100 mL) and extracted with dichloromethane (1×100 mL, 2×50 mL). The extracts were dried (MgSO₄), filtered and concentrated to give a brown oil. The oil was purified by reverse-phase column chromatography (Biotage Ultra Cis cartridge, 120 g, 25), eluting with a gradient of 70% acetonitrile in water for 2 column volumes, 70-95% acetonitrile in water over 5 column volumes and finally 95% acetonitrile in water for 5 column volumes. Fractions containing the product were pooled and concentrated under reduced pressure to give 6-fluoro-1-(triisopropylsilyl)-1H-indole (4.173 g, 43%), as a light-yellow oil. LC-MS (System 2, 8 min buffered method); RT=4.53 min, [M−H]⁻291. ¹H NMR (400 MHz, CDCl₃, ppm) δ 7.52 (dd, J9.1, 5.8 Hz, 1H), 7.22 (d, J3.0 Hz, 1H), 7.19 (dd, J11.2, 2.1 Hz, 1H), 6.88 (td, J9.1, 2.1 Hz, 1H), 6.59 (d, J3.0 Hz, 1H), 1.68 (sept, J7.3 Hz, 3H), 1.14 (d, J7.3 Hz, 18H). ¹⁹F NMR (373 MHz, CDCl₃, ppm) 6-121.65 (dt, J9.1, 5.8 Hz).

Synthesis of 6-fluoro-5-iodo-1-(triisopropylsilyl)-1H-indole. 6-Fluoro-1-(triisopropylsilyl)-1H-indole (4.173 g, 14.3 mmol) and N,N,N′,N″,N″-pentamethyldiethylenetriamine (3.80 mL, 18.2 mmol) were dissolved in anhydrous tetrahydrofuran (25 mL) and cooled to −78° C. under nitrogen. A solution of sec-butyllithium (1.27 M in cyclohexane, 18.0 mL, 22.9 mmol) was added and the mixture stirred at −78° C. for 6 h. A solution of iodine (5.74 g, 22.6 mmol) in anhydrous tetrahydrofuran (12 mL) was added and the mixture stirred at −78° C. for 30 min. The mixture was poured into water (100 mL) and extracted with dichloromethane (1×100 mL, 2×50 mL). The combined extracts were washed with aqueous sodium thiosulfate (10%, 80 mL), dried (MgSO₄), filtered and concentrated to give a brown oil. The oil as purified by reversed-phase column chromatography (Biotage Ultra Cis cartridge, 120 g, 25), eluting with a gradient of 70% acetonitrile in water for 2 column volumes, 70-95% acetonitrile in water over 10 column volumes, and finally 95% acetonitrile in water for 5 column volumes. Fractions containing the product were pooled and concentrated under reduced pressure to give 6-fluoro-5-iodo-1-(triisopropylsilyl)-1H-indole (4.68 g, 78%), as a light brown oil. LC-MS (System 2, 8 min buffered); RT=4.88 min, [M−Si[CH(CH₃)_{2]3}]⁻260. ¹H NMR (400 MHz, CDCl₃, ppm) δ 7.94 (d, J6.1 Hz, 1H), 7.24 (d, J10.3 Hz, 1H), 7.21 (d, J2.8 Hz, 1H), 6.53 (d, J2.8 Hz, 1H), 1.66 (sept, J7.3 Hz, 3H), 1.13 (d, J7.3 Hz, 18H). ¹⁹F NMR (373 MHz, CDCl₃, ppm) 6-102.90 (dd, J10.3, 6.1 Hz).

Synthesis of tert-butyl 2-(6-fluoro-1-(triisopropylsilyl)-1H-indol-5-yl)acetate. Zinc dust (2.97 g, 45.4 mmol) was suspended in anhydrous tetrahydrofuran (80 mL) under nitrogen and chlorotrimethylsilane (0.45 mL, 3.6 mmol) was added. The mixture was heated at 65° C. for 15 min and then cooled to 50° C. over 20 min. Tert-butyl bromoacetate (4.1 mL, 27.8 mmol) was added dropwise over 5 min and the mixture was stirred at 50° C. for 40 min to generate (2-(tert-butoxy)-2-oxoethyl)zinc (II) bromide. The mixture was then allowed to cool and the solids to settle over 20 min. Separately a mixture of 6-fluoro-5-iodo-1-(triisopropylsilyl)-1H-indole (4.43 g, 10.6 mmol), 2-dicyclohexylphosphino-2′,4′,6′-triisopropylbiphenyl (1.147 g, 2.4 mmol) and tris(dibenzylideneacetone)dipalladium (0) (1.134 g, 1.20 mmol) in anhydrous tetrahydrofuran (10 mL) was stirred under nitrogen at room temperature for 10 min. The supernatant of the solution containing (2-(tert-butoxy)-2-oxoethyl)zinc (II) bromide was withdrawn by syringe and added to the mixture of the indole and palladium catalyst. The mixture was then heated at reflux for 4.5 h, cooled to room temperature and poured into saturated aqueous ammonium chloride (200 mL). The mixture was extracted with ethyl acetate (200 mL), which was washed with brine (200 mL), dried (MgSO₄), filtered and concentrated under reduced pressure to give a dark orange oil (10.22 g). The oil was adsorbed onto silica gel (30 g) using dichloromethane (80 mL) and purified by flash column chromatography (SiliCycle SiliaSep cartridge, 220 g) eluting with 0% ethyl acetate in heptane for 2 column volumes, and 0-50% ethyl acetate in heptane over 15 column volumes. Fractions containing the product were pooled, concentrated under reduced pressure and the residue re-purified by reverse-phase column chromatography (Biotage Ultra Cis cartridge, 120 g, 25), eluting with a gradient of 70% acetonitrile in water for 2 column volumes, 70-95% acetonitrile in water over 10 column volumes and finally 95% acetonitrile in water for 2 column volumes. Fractions containing the product were pooled and concentrated under reduced pressure to give tert-butyl 2-(6-fluoro-1-(triisopropylsilyl)-1H-indol-5-yl)acetate (3.418 g, 79%), as a pale-yellow oil. LCMS (System 2, 8 min buffered method); RT=4.71 min, [M+H]⁺ 406. ¹H NMR (400 MHz, CDCl₃, ppm) δ 7.42 (d, J8.0 Hz, 1H), 7.18 (d, J3.2 Hz, 1H), 7.18 (d, J11.6 Hz, 1H), 6.54 (d, J3.2 Hz, 1H), 3.64 (s, 2H), 1.72-1.61 (m, 3H), 1.46 (s, 9H), 1.13 (d, J 7.2 Hz, 18H). ¹⁹F NMR (373 MHz, CDCl₃, ppm) 6-124.8 (m).

Synthesis of tert-butyl 2-(3-chloro-6-fluoro-1-(triisopropylsilyl)-1H-indol-5-yl)acetate. N-Chlorosuccinimide (1.250 g, 9.36 mmol) and tert-butyl 2-(6-fluoro-1-(triisopropylsilyl)-1H-indol-5-yl)acetate (3.418 g, 8.43 mmol) were mixed together in anhydrous N,N-dimethylformamide (50 mL) and stirred at room temperature under nitrogen for 16 h. The mixture was diluted with ethyl acetate (200 mL) and washed with aqueous sodium bisulfate (5%, 150 mL), saturated aqueous sodium bicarbonate (2×100 mL) and brine (100 mL). The organic phase was dried (MgSO₄), filtered and concentrated under reduced pressure to give a yellow oil (3.44 g). The oil was adsorbed onto silica gel (9 g) using dichloromethane (50 mL) and purified by flash column chromatography (SiliCycle SiliaSep cartridge, 220 g) eluting with 0% ethyl acetate in heptane for 2 column volumes, 0-20% ethyl acetate in heptane over 10 column volumes, and finally 20% ethyl acetate in heptane for 2 column volumes. Fractions containing the product were pooled and concentrated under reduced pressure to give tert-butyl 2-(3-chloro-6-fluoro-1-(triisopropylsilyl)-1H-indol-5-yl)acetate (2.561 g, 69%), as a yellow oil. LC-MS (System 2, 8 min buffered method); RT=4.90 min, [M+NH_{4]}⁺457/459. ¹H NMR (400 MHz, CDCl₃, ppm) δ 7.42 (d, J8.0 Hz, 1H), 7.15 (d, J11.6 Hz, 1H), 7.13 (s, 1H), 3.67 (s, 2H), 1.69-1.57 (m, 3H), 1.47 (s, 9H), 1.13 (d, J7.2 Hz, 18H). ¹⁹F NMR (373 MHz, CDCl₃, ppm) 6-122.5 (m).

Synthesis of tert-butyl 2-(3-chloro-6-fluoro-1H-indol-5-yl)acetate. Tert-butyl 2-(3-chloro-6-fluoro-1-(triisopropylsilyl)-1H-indol-5-yl)acetate (2.561 g, 5.82 mmol) was dissolved in anhydrous tetrahydrofuran (20 mL) under nitrogen and cooled to 0° C. To this was added a solution of tetrabutylammonium fluoride (1M in THF, 12.0 mL, 12.0 mmol) over 5 min and the mixture was stirred for 75 min. The mixture was diluted with ethyl acetate (200 mL) and washed with H₂O (2×150 mL) and brine (150 mL). The organic phase was dried (MgSO₄), filtered and concentrated under reduced pressure to give a pale orange oil (2.62 g). The oil was adsorbed onto silica gel (16 g) using dichloromethane (50 mL) and purified by flash column chromatography (SiliCycle SiliaSep cartridge, 120 g) eluting with 0% ethyl acetate in heptane for 2 column volumes and 0-30% ethyl acetate in heptane over 10 column volumes. Fractions containing the product were pooled and concentrated under reduced pressure to give tert-butyl 2-(3-chloro-6-fluoro-1H-indol-5-yl)acetate (1.508 g, 91%), as a pale pink solid. LC-MS (System 2, 4.5 min buffered); RT=3.52 min, [M−H]⁻482/484. ¹H NMR (400 MHz, CDCl₃, ppm) δ 8.09 (br s, 1H), 7.44 (d, J7.2 Hz, 1H), 7.10 (d, J2.8 Hz, 1H), 7.03 (d, J10.0 Hz, 1H), 3.68 (d, J1.2 Hz, 2H), 1.47 (s, 9H). ¹⁹F NMR (373 MHz, CDCl₃, ppm) 6-122.6 (m).

Synthesis of 2-(3-chloro-6-fluoro-1H-indol-5-yl)acetic acid. A mixture of tert-butyl 2-(3-chloro-6-fluoro-1H-indol-5-yl)acetate (1.508 g, 5.31 mmol) and sodium hydroxide (0.650 g, 16.3 mmol) in methanol (40 mL) and water (4 mL) was stirred at 65° C. for 5.5 h, under nitrogen. The mixture was then concentrated under reduced pressure to give a pale-red solid. Two other impure batches prepared previously (0.147 g and 0.048 g respectively) were also added. The combined crude batches were purified by reverse-phase column chromatography (Biotage Ultra Cis cartridge, 60 g, 25), eluting with a gradient of 20% acetonitrile in water+0.1% formic acid for 1 column volume and 20-70% acetonitrile in water+0.1% formic acid over 15 column volumes. Fractions containing the product were pooled, concentrated under reduced pressure and the residue re-purified by reverse-phase column chromatography (Biotage Ultra Cis cartidge, 60 g, 25), eluting with a gradient of 5% acetonitrile in water+0.1% formic acid for 2 column volumes, 5-30% acetonitrile in water+0.1% formic acid over 10 column volumes and finally₃₀-7₀% acetonitrile in water+0.1% formic acid over 5 column volumes. Fractions containing the product were pooled and concentrated under reduced pressure to give 2-(3-chloro-6-fluoro-1H-indol-5-yl)acetic acid (0.824 g, 56%), as a pale pink solid. L^{C}-MS (System 2, 4.5 min buffered); RT=1.74 min, [M−H−CO₂]⁻182/184. ¹H NMR (400 MHz, (CD₃)₂SO, ppm) δ 12.33 (s, 1H, exchanges with deuterium over time), 11.37 (s, 1H), 7.49 (d, J2.4 Hz, 1H), 7.41 (d, J7.6 Hz, 1H), 7.20 (d, J10.8 Hz, 1H), 3.61 (s, 2H). ¹⁹F NMR (373 MHz, (CD₃)₂SO, ppm) δ−123.8 (m).

### Synthesis of 2-(3-chloro-1H-pyrrolo[2,3-b]pyridin-5-yl)acetic acid

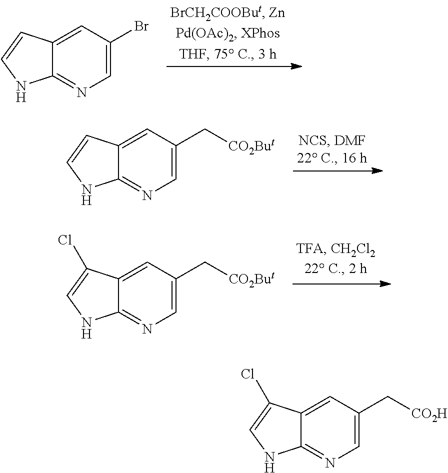

Synthesis of tert-butyl 2-(1H-pyrrolo[2,3-b]pyridin-5-yl)acetate. Zinc dust (1.244 g, 19.00 mmol) was suspended in anhydrous tetrahydrofuran (40 mL) under nitrogen and chlorotrimethylsilane (0.16 mL, 1.3 mmol) was added. The mixture was heated at reflux for 5 min, followed by sonication for 1 min and then heated at 65° C. for a further 5 min. Tert-butyl bromoacetate (1.87 mL, 12.7 mmol) was added dropwise over 10 min and the mixture was stirred at 50° C. for 45 min to generate (2-(tert-butoxy)-2-oxoethyl)zinc (II) bromide. The mixture was then allowed to cool and the excess of zinc to settle over 30 min. Separately a mixture of 5-bromo-7-azaindole (1.000 g, 5.100 mmol), 2-dicyclohexylphosphino-2′,4′,6′-triisopropylbiphenyl (0.484 g, 1.00 mmol) and palladium (II) acetate (0.057 g, 0.30 mmol) was prepared under nitrogen at room temperature. The supernatant of the solution containing (2-(tert-butoxy)-2-oxoethyl) zinc(II) bromide was added and the mixture then heated at reflux for 3 h. The mixture was cooled to room temperature, poured into saturated aqueous ammonium chloride (50 mL), and the organic solvent was removed under reduced pressure. Ethyl acetate (100 mL) was added, the layers were separated, and the organic phase was washed with saturated aqueous ammonium chloride (50 mL) and brine (50 mL). The organic phase was dried (MgSO₄), filtered and concentrated under reduced pressure to give a yellow solid. The solid was adsorbed onto silica gel using dichloromethane and purified by flash column chromatography (40 g) eluting with 20-40% ethyl acetate in heptane. Fractions containing the product were pooled, concentrated under reduced pressure to give tert-butyl 2-(1H-pyrrolo[2,3-b]pyridin-5-yl)acetate (0.868 g, 74%), as a colourless solid. LC-MS [System 2, 4.5 min buffered method] RT=2.75 min; [M+H]⁺=233. ¹H NMR (400 MHz, CDCl₃, ppm) δ 9.76 (br s, 1H), 8.21 (s, 1H), 8.00 (d, J2 Hz, 1H), 7.37 (d, J4 Hz, 1H), 6.54 (d, J4 Hz), 3.65 (s, 2H), 1.45 (s, 9H).

Synthesis of tert-butyl 2-(3-chloro-1H-pyrrolo[2,3-b]pyridin-5-yl)acetate. N-Chlorosuccinimide (0.549 g, 4.11 mmol) and tert-butyl 2-(1H-pyrrolo[2,3-b]pyridin-5-yl)acetate (0.868 g, 3.74 mmol) were mixed together in anhydrous N,N-dimethylformamide (6 mL) and stirred at room temperature under nitrogen for 16 h. The mixture was diluted with ethyl acetate (50 mL) and washed with aqueous sodium bisulfate (10%, 50 mL), saturated aqueous sodium bicarbonate (2×50 mL) and brine (50 mL). The organic phase was dried (MgSO₄), filtered and concentrated under reduced pressure. The residue was adsorbed onto silica gel using dichloromethane and purified by flash column chromatography (40 g), eluting with 20-40% ethyl acetate in heptane. Fractions containing the product were pooled and concentrated under reduced pressure to give tert-butyl 2-(3-chloro-1H-pyrrolo[2,3-b]pyridin-5-yl)acetate (0.621 g, 62%), as a colourless solid. LC-MS [System 2, 4.5 min buffered method] RT=3.08 min; [M+H]⁺=267/269. ¹H NMR (400 MHz, CDCl₃, ppm) δ 10.18 (br s, 1H), 8.26 (d, J2 Hz, 1H), 8.07-8.03 (m, 1H), 7.37-7.35 (m, 1H), 3.69 (s, 2H), 1.46 (s, 9H).

Synthesis of 2-(3-chloro-1H-pyrrolo[2,3-b]pyridin-5-yl)acetic acid. Trifluoroacetic acid (8.00 mL, 104 mmol) was added to a solution of tert-butyl 2-(3-chloro-1H-pyrrolo[2,3-b]pyridin-5-yl)acetate (0.852 g, 3.20 mmol) in dichloromethane (4 mL). The mixture was stirred at room temperature for 2 h. The mixture was concentrated under reduced pressure and the residue treated with triethylamine:acetonitrile (10 mL, 1:9). The mixture was concentrated under reduced pressure and the purple residue treated with formic acid:acetonitrile (10 mL, 1:9). The mixture was again concentrated under reduced pressure and the oil dissolved in dimethylsulfoxide and purified by reverse-phase column chromatography (Biotage Ultra Cis column, 60 g, 25), eluting with a gradient 20-90% acetonitrile in water+0.1% formic acid over 10 column volumes. Fractions containing the product were pooled and concentrated under reduced pressure to give 2-(3-chloro-1H-pyrrolo[2,3-b]pyridin-5-yl)acetic acid (0.540 g, 80%), as a light purple solid. LC-MS (System 2, 4.5 min buffered method); RT=1.49 min, [M+H]⁺ 211/213. ¹H NMR (400 MHz, (CD₃)₂SO, ppm) δ 12.40 (s, 1H), 11.92 (s, 1H), 8.19 (d, J2 Hz, 1H), 7.81 (d, J2 Hz, 1H), 7.66 (d, J3 Hz, 1H), 3.73 (s, 2H).

### Synthesis of 2-((2R,4S)-4-((tert-butyldimethylsilyl)oxy)pyrrolidin-2-yl)-6-cyclopropylimidazo[1,2-a]pyridine

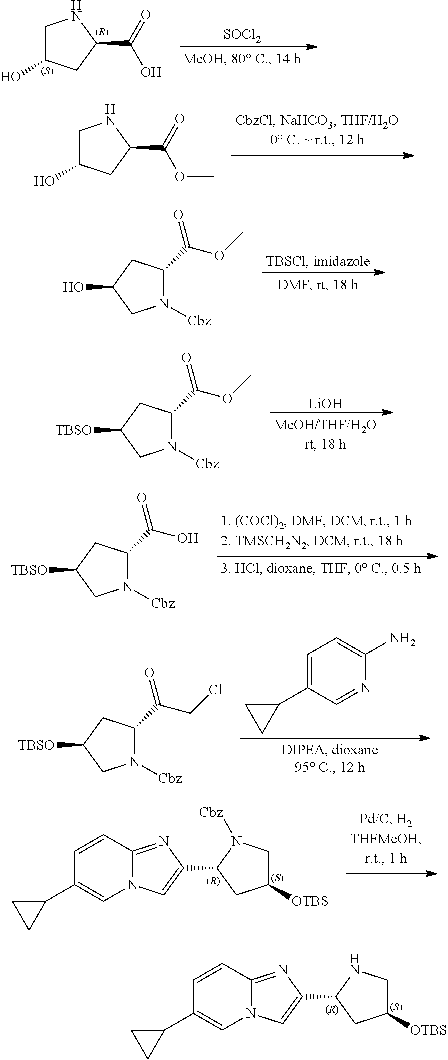

Synthesis of methyl (2R,4S)-4-hydroxypyrrolidine-2-carboxylate. To the mixture of (2R,4S)-4-hydroxypyrrolidine-2-carboxylic acid (5 g, 38.2 mmol) in MeOH (50 mL) was added SOCl₂ (5 mL) at 0° C. The reaction mixture was stirred at 80° C. for 14 h. After cooled to room temperature, the reaction was concentrated to afford methyl (2R,4S)-4-hydroxypyrrolidine-2-carboxylate (6.5 g, crude) as a white solid, which was used in next step directly without further purification. ESI-MS [M+H]⁺: 146.1.

Synthesis of 1-benzyl 2-methyl (2R,4S)-4-hydroxypyrrolidine-1,2-dicarboxylate. To the mixture of methyl (2R,4S)-4-hydroxypyrrolidine-2-carboxylate (6.5 g, crude) and NaHCO₃ (9.6 g, 114.5 mmol) in THF/H₂O (150 mL/150 mL) was added CbzCl (9.7 g, 57.3 mmol) at 0° C. The mixture was then warmed to room temperature and stirred for 12 h. The reaction was concentrated and aqueous layer was extracted with EtOAc (100 mL×3). The combined organic layers were washed with brine (100 mL), dried over Na₂SO₄ and concentrated. The residue was purified by flash column chromatography (EA/PE from 0 to 100%) to afford 1-benzyl 2-methyl (2R,4S)-4-hydroxypyrrolidine-1,2-dicarboxylate (5.8 g, 54% yield for two steps) as a white solid. ESI-MS [M+H]⁺: 280.1.

Synthesis of 1-benzyl 2-methyl (2R,4S)-4-((tert-butyldimethylsilyl)oxy)pyrrolidine-1,2-dicarboxylate. To a mixture of 1-benzyl 2-methyl (2R,4S)-4-hydroxypyrrolidine-1,2-dicarboxylate (5.8 g, 20.8 mmol) and Imidazole (4.2 g, 62.4 mmol) in DMF (50 mL) was added TBSCl (4.7 g, 31.2 mmol) and the mixture was stirred at room temperature for 18 h. Water (50 mL) was added and extracted with EtOAc (100 mL×3). The combined organic layers were washed with brine (100 mL), dried over Na₂SO₄ and concentrated. The residue was purified by silica gel column chromatography (EtOAc/PE from 0 to 15%) to afford 1-benzyl 2-methyl (2R,3S,4R)-3,4-bis((tert-butyldimethylsilyl)oxy)pyrrolidine-1,2-dicarboxylate (6 g, 73% yield) as a white solid. ESI-MS [M+H]+: 394.2.

Synthesis of (2R,4S)-1-((benzyloxy)carbonyl)-4-((tert-butyldimethylsilyl)oxy)pyrrolidine-2-carboxylic acid. A mixture of 1-benzyl 2-methyl (2R,4S)-4-((teroom temperature-butyldimethylsilyl)oxy)pyrrolidine-1,2-dicarboxylate (4.0 g, 10.2 mmol) and LiOH·H₂O (0.85 g, 20.4 mmol) in MeOH/THF/H₂O (30 mL/30 mL/10 mL) was stirred at room temperature for 18 h. The reaction was concentrated, pH of the residue was adjusted to ˜5 by HCl (aq. 2M) and extracted with iPrOH/CHCl₃ (1/3, 50 mL×3). The combined organic layers were washed with brine (50 mL), dried over Na₂SO₄ and concentrated to afford (2R,4S)-1-((benzyloxy)carbonyl)-4-((tert-butyldimethylsilyl)oxy)pyrrolidine-2-carboxylic acid (3.4 g, crude) as a colorless oil, which was used in next step directly. ESI-MS [M+H]+: 380.2.

Synthesis of benzyl (2R,4S)-4-((tert-butyldimethylsilyl)oxy)-2-(2-chloroacetyl)pyrrolidine-1-carboxylate. To a solution of (2R,4S)-1-((benzyloxy)carbonyl)-4-((tert-butyldimethylsilyl)oxy)pyrrolidine-2-carboxylic acid (400 mg, 1.05 mmol) in DCM (10 mL) was added oxalyl chloride (162 mg, 1.27 mmol) and a drop of DMF at 0° C. and the mixture was stirred at room temperature for 1 h. The reaction was concentrated, the residue was re-dissolved in DCM (10 mL). TMSCH₂N₂ (1.06 mL, 2M in hexane, 2.12 mmol) was added at 0° C. and the resulting mixture was stirred at room temperature for 18 h. The reaction mixture was concentrated to remove the solvent and the residue was re-dissolved in THF (10 mL), HCl (0.32 mL, 4M in dioxane, 1.27 mmol) was added at 0° C. After the mixture was stirred at 0° C. for 0.5 h, the reaction was concentrated to give the crude, which was purified by Prep-TLC (PE/EtOAc=20%) to afford benzyl (2R,4S)-4-((tert-butyldimethylsilyl)oxy)-2-(2-chloroacetyl)pyrrolidine-1-carboxylate (170 mg, 39% yield) as a yellow solid. ESI-MS [M+H]+: 412.2.

Synthesis of benzyl (2R,4S)-4-((tert-butyldimethylsilyl)oxy)-2-(6-cyclopropylimidazo[1,2-a]pyridin-2-yl)pyrrolidine-1-carboxylate. A mixture of benzyl (2R,4S)-4-((tert-butyldimethylsilyl)oxy)-2-(2-chloroacetyl)pyrrolidine-1-carboxylate (250 mg, 0.61 mmol), 5-cyclopropylpyridin-2-amine (165 mg, 1.22 mmol) and DIPEA (235 mg, 1.82 mmol) in 1,4-dioxane (5 mL) was stirred at 95° C. for 12 h. The reaction was quenched with water (30 mL) and extracted with EtOAc (30 mL×3). The combined organic layers were washed with brine (30 mL), dried over Na₂SO₄ and concentrated. The residue was purified by Prep-TLC (MeOH/DCM=10%) to afford benzyl (2R,4S)-4-((tert-butyldimethylsilyl)oxy)-2-(6-cyclopropylimidazo[1,2-a]pyridin-2-yl)pyrrolidine-1-carboxylate (120 mg, 40% yield) as a yellow oil. ESI-MS [M+H]+: 492.3.

Synthesis of 2-((2R,4S)-4-((tert-butyldimethylsilyl)oxy)pyrrolidin-2-yl)-6-cyclopropylimidazo[1,2-a]pyridine. A mixture of benzyl (2R,4S)-4-((tert-butyldimethylsilyl)oxy)-2-(6-cyclopropylimidazo[1,2-a]pyridin-2-yl)pyrrolidine-1-carboxylate (460 mg, 0.95 mmol) and Pd/C (460 mg) in THF/MeOH (5 mL/5 mL) was stirred at room temperature under H₂ for 2 h. The reaction was filtered and washed with MeOH (30 mL). The filtrate was concentrated to afford crude 2-((2R,4S)-4-((tert-butyldimethylsilyl)oxy)pyrrolidin-2-yl)-6-cyclopropylimidazo[1,2-a]pyridine (360 mg, crude) as brown oil, which was used in the next step directly. ESI-MS [M+H]+: 358.2.

### Example 1

### Synthesis of 2-(7-chloroimidazo[1,2-a]pyridin-1-yl)-N-(6-(((6-cyclopropylimidazo[1,2-a]pyridin-2-yl)methyl)amino)pyrimidin-4-yl)acetamide (I-1)

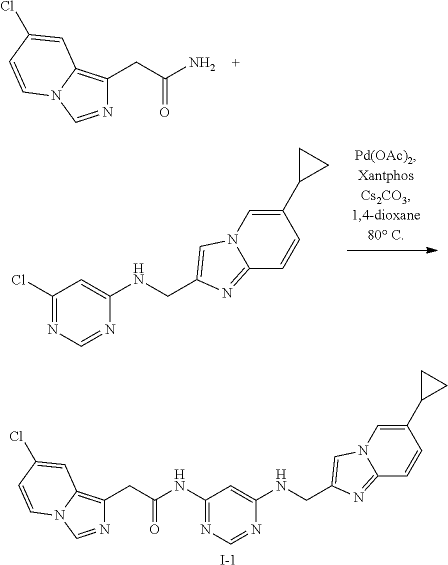 [SMILES: NC(=O)Cc1ncn2ccc(Cl)cc12 ; Clc1cc(NCc2cn3cc(C4CC4)ccc3n2)ncn1 ; O=C(Cc1ncn2ccc(Cl)cc12)Nc1cc(NCc2cn3cc(C4CC4)ccc3n2)ncn1 ; [I-]]

A suspension of 6-chloro-N-((6-cyclopropylimidazo[1,2-a]pyridin-2-yl)methyl)pyrimidin-4-amine (550 mg 1.83 mmol), 2-(7-chloroimidazo[1,5-a]pyridin-1-yl)acetamide (385 mg, 1.83 mmol), Pd(OAc)₂ (82 mg, 0.37 mmol), Xantphos (425 mg, 0.73 mmol) and Cs₂CO₃ (897 mg, 2.75 mmol) in 1,4-dioxane (20 mL) was degassed with N₂ for 10 minutes then heated to 80° C. for 6 h. The mixture was cooled to room temperature, diluted with water (100 mL) and then extracted with EtOAc (3×50 mL). The combined organics were dried over MgSO₄ then concentrated in vacuo. The residue was purified by reverse phase preparative HPLC to give the title compound (78 mg, 90%). ESI-MS (M+H⁺): 473.2, ¹H NMR (400 MHz, DMSO) δ10.42 (s, 1H), 8.39 (s, 1H), 8.35-8.26 (m, 3H), 8.20 (s, 1H), 7.82 (m, 2H), 7.59 (s, 1H), 7.35 (d, J=9.3 Hz, 1H), 7.23 (s, 1H), 6.97-6.93 (3, 1H), 6.65 (dd, J=1.8, 7.3 Hz, 1H), 4.55 (br s, 2H), 3.94 (s, 2H), 1.94-1.86 (m, 1H), 0.93-0.86 (m, 2H), 0.68-0.62 (in, 2H).

Using a similar procedure to that used for 2-(7-chloroimidazo[1,5-a]pyridin-1-yl)-N-(6-(((6-cyclopropylimidazo[1,2-a]pyridin-2-yl)methyl)amino)pyrimidin-4-yl)acetamide (Example 1), the compounds in the table below were prepared using 6-chloro-N-((6-cyclopropylimidazo[1,2-a]pyridin-2-yl)methyl)pyrimidin-4-amine and an appropriate coupling partner followed by purification by preparative HPLC.

| | | Coupling | |
|---|---|---|---|
| EG | Compound | Partner | Analytical Data |
| 2 | 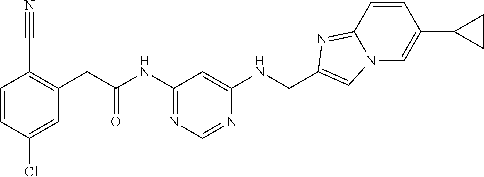 [SMILES: N#Cc1ccc(Cl)cc1CC(=O)Nc1cc(NCc2cn3cc(C4CC4)ccc3n2)ncn1] 2-(5-chloro-2-cyanophenyl)-N-(6-(((6-cyclopropylimidazo[1,2- a]pyridin-2-yl)methyl)amino)pyrimidin-4-yl)acetamide (I-2) | 2-(5- chloro-2- cyanophenyl) acetamide | ESI-MS (M + H)⁺: 458.3, ¹H NMR (400 MHz, DMSO) δ 10.71 (s, 1H), 8.30 (s, 1H), 8.25 (s, 1H), 7.91-7.85 (m, 2H), 7.70 (d, J = 2.6 Hz, 1H), 7.62- 7.58 (m, 2H), 7.37 (d, J = 8.4 Hz, 1H), 7.20 (s, 1H), 6.96 (dd, J = 2.1, 9.7 Hz, 1H), 4.58 (s, 2H), 4.03 (s, 2H), 1.95- 1.87 (m, 1H), 0.94-0.89 (m, 2H), 0.69-0.65 (m, 2H). |
| 3 | 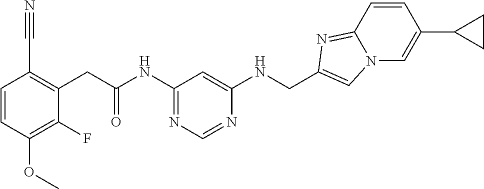 [SMILES: COc1ccc(C#N)c(CC(=O)Nc2cc(NCc3cn4cc(C5CC5)ccc4n3)ncn2)c1F] 2-(6-cyano-2-fluoro-3-methoxyphenyl)-N-(6-(((6- cyclopropylimidazo[1,2-a]pyridin-2-yl)methyl)amino) pyrimidin-4-yl)acetamide (I-3) | 2-(6- cyano-2- fluoro-3- methoxy- phenyl) acetamide | ESI-MS (M + H)⁺: 472.3, ¹H NMR (400 MHz, DMSO) δ 10.75 (s, 1H), 8.30 (s, 1H), 8.24 (s, 1H), 7.87 (s, 1H), 7.71 (dd, J = 1.3, 8.9 Hz, 1H), 7.61 (s, 1H), 7.37 (d, J = 9.7 Hz, 1H), 7.31 (t, J = 8.6 Hz, 1H), 7.20 (s, 1H), 6.96 (dd, J = 1.8, 9.4 Hz, 1H), 4.58 (s, 2H), 4.00 (s, 2H), 3.95 (s, 3H), 1.95-1.87 (m, 1H), 0.94-0.89 (m, 2H), 0.69-0.64 (m, 2H). |
| 4 | 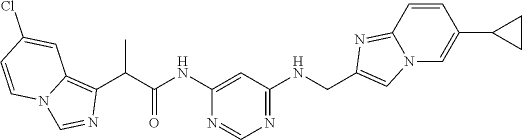 [SMILES: CC(C(=O)Nc1cc(NCc2cn3cc(C4CC4)ccc3n2)ncn1)c1ncn2ccc(Cl)cc12] 2-(7-chloroimidazo[1,5-a]pyridin-1-yl)-N-(6-(((6- cyclopropylimidazo[1,2-a]pyridin-2-yl)methyl)amino) pyrimidin-4-yl)propanamide (I-4) | 2-(7- chloro- imidazo [1,5-a] pyridin- 1-yl) propanamide | ESI-MS (M + H)⁺: 487.2, ¹H NMR (400 MHz, DMSO) δ 10.32 (s, 1H), 8.35-8.31 (m, 2H), 8.29 (s, 1H), 8.18 (s, 1H), 7.94 (s, 2H), 7.59 (s, 1H), 7.37 (d, J = 9.3 Hz, 1H), 7.25 (s, 1H), 6.96 (dd, J = 1.8, 9.3 Hz, 1H), 6.67 (dd, J = 2.1, 7.5 Hz, 1H), 4.57-4.56 (m, 2H), 4.29 (q, J = 7.0 Hz, 1H), 1.95- 1.87 (m, 1H), 1.48 (d, J = 7.0 Hz, 3H), 0.94-0.88 (m, 2H), 0.69-0.63 (m, 2H). |
| 5 | 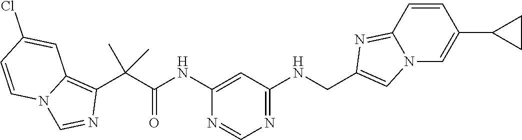 [SMILES: CC(C)(C(=O)Nc1cc(NCc2cn3cc(C4CC4)ccc3n2)ncn1)c1ncn2ccc(Cl)cc12] 2-(7-chloroimidazo[1,5-a]pyridin-1-yl)-N-(6-(((6-cyclopropylimidazo[1,2- a]pyridin-2-yl)methyl)amino)pyrimidin-4-yl)-2-methylpropanamide (I-5) | 2-(7- chloro- imidazo [1,5-a] pyridin- 1-yl)-2- methyl propanamide | ESI-MS (M + H)⁺: 501.4, ¹H NMR (400 MHz, DMSO) δ 9.39 (s, 1H), 8.47 (s, 1H), 8.37 (d, J = 7.5 Hz, 1H), 8.30 (s, 1H), 8.13 (s, 1H), 7.90 (s, 1H), 7.60 (d, J = 7.0 Hz, 2H), 7.38 (d, J = 9.3 Hz, 1H), 7.28 (s, 1H), 6.97 (dd, J = 1.8, 9.3 Hz, 1H), 6.70 (dd, J = 2.0, 7.4 Hz, 1H), 4.57-4.57 (m, 2H), 1.95-1.87 (m, 1H), 1.69 (s, 6H), 0.94-0.89 (m, 2H), 0.69-0.64 (m, 2H). |
| 6 | 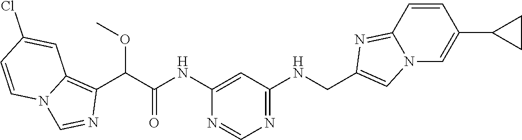 [SMILES: COC(C(=O)Nc1cc(NCc2cn3cc(C4CC4)ccc3n2)ncn1)c1ncn2ccc(Cl)cc12] 2-(7-chloroimidazo[1,5-a]pyridin-1-yl)-N-(6-(((6- cyclopropylimidazo[1,2-a]pyridin-2-yl)methyl)amino)pyrimidin-4- yl)-2-methoxyacetamide (I-6) | 2-(7- chloro- imidazo [1,5-a] pyridin- 1-yl)-2- methoxy acetamide | ¹H NMR (400 MHz, DMSO) δ 9.86 (s, 1H), 8.39 (dd, J = 3.5, 3.5 Hz, 2H), 8.29 (s, 1H), 8.22 (s, 1H), 7.96 (s, 1H), 7.92 (s, 1H), 7.62 (s, 1H), 7.37 (d, J = 9.3 Hz, 1H), 7.28 (s, 1H), 6.96 (dd, J = 1.8, 9.3 Hz, 1H), 6.77 (dd, J = 2.1, 7.5 Hz, 1H), 5.37 (s, 1H), 4.59-4.59 (m, 2H), 3.30 (s, 3H), 1.95-1.87 (m, 1H), 0.95-0.88 (m, 2H), 0.70-0.63 (m, 2H). |
| 7 | 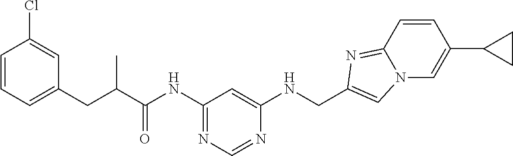 [SMILES: CC(Cc1cccc(Cl)c1)C(=O)Nc1cc(NCc2cn3cc(C4CC4)ccc3n2)ncn1] 3-(3-chlorophenyl)-N-(6-(((6-cyclopropylimidazo[1,2-a]pyridin-2- yl)methyl)amino)pyrimidin-4-yl)-2-methylpropanamide (I-7) | 3-(3-chloro phenyl)-2- methyl- propanamide | ESI-MS (M + H)⁺: 461.4, ¹H NMR (400 MHz, DMSO) δ 10.32 (s, 1H), 8.30 (s, 1H), 8.19 (s, 1H), 7.86 (s, 1H), 7.35-7.23 (m, 4H), 7.18 (d, J = 7.4 Hz, 1H), 6.97 (dd, J = 1.8, 9.4 Hz, 1H), 4.59 (s, 2H), 2.99-2.90 (m, 2H), 2.64- 2.56 (m, 1H), 1.96-1.88 (m, 1H), 1.05 (d, J = 6.1 Hz, 3H), 0.95-0.89 (m, 2H), 0.69-0.64 (m, 2H) |
| 8 | 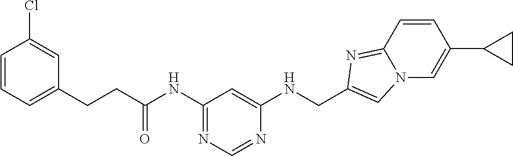 [SMILES: O=C(CCc1cccc(Cl)c1)Nc1cc(NCc2cn3cc(C4CC4)ccc3n2)ncn1] 3-(3-chlorophenyl)-N-(6-(((6-cyclopropylimidazo[1,2-a] pyridin-2-yl)methyl)amino)pyrimidin-4-yl)propanamide (I-8) | 3-(3- chloro- phenyl) propanamide | ESI-MS (M + H)⁺: 447.4, ¹H NMR (400 MHz, DMSO) δ 10.31 (s, 1H), 8.30 (s, 1H), 8.20 (s, 1H), 7.86 (s, 1H), 7.62 (s, 1H), 7.38 (d, J = 9.3 Hz, 1H), 7.35-7.19 (m, 5H), 6.97 (dd, J = 1.8, 9.4 Hz, 1H), 4.58-4.54 (m, 2H), 2.92- 2.86 (m, 2H), 2.73-2.67 (m, 2H), 1.96-1.88 (m, 1H), 0.95-0.89 (m, 2H), 0.69-0.65 (m, 2H) |
| 9 | 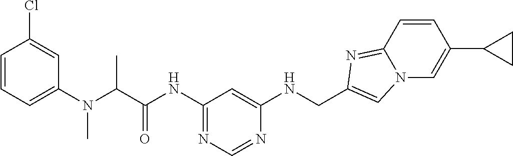 [SMILES: CC(C(=O)Nc1cc(NCc2cn3cc(C4CC4)ccc3n2)ncn1)N(C)c1cccc(Cl)c1] 2-((3-chlorophenyl)(methyl)amino)-N-(6-(((6-cyclopropyl imidazo[1,2-a]pyridin-2-yl)methyl)amino)pyrimidin-4-yl)propanamide (I-9) | 2-((3- chloro- phenyl) (methyl) amino) propanamide | ESI-MS (M + H)⁺: 476.4, ¹H NMR (400 MHz, DMSO) δ 10.30-10.27 (m, 1H), 8.28-8.27 (m, 1H), 8.20 (s, 1H), 7.87 (br s, 1H), 7.59 (s, 1H), 7.36 (d, J = 9.3 Hz, 1H), 7.25 (s, 1H), 7.19 (dd, J = 8.2, 8.2 Hz, 1H), 6.95 (dd, J = 1.7, 9.4 Hz, 1H), 6.86-6.83 (m, 1H), 6.78-6.74 (m, 1H), 6.72-6.68 (m, 1H), 4.72 (q, J = 6.9 Hz, 1H), 4.56 (br s, 2H), 2.88 (s, 3H), 1.94-1.86 (m, 1H), 1.36 (d, J = 7.0 Hz, 3H), 0.93- 0.87 (m, 2H), 0.67-0.62 (m, 2H). |

### Example 10

### Synthesis of 2-(5-chloro-2-(1H-tetrazol-5-yl)phenyl)-N-(6-(((6-cyclopropylimidazo[1,2-a]pyridin-2-yl)methyl)amino)pyrimidin-4-yl)acetamide (I-10)

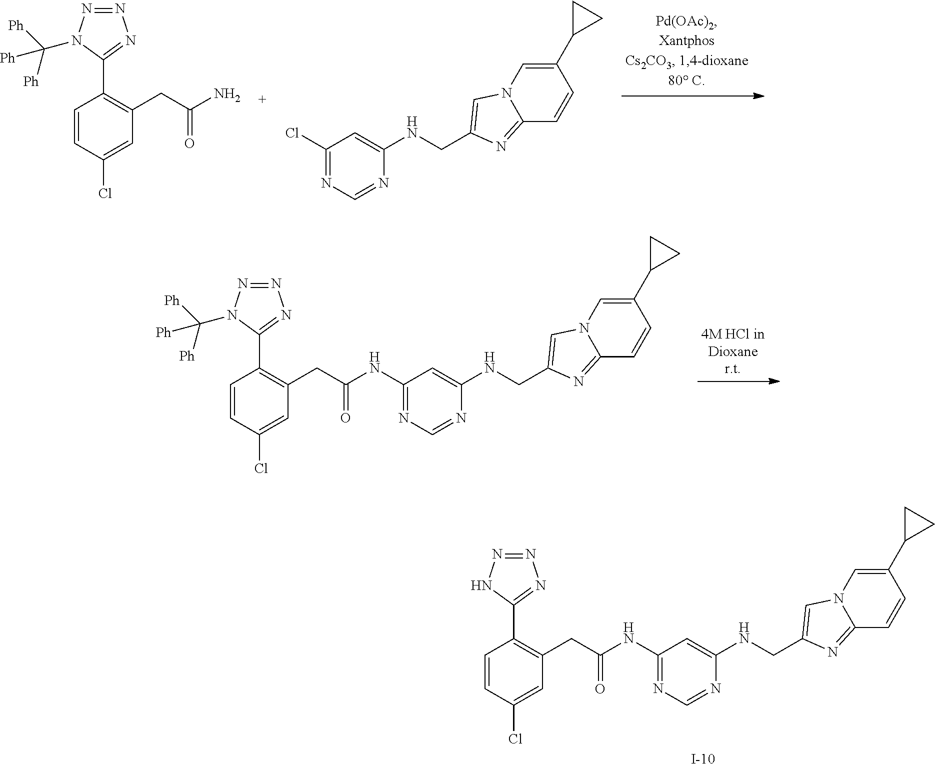

Synthesis of 2-(5-chloro-2-(1-trityl-1H-tetrazol-5-yl)phenyl)-N-(6-(((6-cyclopropylimidazo[1,2-a]pyridin-2-yl)methyl)amino)pyrimidin-4-yl)acetamide. The title compound (0.21 g, 18%) was prepared using a similar procedure to that used for 2-(7-chloroimidazo[1,5-a]pyridin-1-yl)-N-(6-(((6-cyclopropylimidazo[1,2-a]pyridin-2-yl)methyl)amino)pyrimidin-4-yl)acetamide (Example 1) using 6-chloro-N-((6-cyclopropylimidazo[1,2-a]pyridin-2-yl)methyl)pyrimidin-4-amine and 2-(5-chloro-2-(1-trityl-1H-tetrazol-5-yl)phenyl)acetamide. Following purification by silica gel column chromatography using a DCM to DCM/MeOH (9:1) gradient the title compound was used directly in the subsequent step without further purification.

Synthesis of 2-(5-chloro-2-(1H-tetrazol-5-yl)phenyl)-N-(6-(((6-cyclopropylimidazo[1,2-a]pyridin-2-yl)methyl)amino)pyrimidin-4-yl)acetamide. To a stirred solution of 2-(5-chloro-2-(1-trityl-1H-tetrazol-5-yl)phenyl)-N-(6-(((6-cyclopropylimidazo[1,2-a]pyridin-2-yl)methyl)amino)pyrimidin-4-yl)acetamide (0.016 g, 0.022 mmol) in 1,4-dioxane (1 mL) at room temperature was added HCl in dioxane (0.11 mL, 1M, 0.11 mmol) and the resulting mixture was stirred at room temperature for 2 h. Further HCl in dioxane (0.11 mL, 1M, 0.11 mmol) was added and the reaction was stirred at room temperature for 24 h. The solvent was then removed in vacuo. The residue was suspended in DCM (4 mL), MP-carbonate resin (10 equivalents) was added and the mixture stirred for 1 h. The resin was removed by filtration and the filtrate was concentrated in vacuo and the residue was triturated with diisopropylether to give the title compound (4 mg, 37%) as a yellow solid. ESI-MS (M+H)⁺: 501.3, ¹H NMR (400 MHz, DMSO) δ10.72 (s, 1H), 8.23 (s, 1H), 8.06 (s, 1H), 7.74-7.68 (m, 2H), 7.55 (s, 1H), 7.45-7.41 (m, 2H), 7.32 (d, J=9.5 Hz, 1H), 7.19 (s, 1H), 6.98 (s, 1H), 6.88 (d, J=5.9 Hz, 1H), 4.44 (s, 2H), 4.02 (s, 2H), 1.82-1.78 (m, 1H), 0.83-0.77 (m, 2H), 0.56-0.52 (m, 2H).

### Example 11

### Synthesis of 4-chloro-2-(2-((6-(((6-cyclopropylimidazo[1,2-a]pyridin-2-yl)methyl)amino)pyrimidin-4-yl)amino)-2-oxoethyl)benzamide (I-11)

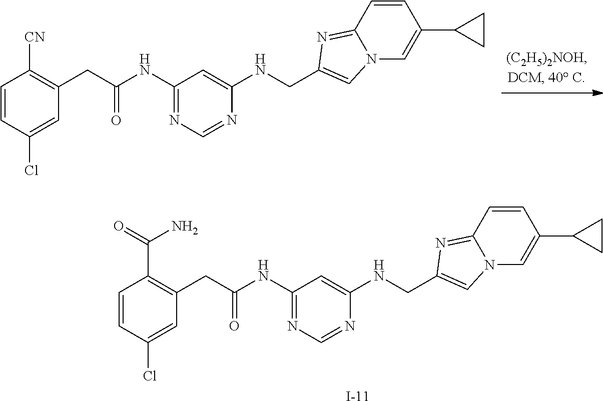

N,N-diethylhydroxylamine (0.054 mL, 0.524 mmol) was added to a solution of 2-(5-chloro-2-cyanophenyl)-N-(6-(((6-cyclopropylimidazo[1,2-a]pyridin-2-yl)methyl)amino)pyrimidin-4-yl)acetamide (40 mg, 0.0874 mmol, Example 2) in DCM (1 mL) and the mixture was heated to 40° C. for 72 h. An additional amount of N,N-diethylhydroxylamine (0.054 mL, 0.524 mmol) was added at 18 h and 42 h. The mixture was then concentrated in vacuo. Purification of the residue by column chromatography on silica gel, eluting with a gradient of 0-10% MeOH in DCM followed by purification by reverse phase C₁₈ preparative HPLC gave the title compound (4.3 mg, 10%). ESI-MS (M+H)⁺: 476.2, ¹H NMR (400 MHz, DMSO) δ10.57 (s, 1H), 8.29 (t, J=0.8 Hz, 1H), 8.19 (d, J=0.9 Hz, 1H), 8.01 (s, 1H), 7.83 (s, 1H), 7.60 (s, 1H), 7.57-7.52 (m, 2H), 7.44-7.40 (m, 2H), 7.37 (d, J=9.4 Hz, 1H), 7.20 (s, 1H), 6.96 (dd, J=1.7, 9.4 Hz, 1H), 4.56 (s, 2H), 3.95 (s, 2H), 1.95-1.87 (m, 1H), 0.94-0.88 (m, 2H), 0.69-0.64 (m, 2H).

### Example 12

### Synthesis of 2-(7-chloroimidazo[1,5-a]pyridin-1-yl)-N-(6-(((6-cyclopropyl-8-(2-oxopyrrolidin-1-yl)imidazo[1,2-a]pyridin-2-yl)methyl)amino)pyrimidin-4-yl)acetamide (I-12)

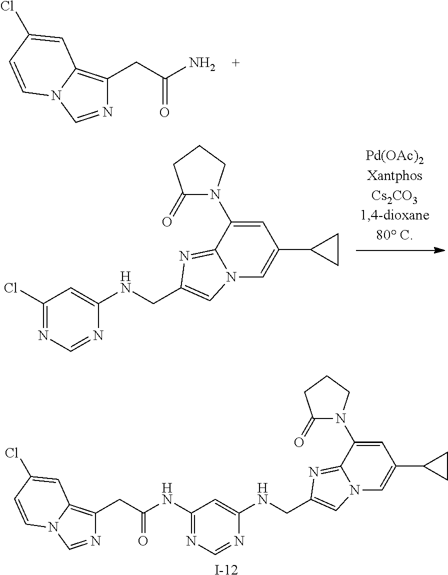

A suspension of 1-(2-(((6-chloropyrimidin-4-yl)amino)methyl)-6-cyclopropylimidazo[1,2-a]pyridin-8-yl)pyrrolidin-2-one (52 mg 0.135 mmol), 2-(7-chloroimidazo[1,5-a]pyridin-1-yl)acetamide (28 mg, 0.135 mmol), Pd(OAc)₂ (6.1 mg, 0.027 mmol), Xantphos (31 mg, 0.054 mmol) and Cs₂CO₃ (66 mg, 0.203 mmol) in 1,4-dioxane (1.5 mL) was degassed with N₂ for 10 minutes then heated to 80° C. for 6 h. The mixture was cooled to room temperature, diluted with water (20 mL) then extracted with EtOAc (3×20 mL). The combined organics were dried over MgSO₄ then concentrated in vacuo. The residue was purified by reverse phase preparative HPLC to give the title compound (3 mg, 4%). ESI-MS (M+H)⁺: 556.3, ¹H NMR (400 MHz, CDCl₃) δ9.42 (s, 1H), 8.30 (s, 1H), 8.14 (s, 1H), 7.84-7.82 (m, 1H), 7.74 (s, 1H), 7.43 (d, J=5.8 Hz, 2H), 7.34 (s, 1H), 7.26-7.22 (m, 1H), 6.57-6.53 (m, 1H), 5.71 (s, 1H), 4.67-4.64 (m, 2H), 4.32-4.27 (m, 2H), 3.93-3.91 (m, 2H), 2.66-2.60 (m, 2H), 2.30-2.21 (m, 2H), 1.92-1.85 (m, 1H), 0.97-0.91 (m, 2H), 0.71-0.66 (m, 2H).

### Example 13

### Synthesis of 2-(7-chloroimidazo[1,5-a]pyridin-1-yl)-N-(6-(((8-cyano-6-cyclopropylimidazo[1,2-a]pyridin-2-yl)methyl)amino)pyrimidin-4-yl)acetamide (I-13)

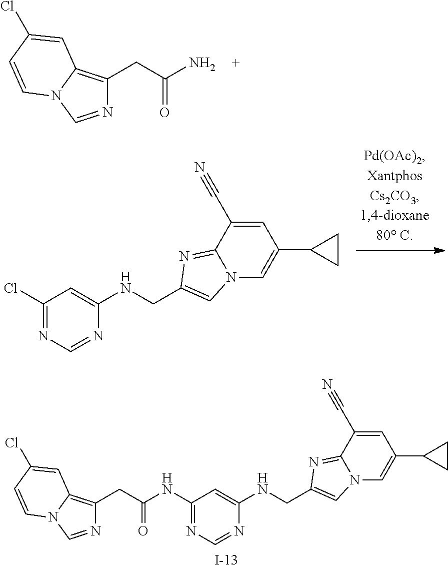

A suspension of 2-(((6-chloropyrimidin-4-yl)amino)methyl)-6-cyclopropylimidazo[1,2-a]pyridine-8-carbonitrile (77 mg 0.239 mmol), 2-(7-chloroimidazo[1,5-a]pyridin-1-yl)acetamide (50 mg, 0.239 mmol), Pd(OAc)₂ (11 mg, 0.048 mmol), Xantphos (55 mg, 0.095 mmol) and Cs₂CO₃ (117 mg, 0.385 mmol) in 1,4-dioxane (4.0 mL) was degassed with N₂ for 10 minutes then heated to 80° C. for 6 h. The mixture was cooled to room temperature, diluted with water (20 mL), then extracted with EtOAc (3×20 mL). The combined organics were dried over MgSO₄ then concentrated in vacuo. The residue was purified by column chromatography on silica gel eluting with 2-10% MeOH in DCM to give the title compound (22 mg, 18%) as an off-white solid. ESI-MS (M+H)⁺: 498.3, ¹H NMR (400 MHz, DMSO) δ10.47 (s, 1H), 8.64 (dd, J=0.3, 1.6 Hz, 1H), 8.35 (s, 1H), 8.33 (dd, J=0.8, 7.4 Hz, 1H), 8.22 (d, J=0.8 Hz, 1H), 7.97 (s, 1H), 7.84 (s, 1H), 7.78 (s, 1H), 7.74 (d, J=1.8 Hz, 1H), 7.27 (s, 1H), 6.67 (dd, J=2.1, 7.6 Hz, 1H), 4.62 (s, 2H), 3.96 (s, 2H), 2.00-1.93 (m, 1H), 0.98-0.93 (m, 2H), 0.77-0.73 (m, 2H).

### Example 14

### Synthesis of 2-(7-chloroimidazo[1,5-a]pyridin-1-yl)-N-(6-(((6-cyclopropyl-8-(3-hydroxyoxetan-3-yl)imidazo[1,2-a]pyridin-2-yl)methyl)amino)pyrimidin-4-yl)acetamide (I-14)

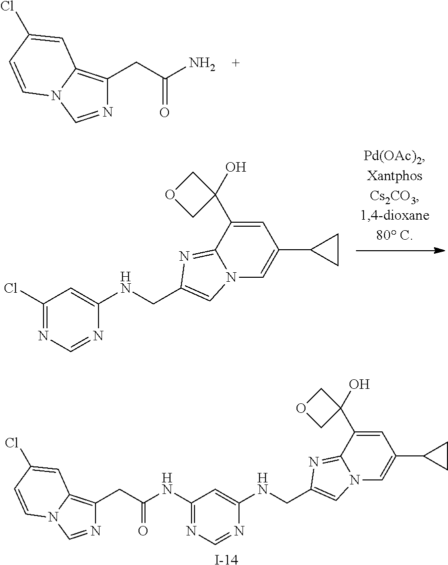

The title compound (22 mg, 26%) was prepared using a similar procedure to that used for 2-(7-chloroimidazo[1,5-a]pyridin-1-yl)-N-(6-(((8-cyano-6-cyclopropylimidazo[1,2-a]pyridin-2-yl)methyl)amino)pyrimidin-4-yl)acetamide (Example 13) by using 3-(2-(((6-chloropyrimidin-4-yl)amino)methyl)-6-cyclopropylimidazo[1,2-a]pyridin-8-yl)oxetan-3-ol and 2-(7-chloroimidazo[1,5-a]pyridin-1-yl)acetamide as coupling partners and purified by preparative HPLC. ESI-MS (M+H)⁺: 545.4, ¹H NMR (400 MHz, DMSO) δ10.49 (s, 1H), 8.40-8.25 (m, 4H), 7.92-7.86 (m, 2H), 7.66 (s, 1H), 7.29 (s, 1H), 7.09 (s, 1H), 6.71 (dd, J=2.0, 7.3 Hz, 1H), 6.51 (s, 1H), 5.27 (d, J=6.6 Hz, 2H), 4.70 (d, J=6.3 Hz, 2H), 4.63 (s, 2H), 4.00 (s, 2H), 2.01-1.93 (m, 1H), 0.99-0.93 (m, 2H), 0.75-0.69 (m, 2H).

### Example 15

### Synthesis of 2-(5-chloro-2-(1H-tetrazol-1-yl)phenyl)-N-(6-(((8-cyano-6-cyclopropylimidazo[1,2-a]pyridin-2-yl)methyl)amino)pyrimidin-4-yl)acetamide I-15)

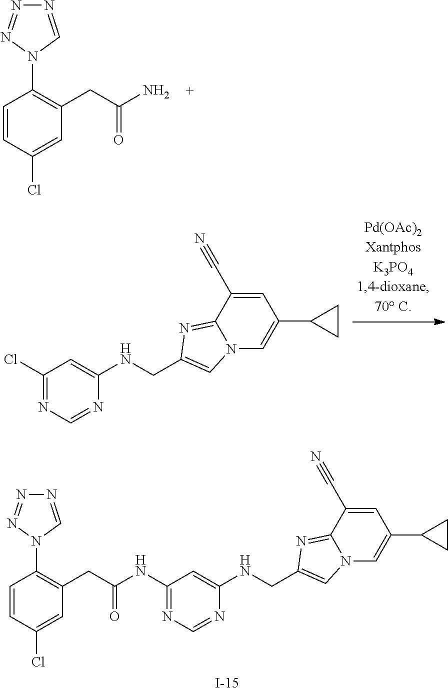

A suspension of 2-(((6-chloropyrimidin-4-yl)amino)methyl)-6-cyclopropylimidazo[1,2-a]pyridine-8-carbonitrile (96 mg 0.30 mmol), 2-(5-chloro-2-(1H-tetrazol-1-yl)phenyl)acetamide (70 mg, 0.30 mmol), Pd(OAc)₂ (13 mg, 0.059 mmol), Xantphos (68 mg, 0.12 mmol) and K₃PO₄ (94 mg, 0.44 mmol) in 1,4-dioxane (5.0 mL) was degassed with N₂ for 10 minutes then heated to 70° C. for 3 h. The mixture was cooled to room temperature, diluted with water (20 mL) then extracted with DCM (3×20 mL). The combined organics were dried over MgSO₄ then concentrated in vacuo. The residue was purified by reverse phase preparative HPLC to give the title compound (12.6 mg, 8%). ESI-MS (M+H)⁺: 526.2, ¹H NMR (400 MHz, DMSO) δ10.44 (s, 1H), 9.79 (s, 1H), 8.65 (d, J=1.1 Hz, 1H), 8.20 (d, J=0.7 Hz, 1H), 7.97 (s, 1H), 7.79-7.73 (m, 3H), 7.68-7.67 (m, 2H), 7.10 (s, 1H), 4.61 (s, 2H), 3.79 (s, 2H), 2.01-1.93 (m, 1H), 0.98-0.93 (m, 2H), 0.78-0.73 (m, 2H).

### Example 16

### Synthesis of 2-(7-chloroimidazo[1,5-a]pyridin-1-yl)-N-(2-(((6-cyclopropylimidazo[1,2-a]pyridin-2-yl)methyl)amino)pyrimidin-4-yl)acetamide (I-16)

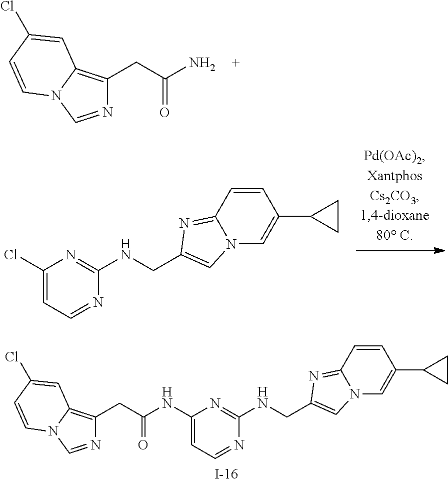

A suspension of 4-chloro-N-((6-cyclopropylimidazo[1,2-a]pyridin-2-yl)methyl)pyrimidin-2-amine (170 mg 0.57 mmol), 2-(7-chloroimidazo[1,5-a]pyridin-1-yl)acetamide (119 mg, 0.57 mmol), Pd(OAc)₂ (51 mg, 0.23 mmol), Xantphos (66 mg, 0.11 mmol) and Cs₂CO₃ (227 mg, 0.85 mmol) in 1,4-dioxane (5.0 mL) was degassed with N₂ for 10 minutes then heated to 80° C. for 1.5 h. The mixture was cooled to room temperature, diluted with water (20 mL) then extracted with DCM (3×20 mL). The combined organics were dried over MgSO₄ then concentrated in vacuo. The residue was purified by reverse phase preparative HPLC to give the title compound (5.7 mg, 2%). ESI-MS (M+H)⁺: 473.3, ¹H NMR (400 MHz, DMSO) δ10.47 (s, 1H), 8.36-8.30 (m, 3H), 8.17 (d, J=5.7 Hz, 1H), 7.82 (s, 1H), 7.60 (s, 1H), 7.40-7.32 (m, 2H), 7.22 (d, J=5.5 Hz, 1H), 6.96 (dd, J=1.7, 9.4 Hz, 1H), 6.67 (dd, J=2.1, 7.5 Hz, 1H), 4.60 (d, J=6.1 Hz, 2H), 3.99 (s, 2H), 1.92 (ddd, J=5.1, 8.4, 13.5 Hz, 1H), 0.92 (ddd, J=4.3, 6.3, 8.4 Hz, 2H), 0.67 (ddd, J=3.3, 4.7, 6.7 Hz, 2H).

### Example 17

Synthesis of 2-(7-chloroimidazo[1,5-a]pyridin-1-yl)-N-(6-(((6-cyclopropyl-8-(4-methylpiperazin-1-yl)imidazo[1,2-a]pyridin-2-yl)methyl)amino)pyrimidin-4-yl)acetamide (I-17)

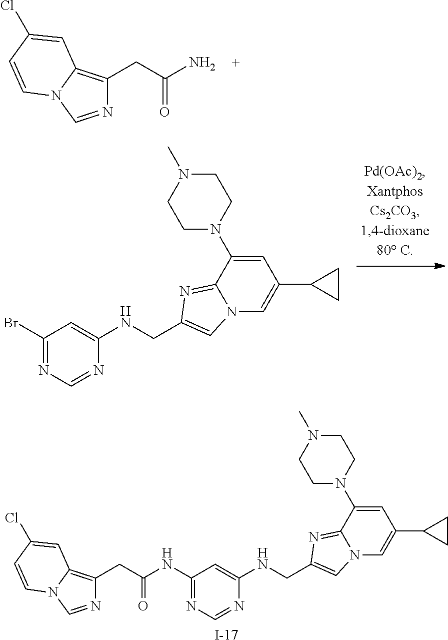

A suspension of 6-bromo-N-((6-cyclopropyl-8-(4-methylpiperazin-1-yl)imidazo[1,2-a]pyridin-2-yl)methyl)pyrimidin-4-amine (95 mg 0.13 mmol), 2-(7-chloroimidazo[1,5-a]pyridin-1-yl)acetamide (30 mg, 0.14 mmol), Pd(OAc)₂ (2.9 mg, 0.013 mmol), Xantphos (15 mg, 0.026 mmol) and Cs₂CO₃ (84 mg, 0.26 mmol) in 1,4-dioxane (5.0 mL) was degassed with N₂ for 10 minutes then heated to 80° C. for 1.5 h. The mixture was cooled to room temperature then diluted with a mixture of methanol in DCM (1:9) and filtered through Celite®. The mixture was washed with water then concentrated in vacuo. The residue was purified by reverse phase preparative HPLC to give the title compound (4.0 mg, 2%). ESI-MS (M+H)⁺: 571.4, ¹H NMR (400 MHz, DMSO) δ10.45 (s, 1H), 8.36-8.32 (m, 2H), 8.22 (s, 1H), 7.85 (d, J=6.4 Hz, 3H), 7.50 (s, 1H), 7.25 (s, 1H), 6.67 (dd, J=2.0, 7.5 Hz, 1H), 6.14 (s, 1H), 4.54-4.54 (m, 2H), 3.95 (s, 2H), 3.48-3.43 (m, 8H), 2.24 (s, 3H), 1.88-1.81 (m, 1H), 0.89-0.83 (m, 2H), 0.68-0.63 (m, 2H).

### Example 18

Synthesis of 2-(7-chloroimidazo[1,5-a]pyridin-1-yl)-N-(6-(((6-cyclopropyl-8-(2-oxopyrrolidin-1-yl imidazo[1,2-a]pyridin-2-yl methyl (methyl amino)pyrimidin-4-yl)acetamide (I-18)

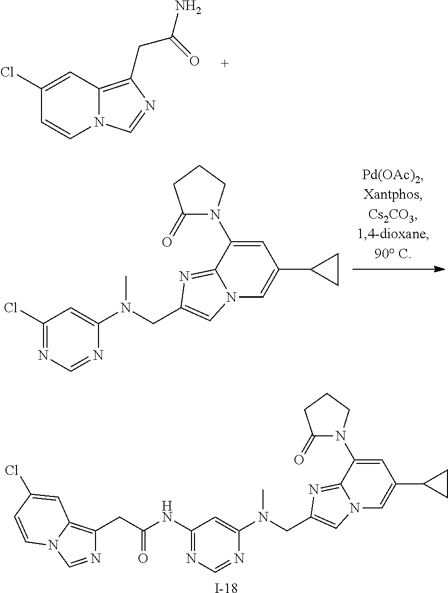

A suspension of 1-(2-(((6-chloropyrimidin-4-yl)(methyl)amino)methyl)-6-cyclopropylimidazo[1,2-a]pyridin-8-yl)pyrrolidin-2-one (63 mg, 0.16 mmol), 2-(7-chloroimidazo[1,5-a]pyridin-1-yl)acetamide (44 mg, 0.21 mmol), Cs₂CO₃ (100 mg, 0.32 mmol), Xantphos (49 mg, 0.084 mmol) and Pd(OAc)₂ (9.4 mg, 0.042 mmol) in anhydrous 1,4-dioxane (2.0 mL) was degassed for 10 minutes and the reaction mixture was then stirred at 90° C. for 4 hours. The reaction mixture was allowed to cool to room temperature and concentrated in vacuo. The residue was suspended in water (5 mL), filtered and washed with water and the collected solid was dried in vacuo. The residue was purified by column chromatography on silica gel, eluting with a gradient of 3-10% MeOH in DCM. The residue was further purified by preparative HPLC to give the title compound (31 mg, 34%) as a pale brown solid. ESI-MS (M+H)⁺: 570.5, ¹H NMR (400 MHz, DMSO) δ10.51 (s, 1H), 8.28-8.21 (m, 3H), 8.10 (s, 1H), 7.75 (s, 1H), 7.52 (s, 1H), 7.33 (s, 1H), 7.07 (s, 1H), 6.59 (dd, J=1.8, 7.3 Hz, 1H), 4.73 (s, 2H), 4.05 (dd, J=6.9, 6.9 Hz, 2H), 3.90 (s, 2H), 3.03 (s, 3H), 2.37 (dd, J=8.1, 8.1 Hz, 2H), 1.99 (dd, J=7.1, 7.1 Hz, 2H), 1.88-1.80 (m, 1H), 0.88-0.81 (m, 2H), 0.59-0.52 (m, 2H).

### Example 19

### Synthesis of: 2-(7-chloroimidazo[1,5-a]pyridin-1-yl)-N-(6-(((6-cyclopropyl-8-(3-fluorooxetan-3-yl)imidazo[1,2-a]pyridin-2-yl)methyl)amino)pyrimidin-4-yl)acetamide (I-19)

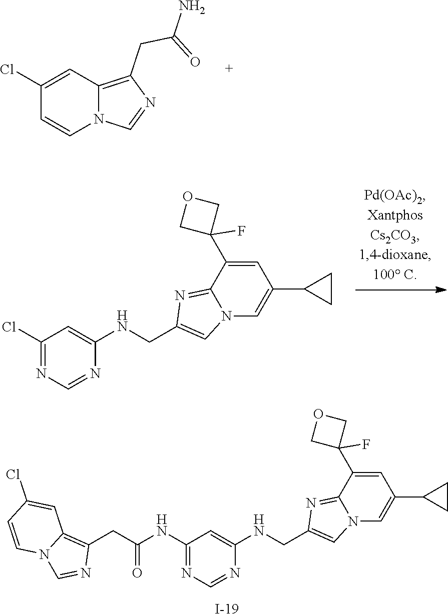

A suspension of 6-chloro-N-((6-cyclopropyl-8-(3-fluorooxetan-3-yl)imidazo[1,2-a]pyridin-2-yl)methyl)pyrimidin-4-amine (70 mg, 0.19 mmol), 2-(7-chloroimidazo[1,5-a]pyridin-1-yl)acetamide (52 mg, 0.25 mmol), Cs₂CO₃ (120 mg, 0.38 mmol), Xantphos (58 mg, 0.10 mmol) and Pd(OAc)₂ (11 mg, 0.050 mmol) in anhydrous 1,4-dioxane (3.0 mL) was degassed for 10 minutes and the reaction mixture was then stirred at 100° C. for 3 hours. The reaction mixture was allowed to cool to room temperature and concentrated in vacuo. The residue was purified by column chromatography on silica gel, eluting with a gradient of 3-10% MeOH in DCM. The residue was further purified by preparative HPLC to give the title compound (22 mg, 21%) as a cream solid. ESI-MS (M+H)⁺: 547.4, ¹H NMR (400 MHz, DMSO) δ10.46 (s, 1H), 8.38-8.31 (m, 3H), 8.22 (s, 1H), 7.90 (s, 1H), 7.84 (s, 1H), 7.66 (s, 1H), 7.26 (s, 1H), 7.12 (t, J=2.1 Hz, 1H), 6.67 (dd, J=2.1, 7.5 Hz, 1H), 5.39-5.29 (m, 2H), 4.97 (dd, J=8.5, 22.4 Hz, 2H), 4.59 (s, 2H), 3.96 (s, 2H), 1.99-1.91 (m, 1H), 0.96-0.90 (m, 2H), 0.74-0.69 (m, 2H).

### Example 20

Synthesis of: ethyl 4-(2-(7-chloroimidazo[1,5-a]pyridin-1-yl)acetamido)-6-(((6-cyclopropylimidazo[1,2-a]pyridin-2-yl)methyl)amino)pyrimidine-2-carboxylate (I-20)

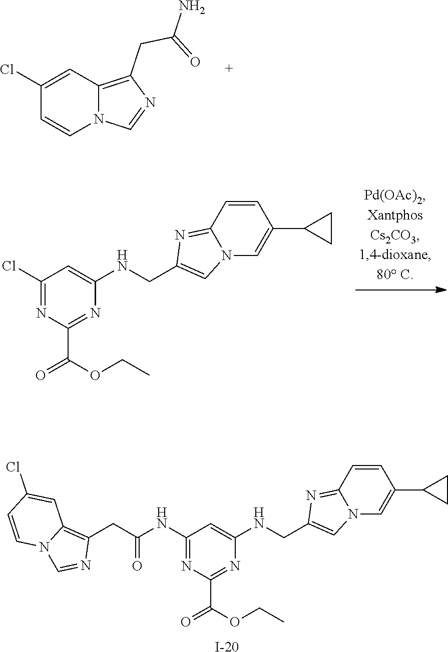

A mixture of ethyl 4-chloro-6-(((6-cyclopropylimidazo[1,2-a]pyridin-2-yl)methyl)amino)pyrimidine-2-carboxylate (130 mg, 0.34 mmol), 2-(7-chloroimidazo[1,5-a]pyridin-1-yl)acetamide (70 mg, 0.34 mmol), Cs₂CO₃ (160 mg, 0.50 mmol), Xantphos (78 mg, 0.13 mmol) and Pd(OAc)₂ (15 mg, 0.067 mmol) was placed under a N₂ atmosphere, anhydrous 1,4-dioxane (8.0 mL) was added and the suspension was degassed for 10 min. The reaction mixture was then stirred at 80° C. for 1.5 hours. The reaction mixture was allowed to cool to room temperature, diluted with 5% EtOH in DCM (50 mL) and washed with water (30 mL). The organics were dried over a hydrophobic frit and concentrated in vacuo. The residue was purified by column chromatography on silica gel, eluting with a gradient of 0-10% EtOH in DCM. The obtained residue was triturated with Et₂O and dried in a vacuum oven to give the title compound (76 mg, 42%) as a yellow solid. ESI-MS (M+H)⁺: 545.4, ¹H NMR (400 MHz, DMSO) δ10.79 (s, 1H), 8.35-8.28 (m, 3H), 8.20-8.17 (m, 1H), 7.82 (s, 1H), 7.65 (s, 1H), 7.37 (d, J=9.2 Hz, 2H), 6.97 (dd, J=1.8, 9.3 Hz, 1H), 6.66 (dd, J=2.1, 7.5 Hz, 1H), 4.63 (s, 2H), 4.31 (q, J=7.1 Hz, 2H), 3.97 (s, 2H), 1.95-1.87 (m, 1H), 1.31 (t, J=7.1 Hz, 3H), 0.95-0.89 (m, 2H), 0.69-0.64 (m, 2H)

### Example 21

### Synthesis of 4-(2-(7-chloroimidazo[1,5-a]pyridin-1-yl)acetamido)-6-(((6-cyclopropylimidazo[1,2-a]pyridin-2-yl)methyl)amino)pyrimidine-2-carboxylic acid (I-21)

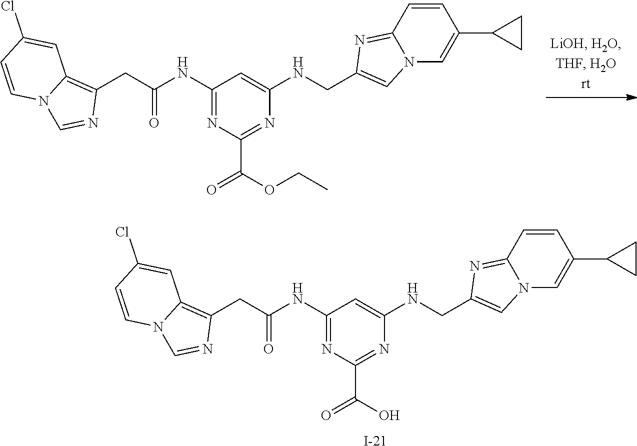

Lithium hydroxide (4.4 mg, 0.105 mmol) was added to a solution of ethyl 4-(2-(7-chloroimidazo[1,5-a]pyridin-1-yl)acetamido)-6-(((6-cyclopropylimidazo[1,2-a]pyridin-2-yl)methyl)amino)pyrimidine-2-carboxylate (52 mg, 0.095 mmol, Example 20) in a mixture of THF (4 mL), water (0.8 mL) and stirred at room temperature for 1 h. The mixture was concentrated in vacuo, and the residue was suspended in 1,4-dioxane (10 mL). HCl in 1,4-dioxane (4 M, 30 μL) was added and stirred for 5 min. The mixture was concentrated in vacuo, the solid was triturated with water (15 mL) and DMSO (3 ml) to give the title compound. ESI-MS (M+H)⁺: 517.3, ¹H NMR (400 MHz, DMSO) 12.15-12.15 (m, 1H), 8.34-8.26 (m, 3H), 7.93-7.74 (m, 3H), 7.38 (d, J=9.4 Hz, 1H), 7.21 (s, 1H), 6.96 (d, J=9.3 Hz, 1H), 6.65-6.62 (m, 1H), 4.62-4.62 (m, 2H), 4.11-4.11 (m, 2H), 1.94-1.87 (m, 1H), 0.93-0.87 (m, 2H), 0.69-0.64 (m, 2H).

### Example 22

### Synthesis of 2-(7-chloroimidazo[1,5-a]pyridin-1-yl)-N-(6-((8-cyano-6-cyclopropylimidazo[1,2-a]pyridin-2-yl)methoxy)pyrimidin-4-yl)acetamide (I-22)

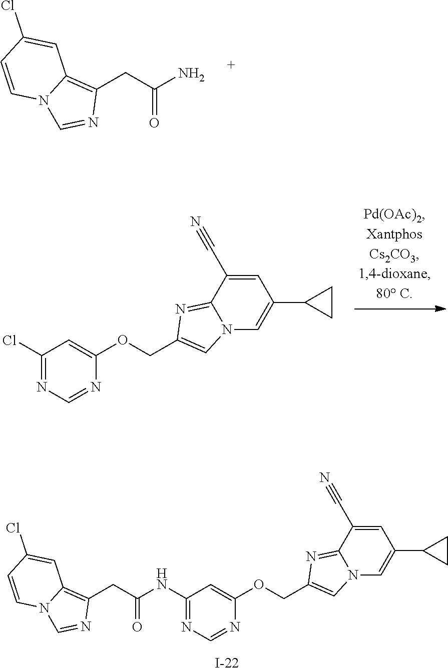

A mixture of 2-(((6-chloropyrimidin-4-yl)oxy)methyl)-6-cyclopropylimidazo[1,2-a]pyridine-8-carbonitrile (58 mg, 0.18 mmol), 2-(7-chloroimidazo[1,5-a]pyridin-1-yl)acetamide (37 mg, 0.18 mmol), Cs₂CO₃ (87 mg, 0.27 mmol), Xantphos (41 mg, 0.071 mmol) and Pd(OAc)₂ (8.0 mg, 0.036 mmol) was placed under a N₂ atmosphere, anhydrous 1,4-dioxane (3.5 mL) was added and the suspension was degassed for 10 minutes. The reaction mixture was then stirred at 80° C. for 2 hours. The reaction mixture was allowed to cool to room temperature, diluted with 5% MeOH in DCM (10 mL), filtered through Celite® and concentrated in vacuo. The residue was triturated with MeOH (×2). The residue was heated in DMSO (5 mL), filtered, washed with water (5 mL) and dried to give the title compound (4 mg, 4%). ESI-MS (M+H)⁺: 499.3, ¹H NMR (400 MHz, DMSO) δ11.07 (s, 1H), 8.72 (s, 1H), 8.64 (s, 1H), 8.41-8.35 (m, 2H), 8.07 (s, 1H), 7.85 (d, J=17.4 Hz, 2H), 7.50 (s, 1H), 6.72 (d, J=7.3 Hz, 1H), 5.58 (s, 2H), 4.06 (s, 2H), 2.02 (s, 1H), 1.03-1.00 (m, 2H), 0.82-0.79 (m, 2H).

### Example 23

### Synthesis of 2-(7-chloroimidazo[1,5-a]pyridin-1-yl)-N-(4-((6-cyclopropylimidazo[1,2-a]pyridin-2-yl)methoxy)pyridin-2-yl)acetamide (I-23)

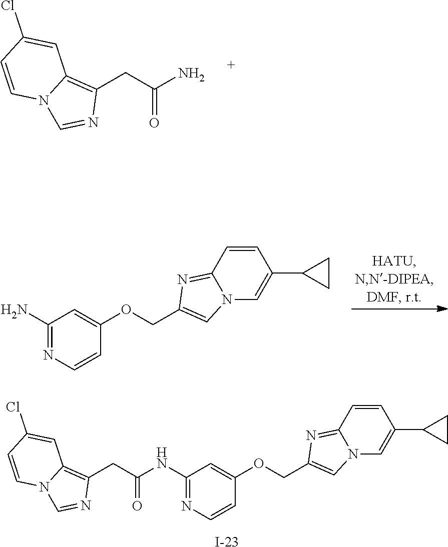

A solution of 4-((6-cyclopropylimidazo[1,2-a]pyridin-2-yl)methoxy)pyridin-2-amine (64 mg, 0.23 mmol), 2-(7-chloroimidazo[1,5-a]pyridin-1-yl)acetic acid (53 mg, 0.25 mmol), HATU (95 mg, 0.25 mmol) and DIPEA (0.080 mL, 0.46 mmol) in anhydrous DMF (3.0 mL) was stirred at room temperature for 16 hours. The reaction mixture was diluted with water (50 mL) and brine (sat. aq., 50 mL) and extracted with EtOAc (3×50 mL). The combined organics were dried over MgSO₄ and concentrated in vacuo. The residue was purified by preparative HPLC to give the title compound (36 mg, 33%). ESI-MS (M+H)⁺: 473.2, ¹H NMR (400 MHz, DMSO) δ10.60 (s, 1H), 8.36 (d, J=0.6 Hz, 1H), 8.34 (ddd, J=0.8, 0.8, 1.6 Hz, 1H), 8.33 (dd, J=0.9, 7.5 Hz, 1H), 8.15 (d, J=5.8 Hz, 1H), 7.87 (s, 1H), 7.85 (ddd, J=0.9, 0.9, 2.0 Hz, 1H), 7.77 (d, J=2.3 Hz, 1H), 7.43 (d, J=9.4 Hz, 1H), 7.02 (dd, J=1.8, 9.3 Hz, 1H), 6.84 (dd, J=2.4, 5.8 Hz, 1H), 6.67 (dd, J=2.1, 7.5 Hz, 1H), 5.23 (s, 2H), 3.98 (s, 2H), 1.94 (tdd, J=3.9, 9.3, 9.3 Hz, 1H), 0.93 (ddd, J=4.3, 6.3, 8.4 Hz, 2H), 0.69 (ddd, J=3.2, 4.6, 6.7 Hz, 2H).

### Example 24

### Synthesis of N-(4-(((6-cyclopropyl-8-(2-oxopyrrolidin-1-yl)imidazo[1,2-a]pyridin-2-yl)methyl)amino)pyridin-2-yl)-2-(3-fluoro-4-methoxypyridin-2-yl)acetamide formic acid salt (I-24)

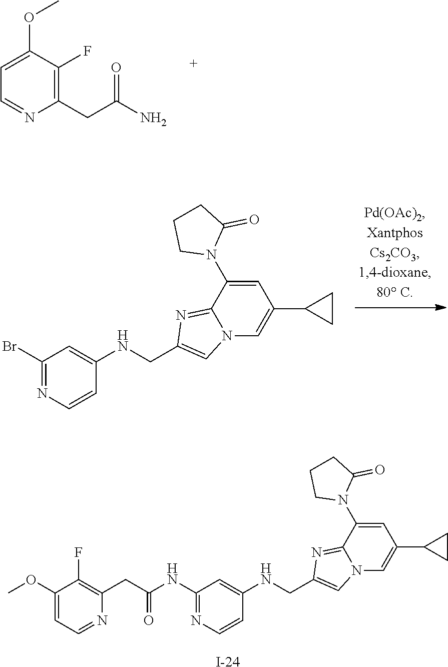

A suspension of 1-(2-(((2-bromopyridin-4-yl)amino)methyl)-6-cyclopropylimidazo[1,2-a]pyridin-8-yl)pyrrolidin-2-one (77 mg, 0.18 mmol), 2-(3-fluoro-4-methoxypyridin-2-yl)acetamide (33 mg, 0.18 mmol), Xantphos (42 mg, 0.072 mmol) and Cs₂CO₃ (120 mg, 0.36 mmol) in anhydrous 1,4-dioxane (2.0 mL) was degassed for 10 minutes. Pd(OAc)₂ (8.1 mg, 0.036 mmol) was added and the reaction mixture was stirred at 80° C. for 5 hours. The reaction mixture was allowed to cool to room temperature, diluted with NaHCO₃ (sat. aq., 15 mL) and brine (sat. aq., 15 mL) and extracted with EtOAc (3×25 mL). The combined organics were dried over MgSO₄ and concentrated in vacuo. The residue was dissolved in 50% MeOH in DCM and loaded onto an SCX cartridge, washed with 50% MeOH in DCM, eluted with 50% 7 N NH₃ in MeOH in DCM and the eluent was concentrated in vacuo. The residue was purified by preparative HPLC to give the title compound (21 mg, 22%) as a yellow solid. ESI-MS (M+H)⁺: 530.4, ¹H NMR (400 MHz, DMSO) δ10.25 (s, 1H), 8.23-8.18 (m, 3H), 7.77 (d, J=5.8 Hz, 1H), 7.65 (s, 1H), 7.38 (br s, 1H), 7.17 (dd, J=5.7, 6.5 Hz, 1H), 7.14-7.09 (m, 2H), 6.32 (dd, J=2.0, 5.8 Hz, 1H), 4.34 (d, J=5.8 Hz, 2H), 4.13 (app t, J=7.1 Hz, 2H), 3.92 (s, 3H), 3.89 (d, J=2.5 Hz, 2H), 2.48-2.43 (m, 2H), 2.12-2.07 (m, 2H), 1.94-1.86 (m, 1H), 0.94-0.87 (m, 2H), 0.66-0.60 (m, 2H).

### Example 25

### Synthesis of 2-(7-chloroimidazo[1,5-a]pyridin-1-yl)-N-(4-(((6-cyclopropylimidazo[1,2-a]pyridin-2-yl)methyl)amino)pyridin-2-yl)acetamide formic acid salt (I-25)

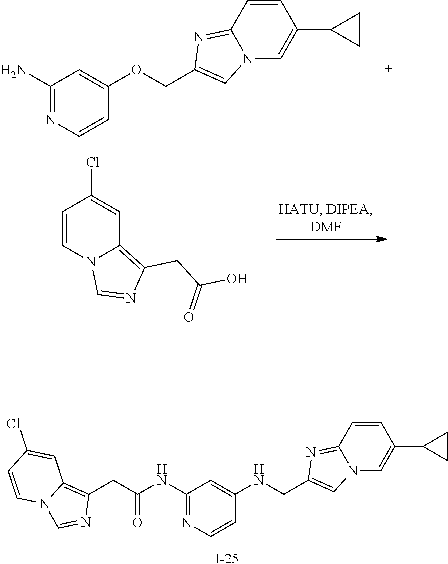

HATU (0.1 g, 0.276 mmol) was added to a mixture of N₄-((6-cyclopropylimidazo[1,2-a]pyridin-2-yl)methyl)pyridine-2,4-diamine (0.07 g, 0.251 mmol), 2-(7-chloroimidazo[1,5-a]pyridin-1-yl)acetic acid (0.058 g, 0.276 mmol) and DIPEA (0.087 mL, 0.501 mmol) in DMF (3.5 mL) at room temperature. The mixture was diluted with water (50 mL), brine (50 mL) and extracted with EtOAc (3×50 mL), the organic phases were combined, dried (MgSO₄), filtered and concentrated in vacuo. Purification by reverse phase preparative HPLC gave the titled compound (25 mg, 24%). ESI-MS (M+H)⁺: 472.2, ¹H NMR (400 MHz, DMSO) δ11.22 (br s, 1H), 8.36-8.32 (m, 4H), 8.13 (s, 0.5H), 7.83-7.79 (m, 2H), 7.73 (s, 1H), 7.42 (d, J=9.3 Hz, 1H), 7.06-7.01 (m, 1H), 6.70-6.63 (m, 3H), 4.50 (d, J=5.4 Hz, 2H), 3.99 (s, 2H), 1.97-1.89 (m, 1H), 0.96-0.89 (m, 2H), 0.70-0.64 (m, 2H).

The examples in the table below were synthesised in a similar the same manner as described above for 2-(7-chloroimidazo[1,5-a]pyridin-1l-yl)-N-(4-(((6-cyclopropylimidazo[1,2-a]pyridin-2-yl)methyl)amino)pyridin-2-yl)acetamide (Example 25) using N⁴-((6-cyclopropylimidazo[1,2-a]pyridin-2-yl)meth 1)pyridine-2,4-diamine and an appropriate coupling partner.

| | | Coupling | |
|---|---|---|---|
| EG | Compound | Partner | Analytical Data |
| 26 | 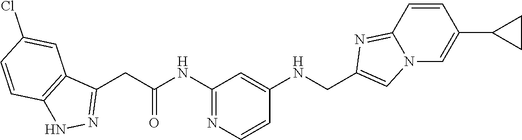 [SMILES: O=C(Cc1n[nH]c2ccc(Cl)cc12)Nc1cc(NCc2cn3cc(C4CC4)ccc3n2)ccn1] 2-(5-chloro-1H-indazol-3-yl)-N-(4-(((6-cyclopropylimidazo[1,2-a]pyridin-2- yl)methyl)amino)pyridin-2-yl)acetamide (I-26) | 2-(5- chloro- 1H- indazol- 3-yl)acetic acid | ESI-MS (M + H)⁺: 472.2, ¹H NMR (400 MHz, DMSO) δ 13.04 (s, 1H), 10.33 (s, 1H), 8.29 (s, 1H), 7.92 (d, J = 1.7 Hz, 1H), 7.80 (d, J = 5.8 Hz, 1H), 7.62 (s, 1H), 7.53 (d, J = 8.8 Hz, 1H), 7.42-7.33 (m, 3H), 7.12 (s, 1H), 6.97 (dd, J = 1.7, 9.4 Hz, 1H), 6.34 (dd, J = 2.1, 5.8 Hz, 1H), 4.34 (d, J = 5.8 Hz, 2H), 4.05 (s, 2H), 1.91 (s, 1H), 0.93-0.89 (m, 2H), 0.69-0.65 (m, 2H). |
| 27 | 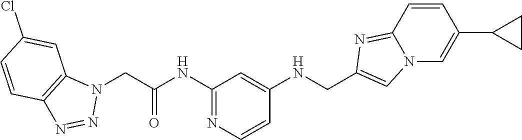 [SMILES: O=C(Cn1nnc2ccc(Cl)cc21)Nc1cc(NCc2cn3cc(C4CC4)ccc3n2)ccn1] 2-(6-chloro-1H-benzo[d][1,2,3]triazol-1-yl)-N-(4-(((6-cyclopropylimidazo[1,2- a]pyridin-2-yl)methyl)amino)pyridin-2-yl)acetamide (I-27) | 2-(6- chloro- 1H-benzo [d][1,2, 3]triazol-1- yl)acetic acid | Purified by Chiral SFC ESI-MS (M + H)⁺: 473, ¹H NMR (400 MHz, DMSO) δ 10.73 (s, 1H), 8.33 (ddd, J = 0.8, 0.8, 1.6 Hz, 1H), 8.18- 8.14 (m, 2H), 7.88 (d, J = 5.8 Hz, 1H), 7.65 (s, 1H), 7.51 (dd, J = 1.9, 8.8 Hz, 1H), 7.41 (d, J = 9.3 Hz, 1H), 7.35 (br s, 1H), 7.18 (t, J = 5.6 Hz, 1H), 7.01 (dd, J = 1.8, 9.3 Hz, 1H), 6.44 (dd, J = 2.1, 5.8 Hz, 1H), 5.77 (s, 2H), 4.37 (d, J = 5.7 Hz, 2H), 1.99-1.91 (m, 1H), 0.99- 0.93 (m, 2H), 0.73-0.68 (m, 2H). |
| 28 | 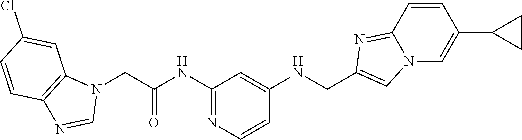 [SMILES: O=C(Cn1cnc2ccc(Cl)cc21)Nc1cc(NCc2cn3cc(C4CC4)ccc3n2)ccn1] 2-(6-chloro-1H-benzo[d]imidazol-1-yl)-N-(4-(((6-cyclopropylimidazo[1,2- a]pyridin-2-yl)methyl)amino)pyridin-2-yl)acetamide (I-28) | 2-(6- chloro- 1H- benzo[d] imidazol- 1-yl)acetic acid | Purified by Chiral SFC ESI-MS (M + H)⁺: 472.8, ¹H NMR (400 MHz, DMSO) δ 10.53 (s, 1H), 8.29 (s, 2H), 7.82 (d, J = 5.9 Hz, 1H), 7.73 (d, J = 2.0 Hz, 1H), 7.68 (d, J = 8.6 Hz, 1H), 7.61 (s, 1H), 7.37 (d, J = 9.3 Hz, 1H), 7.33 (br s, 1H), 7.24 (dd, J = 2.1, 8.6 Hz, 1H), 7.13 (t, J = 5.6 Hz, 1H), 6.96 (dd, J = 1.8, 9.3 Hz, 1H), 6.38 (dd, J = 2.1, 5.8 Hz, 1H), 5.20 (s, 2H), 4.33 (d, J = 5.7 Hz, 2H), 1.95-1.87 (m, 1H), 0.94- 0.88 (m, 2H), 0.68-0.64 (m, 2H). |
| 29 | 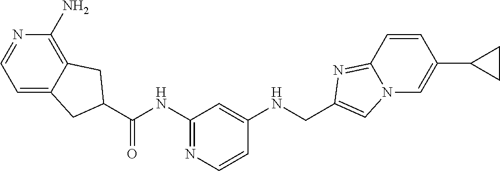 [SMILES: Nc1nccc2c1CC(C(=O)Nc1cc(NCc3cn4cc(C5CC5)ccc4n3)ccn1)C2] 1-amino-N-(4-(((6-cyclopropylimidazo[1,2-a]pyridin-2- yl)methyl)amino)pyridin-2-yl)-6,7-dihydro-5H-cyclopenta[c]pyridine-6- carboxamide (I-29) | 1-amino- 6,7- dihydro- 5H- cyclopenta [c] pyridine-6- carboxylic acid hydro- chloride | ESI-MS (M + H)⁺: 440.3, ¹H NMR (400 MHz, DMSO) δ 10.37 (s, 1H), 8.53 (s, 1H), 8.00 (d, J = 5.6 Hz, 1H), 7.94 (d, J = 4.1 Hz, 1H), 7.85 (s, 1H), 7.67 (s, 1H), 7.59 (d, J = 10.9 Hz, 1H), 7.34- 7.30 (m, 1H), 7.19 (d, J = 10.2 Hz, 1H), 6.67 (d, J = 5.6 Hz, 1H), 6.56 (d, J = 5.3 Hz, 1H), 5.90 (s, 2H), 4.57 (d, J = 5.3 Hz, 2H), 3.72-3.66 (m, 1H), 3.26- 2.99 (m, 4H), 2.17-2.09 (m, 1H), 1.16- 1.10 (m, 2H), 0.90-0.85 (m, 2H). |

### Example 30

### Synthesis of 2-(7-chloroimidazo[1,5-a]pyridin-1-yl)-N-(4-(((8-cyano-6-cyclopropylimidazo[1,2-a]pyridin-2-yl)methyl)amino)pyridin-2-yl)acetamide (I-30)

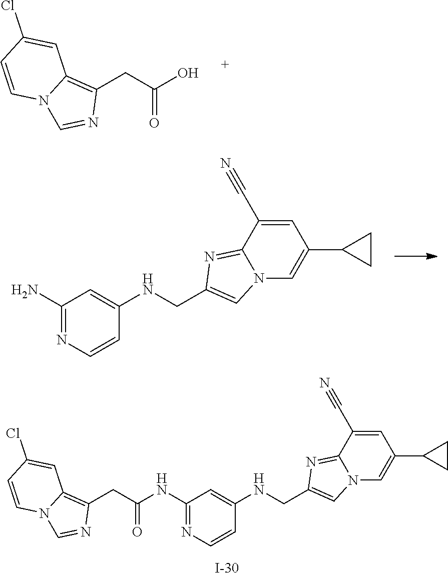

The title compound (5.7 mg, 13%) was prepared using a similar procedure to that used for 2-(7-chloroimidazo[1,5-a]pyridin-1-yl)-N-(4-(((6-cyclopropylimidazo[1,2-a]pyridin-2-yl)methyl)amino)pyridin-2-yl)acetamide (Example 25) by using 2-(((2-aminopyridin-4-yl)amino)methyl)-6-cyclopropylimidazo[1,2-a]pyridine-8-carbonitrile and 2-(7-chloroimidazo[1,5-a]pyridin-1-yl)acetic acid as coupling partners and purified by preparative HPLC. ESI-MS (M+H)⁺: 497.3, ¹H NMR (400 MHz, DMSO) δ10.16 (s, 1H), 8.69 (d, J=1.5 Hz, 1H), 8.40-8.34 (m, 2H), 7.89-7.87 (m, 1H), 7.84-7.81 (m, 2H), 7.79 (d, J=1.5 Hz, 1H), 7.46-7.45 (m, 1H), 7.27 (t, J=5.9 Hz, 1H), 6.70 (dd, J=2.3, 7.4 Hz, 1H), 6.37 (dd, J=2.1, 5.7 Hz, 1H), 4.45 (d, J=5.8 Hz, 2H), 3.96 (s, 2H), 2.04-1.96 (m, 1H), 1.05-0.96 (m, 2H), 0.82-0.76 (m, 2H).

### Example 31

### Synthesis of 2-(7-chloroimidazo[1,5-a]pyridin-1-yl)-N-(5-(((6-cyclopropylimidazo[1,2-a]pyridin-2-yl)methyl)amino)pyridazin-3-yl)acetamide (I-31)

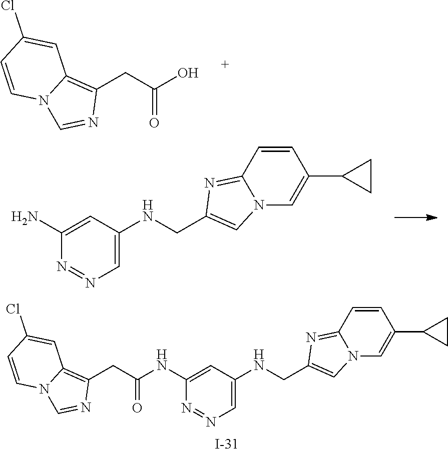

The title compound (15 mg, 13%) was prepared using a similar procedure to that used for 2-(7-chloroimidazo[1,5-a]pyridin-1-yl)-N-(4-(((6-cyclopropylimidazo[1,2-a]pyridin-2-yl)methyl)amino)pyridin-2-yl)acetamide (Example 25) by using N⁵-((6-cyclopropylimidazo[1,2-a]pyridin-2-yl)methyl)pyridazine-3,5-diamine and 2-(7-chloroimidazo[1,5-a]pyridin-1-yl)acetic acid as coupling partners. ESI-MS (M+H)⁺: 473.2, ¹H NMR (400 MHz, DMSO) δ10.71-10.67 (m, 1H), 8.46-8.42 (m, 1H), 8.36-8.28 (m, 3H), 7.85-7.82 (m, 1H), 7.68-7.66 (m, 1H), 7.57-7.51 (m, 1H), 7.46 (s, 1H), 7.39 (d, J=9.3 Hz, 1H), 7.01-6.95 (m, 1H), 6.69-6.64 (m, 1H), 4.41-4.35 (m, 2H), 3.99-3.95 (m, 2H), 1.96-1.87 (m, 1H), 0.96-0.88 (m, 2H), 0.70-0.64 (m, 2H).

### Example 32

### Synthesis of 2-(7-chloroimidazo[1,5-a]pyridin-1-yl)-N-(5-((6-cyclopropylimidazo[1,2-a]pyridin-2-yl)methoxy)pyridazin-3-yl)acetamide (I-32)

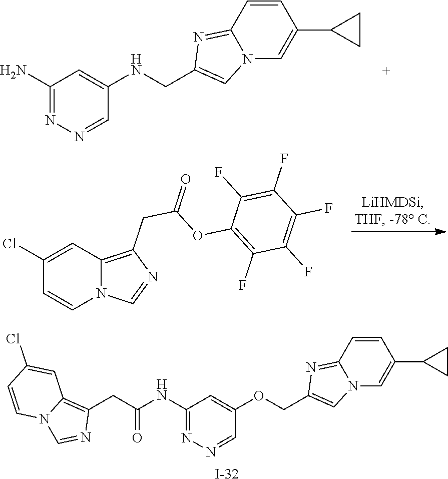

Lithium bis(trimethylsilyl)amide (1M, THF, 0.19 mL) was added to a cooled stirred solution of 5-((6-cyclopropylimidazo[1,2-a]pyridin-2-yl)methoxy)pyridazin-3-amine (49 mg, 0.174 mmol) in THF (2 mL) at −78° C. After 45 min, a solution of perfluorophenyl 2-(7-chloroimidazo[1,5-a]pyridin-1-yl)acetate (66 mg, 0.174 mmol) in THF (2 mL) was added dropwise and the mixture was stirred at −78° C. for 2 h followed by room temperature for 18 h. The mixture was diluted with EtOAc (70 mL) and washed with NaHCO₃ (10 mL), water (10 mL) and brine (10 mL), dried (MgSO₄) and concentrated in vacuo. Purification by reverse phase preparative HPLC gave the titled compound (13 mg, 16%). ESI-MS (M+H)⁺: 474.3, ¹H NMR (400 MHz, DMSO) δ11.23 (s, 1H), 8.82 (d, J=2.7 Hz, 1H), 8.37-8.32 (m, 3H), 8.02 (d, J=2.7 Hz, 1H), 7.91 (s, 1H), 7.87-7.85 (m, 1H), 7.43 (d, J=9.2 Hz, 1H), 7.02 (dd, J=1.8, 9.4 Hz, 1H), 6.68 (dd, J=2.1, 7.4 Hz, 1H), 5.32 (s, 2H), 4.05 (s, 2H), 1.98-1.90 (m, 1H), 0.96-0.91 (m, 2H), 0.72-0.67 (m, 2H).

### Example 33

### Synthesis of 2-(7-chloroimidazo[1,5-a]pyridin-1-yl)-N-(6-((6-cyclopropylimidazo[1,2-a]pyridin-2-yl)methoxy)pyrimidin-4-yl)acetamide (I-33)

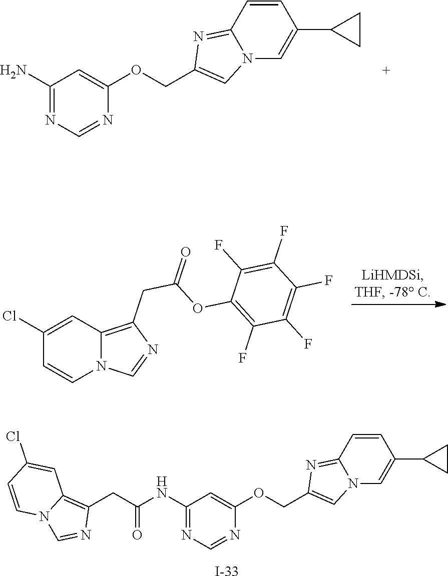

The title compound (6.9 mg, 8%) was prepared using a similar procedure to that used for 2-(7-chloroimidazo[1,5-a]pyridin-1-yl)-N-(5-((6-cyclopropylimidazo[1,2-a]pyridin-2-yl)methoxy)pyridazin-3-yl)acetamide (Example 32) by using 6-((6-cyclopropylimidazo[1,2-a]pyridin-2-yl)methoxy)pyrimidin-4-amine and perfluorophenyl 2-(7-chloroimidazo[1,5-a]pyridin-1-yl)acetate as coupling partners and purified by preparative HPLC. ESI-MS (M+H)⁺: 474.3, ¹H NMR (400 MHz, DMSO) δ11.02 (s, 1H), 8.59 (d, J=1.0 Hz, 1H), 8.35 (d, J=0.6 Hz, 1H), 8.34-8.31 (m, 2H), 7.85 (d, J=0.4 Hz, 1H), 7.85-7.83 (m, 1H), 7.44-7.40 (m, 2H), 7.01 (dd, J=1.8, 9.4 Hz, 1H), 6.67 (dd, J=2.1, 7.4 Hz, 1H), 5.48 (s, 2H), 4.02 (s, 2H), 1.98-1.91 (m, 1H), 0.96-0.90 (m, 2H), 0.71-0.67 (m, 2H).

### Example 34

### Synthesis of 2-(7-chloroimidazo[1,5-a]pyridin-1-yl)-N-(6-((6-cyclopropylimidazo[1,2-a]pyridin-2-yl)methoxy)pyridazin-4-yl)acetamide (I-34)

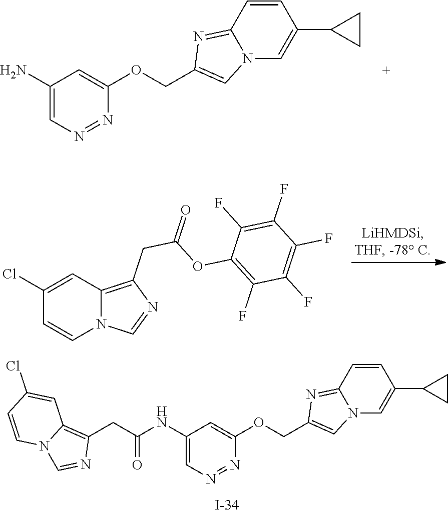

The title compound (5.3 mg, 9%) was prepared using a similar procedure to that used for 2-(7-chloroimidazo[1,5-a]pyridin-1-yl)-N-(5-((6-cyclopropylimidazo[1,2-a]pyridin-2-yl)methoxy)pyridazin-3-yl)acetamide (Example 32) by using 6-((6-cyclopropylimidazo[1,2-a]pyridin-2-yl)methoxy)pyridazin-4-amine and perfluorophenyl 2-(7-chloroimidazo[1,5-a]pyridin-1-yl)acetate as coupling partners and purified by preparative HPLC. ESI-MS (M+H)⁺: 474.2, ¹H NMR (400 MHz, DMSO) δ10.88 (s, 1H), 9.00 (d, J=1.5 Hz, 1H), 8.41-8.37 (m, 3H), 7.92 (s, 1H), 7.86 (s, 1H), 7.52 (d, J=1.3 Hz, 1H), 7.47 (d, J=9.0 Hz, 1H), 7.05 (dd, J=1.5, 9.1 Hz, 1H), 6.72 (dd, J=1.1, 7.6 Hz, 1H), 5.60 (s, 2H), 4.04 (s, 2H), 2.01-1.97 (m, 1H), 1.01-0.95 (m, 2H), 0.76-0.71 (m, 2H).

### Example 35

### Synthesis of 2-(5-chloro-2-(1H-tetrazol-1-yl)phenyl)-N-(6-(((6-cyclopropylimidazo[1,2-a]pyridin-2-yl)methyl)amino)pyrimidin-4-yl)acetamide (I-35)

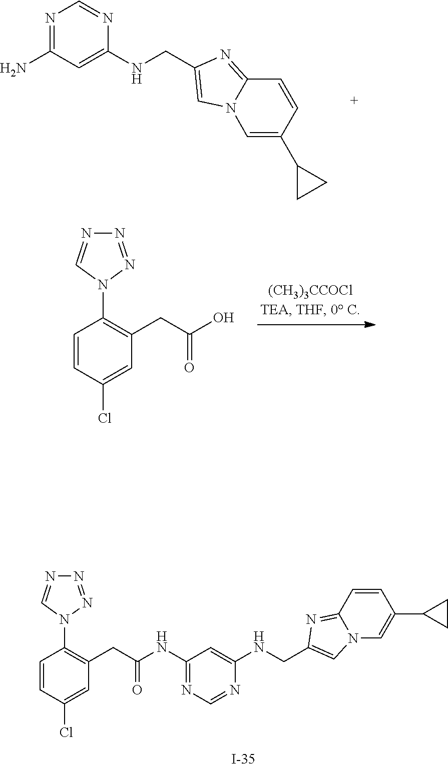

Trimethylacetyl chloride (0.017 mL, 0.13 mmol) was added to a cooled mixture of TEA (0.037 mL, 0.27 mmol) and 2-(5-chloro-2-(1H-tetrazol-1-yl)phenyl)acetic acid (32 mg, 0.134 mmol) in THF (1.5 mL) at 0° C. and the mixture was stirred for 2 h. N₄-((6-cyclopropylimidazo[1,2-a]pyridin-2-yl)methyl)pyrimidine-4,6-diamine (32 mg, 0.11 mmol) was added and the mixture was allowed to warm to room temperature and stirred for 18 h. The mixture was diluted with DCM, washed with Na₂CO₃ (aq. 10%) and concentrated in vacuo. Purification by reverse phase preparative HPLC gave the titled compound. ESI-MS (M+H)⁺: 501.2, ¹H NMR (400 MHz, DMSO) δ10.46-10.41 (m, 1H), 9.83-9.80 (m, 1H), 8.34-8.30 (m, 1H), 8.24-8.20 (m, 1H), 7.87-7.62 (m, 5H), 7.44-7.36 (m, 1H), 7.14-7.08 (m, 1H), 7.01-6.94 (m, 1H), 4.58-4.58 (m, 2H), 3.83-3.78 (m, 2H), 1.97-1.89 (m, 1H), 0.98-0.90 (m, 2H), 0.73-0.65 (m, 2H).

### Example 36

### Synthesis of 2-(6-amino-2-fluoro-3-methoxyphenyl)-N-(6-(((6-cyclopropylimidazo[1,2-a]pyridin-2-yl)methyl)amino)pyrimidin-4-yl)acetamide (I-36)

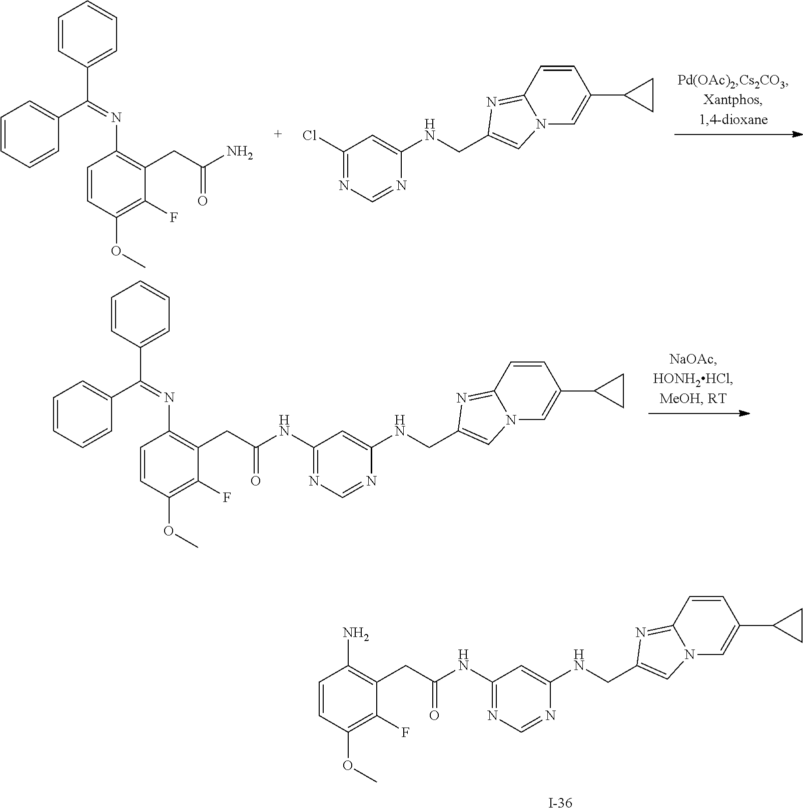

Synthesis of N-(6-(((6-cyclopropylimidazo[1,2-a]pyridin-2-yl)methyl)amino)pyrimidin-4-yl)-2-(6-((diphenylmethylene)amino)-2-fluoro-3-methoxyphenyl)acetamide. Palladium (II) acetate (12 mg, 0.052 mmol) was added to a N₂ purged mixture of 2-(6-((diphenylmethylene)amino)-2-fluoro-3-methoxyphenyl)acetamide (95 mg, 0.26 mmol), 6-chloro-N-((6-cyclopropylimidazo[1,2-a]pyridin-2-yl)methyl)pyrimidin-4-amine (79 mg, 0.26 mmol), Xantphos (61 mg, 0.10 mmol), Cs₂CO₃ (128 mg, 0.393 mmol) in 1,4-dioxane (5 mL) and the reaction heated to 80° C. for 18 h. The mixture was diluted with water (30 mL) and 10% MeOH in DCM (120 mL). The phases were separated with a phase separator cartridge and the organic phase was concentrated in vacuo. Purification by column chromatography on silica gel, eluting with a gradient of 0-1% MeOH in DCM gave the title compound (58 mg, 35%). ¹H NMR (400 MHz, CDCl₃) δ9.94 (s, 1H), 8.34 (s, 1H), 7.94-7.90 (m, 2H), 7.83-7.83 (m, 1H), 7.50-7.30 (m, 9H), 7.16-7.12 (m, 2H), 6.95 (dd, J=1.7, 9.1 Hz, 1H), 6.52 (t, J=8.9 Hz, 1H), 6.06 (dd, J=1.8, 8.8 Hz, 1H), 5.64 (s, 1H), 4.67 (s, 2H), 3.84 (d, J=1.7 Hz, 2H), 3.77 (s, 3H), 1.91-1.84 (m, 1H), 0.98-0.92 (m, 2H), 0.68-0.63 (m, 2H).

Synthesis of 2-(6-amino-2-fluoro-3-methoxyphenyl)-N-(6-(((6-cyclopropylimidazo[1,2-a]pyridin-2-yl)methyl)amino)pyrimidin-4-yl)acetamide. Hydroxylamine (9.2 mg, 0.13 mmol) was added to a mixture of N-(6-(((6-cyclopropylimidazo[1,2-a]pyridin-2-yl)methyl)amino)pyrimidin-4-yl)-2-(6-((diphenylmethylene)amino)-2-fluoro-3-methoxyphenyl)acetamide (46 mg, 0.073 mmol) and sodium acetate (14 mg, 0.176 mmol) in MeOH (1.5 mL) at room temperature. The mixture was stirred for 30 min, diluted with water (6 mL) and extracted with DCM (6 mL). The organic phase was passed through a phase separator cartridge and concentrated in vacuo. The residue was triturated with diethylether to give the title compound (29 mg, 85%). ESI-MS (M+H)⁺: 462.3, ¹H NMR (400 MHz, DMSO) δ10.35 (s, 1H), 8.30 (s, 1H), 8.22 (s, 1H), 7.86 (s, 1H), 7.60 (s, 1H), 7.37 (d, J=9.3 Hz, 1H), 7.20 (s, 1H), 6.96 (dd, J=1.8, 9.3 Hz, 1H), 6.83 (t, J=9.2 Hz, 1H), 6.43 (dd, J=1.4, 8.8 Hz, 1H), 4.86 (s, 2H), 4.57 (s, 2H), 3.71 (s, 3H), 3.66 (s, 2H), 1.95-1.87 (m, 1H), 0.94-0.89 (m, 2H), 0.69-0.64 (m, 2H)

### Example 37

### Synthesis of N-(6-(((6-cyclopropylimidazo[1,2-a]pyridin-2-yl)methyl)amino)pyrimidin-4-yl)-2-(2-fluoro-3-methoxy-6-(1H-tetrazol-1-yl)phenylacetamide formic acid salt (I-37)

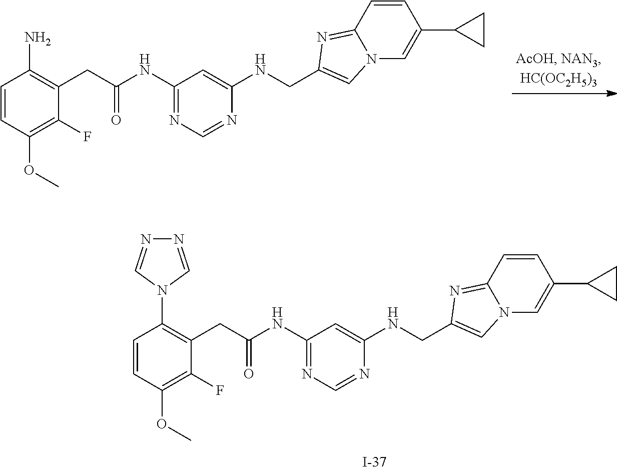

Sodium azide (4.6 mg, 0.07 mmol) was added to a mixture was 2-(6-amino-2-fluoro-3-methoxyphenyl)-N-(6-(((6-cyclopropylimidazo[1,2-a]pyridin-2-yl)methyl)amino)pyrimidin-4-yl)acetamide (26 mg, 0.56 mmol), triethyl orthoformate (0.03 mL, 0.180 mmol) and AcOH (0.5 mL, 0.901 mmol) and stirred for 18 h. The mixture was concentrated in vacuo, the residue was dissolved in 5% MeOH in DCM and washed with water. The phases were separated with a phase separator cartridge and the organic phase was concentrated in vacuo. Purification by reverse phase preparative HPLC gave the title compound (6.2 mg, 21%). ESI-MS (M+H)⁺: 515.4, ¹H NMR (400 MHz, DMSO) δ10.43 (s, 1H), 9.71 (s, 1H), 8.28-8.28 (m, 1H), 8.25 (s, 1H), 8.19-8.18 (m, 1H), 7.83 (br s, 1H), 7.58 (s, 1H), 7.45 (dd, J=1.4, 8.9 Hz, 1H), 7.39-7.33 (m, 2H), 7.08 (s, 1H), 6.95 (dd, J=1.8, 9.3 Hz, 1H), 4.54 (br s, 2H), 3.95 (s, 3H), 3.67 (s, 2H), 1.94-1.86 (m, 1H), 0.93-0.87 (m, 2H), 0.68-0.63 (m, 2H).

### Example 38

### Synthesis of ethyl 3-(2-(((6-(2-(7-chloroimidazo[1,5-a]pyridin-1-yl)acetamido)pyrimidin-4-yl amino methyl)-6-cyclopropylimidazo[1,2-a]pyridin-8-yl)propanoate (I-38)

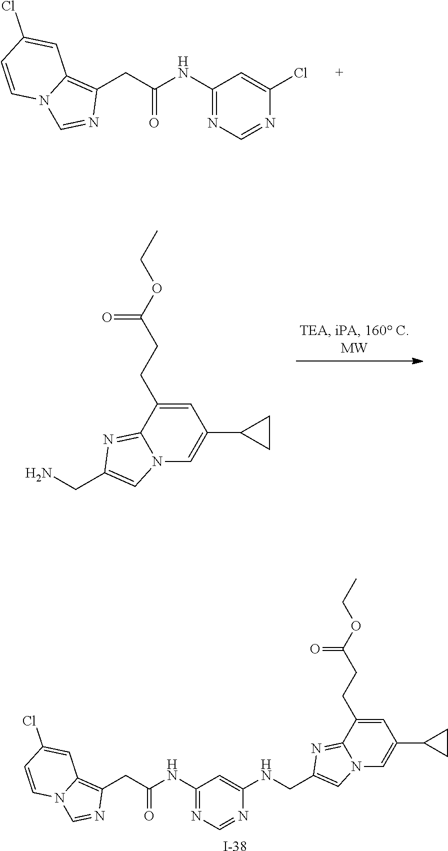

2-(7-Chloroimidazo[1,5-a]pyridin-1-yl)-N-(6-chloropyrimidin-4-yl)acetamide (59 mg, 0.18 mmol) was added to a microwave vial containing ethyl 3-(2-(aminomethyl)-6-cyclopropylimidazo[1,2-a]pyridin-8-yl)propanoate (53 mg, 0.18 mmol), TEA (0.13 mL, 0.92 mmol) and iPrOH (4 mL). The vial was sealed and heated to 160° C. for 15 min in a CEM microwave. The mixture was concentrated in vacuo. Purification by column chromatography on silica gel, eluting with a gradient of 0-20% 7 N NH₃ in MeOH in DCM. Further purification by preparative HPLC gave the title compound (6 mg, 60%) as a yellow oil. ESI-MS (M+H)⁺: 573.4, ¹H NMR (400 MHz, DMSO) δ10.42 (s, 1H), 8.34-8.33 (m, 1H), 8.31 (dd, J=0.9, 7.5 Hz, 1H), 8.20 (d, J=0.8 Hz, 1H), 8.14 (d, J=1.3 Hz, 1H), 7.87-7.81 (m, 2H), 7.56 (s, 1H), 7.22 (s, 1H), 6.77 (d, J=1.4 Hz, 1H), 6.65 (dd, J=2.1, 7.4 Hz, 1H), 4.55 (br s, 2H), 4.05 (q, J=7.1 Hz, 2H), 3.94 (s, 2H), 3.06 (t, J=7.6 Hz, 2H), 2.81-2.75 (m, 2H), 1.89-1.81 (m, 1H), 1.15 (t, J=7.1 Hz, 3H), 0.91-0.85 (m, 2H), 0.65-0.60 (m, 2H).

### Example 39

### Synthesis of 3-(2-(((6-(2-(7-chloroimidazo[1,5-a]pyridin-1-yl)acetamido)pyrimidin-4-yl)amino)methyl)-6-cyclopropylimidazo[1,2-a]pyridin-8-yl)propanoic acid (I-39)

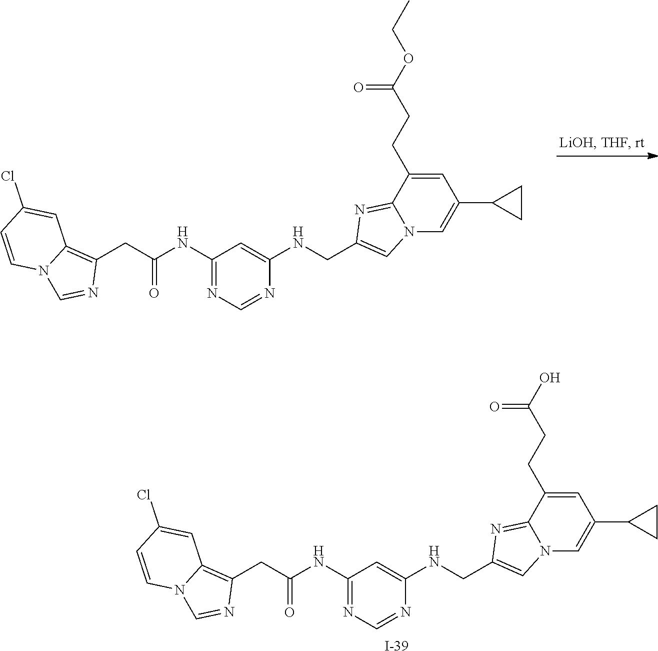

Lithium hydroxide (aq. 1M, 0.087 mL, 0.087 mmol) was added to a solution of ethyl 3-(2-(((6-(2-(7-chloroimidazo[1,5-a]pyridin-1-yl)acetamido)pyrimidin-4-yl)amino)methyl)-6-cyclopropylimidazo[1,2-a]pyridin-8-yl)propanoate (25 mg, 0.043 mmol) in THF (1 mL) and stirred at room temperature for 1 h. The mixture was concentrated in vacuo. Purification by reverse phase preparative HPLC gave the title compound (4 mg, 17%). ESI-MS (M+H)⁺: 545.4, ¹H NMR (400 MHz, DMSO) δ10.42 (s, 1H), 8.34-8.33 (m, 1H), 8.31 (dd, J=0.9, 7.4 Hz, 1H), 8.21-8.19 (m, 1H), 8.12 (d, J=1.3 Hz, 1H), 7.87-7.82 (m, 2H), 7.55 (s, 1H), 7.22 (s, 1H), 6.78-6.77 (m, 1H), 6.65 (dd, J=2.1, 7.5 Hz, 1H), 4.55 (br s, 2H), 3.94 (s, 2H), 3.02 (t, J=7.7 Hz, 2H), 2.63-2.58 (m, 2H), 1.88-1.81 (m, 1H), 0.90-0.85 (m, 2H), 0.65-0.60 (m, 2H). CO₂H proton not observed.

### Example 40

### Synthesis of 3-(2-(((6-(2-(7-chloroimidazo[1,5-a]pyridin-1-yl)acetamido)pyrimidin-4-yl)amino)methyl)-6-cyclopropylimidazo[1,2-a]pyridin-8-yl)-2,2-dimethylpropanoic acid (I-40)

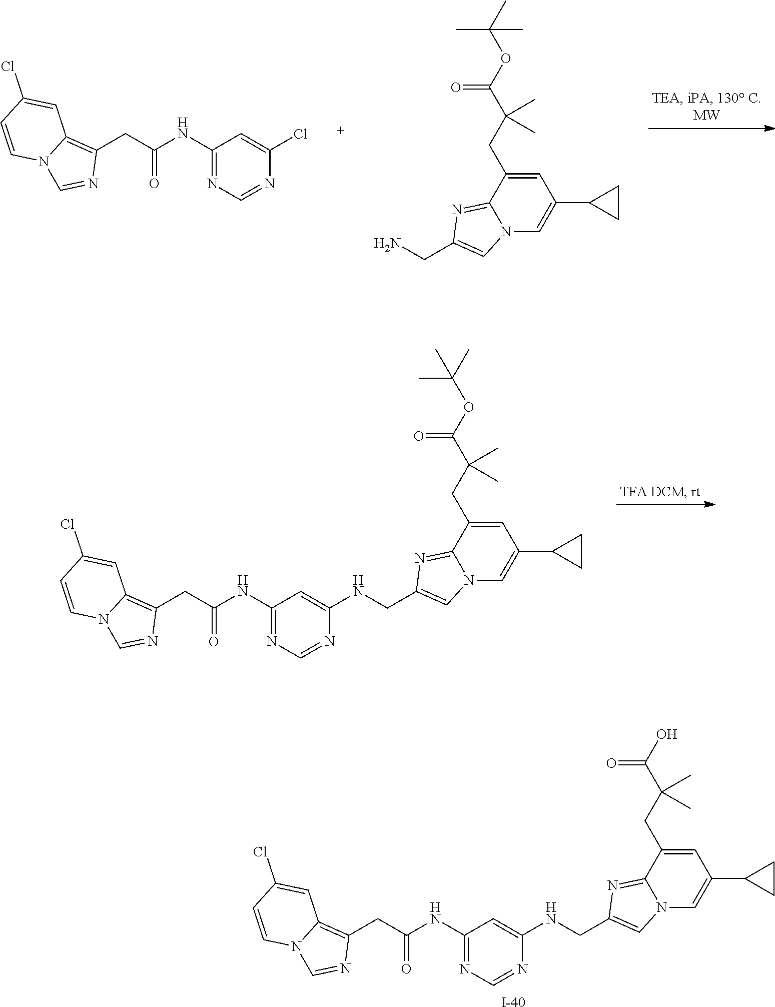

Synthesis of tert-butyl 3-(2-(((6-(2-(7-chloroimidazo[1,5-a]pyridin-1-yl)acetamido)pyrimidin-4-yl)amino)methyl)-6-cyclopropylimidazo[1,2-a]pyridin-8-yl)-2,2-dimethylpropanoate. 2-(7-Chloroimidazo[1,5-a]pyridin-1-yl)-N-(6-chloropyrimidin-4-yl)acetamide (38 mg, 0.12 mmol) was added to a microwave vial containing tert-butyl 3-(2-(aminomethyl)-6-cyclopropylimidazo[1,2-a]pyridin-8-yl)-2,2-dimethylpropanoate (45 mg, 0.13 mmol), TEA (0.049 mL, 0.35 mmol) and iPrOH (1.5 mL). The vial was sealed and heated to 130° C. for 1 h in a CEM microwave. The mixture was concentrated in vacuo. Purification by column chromatography on silica gel, eluting with a gradient of 0-15% MeOH in DCM gave the title compound (33 mg, 44%). ESI-MS (M+H)⁺: 629.4.

Synthesis of 3-(2-(((6-(2-(7-chloroimidazo[1,5-a]pyridin-1-yl)acetamido)pyrimidin-4-yl)amino)methyl)-6-cyclopropylimidazo[1,2-a]pyridin-8-yl)-2,2-dimethylpropanoic acid. Trifluoroacetic acid (0.16 mL, 2.1 mmol) was added to a solution of tert-butyl 3-(2-(((6-(2-(7-chloroimidazo[1,5-a]pyridin-1-yl)acetamido)pyrimidin-4-yl)amino)methyl)-6-cyclopropylimidazo[1,2-a]pyridin-8-yl)-2,2-dimethylpropanoate (33 mg, 0.052 mmol) in DCM (1 mL) and the reaction stirred for 3 h. The mixture was concentrated in vacuo. Purification by reverse phase preparative HPLC gave the title compound (3 mg, 10%). ESI-MS (M+H)⁺: 573.3, ¹H NMR (400 MHz, DMSO) δ12.36 (br s, 1H), 10.46-10.45 (m, 1H), 8.37-8.31 (m, 2H), 8.23-8.16 (m, 2H), 7.88-7.82 (m, 2H), 7.57-7.55 (m, 1H), 7.24 (s, 1H), 6.70-6.66 (m, 2H), 4.57 (s, 2H), 3.97-3.94 (m, 2H), 3.16-3.10 (m, 2H), 1.93-1.80 (m, 1H), 1.15-1.08 (m, 6H), 0.93-0.88 (m, 2H), 0.65-0.59 (m, 2H).

### Example 41

### Synthesis of 2-(7-chloroimidazo[1,5-a]pyridin-1-yl)-N-(4-(((6-cyclopropyl-8-(hydroxymethyl)imidazo[1,2-a]pyridin-2-yl)methyl)amino)pyrimidin-5-yl)acetamide (I-41)

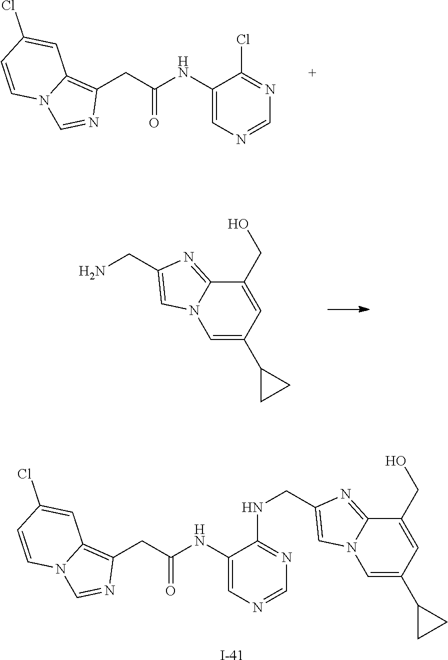

The title compound (12 mg, 38%) was prepared using a similar procedure to that used for the preparation of ethyl 3-(2-(((6-(2-(7-chloroimidazo[1,5-a]pyridin-1-yl)acetamido)pyrimidin-4-yl)amino)methyl)-6-cyclopropylimidazo[1,2-a]pyridin-8-yl)propanoate (Example 38) by using 2-(7-chloroimidazo[1,5-a]pyridin-1-yl)-N-(4-chloropyrimidin-5-yl)acetamide and (2-(aminomethyl)-6-cyclopropylimidazo[1,2-a]pyridin-8-yl)methanol as coupling partners, followed by purification by preparative HPLC. ESI-MS (M+H)⁺: 503.3, ¹H NMR (400 MHz, DMSO) δ9.56 (s, 1H), 8.32 (s, 2H), 8.28 (d, J=7.6 Hz, 1H), 8.17-8.13 (m, 2H), 7.79 (s, 1H), 7.68 (t, J=5.6 Hz, 1H), 7.63 (s, 1H), 6.97 (s, 1H), 6.65 (dd, J=1.9, 7.5 Hz, 1H), 5.31 (s, 1H), 4.78 (s, 2H), 4.72-4.67 (m, 2H), 3.91 (s, 2H), 1.96-1.87 (m, 1H), 0.94-0.87 (m, 2H), 0.68-0.61 (m, 2H).

The compounds in the table below were prepared using a similar procedure to that used for the preparation of 2-(7-chloroimidazo[1,5-a]pyridin-1-yl)-N-(4-(((6-cyclopropyl-8-(hydroxymethyl)imidazo[1,2-a]pyridin-2-yl)methyl)amino)pyrimidin-5-yl)acetamide (Example 41) using 2-(7-chloroimidazo[1,5-a]pyridin-1-yl)-N-(4-chloropyrimidin-5-yl)acetamide and an appropriate coupling partner.

| EG | Compound | Coupling Partner | Analytical Data |
|---|---|---|---|
| 42 | 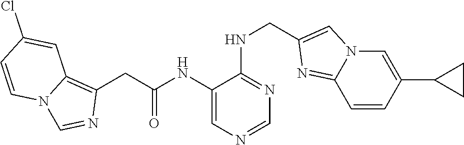 [SMILES: O=C(Cc1ncn2ccc(Cl)cc12)Nc1cncnc1NCc1cn2cc(C3CC3)ccc2n1] 2-(7-chloroimidazo[1,5-a]pyridin-1-yl)-N-(4-(((6- cyclopropylimidazo[1,2-a]pyridin-2-yl)methyl)amino) pyrimidin-5-yl)acetamide | (6-cyclo propyl imidazo[1,2-a] pyridin-2-yl) methanamine | ESI-MS (M + H)⁺: 473.3, , ¹H NMR (400 MHz, DMSO) δ 9.55 (s, 1H), 8.34-8.25 (m, 4H), 8.14 (s, 1H), 7.79 (s, 1H), 7.66-7.62 (m, 2H), 7.39 (d, J = 9.3 Hz, 1H), 6.96 (dd, J = 1.3, 9.3 Hz, 1H), 6.67-6.62 (m, 1H), 4.70 (d, J = 5.3 Hz, 2H), 3.91 (s, 2H), 1.95- 1.86 (m, 1H), 0.94-0.86 (m, 2H), 0.69-0.62 (m, 2H). |
| 43 | 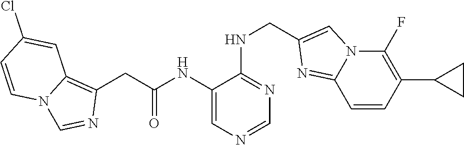 [SMILES: O=C(Cc1ncn2ccc(Cl)cc12)Nc1cncnc1NCc1cn2c(F)c(C3CC3)ccc2n1] 2-(7-chloroimidazo[1,5-a]pyridin-1-yl)-N-(4-(((6- cyclopropyl-5-fluoroimidazo[1,2-a]pyridin-2- yl)methyl)amino)pyrimidin-5-yl)acetamide (I-43) | (6-cyclopropyl- 5-fluoroimidazo [1,2-a]pyridin-2-yl) methanamine | ESI-MS (M + H)⁺: 491.3, , ¹H NMR (400 MHz, CDCl₃) δ 9.07 (s, 1H), 8.51 (s, 1H), 8.29 (s, 1H), 8.03 (s, 1H), 7.77 (d, J = 7.8 Hz, 1H), 7.56 (s, 1H), 7.42 (s, 1H), 7.23 (m, 1H), 6.85 (dd, J = 8.6, 8.6 Hz, 1H), 6.61 (dd, J = 5.2, 5.2 Hz, 1H), 6.53 (dd, J = 1.9, 7.4 Hz, 1H), 4.84 (d, J = 5.4 Hz, 2H), 3.90 (s, 2H), 1.05-0.99 (m, 2H), 0.75-0.70 (m, 2H). One proton obscured by H₂O signal. |

### Example 44

### Synthesis of 2-(7-chloroimidazo[-1,5-a]pyridin-1-yl)-N-(6-(((6-cyclopropyl-8-(hydroxymethyl)imidazo[1,2-a]pyridin-2-yl)methyl)amino)pyrimidin-4-yl)acetamide formic acid salt (I-44)

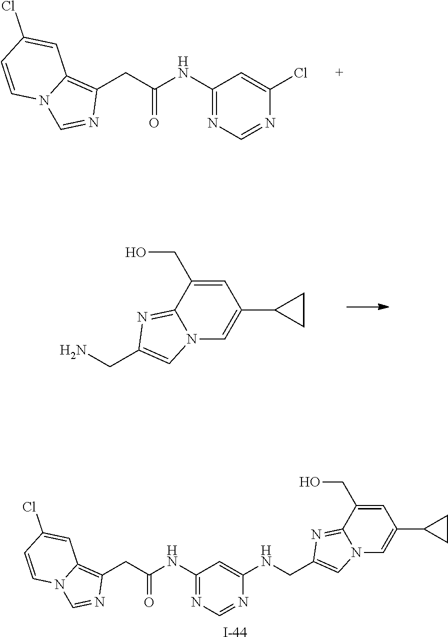

The title compound (3.8 mg, 240%) was prepared using a similar procedure to that used for the preparation of ethyl 3-(2-(((6-(2-(7-chloroimidazo[1,5-a]pyridin-1-yl)acetamido)pyrimidin-4-yl)amino)methyl)-6-cyclopropylimidazo[1,2-a]pyridin-8-yl)propanoate (Example 38) by using 2-(7-chloroimidazo[1,5-a]pyridin-1-yl)-N-(6-chloropyrimidin-4-yl)acetamide and (2-(aminomethyl)-6-cyclopropylimidazo[1,2-a]pyridin-8-yl)methanol as coupling partners and purified by preparative HPLC. ESI-MS (M+H)⁺: 503.3, ¹H NMR (400 MHz, DMSO) δ10.42 (s, 1H), 8.47 (s, 0.4H), 8.35-8.29 (m, 2H), 8.21-8.19 (m, 1H), 8.18-8.15 (m, 1H), 7.84-7.81 (m, 2H), 7.58 (s, 1H), 7.23-7.22 (m, 1H), 6.97-6.94 (m, 1H), 6.65 (dd, J=1.9, 7.5 Hz, 1H), 5.30-5.27 (m, 1H), 4.77-4.73 (m, 2H), 4.54 (br s, 2H), 3.94 (s, 2H), 1.95-1.87 (m, 1H), 0.93-0.87 (m, 2H), 0.66-0.60 (in, 2H).

The compounds in the table below were prepared using a similar procedure to that used for the preparation of 2-(7-chloroimidazo[1,5-a]pyridin-1-yl)-N-(6-(((6-cyclopropyl-8-(hydroxymethyl)imidazo[1,2-a]pyridin-2-yl)methyl)amino)pyrimidin-4-yl)acetamide (Example 44), using 2-(7-chloroimidazo[1,5-a]pyridin-1-yl)-N-(6-chloropyrimidin-4-yl)acetamide and an appropriate coupling partner then purified by preparative HPLC.

| | | Coupling | |
|---|---|---|---|
| EG | Compound | Partner | Analytical Data |
| 45 | 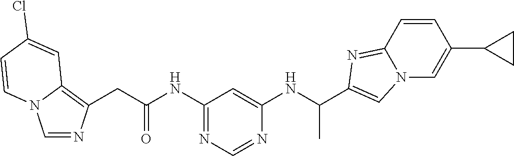 [SMILES: CC(Nc1cc(NC(=O)Cc2ncn3ccc(Cl)cc23)ncn1)c1cn2cc(C3CC3)ccc2n1] 2-(7-chloroimidazo[1,5-a]pyridin-1-yl)-N-(6-((1-(6-cyclopropyl imidazo[1,2- a]pyridin-2-yl)ethyl)amino)pyrimidin-4-yl)acetamide (I-45) | 1-(6-cyclo- propyl- imidazo [1,2-a] pyridin- 2-yl)ethan-1- amine | ESI-MS (M + H)⁺: 487.3, ¹H NMR (400 MHz, CDCl₃) δ 9.41 (s, 1H), 8.27 (s, 1H), 8.13 (s, 1H), 7.84-7.81 (m, 2H), 7.47-7.39 (m, 4H), 7.30 (s, 1H), 6.95-6.90 (m, 1H), 6.54 (dd, J = 1.9, 7.5 Hz, 1H), 5.72 (d, J = 6.9 Hz, 1H), 3.91-3.90 (m, 2H), 1.91- 1.81 (m, 1H), 1.65 (d, J = 6.8 Hz, 3H), 0.98-0.91 (m, 2H), 0.68-0.62 (m, 2H). |
| 46 | 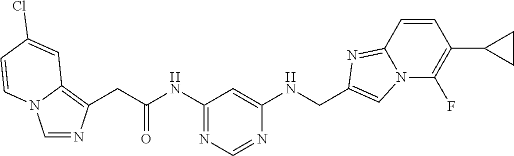 [SMILES: O=C(Cc1ncn2ccc(Cl)cc12)Nc1cc(NCc2cn3c(F)c(C4CC4)ccc3n2)ncn1] 2-(7-chloroimidazo[1,5-a]pyridin-1-yl)-N-(6-(((6-cyclopropyl-5-fluoro- imidazo[1,2-a]pyridin-2-yl)methyl)amino)pyrimidin-4-yl)acetamide (I-46) | (6-cyclo- propyl-5- fluoro- imidazo [1,2-a] pyridin- 2-yl) methanamine | ESI-MS (M + H)⁺: 491.2, ¹H NMR (400 MHz, CDCl₃) δ 9.43 (s, 1H), 8.32 (s, 1H), 8.14 (s, 1H), 7.83 (d, J = 7.3 Hz, 1H), 7.55 (s, 1H), 7.41 (s, 1H), 7.35 (s, 1H), 7.32-7.28 (m, 1H), 6.86 (t, J = 8.6 Hz, 1H), 6.57-6.52 (m, 1H), 5.64 (s, 1H), 4.70 (s, 2H), 3.92 (s, 2H), 2.05-1.98 (m, 1H), 1.05- 0.97 (m, 2H), 0.75-0.70 (m, 2H). |
| 47 | 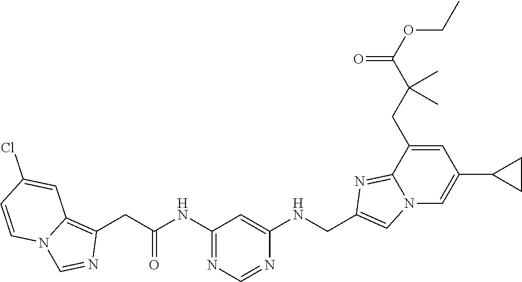 [SMILES: CCOC(=O)C(C)(C)Cc1cc(C2CC2)cn2cc(CNc3cc(NC(=O)Cc4ncn5ccc(Cl)cc45)ncn3)nc12] ethyl 3-(2-(((6-(2-(7-chloroimidazo[1,5-a]pyridin-1-yl)acetamido)pyrimidin-4-yl) amino)methyl)-6-cyclopropylimidazo[1,2-a]pyridin-8-yl)-2,2- dimethylpropanoate (I-47) | ethyl 3-(2- (amino- methyl)- 6-cyclo propyl- imidazo [1,2-a] pyridin- 8-yl)-2,2- dimethyl propanoate formic acid salt | ESI-MS (M + H)⁺: 601.5, ¹H NMR (400 MHz, DMSO) δ 10.43 (s, 1H), 8.35-8.29 (m, 4H), 8.19 (s, 1H), 8.15 (d, J = 1.0 Hz, 1H), 7.83-7.81 (m, 1H), 7.54 (s, 1H), 7.23 (s, 1H), 6.65 (q, J = 3.2 Hz, 1H), 6.59 (d, J = 1.1 Hz, 1H), 4.54 (br s, 2H), 4.01 (q, J = 7.0 Hz, 2H), 3.94 (s, 2H), 3.10 (s, 2H), 1.90-1.81 (m, 1H), 1.15-0.09 (m, 9H), 0.91-0.86 (m, 2H), 0.60-0.55 (m, 2H). |

### Example 48

### Synthesis of 2-(7-chloroimidazo[1,5-a]pyridin-1-yl)-N-(6-(((6-cyclopropyl-8-(morpholinomethyl)imidazo[1,2-a]pyridin-2-yl)methyl)amino)pyrimidin-4-yl)acetamide (I-48)

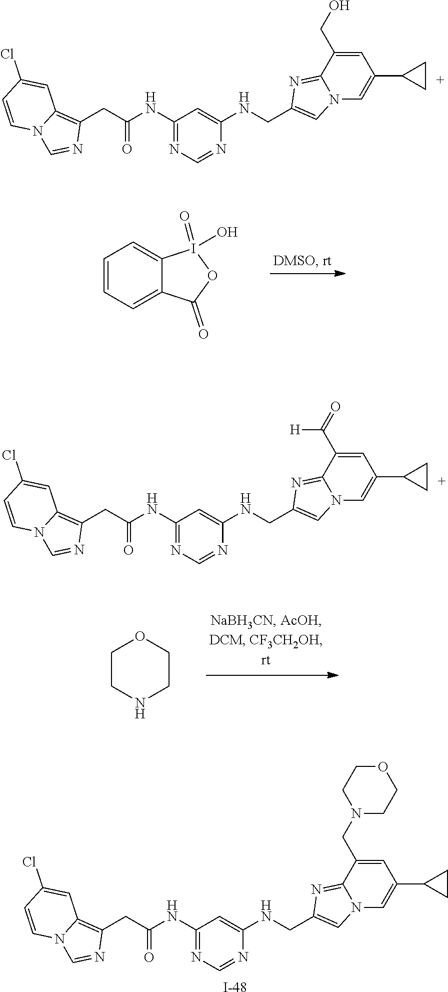

Synthesis of 2-(7-chloroimidazo[1,5-a]pyridin-1-yl)-N-(6-(((6-cyclopropyl-8-formylimidazo[1,2-a]pyridin-2-yl)methyl)amino)pyrimidin-4-yl)acetamide. 2-Iodooxybenzoic acid (52 mg, 0.083 mmol), was added to a solution of 2-(7-chloroimidazo[1,5-a]pyridin-1-yl)-N-(6-(((6-cyclopropyl-8-(hydroxymethyl)imidazo[1,2-a]pyridin-2-yl)methyl)amino)pyrimidin-4-yl)acetamide (35 mg, 0.069 mmol) in DMSO (1 mL) and stirred at room temperature for 2 h. The reaction was quenched with Na₂S₂O₃ (aq. sat.) followed by NaHCO₃ (aq. sat. 15 mL), water (10 mL) and extracted with DCM (3×25 mL). The organic phases were combined, passed through a phase separator cartridge and concentrated in vacuo to give the crude title compound which was taken on to the next step without further purification. ESI-MS (M+H)⁺: 501.3

Synthesis of 2-(7-chloroimidazo[1,5-a]pyridin-1-yl)-N-(6-(((6-cyclopropyl-8-(morpholinomethyl)imidazo[1,2-a]pyridin-2-yl)methyl)amino)pyrimidin-4-yl)acetamide. Sodium cyanoborohydride (8.8 mg, 0.14 mmol) was added to a mixture of 2-(7-chloroimidazo[1,5-a]pyridin-1-yl)-N-(6-(((6-cyclopropyl-8-formylimidazo[1,2-a]pyridin-2-yl)methyl)amino)pyrimidin-4-yl)acetamide (35 mg, 0.067 mmol), morpholine (0.031 mL, 0.349 mmol) and acetic acid (0.008 mL, 0.14 mmol), in 2,2,2-trifluoroethanol (2 mL), DCM (0.5 mL) and stirred at room temperature for 96 h. Additional amounts of sodium cyanoborohydride (8.8 mg, 0.14 mmol), morpholine (0.031 mL, 0.349 mmol) and acetic acid (0.008 mL, 0.14 mmol) were added after 48 h. The mixture was diluted with NaHCO₃ (aq. sat. 15 mL), water (15 mL) and extracted with EtOAc (2×25 mL), DCM (25 mL). The organics were combined, passed through a phase separator cartridge and concentrated in vacuo. Purification by reverse phase preparative HPLC gave the title compound (6.1 mg, 15%) as a brown solid. ESI-MS (M+H)⁺: 572.4, ¹H NMR (400 MHz, DMSO) δ10.43 (s, 1H), 8.34-8.29 (m, 2H), 8.20-8.16 (m, 2H), 7.86-7.80 (m, 2H), 7.57 (s, 1H), 7.23-7.22 (m, 1H), 6.96-6.94 (m, 1H), 6.65 (dd, J=2.1, 7.5 Hz, 1H), 4.55 (br s, 2H), 3.94 (s, 2H), 3.74 (s, 2H), 3.59 (t, J=4.6 Hz, 4H), 2.46-2.41 (m, 4H), 1.94-1.87 (m, 1H), 0.93-0.87 (m, 2H), 0.65-0.60 (m, 2H).

### Example 49

### Synthesis of 2-(3-chlorophenoxy)-N-(6-(((6-cyclopropylimidazo[1,2-a]pyridin-2-yl)methyl)amino)pyrimidin-4-yl)propanamide formic acid salt (I-49)

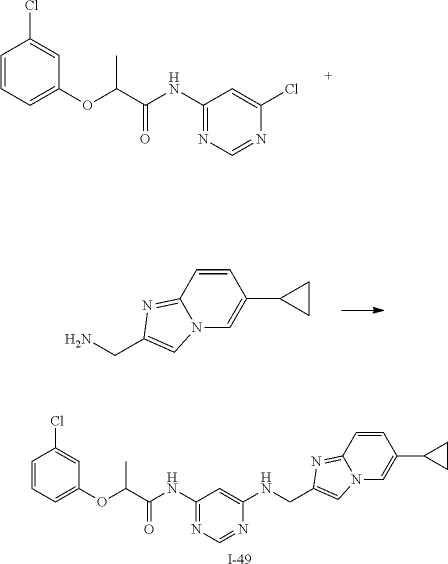

The title compound (49 mg, 44%) was prepared using a similar procedure to that used for ethyl 3-(2-(((6-(2-(7-chloroimidazo[1,5-a]pyridin-1-yl)acetamido)pyrimidin-4-yl)amino)methyl)-6-cyclopropylimidazo[1,2-a]pyridin-8-yl)propanoate (Example 38) by using 2-(3-chlorophenoxy)-N-(6-chloropyrimidin-4-yl)propanamide and (6-cyclopropylimidazo[1,2-a]pyridin-2-yl)methanamine hydrochloride as coupling partners and purified by preparative HPLC. ESI-MS (M+H)⁺: 463.4, ¹H NMR (400 MHz, DMSO) δ10.54 (s, 1H), 8.28-8.26 (m, 1H), 8.23 (d, J=0.7 Hz, 1H), 8.20 (s, 0.33H), 7.92 (br s, 1H), 7.60 (d, J=0.4 Hz, 1H), 7.37-7.29 (m, 2H), 7.22 (br s, 1H), 7.02-6.98 (m, 2H), 6.95 (dd, J=1.8, 9.3 Hz, 1H), 6.90-6.86 (m, 1H), 5.07 (q, J=6.5 Hz, 1H), 4.57 (br s, 2H), 1.94-1.85 (m, 1H), 1.50 (d, J=6.5 Hz, 3H), 0.93-0.87 (m, 2H), 0.67-0.62 (m, 2H).

### Example 50

### Synthesis of (E)-3-(3-chlorophenyl)-N-(6-(((6-cyclopropylimidazo[1,2-a]pyridin-2-yl)methyl)amino)pyrimidin-4-yl)acrylamide (I-50)

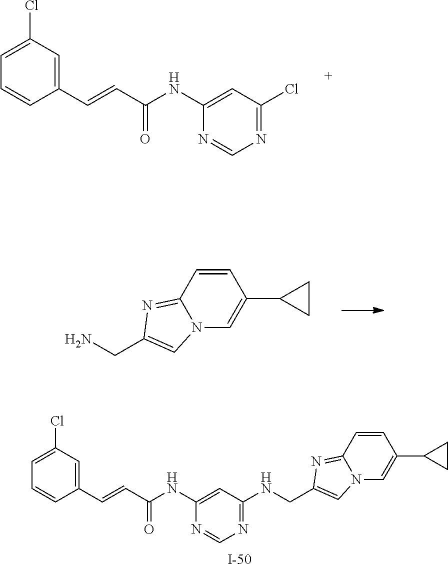

The title compound (49 mg, 44%) was prepared using a similar procedure to that used for ethyl 3-(2-(((6-(2-(7-chloroimidazo[1,5-a]pyridin-1-yl)acetamido)pyrimidin-4-yl)amino)methyl)-6-cyclopropylimidazo[1,2-a]pyridin-8-yl)propanoate (Example 38) by using (E)-3-(3-chlorophenyl)-N-(6-chloropyrimidin-4-yl)acrylamide and (6-cyclopropylimidazo[1,2-a]pyridin-2-yl)methanamine hydrochloride as coupling partners and purified by preparative HPLC. ESI-MS (M+H)⁺: 445.4, ¹H NMR (400 MHz, DMSO) δ10.49 (s, 1H), 8.32 (s, 1H), 8.25 (s, 1H), 7.96-7.96 (m, 1H), 7.68 (s, 1H), 7.63 (d, J=7.3 Hz, 1H), 7.59 (dd, J=3.6, 3.6 Hz, 2H), 7.50 (d, J=5.1 Hz, 2H), 7.44-7.37 (m, 2H), 7.09 (d, J=4.5 Hz, 1H), 6.97 (dd, J=1.8, 9.4 Hz, 1H), 4.61-4.60 (m, 2H), 1.96-1.88 (m, 1H), 0.95-0.89 (m, 2H), 0.70-0.65 (m, 2H).

### Example 51

### Synthesis of 2-(2-bromo-5-chlorophenyl)-N-(6-(((6-cyclopropylimidazo[1,2-a]pyridin-2-yl)methyl)amino)pyrimidin-4-yl)acetamide (I-51)

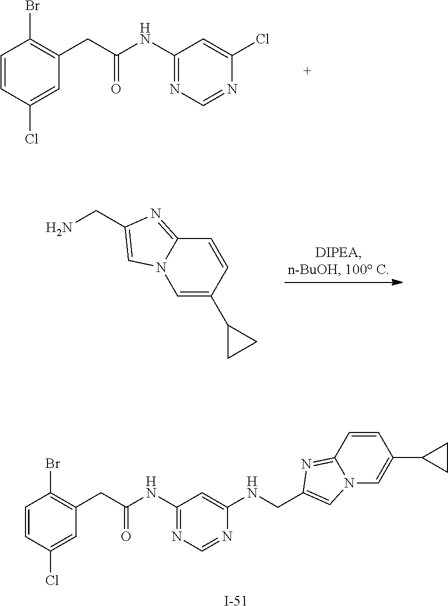

(6-Cyclopropylimidazo[1,2-a]pyridin-2-yl)methanamine (16 mg, 0.083 mmol) was added to a mixture of 2-(2-bromo-5-chlorophenyl)-N-(6-chloropyrimidin-4-yl)acetamide (25 mg, 0.0692 mmol), DIPEA (0.024 mL, 0.138 mmol) in n-butanol (1 mL) and heated to 100° C. for 18 h. The mixture was diluted with water (5 mL), extracted with DCM (3×5 mL), the organic phases were combined, passed through a hydrophobic cartridge and concentrated in vacuo. Purification by column chromatography on silica gel, eluting with a gradient of 0-10% MeOH in DCM gave the title compound (20 mg, 56%). ESI-MS (M+H)⁺: 511.2, ¹H NMR (400 MHz, DMSO) δ10.61 (s, 1H), 8.30 (s, 1H), 8.23 (s, 1H), 7.86 (s, 1H), 7.66-7.60 (m, 2H), 7.54 (d, J=2.5 Hz, 1H), 7.37 (d, J=9.5 Hz, 1H), 7.32 (dd, J=2.8, 8.9 Hz, 1H), 7.19 (s, 1H), 6.97 (dd, J=2.0, 9.1 Hz, 1H), 4.57 (s, 2H), 3.94 (s, 2H), 1.95-1.88 (m, 1H), 0.95-0.89 (m, 2H), 0.69-0.65 (m, 2H).

### Example 52

### Synthesis of 2-(7-chloroimidazo[1,5-a]pyridin-1-yl)-N-(4-(((6-cyclopropyl-8-(hydroxymethyl)imidazo[1,2-a]pyridin-2-yl)methyl)amino)pyridin-2-yl)acetamide (I-52)

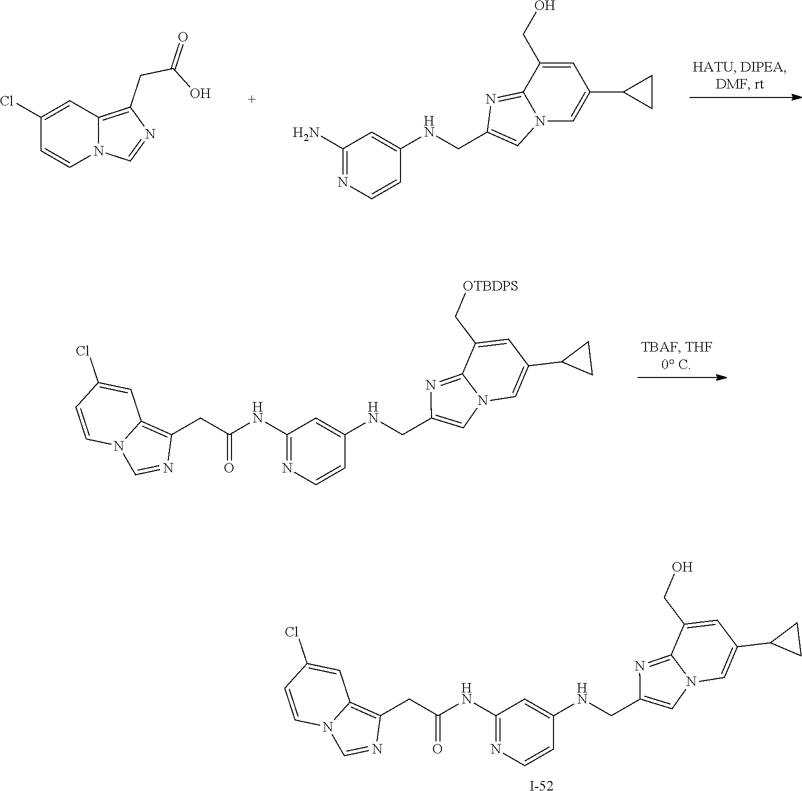

Synthesis of N-(4-(((8-(((tert-butyldiphenylsilyl)oxy)methyl)-6-cyclopropylimidazo[1,2-a]pyridin-2-yl)methyl)amino)pyridin-2-yl)-2-(7-chloroimidazo[1,5-a]pyridin-1-yl)acetamide. N⁴-((8-(((tert-butyldiphenylsilyl)oxy)methyl)-6-cyclopropylimidazo[1,2-a]pyridin-2-yl)methyl)pyridine-2,4-diamine (150 mg, 0.268 mmol) was added to a mixture of 2-(7-chloroimidazo[1,5-a]pyridin-1-yl)acetic acid (57 mg, 0.268 mmol), DIPEA (0.093 mL, 0.537 mmol) and HATU (110 mg, 0.295 mmol) in DMF (2.5 mL) and stirred at room temperature for 3 h. The mixture was diluted with water (25 mL) and brine (25 mL) and extracted with EtOAc (3×50 mL). The organic phases were combined and concentrated in vacuo. Purification by column chromatography on silica gel, eluting with a gradient of 0-20% 7N NH₃/MeOH in DCM gave the title compound (74 mg, 36%) as a yellow solid. ESI-MS (M+H)⁺: 740.5

Synthesis of 2-(7-chloroimidazo[1,5-a]pyridin-1-yl)-N-(4-(((6-cyclopropyl-8-(hydroxymethyl)imidazo[1,2-a]pyridin-2-yl)methyl)amino)pyridin-2-yl)acetamide. TBAF (1M in THF, 0.099 mL, 0.099 mmol) was added to a cooled solution of N-(4-(((8-(((tert-butyldiphenylsilyl)oxy)methyl)-6-cyclopropylimidazo[1,2-a]pyridin-2-yl)methyl)amino)pyridin-2-yl)-2-(7-chloroimidazo[1,5-a]pyridin-1-yl)acetamide (974 mg, 0.099 mmol) in THF (3 mL) at 0° C. and stirred for 3 h. The mixture was diluted with water (25 mL) and brine (25 mL) and extracted with EtOAc (3×50 mL). The organic phases were combined, dried (MgSO₄), filtered and concentrated in vacuo. Purification by reverse phase preparative HPLC gave the title compound (17 mg, 34%). ESI-MS (M+H)⁺: 502.3, ¹H NMR (400 MHz, DMSO) δ10.16 (br s, 1H), 8.34-8.33 (m, 1H), 8.31 (dd, J=0.9, 7.5 Hz, 1H), 8.18-8.17 (m, 1H), 7.84-7.82 (m, 1H), 7.76 (d, J=5.9 Hz, 1H), 7.61 (s, 1H), 7.36-7.13 (m, 2H), 6.98-6.96 (m, 1H), 6.65 (dd, J=2.1, 7.5 Hz, 1H), 6.35-6.31 (m, 1H), 5.29 (t, J=5.7 Hz, 1H), 4.76 (d, J=5.6 Hz, 2H), 4.36-4.32 (m, 2H), 3.91 (s, 2H), 1.95-1.87 (m, 1H), 0.93-0.87 (m, 2H), 0.66-0.61 (m, 2H).

### Example 53

### Synthesis of 2-(7-bromo-8-fluoroimidazo[1,5-a]pyridin-1-yl)-N-(6-(((6-cyclopropylimidazo[1,2-a]pyridin-2-yl)methyl)amino)pyrimidin-4-yl)acetamide (I-53)

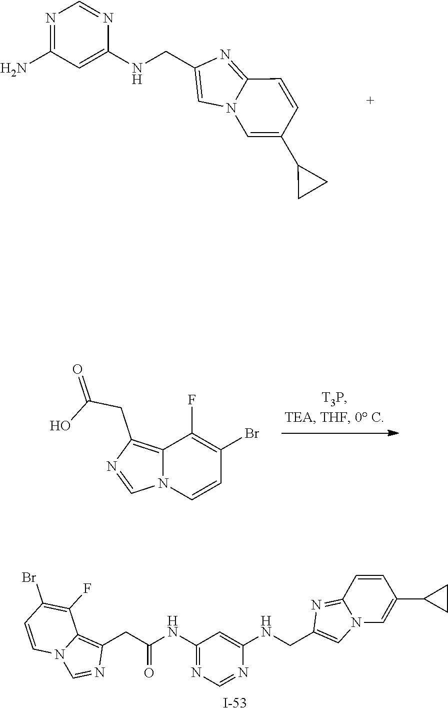

Propylphosphonic anhydride solution (50%, 218 μL, 0.36 mmol), was added dropwise to a mixture of N⁴-((6-cyclopropylimidazo[1,2-a]pyridin-2-yl)methyl)pyrimidine-4,6-diamine (37 mg, 0.13 mmol), 2-(7-bromo-8-fluoroimidazo[1,5-a]pyridin-1-yl)acetic acid (40 mg, 0.15 mmol), TEA (71 μL, 0.51 mmol) in THF (2 mL) at room temperature and stirred for 2 h. The mixture was concentrated in vacuo, the residue was dissolved in EtOAc, washed with NaHCO₃ (aq. sat.), passed through a phase separator cartridge and concentrated in vacuo. Purification by reverse phase preparative HPLC gave the titled compound (7 mg, 10%). ESI-MS (M+H)⁺: 535.4/537.4, ¹H NMR (400 MHz, DMSO) δ10.46 (s, 1H), 8.47-8.43 (m, 1H), 8.29 (s, 1H), 8.21 (s, 1H), 8.14 (d, J=7.3 Hz, 1H), 7.83 (s, 1H), 7.59 (s, 1H), 7.38-7.32 (m, 1H), 7.22 (s, 1H), 6.97-6.93 (m, 1H), 6.81 (t, J=6.8 Hz, 1H), 4.55 (s, 2H), 4.00 (s, 2H), 1.94-1.85 (m, 1H), 0.93-0.86 (m, 2H), 0.68-0.61 (m, 2H).

### Example 54

### Synthesis of 2-(7-chloroimidazo[1,5-a]pyridin-1-yl)-N-(6-(((6-cyclopropylimidazo[1,2-a]pyridin-2-yl)methy)amino)pyrimidin-4-yl)-2-oxoacetamide (I-54)

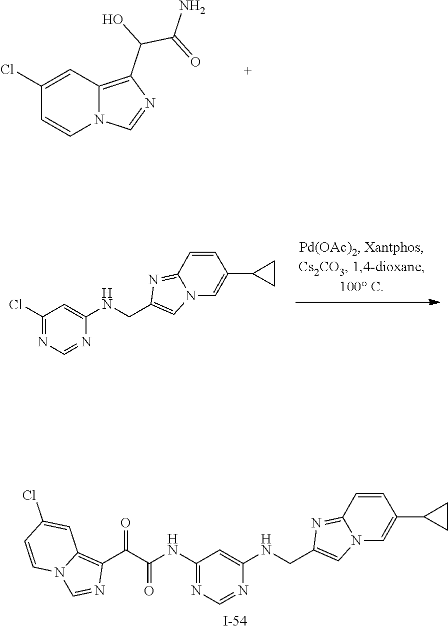

A suspension of 6-chloro-N-((6-cyclopropylimidazo[1,2-a]pyridin-2-yl)methyl)pyrimidin-4-amine (79 mg, 0.26 mmol), 2-(7-chloroimidazo[1,5-a]pyridin-1-yl)-2-hydroxyacetamide (79 mg, 0.35 mmol), Cs₂CO₃ (170 mg, 0.53 mmol), Xantphos (81 mg, 0.14 mmol) and Pd(OAc)₂ (16 mg, 0.070 mmol) in anhydrous 1,4-dioxane (2.0 mL) was degassed for 10 minutes and the reaction mixture was then stirred at 100° C. for 4 hours. The reaction mixture was allowed to cool to room temperature and was concentrated in vacuo. The residue was suspended in water (10 mL), filtered and washed with water. The collected solid was dried in vacuo, suspended in 5% MeOH in DCM, stirred for 5 minutes, filtered and the filtrate was concentrated in vacuo. The residue was purified by column chromatography on silica gel, eluting with a gradient of 1-5% 7 N NH₃ in MeOH in DCM. The residue was further purified by preparative HPLC to give the title compound (3 mg, 2%) as a yellow solid. ESI-MS (M+H)⁺: 487.3, ¹H NMR (400 MHz, DMSO) δ11.17 (s, 1H), 8.67 (d, J=7.2 Hz, 1H), 8.59-8.54 (m, 1H), 8.45 (s, 1H), 8.24 (s, 1H), 8.18 (s, 1H), 8.11 (s, 1H), 7.95-7.94 (m, 1H), 7.58 (s, 1H), 7.32 (d, J=9.2 Hz, 1H), 7.18 (d, J=6.7 Hz, 1H), 6.90 (dd, J=1.7, 9.3 Hz, 1H), 4.55-4.55 (m, 2H), 1.88-1.80 (m, 1H), 0.87-0.81 (m, 2H), 0.62-0.56 (m, 2H).

### Example 55

### Synthesis of N⁴-((6-cyclopropylimidazo[1,2-a]pyridin-2-yl)methyl)-N⁶-(2-fluoro-3-methoxy-6-(1H-tetrazol-1-yl)benzyl)pyrimidine-4,6-diamine (I-55)

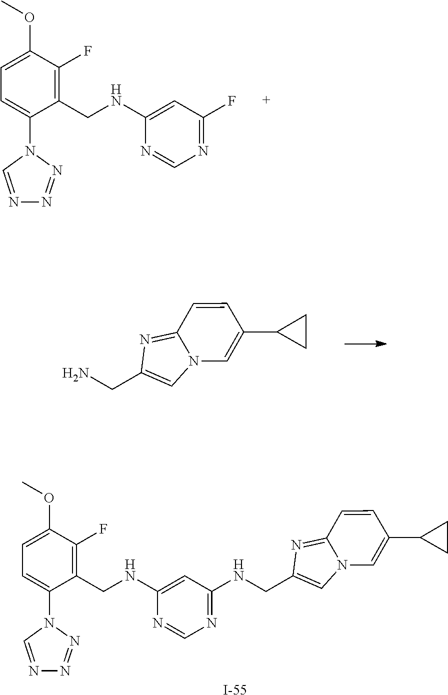

The title compound (49 mg, 44%) was prepared using a similar procedure to that used for 2-(2-bromo-5-chlorophenyl)-N-(6-(((6-cyclopropylimidazo[1,2-a]pyridin-2-yl)methyl)amino)pyrimidin-4-yl)acetamide (Example 51) by using 6-fluoro-N-(2-fluoro-3-methoxy-6-(1H-tetrazol-1-yl)benzyl)pyrimidin-4-amine and (6-cyclopropylimidazo[1,2-a]pyridin-2-yl)methanamine as coupling partners and purified by preparative HPLC. ESI-MS (M+H)⁺: 487.4, ¹H NMR (400 MHz, DMSO) δ 9.73 (s, 1H), 8.29-8.28 (m, 1H), 7.74 (s, 1H), 7.54 (s, 1H), 7.40-7.32 (m, 3H), 7.02 (t, J=5.8 Hz, 1H), 6.98-6.91 (m, 2H), 5.38 (s, 1H), 4.40 (d, J=5.4 Hz, 2H), 4.24 (d, J=4.6 Hz, 2H), 3.94 (s, 3H), 1.95-1.87 (m, 1H), 0.94-0.88 (m, 2H), 0.69-0.64 (m, 2H).

### Example 56

### Synthesis of N⁵-(6-(((6-cyclopropylimidazo[1,2-a]pyridin-2-yl)methyl)amino)pyrimidin-4-yl)-6,7-dihydro-5H-cyclopenta[c]pyridine-1,5-diamine (I-56)

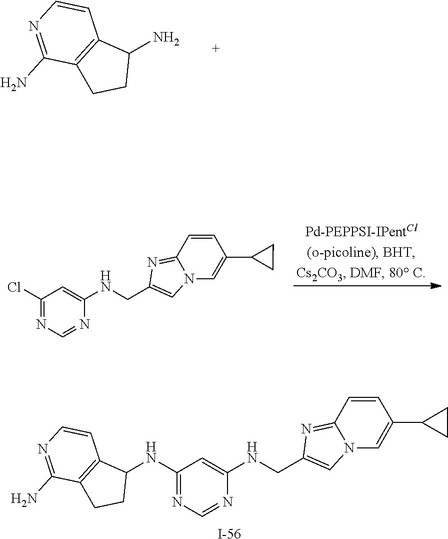

A mixture of 6-chloro-N-((6-cyclopropylimidazo[1,2-a]pyridin-2-yl)methyl)pyrimidin-4-amine (23 mg, 0.075 mmol), 6,7-dihydro-5H-cyclopenta[c]pyridine-1,5-diamine (45 mg, 0.30 mmol), Pd-PEPPSI-IPent^{Cl} (o-picoline) (25 mg, 0.030 mmol), BHT (200 mg, 0.91 mmol) and Cs₂CO₃ (300 mg, 0.91 mmol) was placed under a N₂ atmosphere, DMF (18.0 mL) was added and the reaction mixture was stirred at 80° C. for 45 minutes. The reaction mixture was allowed to cool to room temperature, poured into water (75 mL) and extracted with EtOAc (3×30 mL). The combined organic layers were dried over MgSO₄ and concentrated in vacuo. The residue was purified by column chromatography on silica gel, eluting with a gradient of 0-20% NH₃ in MeOH in DCM. The residue was further purified by preparative HPLC to give the title compound (6.7 mg, 21%). ESI-MS (M+H)⁺: 413.3, ¹H NMR (400 MHz, DMSO) δ8.32 (dd, J=0.8, 0.8 Hz, 1H), 7.98 (s, 1H), 7.69 (br s, 1H), 7.61 (s, 1H), 7.37 (d, J=9.2 Hz, 1H), 7.06 (t, J=5.8 Hz, 1H), 6.97 (dd, J=1.7, 9.5 Hz, 1H), 6.92 (d, J=8.4 Hz, 1H), 6.39 (br s, 1H), 5.74 (s, 2H), 5.52 (s, 1H), 5.34 (br s, 1H), 4.46 (d, J=5.3 Hz, 2H), 2.76-2.67 (m, 1H), 2.46-2.37 (m, 1H), 1.96-1.88 (m, 1H), 1.80-1.70 (m, 1H), 0.95-0.89 (m, 2H), 0.70-0.65 (m, 2H). One proton obscured by solvent signal.

### Example 57

### Synthesis of 2-(7-chloroimidazo[1,5-a]pyridin-1-yl)-N-(6-((6-cyclopropylimidazo[1,2-a]pyridin-2-yl)methyl)pyrimidin-4-yl)acetamide formic acid salt (I-57)

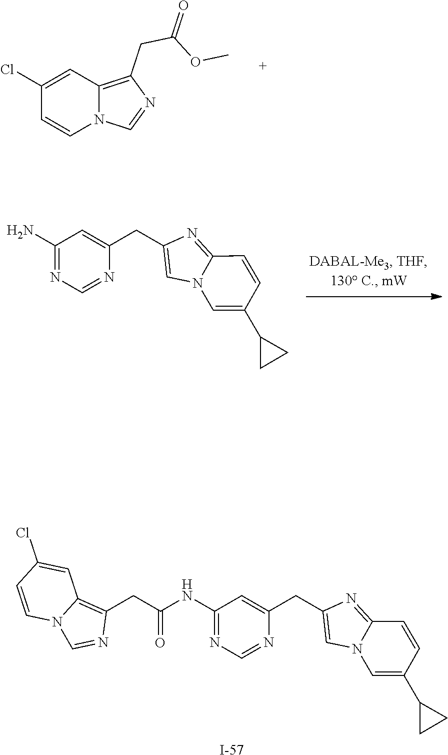

Methyl 2-(7-chloroimidazo[1,5-a]pyridin-1-yl)acetate (120 mg, 0.45 mmol), was added to a microwave vial containing 6-((6-cyclopropylimidazo[1,2-a]pyridin-2-yl)methyl)pyrimidin-4-amine (120 mg, 0.45 mmol), DABAL-Me₃ (120 mg 0.452 mmol) and THF (6 mL). The vial was sealed and heated to 130° C. for 30 min in a CEM microwave. The mixture was diluted with water, extracted with EtOAc and concentrated in vacuo. The residue was purified by reverse phase C₁₈ preparative HPLC gave the title compound (4 mg, 15%). ESI-MS (M+H)⁺: 458.2, ¹H NMR (400 MHz, DMSO) δ11.00 (s, 1H), 8.77 (d, J=1.2 Hz, 1H), 8.50 (s, 0.5H), 8.32-8.27 (m, 3H), 7.93 (d, J=1.1 Hz, 1H), 7.80-7.79 (m, 1H), 7.64 (s, 1H), 7.33 (app-d, J=9.3 Hz, 1H), 6.93 (dd, J=1.7, 9.3 Hz, 1H), 6.65 (dd, J=2.1, 7.5 Hz, 1H), 4.08 (s, 2H), 3.97 (s, 2H), 1.94-1.86 (m, 1H), 0.92-0.86 (m, 2H), 0.68-0.63 (m, 2H).

### Example 58

### Synthesis of 2-(7-chloroimidazo[1,5-a]pyridin-1-yl)-N-(6-(6-cyclopropylimidazo[1,2-a]pyridine-2-carbonyl)pyrimidin-4-yl)acetamide (I-58)

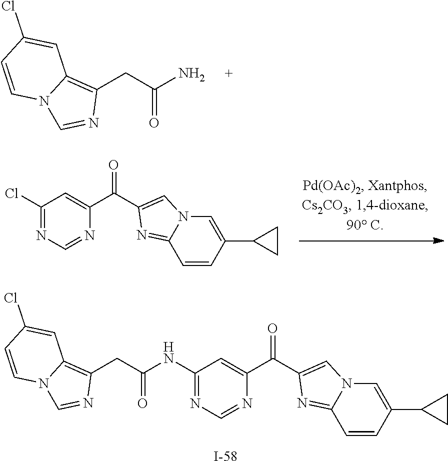

A mixture of (6-chloropyrimidin-4-yl)(6-cyclopropylimidazo[1,2-a]pyridin-2-yl)methanone (100 mg, 0.34 mmol), 2-(7-chloroimidazo[1,5-a]pyridin-1-yl)acetamide (70 mg, 0.34 mmol), Cs₂CO₃ (160 mg, 0.50 mmol), and Xantphos (77 mg, 0.13 mmol) was placed under a N₂ atmosphere, anhydrous 1,4-dioxane (1.0 mL) was added and the suspension was degassed for 10 minutes. Pd(OAc)₂ (15 mg, 0.067 mmol) was added and the reaction mixture was then stirred at 90° C. for 3 hours. The reaction mixture was allowed to cool to room temperature, diluted with water (10 mL), and extracted with DCM (4×10 mL). The combined organics were washed with brine (sat. aq., 10 mL), dried over a hydrophobic frit and concentrated in vacuo. The residue was purified by column chromatography on silica gel, eluting with a gradient of 1-20% 7 N NH₃ in MeOH in DCM. 38 mg of the purified compound was used without further purification and 20 mg of the purified compound was further purified by preparative HPLC to give the title compound (4.3 mg, 3%) as a yellow solid. ESI-MS (M+H)⁺: 472.2, ¹H NMR (400 MHz, DMSO) δ11.41 (s, 1H), 9.10 (d, J=1.1 Hz, 1H), 8.92 (d, J=0.7 Hz, 1H), 8.54-8.52 (m, 1H), 8.47-8.46 (m, 1H), 8.35-8.34 (m, 1H), 8.32 (q, J=2.8 Hz, 1H), 7.85-7.83 (m, 1H), 7.56-7.53 (m, 1H), 7.13 (dd, J=1.8, 9.5 Hz, 1H), 6.66 (dd, J=2.1, 7.5 Hz, 1H), 4.07 (s, 2H), 2.00-1.92 (m, 1H), 0.99-0.93 (m, 2H), 0.76-0.70 (m, 2H).

### Example 59

### Synthesis of 2-(7-chloroimidazo[1,5-a]pyridin-1-yl)-N-(6-((6-cyclopropylimidazo[1,2-a]pyridin-2-yl)(hydroxy)methyl)pyrimidin-4-yl)acetamide (I-59)

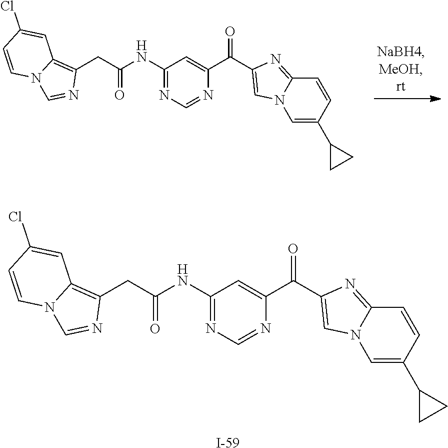

Sodium borohydride (3.1 mg, 0.082 mmol) was added to a cooled solution of 2-(7-chloroimidazo[1,5-a]pyridin-1-yl)-N-(6-(6-cyclopropylimidazo[1,2-a]pyridine-2-carbonyl)pyrimidin-4-yl)acetamide (39 mg, 0.082 mmol) in MeOH (1 mL) at 0° C. and the mixture stirred for 1 h. The mixture was then diluted with water (15 mL), brine (aq. sat. 10 mL) and extracted with EtOAc (3×25 mL). The organic phases were combined and concentrated in vacuo. Purification by reverse phase preparative HPLC gave the title compound (8 mg, 20%) as a white solid. ESI-MS (M+H)⁺: 474.2, ¹H NMR (400 MHz, DMSO) δ ppm 11.04 (s, 1H), 8.73 (d, J=1.2 Hz, 1H), 8.34-8.27 (m, 4H), 7.84-7.82 (m, 1H), 7.66 (s, 1H), 7.35-7.31 (m, 1H), 6.94 (dd, J=1.8, 9.4 Hz, 1H), 6.66 (dd, J=2.1, 7.5 Hz, 1H), 6.10 (d, J=4.9 Hz, 1H), 5.67 (d, J=4.8 Hz, 1H), 4.01 (s, 2H), 1.94-1.86 (m, 1H), 0.93-0.87 (m, 2H), 0.67-0.62 (m, 2H).

### Example 60

### Synthesis of 2-(7-chloroimidazo[1,5-a]pyridin-1-yl)-N-(6-((6-cyclopropylimidazo[1,2-a]pyridin-2-yl)difluoromethyl)pyrimidin-4-yl)acetamide formic acid salt (I-60)

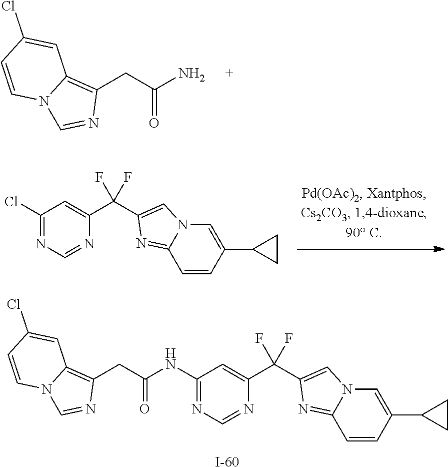

A mixture of 2-((6-chloropyrimidin-4-yl)difluoromethyl)-6-cyclopropylimidazo[1,2-a]pyridine (31 mg, 0.097 mmol), 2-(7-chloroimidazo[1,5-a]pyridin-1-yl)acetamide (20 mg, 0.097 mmol), Cs₂CO₃ (47 mg, 0.15 mmol) and Xantphos (22 mg, 0.039 mmol) was placed under a N₂ atmosphere and stirred for 10 minutes, anhydrous 1,4-dioxane (1.0 mL) was added and the suspension was degassed for 10 minutes. Pd(OAc)₂ (4.3 mg, 0.019 mmol) was added and the reaction mixture was then stirred at 90° C. for 3 hours. The reaction mixture was allowed to cool to room temperature, poured into NaHCO₃ (sat. aq., 10 mL) and extracted with DCM (3×15 mL). The combined organics were washed with brine (sat. aq., 10 mL), dried over a hydrophobic frit and concentrated in vacuo. The residue was purified by column chromatography on silica gel, eluting with 5% MeOH in EtOAc. The residue was further purified by preparative HPLC to give the title compound (1.1 mg, 2%) as a lyophilised white solid. ESI-MS (M+H)⁺: 494.2, ¹H NMR (400 MHz, DMSO) δ11.47 (s, 1H), 8.96 (d, J=1.1 Hz, 1H), 8.54 (s, 1H), 8.43 (d, J=1.3 Hz, 1H), 8.38-8.36 (m, 1H), 8.35-8.34 (m, 1H), 8.32 (q, J=2.8 Hz, 1H), 8.17-8.16 (m, 1H), 7.84-7.83 (m, 1H), 7.47-7.44 (m, 1H), 7.08 (dd, J=1.8, 9.5 Hz, 1H), 6.67 (dd, J=2.1, 7.5 Hz, 1H), 4.06 (s, 2H), 1.99-1.91 (m, 1H), 0.96-0.91 (m, 2H), 0.71-0.66 (m, 2H).

### Example 61

### Synthesis of 2-(7-chloroimidazo[1,5-a]pyridin-1-yl)-N-(6-((6-cyclopropylimidazo[1,2-a]pyridin-2-yl)difluoromethyl)pyrimidin-4-yl)propanamide (I-61)

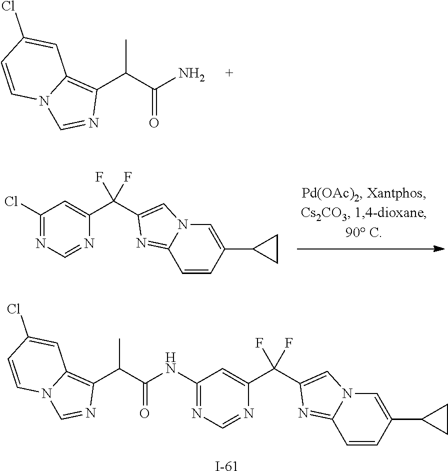

A suspension of 2-((6-chloropyrimidin-4-yl)difluoromethyl)-6-cyclopropylimidazo[1,2-a]pyridine (70 mg, 0.22 mmol), 2-(7-chloroimidazo[1,5-a]pyridin-1-yl)propanamide (54 mg, 0.24 mmol) and Cs₂CO₃ (110 mg, 0.33 mmol) in anhydrous 1,4-dioxane (2.0 mL) was degassed for 10 minutes. Xantphos (51 mg, 0.087 mmol) and Pd(OAc)₂ (9.8 mg, 0.044 mmol) were added, the reaction mixture was placed under a N₂ atmosphere and stirred at 90° C. for 2 hours. The reaction mixture was allowed to cool to room temperature, diluted with EtOAc (10 mL), filtered through Celite® and concentrated in vacuo. The residue was dissolved in 10% MeOH in DCM (10 mL) and water (10 mL), filtered through Celite® and concentrated in vacuo. The residue was purified by preparative HPLC to give the title compound (36 mg, 32%). ESI-MS (M+H)⁺: 508, ¹H NMR (400 MHz, DMSO) δ11.34 (s, 1H), 8.96 (s, 1H), 8.50 (s, 1H), 8.41 (s, 1H), 8.38-8.34 (m, 2H), 8.21 (s, 1H), 7.96 (d, J=1.4 Hz, 1H), 7.50 (d, J=9.4 Hz, 1H), 7.13 (dd, J=1.8, 9.5 Hz, 1H), 6.71 (dd, J=2.1, 7.5 Hz, 1H), 4.42 (q, J=7.0 Hz, 1H), 2.03-1.95 (m, 1H), 1.54 (d, J=7.0 Hz, 3H), 1.01-0.95 (m, 2H), 0.75-0.70 (m, 2H).

### Example 62

### Synthesis of 6-((6-cyclopropylimidazo[1,2-a]pyridin-2-yl)difluoromethyl)-N-(2-fluoro-3-methoxy-6-(1H-tetrazol-1-yl)benzyl)pyrimidin-4-amine (I-62)

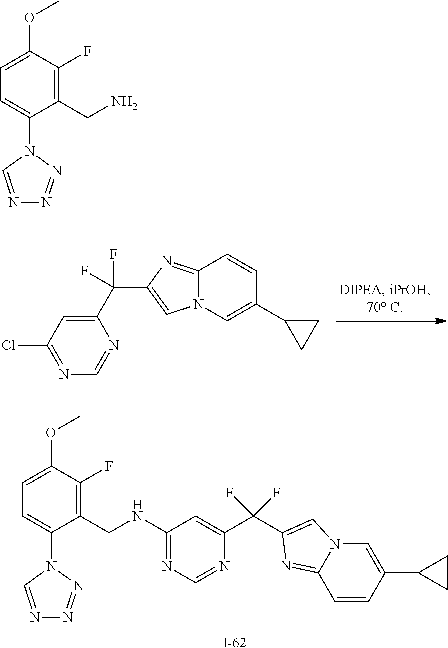

DIPEA (0.041 mL, 0.23 mmol) was added to a mixture of (2-fluoro-3-methoxy-6-(1H-tetrazol-1-yl)phenyl)methanamine (21 mg, 0.093 mmol, see WO2017207983) and 2-((6-chloropyrimidin-4-yl)difluoromethyl)-6-cyclopropylimidazo[1,2-a]pyridine (30 mg, 0.093 mmol) in iPrOH and heated to 70° C. for 18 h. The mixture was concentrated in vacuo and purification by reverse phase preparative HPLC gave the title compound. ESI-MS (M+H)⁺: 508.2, ¹H NMR (400 MHz, DMSO) δ9.82 (s, 1H), 8.40 (s, 1H), 8.23 (s, 2H), 8.12 (s, 1H), 7.50-7.45 (m, 2H), 7.41 (dd, J=8.8, 8.8 Hz, 1H), 7.11 (dd, J=1.8, 9.5 Hz, 1H), 6.82 (s, 1H), 4.43 (d, J=4.9 Hz, 2H), 3.98 (s, 3H), 2.03-1.95 (m, 1H), 1.00-0.95 (m, 2H), 0.74-0.70 (m, 2H).

### Example 63

### Synthesis of (6-cyclopropylimidazo[1,2-a]pyridin-2-yl)(6-((2-fluoro-3-methoxy-6-(H-tetrazol-1-yl)benzyl)amino)pyrimidin-4-yl)methanol (I-63)

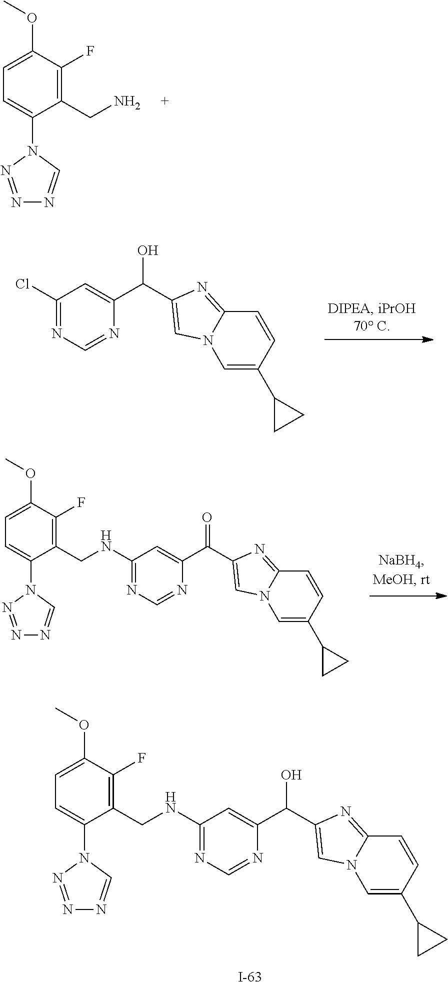

Synthesis of (6-cyclopropylimidazo[1,2-a]pyridin-2-yl)(6-((2-fluoro-3-methoxy-6-(1H-tetrazol-1-yl)benzyl)amino)pyrimidin-4-yl)methanone. (6-Chloropyrimidin-4-yl)(6-cyclopropylimidazo[1,2-a]pyridin-2-yl)methanol (50 mg, 0.166 mmol) was added to a mixture of (2-fluoro-3-methoxy-6-(1H-tetrazol-1-yl)phenyl)methanamine (37 mg, 0.166 mmol, see WO2017207983) and DIPEA 0.072 mL, 0.416 mmol) in iPrOH (2 mL) and the mixture was heated to 70° C. for 18 h. The mixture was concentrated in vacuo. Purification by column chromatography on silica gel, eluting with a gradient of 0-20% MeOH in DCM gave the title compound (50 mg, 61%) which was used in the next step without further purification.

Synthesis of (6-cyclopropylimidazo[1,2-a]pyridin-2-yl)(6-((2-fluoro-3-methoxy-6-(1H-tetrazol-1-yl)benzyl)amino)pyrimidin-4-yl)methanol. Sodium borohydride (4.3 mg, 0.133 mmol) was added to a cooled solution of (6-cyclopropylimidazo[1,2-a]pyridin-2-yl)(6-((2-fluoro-3-methoxy-6-(1H-tetrazol-1-yl)benzyl)amino)pyrimidin-4-yl)methanone (50 mg, 0.103 mmol) in MeOH (2 mL) at 0° C. The mixture was allowed to warm to room temperature and stirred for 1 h. The mixture was diluted with water (15 mL), brine (aq. sat. 10 mL) and extracted with DCM (3×25 mL). The organic phases were combined, passed through a phase separator cartridge and concentrated in vacuo. Purification by reverse phase preparative HPLC gave the title compound (13 mg, 19%) as a white solid. ESI-MS (M+H)⁺: 488.3, ¹H NMR (400 MHz, DMSO) δ9.78 (s, 1H), 8.29 (s, 1H), 8.07 (s, 1H), 7.79 (br s, 1H), 7.60 (s, 1H), 7.42 (dd, J=0.9, 8.9 Hz, 1H), 7.38-7.32 (m, 2H), 6.95 (dd, J=1.7, 9.3 Hz, 1H), 6.64 (s, 1H), 5.81 (d, J=5.1 Hz, 1H), 5.44 (d, J=4.2 Hz, 1H), 4.34 (d, J=5.1 Hz, 2H), 3.95 (s, 3H), 1.95-1.88 (m, 1H), 0.92 (ddd, J=4.2, 6.2, 8.3 Hz, 2H), 0.69-0.64 (m, 2H).

### Example 64

### Synthesis of (6-(((6-amino-2,4-dimethylpyridin-3-yl)methyl)amino)pyrimidin-4-yl)(6-cyclopropylimidazo[1,2-a]pyridin-2-yl)methanol bisformic acid salt (I-64)

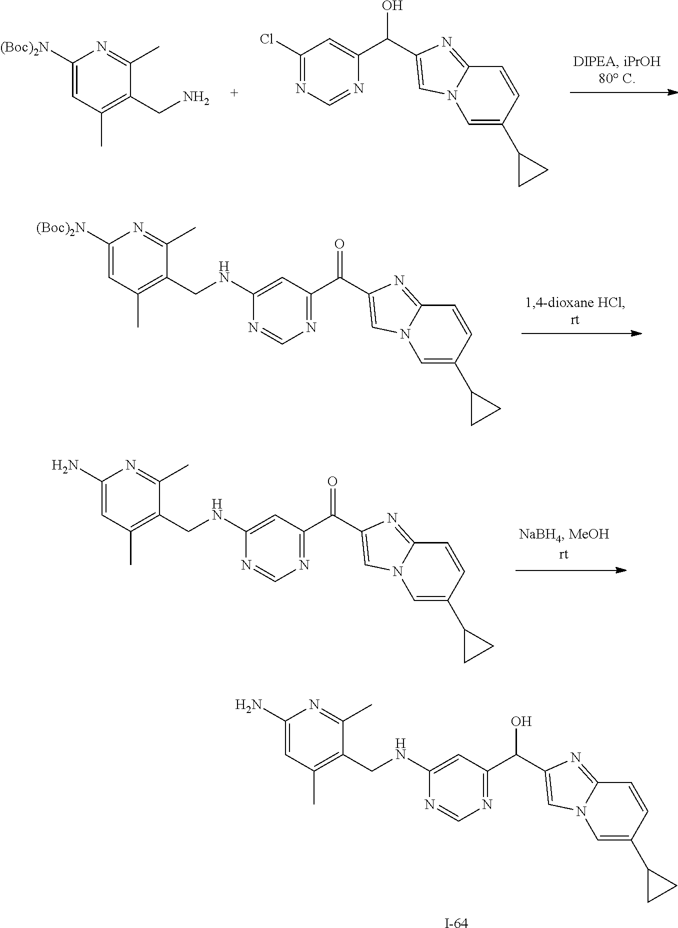

Synthesis of tert-butyl-(tert-butoxycarbonyl)(5-(((6-(6-cyclopropylimidazo[1,2-a]pyridine-2-carbonyl)pyrimidin-4-yl)amino)methyl)-4,6-dimethylpyridin-2-yl)carbamate. Tert-butyl-(5-(aminomethyl)-4,6-dimethylpyridin-2-yl)(tert-butoxycarbonyl)carbamate (129 mg, 0.22 mmol, see WO2016201052) was added to a mixture of DIPEA (0.087 mL, 0.50 mmol) and (6-chloropyrimidin-4-yl)(6-cyclopropylimidazo[1,2-a]pyridin-2-yl)methanol (60 mg, 0.20 mmol) in iPrOH (2 mL). The mixture was heated to 80° C. for 6 h. The mixture was concentrated in vacuo to give the crude title compound and which was used in the next step without further purification.

Synthesis of (6-(((6-amino-2,4-dimethylpyridin-3-yl)methyl)amino)pyrimidin-4-yl)(6-cyclopropylimidazo[1,2-a]pyridin-2-yl)methanone. Crude tert-butyl-(tert-butoxycarbonyl)(5-(((6-(6-cyclopropylimidazo[1,2-a]pyridine-2-carbonyl)pyrimidin-4-yl)amino)methyl)-4,6-dimethylpyridin-2-yl)carbamate in 1,4-dioxane (1 mL) was treated with HCl in 1,4-doixane (4M, 1 mL, 4.0 mmol) and stirred at room temperature for 1 h. The mixture was concentrated in vacuo to give the crude title compound which was used in the next step without further purification.

Synthesis of (6-(((6-amino-2,4-dimethylpyridin-3-yl)methyl)amino)pyrimidin-4-yl)(6-cyclopropylimidazo[1,2-a]pyridin-2-yl)methanol bis formic acid salt. Sodium borohydride (7 mg, 0.17 mmol) was added to a cooled solution of (6-(((6-amino-2,4-dimethylpyridin-3-yl)methyl)amino)pyrimidin-4-yl)(6-cyclopropylimidazo[1,2-a]pyridin-2-yl)methanone (70 mg, 0.17 mmol) in MeOH (5 mL) at 0° C. The reaction was allowed to warm to room temperature and was then stirred for 1 h. The mixture was diluted with water (15 mL) and brine (aq. sat. 10 mL) and extracted with EtOAc (3×25 mL). The organic phases were combined and concentrated in vacuo. Purification by reverse phase preparative HPLC gave the title compound (13 mg, 19%) as a white solid. ESI-MS (M+H)⁺: 416.3, ¹H NMR (400 MHz, DMSO) δ8.36 (s, 1H), 8.34 (s, 1H), 8.20 (s, 1H), 7.66 (s, 1H), 7.38 (d, J=9.3 Hz, 1H), 7.31 (s, 1H), 6.98 (dd, J=1.8, 9.3 Hz, 1H), 6.73 (s, 1H), 6.16 (s, 1H), 5.81-5.77 (m, 1H), 5.72 (s, 2H), 5.50 (s, 1H), 4.36 (d, J=3.1 Hz, 2H), 2.32 (s, 3H), 2.19 (s, 3H), 1.99-1.90 (m, 1H), 0.95 (ddd, J=4.1, 6.1, 8.4 Hz, 2H), 0.72-0.67 (m, 2H).

### Example 65

### Synthesis of N-((7-chloroimidazo[1,5-a]pyridin-1-yl)methyl)-6-(((6-cyclopropylimidazo[1,2-a]pyridin-2-yl)methyl)amino)pyridazine-4-carboxamide (I-65)

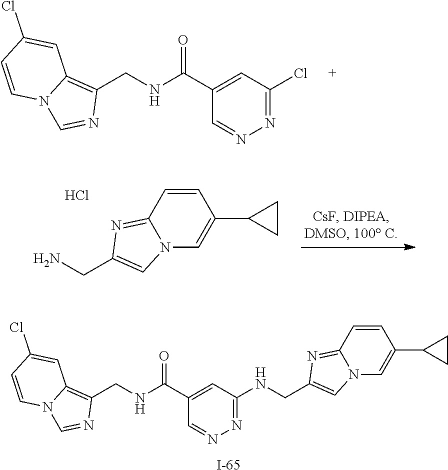

Cesium fluoride (94 mg, 0.621 mmol) was added to a mixture of 6-chloro-N-((7-chloroimidazo[1,5-a]pyridin-1-yl)methyl)pyridazine-4-carboxamide (100 mg, 0.310 mmol), (6-cyclopropylimidazo[1,2-a]pyridin-2-yl)methanamine hydrochloride (104 mg, 0.466 mmol) and DIPEA (0.16 mL, 0.931 mmol) in DMSO (2.2 mL) and the mixture was heated to 110° C. for 18 h. The mixture was diluted with water, filtered and the solid was washed with water. The solid was purified by column chromatography on silica gel, eluting with a gradient of 0-10% MeOH in DCM to give the title compound (5.6 mg, 4%) as an off white solid. ESI-MS (M+H)⁺:473.3, ¹H NMR (400 MHz, DMSO) δ 9.29 (dd, J=5.5, 5.5 Hz, 1H), 8.77 (d, J=1.8 Hz, 1H), 8.36-8.33 (m, 2H), 8.30 (s, 1H), 7.83 (d, J=1.4 Hz, 1H), 7.66 (s, 1H), 7.55 (dd, J=5.6, 5.6 Hz, 1H), 7.39 (d, J=9.3 Hz, 1H), 7.22 (d, J=1.8 Hz, 1H), 6.97 (dd, J=1.8, 9.3 Hz, 1H), 6.69 (dd, J=2.1, 7.5 Hz, 1H), 4.69-4.63 (m, 4H), 1.96-1.88 (m, 1H), 0.95-0.89 (m, 2H), 0.70-0.64 (m, 2H).

### Example 66

### Synthesis of N-((7-chloroimidazo[1,5-a]pyridin-1-yl)methyl)-6-((6-cyclopropylimidazo[1,2-a]pyridin-2-yl)methoxy)pyridazine-4-carboxamide (I-66)

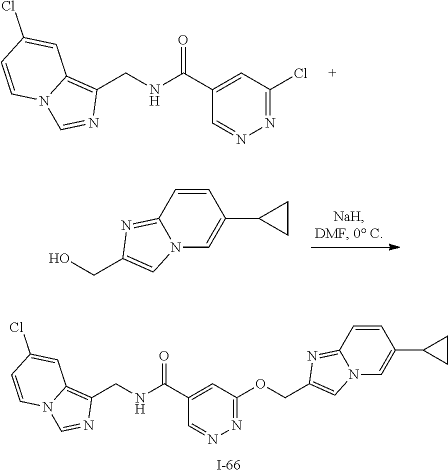

Sodium hydride (60% in mineral oil, 1.5 mg, 0.038 mmol) was added to a cooled solution of (6-cyclopropylimidazo[1,2-a]pyridin-2-yl)methanol (7 mg, 0.038 mmol) in DMF (0.4 mL) at 0° C. After stirring for 20 min 6-chloro-N-((7-chloroimidazo[1,5-a]pyridin-1-yl)methyl)pyridazine-4-carboxamide (11 mg, 0.034 mmol) was added, the mixture was allowed to warm to room temperature and stirred for 18 h. The mixture was quenched with the addition of iPrOH and concentrated in vacuo. The residue was dissolved in DCM, washed with water, passed through a hydrophobic cartridge and concentrated in vacuo. Purification by column chromatography on silica gel, eluting with a gradient of 0-5% MeOH in DCM gave the title compound (1.1 mg, 6%) as an off white solid. ESI-MS (M+H)⁺: 474.2, ¹H NMR (400 MHz, DMSO) δ9.44 (t, J=5.6 Hz, 1H), 9.24-9.22 (m, 1H), 8.37-8.33 (m, 3H), 7.93-7.89 (m, 1H), 7.86-7.84 (m, 1H), 7.56-7.54 (m, 1H), 7.46-7.42 (m, 1H), 7.05-7.00 (m, 1H), 6.71-6.67 (m, 1H), 5.65-5.62 (m, 2H), 4.70-4.67 (m, 2H), 1.98-1.91 (m, 1H), 0.97-0.91 (m, 2H), 0.72-0.67 (m, 2H).

### Example 67

### Synthesis of 2-(3-chlorophenyl)-N-(6-(((6-cyclopropylimidazo[1,2-a]pyridin-2-yl)methyl)amino)pyrimidin-4-yl)cyclopropane-1-sulfonamide (I-67)

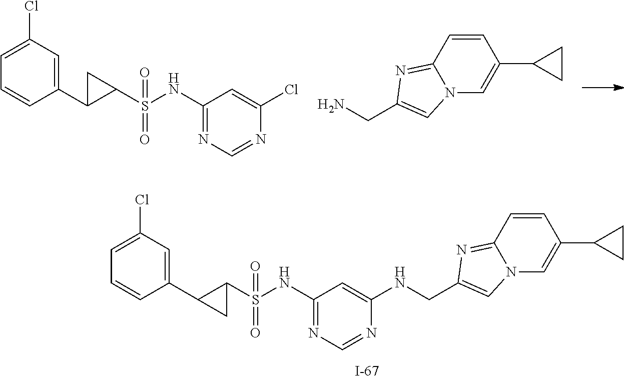

The title compound (49 mg, 44%) was prepared using a similar procedure to that that used for the preparation of ethyl 3-(2-(((6-(2-(7-chloroimidazo[1,5-a]pyridin-1-yl)acetamido)pyrimidin-4-yl)amino)methyl)-6-cyclopropylimidazo[1,2-a]pyridin-8-yl)propanoate (Example 38) by using 2-(3-chlorophenyl)-N-(6-chloropyrimidin-4-yl)cyclopropane-1-sulfonamide and (6-cyclopropylimidazo[1,2-a]pyridin-2-yl)methanamine as coupling partners and purified by preparative HPLC. ESI-MS (M+H)⁺: 495.2, ¹H NMR (400 MHz, DMSO) δ8.32-8.32 (m, 1H), 8.12 (s, 1H), 8.04 (s, 1H), 7.66 (s, 1H), 7.41-7.37 (m, 1H), 7.32-7.23 (m, 3H), 7.13-7.09 (m, 1H), 6.99 (dd, J=1.9, 9.2 Hz, 1H), 6.16 (s, 1H), 4.58-4.53 (m, 2H), 2.97-2.91 (m, 1H), 1.98-1.90 (m, 1H), 1.53-1.47 (m, 1H), 1.38-1.34 (m, 1H), 0.97-0.91 (m, 2H), 0.71-0.66 (m, 2H). 2 protons not observed, one obscured by residual solvent and NH sulphonamide signal also not observed.

### Example 68

### Synthesis of 3-(((7-chloroimidazo[1,5-a]pyridin-1-yl)methyl)amino)-4-(((6-cyclopropylimidazo[1,2-a]pyridin-2-yl)methyl)amino)cyclobut-3-ene-1,2-dione (I-68)

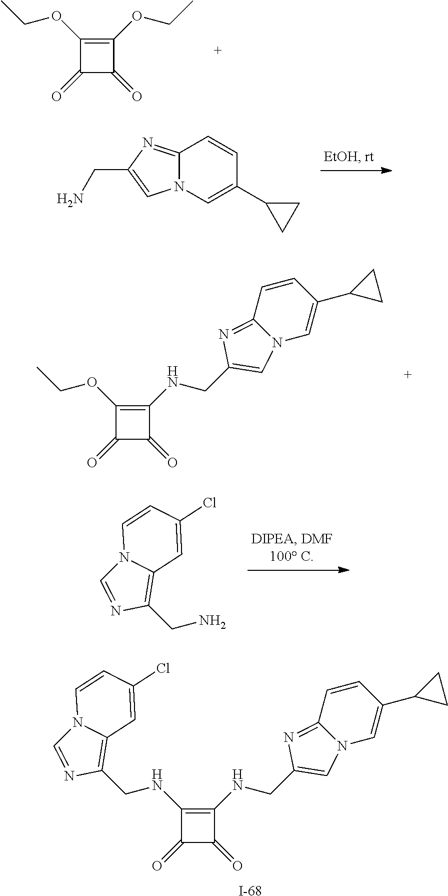

A suspension of 3,4-diethoxy-3-cyclobutene-1,2-dione (0.062 g, 0.36 mmol) and (6-cyclopropylimidazo[1,2-a]pyridin-2-yl)methanamine (0.075 g, 0.40 mmol) in EtOH (2 mL) was stirred at room temperature for 4 h. The mixture was concentrated in vacuo and purified by silica gel column chromatography using a 0-5% MeOH in DCM gradient to afford 3-(((6-cyclopropylimidazo[1,2-a]pyridin-2-yl)methyl)amino)-4-ethoxycyclobut-3-ene-1,2-dione (0.066 g, 58%) which was used directly without further purification.

A suspension of 3-(((6-cyclopropylimidazo[1,2-a]pyridin-2-yl)methyl)amino)-4-ethoxycyclobut-3-ene-1,2-dione (0.066 g, 0.21 mmol) and (7-chloroimidazo[1,5-a]pyridin-1-yl)methanamine (0.042 g, 0.23 mmol, see WO2019178129) in EtOH (2 mL) was stirred at room temperature for 72 h. The reaction was then heated to 50° C. for 4 h. DMF (1 mL) was then added and the reaction stirred at room temperature for 2.5 h. The EtOH was then removed by evaporation and replaced with DMF (2 mL). DIPEA (53 μL, 0.21 mmol) was added and the reaction stirred at 100° C. for 24 hours. The reaction was cooled to room temperature and the solvent removed in vacuo. The residue was purified by preparative HPLC to give the title compound (0.007 g, 17%). ESI-MS (M+H)+: 447.2, ¹H NMR (400 z, DMSO) δ8.45-8.36 (M, 3H), 7.88-7.84 (m, 2H), 7.78-7.75 (m, 1H), 7.47-7.41 (i, 1H), 7.06-7.03 (m, 1H), 6.76-6.72 (m, 1H), 5.01-4.94 (i, 2H), 4.86-4.79 (i, 2H), 2.01-1.93 (m, 1H), 1.00-0.94 (m, 2H), 0.75-0.69 (m, 2H). One exchangeable proton was not observed.

| EG | Compound | Analytical Data |
|---|---|---|
| 69 | 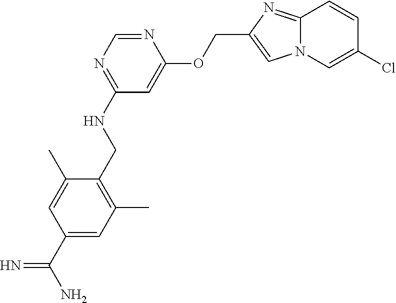 [SMILES: Cc1cc(C(=N)N)cc(C)c1CNc1cc(OCc2cn3cc(Cl)ccc3n2)ncn1] | ESI-MS [M + H]⁺: 436.1. ¹H NMR (400 MHz, DMSO) δ 9.05 (s, 1H), 8.76 (d, J = 1.3 Hz, 1H), 8.39 (s, 1H), 8.21 (s, 1H), 7.88 (s, 1H), 7.52 (d, J = 9.6 Hz, 1H), 7.46 (s, 2H), 7.33-7.03 (m, 2H), 5.82 (s, 1H), 5.34 (s, 2H), 4.47 (s, 2H), 2.37 (s, 6H). |
| | 4-(((6-((6-chloroimidazo[1,2-a]pyridin-2- | |
| | yl)methoxy)pyrimidin-4-yl)amino)methyl)-3,5- | |
| | dimethylbenzimidamide formic acid salt (I-69) | |
| 70 | 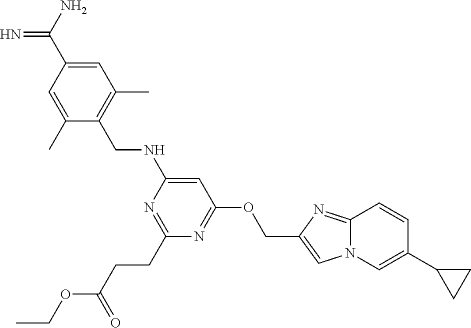 [SMILES: CCOC(=O)CCc1nc(NCc2c(C)cc(C(=N)N)cc2C)cc(OCc2cn3cc(C4CC4)ccc3n2)n1] | ESI-MS [M + H]⁺: 542.2. ¹H NMR (400 MHz, DMSO) δ 9.31-9.27 (m, 2H), 9.11-8.98 (m, 2H), 8.75-8.70 (m, 1H), 8.26-8.22 (m, 1H), 7.84-7.82 (m, 1H), 7.71-7.69 (m, 1H), 7.56-7.39 (m, 3H), 5.77 (s, 1H), 5.52 (s, 2H), 4.54 (s, 2H), 4.05-4.00 (m, 2H), 2.95 (s, 2H), 2.78- 2.72 (m, 2H), 2.42 (s, 6H), 2.12-2.01 (m, 1H), 1.13-1.11 (m, 3H), 1.07-1.03 (m, 2H), 0.81-0.79 (m, 2H). |
| | ethyl 3-(4-((4-carbamimidoyl-2,6- | |
| | dimethylbenzyl)amino)-6-((6-cyclo propyl | |
| | imidazo[1,2-a]pyridin-2-yl)methoxy)pyrimidin- | |
| | 2-yl)propanoate formic acid salt | |
| | (I-70) | |
| 71 | 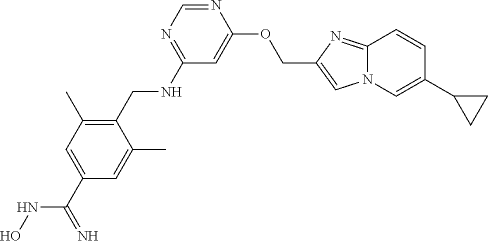 [SMILES: Cc1cc(C(=N)NO)cc(C)c1CNc1cc(OCc2cn3cc(C4CC4)ccc3n2)ncn1] | ESI-MS [M + H]⁺: 458.2. ¹H NMR (400 MHz, DMSO) δ 9.53 (s, 1H), 8.33 (s, 1H), 8.25 (s, 1H), 7.81 (s, 1H), 7.40 (d, J = 9.3 Hz, 1H), 7.36 (s, 2H), 7.15 (t, J = 4.5 Hz, 1H), 6.99 (m, 1H), 5.85 (s, 1H), 5.72 (s, 2H), 5.34 (s, 2H), 4.44 (s, 2H), 2.33 (s, 6H), 1.94-1.88 (m, 1H), 0.95-0.89 (m, 2H), 0.70-0.65 (m, 2H). |
| | 4-(((6-((6-cyclopropylimidazo[1,2-a]pyridin-2- | |
| | yl)methoxy)pyrimidin-4-yl)amino)methyl)-N- | |
| | hydroxy-3,5-dimethylbenzimidamide (I-71) | |
| 72 | 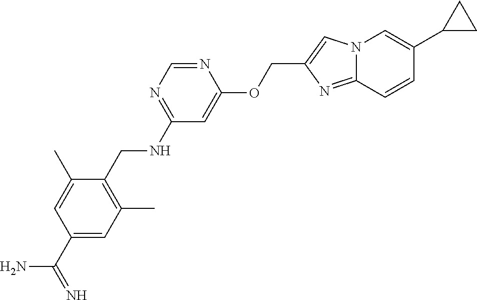 [SMILES: Cc1cc(C(=N)N)cc(C)c1CNc1cc(OCc2cn3cc(C4CC4)ccc3n2)ncn1] | ESI-MS [M + H]⁺: 442.2. ¹H NMR (400 MHz, DMSO) δ 11.16 (s, 2H), 8.92 (s, 1H), 8.42 (s, 1H), 8.33 (s, 1H), 8.26 (s, 1H), 7.81 (s, 1H), 7.50 (s, 2H), 7.40 (d, J = 9.3 Hz, 1H), 7.30 (t, J = 4.7 Hz, 1H), 6.99 (dd, J = 9.3, 1.6 Hz, 1H), 5.86 (s, 1H), 5.35 (s, 2H), 4.52 (s, 2H), 2.42 (s, 6H), 2.07-1.62 (m, 1H), 1.05- 0.83 (m, 2H), 0.79-0.42 (m, 2H). |
| | 4-(((6-((6-cyclopropylimidazo[1,2-a]pyridin-2- | |
| | yl)methoxy)pyrimidin-4-yl)amino)methyl)-3,5- | |
| | dimethylbenzimidamide formic acid salt (I-72) | |
| 73 | 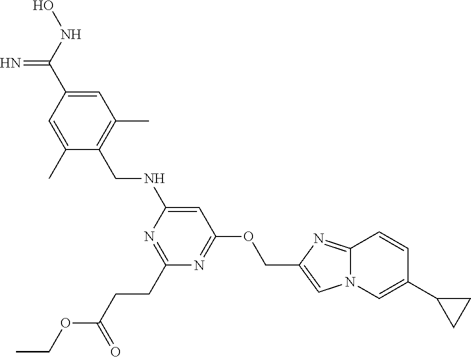 [SMILES: CCOC(=O)CCc1nc(NCc2c(C)cc(C(=N)NO)cc2C)cc(OCc2cn3cc(C4CC4)ccc3n2)n1] | ESI-MS [M + H]⁺: 558.2. ¹H NMR (400 MHz, DMSO) δ 9.52 (s, 1H), 8.33 (s, 1H), 7.80 (s, 1H), 7.40 (d, J = 9.3 Hz, 1H), 7.35 (s, 2H), 7.06-7.02 (m, 1H), 7.01-6.98 (m, 1H), 5.71 (s, 2H), 5.67 (s, 1H), 5.32 (s, 2H), 4.43 (s, 2H), 4.06- 4.01 (m, 2H), 2.95-2.92 (m, 2H), 2.76- 2.71 (m, 2H), 2.32 (s, 6H), 1.96-1.89 (m, 1H), 1.13 (t, J = 7.1 Hz, 3H), 0.94- 0.90 (m, 2H), 0.71-0.63 (m, 2H). |
| | ethyl 3-(4-((6-cyclopropylimidazo[1,2-a]pyridin- | |
| | 2-yl)methoxy)-6-((4-(N-hydroxy | |
| | carbamimidoyl)-2,6-dimethylbenzyl) | |
| | amino)pyrimidin-2-yl)propanoate (I-73) | |
| 74 | 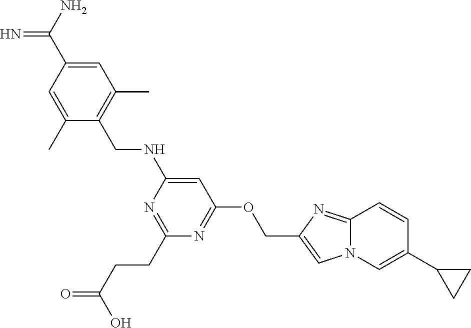 [SMILES: Cc1cc(C(=N)N)cc(C)c1CNc1cc(OCc2cn3cc(C4CC4)ccc3n2)nc(CCC(=O)O)n1] | ESI-MS [M + H]⁺: 514.2. ¹H NMR (400 MHz, DMSO) δ 11.31-11.20 (m, 2H), 9.08-8.73 (m, 1H), 8.44 (s, 1H), 8.32 (s, 1H), 7.82 (s, 1H), 7.44-6.98 (m, 3H), 7.16 (s, 1H), 6.99 (d, J = 9.4 Hz, 1H), 5.64 (s, 1H), 5.33 (s, 2H), 4.54 (s, 2H), 2.86-2.82 (m, 2H), 2.67-2.62 (m, 2H), 2.41 (s, 6H), 1.93-1.91 (m, 1H), 0.93- 0.91 (m, 2H), 0.69-0.67 (m, 2H). |
| | 3-(4-((4-carbamimidoyl-2,6-dimethylbenzyl) | |
| | amino)-6-((6-cyclopropylimidazo[1,2-a]pyridin- | |
| | 2-yl)methoxy)pyrimidin-2-yl)propanoic acid | |
| | formic acid salt (I-74) | |
| 75 | 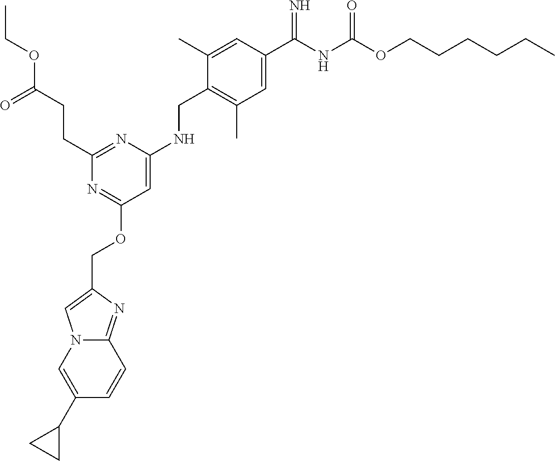 [SMILES: CCCCCCOC(=O)NC(=N)c1cc(C)c(CNc2cc(OCc3cn4cc(C5CC5)ccc4n3)nc(CCC(=O)OCC)n2)c(C)c1] | ESI-MS [M + H]⁺: 670.3. ¹H NMR (400 MHz, DMSO) δ 9.13-8.94 (m, 2H), 8.33 (s, 1H), 7.80 (s, 1H), 7.66 (s, 2H), 7.40 (d, J = 9.3 Hz, 1H), 7.12 (t, J = 4.6 Hz, 1H), 7.00-6.97 (m, 1H), 5.67 (s, 1H), 5.32 (s, 2H), 4.48 (s, 2H), 4.11- 3.94 (m, 4H), 2.95-2.92 (m, 2H), 2.75- 2.72 (m, 2H), 2.38 (s, 6H), 1.95-1.89 (m, 1H), 1.65-1.53 (m, 2H), 1.41- 1.27 (m, 6H), 1.13 (t, J = 7.1 Hz, 3H), 0.97-0.82 (m, 5H), 0.73-0.62 (m, 2H) |
| | ethyl 3-(4-((6-cyclopropylimidazo[1,2-a]pyridin- | |
| | 2-yl)methoxy)-6-((4-(N-((hexyloxy) | |
| | carbonyl)carbamimidoyl)-2,6-dimethylbenzyl) | |
| | amino)pyrimidin-2-yl)propanoate (I-75) | |
| 76 | 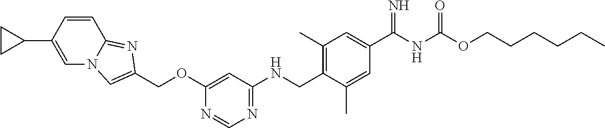 [SMILES: CCCCCCOC(=O)NC(=N)c1cc(C)c(CNc2cc(OCc3cn4cc(C5CC5)ccc4n3)ncn2)c(C)c1] | ESI-MS [M + H]⁺: 570.3. ¹H NMR (400 MHz, DMSO) δ 9.15 (s, 1H), 8.94 (s, 1H), 8.33 (s, 1H), 8.26 (s, 1H), 7.81 (s, 1H), 7.67 (s, 2H), 7.40 (d, J = 9.3 Hz, 1H), 7.22 (t, J = 4.6 Hz, 1H), 7.01-6.97 (m, 1H), 5.86 (s, 1H), 5.35 (s, 2H), 4.48 (s, 2H), 4.00 (t, J = 6.7 Hz, 2H), 2.38 (s, 6H), 1.97-1.88 (m, 1H), 1.62-1.55 (m, 2H), 1.41-1.17 (m, 6H), 1.00-0.78 (m, 5H), 0.74-0.60 (m, 2H). |
| | hexyl ((4-(((6-((6-cyclopropylimidazo[1,2- | |
| | a]pyridin-2-yl)methoxy)pyrimidin-4-yl) | |
| | amino)methyl)-3,5-dimethylphenyl) | |
| | (imino)methyl)carbamate (I-76) | |
| 77 | 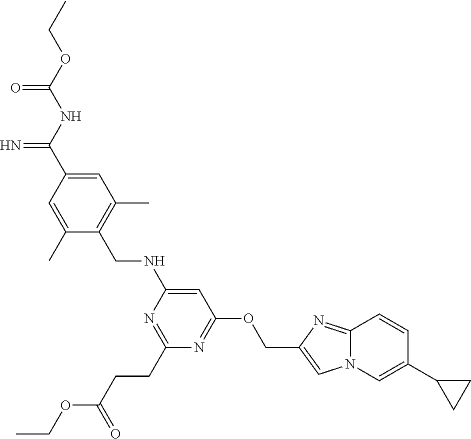 [SMILES: CCOC(=O)CCc1nc(NCc2c(C)cc(C(=N)NC(=O)OCC)cc2C)cc(OCc2cn3cc(C4CC4)ccc3n2)n1] | ESI-MS [M + H]⁺: 614.3. ¹H NMR (400 MHz, DMSO) δ 9.14-8.95 (m, 2H), 8.33(s, 1H), 7.80 (s, 1H), 7.67 (s, 2H), 7.40 (d, J = 9.3 Hz, 1H), 7.13-7.11 (m, 1H), 7.01-6.97 (m, 1H), 5.67 (s, 1H), 5.32 (s, 2H), 4.48 (s, 2H), 4.09-3.99 (m, 4H), 2.94-2.91 (m, 2H), 2.76-2.67 (m, 2H), 2.38 (s, 6H), 1.96-1.89 (m, 1H), 1.33-1.29 (m, 3H), 1.15-1.11 (m, 3H), 0.95-0.86 (m, 2H), 0.71-0.62 (m, 2H). |
| | ethyl 3-(4-((6-cyclopropylimidazo[1,2-a]pyridin- | |
| | 2-yl)methoxy)-6-((4-(N- | |
| | (ethoxycarbonyl)carbamimidoyl)-2,6-dimethyl | |
| | benzyl)amino)pyrimidin-2-yl)propanoate (I-77) | |
| 78 | 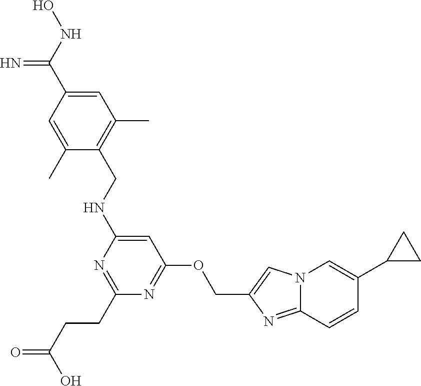 [SMILES: Cc1cc(C(=N)NO)cc(C)c1CNc1cc(OCc2cn3cc(C4CC4)ccc3n2)nc(CCC(=O)O)n1] | ESI-MS [M + H]⁺: 530.2. ¹H NMR (400 MHz, DMSO) δ 9.52 (s, 1H), 8.32 (s, 1H), 8.20 (s, 1H), 7.81 (s, 1H), 7.40 (d, J = 9.3 Hz, 1H), 7.35 (s, 2H), 7.06- 6.95 (m, 2H), 5.71 (s, 2H), 5.66 (s, 1H), 5.32 (s, 2H), 4.44 (s, 2H), 2.90-2.87 (m, 2H), 2.71-2.64 (m, 2H), 2.33 (s, 6H), 1.96-1.90 (m, 1H), 0.95-0.88 (m, 2H), 0.71-0.65 (m, 2H). |
| | 3-(4-((6-cyclopropylimidazo[1,2-a]pyridin-2- | |
| | yl)methoxy)-6-((4-(N-hydroxycarbamimidoyl)- | |
| | 2,6-dimethylbenzyl)amino)pyrimidin-2-yl) | |
| | propanoic acid formic acid salt (I-78) | |
| 79 | 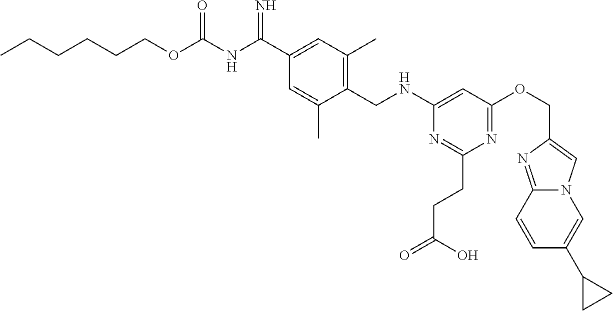 [SMILES: CCCCCCOC(=O)NC(=N)c1cc(C)c(CNc2cc(OCc3cn4cc(C5CC5)ccc4n3)nc(CCC(=O)O)n2)c(C)c1] | ESI-MS [M + H]⁺: 642.4. ¹H NMR (400 MHz, DMSO) δ 9.16-9.12(m, 2H), 8.54- 8.25 (m, 2H), 7.89-7.78 (m, 1H), 7.73-7.60 (m, 2H), 7.47-7.32 (m, 1H), 7.20-7.07 (m, 1H), 7.07-6.88 (m, 1H), 5.73-5.57 (m, 1H), 5..33 (s, 2H), 4.49 (s, 2H), 4.11-3.92 (m, 2H), 2.96-2.80 (m, 2H), 2.74-2.63 (m, 2H), 2.38 (s, 6H), 1.99-1.89 (m, 1H), 1.66-1.51 (m, 2H), 1.39-1.21 (m, 6H), 0.98-0.81 (m, 5H), 0.77-0.57 (m, 2H). |
| | 3-(4-((6-cyclopropylimidazo[1,2-a]pyridin-2- | |
| | yl)methoxy)-6-((4-(N-((hexyloxy)carbonyl) | |
| | carbamimidoyl)-2,6-dimethylbenzyl) | |
| | amino)pyrimidin-2-yl)propanoic acid (I-79) | |
| 80 | 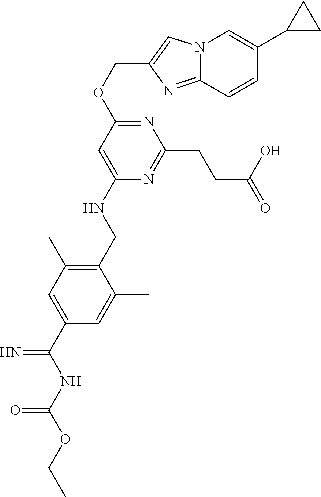 [SMILES: CCOC(=O)NC(=N)c1cc(C)c(CNc2cc(OCc3cn4cc(C5CC5)ccc4n3)nc(CCC(=O)O)n2)c(C)c1] | ESI-MS [M + H]⁺: 586.3. ¹H NMR (400 MHz, DMSO) δ 9.15-8.92 (m, 2H), 8.32 (s, 1H), 7.81 (s, 1H), 7.66 (s, 2H), 7.40 (d, J = 9.3 Hz, 1H), 7.13-7.09 (m, 1H), 6.99 (d, J = 9.3 Hz, 1H), 5.67 (s, 1H), 5.33 (s, 2H), 4.49 (s, 2H), 4.08- 4.02 (m, 2H), 2.89 (t, J = 6.5 Hz, 2H), 2.68 (t, J = 6.6 Hz, 2H), 2.38 (s, 6H), 1.97-1.88 (m, 1H), 1.22 (t, J = 7.1 Hz, 3H), 0.94-0.90 (m, 2H), 0.70-0.58 (m, 2H). |
| | 3-(4-((6-cyclopropylimidazo[1,2-a]pyridin-2- | |
| | yl)methoxy)-6-((4-(N-(ethoxycarbonyl) | |
| | carbamimidoyl)-2,6-dimethylbenzyl) | |
| | amino)pyrimidin-2-yl)propanoic acid (I-80) | |
| 81 | 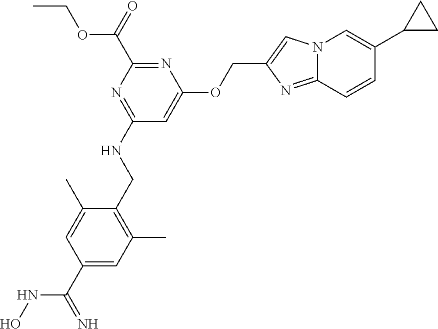 [SMILES: CCOC(=O)c1nc(NCc2c(C)cc(C(=N)NO)cc2C)cc(OCc2cn3cc(C4CC4)ccc3n2)n1] | ESI-MS [M + H]⁺: 530.3. ¹H NMR (400 MHz, DMSO) δ 9.52 (s, 1H), 8.33 (s, 1H), 7.85 (s, 1H), 7.55 (s, 1H), 7.41 (d, J = 9.3 Hz, 1H), 7.34 (s, 2H), 7.00 (dd, J = 9.4, 1.7 Hz, 1H), 5.99 (s, 1H), 5.71 (s, 2H), 5.37 (s, 2H), 4.47 (s, 2H), 4.32 (q, J = 7.1 Hz, 2H), 2.33 (s, 6H), 1.93- 1.90 (m, 1H), 1.32 (t, J = 7.1 Hz, 3H), 1.03-0.80 (m, 2H), 0.80-0.54 (m, 2H). |
| | ethyl 4-((6-cyclopropylimidazo[1,2-a]pyridin-2- | |
| | yl)methoxy)-6-((4-(N-hydroxycarbamimidoyl)- | |
| | 2,6-dimethylbenzyl)amino)pyrimidine-2- | |
| | carboxylate (I-81) | |
| 82 | 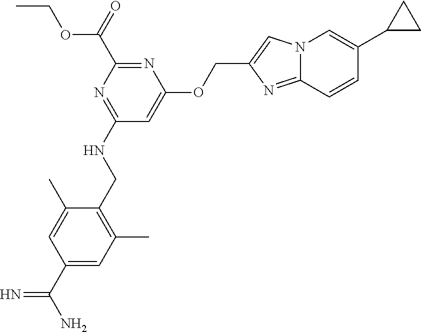 [SMILES: CCOC(=O)c1nc(NCc2c(C)cc(C(=N)N)cc2C)cc(OCc2cn3cc(C4CC4)ccc3n2)n1] | ESI-MS [M + H]⁺: 514.3. ¹H NMR (400 MHz, DMSO) δ 10.91 (s, 2H), 8.92 (s, 2H), 8.42 (s, 1H), 8.33 (s, 1H), 7.85 (s, 1H), 7.71 (s, 1H), 7.48 (s, 2H), 7.41 (d, J = 9.3 Hz, 1H), 7.00 (dd, J = 9.4, 1.7 Hz, 1H), 6.00 (s, 1H), 5.37 (s, 2H), 4.54 (s, 2H), 4.32 (q, J = 7.1 Hz, 2H), 2.45 (s, 6H), 2.00-1.86 (m, 1H), 1.33 (t, J = 7.1 Hz, 3H), 0.96-0.85 (m, 2H), 0.68 (dd, J = 5.0, 1.8 Hz, 2H). |
| | ethyl 4-((4-carbamimidoyl-2,6-dimethyl | |
| | benzyl)amino)-6-((6-cyclopropylimidazo[1,2- | |
| | a]pyridin-2-yl)methoxy)pyrimidine-2-carboxylate | |
| | formic acid salt (I-82) | |
| 83 | 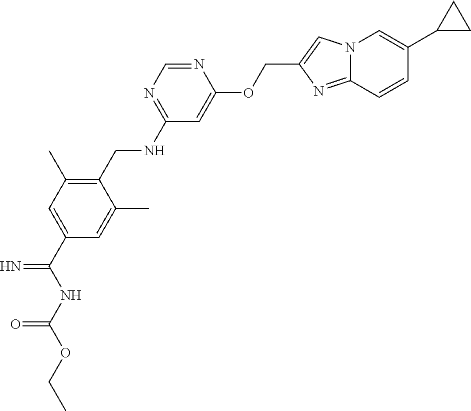 [SMILES: CCOC(=O)NC(=N)c1cc(C)c(CNc2cc(OCc3cn4cc(C5CC5)ccc4n3)ncn2)c(C)c1] | ESI-MS [M + H]⁺: 514.3. ¹H NMR (400 MHz, DMSO) δ 9.14 (s, 1H), 8.93 (s, 1H), 8.33 (s, 1H), 8.26 (s, 1H), 7.81 (s, 1H), 7.68 (s, 2H), 7.40 (d, J = 9.3 Hz, 1H), 7.22 (t, J = 4.6 Hz, 1H), 6.96- 7.03 (m, 1H), 5.86 (s, 1H), 5.35 (s, 2H), 4.48 (s, 2H), 4.01-4.09 (m, 2H), 2.38 (s, 6H), 1.88-1.97 (m, 1H), 1.22 (t, J = 7.1 Hz, 3H), 0.96-0.88 (m, 2H), 0.70- 0.64 (m, 2H). |
| | ethyl ((4-(((6-((6-cyclopropylimidazo[1,2- | |
| | a]pyridin-2-yl)methoxy)pyrimidin-4-yl) | |
| | amino)methyl)-3,5-dimethylphenyl)(imino) | |
| | methyl)carbamate (I-83) | |
| 84 | 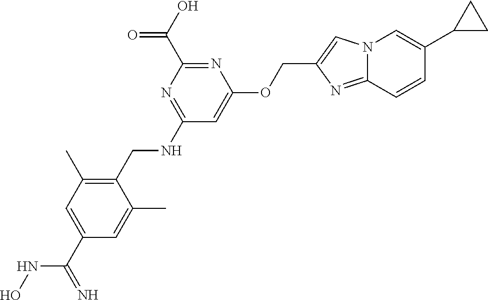 [SMILES: Cc1cc(C(=N)NO)cc(C)c1CNc1cc(OCc2cn3cc(C4CC4)ccc3n2)nc(C(=O)O)n1] | ESI-MS [M + H]⁺: 502.3. ¹H NMR (400 MHz, DMSO) δ 9.53 (s, 1H), 8.33 (s, 1H), 7.84 (s, 1H), 7.44-7.40 (m, 2H), 7.40-7.36 (m, 2H), 7.00 (dd, J = 9.4, 1.7 Hz, 1H), 5.95 (s, 1H), 5.72 (s, 2H), 5.39 (s, 2H), 4.47 (s, 2H), 2.34 (s, 6H), 2.01- 1.83 (m, 1H), 1.00-0.84 (m, 2H), 0.76-0.57 (m, 2H). |
| | 4-((6-cyclopropylimidazo[1,2-a]pyridin-2- | |
| | yl)methoxy)-6-((4-(N-hydroxycarbamimidoyl)- | |
| | 2,6-dimethylbenzyl)amino)pyrimidine-2- | |
| | carboxylic acid (I-84) | |
| 85 | 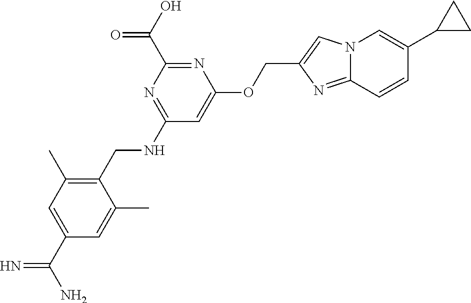 [SMILES: Cc1cc(C(=N)N)cc(C)c1CNc1cc(OCc2cn3cc(C4CC4)ccc3n2)nc(C(=O)O)n1] | ESI-MS [M + H]⁺: 486.2. ¹H NMR (400 MHz, DMSO) δ 10.58 (s, 2H), 9.07 (s, 2H), 8.32 (s, 1H), 8.27 (s, 1H), 7.84 (s, 1H), 7.49 (s, 2H), 7.40 (d, J = 9.3 Hz, 1H), 7.25 (s, 1H), 6.99 (d, J = 9.3 Hz, 1H), 5.78 (s, 1H), 5.34 (s, 2H), 4.49 (s, 2H), 2.44 (s, 6H), 1.96-1.89 (m, 1H), 1.02-0.82 (m, 2H), 0.68-0.67 (m, 2H). |
| | 4-((4-carbamimidoyl-2,6-dimethylbenzyl) | |
| | amino)-6-((6-cyclopropylimidazo[1,2-a]pyridin- | |
| | 2-yl)methoxy)pyrimidine-2-carboxylic acid | |
| | formic acid salt (I-85) | |
| 86 | 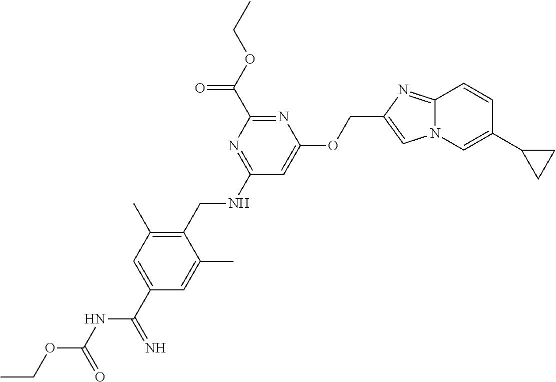 [SMILES: CCOC(=O)NC(=N)c1cc(C)c(CNc2cc(OCc3cn4cc(C5CC5)ccc4n3)nc(C(=O)OCC)n2)c(C)c1] | ESI-MS [M + H]⁺: 586.2. ¹H NMR (400 MHz, DMSO) δ 9.20-8.80 (m, 2H), 8.33 (s, 1H), 7.85 (s, 1H), 7.67-7.62 (m, 3H), 7.41 (d, J = 9.3 Hz, 1H), 7.00 (dd, J = 9.4, 1.8 Hz, 1H), 6.00 (s, 1H), 5.37 (s, 2H), 4.52 (s, 2H), 4.32 (q, J = 7.0 Hz, 2H), 4.08-4.02 (m, 2H), 2.41 (s, 6H), 2.08-1.74 (m, 1H), 1.33 (t, J = 7.0 Hz, 3H), 1.22 (t, J = 7.1 Hz, 3H), 1.01-0.85 (m, 2H), 0.77-0.66 (m, 2H). |
| | ethyl 4-((6-cyclopropylimidazo[1,2-a]pyridin-2- | |
| | yl)methoxy)-6-((4-(N-(ethoxycarbonyl) | |
| | carbamimidoyl)-2,6-dimethylbenzyl) | |
| | amino)pyrimidine-2-carboxylate (I-86) | |
| 87 | 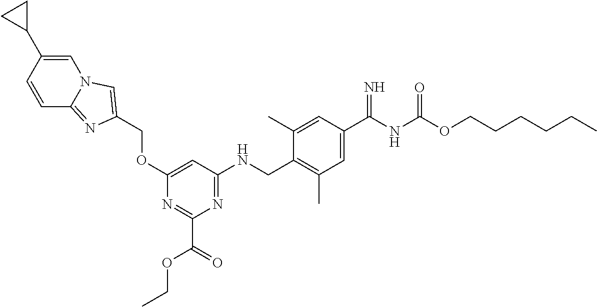 [SMILES: CCCCCCOC(=O)NC(=N)c1cc(C)c(CNc2cc(OCc3cn4cc(C5CC5)ccc4n3)nc(C(=O)OCC)n2)c(C)c1] | ESI-MS [M + H]⁺: 642.4. ¹H NMR (400 MHz, DMSO) δ 9.25-8.90 (m, 2H), 8.33 (s, 1H), 7.85 (s, 1H), 7.65 (s, 3H), 7.41 (d, J = 9.5 Hz, 1H), 6.99 (d, J = 9.2 Hz, 1H), 6.00 (s, 1H), 5.37 (s, 2H), 4.51 (s, 2H), 4.43-4.23 (m, 2H), 4.04-3.98 (m, 2H), 2.40 (s, 6H), 1.93 (s, 1H), 1.62-1.54 (m, 2H), 1.42-1.23 (m, 9H), 0.98-0.85 (m, 5H), 0.71-0.65 (m, 2H). |
| | ethyl 4-((6-cyclopropylimidazo[1,2-a]pyridin-2- | |
| | yl)methoxy)-6-((4-(N-((hexyloxy) | |
| | carbonyl)carbamimidoyl)-2,6-dimethylbenzyl) | |
| | amino)pyrimidine-2-carboxylate (I-87) | |
| 88 | 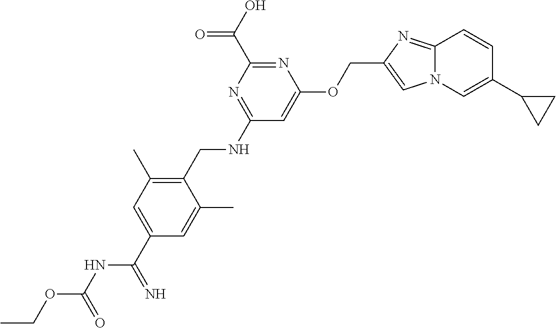 [SMILES: CCOC(=O)NC(=N)c1cc(C)c(CNc2cc(OCc3cn4cc(C5CC5)ccc4n3)nc(C(=O)O)n2)c(C)c1] | ESI-MS [M + H]⁺: 558.3. ¹H NMR (400 MHz, DMSO) δ 9.25-8.90 (m, 2H), 8.33 (s, 1H), 7.85 (s, 1H), 7.67 (s, 2H), 7.43-7.39 (m, 2H), 7.00 (d, J = 8.8 Hz, 1H), 5.90 (s, 1H), 5.38 (s, 2H), 4.49 (s, 2H), 4.08-4.03 (m, 2H), 2.39 (s, 6H), 1.96-1.89 (m, 1H), 1.22 (t, J = 7.0 Hz, 3H), 0.93-0.91 (m, 2H), 0.69-0.67 (m, 2H). |
| | 4-((6-cyclopropylimidazo[1,2-a]pyridin-2-yl) | |
| | methoxy)-6-((4-(N-(ethoxycarbonyl) | |
| | carbamimidoyl)-2,6-dimethylbenzyl)amino) | |
| | pyrimidine-2-carboxylic acid (I-88) | |
| 89 | 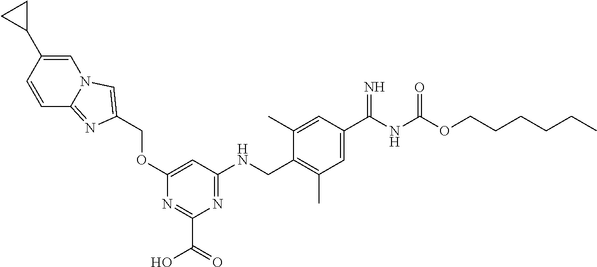 [SMILES: CCCCCCOC(=O)NC(=N)c1cc(C)c(CNc2cc(OCc3cn4cc(C5CC5)ccc4n3)nc(C(=O)O)n2)c(C)c1] | ESI-MS [M + H]⁺: 614.3. ¹H NMR (400 MHz, DMSO) δ 9.26-8.91 (m, 2H), 8.33-8.27 (m, 2H), 7.88 (s, 1H), 7.66- 7.57 (m, 2H), 7.42 (d, J = 9.3 Hz, 1H), 6.99 (d, J = 9.3 Hz, 1H), 5.84 (s, 1H), 5.38 (s, 2H), 4.39 (s, 2H), 3.99 (t, J = 6.6 Hz, 2H), 2.39 (s, 6H), 1.93 (s, 1H), 1.68-1.54 (m, 2H), 1.29 (s, 6H), 1.01- 0.81 (m, 5H), 0.68 (d, J = 5.3 Hz, 2H). |
| | 4-((6-cyclopropylimidazo[1,2-a]pyridin-2-yl) | |
| | methoxy)-6-((4-(N-((hexyloxy)carbonyl) | |
| | carbamimidoyl)-2,6-dimethylbenzyl)amino) | |
| | pyrimidine-2-carboxylic acid (I-89) | |
| 90 | 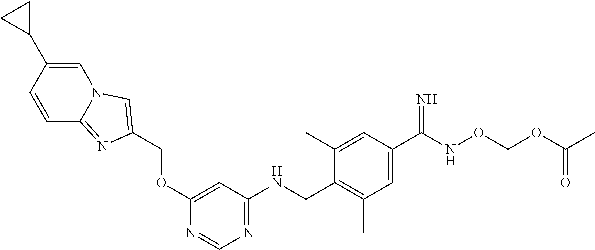 [SMILES: CC(=O)OCONC(=N)c1cc(C)c(CNc2cc(OCc3cn4cc(C5CC5)ccc4n3)ncn2)c(C)c1] | ESI-MS [M + H]⁺: 530.2. ¹H NMR (400 MHz, DMSO) δ 8.33 (s, 1H), 8.26 (s, 1H), 7.81 (s, 1H), 7.40 (d, J = 9.3 Hz, 1H), 7.35 (s, 2H), 7.17 (t, J = 4.5 Hz, 1H), 6.99 (dd, J = 9.4, 1.7 Hz, 1H), 6.21 (s, 2H), 5.85 (s, 1H), 5.60 (s, 2H), 5.35 (s, 2H), 4.45 (s, 2H), 2.34 (s, 6H), 2.05 (s, 3H), 1.96-1.90 (m, 1H), 0.95-0.86 (m, 2H), 0.72-0.64 (m, 2H). |
| | ((4-(((6-((6-cyclopropylimidazo[1,2-a]pyridin-2- | |
| | yl)methoxy)pyrimidin-4-yl)amino)methyl)-3,5- | |
| | dimethylbenzimidamido)oxy)methyl acetate (I- | |
| | 90) | |
| 91 | 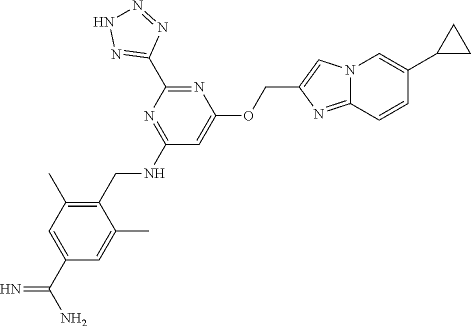 [SMILES: Cc1cc(C(=N)N)cc(C)c1CNc1cc(OCc2cn3cc(C4CC4)ccc3n2)nc(-c2nn[nH]n2)n1] | ESI-MS [M + H]⁺: 510.2. ¹H NMR (400 MHz, DMSO) δ 9.83 (s, 2H), 8.93 (s, 2H), 8.12-8.08 (m, 2H), 7.73 (s, 1H), 7.23-7.18 (m, 3H), 6.99 (s, 1H), 6.76 (dd, J = 9.4, 1.5 Hz, 1H), 5.55 (s, 1H), 5.22 (s, 2H), 4.36 (s, 2H), 2.23 (s, 6H), 1.74-1.67 (m, 1H), 0.71-0.67 (m, 2H), 0.48-0.44 (m, 2H). |
| | 4-(((6-((6-cyclopropylimidazo[1,2-a]pyridin-2- | |
| | yl)methoxy)-2-(2H-tetrazol-5-yl)pyrimidin-4- | |
| | yl)amino)methyl)-3,5-dimethylbenzimidamide | |
| | formic acid salt (I-91) | |
| 92 | 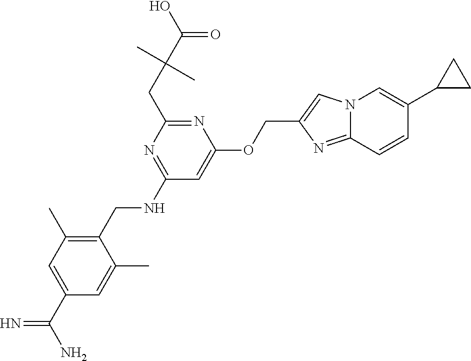 [SMILES: Cc1cc(C(=N)N)cc(C)c1CNc1cc(OCc2cn3cc(C4CC4)ccc3n2)nc(CC(C)(C)C(=O)O)n1] | ESI-MS [M + H]⁺: 542.2. ¹H NMR (400 MHz, DMSO-d₆) δ 11.67 (s, 1 H), 9.05 (s, 2 H), 8.72 (s, 2 H), 8.45 (s, 1H), 8.33 (s, 1 H), 7.82 (s, 1 H), 7.41-7.38 (m, 3 H), 7.04-7.69 (m, 2 H), 5.61 (s, 1 H), 5.30 (s, 2 H), 4.51 (s, 2 H), 2.89 (s, 2 H), 2.36 (s, 6 H), 1.96-1.90 (m, 1H), 1.15 (s, 6 H), 0.95-0.90 (m, 2H), 0.70- 0.66 (m, 2H). |
| | 3-(4-((4-carbamimidoyl-2,6-dimethylbenzyl) | |
| | amino)-6-((6-cyclopropylimidazo[1,2-a]pyridin- | |
| | 2-yl)methoxy)pyrimidin-2-yl)-2,2- | |
| | dimethylpropanoic acid formic acid salt (I-92) | |
| 93 | 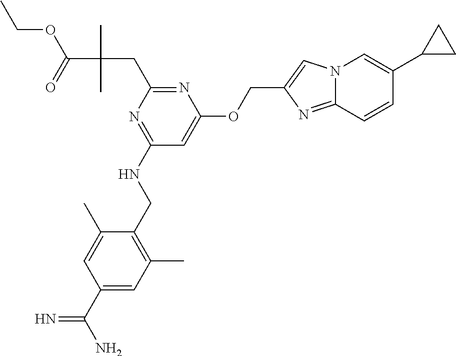 [SMILES: CCOC(=O)C(C)(C)Cc1nc(NCc2c(C)cc(C(=N)N)cc2C)cc(OCc2cn3cc(C4CC4)ccc3n2)n1] | ESI-MS [M + H]+: 570.2. ¹H NMR (400 MHz, DMSO-d₆) δ 9.35 (s, 2 H), 9.05 (s, 2 H), 8.46 (s, 1 H), 8.34 (s, 1 H), 7.79 (s, 1 H), 7.50 (s, 2 H), 7.40 (d, J = 8.0 Hz, 1 H), 7.14 (s, 1 H), 6.99 (d, J = 8.0 Hz, 1 H), 5.65 (s, 1 H), 5.27 (s, 2 H), 4.48 (s, 2 H), 4.05-4.00 (m, 3 H), 2.93 (s, 2 H), 2.40 (s, 6 H), 1.94-1.93 (m, 1H), 1.22 (s, 6 H), 1.10-1.08 (m, 3 H), 0.93-0.91 (m, 2 H), 0.68-0.67 (m, 2 H). |
| | ethyl 3-(4-((4-carbamimidoyl-2,6-dimethyl | |
| | benzyl)amino)-6-((6-cyclopropylimidazo[1,2- | |
| | a]pyridin-2-yl)methoxy)pyrimidin-2-yl)-2,2- | |
| | dimethylpropanoate formic acid salt (I-93) | |
| 94 | 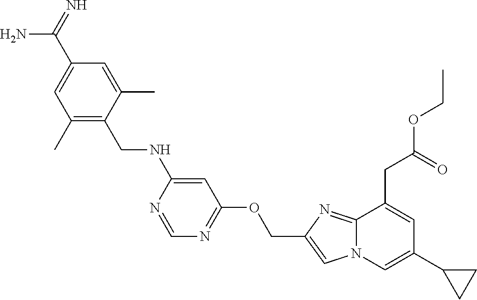 [SMILES: CCOC(=O)Cc1cc(C2CC2)cn2cc(COc3cc(NCc4c(C)cc(C(=N)N)cc4C)ncn3)nc12] | ESI-MS [M + H]⁺:: 528.3. ¹H NMR (400 MHz, DMSO) δ 9.42-8.91 (m, 2H), 8.47 (s, 1H), 8.26 (s, 2H), 7.81 (s, 1H), 7.50 (s, 2H), 7.29 (t, J = 4.7 Hz, 1H), 6.94 (s, 1H), 5.85 (s, 1H), 5.33 (s, 2H), 4.51 (s, 2H), 4.09-4.06 (m, 2H), 3.90 (s, 2H), 2.41 (s, 6H), 1.95-1.89 (m, 1H), 1.18 (t, J = 7.1 Hz, 3H), 0.96-0.85 (m, 2H), 0.72-0.58 (m, 2H). |
| | ethyl 2-(2-(((6-((4-carbamimidoyl-2,6-dimethyl | |
| | benzyl)amino)pyrimidin-4-yl)oxy)methyl)-6- | |
| | cyclopropylimidazo[1,2-a]pyridin-8-yl)acetate | |
| | formic acid salt (I-94) | |
| 95 | 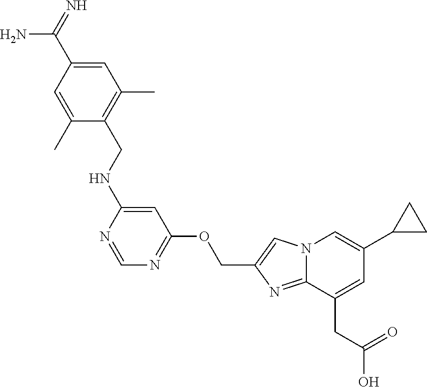 [SMILES: Cc1cc(C(=N)N)cc(C)c1CNc1cc(OCc2cn3cc(C4CC4)cc(CC(=O)O)c3n2)ncn1] | ESI-MS [M + H]⁺: 500.2. ¹H NMR (400 MHz, DMSO) δ 11.58 (s, 1H), 8.78 (s, 2H), 8.37 (s, 1H), 8.27 (s, 1H), 8.19 (s, 1H), 7.81 (s, 1H), 7.47 (s, 2H), 7.21 (s, 1H), 6.84 (s, 1H), 5.85 (s, 1H), 5.30 (s, 2H), 4.49 (s, 2H), 3.66 (s, 2H), 2.36 (s, 6H), 1.91-1.87 (m, 1H), 0.99-0.85 (m, 2H), 0.73-0.60 (m, 2H). |
| | 2-(2-(((6-((4-carbamimidoyl-2,6-dimethyl | |
| | benzyl)amino)pyrimidin-4-yl)oxy)methyl)-6- | |
| | cyclopropylimidazo[1,2-a]pyridin-8-yl)acetic | |
| | acid formic acid salt (I-95) | |
| 96 | 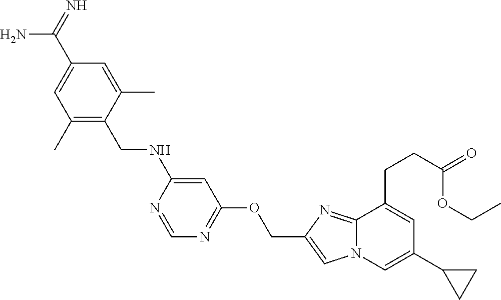 [SMILES: CCOC(=O)CCc1cc(C2CC2)cn2cc(COc3cc(NCc4c(C)cc(C(=N)N)cc4C)ncn3)nc12] | ESI-MS [M + H]⁺: 542.2. ¹H NMR (400 MHz, DMSO) δ 9.24 (s, 2H), 9.06 (s, 2H), 8.52 (s, 1H), 8.30 (s, 1H), 8.17 (s, 1H), 7.52 (s, 2H), 7.45-7.40 (m, 2H), 5.92 (s, 1H), 5.51 (s, 2H), 4.54 (s, 2H), 4.06 (q, J = 7.1 Hz, 2H), 3.15 (t, J = 7.6 Hz, 2H), 2.81 (t, J = 7.7 Hz, 2H), 2.42 (s, 6H), 2.05-1.98 (m, 1H), 1.15 (t, J = 7.1 Hz, 3H), 1.06-0.98 (m, 2H), 0.81- 0.73 (m, 2H). |
| | ethyl 3-(2-(((6-((4-carbamimidoyl-2,6-dimethyl | |
| | benzyl)amino)pyrimidin-4-yl)oxy)methyl)-6- | |
| | cyclopropylimidazo[1,2-a]pyridin-8-yl) | |
| | propanoate (I-96) | |
| 97 | 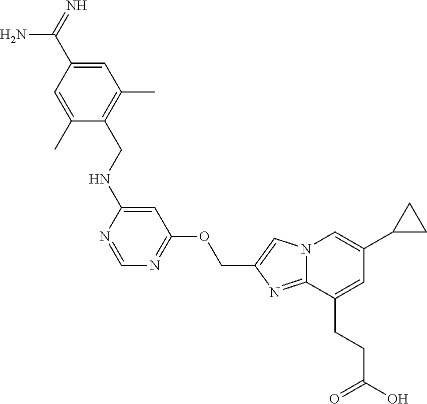 [SMILES: Cc1cc(C(=N)N)cc(C)c1CNc1cc(OCc2cn3cc(C4CC4)cc(CCC(=O)O)c3n2)ncn1] | ESI-MS [M + H]⁺: 514.2. ¹H NMR (400 MHz, DMSO) δ 12.31 (s, 1H), 9.61 (s, 2H), 9.13 (s, 2H), 8.26 (s, 1H), 8.19 (s, 1H), 7.80 (s, 1H), 7.50 (s, 2H), 7.30 (t, J = 4.7 Hz, 1H), 6.83 (s, 1H), 5.87 (s, 1H), 5.35 (s, 2H), 4.52 (s, 2H), 3.05 (t, J = 7.7 Hz, 2H), 2.69 (t, J = 7.8 Hz, 2H), 2.43 (s, 6H), 1.92-1.85 (m, 1H), 0.93-0.86 (m, 2H), 0.69-0.63 (m, 2H). |
| | 3-(2-(((6-((4-carbamimidoyl-2,6-dimethyl | |
| | benzyl)amino)pyrimidin-4-yl)oxy)methyl)-6- | |
| | cyclopropylimidazo[1,2-a]pyridin-8-yl)propanoic | |
| | acid formic acid salt (I-97) | |
| 98 | 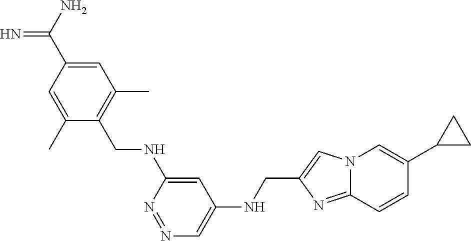 [SMILES: Cc1cc(C(=N)N)cc(C)c1CNc1cc(NCc2cn3cc(C4CC4)ccc3n2)cnn1] | ESI-MS [M + H]⁺: 441.2. ¹H NMR (400 MHz, DMSO) δ 10.93 (s, 2H), 8.93 (s, 2H), 8.33-8.30 (m, 2H), 8.07 (d, J = 2.4 Hz, 1H), 7.64 (s, 1H), 7.48 (s, 2H), 7.37 (d, J = 9.3 Hz, 1H), 7.07 (t, J = 5.6 Hz, 1H), 6.97-6.95 (m, 1H), 6.40 (s, 1H), 5.78 (d, J = 2.3 Hz, 1H), 4.50 (d, J = 4.8 Hz, 2H), 4.29 (d, J = 5.6 Hz, 2H), 2.41 (s, 6H), 1.91-1.87 (m, 1H), 0.94- 0.84 (m, 2H), 0.71-0.61 (m, 2H). |
| | 4-(((5-(((6-cyclopropylimidazo[1,2-a]pyridin-2- | |
| | yl)methyl)amino)pyridazin-3-yl)amino)methyl)- | |
| | 3,5-dimethylbenzimidamide formic acid salt | |
| | (I-98) | |
| 99 | 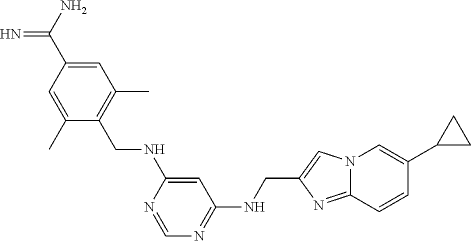 [SMILES: Cc1cc(C(=N)N)cc(C)c1CNc1cc(NCc2cn3cc(C4CC4)ccc3n2)ncn1] | ESI-MS [M + H]⁺: 441.2. ¹H NMR (400 MHz, DMSO) δ 8.44 (s, 5H), 8.29 (s, 1H), 7.99 (s, 1H), 7.58 (s, 1H), 7.48 (s, 2H), 7.35 (d, J = 9.3 Hz, 1H), 7.05 (s, 1H), 6.94 (dd, J = 9.3, 1.7 Hz, 1H), 6.74 (s, 1H), 5.50 (s, 1H), 4.40-4.43 (m, 4H), 2.40 (s, 6H), 1.98-1.83 (m, 1H), 0.97-0.84 (m, 2H), 0.74-0.59 (m, 2H). |
| | 4-(((6-(((6-cyclopropylimidazo[1,2-a]pyridin-2- | |
| | yl)methyl)amino)pyrimidin-4-yl)amino)methyl)- | |
| | 3,5-dimethylbenzimidamide formic acid salt (I- | |
| | 99) | |
| 100 | 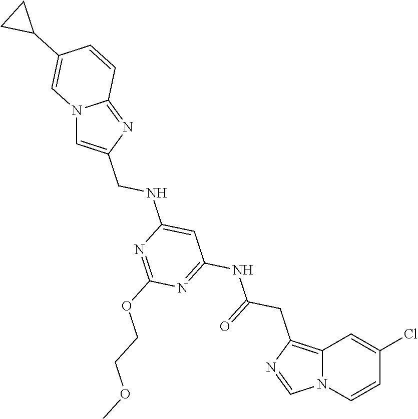 [SMILES: COCCOc1nc(NCc2cn3cc(C4CC4)ccc3n2)cc(NC(=O)Cc2ncn3ccc(Cl)cc23)n1] | ESI-MS [M + H]⁺: 547.2. ¹H NMR (400 MHz, CD₃OD) δ 8.40 (s, 1H), 8.30 (s, 1H), 8.19 (d, J = 7.1 Hz, 1H), 7.86 (s, 1H), 7.64-7.60 (m, 2H), 7.51 (d, J = 9.1 Hz, 1H), 7.02 (s, 1H), 6.64 (d, J = 7.5 Hz, 1H), 4.73 (s, 2H), 4.37 (t, J = 4.0 Hz, 2H), 3.98 (s, 2H), 3.64 (t, J = 4.0 Hz, 2H), 3.33 (s, 2H), 2.07-1.99 (m, 1H), 1.09-1.03 (m, 2H), 0.81- 0.76 (m, 2H). |
| | 2-(7-chloroimidazo[1,5-a]pyridin-1-yl)-N-(6-(((6- | |
| | cyclopropylimidazo[1,2-a]pyridin-2-yl)methyl) | |
| | amino)-2-(2-methoxyethoxy)pyrimidin-4-yl) | |
| | acetamide (I-100) | |
| 101 | 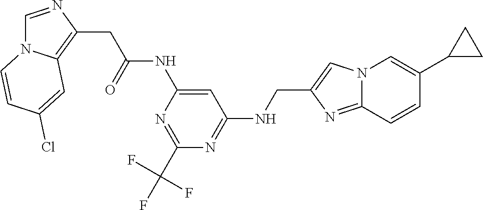 [SMILES: O=C(Cc1ncn2ccc(Cl)cc12)Nc1cc(NCc2cn3cc(C4CC4)ccc3n2)nc(C(F)(F)F)n1] | ESI-MS [M + H]⁺: 541.1. ¹H NMR (400 MHz, DMSO) δ 10.86 (s, 1H), 8.46- 8.45 (m, 1H), 8.42-8.18 (m, 3H), 7.82 (s, 1H), 7.64 (s, 1H), 7.48-7.30 (m, 2H), 6.96 (d, J = 9.3 Hz, 1H), 6.66 (d, J = 7.2 Hz, 1H), 4.62 (s, 2H), 3.97 (s, 2H), 1.93-1.87 (m, 1H), 0.91-0.85 (m, 2H), 0.68-0.63 (m, 2H). |
| | 2-(7-chloroimidazo[1,5-a]pyridin-1-yl)-N-(6-(((6- | |
| | cyclopropylimidazo[1,2-a]pyridin-2-yl)methyl) | |
| | amino)-2-(trifluoromethyl)pyrimidin-4-yl) | |
| | acetamide (I-101) | |
| 102 | 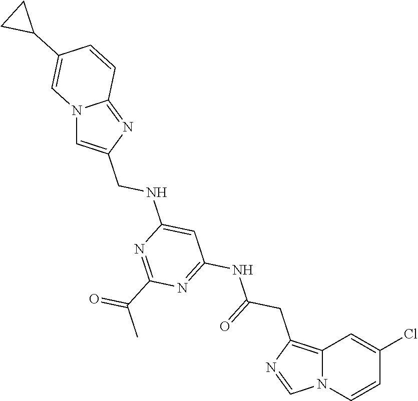 [SMILES: CC(=O)c1nc(NCc2cn3cc(C4CC4)ccc3n2)cc(NC(=O)Cc2ncn3ccc(Cl)cc23)n1] | ESI-MS [M + H]⁺: 515.2. ¹H NMR (400 MHz, DMSO) δ 10.84 (s, 1H), 8.51 (s, 1H), 8.34-8.29 (m, 3H), 7.90 (s, 1H), 7.82 (s, 1H), 7.61 (d, J = 8.4 Hz, 1H), 7.40 (s, 2H), 6.70-6.55 (m, 1H), 4.74 (s, 2H), 3.98 (s, 2H), 2.54 (s, 3H), 2.03- 1.98 (m, 1H), 1.01-0.97 (m, 2H), 0.74- 0.70 (m, 2H). |
| | N-(2-acetyl-6-(((6-cyclopropylimidazo[1,2- | |
| | a]pyridin-2-yl)methyl)amino)pyrimidin-4-yl)-2- | |
| | (7-chloroimidazo[1,5-a]pyridin-1-yl)acetamide | |
| | (I-102) | |
| 103 | 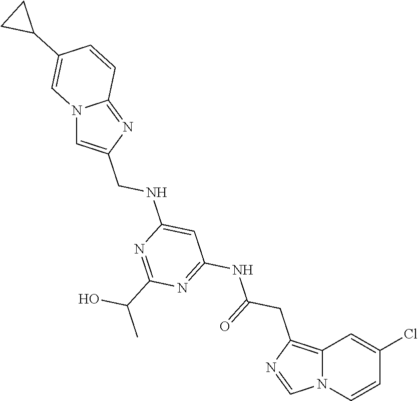 [SMILES: CC(O)c1nc(NCc2cn3cc(C4CC4)ccc3n2)cc(NC(=O)Cc2ncn3ccc(Cl)cc23)n1] | ESI-MS [M + H]⁺: 517.2. ¹H NMR (400 MHz, DMSO) δ 11.00 (s, 1H), 8.69 (s, 1H), 8.59-8.41 (m, 2H), 8.37 (d, J = 7.4 Hz, 1H), 8.08 (s, 1H), 7.89 (s, 1H), 7.79 (d, J = 9.3 Hz, 1H), 7.67 (d, J = 9.3 Hz, 1H), 7.13 (s, 1H), 6.75 (d, J = 6.5 Hz, 1H), 4.78 (s, 2H), 4.60-4.55 (m, 1H), 4.04 (s, 2H), 2.10-2.05 (m, 1H), 1.36 (d, J = 6.6 Hz, 3H), 1.07-1.02 (m, 2H), 0.81-0.77 (m, 2H). |
| | 2-(7-chloroimidazo[1,5-a]pyridin-1-yl)-N-(6-(((6- | |
| | cyclopropylimidazo[1,2-a]pyridin-2-yl)methyl) | |
| | amino)-2-(1-hydroxyethyl)pyrimidin-4-yl) | |
| | acetamide (I-103) | |
| 104 | 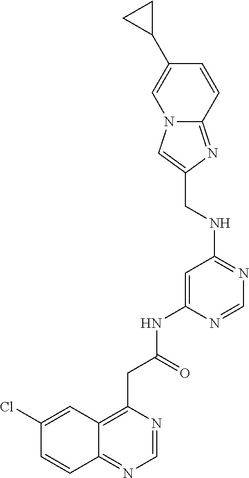 [SMILES: O=C(Cc1ncnc2ccc(Cl)cc12)Nc1cc(NCc2cn3cc(C4CC4)ccc3n2)ncn1] | ESI-MS [M + H]⁺: 485.1. ¹H NMR (400 MHz, DMSO) δ 12.56 (s, 1H), 9.88 (s, 1H), 8.41 (s, 1H), 8.30 (s, 1H), 8.19 (s, 1H), 7.97 (s, 1H), 7.81 (s, 1H), 7.67 (s, 2H), 7.62 (s, 1H), 7.45 (d, J = 9.0 Hz, 1H), 7.37 (d, J = 9.2 Hz, 1H), 7.30 (s, 1H), 6.96 (d, J = 8.1 Hz, 1H), 6.04 (s, 1H), 4.57 (s, 2H), 3.60 (s, 2H), 1.92- 1.88 (m, 1H), 0.91-0.89 (m, 2H), 0.67- 0.63 (m, 2H). |
| | 2-(6-chloroquinazolin-4-yl)-N-(6-(((6-cyclo | |
| | propylimidazo[1,2-a]pyridin-2-yl)methyl)amino) | |
| | pyrimidin-4-yl)acetamide (I-104) | |
| 105 | 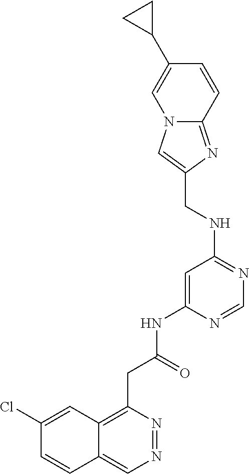 [SMILES: O=C(Cc1nncc2ccc(Cl)cc12)Nc1cc(NCc2cn3cc(C4CC4)ccc3n2)ncn1] | ESI-MS [M + H]⁺: 485.1. ¹H NMR (400 MHz, DMSO) δ 9.73-9.64 (m, 1H), 8.50-8.03 (m, 4H), 7.94-7.73 (m, 2H), 7.61 (d, J = 17.9 Hz, 1H), 7.40- 7.20 (m, 2H), 6.98-6.93 (m, 1H), 5.87 (s, 1H), 4.57 (s, 4H), 1.95-1.85 (m, 1H), 0.97-0.83 (m, 2H), 0.69-0.62 (m, 2H). |
| | 2-(7-chlorophthalazin-1-yl)-N-(6-(((6-cyclo | |
| | propylimidazo[1,2-a]pyridin-2-yl)methyl)amino) | |
| | pyrimidin-4-yl)acetamide (I-105) | |
| 106 | 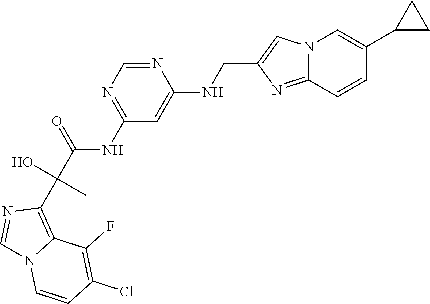 [SMILES: CC(O)(C(=O)Nc1cc(NCc2cn3cc(C4CC4)ccc3n2)ncn1)c1ncn2ccc(Cl)c(F)c12] | ESI-MS [M + H]⁺: 520.5. ¹H NMR (400 MHZ, DMSO) δ 9.60 (s, 1H), 8.30- 8.26 (m, 2H), 8.07 (d, J = 7.6 Hz, 1H), 7.99 (s, 1H), 7.60 (d, J = 9.3 Hz, 2H), 7.39-7.34 (m, 2H), 7.19 (s, 1H), 6.95 (dd, J = 9.3, 1.8 Hz, 1H), 6.82 (t, J = 7.1 Hz, 1H), 4.59 (s, 2H), 1.97 (s, 3H), 1.93-1.87 (m, 1H), 0.94-0.89 (m, 2H), 0.68-0.63 (m, 2H). |
| | 2-(7-chloro-8-fluoroimidazo[1,5-a]pyridin-1-yl)- | |
| | N-(6-(((6-cyclopropylimidazo[1,2-a]pyridin-2-yl) | |
| | methyl)amino)pyrimidin-4-yl)-2-hydroxy | |
| | propanamide (I-106) | |
| 107 | 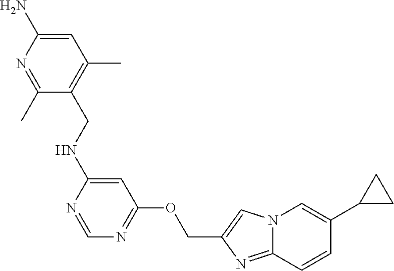 [SMILES: Cc1cc(N)nc(C)c1CNc1cc(OCc2cn3cc(C4CC4)ccc3n2)ncn1] | ESI-MS [M + H]⁺: 416.2. ¹H NMR (400 MHz, DMSO) δ 8.33 (s, 1H), 8.24 (s, 1H), 7.81 (s, 1H), 7.41 (d, J = 9.3 Hz, 1H), 7.05 (s, 1H), 7.02-6.97 (m, 1H), 6.13 (s, 1H), 5.84 (s, 1H), 5.67 (s, 2H), 5.35 (s, 2H), 4.29 (s, 2H), 2.28 (s, 3H), 2.14 (s, 3H), 1.97-1.86 (m, 1H), 0.96- 0.90 (m, 2H), 0.71-0.65 (m, 2H). |
| | N-((6-amino-2,4-dimethylpyridin-3-yl)methyl)-6- | |
| | ((6-cyclopropylimidazo[1,2-a]pyridin-2- | |
| | yl)methoxy)pyrimidin-4-amine (I-107) | |
| 108 | 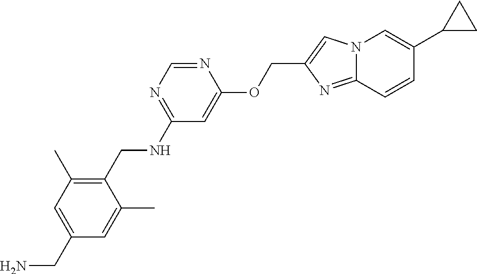 [SMILES: Cc1cc(CN)cc(C)c1CNc1cc(OCc2cn3cc(C4CC4)ccc3n2)ncn1] | ESI-MS [M + H]⁺: 429.3. ¹H NMR (400 MHz, DMSO) δ 8.33 (s, 1H), 8.25 (s, 1H), 7.80 (s, 1H), 7.40 (d, J = 9.3 Hz, 1H), 7.12-7.09 (m, 1H), 6.99-6.88 (m, 3H), 5.85 (s, 1H), 5.34 (s, 2H), 4.46- 4.38 (m, 2H), 3.63 (s, 2H), 3.25-2.96 (m, 2H), 2.30 (s, 6H), 1.97-1.88 (m, 1H), 0.96-0.88 (m, 2H), 0.71-0.61 (m, 2H). |
| | N-(4-(aminomethyl)-2,6-dimethylbenzyl)-6-((6- | |
| | cyclopropylimidazo[1,2-a]pyridin-2- | |
| | yl)methoxy)pyrimidin-4-amine (I-108) | |
| 109 | 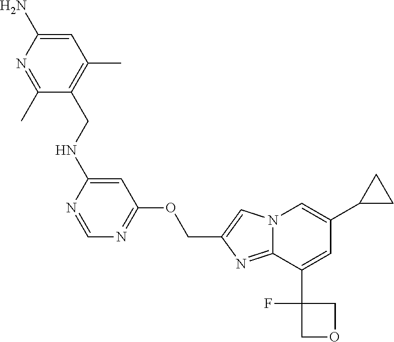 [SMILES: Cc1cc(N)nc(C)c1CNc1cc(OCc2cn3cc(C4CC4)cc(C4(F)COC4)c3n2)ncn1] | ESI-MS [M + H]⁺: 490.2. ¹H NMR (400 MHz, DMSO) δ 8.42 (s, 1H), 8.24 (s, 1H), 7.88 (s, 1H), 7.15 (s, 1H), 7.07 (s, 1H), 6.15 (s, 1H), 5.85 (s, 1H), 5.78 (s, 2H), 5.41-5.32 (m, 3H), 5.30 (d, J = 8.8 Hz, 1H), 5.00 (d, J = 8.7 Hz, 1H), 4.94 (d, J = 8.8 Hz, 1H), 4.29 (s, 2H), 2.29 (s, 3H), 2.15 (s, 3H), 1.98-1.92 (m, 1H), 0.97-0.88 (m, 2H), 0.77-0.70 (m, 2H). |
| | N-((6-amino-2,4-dimethylpyridin-3-yl)methyl)-6- | |
| | ((6-cyclopropyl-8-(3-fluorooxetan-3-yl)imidazo | |
| | [1,2-a]pyridin-2-yl)methoxy)pyrimidin-4-amine | |
| | (I-109) | |
| 110 | 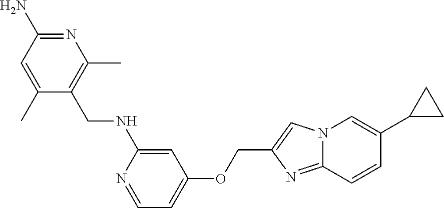 [SMILES: Cc1cc(N)nc(C)c1CNc1cc(OCc2cn3cc(C4CC4)ccc3n2)ccn1] | ESI-MS [M + H]⁺: 415.3. ¹H NMR (400 MHz, DMSO) δ 8.34 (s, 1H), 7.86- 7.78 (m, 2H), 7.41 (d, J = 9.3, 1H), 7.01- 6.88 (m, 1H), 6.23-6.20 (m, 2H), 6.18- 6.08 (m, 2H), 5.66 (s, 2H), 5.10 (s, 2H), 4.24 (d, J = 4.3, 2H), 2.29 (s, 3H), 2.15 (s, 3H), 1.98-1.87 (m, 1H), 0.95- 0.88 (m, 2H), 0.73-0.64 (m, 2H). |
| | 5-(((4-((6-cyclopropylimidazo[1,2-a]pyridin-2- | |
| | yl)methoxy)pyridin-2-yl)amino)methyl)-4,6- | |
| | dimethylpyridin-2-amine (I-110) | |
| 111 | 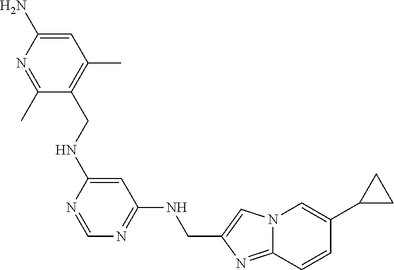 [SMILES: Cc1cc(N)nc(C)c1CNc1cc(NCc2cn3cc(C4CC4)ccc3n2)ncn1] | ESI-MS [M + H]⁺: 415.2. ¹H NMR (400 MHz, DMSO) δ 8.29 (s, 1H), 7.96 (s, 1H), 7.58 (s, 1H), 7.35 (d, J = 9.3 Hz, 1H), 7.08-6.87 (m, 2H), 6.46 (s, 1H), 6.09 (s, 1H), 5.61 (s, 2H), 5.48 (s, 1H), 4.42 (d, J = 5.1 Hz, 2H), 4.21 (d, J = 3.3 Hz, 2H), 2.25 (s, 3H), 2.12 (s, 3H), 1.93- 1.83 (m, 1H), 0.91-0.88 (m, 2H), 0.67-0.63 (m, 2H). |
| | N⁴-((6-amino-2,4-dimethylpyridin-3-yl)methyl)- | |
| | N6-((6-cyclopropylimidazo[1,2-a]pyridin-2- | |
| | yl)methyl)pyrimidine-4,6-diamine (I-111) | |
| 112 | 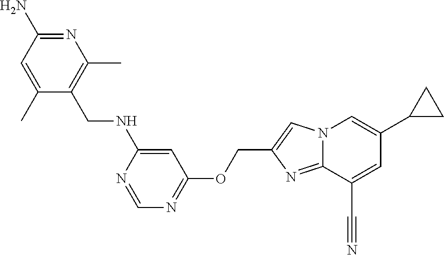 [SMILES: Cc1cc(N)nc(C)c1CNc1cc(OCc2cn3cc(C4CC4)cc(C#N)c3n2)ncn1] | ESI-MS [M + H]⁺: 441.2. ¹H NMR (400 MHz, DMSO) δ 8.68 (s, 1H), 8.26-8.22 (m, 2H), 7.98 (s, 1H), 7.78 (s, 1H), 7.06 (s, 1H), 6.12 (s, 1H), 5.85 (s, 1H), 5.67 (s, 2H), 5.41 (s, 2H), 4.29 (s, 2H), 2.27 (s, 3H), 2.14 (s, 3H), 2.04-1.92 (m, 1H), 1.08-0.89 (m, 2H), 0.78-0.74 (m,, 2H). |
| | 2-(((6-(((6-amino-2,4-dimethylpyridin-3- | |
| | yl)methyl)amino)pyrimidin-4-yl)oxy)methyl)-6- | |
| | cyclopropylimidazo[1,2-a]pyridine-8-carbonitrile | |
| | formic acid salt (I-112) | |
| 113 | 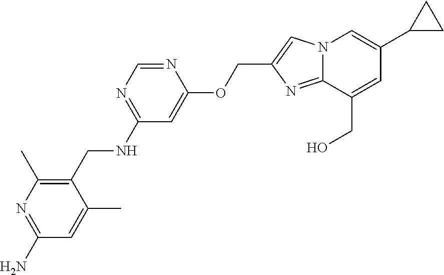 [SMILES: Cc1cc(N)nc(C)c1CNc1cc(OCc2cn3cc(C4CC4)cc(CO)c3n2)ncn1] | ESI-MS [M + H]⁺: 446.2. ¹H NMR (400 MHz, DMSO) δ 8.25-8.21 (m, 2H), 8.19 (s, 2H), 7.81 (s, 1H), 7.05-7.03 (m, 1H), 6.99 (s, 1H), 6.13 (s, 1H), 5.83 (s, 1H), 5.70 (s, 1H), 5.33 (s, 2H), 4.77 (s, 2H), 4.29-4.27 (m, 2H), 2.27 (s, 3H), 2.14 (s, 3H), 1.95-1.93 (m, 1H), 0.95-0.90 (m, 2H), 0.68-0.64 (m, 2H). |
| | (2-(((6-(((6-amino-2,4-dimethylpyridin-3- | |
| | yl)methyl)amino)pyrimidin-4-yl)oxy)methyl)-6- | |
| | cyclopropylimidazo[1,2-a]pyridin-8-yl)methanol | |
| | formic acid salt (I-113) | |
| 114 | 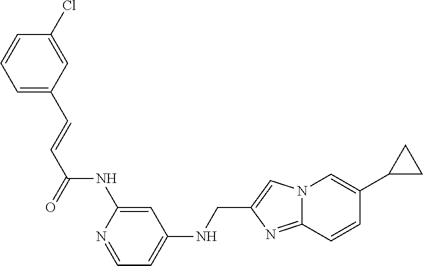 [SMILES: O=C(/C=C/c1cccc(Cl)c1)Nc1cc(NCc2cn3cc(C4CC4)ccc3n2)ccn1] | ESI-MS [M + H]⁺: 444.1. ¹H NMR (400 MHz, DMSO) δ 10.21 (s, 1H), 8.31 (s, 1H), 7.80 (d, J = 5.7 Hz, 1H), 7.65 (s, 2H), 7.60-7.51 (m, 3H), 7.51-7.43 (m, 2H), 7.38 (d, J = 9.3 Hz, 1H), 7.20 (s, 1H), 7.06 (d, J = 15.8 Hz, 1H), 6.97 (d, J = 9.3 Hz, 1H), 6.35 (d, J = 4.1 Hz, 1H), 4.38 (d, J = 5.6 Hz, 2H), 1.96- 1.85 (m, 1H), 0.97-0.85 (m, 2H), 0.71- 0.61 (m, 2H). |
| | (E)-3-(3-chlorophenyl)-N-(4-(((6- | |
| | cyclopropylimidazo[1,2-a]pyridin-2- | |
| | yl)methyl)amino)pyridin-2-yl)acrylamide (I-114) | |
| 115 | 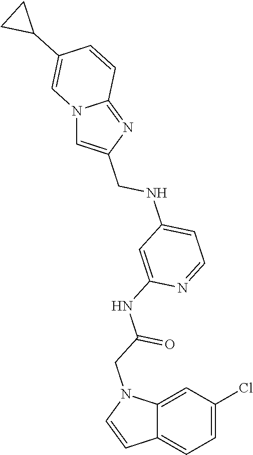 [SMILES: O=C(Cn1ccc2ccc(Cl)cc21)Nc1cc(NCc2cn3cc(C4CC4)ccc3n2)ccn1] | ESI-MS [M + H]⁺: 471.1. ¹H NMR (400 MHz, DMSO) δ 10.36 (s, 1H), 8.27 (s, 1H), 7.80 (d, J = 5.8 Hz, 1H), 7.59- 7.54 (m, 3H), 7.41 (d, J = 3.2 Hz, 1H), 7.37-7.31 (m, 2H), 7.11 (t, J = 5.8 Hz, 1H), 7.05-7.02 (m, 1H), 6.97-6.92 (m, 1H), 6.48 (d, J = 2.8 Hz, 1H), 6.386.33 (m, 1H), 5.07 (s, 2H), 4.31 (d, J = 5.7 Hz, 2H), 1.95-1.84 (m, 1H), 0.95-0.83 (m, 2H), 0.71-0.59 (m, 2H). |
| | 2-(6-chloro-1H-indol-1-yl)-N-(4-(((6- | |
| | cyclopropylimidazo[1,2-a]pyridin-2- | |
| | yl)methyl)amino)pyridin-2-yl)acetamide (I-115) | |
| 116 | 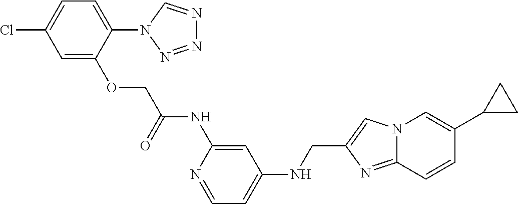 [SMILES: O=C(COc1cc(Cl)ccc1-n1cnnn1)Nc1cc(NCc2cn3cc(C4CC4)ccc3n2)ccn1] | ESI-MS [M + H]⁺: 516.2.. ¹H NMR (400 MHz, DMSO) δ 10.22 (s, 1H), 9.96 (s, 1H), 8.30 (s, 1H), 7.81 (dd, J = 14.4, 7.2 Hz, 2H), 7.63 (s, 1H), 7.45 (d, J = 1.8 Hz, 1H), 7.38-7.30 (m, 3H), 7.23- 7.16 (m, 1H), 6.96 (d, J = 9.3 Hz, 1H), 6.36 (d, J = 4.2 Hz, 1H), 5.01 (s, 2H), 4.35 (d, J = 5.5 Hz, 2H), 1.93-1.86 (m, 1H), 0.93-0.88 (m, 2H), 0.68-0.64 (m, 2H). |
| | 2-(5-chloro-2-(1H-tetrazol-1-yl)phenoxy)-N-(4- | |
| | (((6-cyclopropylimidazo[1,2-a]pyridin-2- | |
| | yl)methyl)amino)pyridin-2-yl)acetamide (I-116) | |
| 117 | 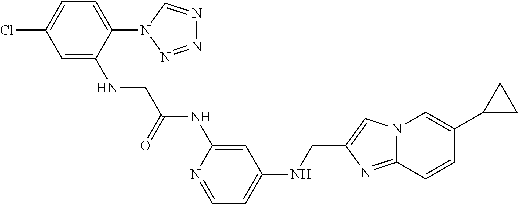 [SMILES: O=C(CNc1cc(Cl)ccc1-n1cnnn1)Nc1cc(NCc2cn3cc(C4CC4)ccc3n2)ccn1] | ESI-MS [M + H]⁺: 515.1.. ¹H NMR (400 MHz, DMSO) δ 9.99 (s, 1H), 9.78 (s, 1H), 8.29 (d, J = 0.8 Hz, 1H), 7.76 (d, J = 5.8 Hz, 1H), 7.62 (s, 1H), 7.38-7.34 (m, 3H), 7.14 (t, J = 5.8 Hz, 1H), 6.96 (dd, J = 9.3, 1.8 Hz, 1H), 6.87-6.75 (m, 2H), 6.33 (dd, J = 5.8, 2.1 Hz, 1H), 6.09 (t, J = 6.0 Hz, 1H), 4.34 (d, J = 5.7 Hz, 2H), 3.96 (d, J = 6.1 Hz, 2H), 1.92- 1.87 (m, 1H), 0.91-0.89 (m, 2H), 0.72- 0.62 (m, 2H). |
| | 2-((5-chloro-2-(1H-tetrazol-1-yl)phenyl)amino)- | |
| | N-(4-(((6-cyclopropylimidazo[1,2-a]pyridin-2- | |
| | yl)methyl)amino)pyridin-2-yl)acetamide (I-117) | |
| 118 | 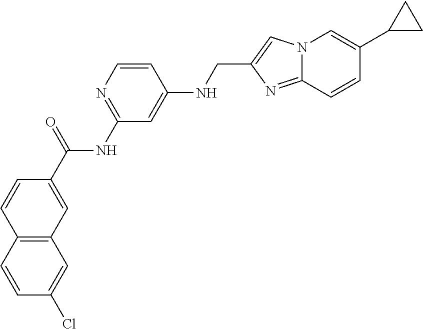 [SMILES: O=C(Nc1cc(NCc2cn3cc(C4CC4)ccc3n2)ccn1)c1ccc2ccc(Cl)cc2c1] | ESI-MS [M + H]⁺: 468.2. ¹H NMR (400 MHz, DMSO) δ 10.43 (s, 1H), 8.60 (s, 1H), 8.32 (s, 1H), 8.15 (d, J = 2.1 Hz, 1H), 8.05 (t, J = 4.4 Hz, 3H), 7.86 (d, J = 5.8 Hz, 1H), 7.71-7.57 (m, 3H), 7.39 (d, J = 9.3 Hz, 1H), 7.24 (s, 1H), 6.97 (dd, J = 9.3, 1.8 Hz, 1H), 6.42 (dd, J = 5.8, 2.1 Hz, 1H), 4.42 (d, J = 5.7 Hz, 2H), 1.97-1.83 (m, 1H), 0.98- 0.84 (m, 2H), 0.76-0.57 (m, 2H). |
| | 7-chloro-N-(4-(((6-cyclopropylimidazo[1,2- | |
| | a]pyridin-2-yl)methyl)amino)pyridin-2-yl)-2- | |
| | naphthamide (I-118) | |
| 119 | 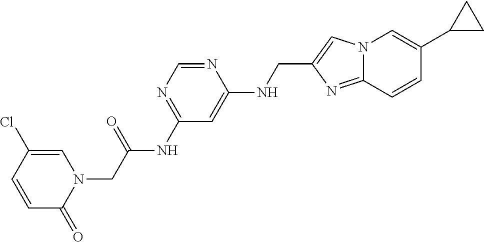 [SMILES: O=C(Cn1cc(Cl)ccc1=O)Nc1cc(NCc2cn3cc(C4CC4)ccc3n2)ncn1] | ESI-MS [M + H]⁺: 450.1. ¹H NMR (400 MHz, DMSO) δ 10.73 (s, 1H), 8.29 (s, 1H), 8.23 (s, 1H), 8.01-7.84 (m, 2H), 7.60 (s, 1H), 7.55-7.52 (m, 1H), 7.36 (d, J = 9.3 Hz, 1H), 7.16 (s, 1H), 6.96- 6.94 (m, 1H), 6.45 (d, J = 9.7 Hz, 1H), 4.75 (s, 2H), 4.58 (s, 2H), 1.93-1.86 (m, 1H), 0.92-0.88 (m, 2H), 0.67- 0.63 (m, 2H). |
| | 2-(5-chloro-2-oxopyridin-1(2H)-yl)-N-(6-(((6- | |
| | cyclopropylimidazo[1,2-a]pyridin-2- | |
| | yl)methyl)amino)pyrimidin-4-yl)acetamide (I- | |
| | 119) | |
| 120 | 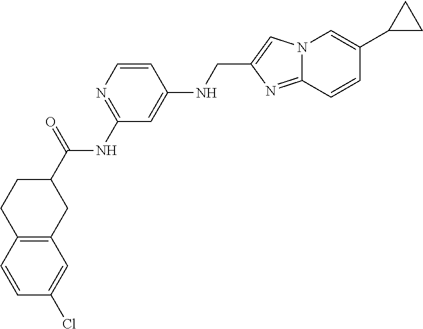 [SMILES: O=C(Nc1cc(NCc2cn3cc(C4CC4)ccc3n2)ccn1)C1CCc2ccc(Cl)cc2C1] | ESI-MS [M + H]⁺: 472.1. ¹H NMR (400 MHz, DMSO) δ 10.09 (s, 1H), 8.31 (s, 1H), 8.22 (s, 1H), 7.76 (d, J = 5.8 Hz, 1H), 7.63 (s, 1H), 7.46 (s, 1H), 7.38 (d, J = 9.3 Hz, 1H), 7.19 (d, J = 1.9 Hz, 1H), 7.16-7.07 (M, 2H), 6.99 - 6.93 (M, 1H), 6.35-6.29 (M, 1H), 4.36 (d, J = 5.6 Hz, 2H), 2.91-2.62 (m, 5H), 2.05-1.95 (m, 1H), 1.94-1.84 (m, 1H), 1.77-1.61 (m, 1H), 0.95-0.85 (M, 2H), 0.70-0.60 (m, 2H). |
| | 7-chloro-N-(4-(((6-cyclopropylimidazo[1,2- | |
| | a]pyridin-2-yl)methyl)amino)pyridin-2-yl)- | |
| | 1,2,3,4-tetrahydronaphthalene-2-carboxamide (I- | |
| | 120) | |
| 121 | 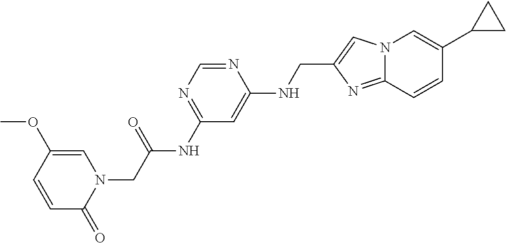 [SMILES: COc1ccc(=O)n(CC(=O)Nc2cc(NCc3cn4cc(C5CC5)ccc4n3)ncn2)c1] | ESI-MS [M + H]⁺: 446.2. ¹H NMR (400 MHz, DMSO) δ 10.66 (s, 1H), 8.29 (s, 1H), 8.23 (s, 1H), 7.89 (s, 1H), 7.60 (s, 1H), 7.37-7.32 (m, 3H), 7.17 (s, 1H), 6.96-6.94 (m, 1H), 6.39-6.36 (m, 1H), 4.71 (s, 2H), 4.57 (s, 2H), 3.62 (s, 3H), 1.92-1.86 (m, 1H), 0.92-0.88 (m, 2H), 0.67-0.63 (m, 2H). |
| | N-(6-(((6-cyclopropylimidazo[1,2-a]pyridin-2- | |
| | yl)methyl)amino)pyrimidin-4-yl)-2-(5-methoxy- | |
| | 2-oxopyridin-1(2H)-yl)acetamide (I-121) | |
| 122 | 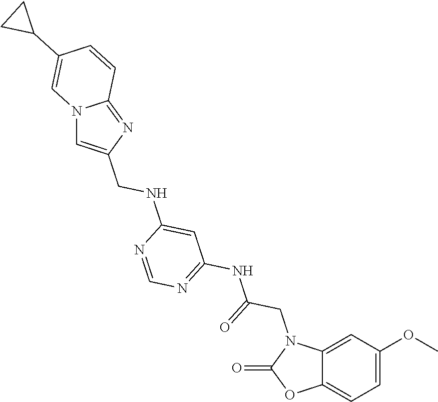 [SMILES: COc1ccc2oc(=O)n(CC(=O)Nc3cc(NCc4cn5cc(C6CC6)ccc5n4)ncn3)c2c1] | ESI-MS [M + H]⁺: 486.1. ¹H NMR (400 MHz, DMSO) δ = 10.78 (s, 1H), 8.28 (s, 1H), 8.24 (s, 1H), 7.90 (s, 1H), 7.59 (s, 1H), 7.35 (d, J = 9.4, 1H), 7.27 (d, J = 8.8, 1H), 7.16 (s, 1H), 7.02 (d, J = 2.5, 1H), 6.94 (dd, J = 9.3, 1.7, 1H), 6.67 (dd, J = 8.8, 2.6, 1H), 4.76 (s, 2H), 4.57 (s, 2H), 3.74 (s, 3H), 1.93-1.86 (m, 1H), 0.93-0.87 (m, 2H), 0.67-0.63 (m, 2H). |
| | N-(6-(((6-cyclopropylimidazo[1,2-a]pyridin-2- | |
| | yl)methyl)amino)pyrimidin-4-yl)-2-(5-methoxy- | |
| | 2-oxobenzo[d]oxazol-3(2H)-yl)acetamide (I-122) | |
| 123 | 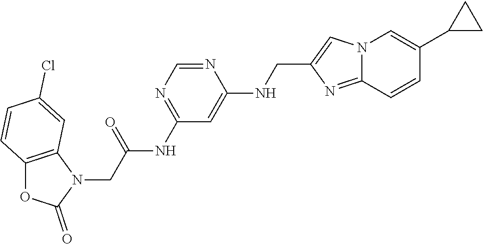 [SMILES: O=C(Cn1c(=O)oc2ccc(Cl)cc21)Nc1cc(NCc2cn3cc(C4CC4)ccc3n2)ncn1] | ESI-MS [M + H]⁺: 490.1. ¹H NMR (400 MHz, DMSO) δ 10.79 (s, 1H), 8.26 (d, J = 14.8 Hz, 2H), 7.89 (s, 1H), 7.59 (s, 1H), 7.54 (d, J = 2.1 Hz, 1H), 7.41 (d, J = 8.6 Hz, 1H), 7.35 (d, J = 9.2 Hz, 1H), 7.24-7.11 (m, 2H), 6.94 (dd, J = 9.3, 1.8 Hz, 1H), 4.78 (s, 2H), 4.57 (s, 2H), 1.96-1.83 (m, 1H), 0.97-0.85 (m, 2H), 0.65 (dt, J = 6.4, 4.4 Hz, 2H). |
| | 2-(5-chloro-2-oxobenzo[d]oxazol-3(2H)-yl)-N- | |
| | (6-(((6-cyclopropylimidazo[1,2-a]pyridin-2- | |
| | yl)methyl)amino)pyrimidin-4-yl)acetamide (I- | |
| | 123) | |
| 124 | 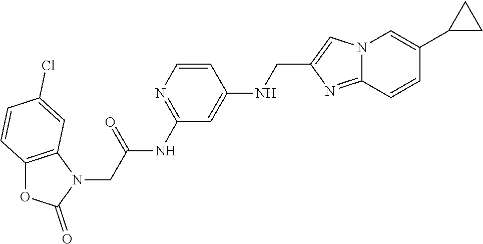 [SMILES: O=C(Cn1c(=O)oc2ccc(Cl)cc21)Nc1cc(NCc2cn3cc(C4CC4)ccc3n2)ccn1] | ESI-MS [M + H]⁺: 489.1. ¹H NMR (400 MHz, DMSO) δ 10.50 (s, 1H), 8.28 (s, 1H), 7.80 (d, J = 5.8 Hz, 1H), 7.60 (s, 1H), 7.52 (d, J = 2.1 Hz, 1H), 7.43- 7.30 (m, 3H), 7.22-7.17 (m, 1H), 7.12 (s, 1H), 6.98-6.93 (m, 1H), 6.40- 6.32 (m, 1H), 4.75 (s, 2H), 4.32 (d, J = 5.6 Hz, 2H), 1.94-1.85 (m, 1H), 0.93- 0.86 (m, 2H), 0.68-0.60 (m, 2H). |
| | 2-(5-chloro-2-oxobenzo[d]oxazol-3(2H)-yl)-N- | |
| | (4-(((6-cyclopropylimidazo[1,2-a]pyridin-2- | |
| | yl)methyl)amino)pyridin-2-yl)acetamide (I-124) | |
| 125 | 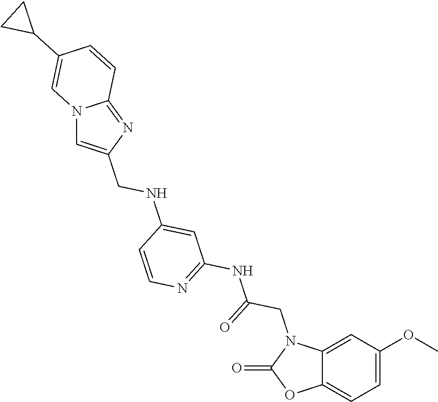 [SMILES: COc1ccc2oc(=O)n(CC(=O)Nc3cc(NCc4cn5cc(C6CC6)ccc5n4)ccn3)c2c1] | ESI-MS [M + H]⁺: 485.2. ¹H NMR (400 MHz, DMSO) δ 10.48 (s, 1H), 8.28 (s, 1H), 7.80 (d, J = 5.8 Hz, 1H), 7.61 (s, 1H), 7.38-7.33 (m, 2H), 7.26 (d, J = 8.8 Hz, 1H), 7.12 (s, 1H), 6.99 (d, J = 2.5 Hz, 1H), 6.95 (dd, J = 9.3, 1.7 Hz, 1H), 6.67 (dd, J = 8.8, 2.6 Hz, 1H), 6.36 (dd, J = 5.8, 2.0 Hz, 1H), 4.72 (s, 2H), 4.32 (d, J = 5.7 Hz, 2H), 3.74 (s, 3H), 1.93-1.85 (m, 1H), 0.91-0.84 (m, 2H), 0.67-0.63 (m, 2H). |
| | N-(4-(((6-cyclopropylimidazo[1,2-a]pyridin-2- | |
| | yl)methyl)amino)pyridin-2-yl)-2-(5-methoxy-2- | |
| | oxobenzo[d]oxazol-3(2H)-yl)acetamide (I-125) | |
| 126 | 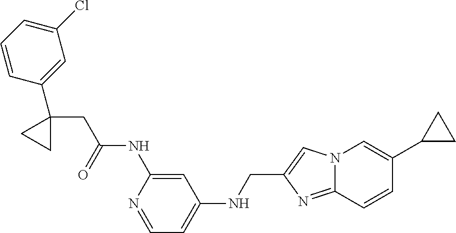 [SMILES: O=C(CC1(c2cccc(Cl)c2)CC1)Nc1cc(NCc2cn3cc(C4CC4)ccc3n2)ccn1] | ESI-MS [M + H]⁺: 472.1. ¹H NMR (400 MHz, CDCl₃) δ 8.25 (s, 1H), 7.84 (s, 1H), 7.77 (d, J = 5.9 Hz, 1H), 7.55 (s, 1H), 7.43 (d, J = 9.2 Hz, 2H), 7.31 (s, 1H), 7.25-7.10 (m, 3H), 6.96-6.90 (m, 1H), 6.38-6.16 (m, 1H), 5.12 (s, 1H), 4.50 (d, J = 5.3 Hz, 2H), 2.69 (s, 2H), 1.92-0.88 (m, 1H), 1.11-0.87 (m, 6H), 0.77-0.53 (m, 2H). |
| | 2-(1-(3-chlorophenyl)cyclopropyl)-N-(4-(((6- | |
| | cyclopropylimidazo[1,2-a]pyridin-2-yl) | |
| | methyl)amino)pyridin-2-yl)acetamide (I-126) | |
| 127 | 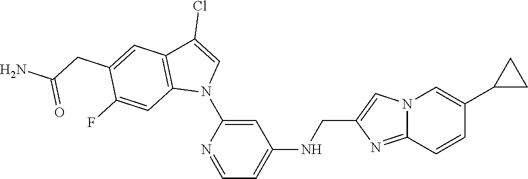 [SMILES: NC(=O)Cc1cc2c(Cl)cn(-c3cc(NCc4cn5cc(C6CC6)ccc5n4)ccn3)c2cc1F] | ESI-MS [M + H]⁺: 489.1. ¹H NMR (400 MHz, DMSO) δ 8.33 (s, 1H), 8.17- 8.12 (m, 2H), 8.00 (d, J = 5.8 Hz, 1H), 7.74 (s, 1H), 7.52-7.46 (m, 2H), 7.40 (d, J = 9.3 Hz, 1H), 7.36-7.31 (m, 1H), 6.99-6.96 (m, 2H), 6.89 (s, 1H), 6.59-6.58 (m, 1H), 4.51 (d, J = 5.5 Hz, 2H), 3.56 (s, 2H), 1.94-1.87 (m, 1H), 0.93-0.88 (m, 2H), 0.68-0.64 (m, 2H). |
| | 2-(3-chloro-1-(4-(((6-cyclopropylimidazo[1,2- | |
| | a]pyridin-2-yl)methyl)amino)pyridin-2-yl)-6- | |
| | fluoro-1H-indol-5-yl)acetamide (I-127) | |
| 128 | 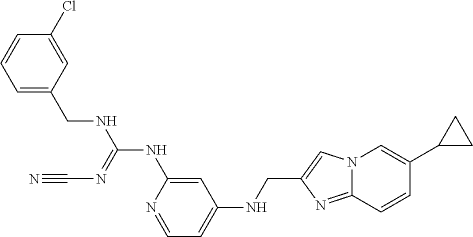 [SMILES: N#C/N=C(\NCc1cccc(Cl)c1)Nc1cc(NCc2cn3cc(C4CC4)ccc3n2)ccn1] | ESI-MS [M + H]⁺: 471.1. ¹H NMR (400 MHz, DMSO) δ 8.61-8.30 (m, 3H), 8.09-7.63 (m, 3H), 7.42-7.27 (m, 5H), 7.00 (d, J = 9.3 Hz, 1H), 6.69- 6.59 (m, J = 42.7 Hz, 1H), 6.10-5.91 (m, 1H), 4.50-4.34 (m, 4H), 1.92 (s, 1H), 1.02-0.85 (m, 2H), 0.67 (d, J = 4.5 Hz, 2H). |
| | (E)-1-(3-chlorobenzyl)-2-cyano-3-(4-(((6- | |
| | cyclopropylimidazo[1,2-a]pyridin-2-yl)methyl) | |
| | amino)pyridin-2-yl)guanidine (I-128) | |
| 129 | 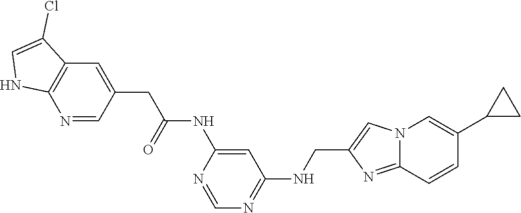 [SMILES: O=C(Cc1cnc2[nH]cc(Cl)c2c1)Nc1cc(NCc2cn3cc(C4CC4)ccc3n2)ncn1] | ESI-MS [M + H]⁺: 473.1. ¹H NMR (400 MHz, DMSO) δ 11.93 (s, 1H), 10.59 (s, 1H), 8.34-8.18 (m, 3H), 7.88 (s, 1H), 7.66 (s, 1H), 7.58 (s, 1H), 7.35 (d, J = 9.3 Hz, 1H), 7.23 (s, 1H), 6.98-6.91 (m, 1H), 4.55 (s, 2H), 3.82 (s, 2H), 1.95- 1.82 (m, 1H), 0.95-0.79 (m, 2H), 0.72-0.59 (m, 2H). |
| | 2-(3-chloro-1H-pyrrolo[2,3-b]pyridin-5-yl)-N-(6- | |
| | (((6-cyclopropylimidazo[1,2-a]pyridin-2-yl) | |
| | methyl)amino)pyrimidin-4-yl)acetamide (I-129) | |
| 130 | 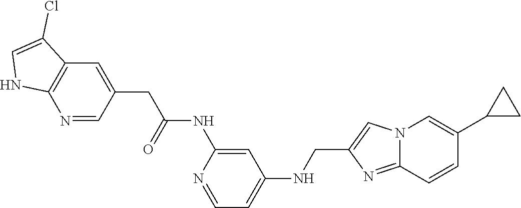 [SMILES: O=C(Cc1cnc2[nH]cc(Cl)c2c1)Nc1cc(NCc2cn3cc(C4CC4)ccc3n2)ccn1] | ESI-MS [M + H]⁺: 472.1. ¹H NMR (400 MHz, DMSO) δ 11.95 (s, 1H), 10.29 (s, 1H), 8.30-8.22 (m, 2H), 7.88 (d, J = 1.8 Hz, 1H), 7.76 (d, J = 5.8 Hz, 1H), 7.66 (s, 1H), 7.60 (s, 1H), 7.43-7.31 (m, 2H), 7.10 (t, J = 5.7 Hz, 1H), 7.00- 6.91 (m, 1H), 6.36-6.28 (m, 1H), 4.32 (d, J = 5.6 Hz, 2H), 3.78 (s, 2H), 1.95- 1.82 (m, 1H), 0.98-0.81 (m, 2H), 0.71- 0.59 (m, 2H). |
| | 2-(3-chloro-1H-pyrrolo[2,3-b]pyridin-5-yl)-N-(4- | |
| | (((6-cyclopropylimidazo[1,2-a]pyridin-2-yl) | |
| | methyl)amino)pyridin-2-yl)acetamide (I-130) | |
| 131 | 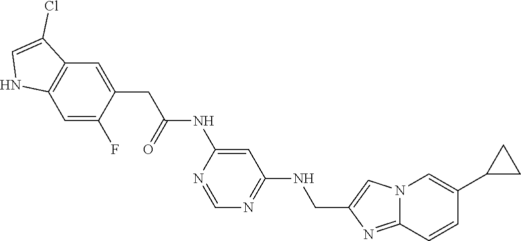 [SMILES: O=C(Cc1cc2c(Cl)c[nH]c2cc1F)Nc1cc(NCc2cn3cc(C4CC4)ccc3n2)ncn1] | ESI-MS [M + H]⁺: 490.2. ¹H NMR (400 MHz, DMSO) δ 11.38 (s, 1H), 10.50 (s, 1H), 8.28 (s, 1H), 8.21 (s, 1H), 7.85 (s, 1H), 7.59 (s, 1H), 7.51 (s, 1H), 7.45 (d, J = 6.9 Hz, 1H), 7.35 (d, J = 9.3 Hz, 1H), 7.22 (s, 2H), 6.94 (d, J = 9.3 Hz, 1H), 4.56 (s, 2H), 3.85 (s, 2H), 1.93- 1.86 (m, 1H), 0.92-0.87 (m, 2H), 0.67- 0.64 (m, 2H). |
| | 2-(3-chloro-6-fluoro-1H-indol-5-yl)-N-(6-(((6- | |
| | cyclopropylimidazo[1,2-a]pyridin-2-yl) | |
| | methyl)amino)pyrimidin-4-yl)acetamide (I-131) | |
| 132 | 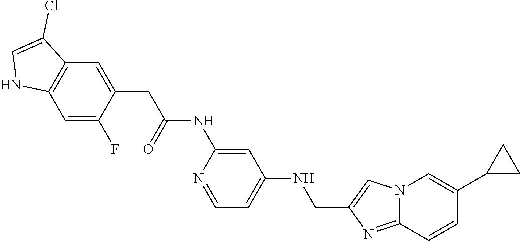 [SMILES: O=C(Cc1cc2c(Cl)c[nH]c2cc1F)Nc1cc(NCc2cn3cc(C4CC4)ccc3n2)ccn1] | ESI-MS [M + H]⁺: 489.1. ¹H NMR (400 MHz, DMSO) δ 11.36 (s, 1H), 10.17 (s, 1H), 8.28 (s, 1H), 7.77 (d, J = 5.8 Hz, 1H), 7.60 (s, 1H), 7.49 (s, 1H), 7.46 (d, J = 7.2 Hz, 1H), 7.39 (s, 1H), 7.36 (d, J = 9.3 Hz, 1H), 7.19 (d, J = 10.6 Hz, 1H), 7.10 (t, J = 5.7 Hz, 1H), 6.96- 6.94 (m, 1H), 6.33-6.31 (m, 1H), 4.32 (d, J = 5.7 Hz, 2H), 3.81 (s, 2H), 1.93- 1.86 (m, 1H), 0.92-0.83 (m, 2H), 0.69- 0.63 (m, 2H). |
| | 2-(3-chloro-6-fluoro-1H-indol-5-yl)-N-(4-(((6- | |
| | cyclopropylimidazo[1,2-a]pyridin-2-yl) | |
| | methyl)amino)pyridin-2-yl)acetamide (I-132) | |
| 133 | 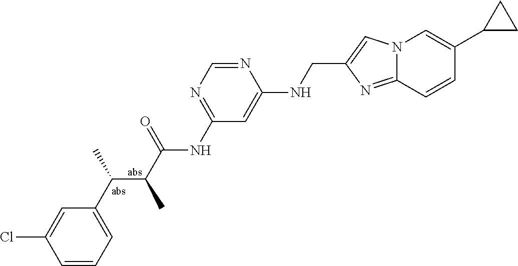 [SMILES: C[C@H](C(=O)Nc1cc(NCc2cn3cc(C4CC4)ccc3n2)ncn1)[C@@H](C)c1cccc(Cl)c1] | ESI-MS [M + H]⁺: 475.2. ¹H NMR (400 MHz, DMSO) δ 10.24 (s, 1H), 8.27 (s, 1H), 8.14 (s, 1H), 7.78 (s, 1H), 7.58 (s, 1H), 7.36-7.34 (m, 2H), 7.30-7.14 (m, 4H), 6.94 (d, J = 9.3 Hz, 1H), 4.53 (s, 2H), 3.07-3.00 (m, 1H), 2.95-2.88 (m, 1H), 1.94-1.87 (m, 1H), 1.16 (d, J = 6.9 Hz, 3H), 1.06 (d, J = 6.6 Hz, 3H), 0.91-0.88 (m, 2H), 0.65-0.64 (m, 2H). |
| | (2S,3R)-3-(3-chlorophenyl)-N-(6-(((6- | |
| | cyclopropylimidazo[1,2-a]pyridin-2-yl) | |
| | methyl)amino)pyrimidin-4-yl)-2- | |
| | methylbutanamide (I-133) | |
| 134 | 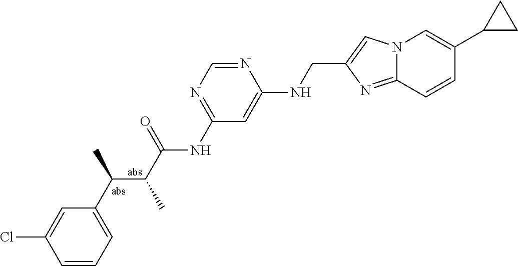 [SMILES: C[C@H](c1cccc(Cl)c1)[C@@H](C)C(=O)Nc1cc(NCc2cn3cc(C4CC4)ccc3n2)ncn1] | ESI-MS [M + H]⁺: 475.1. ¹H NMR (400 MHz, DMSO) δ 10.25 (s, 1H), 8.27 (s, 1H), 8.14 (s, 1H), 7.79 (s, 1H), 7.60 (d, J = 18.1 Hz, 1H), 7.38-7.33 (m, 2H), 7.28-7.24 (m, 1H), 7.21-7.16 (m, 3H), 6.94 (d, J = 9.4 Hz, 1H), 4.53 (s, 2H), 3.05-2.99 (m, 1H), 2.94-2.88 (m, 1H), 1.93-1.86 (m, 1H), 1.16 (d, J = 6.9 Hz, 3H), 1.06 (d, J = 6.6 Hz, 3H), 0.92-0.88 (m, 2H), 0.67-0.63 (m, 2H). |
| | (2R,3S)-3-(3-chlorophenyl)-N-(6-(((6-cyclo | |
| | propylimidazo[1,2-a]pyridin-2-yl)methyl)amino) | |
| | pyrimidin-4-yl)-2-methylbutanamide (I-134) | |
| 135 | 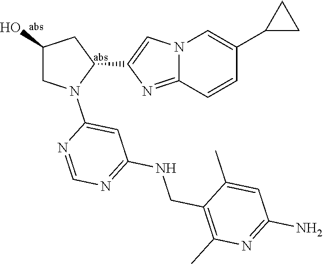 [SMILES: Cc1cc(N)nc(C)c1CNc1cc(N2C[C@@H](O)C[C@@H]2c2cn3cc(C4CC4)ccc3n2)ncn1] | ESI-MS [M + H]⁺: 471.2. ¹H NMR (400 MHz, DMSO) δ 8.22 (s, 1H), 7.97 (s, 1H), 7.51 (s, 1H), 7.35 (d, J = 9.3 Hz, 1H), 6.94 (dd, J = 9.3, 1.6 Hz, 1H), 6.43 (s, 1H), 6.07 (s, 1H), 5.61 (s, 2H), 5.36 (s, 1H), 5.05 (s, 2H), 4.45 (s, 1H), 4.20 (d, J = 29.5 Hz, 2H), 3.69 (s, 1H), 2.35- 2.16 (m, 5H), 2.07 (s, 3H), 1.92- 1.86 (m, 1H), 0.91-0.87 (m, 2H), 0.65- 0.62 (m, 2H). |
| | (3S,5R)-1-(6-(((6-amino-2,4-dimethylpyridin-3- | |
| | yl)methyl)amino)pyrimidin-4-yl)-5-(6- | |
| | cyclopropylimidazo[1,2-a]pyridin-2- | |
| | yl)pyrrolidin-3-ol (I-135) | |
| 136 | 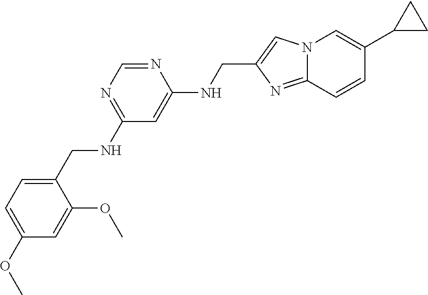 [SMILES: COc1ccc(CNc2cc(NCc3cn4cc(C5CC5)ccc4n3)ncn2)c(OC)c1] | ESI-MS [M + H]⁺: 431.2. ¹H NMR (400 MHz, DMSO) δ 8.29 (s, 1H), 7.91 (s, 1H), 7.55 (s, 1H), 7.36 (d, J = 8.0 Hz, 1H), 7.04-6.94 (m, 3H), 6.83 (t, J = 6.0 Hz, 1H), 6.52 (d, 1H), 6.41 (d, J = 8.0 Hz, 1H), 5.43 (s, 1H), 4.43 (d, J = 8.0 Hz, 2H), 4.23 (s, 2H), 3.78 (s, 3H), 3.72 (s, 3H), 1.94-1.87 (m, 1H), 0.94- 0.89 (m, 2H), 0.68-0.64 (m, 2H). |
| | N⁴-((6-cyclopropylimidazo[1,2-a]pyridin-2- | |
| | yl)methyl)-N6-(2,4- | |
| | dimethoxybenzyl)pyrimidine-4,6-diamine (I-136) | |
| 137 | 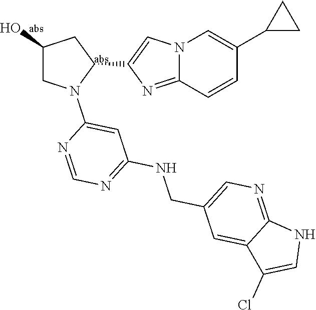 [SMILES: O[C@H]1C[C@H](c2cn3cc(C4CC4)ccc3n2)N(c2cc(NCc3cnc4[nH]cc(Cl)c4c3)ncn2)C1] | ESI-MS [M + H]⁺: 501.1. ¹H NMR (400 MHz, CD₃OD) δ 8.12 (s, 1H), 7.98 (s, 1H), 7.91 (s, 1H), 7.71 (s, 1H), 7.40- 7.33 (m, 2H), 7.23 (d, J = 9.3 Hz, 1H), 6.94 (d, J = 9.4 Hz, 1H), 5.34 (s, 1H), 5.07 (s, 1H), 4.51-4.47 (m, 3H), 3.92- 3.88 (m, 1H), 3.72 (s, 1H), 2.45- 2.41 (m, 1H), 2.29-2.25 (m, 1H), 1.90- 1.84 (m, 1H), 0.98-0.94 (m, 2H), 0.68-0.64 (m, 2H). |
| | (3S,5R)-1-(6-(((3-chloro-1H-pyrrolo[2,3- | |
| | b]pyridin-5-yl)methyl)amino)pyrimidin-4-yl)-5- | |
| | (6-cyclopropylimidazo[1,2-a]pyridin-2- | |
| | yl)pyrrolidin-3-ol (1-137) | |
| 138 | 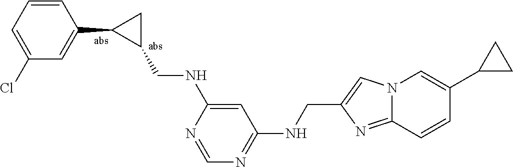 [SMILES: Clc1cccc([C@H]2C[C@@H]2CNc2cc(NCc3cn4cc(C5CC5)ccc4n3)ncn2)c1] | ESI-MS [M + H]⁺: 445.2. ¹H NMR (400 MHz, DMSO) δ 8.29 (s, 1H), 7.92 (s, 1H), 7.58 (s, 1H), 7.35 (d, J = 9.3 Hz, 1H), 7.23 (t, J = 7.8 Hz, 1H), 7.17- 7.12 (m, 1H), 7.09 (s, 1H), 7.01-6.89 (m, 3H), 6.79-6.71 (s, 1H), 5.45 (s, 1H), 4.43 (d, J = 5.3 Hz, 2H), 3.22 (s, 1H), 3.18-3.06 (m, 1H), 1.98-1.73 (m, 2H), 1.37-1.18 (m, 1H), 0.98- 0.77 (m, 4H), 0.65-0.63 (m, 3H). |
| | N⁴-(((1S,2S)-2-(3- | |
| | chlorophenyl)cyclopropyl)methyl)-N6-((6- | |
| | cyclopropylimidazo[1,2-a]pyridin-2- | |
| | yl)methyl)pyrimidine-4,6-diamine (I-138) | |
| 139 | 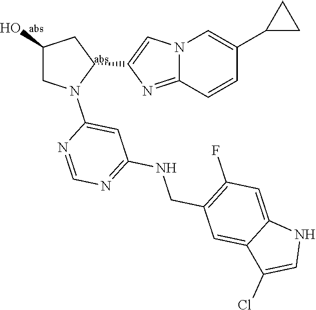 [SMILES: O[C@H]1C[C@H](c2cn3cc(C4CC4)ccc3n2)N(c2cc(NCc3cc4c(Cl)c[nH]c4cc3F)ncn2)C1] | ESI-MS [M + H]⁺: 518.2. ¹H NMR (400 MHz, CD₃OD) δ 7.97 (s, 1H), 7.89 (s, 1H), 7.36 (s, 1H), 7.24-7.15 (m, 3H), 7.01-6.92 (m, 2H), 5.34 (s, 1H), 5.06 (s, 1H), 4.46 (d, J = 34.9 Hz, 3H), 3.92- 3.89 (m, 1H), 3.73 (s, 1H), 2.45- 2.39 (m, 1H), 2.29-2.25 (m, 1H), 1.91- 1.85 (m, 1H), 1.02-0.90 (m, 2H), 0.71-0.64 (m, 2H). |
| | (3S,5R)-1-(6-(((3-chloro-6-fluoro-1H-indol-5- | |
| | yl)methyl)amino)pyrimidin-4-yl)-5-(6- | |
| | cyclopropylimidazo[1,2-a]pyridin-2-yl) | |
| | pyrrolidin-3-ol (I-139) | |
| 140 | 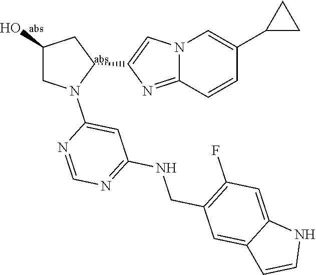 [SMILES: O[C@H]1C[C@H](c2cn3cc(C4CC4)ccc3n2)N(c2cc(NCc3cc4cc[nH]c4cc3F)ncn2)C1] | ESI-MS [M + H]⁺: 484.2. ¹H NMR (400 MHz, CD₃OD) δ 7.95 (s, 1H), 7.90 (s, 1H), 7.36 (s, 1H), 7.31-7.25 (m, 2H), 7.14 (d, J = 3.1 Hz, 1H), 7.00-6.95 (m, 2H), 6.26 (s, 1H), 5.37 (s, 1H), 5.09 (s, 1H), 4.53-4.48 (m, 1H), 4.43- 4.31 (m, 2H), 3.92-3.88 (m, 1H), 3.71 (s, 1H), 2.41-2.38 (m, 1H), 2.33- 2.28 (m, 1H), 1.92-1.86 (m, 1H), 0.99- 0.93 (m, 2H), 0.71-0.66 (m, 2H). |
| | (3S,5R)-5-(6-cyclopropylimidazo[1,2-a]pyridin- | |
| | 2-yl)-1-(6-(((6-fluoro-1H-indol-5- | |
| | yl)methyl)amino)pyrimidin-4-yl)pyrrolidin-3-ol | |
| | (I-140) | |
| 141 | 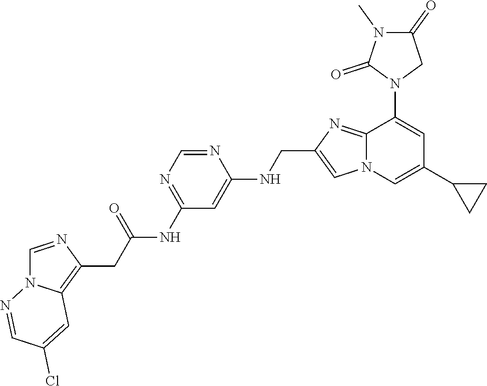 [SMILES: CN1C(=O)CN(c2cc(C3CC3)cn3cc(CNc4cc(NC(=O)Cc5ncn6ncc(Cl)cc56)ncn4)nc23)C1=O] | ESI-MS [M + H]⁺: 586.1. ¹H NMR (400 MHz, DMSO) δ 10.50 (s, 1H), 8.66 (s, 1H), 8.42 (s, 1H), 8.33 (d, J = 2.4 Hz, 1H), 8.24-8.20 (m, 2H), 7.87 (s, 1H), 7.65 (s, 1H), 7.31 (s, 1H), 7.24 (s, 1H), 4.87 (s, 2H), 4.56 (s, 2H), 3.97 (s, 2H), 2.95 (s, 3H), 1.95-1.88 (m, 1H), 0.94- 0.90 (m, 2H), 0.64-0.55 (m, 2H). |
| | 2-(3-chloroimidazo[1,5-b]pyridazin-5-yl)-N-(6- | |
| | (((6-cyclopropyl-8-(3-methyl-2,4- | |
| | dioxoimidazolidin-1-yl)imidazo[1,2-a]pyridin-2- | |
| | yl)methyl)amino)pyrimidin-4-yl)acetamide (I- | |
| | 141) | |
| 142 | 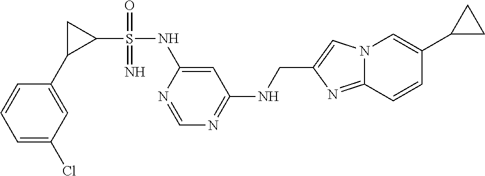 [SMILES: N=S(=O)(Nc1cc(NCc2cn3cc(C4CC4)ccc3n2)ncn1)C1CC1c1cccc(Cl)c1] | ESI-MS [M + H]⁺ 494.1, ¹H NMR (400 MHz, DMSO) δ 8.29 (s, 1H), 8.02 (s, 1H), 7.59 (s, 1H), 7.36 (d, J 9 Hz, 1H), 7.32-7.24 (m, 4H), 7.17 (dt, J 7, 2 Hz, 1H), 7.06 (br s, 2H), 6.95 (dd, J 10, 2 Hz, 1H), 5.79 (br s, 1H), 4.46 (br s, 2H), 3.16-3.12 (m, 1H), 2.79-2.74 (m, 1H), 1.93-1.87 (m, 1H), 1.65-1.54 (m, 2H), 0.93-0.88 (m, 2H), 0.67-0.63 (m, 2H). |
| | 2-(3-chlorophenyl)-N-(6-(((6- | |
| | cyclopropylimidazo[1,2-a]pyridin-2- | |
| | yl)methyl)amino)pyrimidin-4-yl)cyclopropane-1- | |
| | sulfonimidamide (I-142) | |

### Example 69

### Synthesis of 4-(((6-((6-chloroimidazo[1,2-a]pyridin-2-yl)methoxy)pyrimidin-4-yl)amino)methyl)-3,5-dimnethylbenzimidamide (I-69)

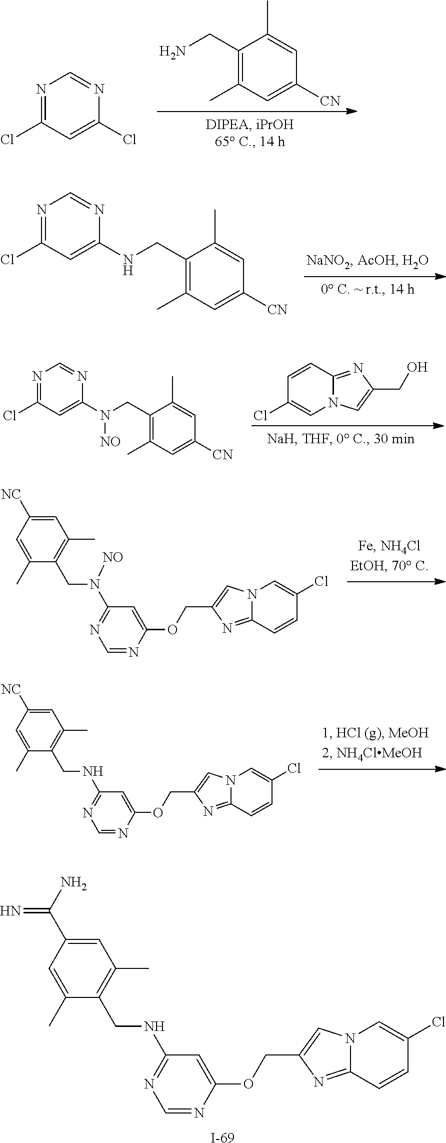

Synthesis of 4-(((6-chloropyrimidin-4-yl)amino)methyl)-3,5-dimethylbenzonitrile. A mixture of 4,6-dichloropyrimidine (1 g, 6.7 mmol), 4-(aminomethyl)-3,5-dimethylbenzonitrile (1.4 g, 8.7 mmol) and DIPEA (2.6 g, 20 mmol) in i-PrOH (40 mL) was stirred at 65° C. for 14 h. The reaction was cooled to room temperature and concentrated in vacuo to give the crude, which was purified with silica gel column chromatography, eluting with a gradient of 0-40% EtOAc in PE to give 4-(((6-chloropyrimidin-4-yl)amino)methyl)-3,5-dimethylbenzonitrile (1.4 g, yield: 77%) as a yellow solid. ESI-MS [M+H]+: 273.2.

Synthesis of N-(6-chloropyrimidin-4-yl)-N-(4-cyano-2,6-dimethylbenzyl)nitrous amide. To a solution of 4-(((6-chloropyrimidin-4-yl)amino)methyl)-3,5-dimethylbenzonitrile (1.4 g, 5.1 mmol) in AcOH (20 mL) was added a solution of NaNO₂ (1 g, 15 mmol) in water (5 mL) slowly at 0° C. The resulting reaction mixture was stirred at room temperature for 14 h. The reaction was concentrated in vacuo, the residue was neutralized with NaHCO₃ (sat. aq., 60 mL), extracted with EtOAc (60 mL×3). The combined organic layers were washed with brine (80 mL), dried over anhydrous Na₂SO₄, concentrated in vacuo to give the crude, which was purified with silica gel column chromatography, eluting with a gradient of 0-100% EtOAc in PE to give N-(6-chloropyrimidin-4-yl)-N-(4-cyano-2,6-dimethylbenzyl)nitrous amide (650 mg, yield: 42%) as a yellow solid. ESI-MS [M+H]⁺: 302.2.

Synthesis of N-(6-((6-chloroimidazo[1,2-a]pyridin-2-yl)methoxy)pyrimidin-4-yl)-N-(4-cyano-2,6-dimethylbenzyl)nitrous amide. To a solution (6-chloroimidazo[1,2-a]pyridin-2-yl)methanol (73 mg, 0.4 mmol) in THF (5 mL) was added NaH (24 mg, 0.6 mmol, 60% dispersion in mineral oil) at 0° C. and the reaction mixture was stirred at 0° C. for 5 min. Then a solution of N-(6-chloropyrimidin-4-yl)-N-(4-cyano-2,6-dimethylbenzyl)nitrous amide (100 mg, 0.33 mmol) in THF (1 mL) was added and the resulting reaction was stirred at this temperature for another 30 min. The reaction was quenched with NH₄Cl (sat. aq., 25 mL), extracted with EtOAc (30 mL×3). The combined organic layers were washed with brine (30 mL), dried over anhydrous Na₂SO₄, concentrated in vacuo to give the crude, which was purified with Prep-TLC (eluent: DCM/MeOH=20/1) to give N-(6-((6-chloroimidazo[1,2-a]pyridin-2-yl)methoxy)pyrimidin-4-yl)-N-(4-cyano-2,6-dimethylbenzyl)nitrous amide (120 mg, yield: 81%) as a yellow solid. ESI-MS [M+H]+: 447.2.

Synthesis of 4-(((6-((6-chloroimidazo[1,2-a]pyridin-2-yl)methoxy)pyrimidin-4-yl)amino)methyl)-3,5-dimethylbenzonitrile. A mixture of N-(6-((6-chloroimidazo[1,2-a]pyridin-2-yl)methoxy)pyrimidin-4-yl)-N-(4-cyano-2,6-dimethylbenzyl)nitrous amide (120 mg, 0.27 mmol), Fe (151 mg, 2.7 mmol) and NH₄Cl (143 mg, 2.7 mmol) in EtOH/H₂O (6 mL/2 mL) was stirred at 70° C. for 14 h. The reaction mixture was cooled to room temperature, filtered and the filter cake washed with MeOH (30 mL). The filtrate was concentrated in vacuo to give the crude, which was purified with Prep-TLC (eluent: DCM/MeOH=15/1) to give 4-(((6-((6-chloroimidazo[1,2-a]pyridin-2-yl)methoxy)pyrimidin-4-yl)amino)methyl)-3,5-dimethylbenzonitrile (45 mg, yield: 40%) as a yellow solid. ESI-MS [M+H]+: 419.1.

Synthesis of 4-(((6-((6-chloroimidazo[1,2-a]pyridin-2-yl)methoxy)pyrimidin-4-yl)amino)methyl)-3,5-dimethylbenzimidamide. To a solution of 4-(((6-((6-chloroimidazo[1,2-a]pyridin-2-yl)methoxy)pyrimidin-4-yl)amino)methyl)-3,5-dimethylbenzonitrile (45 mg, 0.11 mmol) in MeOH (10 mL) was bubbled dry HCl gas at room temperature for 2 h. The reaction was concentrated and the residue was re-dissolved in MeOH (5 mL), NH₄Cl (59 mg, 1.1 mmol) was added and the resulting mixture was stirred at room temperature for 16 h. The mixture was concentrated to give the crude, which was purified with Prep-HPLC to give 4-(((6-((6-chloroimidazo[1,2-a]pyridin-2-yl)methoxy)pyrimidin-4-yl)amino)methyl)-3,5-dimethylbenzimidamide as formate salt (19 mg, yield: 36%) as a white solid. ESI-MS [M+H]⁺: 436.1. 1H NMR (400 MHz, DMSO) δ9.05 (s, 1H), 8.76 (d, J=1.3 Hz, 1H), 8.39 (s, 1H), 8.21 (s, 1H), 7.88 (s, 1H), 7.52 (d, J=9.6 Hz, 1H), 7.46 (s, 2H), 7.33-7.03 (m, 2H), 5.82 (s, 1H), 5.34 (s, 2H), 4.47 (s, 2H), 2.37 (s, 6H).

### Example 70

### Synthesis of ethyl 3-(4-((4-carbamimidoyl-2,6-dimethylbenzyl)amino)-6-((6-cyclopropylimidazo[1,2-a]pyridin-2-yl)methoxy)pyrimidin-2-yl)propanoate (I-70)

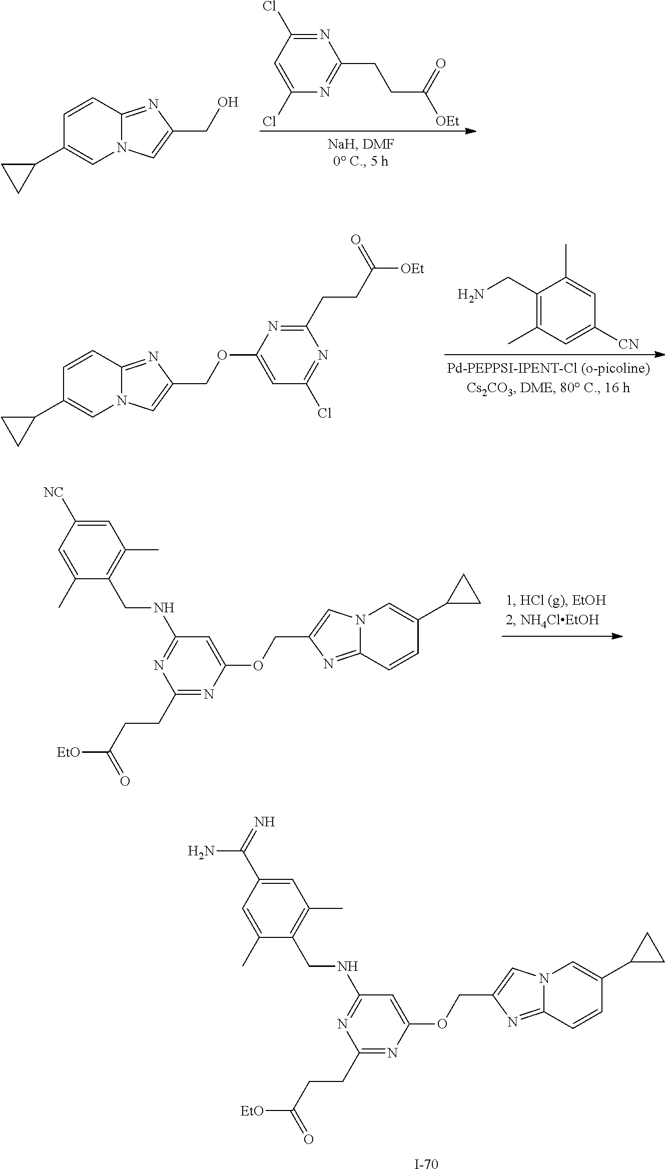

Synthesis of ethyl 3-(4-chloro-6-((6-cyclopropylimidazo[1,2-a]pyridin-2-yl)methoxy)pyrimidin-2-yl)propanoate. To a solution of (6-cyclopropylimidazo[1,2-a]pyridin-2-yl)methanol (750 mg, 4.0 mmol) in dry THF (40 mL) was added NaH (192 mg, 4.8 mmol, 60% dispersion in mineral oil) slowly at 0° C. The resulting mixture was stirred at room temperature for 20 min. Then the reaction was cooled to 0° C., a solution of ethyl 3-(4,6-dichloropyrimidin-2-yl)propanoate (1.2 g, 4.8 mmol) was added and stirred at room temperature for another 2 h. The reaction was monitored by LCMS until the starting material consumed. The reaction was quenched with HCl (aq., 1N, 6 mL), then diluted with H₂O (50 mL) and extracted with EtOAc (40 mL×3). The combined organic layers were washed with brine (60 mL), dried over Na₂SO₄, concentrated in vacuo to give the crude, which was purified with silica gel column chromatography, eluting with a gradient of 0-60% EtOAc in PE to give the ethyl 3-(4-chloro-6-((6-cyclopropylimidazo[1,2-a]pyridin-2-yl)methoxy)pyrimidin-2-yl)propanoate (350 mg, yield: 22%) as a yellow solid. ESI-MS [M+H]⁺: 401.2.

Synthesis of ethyl 3-(4-((4-cyano-2,6-dimethylbenzyl)amino)-6-((6-cyclopropylimidazo[1,2-a]pyridin-2-yl)methoxy)pyrimidin-2-yl)propanoate. To a solution of ethyl 3-(4-chloro-6-((6-cyclopropylimidazo[1,2-a]pyridin-2-yl)methoxy)pyrimidin-2-yl)propanoate (130 mg, 0.33 mmol), 4-(aminomethyl)-3,5-dimethylbenzonitrile (64 mg, 0.40 mmol), Pd-PEPPSI-IPENT-Cl (o-picoline) (17 mg, 0.02 mmol), Cs₂CO₃ (323 mg, 0.99 mmol) in DME (10 mL) was stirred at 80° C. for 16 h under N₂. The reaction was cooled to room temperature, diluted with H₂O (40 mL), extracted with EtOAc (30 mL×3). The combined organic layers were washed with brine (40 mL), dried with Na₂SO₄, concentrated in vacuo to give the crude, which was purified with combi flash chromatography with a gradient of 0-10% MeOH in DCM to give ethyl 3-(4-((4-cyano-2,6-dimethylbenzyl)amino)-6-((6-cyclopropylimidazo[1,2-a]pyridin-2-yl)methoxy)pyrimidin-2-yl)propanoate (80 mg, 46%) as a yellow solid. ESI-MS [M+H]⁺: 525.2.

Synthesis of ethyl 3-(4-((4-carbamimidoyl-2,6-dimethylbenzyl)amino)-6-((6-cyclopropylimidazo[1,2-a]pyridin-2-yl)methoxy)pyrimidin-2-yl)propanoate. To a solution of ethyl 3-(4-((4-cyano-2,6-dimethylbenzyl)amino)-6-((6-cyclopropylimidazo[1,2-a]pyridin-2-yl)methoxy)pyrimidin-2-yl)propanoate (80 mg, 0.15 mmol) in EtOH (20 mL) was bubbled HCl (g) at room temperature for 3 h. The reaction was monitored by LCMS until the starting material consumed. The reaction was concentrated to give the residue, which was re-dissolved in EtOH (20 mL), NH₄HCO₃ (119 mg, 1.5 mmol) was added to the reaction and the resulting mixture was stirred at room temperature for 14 h. The reaction was concentrated to give crude, which was purified by Prep-HPLC to give ethyl 3-(4-((4-carbamimidoyl-2,6-dimethylbenzyl)amino)-6-((6-cyclopropylimidazo[1,2-a]pyridin-2-yl)methoxy)pyrimidin-2-yl)propanoate as formate salt (50 mg, yield: 57%) as a white solid. ESI-MS [M+H]+: 542.2. 1H NMR (400 MHz, DMSO) δ9.31-9.27 (m, 2H), 9.11-8.98 (m, 2H), 8.75-8.70 (m, 1H), 8.26-8.22 (m, 1H), 7.84-7.82 (m, 1H), 7.71-7.69 (m, 1H), 7.56-7.39 (m, 3H), 5.77 (s, 1H), 5.52 (s, 2H), 4.54 (s, 2H), 4.05-4.00 (m, 2H), 2.95 (s, 2H), 2.78-2.72 (m, 2H), 2.42 (s, 6H), 2.12-2.01 (m, 1H), 1.13-1.11 (m, 3H), 1.07-1.03 (m, 2H), 0.81-0.79 (m, 2H).

### Example 71

### Synthesis of 4-(((6-((6-cyclopropylimidazo[1,2-a]pyridin-2-yl)methoxy)pyrimidin-4-yl)amino)methyl)-N-hydroxy-3,5-dimethylbenzimidamide (I-71)

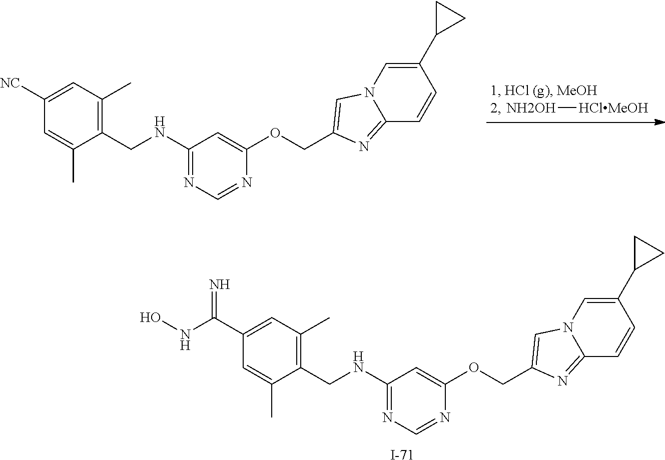

To a solution of 4-(((6-((6-cyclopropylimidazo[1,2-a]pyridin-2-yl)methoxy)pyrimidin-4-yl)amino)methyl)-3,5-dimethylbenzonitrile (200 mg, 0.47 mmol) (synthesis reported in Example 72) in MeOH (20 mL) was bubbled dry HCl gas at room temperature for 2 h. The reaction was concentrated in vacuo to give the residue, which was re-dissolved in MeOH (10 mL), then hydroxylamine hydrochloride (325 mg, 4.7 mmol) was added thereto and the reaction mixture was stirred at room temperature for 16 h. The mixture was concentrated in vacuo to give the crude, which was purified by Prep-HPLC to give 4-(((6-((6-cyclopropylimidazo[1,2-a]pyridin-2-yl)methoxy)pyrimidin-4-yl)amino)methyl)-N-hydroxy-3,5-dimethylbenzimidamide (30 mg, yield: 14%) as a white solid. ESI-MS [M+H]⁺: 458.2. ¹H NMR (400 MHz, DMSO) δ9.53 (s, 1H), 8.33 (s, 1H), 8.25 (s, 1H), 7.81 (s, 1H), 7.40 (d, J=9.3 Hz, 1H), 7.36 (s, 2H), 7.15 (t, J=4.5 Hz, 1H), 6.99 (m, 1H), 5.85 (s, 1H), 5.72 (s, 2H), 5.34 (s, 2H), 4.44 (s, 2H), 2.33 (s, 6H), 1.94-1.88 (m, 1H), 0.95-0.89 (m, 2H), 0.70-0.65 (m, 2H).

### Example 72

### Synthesis of 4-(((6-((6-cyclopropylimidazo[1,2-a]pyridin-2-yl)methoxy)pyrimidin-4-yl)amino)methyl)-3,5-dimethylbenzimidamide (I-72)

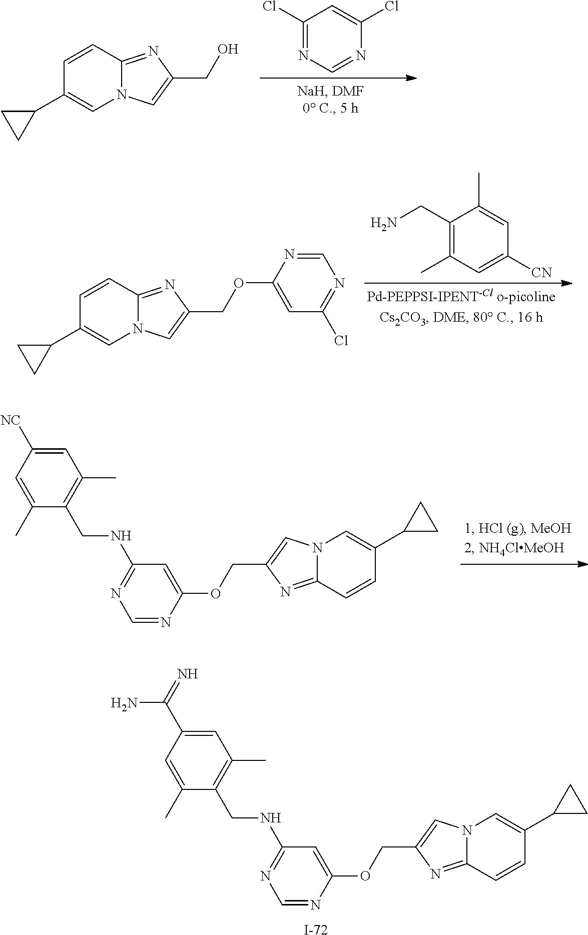

Synthesis of 2-(((6-chloropyrimidin-4-yl)oxy)methyl)-6-cyclopropylimidazo[1,2-a]pyridine. To a solution (6-cyclopropylimidazo[1,2-a]pyridin-2-yl)methanol (500 mg, 2.66 mmol) in THF (30 mL) was added NaH (128 mg, 3.2 mmol, 60% dispersion in mineral oil) at 0° C. The reaction mixture was stirred at 0° C. for 30 min. Then a solution of 4,6-dichloropyrimidine (396 mg, 2.66 mmol) in THF (5 mL) was added thereto. The resulting reaction was stirred at 0° C. for 5 h. After completed, the reaction was quenched with NH₄Cl (sat. aq., 50 mL), extracted with EtOAc (50 mL×3). The combined organic layers were washed with brine (60 mL), dried over anhydrous Na₂SO₄, concentrated in vacuo to give the crude, which was purified with silica gel column chromatography, eluting with a gradient of 0-50% EtOAc in PE to give 2-(((6-chloropyrimidin-4-yl)oxy)methyl)-6-cyclopropylimidazo[1,2-a]pyridine (460 mg, 58%) as a yellow solid. ESI-MS [M+H]+: 301.2.

Synthesis of 4-(((6-((6-cyclopropylimidazo[1,2-a]pyridin-2-yl)methoxy)pyrimidin-4-yl)amino)methyl)-3,5-dimethylbenzonitrile. A mixture of 2-(((6-chloropyrimidin-4-yl)oxy)methyl)-6-cyclopropylimidazo[1,2-a]pyridine (460 mg, 1.53 mmol), 4-(aminomethyl)-3,5-dimethylbenzonitrile hydrochloride (450 mg, 2.3 mmol), Pd-PEPPSI-IPENT^{-Cl} o-picoline (128 mg, 0.15 mmol) and Cs₂CO₃ (1.5 g, 4.6 mmol) in DME (30 mL) was stirred at 80° C. for 16 h under N₂. After cooled to room temperature, the reaction was filtered through the Celite®, washed with MeOH (50 mL). The filtrate was concentrated in vacuo to give the crude, which was purified with silica gel column chromatography, eluting with a gradient of 0-5% MeOH in DCM to give 4-(((6-((6-cyclopropylimidazo[1,2-a]pyridin-2-yl)methoxy)pyrimidin-4-yl)amino)methyl)-3,5-dimethylbenzonitrile (490 mg, 76%) as a yellow solid. ESI-MS [M+H]+: 425.2.

Synthesis of 4-(((6-((6-cyclopropylimidazo[1,2-a]pyridin-2-yl)methoxy)pyrimidin-4-yl)amino)methyl)-3,5-dimethylbenzimidamide. To a solution of 4-(((6-((6-cyclopropylimidazo[1,2-a]pyridin-2-yl)methoxy)pyrimidin-4-yl)amino)methyl)-3,5-dimethylbenzonitrile (490 mg, 1.15 mmol) in MeOH (40 mL) was bubbled dry HCl gas at room temperature for 2 h. The reaction was concentrated, and the residue was re-dissolved in MeOH (20 mL), NH₄Cl (900 mg, 17 mmol) was added and the mixture was stirred at room temperature for 16 h. The mixture was concentrated to give the crude, which was purified with Prep-HPLC to give 4-(((6-((6-cyclopropylimidazo[1,2-a]pyridin-2-yl)methoxy)pyrimidin-4-yl)amino)methyl)-3,5-dimethylbenzimidamide as formate salt (190 mg, yield: 34%) as a white solid. ESI-MS [M+H]⁺: 442.2. 1H NMR (400 MHz, DMSO) δ11.16 (s, 2H), 8.92 (s, 1H), 8.42 (s, 1H), 8.33 (s, 1H), 8.26 (s, 1H), 7.81 (s, 1H), 7.50 (s, 2H), 7.40 (d, J=9.3 Hz, 1H), 7.30 (t, J=4.7 Hz, 1H), 6.99 (dd, J=9.3, 1.6 Hz, 1H), 5.86 (s, 1H), 5.35 (s, 2H), 4.52 (s, 2H), 2.42 (s, 6H), 2.07-1.62 (m, 1H), 1.05-0.83 (m, 2H), 0.79-0.42 (m, 2H).

### Example 73

### Synthesis of ethyl 3-(4-((6-cyclopropylimidazo[1,2-a]pyridin-2-yl)methoxy)-6-((4-(N-hydroxycarbamimidoyl)-2,6-dimethylbenzyl)amino)pyrimidin-2-yl) propanoate (I-73)

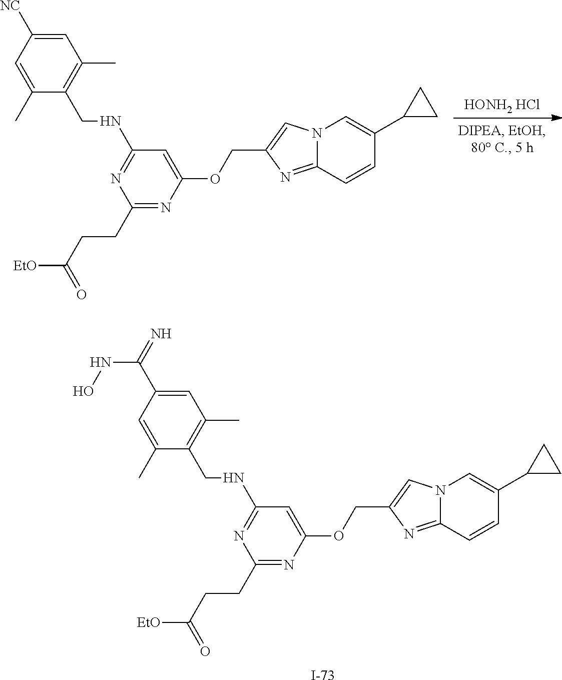

To a mixture of ethyl 3-(4-((4-cyano-2,6-dimethylbenzyl)amino)-6-((6-cyclopropylimidazo[1,2-a]pyridin-2-yl)methoxy)pyrimidin-2-yl)propanoate (80 mg, 0.15 mmol), hydroxylamine hydrochloride (31 mg, 0.45 mmol) and DIPEA (97 mg, 0.75 mmol) in EtOH (10 mL) was stirred at 80° C. for 5 h. The mixture was cooled to room temperature, water (40 mL) was added and extracted with EtOAc (30 mL×3). The combined organic layers were washed with brine (40 mL), dried with Na₂SO₄, concentrated in vacuo to give the residue, which was purified with silica gel chromatography, eluting with a gradient of 0-10% MeOH in DCM to give ethyl 3-(4-((6-cyclopropylimidazo[1,2-a]pyridin-2-yl)methoxy)-6-((4-(N-hydroxycarbamimidoyl)-2,6-dimethylbenzyl)amino)pyrimidin-2-yl)propanoate (26 mg, 31%) as a white solid. ESI-MS [M+H]⁺: 558.2. ¹H NMR (400 MHz, DMSO) δ9.52 (s, 1H), 8.33 (s, 1H), 7.80 (s, 1H), 7.40 (d, J=9.3 Hz, 1H), 7.35 (s, 2H), 7.06-7.02 (m, 1H), 7.01-6.98 (m, 1H), 5.71 (s, 2H), 5.67 (s, 1H), 5.32 (s, 2H), 4.43 (s, 2H), 4.06-4.01 (m, 2H), 2.95-2.92 (m, 2H), 2.76-2.71 (m, 2H), 2.32 (s, 6H), 1.96-1.89 (m, 1H), 1.13 (t, J=7.1 Hz, 3H), 0.94-0.90 (m, 2H), 0.71-0.63 (m, 2H).

### Example 74

### Synthesis of 3-(4-((4-carbamimidoyl-2,6-dimethylbenzyl)amino)-6-((6-cyclopropylimidazo[1,2-a]pyridin-2-yl)methoxy)pyrimidin-2-yl)propanoic acid (I-74)

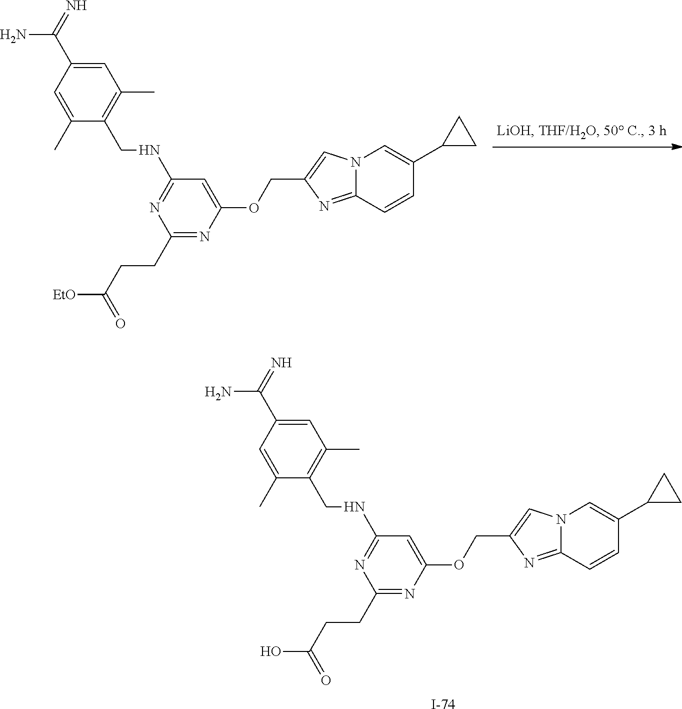

To a solution of ethyl 3-(4-((4-carbamimidoyl-2,6-dimethylbenzyl)amino)-6-((6-cyclopropylimidazo[1,2-a]pyridin-2-yl)methoxy)pyrimidin-2-yl)propanoate (40 mg, 0.074 mmol) and LiOH—H₂O (12 mg, 0.30 mmol) in THF/H₂O (2 mL/2 mL) was stirred at 50° C. for 3 h. The reaction was cooled to room temperature and concentrated in vacuo to give the crude, which was purified with Prep-HPLC to give 3-(4-((4-carbamimidoyl-2,6-dimethylbenzyl)amino)-6-((6-cyclopropylimidazo[1,2-a]pyridin-2-yl)methoxy)pyrimidin-2-yl)propanoic acid as formate salt (20 mg, 48%) as a white solid. ESI-MS [M+H]⁺: 514.2. 1H NMR (400 MHz, DMSO) δ11.31-11.20 (m, 2H), 9.08-8.73 (m, 1H), 8.44 (s, 1H), 8.32 (s, 1H), 7.82 (s, 1H), 7.44-6.98 (m, 3H), 7.16 (s, 1H), 6.99 (d, J=9.4 Hz, 1H), 5.64 (s, 1H), 5.33 (s, 2H), 4.54 (s, 2H), 2.86-2.82 (m, 2H), 2.67-2.62 (m, 2H), 2.41 (s, 6H), 1.93-1.91 (m, 1H), 0.93-0.91 (m, 2H), 0.69-0.67 (m, 2H).

### Example 75

### Synthesis of ethyl 3-(4-((6-cyclopropylimidazo[1,2-a]pyridin-2-yl)methoxy)-6-((4-(N-((hexyloxy)carbonyl)carbamimidoyl)-2,6-dimethylbenzyl)amino)pyrimidin-2-yl)propanoate (I-75)

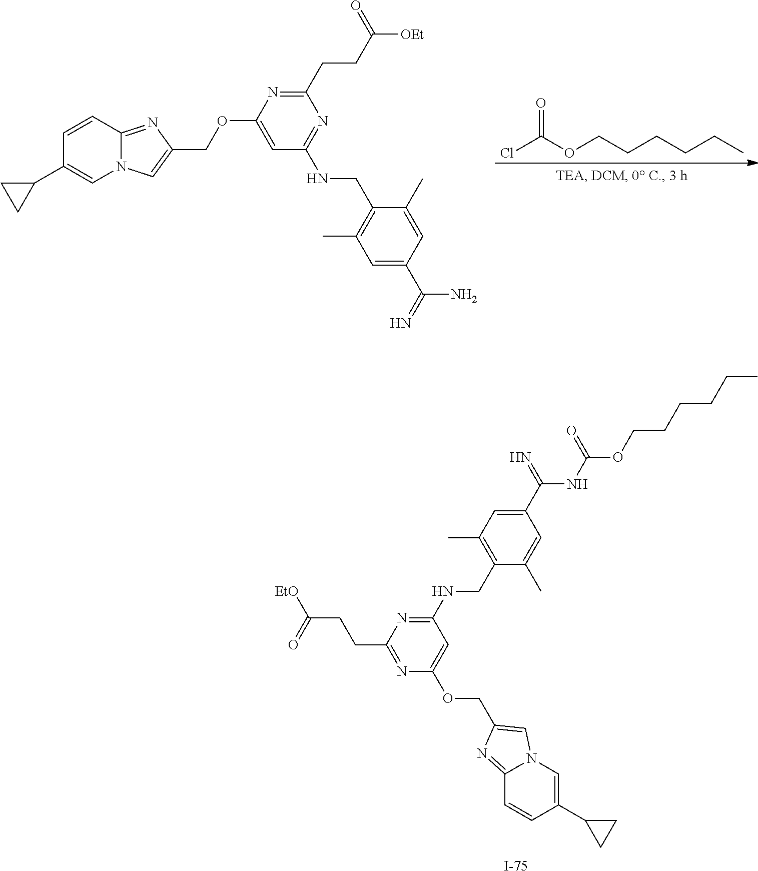

To a mixture of ethyl 3-(4-((4-carbamimidoyl-2,6-dimethylbenzyl)amino)-6-((6-cyclopropylimidazo[1,2-a]pyridin-2-yl)methoxy)pyrimidin-2-yl)propanoate (40 mg, 0.074 mmol), TEA (22 mg, 0.22 mmol) in DCM (10 mL) was added hexyl carbonochloridate (36 mg, 0.22 mmol) at 0° C. The mixture was stirred at 0° C. for 3 h then warmed to room temperature, H₂O (20 mL) was added and extracted with DCM (20 mL×3). The combined organic layers were washed with brine (30 mL), dried with Na₂SO₄, concentrated in vacuo to give the crude, which was purified with column chromatography, eluting with a gradient of 0-10% MeOH in DCM to give ethyl 3-(4-((6-cyclopropylimidazo[1,2-a]pyridin-2-yl)methoxy)-6-((4-(N-((hexyloxy)carbonyl)carbamimidoyl)-2,6-dimethylbenzyl)amino)pyrimidin-2-yl)propanoate (20 mg, 40%) as a white solid. ESI-MS [M+H]⁺: 670.3. ¹H NMR (400 MHz, DMSO) δ9.13-8.94 (m, 2H), 8.33 (s, 1H), 7.80 (s, 1H), 7.66 (s, 2H), 7.40 (d, J=9.3 Hz, 1H), 7.12 (t, J=4.6 Hz, 1H), 7.00-6.97 (m, 1H), 5.67 (s, 1H), 5.32 (s, 2H), 4.48 (s, 2H), 4.11-3.94 (m, 4H), 2.95-2.92 (m, 2H), 2.75-2.72 (m, 2H), 2.38 (s, 6H), 1.95-1.89 (m, 1H), 1.65-1.53 (m, 2H), 1.41-1.27 (m, 6H), 1.13 (t, J=7.1 Hz, 3H), 0.97-0.82 (m, 5H), 0.73-0.62 (m, 2H).

### Example 76

### Synthesis of hexyl ((4-(((6-((6-cyclopropylimidazo[1,2-a]pyridin-2-yl)methoxy)pyrimidin-4-yl)amino)methyl)-3,5-dimethylphenyl)(imino)methyl)carbamate (I-76)

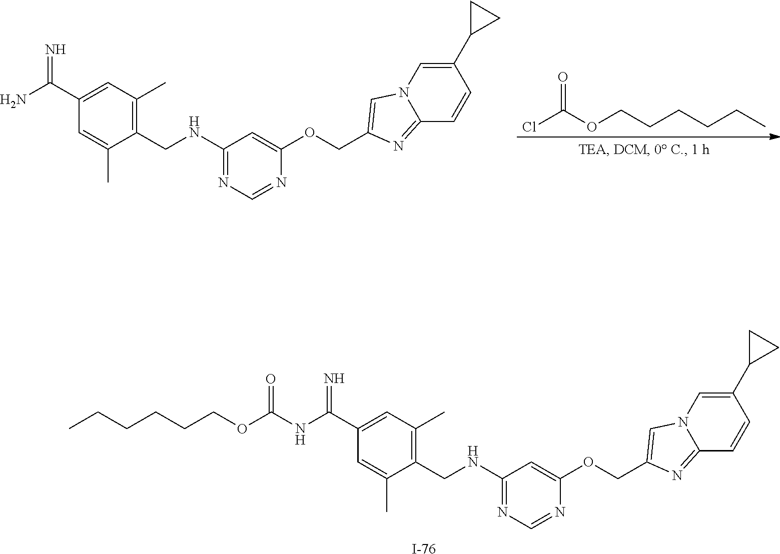

To a mixture of 4-(((6-((6-cyclopropylimidazo[1,2-a]pyridin-2-yl)methoxy)pyrimidin-4-yl)amino)methyl)-3,5-dimethylbenzimidamide (125 mg, 0.28 mmol) (synthesis reported in Example 72) and TEA (141 mg, 1.4 mmol) in DCM (10 mL) was added hexyl carbonochloridate (92 mg, 0.56 mmol) and the mixture was stirred at 0° C. for 1 h. H₂O (30 mL) was added and extracted with DCM (30 mL×3). The combined organic layers were washed with brine (30 mL), dried with Na₂SO₄, concentrated in vacuo to give the crude, which was purified by Prep-HPLC to give hexyl ((4-(((6-((6-cyclopropylimidazo[1,2-a]pyridin-2-yl)methoxy)pyrimidin-4-yl)amino)methyl)-3,5-dimethylphenyl)(imino)methyl)carbamate (38 mg, yield: 24%) as a white solid. ESI-MS [M+H]⁺: 570.3. ¹H NMR (400 MHz, DMSO) δ9.15 (s, 1H), 8.94 (s, 1H), 8.33 (s, 1H), 8.26 (s, 1H), 7.81 (s, 1H), 7.67 (s, 2H), 7.40 (d, J=9.3 Hz, 1H), 7.22 (t, J=4.6 Hz, 1H), 7.01-6.97 (m, 1H), 5.86 (s, 1H), 5.35 (s, 2H), 4.48 (s, 2H), 4.00 (t, J=6.7 Hz, 2H), 2.38 (s, 6H), 1.97-1.88 (m, 1H), 1.62-1.55 (m, 2H), 1.41-1.17 (m, 6H), 1.00-0.78 (m, 5H), 0.74-0.60 (m, 2H).

### Example 77

### Synthesis of ethyl 3-(4-((6-cyclopropylimidazo[1,2-a]pyridin-2-yl)methoxy)-6-((4-(N-(ethoxycarbonyl)carbamimidoyl)-2,6-dimethylbenzyl)amino)pyrimidin-2-yl)propanoate (I-77)

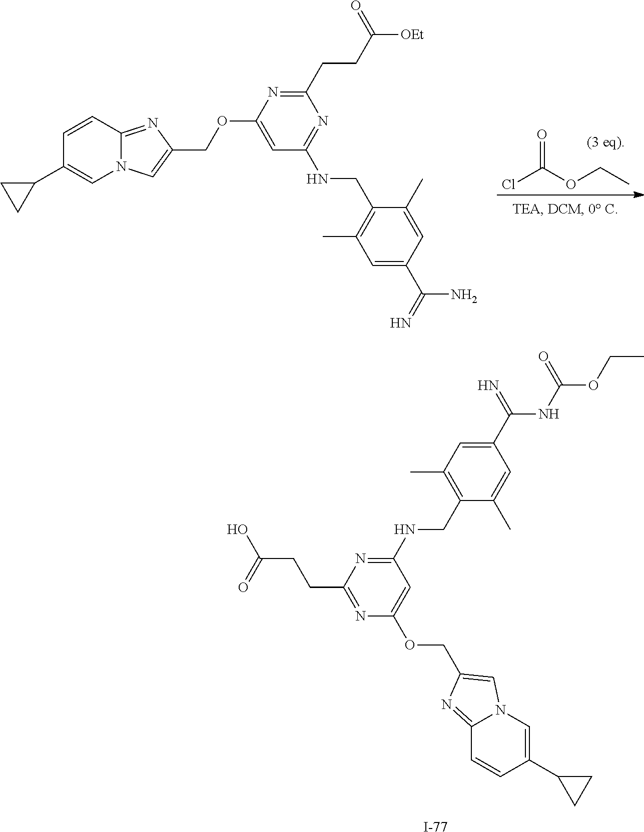

To a mixture of ethyl 3-(4-((4-carbamimidoyl-2,6-dimethylbenzyl)amino)-6-((6-cyclopropylimidazo[1,2-a]pyridin-2-yl)methoxy)pyrimidin-2-yl)propanoate (40 mg, 0.074 mmol) (synthesis reported in Example 70), TEA (22 mg, 0.22 mmol) in DCM (10 mL) was added ethyl carbonochloridate (24 mg, 0.22 mmol) at 0° C. The mixture was stirred at 0° C. for 3 h. The resulting mixture was quenched with H₂O (20 mL) and extracted with DCM (20 mL×3). The combined organic layers were washed with brine (20 mL), dried with Na₂SO₄, concentrated in vacuo to give the crude, which was purified with column chromatography with DCM/MeOH (10/1) to give ethyl 3-(4-((6-cyclopropylimidazo[1,2-a]pyridin-2-yl)methoxy)-6-((4-(N-(ethoxycarbonyl)carbamimidoyl)-2,6-dimethylbenzyl)amino)pyrimidin-2-yl)propanoate (25 mg, yield: 55%) as a white solid. ESI-MS [M+H]⁺: 614.3. ¹H NMR (400 MHz, DMSO) δ9.14-8.95 (m, 2H), 8.33 (s, 1H), 7.80 (s, 1H), 7.67 (s, 2H), 7.40 (d, J=9.3 Hz, 1H), 7.13-7.11 (m, 1H), 7.01-6.97 (m, 1H), 5.67 (s, 1H), 5.32 (s, 2H), 4.48 (s, 2H), 4.09-3.99 (m, 4H), 2.94-2.91 (m, 2H), 2.76-2.67 (m, 2H), 2.38 (s, 6H), 1.96-1.89 (m, 1H), 1.33-1.29 (m, 3H), 1.15-1.11 (m, 3H), 0.95-0.86 (m, 2H), 0.71-0.62 (m, 2H).

### Example 78

### Synthesis of 3-(4-((6-cyclopropylimidazo[1,2-a]pyridin-2-yl)methoxy)-6-((4-(N-hydroxycarbamimidoyl)-2,6-dimethylbenzyl)amino)pyrimidin-2-yl)propanoic acid (I-78)

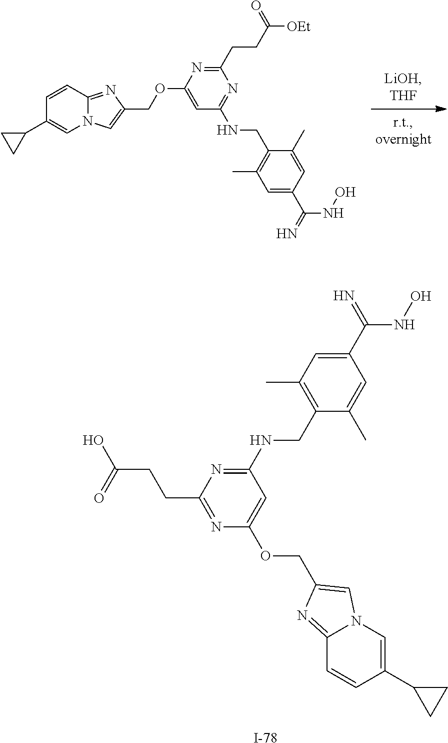

A solution of ethyl 3-(4-((6-cyclopropylimidazo[1,2-a]pyridin-2-yl)methoxy)-6-((4-(N-hydroxycarbamimidoyl)-2,6-dimethylbenzyl)amino)pyrimidin-2-yl)propanoate (15 mg, 0.027 mmol) (synthesis reported in Example 73) and LiOH—H₂O (4.4 mg, 0.11 mmol) in THF/EtOH (2 mL/2 mL) was stirred at room temperature for 8 h. The resulting mixture was concentrated to give the crude, which was purified with Prep-HPLC to give 3-(4-((6-cyclopropylimidazo[1,2-a]pyridin-2-yl)methoxy)-6-((4-(N-hydroxycarbamimidoyl)-2,6-dimethylbenzyl)amino)pyrimidin-2-yl)propanoic acid as formate salt (4.5 mg, 29% yield) as a white solid. ESI-MS [M+H]⁺: 530.2. ¹H NMR (400 MHz, DMSO) δ9.52 (s, 1H), 8.32 (s, 1H), 8.20 (s, 1H), 7.81 (s, 1H), 7.40 (d, J=9.3 Hz, 1H), 7.35 (s, 2H), 7.06-6.95 (m, 2H), 5.71 (s, 2H), 5.66 (s, 1H), 5.32 (s, 2H), 4.44 (s, 2H), 2.90-2.87 (m, 2H), 2.71-2.64 (m, 2H), 2.33 (s, 6H), 1.96-1.90 (m, 1H), 0.95-0.88 (m, 2H), 0.71-0.65 (m, 2H).

### Example 79

### Synthesis of 3-(4-((6-cyclopropylimidazo[1,2-a]pyridin-2-yl)methoxy)-6-((4-(N-((hexyloxy)carbonyl)carbamimidoyl)-2,6-dimethylbenzyl)amino)pyrimidin-2-yl)propanoic acid (I-79)

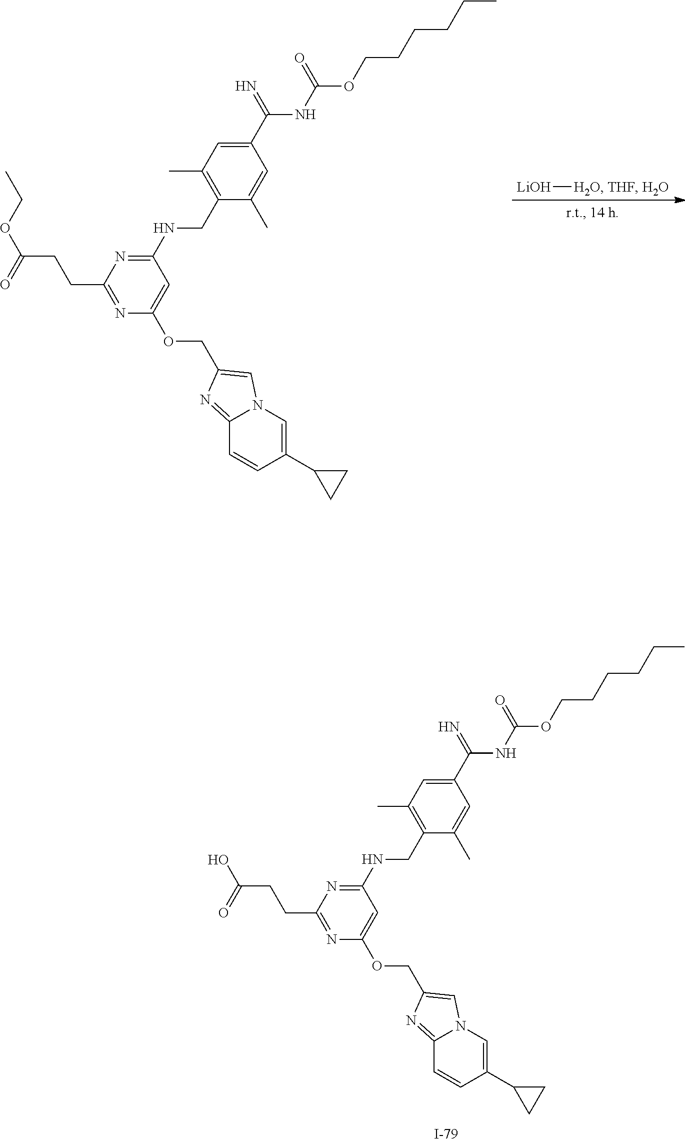

A solution of ethyl 3-(4-((6-cyclopropylimidazo[1,2-a]pyridin-2-yl)methoxy)-6-((4-(N-((hexyloxy)carbonyl)carbamimidoyl)-2,6-dimethylbenzyl)amino)pyrimidin-2-yl)propanoate (20 mg, 0.03 mmol) (synthesis reported in Example 75) and LiOH—H₂O (4.8 mg, 0.12 mmol) in THF/H₂O (2 mL/2 mL) was stirred at room temperature for 8 h. The resulting mixture was concentrated to give the crude, which was purified with Prep-HPLC to give 3-(4-((6-cyclopropylimidazo[1,2-a]pyridin-2-yl)methoxy)-6-((4-(N-((hexyloxy)carbonyl)carbamimidoyl)-2,6-dimethylbenzyl)amino)pyrimidin-2-yl)propanoic acid (5 mg, 26% yield) as a white solid. ESI-MS [M+H]+: 642.4. ¹H NMR (400 MHz, DMSO) δ9.16-9.12 (m, 2H), 8.54-8.25 (m, 2H), 7.89-7.78 (m, 1H), 7.73-7.60 (m, 2H), 7.47-7.32 (m, 1H), 7.20-7.07 (m, 1H), 7.07-6.88 (m, 1H), 5.73-5.57 (m, 1H), 5.33 (s, 2H), 4.49 (s, 2H), 4.11-3.92 (m, 2H), 2.96-2.80 (m, 2H), 2.74-2.63 (m, 2H), 2.38 (s, 6H), 1.99-1.89 (m, 1H), 1.66-1.51 (m, 2H), 1.39-1.21 (m, 6H), 0.98-0.81 (m, 5H), 0.77-0.57 (m, 2H).

### Example 80

### Synthesis of 3-(4-((6-cyclopropylimidazo[1,2-a]pyridin-2-yl)methoxy)-6-((4-(N-(ethoxycarbonyl)carbamimidoyl)-2,6-dimethylbenzyl)amino)pyrimidin-2-yl)propanoic acid (I-80)

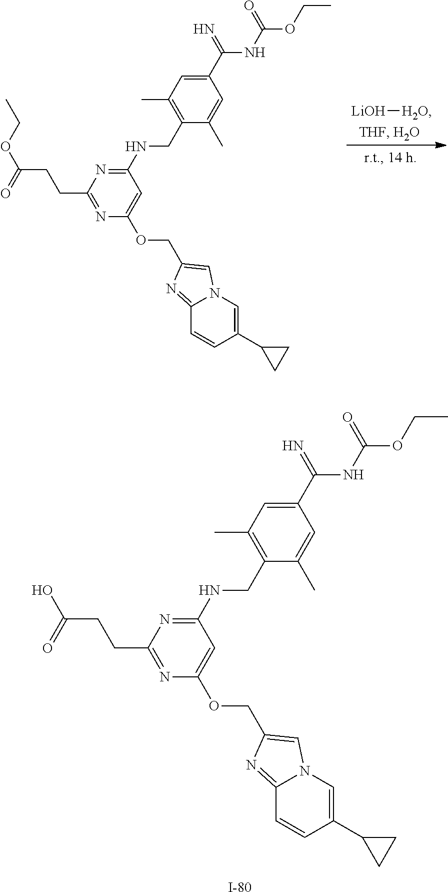

A solution of ethyl 3-(4-((6-cyclopropylimidazo[1,2-a]pyridin-2-yl)methoxy)-6-((4-(N-(ethoxycarbonyl)carbamimidoyl)-2,6-dimethylbenzyl)amino)pyrimidin-2-yl)propanoate (20 mg, 0.03 mmol) (synthesis reported in Example 77) and LiOH—H₂O (5.2 mg, 0.13 mmol) in THF/H₂O (2 mL/2 mL) was stirred at room temperature for 8 h. The resulting mixture was concentrated in vacuo to give the crude, which was purified with Prep-HPLC to give 3-(4-((6-cyclopropylimidazo[1,2-a]pyridin-2-yl)methoxy)-6-((4-(N-(ethoxycarbonyl)carbamimidoyl)-2,6-dimethylbenzyl)amino)pyrimidin-2-yl)propanoic acid (5 mg, 26% yield) as a white solid. ESI-MS [M+H]⁺: 586.3. ¹H NMR (400 MHz, DMSO) δ9.15-8.92 (m, 2H), 8.32 (s, 1H), 7.81 (s, 1H), 7.66 (s, 2H), 7.40 (d, J=9.3 Hz, 1H), 7.13-7.09 (m, 1H), 6.99 (d, J=9.3 Hz, 1H), 5.67 (s, 1H), 5.33 (s, 2H), 4.49 (s, 2H), 4.08-4.02 (m, 2H), 2.89 (t, J=6.5 Hz, 2H), 2.68 (t, J=6.6 Hz, 2H), 2.38 (s, 6H), 1.97-1.88 (m, 1H), 1.22 (t, J=7.1 Hz, 3H), 0.94-0.90 (m, 2H), 0.70-0.58 (m, 2H).

### Example 81

### Synthesis of ethyl 4-((6-cyclopropylimidazo[1,2-a]pyridin-2-yl)methoxy)-6-((4-(N-hydroxycarbamimidoyl)-2,6-dimethylbenzyl)amino)pyrimidine-2-carboxylate (I-81)

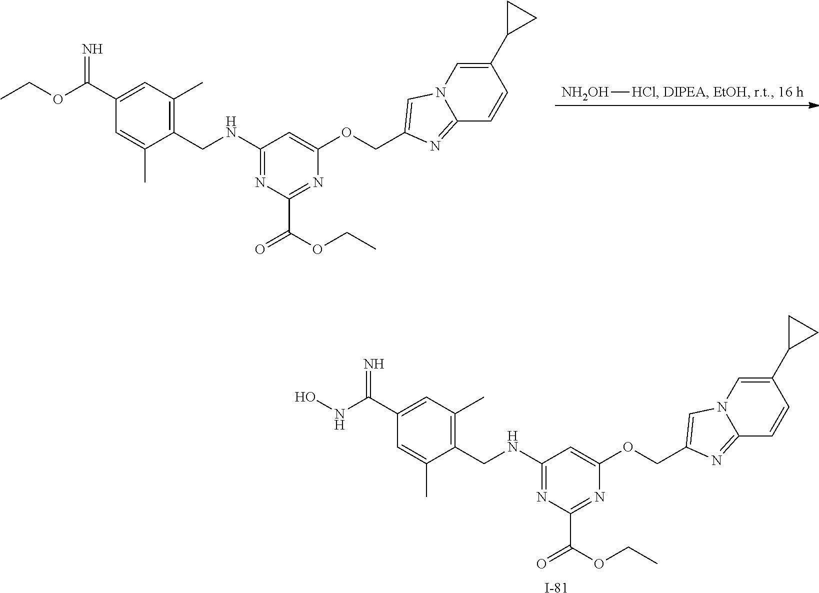

A mixture of ethyl 4-((6-cyclopropylimidazo[1,2-a]pyridin-2-yl)methoxy)-6-(4-(ethoxy(imino)methyl)-2,6-dimethylbenzylamino)pyrimidine-2-carboxylate (100 mg, crude) (synthesis reported in Example 82), NH₂OH—HCl (30 mg, 0.4 mmol) and DIPEA (0.1 mL, 0.6 mmol) in EtOH (10 mL) was stirred at room temperature for 16 h. The reaction was concentrated and purified by Prep-HPLC to afford the product ethyl 4-((6-cyclopropylimidazo[1,2-a]pyridin-2-yl)methoxy)-6-(4-(N-hydroxycarbamimidoyl)-2,6-dimethylbenzylamino)pyrimidine-2-carboxylate (30.6 mg, yield: 24% over 2 steps) as a white solid. ESI-MS [M+H]⁺: 530.3. ¹H NMR (400 MHz, DMSO) δ9.52 (s, 1H), 8.33 (s, 1H), 7.85 (s, 1H), 7.55 (s, 1H), 7.41 (d, J=9.3 Hz, 1H), 7.34 (s, 2H), 7.00 (dd, J=9.4, 1.7 Hz, 1H), 5.99 (s, 1H), 5.71 (s, 2H), 5.37 (s, 2H), 4.47 (s, 2H), 4.32 (q, J=7.1 Hz, 2H), 2.33 (s, 6H), 1.93-1.90 (m, 1H), 1.32 (t, J=7.1 Hz, 3H), 1.03-0.80 (m, 2H), 0.80-0.54 (m, 2H).

### Example 82

### Synthesis of ethyl 4-((4-carbamimidoyl-2,6-dimethylbenzyl)amino)-6-((6-cyclopropylimidazo[1,2-a]pyridin-2-yl)methoxy)pyrimidine-2-carboxylate (I-82)

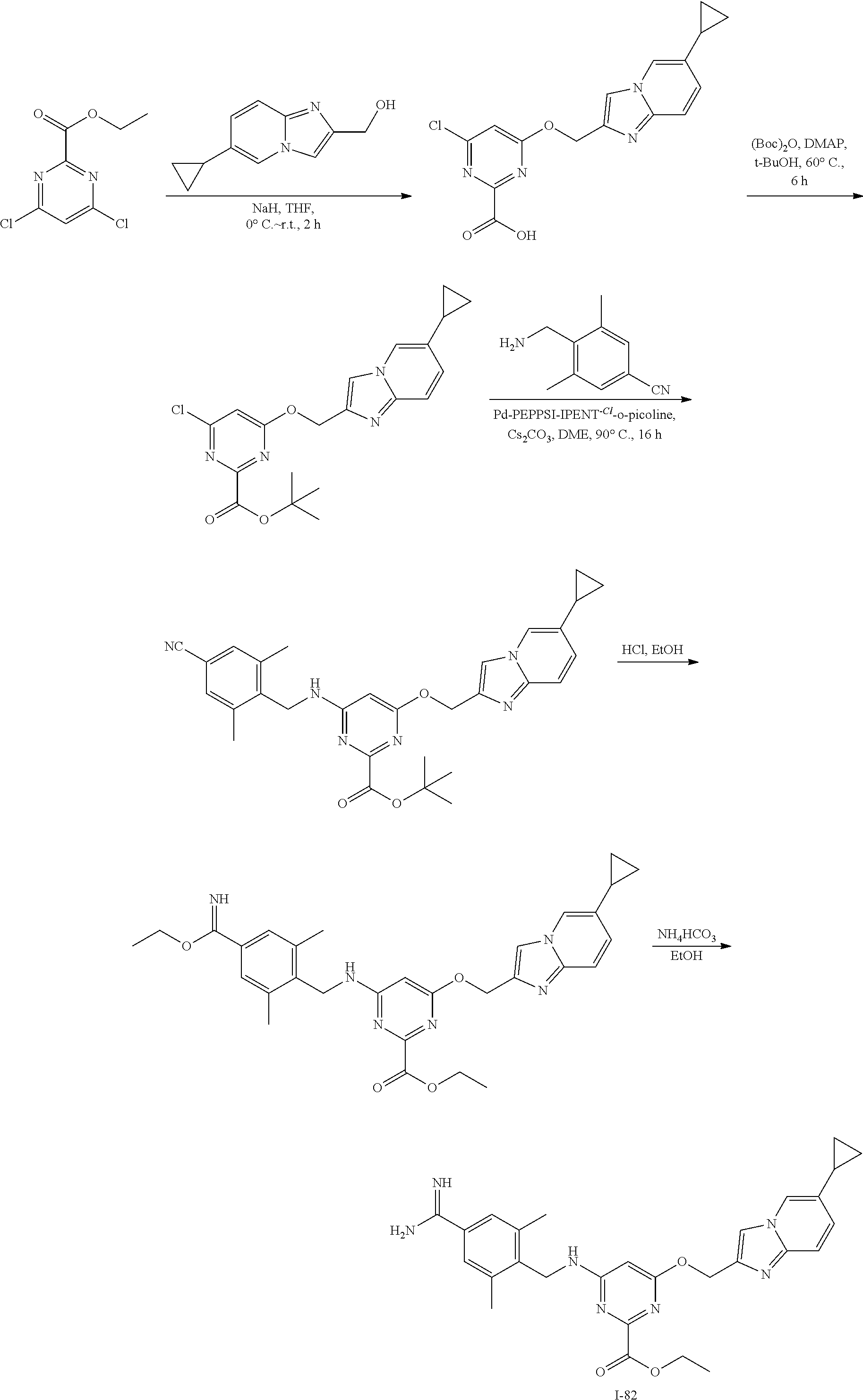

Synthesis of 4-chloro-6-((6-cyclopropylimidazo[1,2-a]pyridin-2-yl)methoxy)pyrimidine-2-carboxylic acid. To a solution of (6-cyclopropylimidazo[1,2-a]pyridin-2-yl)methanol (470 mg, 2.5 mmol) in dry THF (10 mL) was added NaH (230 mg, 5.73 mmol, 60% dispersion in mineral oil) at 0° C. and the resulting mixture was stirred at the same temperature for 0.5 h. A solution of ethyl 4,6-dichloropyrimidine-2-carboxylate (600 mg, 2.70 mmol) in THF (5 mL) was added and stirred at room temperature for 2 h. Water (30 mL) was added and the pH of the mixture was adjusted to 5 by HCl (1M aq.). The mixture was then extracted with CHCl₃/i-PrOH (3/1, 50 mL×5). The combined organic layers were dried over Na₂SO₄, filtered and concentrated in vacuo to give 4-chloro-6-((6-cyclopropylimidazo[1,2-a]pyridin-2-yl)methoxy)pyrimidine-2-carboxylic acid as a yellow solid (600 mg, crude), which was used into next step without further purification. ESI-MS [M+H]⁺: 345.1.

Synthesis of tert-butyl 4-chloro-6-((6-cyclopropylimidazo[1,2-a]pyridin-2-yl)methoxy)pyrimidine-2-carboxylate. A mixture of 4-chloro-6-((6-cyclopropylimidazo[1,2-a]pyridin-2-yl)methoxy)pyrimidine-2-carboxylic acid (600 mg, crude), (Boc)2O (2.0 g, 9.0 mmol) and DMAP (73.2 mg, 0.6 mmol) in t-BuOH (20 mL) was stirred at 60° C. for 6 h. The reaction was cooled to room temperature and concentrated in vacuo to give the crude, which was purified with silica gel chromatograph, eluting with a gradient of 0-50% EtOAc in PE to afford tert-butyl 4-chloro-6-((6-cyclopropylimidazo[1,2-a]pyridin-2-yl)methoxy)pyrimidine-2-carboxylate as yellow oil (500 mg, 50% over 2 steps). ESI-MS [M+H]⁺: 401.1.

Synthesis of tert-butyl 4-(4-cyano-2,6-dimethylbenzylamino)-6-((6-cyclopropylimidazo[1,2-a]pyridin-2-yl)methoxy)pyrimidine-2-carboxylate. A mixture of tert-butyl 4-chloro-6-((6-cyclopropylimidazo[1,2-a]pyridin-2-yl)methoxy)pyrimidine-2-carboxylate (500 mg, 1.25 mmol), 4-(aminomethyl)-3,5-dimethylbenzonitrile hydrochloride (300 mg, 1.50 mmol), Pd-PEPPSI-IPENT-Cl-o-picoline (100 mg, 0.125 mmol) and Cs₂CO₃ (1.0 g, 3.125 mmol) in DME (15 mL) was stirred at 95° C. 16 h under N₂. The mixture was cooled to room temperature and concentrated in vacuo to give the residue, which was purified with silica gel chromatograph, eluting with a gradient of 0-10% MeOH in DCM to afford tert-butyl 4-((4-cyano-2,6-dimethylbenzyl)amino)-6-((6-cyclopropylimidazo[1,2-a]pyridin-2-yl)methoxy)pyrimidine-2-carboxylate as yellow oil (250 mg, yield: 38%). ESI-MS [M+H]+: 525.3.

Synthesis of ethyl 4-((6-cyclopropylimidazo[1,2-a]pyridin-2-yl)methoxy)-6-(4-(ethoxy(imino)methyl)-2,6-dimethylbenzylamino)pyrimidine-2-carboxylate. A solution of tert-butyl 4-(4-cyano-2,6-dimethylbenzylamino)-6-((6-cyclopropylimidazo[1,2-a]pyridin-2-yl)methoxy)pyrimidine-2-carboxylate (250 mg, 0.48 mmol) in EtOH (15 mL) was bubbled dry HCl gas at room temperature for 2 h. The reaction was concentrated in vacuo to afford ethyl 4-((6-cyclopropylimidazo[1,2-a]pyridin-2-yl)methoxy)-6-(4-(ethoxy(imino)methyl)-2,6-dimethylbenzylamino)pyrimidine-2-carboxylate (200 mg, crude) as a yellow solid, which was used into the next step without further purification. ESI-MS [M+H]+: 543.1.

Synthesis of ethyl 4-(4-carbamimidoyl-2,6-dimethylbenzylamino)-6-((6-cyclopropylimidazo[1,2-a]pyridin-2-yl)methoxy)pyrimidine-2-carboxylate. A mixture of ethyl 4-((6-cyclopropylimidazo[1,2-a]pyridin-2-yl)methoxy)-6-(4-(ethoxy(imino)methyl)-2,6-dimethylbenzylamino)pyrimidine-2-carboxylate (100 mg, crude) and NH₄HCO₃ (160 mg, 2.0 mmol) in EtOH (10 mL) was stirred at room temperature for 16 h. The reaction was concentrated and purified by Prep-HPLC to afford the product ethyl 4-(4-carbamimidoyl-2,6-dimethylbenzylamino)-6-((6-cyclopropylimidazo[1,2-a]pyridin-2-yl)methoxy)pyrimidine-2-carboxylate as formate salt (33.9 mg, yield: 25% over 2 steps) as a white solid. ESI-MS [M+H]⁺: 514.3. ¹H NMR (400 MHz, DMSO) δ 10.91 (s, 2H), 8.92 (s, 2H), 8.42 (s, 1H), 8.33 (s, 1H), 7.85 (s, 1H), 7.71 (s, 1H), 7.48 (s, 2H), 7.41 (d, J=9.3 Hz, 1H), 7.00 (dd, J=9.4, 1.7 Hz, 1H), 6.00 (s, 1H), 5.37 (s, 2H), 4.54 (s, 2H), 4.32 (q, J=7.1 Hz, 2H), 2.45 (s, 6H), 2.00-1.86 (m, 1H), 1.33 (t, J=7.1 Hz, 3H), 0.96-0.85 (m, 2H), 0.68 (dd, J=5.0, 1.8 Hz, 2H).

### Example 83

### Synthesis of ethyl ((4-(((6-((6-cyclopropylimidazo[1,2-a]pyridin-2-yl)methoxy)pyrimidin-4-yl)amino)methyl)-3,5-dimethylphenyl)(imino)methyl)carbamate (I-83)

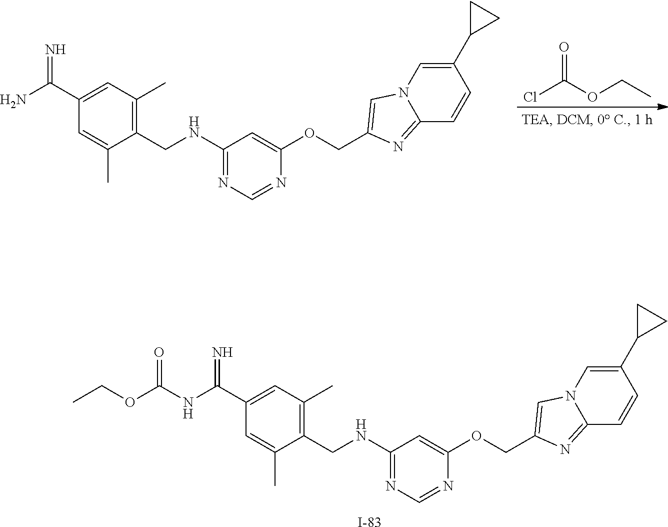

To a mixture of 4-(((6-((6-cyclopropylimidazo[1,2-a]pyridin-2-yl)methoxy)pyrimidin-4-yl)amino)methyl)-3,5-dimethylbenzimidamide (125 mg, 0.28 mmol) (synthesis reported in Example 72) and TEA (141 mg, 1.4 mmol) in DCM (10 mL) was added hexyl carbonochloridate (61 mg, 0.56 mmol) and the resulting mixture was stirred at 0° C. for 1 h. The mixture was concentrated and purified by Prep-HPLC to give ethyl ((4-(((6-((6-cyclopropylimidazo[1,2-a]pyridin-2-yl)methoxy)pyrimidin-4-yl)amino)methyl)-3,5-dimethylphenyl)(imino)methyl)carbamate (25 mg, yield: 17%) as a white solid. ESI-MS [M+H]⁺: 514.3. ¹H NMR (400 MHz, DMSO) δ9.14 (s, 1H), 8.93 (s, 1H), 8.33 (s, 1H), 8.26 (s, 1H), 7.81 (s, 1H), 7.68 (s, 2H), 7.40 (d, J=9.3 Hz, 1H), 7.22 (t, J=4.6 Hz, 1H), 6.96-7.03 (m, 1H), 5.86 (s, 1H), 5.35 (s, 2H), 4.48 (s, 2H), 4.01-4.09 (m, 2H), 2.38 (s, 6H), 1.88-1.97 (m, 1H), 1.22 (t, J=7.1 Hz, 3H), 0.96-0.88 (m, 2H), 0.70-0.64 (m, 2H).

### Example 84

### Synthesis of 4-((6-cyclopropylimidazo[1,2-a]pyridin-2-yl)methoxy)-6-((4-(N-hydroxycarbamimidoyl)-2,6-dimethylbenzyl)amino)pyrimidine-2-carboxylic acid (I-84)

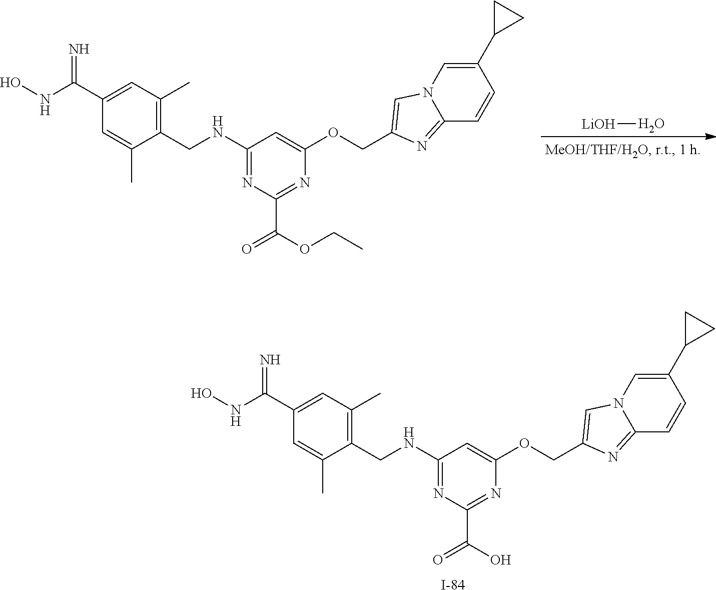

A mixture of ethyl 4-((6-cyclopropylimidazo[1,2-a]pyridin-2-yl)methoxy)-6-((4-(N-hydroxycarbamimidoyl)-2,6-dimethylbenzyl)amino)pyrimidine-2-carboxylate (15 mg, 0.03 mmol) (synthesis reported in Example 81) and LiOH—H₂O (2.5 mg, 0.06 mmol) in MeOH/THF/H₂O (1 mL/1 mL/1 mL) was stirred at room temperature for 1 h. The mixture was concentrated to give the crude, which was purified by Prep-HPLC to give 4-((6-cyclopropylimidazo[1,2-a]pyridin-2-yl)methoxy)-6-((4-(N-hydroxycarbamimidoyl)-2,6-dimethylbenzyl)amino)pyrimidine-2-carboxylic acid (4 mg, yield: 27%) as a white solid. ESI-MS [M+H]⁺: 502.3. ¹H NMR (400 MHz, DMSO) δ9.53 (s, 1H), 8.33 (s, 1H), 7.84 (s, 1H), 7.44-7.40 (m, 2H), 7.40-7.36 (m, 2H), 7.00 (dd, J=9.4, 1.7 Hz, 1H), 5.95 (s, 1H), 5.72 (s, 2H), 5.39 (s, 2H), 4.47 (s, 2H), 2.34 (s, 6H), 2.01-1.83 (m, 1H), 1.00-0.84 (m, 2H), 0.76-0.57 (m, 2H).

### Example 85

### Synthesis of 4-((4-carbamimidoyl-2,6-dimethylbenzyl)amino)-6-((6-cyclopropylimidazo[1,2-a]pyridin-2-yl)methoxy)pyrimidine-2-carboxylic acid (I-85)

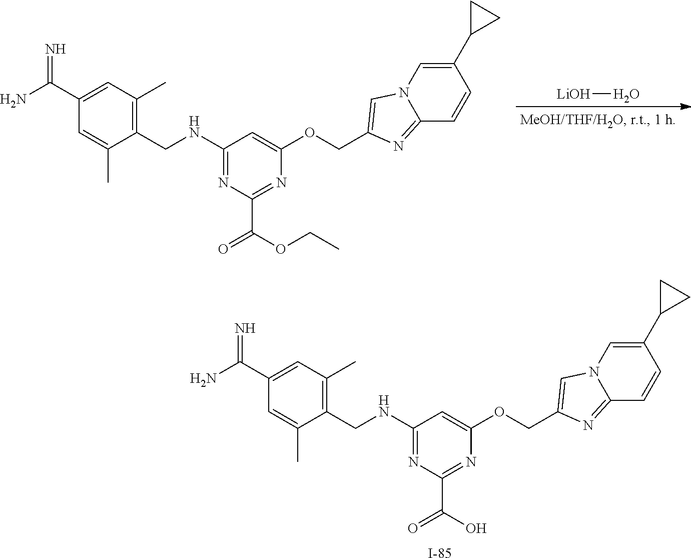

A mixture of ethyl 4-((4-carbamimidoyl-2,6-dimethylbenzyl)amino)-6-((6-cyclopropylimidazo[1,2-a]pyridin-2-yl)methoxy)pyrimidine-2-carboxylate (15 mg, 0.03 mmol) (synthesis reported in Example 82) and LiOH—H₂O (2.4 mg, 0.06 mmol) in MeOH/THF/H₂O (1 mL/1 mL/1 mL) was stirred at room temperature for 1 h. The mixture was concentrated to give the crude, which was purified by Prep-HPLC to give 4-((4-carbamimidoyl-2,6-dimethylbenzyl)amino)-6-((6-cyclopropylimidazo[1,2-a]pyridin-2-yl)methoxy)pyrimidine-2-carboxylic acid as formate salt (7.5 mg, yield: 47%) as a white solid. ESI-MS [M+H]⁺: 486.2. ¹H NMR (400 MHz, DMSO) δ10.58 (s, 2H), 9.07 (s, 2H), 8.32 (s, 1H), 8.27 (s, 1H), 7.84 (s, 1H), 7.49 (s, 2H), 7.40 (d, J=9.3 Hz, 1H), 7.25 (s, 1H), 6.99 (d, J=9.3 Hz, 1H), 5.78 (s, 1H), 5.34 (s, 2H), 4.49 (s, 2H), 2.44 (s, 6H), 1.96-1.89 (m, 1H), 1.02-0.82 (m, 2H), 0.68-0.67 (m, 2H).

### Example 86

### Synthesis of ethyl 4-((6-cyclopropylimidazo[1,2-a]pyridin-2-yl)methoxy)-6-(4-(N-(ethoxycarbonyl)carbamimidoyl)-2,6-dimethylbenzylamino)pyrimidine-2-carboxylate (I-86)

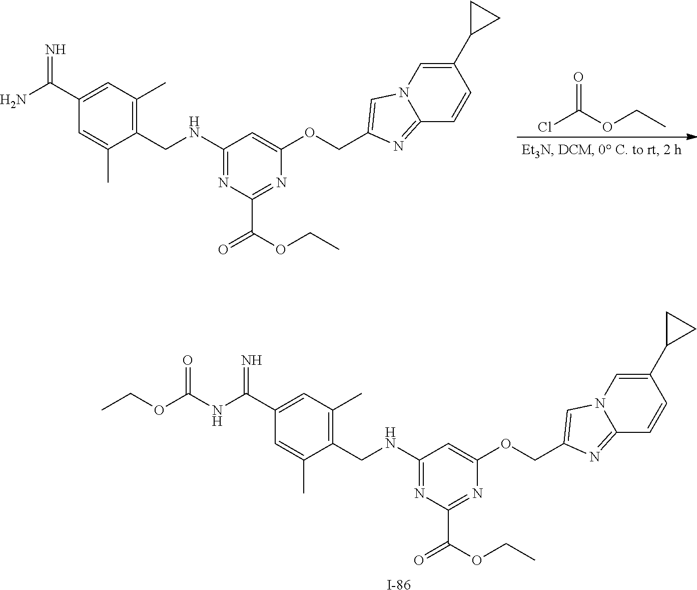

To a mixture of ethyl 4-((4-carbamimidoyl-2,6-dimethylbenzyl)amino)-6-((6-cyclopropylimidazo[1,2-a]pyridin-2-yl)methoxy)pyrimidine-2-carboxylate (100 mg, 0.19 mmol) (synthesis reported in Example 82) and Et₃N (58 mg, 0.57 mmol) in DCM (5 mL) was added ethyl carbonochloridate (41 mg, 0.38 mmol) at 0° C. and the reaction mixture was stirred at room temperature for 2 h. The reaction was quenched with water (30 mL) and extracted with EtOAc (30 mL×3). The combined organic layers were washed with brine (30 mL), dried over Na₂SO₄, concentrated to give the crude, which was purified by Prep-TLC (eluent: DCM/MeOH=10/1) to give ethyl 4-((6-cyclopropylimidazo[1,2-a]pyridin-2-yl)methoxy)-6-(4-(N-(ethoxycarbonyl)carbamimidoyl)-2,6-dimethylbenzylamino)pyrimidine-2-carboxylate (35 mg, yield: 32%) as a light yellow solid. ESI-MS [M+H]+: 586.1. 1H NMR (400 MHz, DMSO) δ9.20-8.80 (m, 2H), 8.33 (s, 1H), 7.85 (s, 1H), 7.67-7.62 (m, 3H), 7.41 (d, J=9.3 Hz, 1H), 7.00 (dd, J=9.4, 1.8 Hz, 1H), 6.00 (s, 1H), 5.37 (s, 2H), 4.52 (s, 2H), 4.32 (q, J=7.0 Hz, 2H), 4.08-4.02 (m, 2H), 2.41 (s, 6H), 2.08-1.74 (m, 1H), 1.33 (t, J=7.0 Hz, 3H), 1.22 (t, J=7.1 Hz, 3H), 1.01-0.85 (m, 2H), 0.77-0.66 (m, 2H).

### Example 87

Synthesis of ethyl 4-((6-cyclopropylimidazo[1,2-a]pyridin-2-yl)methoxy)-6-(4-(N-(hexyloxycarbonyl)carbamimidoyl)-2,6-dimethylbenzylamino)pyrimidine-2-carboxylate (I-87)

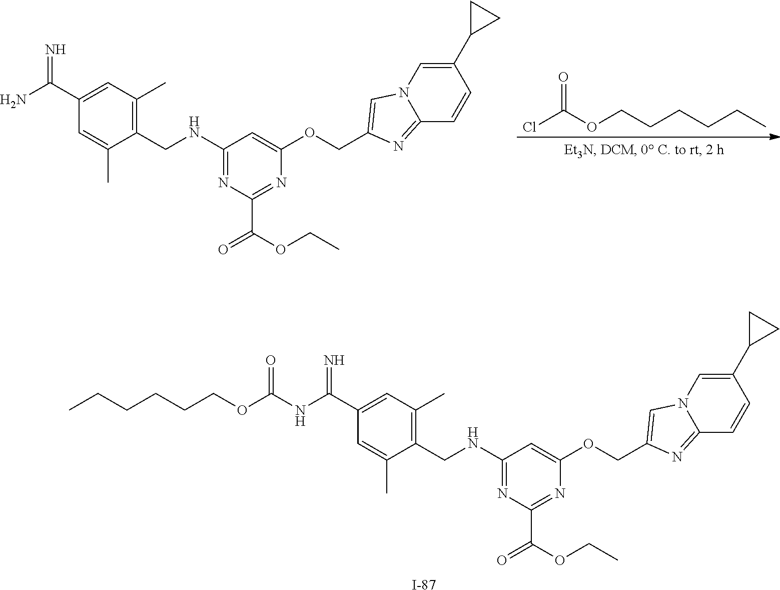

A mixture of ethyl 4-((4-carbamimidoyl-2,6-dimethylbenzyl)amino)-6-((6-cyclopropylimidazo[1,2-a]pyridin-2-yl)methoxy)pyrimidine-2-carboxylate (100 mg, 0.19 mmol) (synthesis reported in Example 82) and Et₃N (58 mg, 0.57 mmol) in DCM (5 mL) was added hexyl carbonochloridate (62 mg, 0.38 mmol) at 0° C. and the mixture was stirred at room temperature for 2 h. The reaction was quenched with water (30 mL) and extracted with EtOAc (30 mL×3). The combined organic layers were washed with brine (30 mL), dried over Na₂SO₄, concentrated to give the crude, which was purified by Prep-TLC (eluent: DCM/MeOH=10/1) to give ethyl 4-((6-cyclopropylimidazo[1,2-a]pyridin-2-yl)methoxy)-6-(4-(N-(hexyloxycarbonyl)carbamimidoyl)-2,6-dimethylbenzylamino)pyrimidine-2-carboxylate (36 mg, yield: 30%) as a light yellow solid. ESI-MS [M+H]⁺: 642.1. 1H NMR (400 MHz, DMSO) δ9.25-8.90 (m, 2H), 8.33 (s, 1H), 7.85 (s, 1H), 7.65 (s, 3H), 7.41 (d, J=9.5 Hz, 1H), 6.99 (d, J=9.2 Hz, 1H), 6.00 (s, 1H), 5.37 (s, 2H), 4.51 (s, 2H), 4.43-4.23 (m, 2H), 4.04-3.98 (m, 2H), 2.40 (s, 6H), 1.93 (s, 1H), 1.62-1.54 (m, 2H), 1.42-1.23 (m, 9H), 0.98-0.85 (m, 5H), 0.71-0.65 (m, 2H).

### Example 88

### Synthesis of 4-((6-cyclopropylimidazo[1,2-a]pyridin-2-yl)methoxy)-6-(4-(N-(ethoxycarbonyl)carbamimidoyl)-2,6-dimethylbenzylamino)pyrimidine-2-carboxylic acid (I-88)

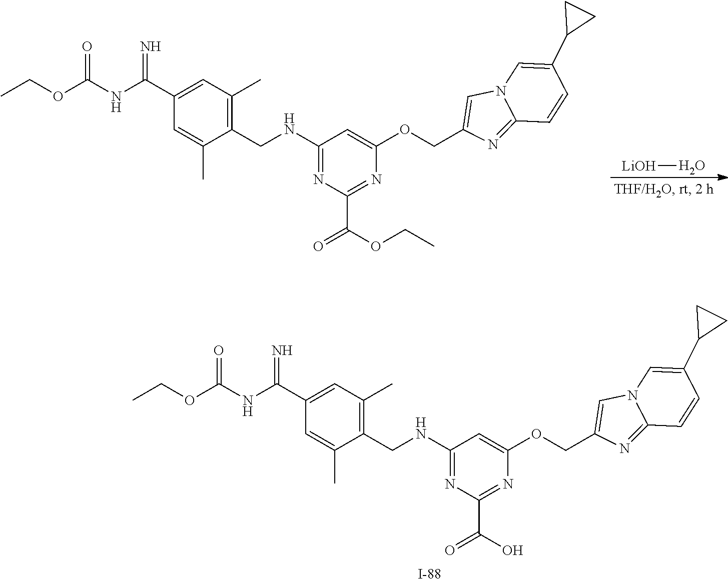

A mixture of ethyl 4-((6-cyclopropylimidazo[1,2-a]pyridin-2-yl)methoxy)-6-(4-(N-(ethoxycarbonyl)carbamimidoyl)-2,6-dimethylbenzylamino)pyrimidine-2-carboxylate (15 mg, 0.026 mmol) (synthesis reported in Example 86) and LiOH—H₂O (5.5 mg, 0.13 mmol) in THF/water (2 mL/2 mL) was stirred at room temperature for 2 h. The mixture was concentrated and purified by Prep-HPLC to give 4-((6-cyclopropylimidazo[1,2-a]pyridin-2-yl)methoxy)-6-(4-(N-(ethoxycarbonyl)carbamimidoyl)-2,6-dimethylbenzylamino)pyrimidine-2-carboxylic acid (5.0 mg, yield: 36%) as a white solid. ESI-MS [M+H]⁺: 558.1. ¹H NMR (400 MHz, DMSO) δ9.25-8.90 (m, 2H), 8.33 (s, 1H), 7.85 (s, 1H), 7.67 (s, 2H), 7.43-7.39 (m, 2H), 7.00 (d, J=8.8 Hz, 1H), 5.90 (s, 1H), 5.38 (s, 2H), 4.49 (s, 2H), 4.08-4.03 (m, 2H), 2.39 (s, 6H), 1.96-1.89 (m, 1H), 1.22 (t, J=7.0 Hz, 3H), 0.93-0.91 (m, 2H), 0.69-0.67 (m, 2H).

### Example 89

### Synthesis of 4-((6-cyclopropylimidazo[1,2-a]pyridin-2-yl)methoxy)-6-(4-(N-(hexyloxycarbonyl)carbamimidoyl)-2,6-dimethylbenzylamino)pyrimidine-2-carboxylic acid (I-89)

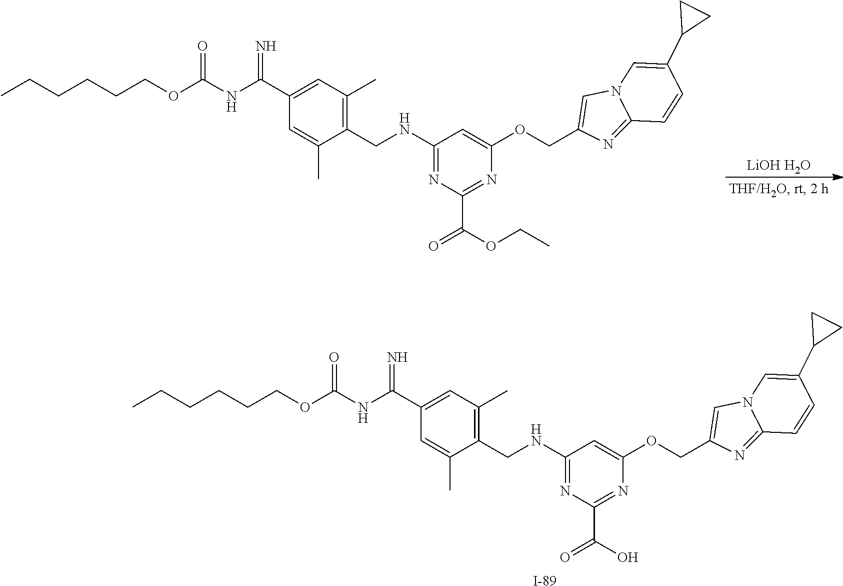

A solution of ethyl 4-((6-cyclopropylimidazo[1,2-a]pyridin-2-yl)methoxy)-6-(4-(N-(hexyloxycarbonyl)carbamimidoyl)-2,6-dimethylbenzylamino)pyrimidine-2-carboxylate (20 mg, 0.031 mmol) (synthesis reported in Example 87) and LiOH—H₂O (6.6 mg, 0.16 mmol) in THF/water (2 mL/2 mL) was stirred at rt for 2 h. The mixture was concentrated and purified by Prep-HPLC to give 4-((6-cyclopropylimidazo[1,2-a]pyridin-2-yl)methoxy)-6-(4-(N-(hexyloxycarbonyl)carbamimidoyl)-2,6-dimethylbenzylamino)pyrimidine-2-carboxylic acid (6 mg, yield: 32%) as a white solid. ESI-MS [M+H]⁺: 615.1. ¹H NMR (400 MHz, DMSO) δ9.26-8.91 (m, 2H), 8.33-8.27 (m, 2H), 7.88 (s, 1H), 7.66-7.57 (m, 2H), 7.42 (d, J=9.3 Hz, 1H), 6.99 (d, J=9.3 Hz, 1H), 5.84 (s, 1H), 5.38 (s, 2H), 4.39 (s, 2H), 3.99 (t, J=6.6 Hz, 2H), 2.39 (s, 6H), 1.93 (s, 1H), 1.68-1.54 (m, 2H), 1.29 (s, 6H), 1.01-0.81 (m, 5H), 0.68 (d, J=5.3 Hz, 2H).

### Example 90

Synthesis of ((4-(((6-((6-cyclopropylimidazo[1,2-a]pyridin-2-yl)methoxy)pyrimidin-4-ylamino)methyl)-3,5-dimethylbenzimidamido)oxy)methyl acetate (I-90)

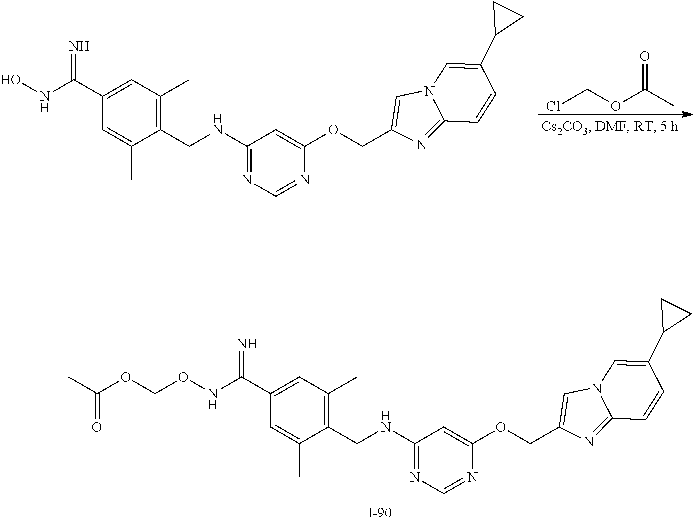

To a mixture of 4-(((6-((6-cyclopropylimidazo[1,2-a]pyridin-2-yl)methoxy)pyrimidin-4-yl)amino)methyl)-N-hydroxy-3,5-dimethylbenzimidamide (300 mg, 0.66 mmol) and Cs₂CO₃ (430 mg, 1.32 mmol) in DMF (10 mL) was added chloromethyl acetate (86 mg, 0.80 mmol). The mixture was stirred at room temperature for 5 h. Water (50 mL) was added and the mixture was extracted with EtOAc (50 mL×3). The combined organic layers were washed with brine (50 mL), dried over Na₂SO₄, concentrated to give the crude, which was purified by Prep-HPLC to give ((4-(((6-((6-cyclopropylimidazo[1,2-a]pyridin-2-yl)methoxy)pyrimidin-4-yl)amino)methyl)-3,5-dimethylbenzimidamido)oxy)methyl acetate (18 mg, yield: 5%) as a white solid. ESI-MS [M+H]⁺: 530.2. ¹H NMR (400 MHz, DMSO) δ8.33 (s, 1H), 8.26 (s, 1H), 7.81 (s, 1H), 7.40 (d, J=9.3 Hz, 1H), 7.35 (s, 2H), 7.17 (t, J=4.5 Hz, 1H), 6.99 (dd, J=9.4, 1.7 Hz, 1H), 6.21 (s, 2H), 5.85 (s, 1H), 5.60 (s, 2H), 5.35 (s, 2H), 4.45 (s, 2H), 2.34 (s, 6H), 2.05 (s, 3H), 1.96-1.90 (m, 1H), 0.95-0.86 (m, 2H), 0.72-0.64 (m, 2H).

### Example 91

### Synthesis of 4-(((6-((6-cyclopropylimidazo[1,2-a]pyridin-2-yl)methoxy)-2-(2H-tetrazol-5-yl)pyrimidin-4-yl)amino)methyl)-3,5-dimethylbenzimidamide (I-91)

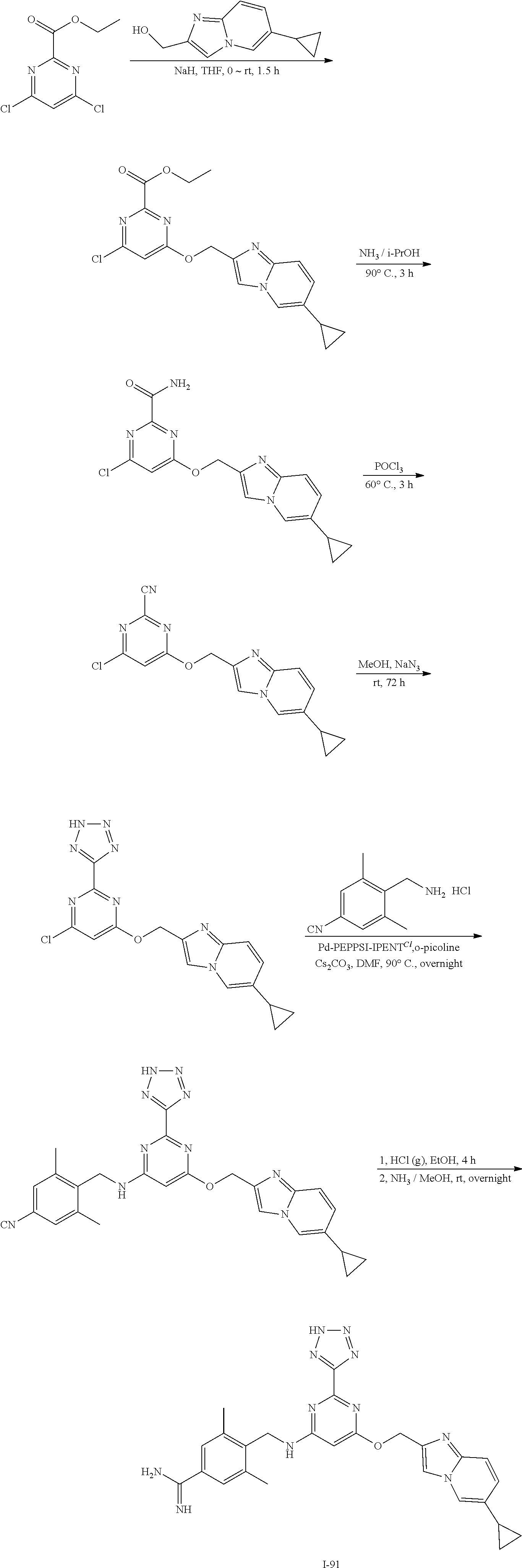

Synthesis of ethyl 4-chloro-6-((6-cyclopropylimidazo[1,2-a]pyridin-2-yl)methoxy)pyrimidine-2-carboxylate. To a solution of (6-cyclopropylimidazo[1,2-a]pyridin-2-yl)methanol (1.09 g, 5.8 mmol) in THF (20 mL) was added NaH (300 mg, 7.5 mmol, 60% in mineral oil) slowly at 0° C. The mixture was stirred at room temperature for 1 h. Then a solution of ethyl 4,6-dichloropyrimidine-2-carboxylate (1.1 g, 5.0 mmol) in THF (5 mL) was added and the mixture was stirred at room temperature for 0.5 h. The mixture was quenched with NH₄Cl (sat. aq., 50 mL) and extracted with EtOAc (50 mL×3). The combined organic layers were washed with brine (50 mL), dried over Na₂SO₄, concentrated to give the crude, which was purified by column chromatography (eluent: DCM/MeOH=0-5%) to give ethyl 4-chloro-6-((6-cyclopropylimidazo[1,2-a]pyridin-2-yl)methoxy)pyrimidine-2-carboxylate (1.2 g, yield: 65%) as a white solid. ESI-MS [M+H]⁺: 373.0.

Synthesis of 4-chloro-6-((6-cyclopropylimidazo[1,2-a]pyridin-2-yl)methoxy)pyrimidine-2-carboxamide. A solution of ethyl 4-chloro-6-((6-cyclopropylimidazo[1,2-a]pyridin-2-yl)methoxy)pyrimidine-2-carboxylate (1.2 g, 3.2 mmol) in NH₃/iPrOH (8 mL) was stirred in a sealed tube at 90° C. for 3 h. After cooling to room temperature, the mixture was concentrated to give 4-chloro-6-((6-cyclopropylimidazo[1,2-a]pyridin-2-yl)methoxy)pyrimidine-2-carboxamide (1.1 g, yield: quant) as a white solid which was used to the next step without further purification. ESI-MS [M+H]⁺: 343.9.

Synthesis of 4-chloro-6-((6-cyclopropylimidazo[1,2-a]pyridin-2-yl)methoxy)pyrimidine-2-carbonitrile. A mixture of 4-chloro-6-((6-cyclopropylimidazo[1,2-a]pyridin-2-yl)methoxy)pyrimidine-2-carboxamide (1.1 g, 3.2 mmol) in POCl₃ (10 mL) was stirred at 60° C. for 3 h. After cooling to room temperature, the mixture was concentrated to give a residue, which was purified by column chromatography (eluent: DCM/MeOH=0-5%) to give 4-chloro-6-((6-cyclopropylimidazo[1,2-a]pyridin-2-yl)methoxy)pyrimidine-2-carbonitrile (1.0 g, yield: 96%) as a whilte solid. ESI-MS [M+H]+: 326.0.

Synthesis of 2-(((6-chloro-2-(2H-tetrazol-5-yl)pyrimidin-4-yl)oxy)methyl)-6-cyclopropylimidazo[1,2-a]pyridine. A solution of 4-chloro-6-((6-cyclopropylimidazo[1,2-a]pyridin-2-yl)methoxy)pyrimidine-2-carbonitrile (1.0 g, 3.07 mmol) and sodium azide (598 mg, 9.2 mmol) in MeOH (20 mL) was stirred at room temperature for 3 days. The mixture was quenched with water (100 mL) and extracted with EtOAc (50 mL×5). The combined organic layers were washed with brine (100 mL), dried over Na₂SO₄, concentrated to give the crude, which was purified by column chromatography (eluent: DCM/MeOH=0-20%) to give 2-(((6-chloro-2-(2H-tetrazol-5-yl)pyrimidin-4-yl)oxy)methyl)-6-cyclopropylimidazo[1,2-a]pyridine (370 mg, yield: 33%) as a yellow solid. ESI-MS [M+H]+: 369.0.

Synthesis of 6-((6-cyclopropylimidazo[1,2-a]pyridin-2-yl)methoxy)-N-(4-isocyano-2,6-dimethylbenzyl)-2-(2H-tetrazol-5-yl)pyrimidin-4-amine. A solution of 2-(((6-chloro-2-(2H-tetrazol-5-yl)pyrimidin-4-yl)oxy)methyl)-6-cyclopropylimidazo[1,2-a]pyridine (333 mg, 0.90 mmol), (4-isocyano-2,6-dimethylphenyl)methanamine hydrochloride (265 mg, 1.35 mmol), Pd-PEPPSI-IPENTCl, o-picoline (76 mg, 0.09 mmol) and CSCO₃ (733 mg, 2.25 mmol) in DMF (6 mL) was stirred at 90° C. under N₂ for 18 h. After cooling to room temperature, the reaction was quenched with water (100 mL) and extracted with CH₃Cl/i-PrOH (3/1, 50 mL×5). The combined organic layers were washed with brine (50 mL), dried over anhydrous Na₂SO₄, and concentrated to give a residue, which was purified by Pre-TLC (eluent: DCM/MeOH=4:1) to give 6-((6-cyclopropylimidazo[1,2-a]pyridin-2-yl)methoxy)-N-(4-isocyano-2,6-dimethylbenzyl)-2-(2H-tetrazol-5-yl)pyrimidin-4-amine (100 mg, yield: 23%) as a yellow solid. ESI-MS [M+H]+: 493.0.

Synthesis of 4-(((6-((6-cyclopropylimidazo[1,2-a]pyridin-2-yl)methoxy)-2-(2H-tetrazol-5-yl)pyrimidin-4-yl)amino)methyl)-3,5-dimethylbenzimidamide. HCl gas was bubbled to a solution of 6-((6-cyclopropylimidazo[1,2-a]pyridin-2-yl)methoxy)-N-(4-isocyano-2,6-dimethylbenzyl)-2-(2H-tetrazol-5-yl)pyrimidin-4-amine (100 mg, 0.20 mmol) in ethanol (8 mL) for 4 h at room temperature. The mixture was concentrated to get a residue which was dissolved in MeOH (4 mL). To the above solution added NH₃/MeOH (4 mL, 7.0 M) at 0° C. The mixture was stirred at room temperature for 18 h. The mixture was concentrated to get a residue, which was purified by Prep-HPLC to give 4-(((6-((6-cyclopropylimidazo[1,2-a]pyridin-2-yl)methoxy)-2-(2H-tetrazol-5-yl)pyrimidin-4-yl)amino)methyl)-3,5-dimethylbenzimidamide as formate salt (12.3 mg, yield: 11%) as a white solid. ESI-MS [M+H]+: 510.2. ¹H NMR (400 MHz, DMSO) δ9.83 (s, 2H), 8.93 (s, 2H), 8.12-8.08 (m, 2H), 7.73 (s, 1H), 7.23-7.18 (m, 3H), 6.99 (s, 1H), 6.76 (dd, J=9.4, 1.5 Hz, 1H), 5.55 (s, 1H), 5.22 (s, 2H), 4.36 (s, 2H), 2.23 (s, 6H), 1.74-1.67 (m, 1H), 0.71-0.67 (m, 2H), 0.48-0.44 (m, 2H).

### Example 92 and Example 93

Synthesis of 3-(4-((4-carbamimidoyl-2,6-dimethylbenzyl)amino)-6-((6-cyclopropylimidazo[1,2-a]pyridin-2-yl)methoxy)pyrimidin-2-yl)-2,2-dimethylpropanoic acid (I-92) and ethyl 3-(4-((4-carbamimidoyl-2,6-dimethylbenzyl)amino)-6-((6-cyclopropylimidazo[1,2-a]pyridin-2-yl)methoxy)pyrimidin-2-yl)-2,2-dimethylpropanoate (I-93)

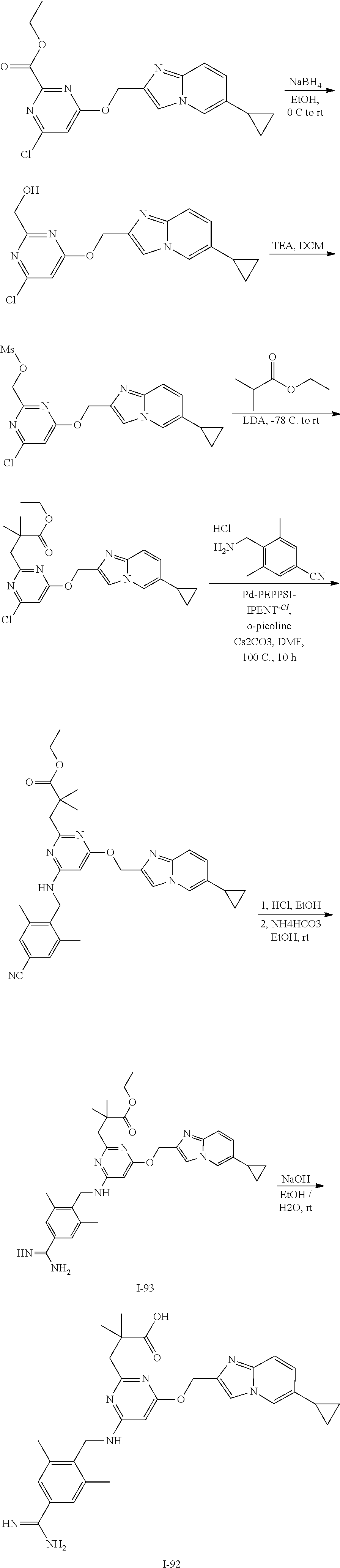

Synthesis of (4-chloro-6-((6-cyclopropylimidazo[1,2-a]pyridin-2-yl)methoxy)pyrimidin-2-yl)methanol. To a mixture of ethyl 4-chloro-6-((6-cyclopropylimidazo[1,2-a]pyridin-2-yl)methoxy)pyrimidine-2-carboxylate (920 mg, 2.5 mmol) (synthesis reported in Example 91) in EtOH (20 mL) was added NaBH₄ (285 mg, 7.5 mmol) at 0° C., the resulting mixture was stirred at room temperature for 2 h. The reaction was quenched with water (50 mL) and extracted with EtOAc (50 mL×3). The combined organic layers were washed with brine (50 mL), dried over Na₂SO₄, concentrated to give the crude, which was purified by column chromatography (eluent: EtOAc/PE=0−60%) to give (4-chloro-6-((6-cyclopropylimidazo[1,2-a]pyridin-2-yl)methoxy)pyrimidin-2-yl)methanol (450 mg, yield: 55%). ESI-MS [M+H]⁺: 331.2.

Synthesis of (4-chloro-6-((6-cyclopropylimidazo[1,2-a]pyridin-2-yl)methoxy)pyrimidin-2-yl)methyl methanesulfonate. A mixture of (4-chloro-6-((6-cyclopropylimidazo[1,2-a]pyridin-2-yl)methoxy)pyrimidin-2-yl)methanol (450 mg, 1.36 mmol), methanesulfonyl chloride (163 mg, 1.43 mmol) and TEA (481 mg, 4.75 mmol) in DCM (15 mL) was stirred at room temperature for 3 h. The reaction was quenched with water (50 mL) and extracted with EtOAc (50 mL×3). The combined organic layers were washed with brine (50 mL), dried over Na₂SO₄, concentrated to give (4-chloro-6-((6-cyclopropylimidazo[1,2-a]pyridin-2-yl)methoxy)pyrimidin-2-yl)methyl methanesulfonate (560 mg, yield: quant). ESI-MS [M−63]+: 409.1.

Synthesis of ethyl 3-(4-chloro-6-((6-cyclopropylimidazo[1,2-a]pyridin-2-yl)methoxy)pyrimidin-2-yl)-2,2-dimethylpropanoate. To a solution of ethyl isobutyrate (316 mg, 2.72 mmol) in THF (10 mL) was added LDA (1.36 mL, 2.72 mmol, 2.0 M in hexane) dropwise at −78° C. and the mixture was stirred at −78° C. for 2 h. (4-chloro-6-((6-cyclopropylimidazo[1,2-a]pyridin-2-yl)methoxy)pyrimidin-2-yl)methyl methanesulfonate (560 mg, 1.36 mmol) in THF (5 mL) was added dropwise. The resulting reaction mixture was warmed to room temperature and stirred for 2 h. Then the reaction was quenched with NH₄Cl (sat. aq., 50 mL), extracted with EtOAc (30 mL×3). The combined organic layers were washed with brine (30 mL), dried over Na₂SO₄, concentrated to give the crude, which was purified by Prep-TLC (eluent: DCM/MeOH=15/1) to give ethyl 3-(4-chloro-6-((6-cyclopropylimidazo[1,2-a]pyridin-2-yl)methoxy)pyrimidin-2-yl)-2,2-dimethylpropanoate (62 mg, yield: 11%). ESI-MS [M+H]+: 429.1.

Synthesis of ethyl 3-(4-((4-cyano-2,6-dimethylbenzyl)amino)-6-((6-cyclopropylimidazo[1,2-a]pyridin-2-yl)methoxy)pyrimidin-2-yl)-2,2-dimethylpropanoate. A mixture of ethyl 3-(4-chloro-6-((6-cyclopropylimidazo[1,2-a]pyridin-2-yl)methoxy)pyrimidin-2-yl)-2,2-dimethylpropanoate (62 mg, 0.145 mmol), 4-(aminomethyl)-3,5-dimethylbenzonitrile hydrochloride (57 mg, 0.29 mmol), Pd-PEPPSI-IPENT-Cl o-picoline (13 mg, 0.0145 mmol) and Cs₂CO₃ (183 mg, 0.56 mmol) in DMF (3 mL) was stirred at 100° C. for 10 h under N₂. After cooling to room temperature, the reaction was quenched with water (50 mL) and extracted with EtOAc (30 mL×3). The combined organic layers were washed with brine (30 mL), dried over Na₂SO₄, concentrated to give the crude, which was purified by Prep-TLC (eluent: DCM/MeOH=15/1) to give ethyl 3-(4-((4-cyano-2,6-dimethylbenzyl)amino)-6-((6-cyclopropylimidazo[1,2-a]pyridin-2-yl)methoxy)pyrimidin-2-yl)-2,2-dimethylpropanoate (30 mg, yield: 38%). ESI-MS [M+H]⁺: 553.1.

Synthesis of ethyl 3-(4-((4-carbamimidoyl-2,6-dimethylbenzyl)amino)-6-((6-cyclopropylimidazo[1,2-a]pyridin-2-yl)methoxy)pyrimidin-2-yl)-2,2-dimethylpropanoate. A solution of ethyl 3-(4-((4-cyano-2,6-dimethylbenzyl)amino)-6-((6-cyclopropylimidazo[1,2-a]pyridin-2-yl)methoxy)pyrimidin-2-yl)-2,2-dimethylpropanoate (30 mg, 0.054 mmol) in EtOH (5 mL) was bubbled dry HCl gas at room temperature for 6 h. The reaction mixture was concentrated, the residue was re-dissolved in EtOH (5 mL). NH₄HCO₃ (45 mg, 0.54 mmol) was added and the mixture was stirred at room temperature for 16 h. The reaction mixture was concentrated to give ethyl 3-(4-((4-carbamimidoyl-2,6-dimethylbenzyl)amino)-6-((6-cyclopropylimidazo[1,2-a]pyridin-2-yl)methoxy)pyrimidin-2-yl)-2,2-dimethylpropanoate (31 mg, yield: quant), which was used in the next step without purification. ESI-MS [M+H]+: 570.1.

Synthesis of 3-(4-((4-carbamimidoyl-2,6-dimethylbenzyl)amino)-6-((6-cyclopropylimidazo[1,2-a]pyridin-2-yl)methoxy)pyrimidin-2-yl)-2,2-dimethylpropanoic acid. A mixture of 3-(4-((4-carbamimidoyl-2,6-dimethylbenzyl)amino)-6-((6-cyclopropylimidazo[1,2-a]pyridin-2-yl)methoxy)pyrimidin-2-yl)-2,2-dimethylpropanoate (31 mg, 0.054 mmol) and NaOH (13 mg, 0.108 mmol) in EtOH/H₂O (2 mL/0.5 mL) was stirred at room temperature for 5 h. The reaction mixture was concentrated and the residue was purified by Prep-HPLC to give ethyl 3-(4-((4-carbamimidoyl-2,6-dimethylbenzyl)amino)-6-((6-cyclopropylimidazo[1,2-a]pyridin-2-yl)methoxy)pyrimidin-2-yl)-2,2-dimethylpropanoate as formate salt (7 mg, yield: 21%) and 3-(4-((4-carbamimidoyl-2,6-dimethylbenzyl)amino)-6-((6-cyclopropylimidazo[1,2-a]pyridin-2-yl)methoxy)pyrimidin-2-yl)-2,2-dimethylpropanoic acid as formate salt (5 mg, 14%).

ethyl 3-(4-((4-carbamimidoyl-2,6-dimethylbenzyl)amino)-6-((6-cyclopropylimidazo[1,2-a]pyridin-2-yl)methoxy)pyrimidin-2-yl)-2,2-dimethylpropanoate as formate salt: ESI-MS [M+H]⁺: 570.2. 1H NMR (400 MHz, DMSO-d₆) δ9.35 (s, 2H), 9.05 (s, 2H), 8.46 (s, 1H), 8.34 (s, 1H), 7.79 (s, 1H), 7.50 (s, 2H), 7.40 (d, J=8.0 Hz, 1H), 7.14 (s, 1H), 6.99 (d, J=8.0 Hz, 1H), 5.65 (s, 1H), 5.27 (s, 2H), 4.48 (s, 2H), 4.05-4.00 (m, 3H), 2.93 (s, 2H), 2.40 (s, 6H), 1.94-1.93 (m, 1H), 1.22 (s, 6 H), 1.10-1.08 (m, 3H), 0.93-0.91 (m, 2H), 0.68-0.67 (m, 2H).

3-(4-((4-carbamimidoyl-2,6-dimethylbenzyl)amino)-6-((6-cyclopropylimidazo[1,2-a]pyridin-2-yl)methoxy)pyrimidin-2-yl)-2,2-dimethylpropanoic acid as formate salt: ESI-MS [M+H]⁺: 542.2. 1H NMR (400 MHz, DMSO-d₆) δ11.67 (s, 1H), 9.05 (s, 2H), 8.72 (s, 2H), 8.45 (s, 1H), 8.33 (s, 1H), 7.82 (s, 1H), 7.41-7.38 (m, 3H), 7.04-7.69 (m, 2H), 5.61 (s, 1H), 5.30 (s, 2H), 4.51 (s, 2H), 2.89 (s, 2H), 2.36 (s, 6H), 1.96-1.90 (m, 1H), 1.15 (s, 6H), 0.95-0.90 (m, 2H), 0.70-0.66 (m, 2H).

### Example 94

### Synthesis of ethyl 2-(2-((6-(4-carbamimidoyl-2,6-dimethylbenzylamino)pyrimidin-4-yloxy)methyl)-6-cyclopropylimidazo[1,2-a]pyridin-8-ylacetate (I-94)

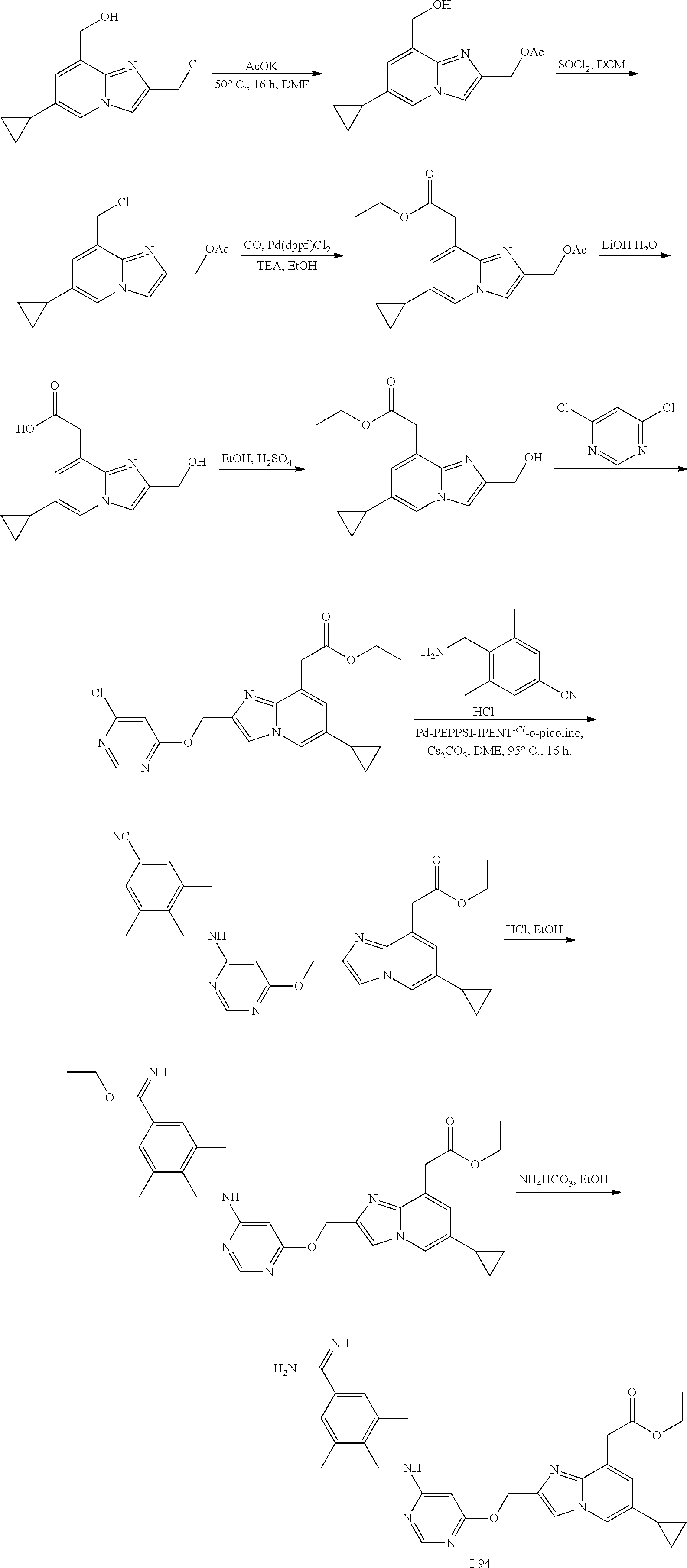

Synthesis of (6-cyclopropyl-8-(hydroxymethyl)imidazo[1,2-a]pyridin-2-yl)methyl acetate. A mixture of (2-(chloromethyl)-6-cyclopropylimidazo[1,2-a]pyridin-8-yl)methanol (4.3 g, 18.6 mmol) (see preparation in WO2019/178129) and AcOK (2.7 g, 28.0 mmol) in DMF (30 mL) was stirred at 50° C. for 3 h. After cooling to room temperature, the reaction was quenched with water (300 mL) and extracted with EtOAc (150 mL×3). The combined organic layers were washed with brine (150 mL), dried over Na₂SO₄, concentrated to give the crude, which was purified by column chromatography (eluent: DCM/MeOH=0-5%) to give (6-cyclopropyl-8-(hydroxymethyl)imidazo[1,2-a]pyridin-2-yl)methyl acetate (3.0 g, yield: 62%) as a brown oil. ESI-MS [M+H]+: 261.1

Synthesis of (8-(chloromethyl)-6-cyclopropylimidazo[1,2-a]pyridin-2-yl)methyl acetate. To a solution of (6-cyclopropyl-8-(hydroxymethyl)imidazo[1,2-a]pyridin-2-yl)methyl acetate (2.8 g, 10.8 mmol) in DCM (20 mL) was added SOCl₂ (1.0 mL) at 0° C. After the mixture was stirred at room temperature for 3 h, the mixture was concentrated to give (8-(chloromethyl)-6-cyclopropylimidazo[1,2-a]pyridin-2-yl)methyl acetate (3.0 g, yield: quant) as a yellow solid, which was used in the next step without purification. ESI-MS [M+H]+: 279.1.

Synthesis of ethyl 2-(2-(acetoxymethyl)-6-cyclopropylimidazo[1,2-a]pyridin-8-yl)acetate. A mixture of (8-(chloromethyl)-6-cyclopropylimidazo[1,2-a]pyridin-2-yl)methyl acetate (3.0 g, 10.8 mmol), Pd(dppf)Cl₂ (395 mg, 0.54 mmol) and TEA (5.0 mL) in EtOH (30.0 mL) was stirred at 80° C. under CO for 16 h. After cooling to room temperature, the reaction was quenched with water (30 mL) and extracted with EtOAc (30 mL×3). The combined organic layers were washed with brine (30 mL), dried over Na₂SO₄, concentrated to give the crude, which was purified by column chromatography (eluent: DCM/MeOH=0-5%) to give ethyl 2-(2-(acetoxymethyl)-6-cyclopropylimidazo[1,2-a]pyridin-8-yl)acetate (2.0 g, yield: 59%) as a light yellow oil. ESI-MS [M+H]⁺: 317.1.

Synthesis of 2-(6-cyclopropyl-2-(hydroxymethyl)imidazo[1,2-a]pyridin-8-yl)acetic acid. A solution of ethyl 2-(2-(acetoxymethyl)-6-cyclopropylimidazo[1,2-a]pyridin-8-yl)acetate (2.0 g, 6.3 mmol) and LiOH—H₂O (794 mg, 18.9 mmol) in MeOH/water (10 mL/10 mL) was stirred at room temperature for 16 h. The mixture was concentrated to give 2-(6-cyclopropyl-2-(hydroxymethyl)imidazo[1,2-a]pyridin-8-yl)acetic acid (2.0 g, crude) as a yellow oil. which was used in the next step without further purification. ESI-MS [M+H]+: 247.1.

Synthesis of ethyl 2-(6-cyclopropyl-2-(hydroxymethyl)imidazo[1,2-a]pyridin-8-yl)acetate. To a solution of 2-(6-cyclopropyl-2-(hydroxymethyl)imidazo[1,2-a]pyridin-8-yl)acetic acid (2.0 g, crude) in EtOH (30.0 mL) was added conc. H₂SO₄ (0.5 mL) and the mixture was stirred at reflux for 5 h. After cooling to room temperature, the reaction was quenched with NaHCO₃ (sat. aq., 100 mL) and extracted with EtOAc (100 mL×3). The combined organic layers were washed with brine (50 mL), dried over Na₂SO₄, concentrated to give the crude, which was purified by column chromatography (eluent: DCM/MeOH=0-8%) to give ethyl 2-(6-cyclopropyl-2-(hydroxymethyl)imidazo[1,2-a]pyridin-8-yl)acetate (1.5 g, 87% over 2 steps) as a yellow solid. ESI-MS [M+H]+: 275.1.

Synthesis of ethyl 2-(2-((6-chloropyrimidin-4-yloxy)methyl)-6-cyclopropylimidazo[1,2-a]pyridin-8-yl)acetate 1-((6-cyclopropylimidazo[1,2-a]pyridin-2-yl)methyl)-5-(morpholinomethyl)-1H-pyrazole-4-carboxylate. To a solution of ethyl 2-(6-cyclopropyl-2-(hydroxymethyl)imidazo[1,2-a]pyridin-8-yl)acetate (550 mg, 2.0 mmol) in dry THF (20 mL) was added NaH (160 mg, 4.0 mmol, 60% in mineral oil) slowly at 0° C. After stirring at for 0.5 h room temperature, 4,6-dichloropyrimidine (447 mg, 3.0 mmol) in dry THF (5 mL) was added and the mixture was stirred at 60° C. for 2 h. The reaction was quenched with NH₄Cl (sat. aq., 100 mL) and extracted with DCM/MeOH (10/1, 50 mL×3). The combined organic layers were washed with brine (50 mL), dried over Na₂SO₄, concentrated to give the crude, which was purified by Prep-TLC (eluent: DCM/MeOH=15/1) to give ethyl 2-(2-((6-chloropyrimidin-4-yloxy)methyl)-6-cyclopropylimidazo[1,2-a]pyridin-8-yl)acetate (150 mg, yield: 19%) as a yellow solid. ESI-MS [M+H]+: 387.1.

Synthesis of ethyl 2-(2-((6-(4-cyano-2,6-dimethylbenzylamino)pyrimidin-4-yloxy)methyl)-6-cyclopropylimidazo[1,2-a]pyridin-8-yl)acetate. A mixture of ethyl 2-(2-((6-chloropyrimidin-4-yloxy)methyl)-6-cyclopropylimidazo[1,2-a]pyridin-8-yl)acetate (130 mg, 0.34 mmol), 4-(aminomethyl)-3,5-dimethylbenzonitrile hydrochloride (86 mg, 0.44 mmol), Pd-PEPPSI-IPENT-Cl-o-picoline (29 mg, 0.034 mmol) and Cs₂CO₃ (330 mg, 1.02 mmol) in DMF (10 mL) was stirred at 95° C. for 16 h under N₂. After cooling to room temperature, the reaction was quenched with water (100 mL) and extracted with EtOAc (50 mL×3). The combined organic layers were washed with brine (50 mL), dried over Na₂SO₄, concentrated to give the crude, which was purified by Prep-TLC (eluent: DCM/MeOH=0-4%) to give ethyl 2-(2-((6-(4-cyano-2,6-dimethylbenzylamino)pyrimidin-4-yloxy)methyl)-6-cyclopropylimidazo[1,2-a]pyridin-8-yl)acetate (110 mg, yield: 64%) as a yellow solid. ESI-MS [M+H]+: 511.1.

Synthesis of ethyl 2-(6-cyclopropyl-2-((6-(4-(ethoxy(imino)methyl)-2,6-dimethylbenzylamino)pyrimidin-4-yloxy)methyl)imidazo[1,2-a]pyridin-8-yl)acetate. A mixture of ethyl 2-(2-((6-(4-cyano-2,6-dimethylbenzylamino)pyrimidin-4-yloxy)methyl)-6-cyclopropylimidazo[1,2-a]pyridin-8-yl)acetate (102 mg, 0.2 mmol) in EtOH (10 mL) was bubbled dry HCl gas at room temperature for 2 h. The mixture was concentrated to give ethyl 2-(6-cyclopropyl-2-((6-(4-(ethoxy(imino)methyl)-2,6-dimethylbenzylamino)pyrimidin-4-yloxy)methyl)imidazo[1,2-a]pyridin-8-yl)acetate (110 mg, yield: quant) as a yellow solid, which was used in the next step without purification. ESI-MS [M+H]+: 557.1.

Synthesis of ethyl 2-(2-((6-(4-carbamimidoyl-2,6-dimethylbenzylamino)pyrimidin-4-yloxy)methyl)-6-cyclopropylimidazo[1,2-a]pyridin-8-yl)acetate. A mixture of ethyl 2-(6-cyclopropyl-2-((6-(4-(ethoxy(imino)methyl)-2,6-dimethylbenzylamino)pyrimidin-4-yloxy)methyl)imidazo[1,2-a]pyridin-8-yl)acetate (110 mg, 0.2 mmol) and NH₄HCO₃ (142 mg, 1.80 mmol) in EtOH (10 mL) was stirred at room temperature for 16 h. The mixture was concentrated and purified by Prep-HPLC to give ethyl 2-(2-((6-(4-carbamimidoyl-2,6-dimethylbenzylamino)pyrimidin-4-yloxy)methyl)-6-cyclopropylimidazo[1,2-a]pyridin-8-yl)acetate as formate salt (50 mg, yield: 43%) as a white solid. ESI-MS [M+H]+: 528.1. ¹H NMR (400 MHz, DMSO) δ9.42-8.91 (m, 2H), 8.47 (s, 1H), 8.26 (s, 2H), 7.81 (s, 1H), 7.50 (s, 2H), 7.29 (t, J=4.7 Hz, 1H), 6.94 (s, 1H), 5.85 (s, 1H), 5.33 (s, 2H), 4.51 (s, 2H), 4.09-4.06 (m, 2H), 3.90 (s, 2H), 2.41 (s, 6H), 1.95-1.89 (m, 1H), 1.18 (t, J=7.1 Hz, 3H), 0.96-0.85 (m, 2H), 0.72-0.58 (m, 2H).

### Example 95

### Synthesis of 2-(2-((6-(4-carbamimidoyl-2,6-dimethylbenzylamino)pyrimidin-4-yloxy)methyl)-6-cyclopropylimidazo[1,2-a]pyridin-8-yl)acetic acid (I-95)

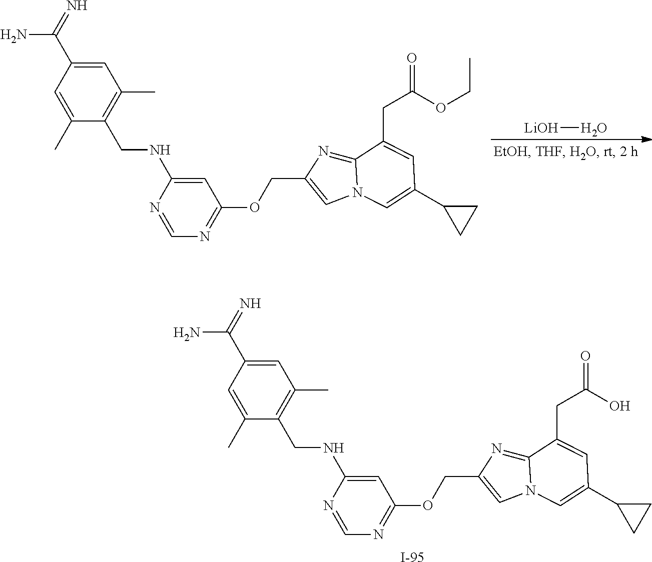

A mixture of ethyl 2-(2-((6-(4-carbamimidoyl-2,6-dimethylbenzylamino)pyrimidin-4-yloxy)methyl)-6-cyclopropylimidazo[1,2-a]pyridin-8-yl)acetate (26 mg, 0.05 mmol) (synthesis reported in Example 94) and LiOH—H₂O (6.3 mg, 0.15 mmol) in EtOH/THF/water (1 mL/1 mL/1 mL) was stirred at room temperature for 2 h. The mixture was concentrated and purified by Prep-HPLC to give 2-(2-((6-(4-carbamimidoyl-2,6-dimethylbenzylamino)pyrimidin-4-yloxy)methyl)-6-cyclopropylimidazo[1,2-a]pyridin-8-yl)acetic acid as formate salt (10 mg, yield: 36%) as a white solid. ESI-MS [M+H]⁺: 500.2. ¹H NMR (400 MHz, DMSO) δ11.58 (s, 1H), 8.78 (s, 2H), 8.37 (s, 1H), 8.27 (s, 1H), 8.19 (s, 1H), 7.81 (s, 1H), 7.47 (s, 2H), 7.21 (s, 1H), 6.84 (s, 1H), 5.85 (s, 1H), 5.30 (s, 2H), 4.49 (s, 2H), 3.66 (s, 2H), 2.36 (s, 6H), 1.91-1.87 (m, 1H), 0.99-0.85 (m, 2H), 0.73-0.60 (m, 2H).

### Example 96

### Synthesis of ethyl 3-(2-(((6-((4-carbamimidoyl-2,6-dimethylbenzyl)amino)pyrimidin-4-yl)oxy)methyl)-6-cyclopropylimidazo[1,2-a]pyridin-8-yl)propanoate (I-96)

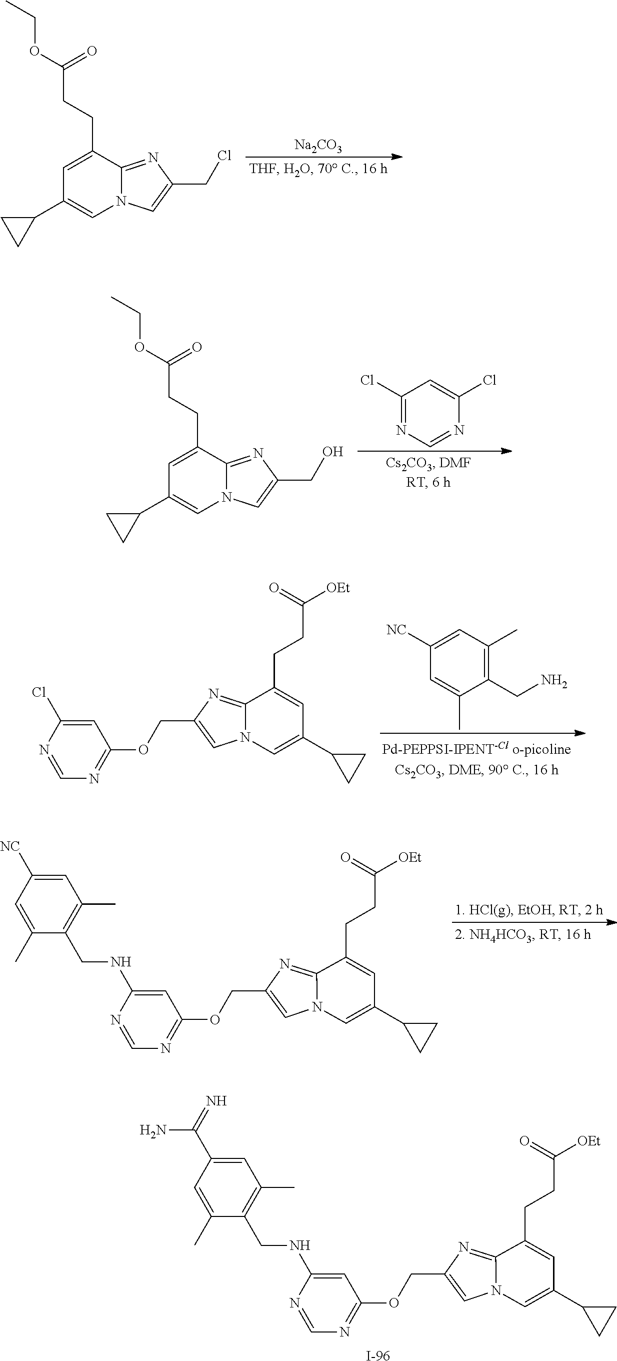

Synthesis of ethyl 3-(6-cyclopropyl-2-(hydroxymethyl)imidazo[1,2-a]pyridin-8-yl)propanoate. A mixture of ethyl 3-(2-(chloromethyl)-6-cyclopropylimidazo[1,2-a]pyridin-8-yl)propanoate (307 mg, 1 mmol) (see preparation in WO2019/178129) and Na₂CO₃ (210 mg, 2 mmol) in THF (20 mL) and H₂O (20 mL) was stirred at 70° C. for 16 h. After cooling to room temperature, the reaction mixture was quenched with water (30 mL) and extracted with EtOAc (30 mL×5). The combined organic layers were washed with brine (30 mL), dried over Na₂SO₄, and concentrated in vacuo to give ethyl 3-(6-cyclopropyl-2-(hydroxymethyl)imidazo[1,2-a]pyridin-8-yl)propanoate as a white solid (290 mg, yield: quant), which was used into next step without further purification. ESI-MS [M+H]+: 289.2.

Synthesis of ethyl 3-(2-(((6-chloropyrimidin-4-yl)oxy)methyl)-6-cyclopropylimidazo[1,2-a]pyridin-8-yl)propanoate. To a mixture of ethyl 3-(6-cyclopropyl-2-(hydroxymethyl)imidazo[1,2-a]pyridin-8-yl)propanoate (290 mg, 1 mmol) and 4,6-dichloropyrimidine (224 mg, 1.5 mmol) in DMF (10 mL) was added Cs₂CO₃ (981 mg, 3 mmol). The mixture was stirred at room temperature for 6 h. The reaction was quenched with H₂O (50 mL) and extracted with EtOAc (50 mL×3). The combined organic layers were washed with brine (50 mL), dried over Na₂SO₄, concentrated in vacuo to give the crude, which was purified column chromatography (eluent: EtOAc/PE=0-50%) to give ethyl 3-(2-(((6-chloropyrimidin-4-yl)oxy)methyl)-6-cyclopropylimidazo[1,2-a]pyridin-8-yl)propanoate (200 mg, yield: 50%) as a yellow oil. ESI-MS [M+H]⁺: 401.1.

Synthesis of ethyl 3-(2-(((6-((4-cyano-2,6-dimethylbenzyl)amino)pyrimidin-4-yl)oxy)methyl)-6-cyclopropylimidazo[1,2-a]pyridin-8-yl)propanoate. To a solution of ethyl 3-(2-(((6-chloropyrimidin-4-yl)oxy)methyl)-6-cyclopropylimidazo[1,2-a]pyridin-8-yl)propanoate (200 mg, 0.5 mmol), 4-(aminomethyl)-3,5-dimethylbenzonitrile hydrochloride (195 mg, 1.0 mmol) in DME (5 mL) was added Pd-PEPPSI-IPENT-^{Cl} o-picoline (42 mg, 0.05 mmol) and Cs₂CO₃ (489 mg, 1.5 mmol). The resulting mixture was stirred at 90° C. for 16 h. After cooling to room temperature, the reaction was quenched with water (50 mL) and extracted with EtOAc (50 mL×3). The combined organic layers were washed with brine (50 mL), dried over Na₂SO₄, concentrated to give the crude, which was purified by Prep-TLC (eluent: DCM/MeOH=20/1) to give ethyl 3-(2-(((6-((4-cyano-2,6-dimethylbenzyl)amino)pyrimidin-4-yl)oxy)methyl)-6-cyclopropylimidazo[1,2-a]pyridin-8-yl)propanoate (180 mg, yield: 69%) as a yellow oil. ESI-MS [M+H]+: 525.2.

Synthesis of ethyl 3-(2-(((6-((4-carbamimidoyl-2,6-dimethylbenzyl)amino)pyrimidin-4-yl)oxy)methyl)-6-cyclopropylimidazo[1,2-a]pyridin-8-yl)propanoate. To a mixture of ethyl 3-(2-(((6-((4-cyano-2,6-dimethylbenzyl)amino)pyrimidin-4-yl)oxy)methyl)-6-cyclopropylimidazo[1,2-a]pyridin-8-yl)propanoate (180 mg, 0.34 mmol) in EtOH (3 mL) was bubbled HCl(g) for 2 h at room temperature. The mixture was concentrated, the residue was re-dissolved in EtOH (3 mL) and NH₄HCO₃ (271 mg, 3.4 mmol) was added. After the mixture was stirred at room temperature for 16 h, the reaction mixture was concentrated, the residue was purified by Prep-HPLC to give ethyl 3-(2-(((6-((4-carbamimidoyl-2,6-dimethylbenzyl)amino)pyrimidin-4-yl)oxy)methyl)-6-cyclopropylimidazo[1,2-a]pyridin-8-yl)propanoate (110 mg, yield: 60%) as a white solid. ESI-MS [M+H]⁺: 542.2. ¹H NMR (400 MHz, DMSO) δ9.24 (s, 2H), 9.06 (s, 2H), 8.52 (s, 1H), 8.30 (s, 1H), 8.17 (s, 1H), 7.52 (s, 2H), 7.45-7.40 (m, 2H), 5.92 (s, 1H), 5.51 (s, 2H), 4.54 (s, 2H), 4.06 (q, J=7.1 Hz, 2H), 3.15 (t, J=7.6 Hz, 2H), 2.81 (t, J=7.7 Hz, 2H), 2.42 (s, 6H), 2.05-1.98 (m, 1H), 1.15 (t, J=7.1 Hz, 3H), 1.06-0.98 (m, 2H), 0.81-0.73 (m, 2H).

### Example 97

### Synthesis of 3-(2-(((6-((4-carbamimidoyl-2,6-dimethylbenzyl)amino)pyrimidin-4-yl)oxy)methyl)-6-cyclopropylimidazo[1,2-a]pyridin-8-yl)propanoic acid (I-97)

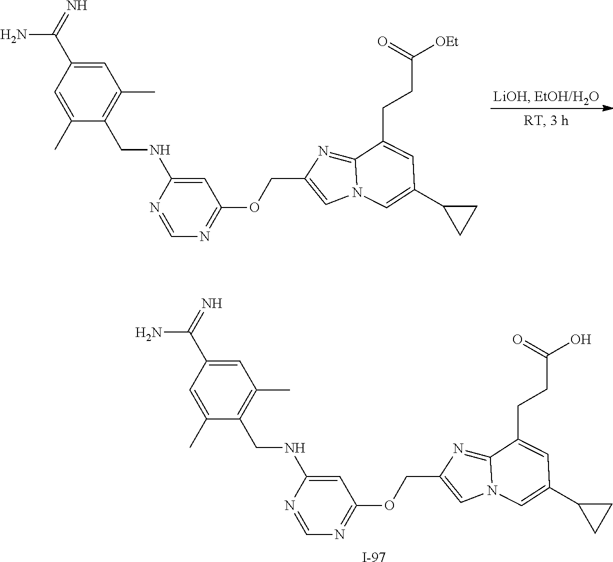

To a mixture of ethyl 3-(2-(((6-((4-carbamimidoyl-2,6-dimethylbenzyl)amino)pyrimidin-4-yl)oxy)methyl)-6-cyclopropylimidazo[1,2-a]pyridin-8-yl)propanoate (54 mg, 0.1 mmol) in EtOH/H₂O (2 mL/0.2 mL) was added LiOH—H₂O (13 mg, 0.3 mmol). The mixture was stirred at room temperature for 3 h then concentrated. The residue was purified by Prep-HPLC to give 3-(2-(((6-((4-carbamimidoyl-2,6-dimethylbenzyl)amino)pyrimidin-4-yl)oxy)methyl)-6-cyclopropylimidazo[1,2-a]pyridin-8-yl)propanoic acid as formate salt (28 mg, yield: 50%) as a white solid. ESI-MS [M+H]⁺: 514.2. ¹H NMR (400 MHz, DMSO) δ12.31 (s, 1H), 9.61 (s, 2H), 9.13 (s, 2H), 8.26 (s, 1H), 8.19 (s, 1H), 7.80 (s, 1H), 7.50 (s, 2H), 7.30 (t, J=4.7 Hz, 1H), 6.83 (s, 1H), 5.87 (s, 1H), 5.35 (s, 2H), 4.52 (s, 2H), 3.05 (t, J=7.7 Hz, 2H), 2.69 (t, J=7.8 Hz, 2H), 2.43 (s, 6H), 1.92-1.85 (m, 1H), 0.93-0.86 (m, 2H), 0.69-0.63 (m, 2H).

### Example 98

### Synthesis of 4-(((5-(((6-cyclopropylimidazo[1,2-a]pyridin-2-yl)methyl)amino)pyridazin-3-yl)amino)methyl)-3,5-dimethylbenzimidamide (I-98)

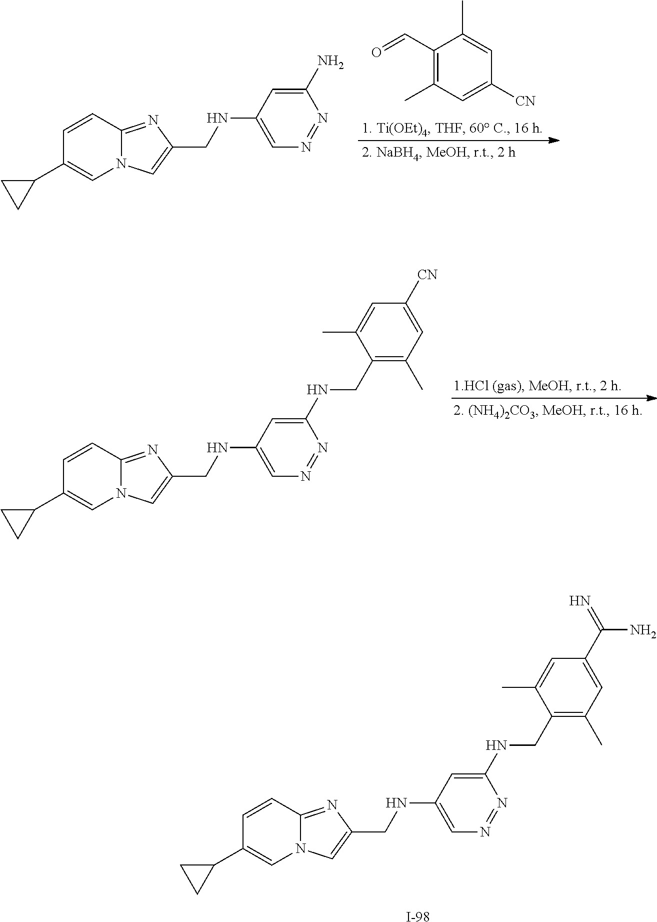

Synthesis of (4-(((5-(((6-cyclopropylimidazo[1,2-a]pyridin-2-yl)methyl)amino)pyridazin-3-yl)amino)methyl)-3,5-dimethylbenzonitrile. To a solution of N⁵-((6-cyclopropylimidazo[1,2-a]pyridin-2-yl)methyl)pyridazine-3,5-diamine (100 mg, 0.36 mmol) and 4-formyl-3,5-dimethylbenzonitrile (57 mg, 0.36 mmol) in THF (10 mL) was added Ti(OEt)₄ (410 mg, 1.8 mmol). The mixture was stirred at 60° C. for 16 h. After cooling to room temperature, a solution of NaBH₄ (27 mg, 0.72 mmol) in MeOH (10 mL) was added and the mixture was stirred at room temperature for 2 h. The reaction was quenched with water (50 mL) and extracted with EtOAc (30 mL×3). The combined organic layers were washed with brine (50 mL), dried over anhydrous Na₂SO₄, and concentrated in vacuo to give the crude, which was purified by (eluent: DCM/MeOH=20/1) to give 4-(((5-(((6-cyclopropylimidazo[1,2-a]pyridin-2-yl)methyl)amino)pyridazin-3-yl)amino)methyl)-3,5-dimethylbenzonitrile (45 mg, yield: 30%) as a white solid. ESI-MS [M+H]+: 424.2.

Synthesis of 4-(((5-(((6-cyclopropylimidazo[1,2-a]pyridin-2-yl)methyl)amino)pyridazin-3-yl)amino)methyl)-3,5-dimethylbenzimidamide. To a solution of 4-(((5-(((6-cyclopropylimidazo[1,2-a]pyridin-2-yl)methyl)amino)pyridazin-3-yl)amino)methyl)-3,5-dimethylbenzonitrile (45 mg, 0.11 mmol) in MeOH (5 mL) was bubbled dry HCl gas with stirring at room temperature for 2 h. The reaction was concentrated to give the crude, which was re-dissolved in MeOH (5 mL). (NH₄)₂CO₃ (53 mg, 0.55 mmol) was added and the resulting mixture was stirred at room temperature for 16 h. The reaction was concentrated to give the crude, which was purified by Prep-HPLC to give 4-(((5-(((6-cyclopropylimidazo[1,2-a]pyridin-2-yl)methyl)amino)pyridazin-3-yl)amino)methyl)-3,5-dimethylbenzimidamide as formate salt (24 mg, yield: 45%) as a white solid. ESI-MS [M+H]+: 441.2. ¹H NMR (400 MHz, DMSO) δ10.93 (s, 2H), 8.93 (s, 2H), 8.33-8.30 (m, 2H), 8.07 (d, J=2.4 Hz, 1H), 7.64 (s, 1H), 7.48 (s, 2H), 7.37 (d, J=9.3 Hz, 1H), 7.07 (t, J=5.6 Hz, 1H), 6.97-6.95 (m, 1H), 6.40 (s, 1H), 5.78 (d, J=2.3 Hz, 1H), 4.50 (d, J=4.8 Hz, 2H), 4.29 (d, J=5.6 Hz, 2H), 2.41 (s, 6H), 1.91-1.87 (m, 1H), 0.94-0.84 (m, 2H), 0.71-0.61 (m, 2H).

### Example 99

### Synthesis of 4-(((6-(((6-cyclopropylimidazo[1,2-a]pyridin-2-yl)methyl)amino)pyrimidin-4-yl)amino)methyl)-3,5-dimethylbenzimidamide (I-99)

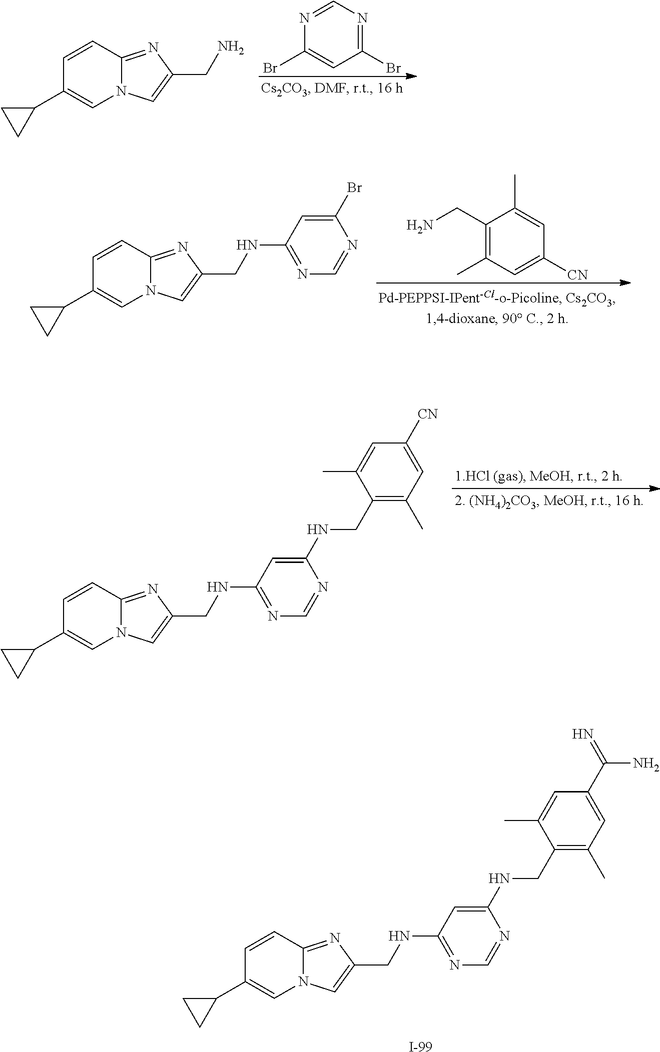

Synthesis of 6-bromo-N-((6-cyclopropylimidazo[1,2-a]pyridin-2-yl)methyl)pyrimidin-4-amine. A mixture of (6-cyclopropylimidazo[1,2-a]pyridin-2-yl)methanamine (200 mg, 1.1 mmol), 4,6-dibromopyrimidine (262 mg, 1.1 mmol) and Cs₂CO₃ (1.1 g, 3.3 mmol) in DMF (10 mL) was stirred at room temperature for 16 h. The reaction was quenched with water (50 mL) and extracted with EtOAc (30 mL×3). The combined organic layers were washed with brine (50 mL), dried over anhydrous Na₂SO₄, and concentrated to give the crude product, which was purified by Prep-TLC (eluent: DCM/MeOH=30/1) to give 6-bromo-N-((6-cyclopropylimidazo[1,2-a]pyridin-2-yl)methyl)pyrimidin-4-amine (250 mg, yield: 66%) as a yellow solid. ESI-MS [M+H]+: 344.2.

Synthesis of 4-(((6-(((6-cyclopropylimidazo[1,2-a]pyridin-2-yl)methyl)amino)pyrimidin-4-yl)amino)methyl)-3,5-dimethylbenzonitrile. To a solution of 6-bromo-N-((6-cyclopropylimidazo[1,2-a]pyridin-2-yl)methyl)pyrimidin-4-amine (200 mg, 0.58 mmol), 4-(aminomethyl)-3,5-dimethylbenzonitrile (93 mg, 0.58 mmol) and Cs₂CO₃ (567 mg, 1.74 mmol) in 1,4-dioxane (5 mL) was added Pd-PEPPSI-IPent-^{Cl}-o-Picoline (50 mg, 0.06 mmol). After the mixture was stirred at 90° C. for 2 h under N₂, the reaction was cooled to room temperature. The mixture was filtered through Celite® and the filter cake was washed with DCM/MeOH (10/1, 30 mL). The filtrate was concentrated to give the crude, which was purified by Prep-TLC (eluent: DCM/MeOH=20/1) to give 4-(((6-(((6-cyclopropylimidazo[1,2-a]pyridin-2-yl)methyl)amino)pyrimidin-4-yl)amino)methyl)-3,5-dimethylbenzonitrile (60 mg, yield: 24%) as a white solid. ESI-MS [M+H]+: 424.2.

Synthesis of 4-(((6-(((6-cyclopropylimidazo[1,2-a]pyridin-2-yl)methyl)amino)pyrimidin-4-yl)amino)methyl)-3,5-dimethylbenzimidamide. To a solution of 4-(((6-(((6-cyclopropylimidazo[1,2-a]pyridin-2-yl)methyl)amino)pyrimidin-4-yl)amino)methyl)-3,5-dimethylbenzonitrile (60 mg, 0.14 mmol) in MeOH (5 mL) was bubbled dry HCl gas with stirring at room temperature for 2 h. The reaction was concentrated to give the crude, which was re-dissolved in MeOH (5 mL). (NH₄)₂CO₃ (134 mg, 1.4 mmol) was added and the mixture was stirred at room temperature for 16 h. The reaction was concentrated to give the crude, which was purified by Prep-HPLC to give 4-(((6-(((6-cyclopropylimidazo[1,2-a]pyridin-2-yl)methyl)amino)pyrimidin-4-yl)amino)methyl)-3,5-dimethylbenzimidamide as formate salt (7.5 mg, yield: 10%) as a white solid. ESI-MS [M+H]+: 441.2. ¹H NMR (400 MHz, DMSO) δ8.44 (s, 5H), 8.29 (s, 1H), 7.99 (s, 1H), 7.58 (s, 1H), 7.48 (s, 2H), 7.35 (d, J=9.3 Hz, 1H), 7.05 (s, 1H), 6.94 (dd, J=9.3, 1.7 Hz, 1H), 6.74 (s, 1H), 5.50 (s, 1H), 4.40-4.43 (m, 4H), 2.40 (s, 6H), 1.98-1.83 (m, 1H), 0.97-0.84 (m, 2H), 0.74-0.59 (m, 2H).

### Example 100

### Synthesis of 2-(7-chloroimidazo[1,5-a]pyridin-1-yl)-N-(6-(((6-cyclopropylimidazo[1,2-a]pyridin-2-ylmethyl)amino)-2-(2-methoxyethoxy pyrimidin-4-yl)acetamide (I-100)

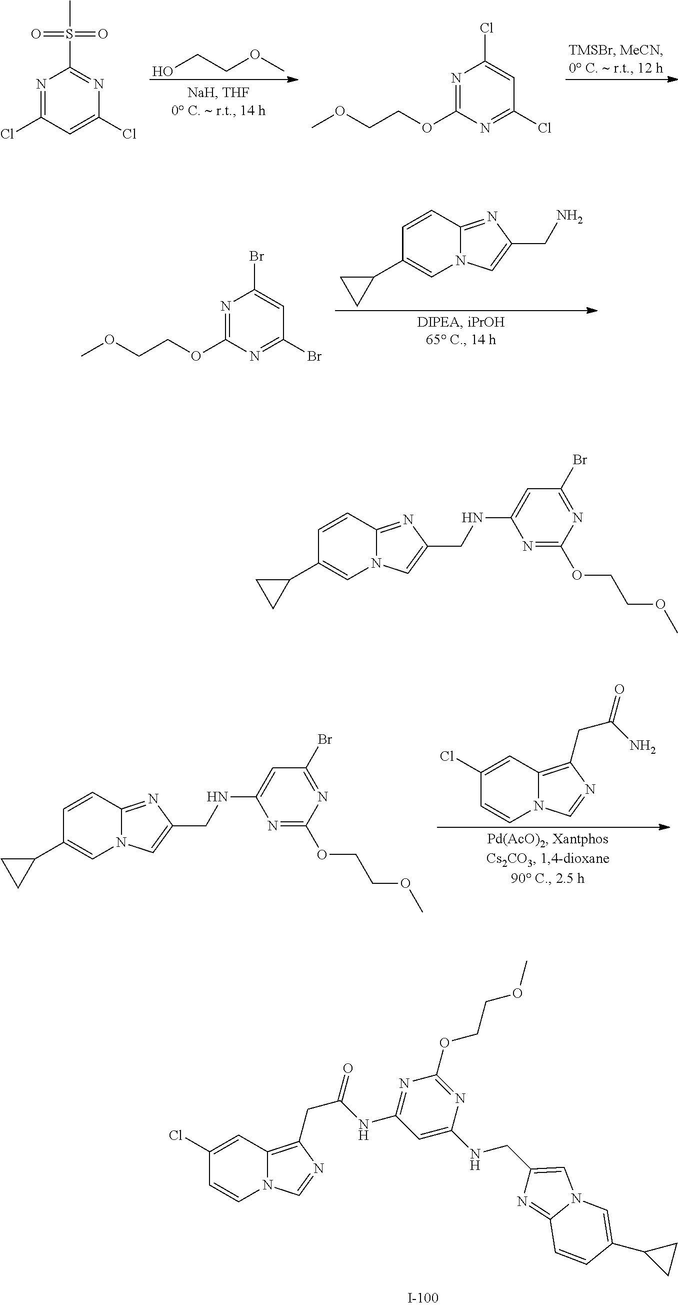

Synthesis of 4,6-dichloro-2-(2-methoxyethoxy)pyrimidine. To a solution of 2-methoxyethan-1-ol (5 g, 66 mmol) in THF (50 mL) was added NaH (3.2 g, 80 mmol, 60% dispersion in mineral oil) slowly at 0° C. The reaction mixture was stirred at 0° C. for 30 min. Then a solution of 4,6-dichloro-2-(methylsulfonyl)pyrimidine (10 g, 44 mmol) in THF (20 mL) was added thereto. The resulting mixture was stirred at room temperature for 14 h. The reaction was quenched with NH₄Cl (sat. aq., 100 mL), extracted with EtOAc (100 mL×3). The combined organic layers were washed with brine (150 mL), dried over anhydrous Na₂SO₄, concentrated in vacuo to give the crude, which was purified with silica gel column chromatography eluting with a gradient of 0-10% EtOAc in PE to give 4,6-dichloro-2-(2-methoxyethoxy)pyrimidine (2.5 g, yield: 26%) as a yellow solid. ESI-MS [M+H]+: 223.2.

Synthesis of 4,6-dibromo-2-(2-methoxyethoxy)pyrimidine. To a solution of 4,6-dichloro-2-(2-methoxyethoxy)pyrimidine (400 mg, 1.8 mmol) in MeCN (10 mL) was added TMSBr (1.4 g, 9 mmol). The resulting reaction was stirred at room temperature for 12 h under N₂. H₂O (30 mL) was added and extracted with EtOAc (50 mL×3). The combined organic layers were washed with brine (50 mL), dried over anhydrous Na₂SO₄, concentrated in vacuo to give the crude, which was purified with Prep-TLC (eluent: PE/EtOAc=(eluent: PE/EtOAc=10/1) to give 4,6-dibromo-2-(2-methoxyethoxy)pyrimidine (410 mg, yield: 73%) as a yellow solid. ESI-MS [M+H]+: 311.3.

Synthesis of 6-bromo-N-((6-cyclopropylimidazo[1,2-a]pyridin-2-yl)methyl)-2-(2-methoxyethoxy)pyrimidin-4-amine. A solution of 4,6-dibromo-2-(2-methoxyethoxy)pyrimidine (200 mg, 0.64 mmol), (6-cyclopropylimidazo[1,2-a]pyridin-2-yl)methanamine (180 mg, 0.96 mmol) and DIPEA (330 mg, 2.56 mmol) in i-PrOH (10 mL) was stirred at 65° C. for 14 h. The reaction was cooled to room temperature, concentrated in vacuo to give the crude, which was purified with Prep-TLC (eluent: DCM/MeOH=15/1) to give 6-bromo-N-((6-cyclopropylimidazo[1,2-a]pyridin-2-yl)methyl)-2-(2-methoxyethoxy)pyrimidin-4-amine (170 mg, 64%) as a yellow solid. ESI-MS [M+H]⁺: 418.2.

Synthesis of 2-(7-chloroimidazo[1,5-a]pyridin-1-yl)-N-(6-(((6-cyclopropylimidazo[1,2-a]pyridin-2-yl)methyl)amino)-2-(2-methoxyethoxy)pyrimidin-4-yl)acetamide. A mixture of 6-bromo-N-((6-cyclopropylimidazo[1,2-a]pyridin-2-yl)methyl)-2-(2-methoxyethoxy)pyrimidin-4-amine (170 mg, 0.41 mmol), 2-(7-chloroimidazo[1,5-a]pyridin-1-yl)acetamide (130 mg, 0.62 mmol), Pd(OAc)₂ (9 mg, 0.041 mmol), Xantphos (47 mg, 0.082 mmol) and Cs₂CO₃ (402 mg, 1.23 mmol) in 1,4-dioxane (10 mL) was stirred at 90° C. for 2.5 h under N₂. After cooled to room temperature, the reaction was quenched with H₂O (25 mL), extracted with EtOAc (30 mL×3). The combined organic layers were washed with brine (30 mL), dried over anhydrous Na₂SO₄, concentrated in vacuo to give the crude, which was purified with Prep-HPLC to give 2-(7-chloroimidazo[1,5-a]pyridin-1-yl)-N-(6-(((6-cyclopropylimidazo[1,2-a]pyridin-2-yl)methyl)amino)-2-(2-methoxyethoxy)pyrimidin-4-yl)acetamide (14 mg, 6%) as a white solid. ESI-MS [M+H]+: 547.2. ¹H NMR (400 MHz, MeOD) δ8.40 (s, 1H), 8.30 (s, 1H), 8.19 (d, J=7.1 Hz, 1H), 7.86 (s, 1H), 7.64-7.60 (m, 2H), 7.51 (d, J=9.1 Hz, 1H), 7.02 (s, 1H), 6.64 (d, J=7.5 Hz, 1H), 4.73 (s, 2H), 4.37 (t, J=4.0 Hz, 2H), 3.98 (s, 2H), 3.64 (t, J=4.0 Hz, 2H), 3.33 (s, 2H), 2.07-1.99 (m, 1H), 1.09-1.03 (m, 2H), 0.81-0.76 (m, 2H).

### Example 101

### Synthesis of 2-(7-chloroimidazo[1,5-a]pyridin-1-yl)-N-(6-(((6-cyclopropylimidazo[1,2-a]pyridin-2-yl)methyl)amino)-2-(trifluoromethyl)pyrimidin-4-yl)acetamide (I-101)

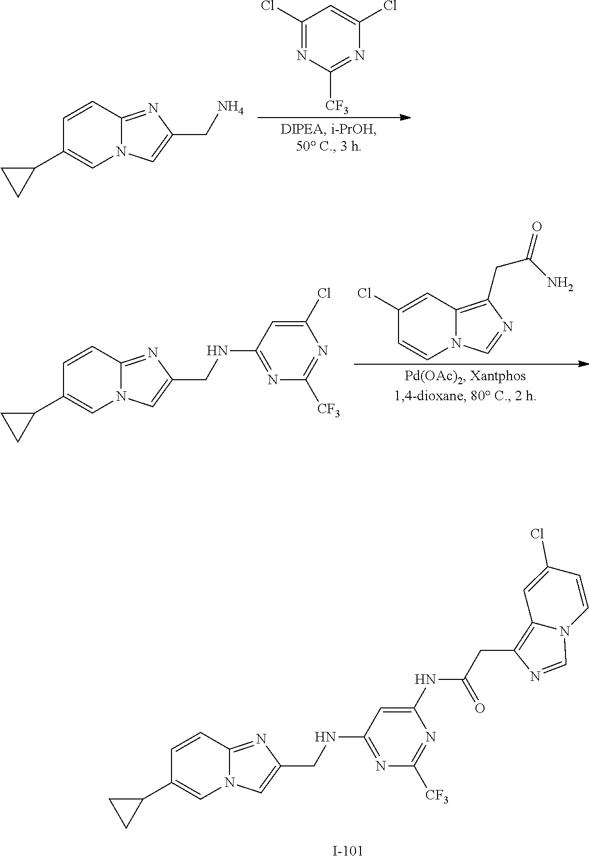 [SMILES: NCc1cn2cc(C3CC3)ccc2n1 ; FC(F)(F)c1nc(Cl)cc(Cl)n1 ; FC(F)(F)c1nc(Cl)cc(NCc2cn3cc(C4CC4)ccc3n2)n1 ; NC(=O)Cc1ncn2ccc(Cl)cc12 ; O=C(Cc1ncn2ccc(Cl)cc12)Nc1cc(NCc2cn3cc(C4CC4)ccc3n2)nc(C(F)(F)F)n1]

Synthesis of 6-chloro-N-((6-cyclopropylimidazo[1,2-a]pyridin-2-yl)methyl)-2-(trifluoromethyl)pyrimidin-4-amine. A mixture of (6-cyclopropylimidazo[1,2-a]pyridin-2-yl)methanamine (150 mg, 0.8 mmol), 4,6-dichloro-2-(trifluoromethyl)pyrimidine (173 mg, 0.8 mmol) and DIPEA (310 mg, 2.4 mmol) in i-PrOH (10 mL) was stirred at 50° C. for 3 h. After the reaction mixture was cooled to room temperature, the reaction was quenched with water (50 mL) and extracted with EtOAc (30 mL×3). The combined organic layers were washed with brine (50 mL), dried over anhydrous Na₂SO₄, and concentrated to give the crude product, which was purified by Prep-TLC (eluent: DCM/MeOH=30/1) to give 6-chloro-N-((6-cyclopropylimidazo[1,2-a]pyridin-2-yl)methyl)-2-(trifluoromethyl)pyrimidin-4-amine (120 mg, yield: 41%) as a yellow solid. ESI-MS [M+H]⁺: 368.2.

Synthesis of 2-(7-chloroimidazo[1,5-a]pyridin-1-yl)-N-(6-(((6-cyclopropylimidazo[1,2-a]pyridin-2-yl)methyl)amino)-2-(trifluoromethyl)pyrimidin-4-yl)acetamide. To a mixture of 6-chloro-N-((6-cyclopropylimidazo[1,2-a]pyridin-2-yl)methyl)-2-(trifluoromethyl)pyrimidin-4-amine (60 mg, 0.16 mmol), 2-(7-chloroimidazo[1,5-a]pyridin-1-yl)acetamide (33 mg, 0.16 mmol) and Cs₂CO₃ (156 mg, 0.48 mmol) in 1,4-dioxane (5 mL) was added Xantphos (18 mg, 0.032 mmol) and Pd(OAc)₂ (4 mg, 0.016 mmol). The mixture was stirred at 80° C. for 2 h under N₂. The reaction mixture was cooled to room temperature. The mixture was filtered through Celite® and the filter cake was washed with DCM/MeOH (10/1, 30 mL). The filtrate was concentrated to give the crude, which was purified by Prep-HPLC to give 2-(7-chloroimidazo[1,5-a]pyridin-1-yl)-N-(6-(((6-cyclopropylimidazo[1,2-a]pyridin-2-yl)methyl)amino)-2-(trifluoromethyl)pyrimidin-4-yl)acetamide (4 mg, yield: 5%) as a white solid. ESI-MS [M+H]⁺: 541.2. ¹H NMR (400 MHz, DMSO) δ10.86 (s, 1H), 8.46-8.45 (m, 1H), 8.42-8.18 (m, 3H), 7.82 (s, 1H), 7.64 (s, 1H), 7.48-7.30 (m, 2H), 6.96 (d, J=9.3 Hz, 1H), 6.66 (d, J=7.2 Hz, 1H), 4.62 (s, 2H), 3.97 (s, 2H), 1.93-1.87 (m, 1H), 0.91-0.85 (m, 2H), 0.68-0.63 (m, 2H).

### Example 102

### Synthesis of N-(2-acetyl-6-(((6-cyclopropylimidazo[1,2-a]pyridin-2-yl)methyl)amino)pyrimidin-4-yl)-2-(7-chloroimidazo[1,5-a]pyridin-1-yl)acetamide (I-102)

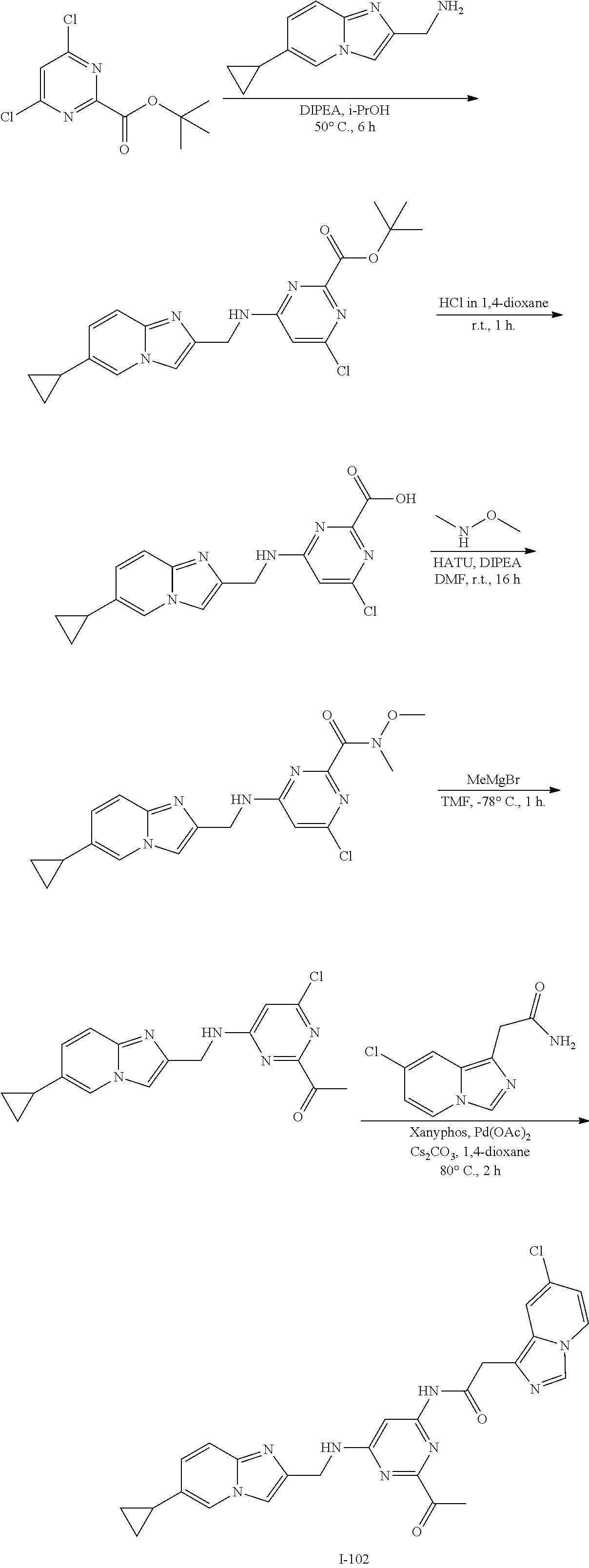

Synthesis of tert-butyl 4-chloro-6-(((6-cyclopropylimidazo[1,2-a]pyridin-2-yl)methyl)amino)pyrimidine-2-carboxylate. A mixture of tert-butyl 4,6-dichloropyrimidine-2-carboxylate (1.0 g, 4.0 mmol), (6-cyclopropylimidazo[1,2-a]pyridin-2-yl)methanamine (748 mg, 4.0 mmol) and DIPEA (1.0 g, 8.0 mmol) in i-PrOH (30 mL) was stirred at 50° C. for 6 h. The mixture was cooled to room temperature and concentrated to give the crude product, which was purified by column chromatography (eluent: DCM/MeOH=50/1) to give tert-butyl 4-chloro-6-(((6-cyclopropylimidazo[1,2-a]pyridin-2-yl)methyl)amino)pyrimidine-2-carboxylate (700 mg, yield: 44%) as a white solid. ESI-MS [M+H]+: 400.2.

Synthesis of 4-chloro-6-(((6-cyclopropylimidazo[1,2-a]pyridin-2-yl)methyl)amino)pyrimidine-2-carboxylic acid. A mixture of tert-butyl 4-chloro-6-(((6-cyclopropylimidazo[1,2-a]pyridin-2-yl)methyl)amino)pyrimidine-2-carboxylate (700 mg, 1.75 mmol) in HCl (4M solution in 1,4-dioxane, 10 mL) was stirred at room temperature for 1 h. The reaction mixture was concentrated to give 4-chloro-6-(((6-cyclopropylimidazo[1,2-a]pyridin-2-yl)methyl)amino)pyrimidine-2-carboxylic acid (600 mg, crude) as a yellow oil, which was used in the next step without purification. ESI-MS [M+H]+: 344.2.

Synthesis of 4-chloro-6-(((6-cyclopropylimidazo[1,2-a]pyridin-2-yl)methyl)amino)-N-methoxy-N-methylpyrimidine-2-carboxamide. A mixture of 4-chloro-6-(((6-cyclopropylimidazo[1,2-a]pyridin-2-yl)methyl)amino)pyrimidine-2-carboxylic acid (600 mg, crude), N,O-dimethylhydroxylamine (133 mg, 2.18 mmol), HATU (1.1 g, 2.9 mmol) and DIPEA (935 mg, 7.25 mmol) in DMF (10 mL) was stirred at room temperature for 16 h. The reaction was quenched with water (100 mL) and extracted with EtOAc (50 mL×3). The combined organic layers were washed with brine (50 mL), dried over anhydrous Na₂SO₄, and concentrated to give the crude product, which was purified by column chromatography (eluent: DCM/MeOH=30/1) to give 4-chloro-6-(((6-cyclopropylimidazo[1,2-a]pyridin-2-yl)methyl)amino)-N-methoxy-N-methylpyrimidine-2-carboxamide (450 mg, yield: 67% over 2 steps) as a white solid. ESI-MS [M+H]+: 387.2.

Synthesis of 1-(4-chloro-6-(((6-cyclopropylimidazo[1,2-a]pyridin-2-yl)methyl)amino)pyrimidin-2-yl)ethan-1-one. To a solution of 4-chloro-6-(((6-cyclopropylimidazo[1,2-a]pyridin-2-yl)methyl)amino)-N-methoxy-N-methylpyrimidine-2-carboxamide (390 mg, 1.0 mmol) in THF (10 mL) was added MeMgBr (3M solution in THF, 0.5 mL, 1.5 mmol) at −78° C. under N₂. After the reaction mixture was stirred at −78° C. for 2 h, the reaction was quenched with NH₄Cl (sat. aq., 50 mL) and extracted with EtOAc (30 mL×3). The combined organic layers were washed with brine (50 mL), dried over anhydrous Na₂SO₄, and concentrated to give the crude product, which was purified by column chromatography (eluent: DCM/MeOH=30/1) to give 1-(4-chloro-6-(((6-cyclopropylimidazo[1,2-a]pyridin-2-yl)methyl)amino)pyrimidin-2-yl)ethan-1-one (270 mg, yield: 79%) as a white solid. ESI-MS [M+H]+: 342.2.

Synthesis of N-(2-acetyl-6-(((6-cyclopropylimidazo[1,2-a]pyridin-2-yl)methyl)amino)pyrimidin-4-yl)-2-(7-chloroimidazo[1,5-a]pyridin-1-yl)acetamide. A mixture of 1-(4-chloro-6-(((6-cyclopropylimidazo[1,2-a]pyridin-2-yl)methyl)amino)pyrimidin-2-yl)ethan-1-one (150 mg, 0.44 mmol), 2-(7-chloroimidazo[1,5-a]pyridin-1-yl)acetamide (92 mg, 0.44 mmol), Pd(OAc)₂ (20 mg, 0.09 mmol), Xantphos (52 mg, 0.09 mmol) and Cs₂CO₃ (430 mg, 1.32 mmol) in 1,4-dioxane (8 mL) was stirred at 80° C. for 2 h under N₂. The reaction mixture was cooled to room temperature. The mixture was filtered through Celite® and the filter cake was washed with DCM/MeOH (10/1, 30 mL). The filtrate was concentrated to give the crude, which was purified by Prep-HPLC to give N-(2-acetyl-6-(((6-cyclopropylimidazo[1,2-a]pyridin-2-yl)methyl)amino)pyrimidin-4-yl)-2-(7-chloroimidazo[1,5-a]pyridin-1-yl)acetamide (45 mg, yield: 20%) as a white solid. ESI-MS [M+H]⁺: 515.2. ¹H NMR (400 MHz, DMSO) δ10.84 (s, 1H), 8.51 (s, 1H), 8.34-8.29 (m, 3H), 7.90 (s, 1H), 7.82 (s, 1H), 7.61 (d, J=8.4 Hz, 1H), 7.40 (s, 2H), 6.70-6.55 (m, 1H), 4.74 (s, 2H), 3.98 (s, 2H), 2.54 (s, 3H), 2.03-1.98 (m, 1H), 1.01-0.97 (m, 2H), 0.74-0.70 (m, 2H).

### Example 103

### Synthesis of 2-(7-chloroimidazo[1,5-a]pyridin-1-yl)-N-(6-(((6-cyclopropylimidazo[1,2-a]pyridin-2-yl)methyl)amino)-2-(1-hydroxyethyl)pyrimidin-4-yl)acetamide (I-103)

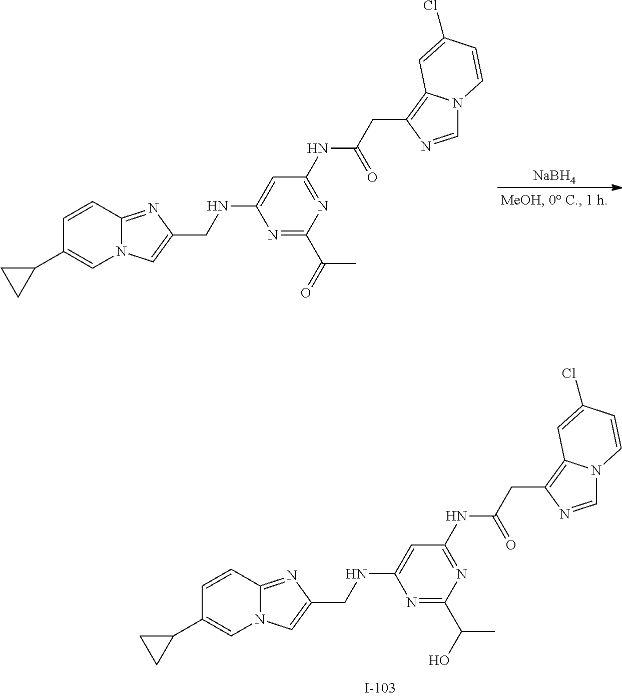 [SMILES: CC(=O)c1nc(NCc2cn3cc(C4CC4)ccc3n2)cc(NC(=O)Cc2ncn3ccc(Cl)cc23)n1 ; "NaBH4" ; CC(O)c1nc(NCc2cn3cc(C4CC4)ccc3n2)cc(NC(=O)Cc2ncn3ccc(Cl)cc23)n1]

To a solution of N-(2-acetyl-6-(((6-cyclopropylimidazo[1,2-a]pyridin-2-yl)methyl)amino)pyrimidin-4-yl)-2-(7-chloroimidazo[1,5-a]pyridin-1-yl)acetamide (25 mg, 0.05 mmol) (synthesis reported in Example 102) in MeOH (3 mL) was added NaBH₄ (10 mg, 0.25 mmol) at 0° C. After the reaction mixture was stirred at 0° C. for 1 h. the reaction was concentrated to give the crude product, which was purified by Prep-TLC (eluent: DCM/MeOH=20/1) to give 2-(7-chloroimidazo[1,5-a]pyridin-1-yl)-N-(6-(((6-cyclopropylimidazo[1,2-a]pyridin-2-yl)methyl)amino)-2-(1-hydroxyethyl)pyrimidin-4-yl)acetamide (10 mg, yield: 39%) as a white solid. ESI-MS [M+H]+: 517.2. ¹H NMR (400 MHz, DMSO) δ11.00 (s, 1H), 8.69 (s, 1H), 8.59-8.41 (m, 2H), 8.37 (d, J=7.4 Hz, 1H), 8.08 (s, 1H), 7.89 (s, 1H), 7.79 (d, J=9.3 Hz, 1H), 7.67 (d, J=9.3 Hz, 1H), 7.13 (s, 1H), 6.75 (d, J=6.5 Hz, 1H), 4.78 (s, 2H), 4.60-4.55 (m, 1H), 4.04 (s, 2H), 2.10-2.05 (m, 1H), 1.36 (d, J=6.6 Hz, 3H), 1.07-1.02 (m, 2H), 0.81-0.77 (m, 2H).

### Example 104

### Synthesis of 2-(6-chloroquinazolin-4-yl)-N-(6-(((6-cyclopropylimidazo[1,2-a]pyridin-2-yl)methyl)amino)pyrimidin-4-yl)acetamide (I-104)

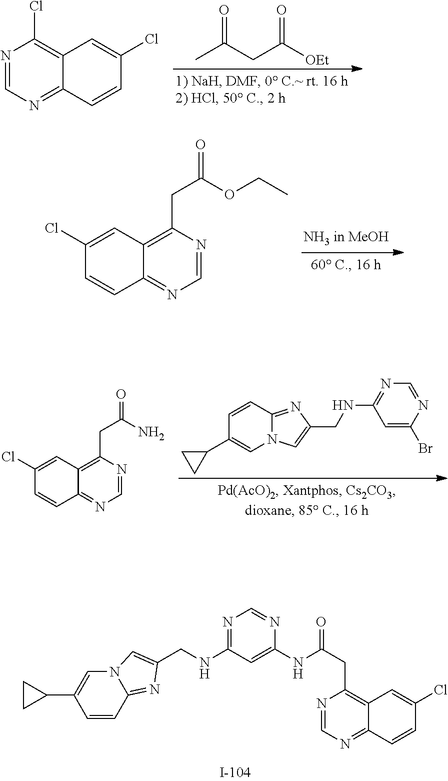

Synthesis of ethyl 2-(6-chloroquinazolin-4-yl)acetate. A mixture of ethyl 3-oxobutanoate (2.1 g, 16.1 mmol) and NaH (0.52 g, 13.0 mmol, 60% in mineral oil) in dry DMF (30 mL) was stirred at 0° C. for 2 h, then a solution of 4,6-dichloroquinazoline (2.0 g, 10.0 mmol) in DMF (2 mL) was added and the resulting mixture was stirred at room temperature for 16 h. HCl (1.5 M aq. 25 mL) was added and the mixture was stirred at 50° C. for 2 h. The reaction was diluted with water (300 mL) and extracted with EtOAc (100 mL×3). The combined organic layers were washed with brine (100 mL), dried over Na₂SO₄ and concentrated. The residue was purified by column chromatography (eluent: EtOAc/PE=0-35%) to give ethyl 2-(6-chloroquinazolin-4-yl)acetate (1.0 g, yield: 40%) as a yellow oil. ESI-MS [M+H]⁺: 251.0.

Synthesis of 2-(6-chloroquinazolin-4-yl)acetamide. A solution of ethyl 2-(6-chloroquinazolin-4-yl)acetate (1.0 g, 4.0 mmol) in dry NH₃ (100 mL, 7 M in MeOH) was stirred in a sealed tube at 60° C. for 16 h. After cooling to room temperature, the reaction mixture was concentrated and purified by column chromatography (eluent: MeOH/DCM=0-10%) to give 2-(6-chloroquinazolin-4-yl)acetamide (500 mg, yield: 57%) as a yellow solid. ESI-MS [M+H]⁺: 222.1.

Synthesis of 2-(6-chloroquinazolin-4-yl)-N-(6-(((6-cyclopropylimidazo[1,2-a]pyridin-2-yl)methyl)amino)pyrimidin-4-yl)acetamide. A mixture of 2-(6-chloroquinazolin-4-yl)acetamide (50 mg, 0.22 mmol), 6-bromo-N-((6-cyclopropylimidazo[1,2-a]pyridin-2-yl)methyl)pyrimidin-4-amine (78 mg, 0.22 mmol) (synthesis reported in Example 99), Pd(OAc)₂ (10 mg, 0.044 mmol), Xantphos (25 mg, 0.044 mmol) and Cs₂CO₃ (216 mg, 0.66 mmol) in 1.4-Dioxane (5 mL) was stirred at 85° C. for 16 h under N₂. After cooling to room temperature, the reaction mixture was filtered through Celite® and the filter cake was washed with DCM/MeOH (10/1, 30 mL). The filtrate was concentrated to give the crude, which was purified by Prep-HPLC to give 2-(6-chloroquinazolin-4-yl)-N-(6-(((6-cyclopropylimidazo[1,2-a]pyridin-2-yl)methyl)amino)pyrimidin-4-yl)acetamide (5.0 mg, yield: 5%) as a yellow solid. ESI-MS [M+H]⁺: 485.1. ¹H NMR (400 MHz, DMSO) δ12.56 (s, 1H), 9.88 (s, 1H), 8.41 (s, 1H), 8.30 (s, 1H), 8.19 (s, 1H), 7.97 (s, 1H), 7.81 (s, 1H), 7.67 (s, 2H), 7.62 (s, 1H), 7.45 (d, J=9.0 Hz, 1H), 7.37 (d, J=9.2 Hz, 1H), 7.30 (s, 1H), 6.96 (d, J=8.1 Hz, 1H), 6.04 (s, 1H), 4.57 (s, 2H), 3.60 (s, 2H), 1.92-1.88 (m, 1H), 0.91-0.89 (m, 2H), 0.67-0.63 (m, 2H).

### Example 105

### Synthesis of 2-(7-chlorophthalazin-1-yl)-N-(6-(((6-cyclopropylimidazo[1,2-a]pyridin-2-yl)methyl)amino)pyrimidin-4-yl)acetamide (I-105)

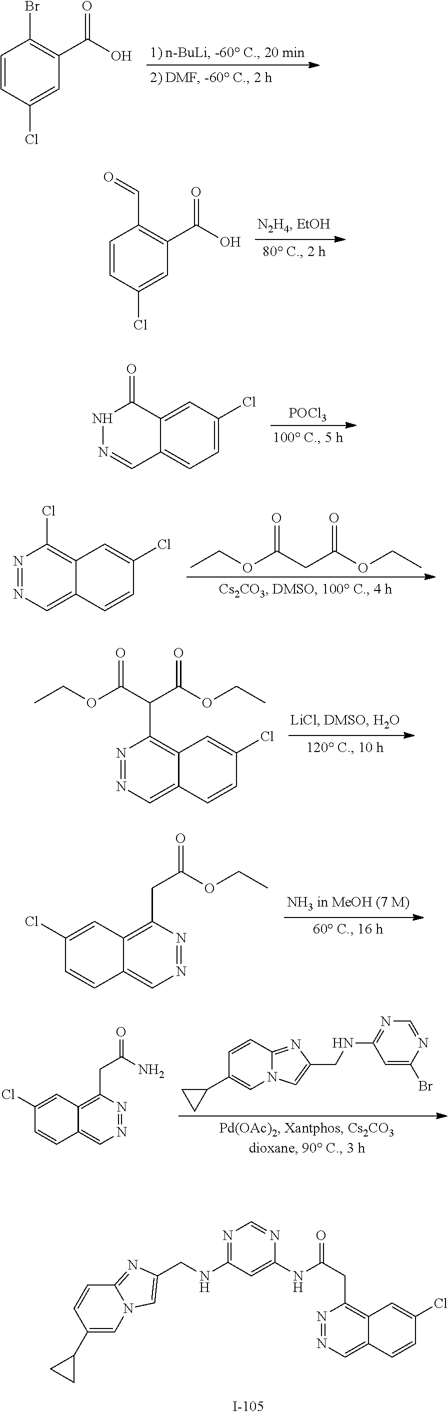

Synthesis of 5-chloro-2-formylbenzoic acid. To a solution of 2-bromo-5-chlorobenzoic acid (8 g, 34 mmol) in THF (150 mL) was added n-BuLi (75 mmol, 31 mL, 2.4 M in THF) dropwise at −60° C. and the mixture was stirred at −60° C. for 30 min. DMF (4.97 g, 68 mmol) was added dropwise and the mixture was stirred at −60° C. for another 2 h. Then reaction mixture was quenched with NH₄Cl (sat. aq., 100 mL), stirred and warmed to room temperature, extracted with EtOAc (100 mL×1). The organic layer was discarded. The aqueous layer was acidified to pH=4˜5 with HCl (2 M, aq.) and extracted with EtOAc (100 mL×3). The combined organics was washed with brine (100 mL), dried over Na₂SO₄, concentrated to give 5-chloro-2-formylbenzoic acid (3.14 g, yield: 50%) as a light brown solid. ESI-MS [M+H]+: 185.0.

Synthesis of 7-chlorophthalazin-1 (2H)-one. A mixture of 5-chloro-2-formylbenzoic acid (3.14 g, 17 mmol) and hydrazine hydrate (4.26 g, 85 mmol) in EtOH (30 mL) was stirred at 80° C. for 2 h. After cooling to room temperature, the reaction mixture was concentrated. The residue was diluted with water (100 mL) and filtered. The solid was dried in vacuo to afford 7-chlorophthalazin-1 (2H)-one (2.7 g, 88%) as a white solid. ESI-MS [M+H]+: 181.0.

Synthesis of 1,7-dichlorophthalazine. A mixture of 7-chlorophthalazin-1 (2H)-one (2.7 g, 15 mmol) and POCl₃ (10 mL) was stirred at 100° C. for 5 h. After cooling to room temperature, the reaction mixture was concentrated and diluted with EtOAc (150 mL). The resulting solution was washed with NaHCO₃ (sat. aq., 100 mL) and brine (100 mL), dried over Na₂SO₄ and concentrated to give the crude, which was purified by column chromatography (eluent: EtOAc/PE=0-35%) to afford 1,7-dichloropthalazine (1.43 g, 48%) as a yellow solid. ESI-MS [M+H]+: 199.0.

Synthesis of diethyl 2-(7-chlorophthalazin-1-yl)malonate. A mixture of 1,7-dichlorophthalazine (1.43 g, 7.2 mmol), diethyl malonate (1.73 g, 10.8 mmol) and Cs₂CO₃ (4.71 g, 14.4 mmol) in DMSO (30 mL) was stirred at 100° C. for 4 h. After cooling to room temperature, the reaction mixture was poured into water (300 mL) and extracted with EtOAc (50 mL×3). The combined organics was washed with water (50 mL) and brine (50 mL), dried over Na₂SO₄, concentrated and dried in vacuo to give diethyl 2-(7-chlorophthalazin-1-yl)malonate (2.32 g, yield: quant) as a yellow solid, which was used in the next step without purification. ESI-MS [M+H]+: 323.0.

Synthesis of ethyl 2-(7-chlorophthalazin-1-yl)acetate. A mixture of 2-(7-chlorophthalazin-1-yl)malonate (2.32 g, 7.2 mmol) and LiCl (610 mg, 14.4 mmol) in DMSO (20 mL) and H₂O (1 mL) was stirred at 120° C. for 10 h. After cooling to room temperature, the reaction mixture was poured into water (200 mL) and extracted with EtOAc (80 mL×3). The combined organics was washed with brine (50 mL), dried over Na₂SO₄, concentrated to give the crude, which was purified by column chromatography (eluent: EtOAc/PE=0-50%) to give ethyl 2-(7-chlorophthalazin-1-yl)acetate (1.0 g, yield: 56%) as a yellow solid. ESI-MS [M+H]+: 251.0.

Synthesis of 2-(7-chlorophthalazin-1-yl)acetamide. A mixture of ethyl 2-(7-chlorophthalazin-1-yl)acetate (220 mg, 0.88 mmol) and NH₃/MeOH (10 mL, 7 M) was stirred in a sealed tube at 60° C. for 16 h. After cooling to room temperature, the reaction mixture was concentrated and purified by chromatography (eluent: MeOH/DCM=0-5%) to afford 2-(7-chlorophthalazin-1-yl)acetamide (180 mg, yield: 93%) as a yellow solid. ESI-MS [M+H]+: 222.1.

Synthesis of 2-(7-chlorophthalazin-1-yl)-N-(6-(((6-cyclopropylimidazo[1,2-a]pyridin-2-yl)methyl)amino)pyrimidin-4-yl)acetamide. A mixture of 6-bromo-N-((6-cyclopropylimidazo[1,2-a]pyridin-2-yl)methyl)pyrimidin-4-amine (217 mg, 0.63 mmol), 2-(7-chlorophthalazin-1-yl)acetamide (140 mg, 0.63 mmol), Pd(OAc)₂ (14 mg, 0.063 mmol), Xantphos (75 mg, 0.13 mmol) and Cs₂CO₃ (621 mg, 1.9 mmol) in 1,4-dioxane (10 mL) was stirred in sealed tube at 90° C. for 3 h under N₂. After cooling to room temperature, the reaction mixture was filtered through Celite® and the filter cake was washed with DCM/MeOH (10/1, 50 mL). The filtrate was concentrated to give the crude, which was purified by column chromatography (eluent: DCM/MeOH=0-5%) and Prep-HPLC to give 2-(7-chlorophthalazin-1-yl)-N-(6-(((6-cyclopropylimidazo[1,2-a]pyridin-2-yl)methyl)amino)pyrimidin-4-yl)acetamide (100 mg, yield: 33%) as a yellow solid. ESI-MS [M+H]+: 485.1. ¹H NMR (400 MHz, DMSO) δ9.73-9.64 (m, 1H), 8.50-8.03 (m, 4H), 7.94-7.73 (m, 2H), 7.61 (d, J=17.9 Hz, 1H), 7.40-7.20 (m, 2H), 6.98-6.93 (m, 1H), 5.87 (s, 1H), 4.57 (s, 4H), 1.95-1.85 (m, 1H), 0.97-0.83 (m, 2H), 0.69-0.62 (m, 2H).

### Example 106

### Synthesis of 2-(7-chloro-8-fluoroimidazo[1,5-a]pyridin-1-yl)-N-(6-(((6-cyclopropylimidazo[1,2-a]pyridin-2-yl)methyl)amino)pyrimidin-4-yl)-2-hydroxypropanamide (I-106)

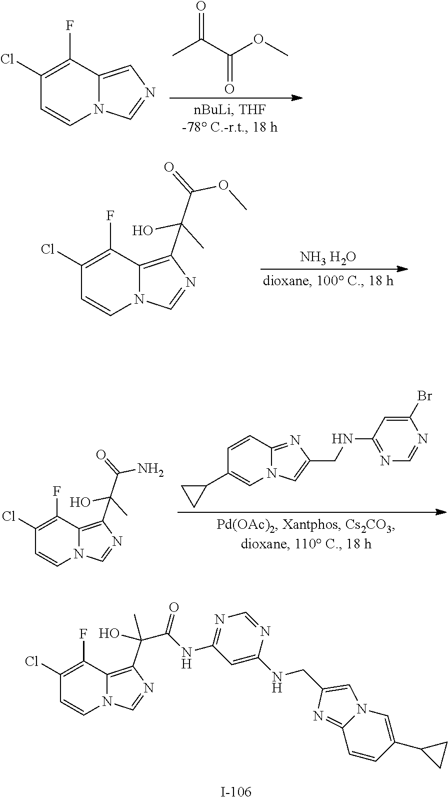

Synthesis of methyl 2-(7-chloro-8-fluoroimidazo[1,5-a]pyridin-1-yl)-2-hydroxypropanoate. To a solution of Methyl 2-oxopropanoate (398 mg, 3.9 mmol) in THF (20 mL) was added n-butyllithium (1.7 mL, 4.1 mmol, 2.4M in THF) at −78° C. and the mixture was stirred this temperature for 30 min. 7-chloro-8-fluoroimidazo[1,5-a]pyridine (566 mg, 3.3 mmol) in THF (5 mL) was added and the resulting mixture was warmed to room temperature and stirred for 18 h. The reaction was then quenched with saturated aqueous NH₄Cl (sat. aq., 50 mL) and extracted with EtOAc (30 mL×3). The combined organic layers were washed with brine (30 mL), dried over Na₂SO₄, and concentrated to give the crude product, which was purified by column chromatography (eluent: EtOAc/PE=0-20%) to give methyl 2-(7-chloro-8-fluoroimidazo[1,5-a]pyridin-1-yl)-2-hydroxypropanoate (170 mg, yield: 19%) as an pale yellow solid. ESI-MS [M+H]⁺:273.0.

Synthesis of 2-(7-chloro-8-fluoroimidazo[1,5-a]pyridin-1-yl)-2-hydroxypropanamide. To a solution of methyl 2-(7-chloro-8-fluoroimidazo[1,5-a]pyridin-1-yl)-2-hydroxypropanoate) (170 mg, 0.6 mmol) in dioxane (5 mL) in a sealed tube was added ammonium hydroxide (3 mL) and the reaction was stirred at 100° C. for 18 h. After cooling to room temperature, the mixture was concentrated to give the crude, which was purified by column chromatography (eluent: MeOH/DCM=0-10%) to give 2-(7-chloro-8-fluoroimidazo[1,5-a]pyridin-1-yl)-2-hydroxypropanamide (140 mg, yield: 91%) as an pale yellow solid. ESI-MS [M+H]⁺:258.0.

Synthesis of 2-(7-chloro-8-fluoroimidazo[1,5-a]pyridin-1-yl)-N-(6-(((6-cyclopropylimidazo[1,2-a]pyridin-2-yl)methyl)amino)pyrimidin-4-yl)-2-hydroxypropanamide. A mixture of 2-(7-chloro-8-fluoroimidazo[1,5-a]pyridin-1-yl)-2-hydroxypropanamide (42 mg, 0.16 mmol), 6-bromo-N-((6-cyclopropylimidazo[1,2-a]pyridin-2-yl)methyl)pyrimidin-4-amine (72 mg, 0.21 mmol), Pd(OAc)₂ (7 mg 0.03 mmol), Xantphos (17 mg, 0.03 mmol) and Cs₂CO₃ (156 mg 0.48 mmol) in dioxane (10 mL) was stirred at 110° C. under N₂ for 18 h. After cooling to room temperature, the reaction was quenched with water (50 mL) and extracted with EtOAc (50 mL×3). The combined organic layers were washed with brine (50 mL), dried over Na₂SO₄, concentrated to give the crude, which was purified by column chromatography (eluent: MeOH/DCM=0-10%) to give 2-(7-chloro-8-fluoroimidazo[1,5-a]pyridin-1-yl)-N-(6-(((6-cyclopropylimidazo[1,2-a]pyridin-2-yl)methyl)amino)pyrimidin-4-yl)-2-hydroxypropanamide (8.5 mg, yield: 10%) as an off white solid. ESI-MS [M+H]+: 520.5. ¹H NMR (400 MHz, DMSO) δ9.60 (s, 1H), 8.30-8.26 (m, 2H), 8.07 (d, J=7.6 Hz, 1H), 7.99 (s, 1H), 7.60 (d, J=9.3 Hz, 2H), 7.39-7.34 (m, 2H), 7.19 (s, 1H), 6.95 (dd, J=9.3, 1.8 Hz, 1H), 6.82 (t, J=7.1 Hz, 1H), 4.59 (s, 2H), 1.97 (s, 3H), 1.93-1.87 (m, 1H), 0.94-0.89 (m, 2H), 0.68-0.63 (m, 2H).

### Example 107

### Synthesis of N-((6-amino-2,4-dimethylpyridin-3-yl)methyl)-6-((6-cyclopropylimidazo[1,2-a]pyridin-2-yl)methoxy)pyrimidin-4-amine (I-107)

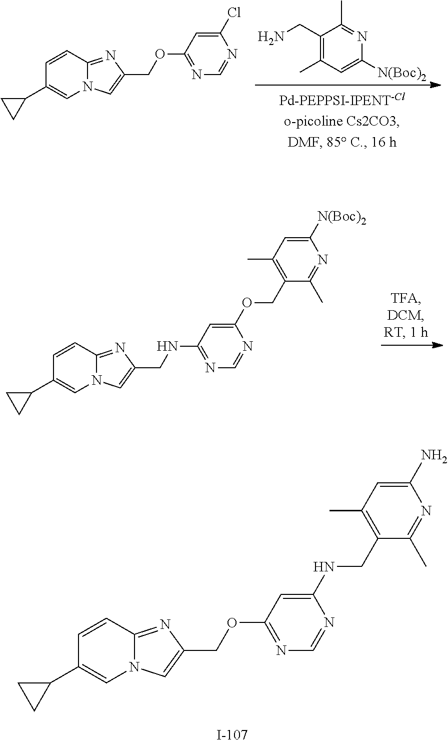

Synthesis of D-tert-butyl (5-(((6-(((6-cyclopropylimidazo[1,2-a]pyridin-2-yl)methyl)amino)pyrimidin-4-yl)oxy)methyl)-4,6-dimethylpyridin-2-yl)carbamate. To a mixture of 2-(((6-chloropyrimidin-4-yl)oxy)methyl)-6-cyclopropylimidazo[1,2-a]pyridine (60 mg, 0.2 mmol) (synthesis reported in Example 72) and tert-butyl (5-(aminomethyl)-4,6-dimethylpyridin-2-yl)(tert-butoxycarbonyl)carbamate (105 mg, 0.3 mmol) in DMF (5 mL) was added Cs₂CO₃ (196 mg, 0.6 mmol) and Pd-PEPPSI-IPENT-^{Cl} o-picoline (17 mg, 0.02 mmol), the resulting reaction was stirred at 85° C. for 16 h under N₂. After cooling to room temperature, the reaction mixture was filtered through Celite® and the filter cake was washed with DCM/MeOH (10/1, 30 mL). The filtrate was concentrated to give the crude, which was purified by Prep-TLC (eluent: DCM/MeOH=10/1) to give tert-butyl (tert-butoxycarbonyl)(5-(((6-(((6-cyclopropylimidazo[1,2-a]pyridin-2-yl)methyl)amino)pyrimidin-4-yl)oxy)methyl)-4,6-dimethylpyridin-2-yl)carbamate (110 mg, yield: 89%) as a white solid. ESI-MS [M+H]⁺: 616.3.

Synthesis of N-((6-amino-2,4-dimethylpyridin-3-yl)methyl)-6-((6-cyclopropylimidazo[1,2-a]pyridin-2-yl)methoxy)pyrimidin-4-amine. To a solution of tert-butyl (5-(((6-(((6-cyclopropylimidazo[1,2-a]pyridin-2-yl)methyl)amino)pyrimidin-4-yl)oxy)methyl)-4,6-dimethylpyridin-2-yl)carbamate (110 mg, 0.18 mmol) in dry DCM (5 mL) was added TEA (1 ml) at 0° C. and the resulting reaction was stirred at room temperature for 1 h. The reaction was quenched with NaHCO₃ (sat. aq., 50 mL) and extracted with EtOAc (50 mL×3). The combined organic layers were washed with brine (50 mL), dried over Na₂SO₄, concentrated to give the crude, which was purified by Prep-TLC (eluent: DCM/MeOH=10/1) to give the product as white solid (40 mg, yield: 53%). ESI-MS [M+H]+: 416.2. ¹H NMR (400 MHz, DMSO) δ8.33 (s, 1H), 8.24 (s, 1H), 7.81 (s, 1H), 7.41 (d, J=9.3 Hz, 1H), 7.05 (s, 1H), 7.02-6.97 (m, 1H), 6.13 (s, 1H), 5.84 (s, 1H), 5.67 (s, 2H), 5.35 (s, 2H), 4.29 (s, 2H), 2.28 (s, 3H), 2.14 (s, 3H), 1.97-1.86 (m, 1H), 0.96-0.90 (m, 2H), 0.71-0.65 (m, 2H).

### Example 108

### Synthesis of N-(4-(aminomethyl)-2,6-dimethylbenzyl)-6-((6-cyclopropylimidazo[1,2-a]pyridin-2-yl)methoxy)pyrimidin-4-amine (I-108)

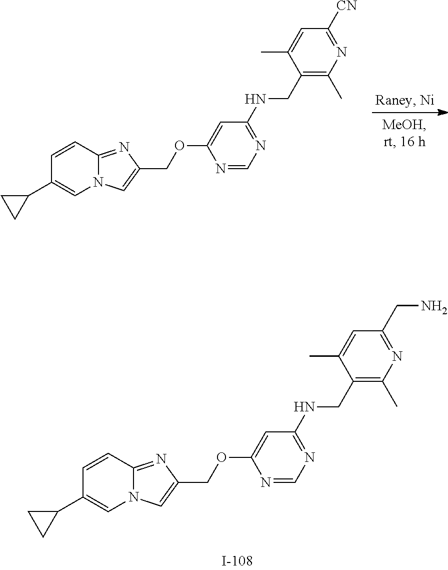 [SMILES: Cc1cc(C#N)nc(C)c1CNc1cc(OCc2cn3cc(C4CC4)ccc3n2)ncn1 ; Cc1cc(CN)nc(C)c1CNc1cc(OCc2cn3cc(C4CC4)ccc3n2)ncn1]

A mixture of 4-(((6-((6-cyclopropylimidazo[1,2-a]pyridin-2-yl)methoxy)pyrimidin-4-yl)amino)methyl)-3,5-dimethylbenzonitrile (50 mg, 0.12 mmol) (synthesis reported in Example 72) and Raney Ni (30 mg) in MeOH (10 ml) was stirred at room temperature for 16 h under H₂. The mixture was filtered and the filter cake was washed with MeOH (20 mL), the filtrate was concentrated to give the crude product, which was purified by Prep-TLC (eluent: DCM/MeOH=10/1) to give N-(4-(aminomethyl)-2,6-dimethylbenzyl)-6-((6-cyclopropylimidazo[1,2-a]pyridin-2-yl)methoxy)pyrimidin-4-amine (11 mg, yield: 22%) as a white solid. ESI-MS [M+H]⁺: 429.2. ¹H NMR (400 MHz, DMSO) δ 8.33 (s, 1H), 8.25 (s, 1H), 7.80 (s, 1H), 7.40 (d, J=9.3 Hz, 1H), 7.12-7.09 (m, 1H), 6.99-6.88 (m, 3H), 5.85 (s, 1H), 5.34 (s, 2H), 4.46-4.38 (m, 2H), 3.63 (s, 2H), 3.25-2.96 (m, 2H), 2.30 (s, 6H), 1.97-1.88 (m, 1H), 0.96-0.88 (m, 2H), 0.71-0.61 (m, 2H).

### Example 109

### Synthesis of N-((6-amino-2,4-dimethylpyridin-3-yl)methyl)-6-((6-cyclopropyl-8-(3-fluorooxetan-3-yl)imidazo[1,2-a]pyridin-2-yl)methoxy)pyrimidin-4-amine (I-109)

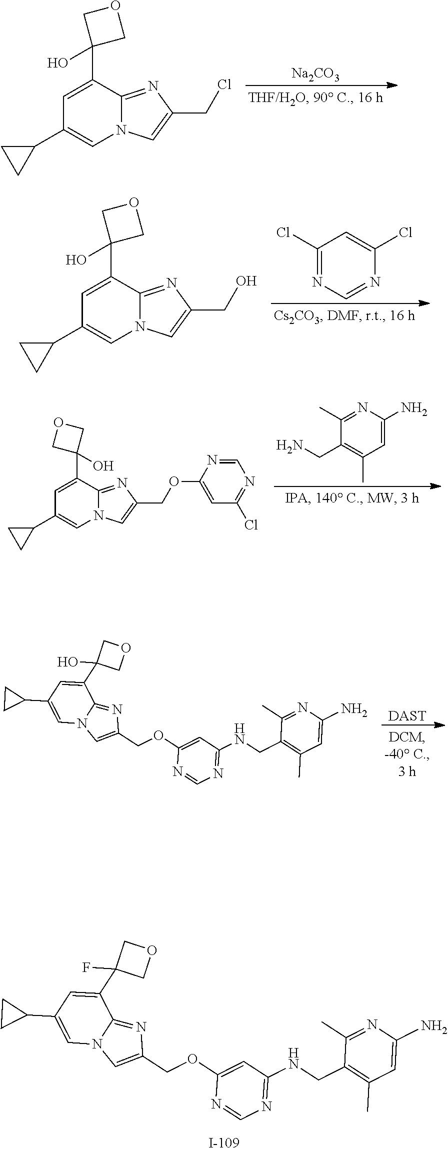

Synthesis of 3-(6-cyclopropyl-2-(hydroxymethyl)imidazo[1,2-a]pyridin-8-yl)oxetan-3-ol. A mixture of 3-(2-(chloromethyl)-6-cyclopropylimidazo[1,2-a]pyridin-8-yl)oxetan-3-ol (200 mg, 0.72 mmol) and Na₂CO₃ (229 mg, 2.16 mmol) in THF/H₂O (10 ml/10 ml) was stirred at 90° C. for 16 h. After cooling to room temperature, the reaction was quenched with water (50 mL) and extracted with EtOAc (50 mL×3). The combined organic layers were washed with brine (50 mL), dried over Na₂SO₄, concentrated to give the crude, which was purified by column chromatography (eluent: MeOH/DCM=0-5%) to give the 3-(6-cyclopropyl-2-(hydroxymethyl)imidazo[1,2-a]pyridin-8-yl)oxetan-3-ol (70 mg, yield: 37%) as a yellow solid. ESI-MS [M+H]⁺: 261.2.

Synthesis of 3-(2-(((6-chloropyrimidin-4-yl)oxy)methyl)-6-cyclopropylimidazo[1,2-a]pyridin-8-yl)oxetan-3-ol. A mixture of 3-(6-cyclopropyl-2-(hydroxymethyl)imidazo[1,2-a]pyridin-8-yl)oxetan-3-ol (52 mg, 0.2 mmol), 4,6-dichloropyrimidine (37 mg, 0.25 mmol) and Cs₂CO₃ (164 mg, 0.5 mmol) in DMF (5 mL) was stirred at room temperature for 16 h. The reaction was quenched with water (50 mL) and extracted with EtOAc (50 mL×3). The combined organic layers were washed with brine (50 mL), dried over Na₂SO₄, concentrated to give the crude, which was purified by Prep-TLC (eluent: DCM/MeOH=10/1) to give the 3-(2-(((6-chloropyrimidin-4-yl)oxy)methyl)-6-cyclopropyl imidazo[1,2-a]pyridin-8-yl)oxetan-3-ol (40 mg, yield: 54%) as a yellow solid. ESI-MS [M+H]⁺: 373.2.

Synthesis of 3-(2-(((6-(((6-amino-2,4-dimethylpyridin-3-yl)methyl)amino)pyrimidin-4-yl)oxy)methyl)-6-cyclopropylimidazo[1,2-a]pyridin-8-yl)oxetan-3-ol. A mixture of 3-(2-(((6-chloropyrimidin-4-yl)oxy)methyl)-6-cyclopropylimidazo[1,2-a]pyridin-8-yl)oxetan-3-ol (20 mg, 0.054 mmol), 5-(aminomethyl)-4,6-dimethylpyridin-2-amine (12 mg, 0.081 mmol) and DIPEA (21 mg, 0.16 mol) in i-PrOH (2 mL) was stirred in a sealed tube. After degassing with N₂ for 1 min, the reaction was irradiated in microwave at 140° C. for 3 h. The reaction was concentrated to give the crude product, which was purified by Prep-TLC (eluent: DCM/MeOH=10/1) to give the 3-(2-(((6-(((6-amino-2,4-dimethylpyridin-3-yl)methyl)amino)pyrimidin-4-yl)oxy)methyl)-6-cyclopropylimidazo[1,2-a]pyridin-8-yl)oxetan-3-ol (15 mg, yield: 57%) as a yellow solid. ESI-MS [M+H]⁺: 488.2.

Synthesis of N-((6-amino-2,4-dimethylpyridin-3-yl)methyl)-6-((6-cyclopropyl-8-(3-fluorooxetan-3-yl)imidazo[1,2-a]pyridin-2-yl)methoxy)pyrimidin-4-amine. To a solution of 3-(2-(((6-(((6-amino-2,4-dimethylpyridin-3-yl)methyl)amino)pyrimidin-4-yl)oxy)methyl)-6-cyclopropylimidazo[1,2-a]pyridin-8-yl)oxetan-3-ol (15 mg, 0.031 mmol) in DCM (5 mL) was added DAST (25 mg, 0.16 mmol) at −40° C. under nitrogen. The mixture was stirred at −40° C. for 3 h. The reaction was quenched with water (20 mL) and extracted with EtOAc (30 mL×3). The combined organic layers were washed with brine (20 mL), dried over Na₂SO₄, concentrated to give the crude, which was purified by Prep-TLC (eluent: DCM/MeOH=10/1) to give the N-((6-amino-2,4-dimethylpyridin-3-yl)methyl)-6-((6-cyclopropyl-8-(3-fluorooxetan-3-yl)imidazo[1,2-a]pyridin-2-yl)methoxy)pyrimidin-4-amine (5 mg, yield: 33%) as a white solid. ESI-MS [M+H]⁺: 490.2. 1H NMR (400 MHz, DMSO) δ8.42 (s, 1H), 8.24 (s, 1H), 7.88 (s, 1H), 7.15 (s, 1H), 7.07 (s, 1H), 6.15 (s, 1H), 5.85 (s, 1H), 5.78 (s, 2H), 5.41-5.32 (m, 3H), 5.30 (d, J=8.8 Hz, 1H), 5.00 (d, J=8.7 Hz, 1H), 4.94 (d, J=8.8 Hz, 1H), 4.29 (s, 2H), 2.29 (s, 3H), 2.15 (s, 3H), 1.98-1.92 (m, 1H), 0.97-0.88 (m, 2H), 0.77-0.70 (m, 2H).

### Example 110

### Synthesis of 5-(((4-((6-ethylimidazo[1,2-a]pyridin-2-yl)methoxy)pyridin-2-yl)amino)methyl)-4,6-dimethylpyridin-2-amine (I-110)

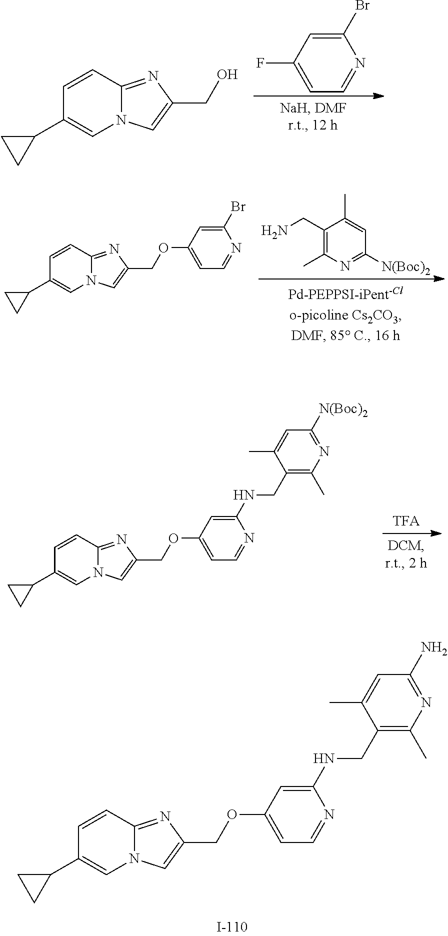

Synthesis of 2-(((2-bromopyridin-4-yl)oxy)methyl)-6-cyclopropylimidazo[1,2-a]pyridine. To a mixture of (6-cyclopropylimidazo[1,2-a]pyridin-2-yl)methanol (50 mg, 0.27 mmol) in DMF (4 mL) was added NaH (22 mg, 0.54 mmol, 60% in mineral oil) and 2-bromo-4-fluoropyridine (56 mg, 0.32 mmol), the resulting mixture was stirred at room temperature for 12 h. The reaction was quenched with NH₄Cl (sat. aq., 30 mL) and extracted with EtOAc (30 mL×3). The combined organic layers were washed with brine (30 mL), dried over Na₂SO₄, concentrated to give the crude, which was purified by Prep-TLC (eluent: DCM/MeOH=20/1) to give 2-(((2-bromopyridin-4-yl)oxy)methyl)-6-cyclopropylimidazo[1,2-a]pyridine (50 mg, yield: 54%) as a white solid. ESI-MS [M+H]+: 344.1.

Synthesis of N-((6-(bis(pivaloyloxy)amino)-2,4-dimethylpyridin-3-yl)methyl)-4-((6-cyclopropylimidazo[1,2-a]pyridin-2-yl)methoxy)pyridin-2-amine compound. To a mixture of 2-(((2-bromopyridin-4-yl)oxy)methyl)-6-cyclopropylimidazo[1,2-a]pyridine (50 mg, 0.14 mmol) and (6-(bis(pivaloyloxy)amino)-2,4-dimethylpyridin-3-yl)methanamine (66 mg, 0.19 mmol) in DMF (4 mL) was added Pd-PEPSI-Ipent-Cl, D-pidine (17 mg, 0.02 mmol) and Cs₂CO₃ (85 mg, 0.26 mmol). The mixture was stirred at 85° C. for 16 h under N₂. After cooling to room temperature, the reaction mixture was filtered through Celite® and the filter cake was washed with DCM/MeOH (10/1, 30 mL). The filtrate was concentrated to give the crude, which was purified by Prep-TLC (eluent: DCM/MeOH=20/1) to give N-((6-(bis(pivaloyloxy)amino)-2,4-dimethylpyridin-3-yl)methyl)-4-((6-cyclopropylimidazo[1,2-a]pyridin-2-yl)methoxy)pyridin-2-amine (40 mg, yield: 46%) as a white solid. ESI-MS [M+H]+: 615.1.

Synthesis of 5-(((4-((6-cyclopropylimidazo[1,2-a]pyridin-2-yl)methoxy)pyridin-2-yl)amino)methyl)-4,6-dimethylpyridin-2-amine. To a solution of N-((6-(bis(pivaloyloxy)amino)-2,4-dimethylpyridin-3-yl)methyl)-4-((6-cyclopropylimidazo[1,2-a]pyridin-2-yl)methoxy)pyridin-2-amine (40 mg, 0.065 mmol) in DCM (4 mL) was added TFA (1 mL) and the mixture was stirred at room temperature for 2 h. The reaction was quenched with NaHCO₃ (sat. aq., 20 mL) and extracted with EtOAc (30 mL×3). The combined organic layers were washed with brine (30 mL), dried over Na₂SO₄, concentrated to give the crude, which was purified by Prep-TLC (eluent: DCM/MeOH=10/1) to give 5-(((4-((6-cyclopropylimidazo[1,2-a]pyridin-2-yl)methoxy)pyridin-2-yl)amino)methyl)-4,6-dimethylpyridin-2-amine (10 mg, yield: 37%) as a white solid. ESI-MS [M+H]⁺: 415.3. ¹H NMR (400 MHz, DMSO) δ8.34 (s, 1H), 7.86-7.78 (m, 2H), 7.41 (d, J=9.3, 1H), 7.01-6.88 (m, 1H), 6.23-6.20 (m, 2H), 6.18-6.08 (m, 2H), 5.66 (s, 2H), 5.10 (s, 2H), 4.24 (d, J=4.3, 2H), 2.29 (s, 3H), 2.15 (s, 3H), 1.98-1.87 (m, 1H), 0.95-0.88 (m, 2H), 0.73-0.64 (m, 2H).

### Example 111

### Synthesis of N⁴-((6-amino-2,4-dimethylpyridin-3-yl)methyl)-N⁶-((6-cyclopropylimidazo[1,2-a]pyridin-2-yl)methyl)pyrimidine-4,6-diamine (I-111)

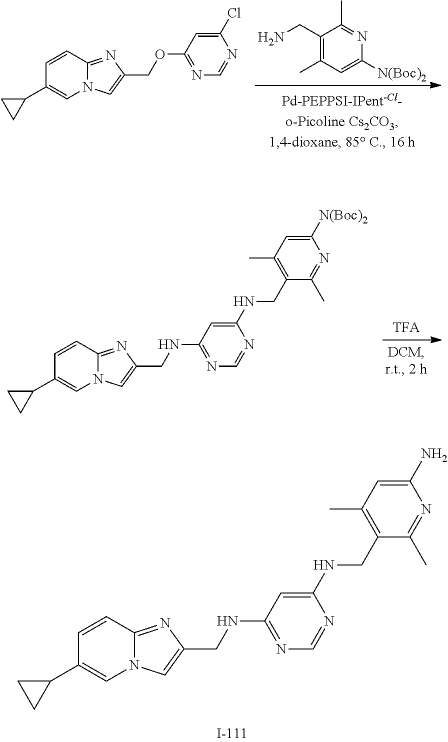

Synthesis of tert-butyl (tert-butoxycarbonyl)(5-(((6-(((6-cyclopropylimidazo[1,2-a]pyridin-2-yl)methyl)amino)pyrimidin-4-yl)amino)methyl)-4,6-dimethylpyridin-2-yl)carbamate. A mixture of 6-chloro-N-((6-cyclopropylimidazo[1,2-a]pyridin-2-yl)methyl)pyrimidin-4-amine (120 mg, 0.4 mmol), tert-butyl (5-(aminomethyl)-4,6-dimethylpyridin-2-yl)(tert-butoxycarbonyl)carbamate (211 mg, 0.6 mmol), Pd-PEPPSI-IPent-^{Cl}-o-Picoline (34 mg, 0.04 mmol) and Cs₂CO₃ (391 mg, 1.2 mmol) in 1,4-dioxane (5 mL) was stirred at 85° C. for 16 h under N₂. The reaction mixture was cooled to room temperature then filtered through Celite® and the filter cake was washed with DCM/MeOH (10/1, 30 mL). The filtrate was concentrated to give the crude, which was purified by Prep-TLC (eluent: DCM/MeOH=10/1) to give tert-butyl (tert-butoxycarbonyl)(5-(((6-(((6-cyclopropylimidazo[1,2-a]pyridin-2-yl)methyl)amino)pyrimidin-4-yl)amino)methyl)-4,6-dimethylpyridin-2-yl)carbamate (80 mg, yield: 33%) as a white solid. ESI-MS [M+H]⁺: 615.2.

Synthesis of N⁴-((6-amino-2,4-dimethylpyridin-3-yl)methyl)-N⁶-((6-cyclopropylimidazo[1,2-a]pyridin-2-yl)methyl)pyrimidine-4,6-diamine. To a solution of tert-butyl (tert-butoxycarbonyl)(5-(((6-(((6-cyclopropylimidazo[1,2-a]pyridin-2-yl)methyl)amino)pyrimidin-4-yl)amino)methyl)-4,6-dimethylpyridin-2-yl)carbamate (80 mg, 0.13 mmol) in DCM (5 mL) was added TFA (1 mL) at 0° C. After the reaction mixture was stirred at room temperature for 1 h, the reaction was quenched with NaHCO₃ (sat. aq., 30 mL), extracted with DCM/MeOH (10/1, 20 mL×3). The combined organic layers were washed with brine (30 mL), dried over Na₂SO₄ and concentrated to give the crude, which was purified by Prep-TLC (eluent: DCM/MeOH=10/1) to give N⁴-((6-amino-2,4-dimethylpyridin-3-yl)methyl)-N⁶-((6-cyclopropylimidazo[1,2-a]pyridin-2-yl)methyl)pyrimidine-4,6-diaminet (43 mg, yield: 80%) as a white solid. ESI-MS [M+H]⁺: 415.2. ¹H NMR (400 MHz, DMSO) δ 8.29 (s, 1H), 7.96 (s, 1H), 7.58 (s, 1H), 7.35 (d, J=9.3 Hz, 1H), 7.08-6.87 (m, 2H), 6.46 (s, 1H), 6.09 (s, 1H), 5.61 (s, 2H), 5.48 (s, 1H), 4.42 (d, J=5.1 Hz, 2H), 4.21 (d, J=3.3 Hz, 2H), 2.25 (s, 3H), 2.12 (s, 3H), 1.93-1.83 (m, 1H), 0.91-0.88 (m, 2H), 0.67-0.63 (m, 2H).

### Example 112

### Synthesis of 2-(((6-(((6-amino-2,4-dimethylpyridin-3-yl)methyl)amino)pyrimidin-4-yl)oxy)methyl)-6-cyclopropylimidazo[1,2-a]pyridine-8-carbonitrile (I-112)

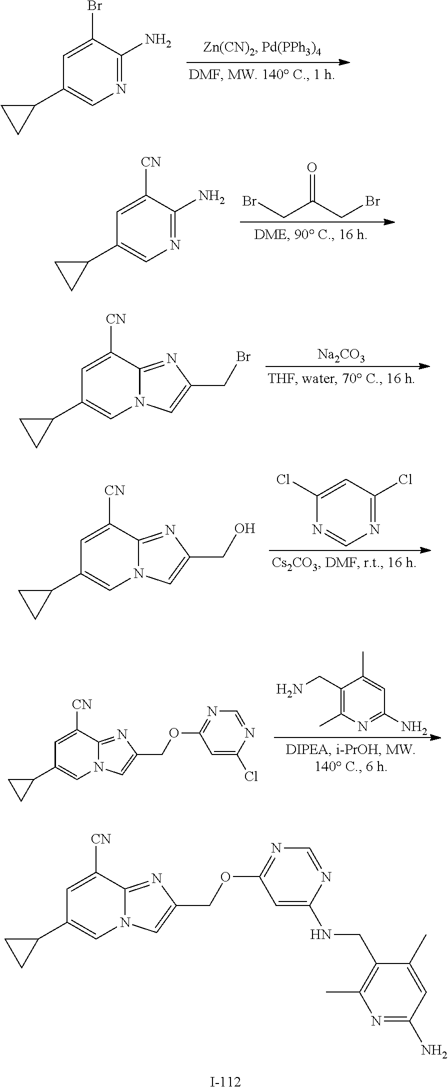

Synthesis of 2-amino-5-cyclopropylnicotinonitrile. A mixture of 3-bromo-5-cyclopropylpyridin-2-amine (636 mg, 3.0 mmol), Zn(CN)₂ (522 mg, 4.5 mmol) and Pd(PPh₃)₄ (173 mg, 0.15 mmol) in DMF (5 mL) was stirred in a sealed tube. After degassing with N₂ for 1 min, the reaction was irradiated in microwave at 140° C. for 1 h. The mixture was concentrated to remove DMF and diluted with DCM/MeOH (10/1, 30 mL). The mixture was filtered through Celite® and the filter cake was washed with DCM/MeOH (10/1, 30 mL). The filtrate was concentrated to give the crude, which was purified by column chromatography (eluent: DCM/MeOH=30/1) to give 2-amino-5-cyclopropylnicotinonitrile (320 mg, yield: 67%) as a yellow solid. ESI-MS [M+H]⁺: 160.1.

Synthesis of 2-(bromomethyl)-6-cyclopropylimidazo[1,2-a]pyridine-8-carbonitrile. A mixture of 2-amino-5-cyclopropylnicotinonitrile (318 mg, 2.0 mmol) and 1,3-dibromopropan-2-one (1.3 g, 6.0 mmol) in DME (20 mL) was stirred at 90° C. for 16 h under N₂. The mixture was concentrated to give the crude, which was purified by column chromatography (eluent: DCM/MeOH=50/1) to give 2-(bromomethyl)-6-cyclopropylimidazo[1,2-a]pyridine-8-carbonitrile (400 mg, yield: 73%) as a yellow solid. ESI-MS [M+H]⁺: 276.2.

Synthesis of 6-cyclopropyl-2-(hydroxymethyl)imidazo[1,2-a]pyridine-8-carbonitrile. A mixture of 2-(bromomethyl)-6-cyclopropylimidazo[1,2-a]pyridine-8-carbonitrile (400 mg, 1.45 mmol) and Na₂CO₃ (461 mg, 4.35 mmol) in THF/water (10 mL/10 mL) was stirred at 70° C. for 16 h. The reaction mixture was cooled to room temperature. H₂O (50 mL) was added and the mixture was extracted with EtOAc/MeOH (10/1, 30 mL×3). The combined organic layers were washed with brine (50 mL), dried over anhydrous Na₂SO₄, and concentrated in vacuo to give the crude, which was purified by Prep-TLC (eluent: DCM/MeOH=20/1) to give 6-cyclopropyl-2-(hydroxymethyl)imidazo[1,2-a]pyridine-8-carbonitrile (170 mg, yield: 55%) as a yellow solid. ESI-MS [M+H]⁺: 214.2.

Synthesis of 2-(((6-chloropyrimidin-4-yl)oxy)methyl)-6-cyclopropylimidazo[1,2-a]pyridine-8-carbonitrile. A mixture of 6-cyclopropyl-2-(hydroxymethyl)imidazo[1,2-a]pyridine-8-carbonitrile (64 mg, 0.3 mmol), 4,6-dichloropyrimidine (67 mg, 0.45 mmol) and Cs₂CO₃ (196 mg, 0.6 mmol) in DMF (2.5 mL) was stirred at room temperature for 16 h. H₂O (50 mL) was added and the mixture was extracted with EtOAc (30 mL×3). The combined organic layers were washed with brine (50 mL), dried over anhydrous Na₂SO₄, and concentrated in vacuo to give the crude, which was purified by Prep-TLC (eluent: DCM/MeOH=20/1) to give 2-(((6-chloropyrimidin-4-yl)oxy)methyl)-6-cyclopropylimidazo[1,2-a]pyridine-8-carbonitrile (30 mg, yield: 31%) as a yellow solid. ESI-MS [M+H]⁺: 326.1.

Synthesis of 2-(((6-(((6-amino-2,4-dimethylpyridin-3-yl)methyl)amino)pyrimidin-4-yl)oxy)methyl)-6-cyclopropylimidazo[1,2-a]pyridine-8-carbonitrile. A mixture of 2-(((6-chloropyrimidin-4-yl)oxy)methyl)-6-cyclopropylimidazo[1,2-a]pyridine-8-carbonitrile (33 mg, 0.1 mmol), 5-(aminomethyl)-4,6-dimethylpyridin-2-amine (23 mg, 0.15 mmol) and DIPEA (65 mg, 0.5 mmol) in i-PrOH (2 mL) was stirred in a sealed tube. After degassing with N₂ for 1 min, the reaction was irradiated in microwave at 140° C. for 6 h. The mixture was concentrated to give the crude, which was purified by Prep-HPLC to give 2-(((6-(((6-amino-2,4-dimethylpyridin-3-yl)methyl)amino)pyrimidin-4-yl)oxy)methyl)-6-cyclopropylimidazo[1,2-a]pyridine-8-carbonitrile as formate salt (10 mg, yield: 21%) as a white solid. ESI-MS [M+H]+: 441.1. ¹H NMR (400 MHz, DMSO) δ8.68 (s, 1H), 8.26-8.22 (m, 2H), 7.98 (s, 1H), 7.78 (s, 1H), 7.06 (s, 1H), 6.12 (s, 1H), 5.85 (s, 1H), 5.67 (s, 2H), 5.41 (s, 2H), 4.29 (s, 2H), 2.27 (s, 3H), 2.14 (s, 3H), 2.04-1.92 (m, 1H), 1.08-0.89 (m, 2H), 0.78-0.74 (m, 2H).

### Example 113

### Synthesis of (2-(((6-(((6-amino-2,4-dimethylpyridin-3-yl)methyl)amino)pyrimidin-4-yl)oxy)methyl)-6-cyclopropylimidazo[1,2-a]pyridin-8-yl)methanol (I-113)

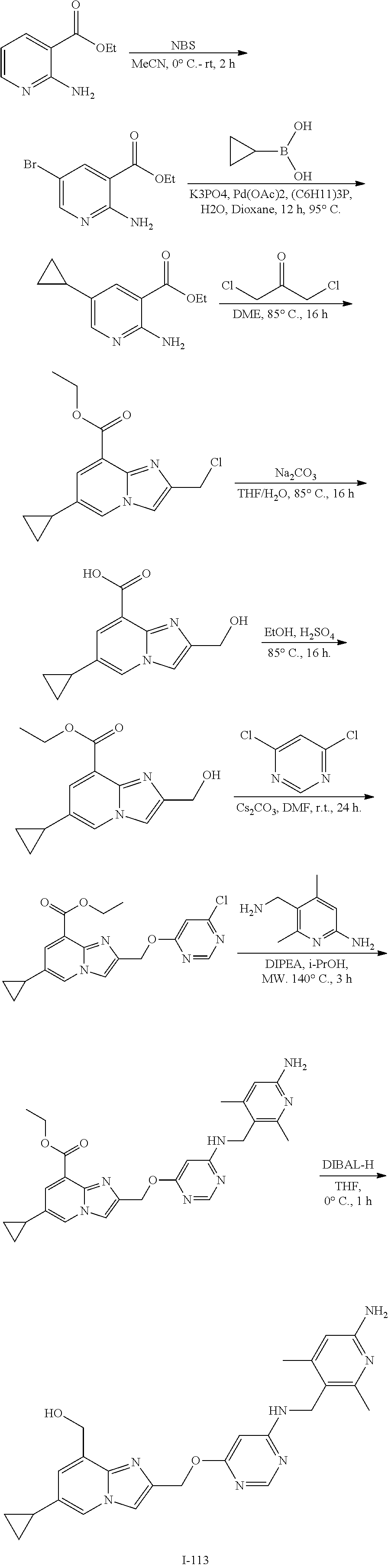

Synthesis of ethyl 2-amino-5-bromonicotinate. To a solution of ethyl 2-ammonicotinate (2.0 g, 12.0 mmol) in MeCN (20 mL) was added a solution of NBS (2.56 g, 14.4 mmol) in MeCN (10 mL) at 0° C. The resulting mixture was stirred at room temperature for 2 h. Water (50 mL) was added and the mixture was extracted with EtOAc (50 mL×3). The combined organic layers were washed with brine, dried over Na₂SO₄ and concentrated to give the crude product, which was purified by column chromatography (eluent: EA/PE=1/2) to give ethyl 2-amino-5-bromonicotinate (2.5 g, yield: 85%) as a yellow solid. ESI-MS [M+H]+: 245.1.

Synthesis of ethyl 2-amino-5-cyclopropylnicotinate. To a solution of ethyl 2-amino-5-bromonicotinate (2.0 g, 8.2 mmol) in dioxane/H₂O (40 mL/4 mL) was added cyclopropylboronic acid (1.41 g, 16.4 mmol), K₃PO₄ (3.48 g, 16.4 mmol), Pd(OAc)₂ (184 mg, 0.82 mmol) and Tricyclohexyl phosphine (230 mg, 0.82 mmol). The resulting mixture was stirred at 95° C. for 12 h under N₂. Water (100 mL) was added and the mixture was extracted with EtOAc (100 mL×3). The combined organic layers were washed with brine, dried over Na₂SO₄ and concentrated to give the crude product, which was purified by column chromatography (eluent: EA/PE=1/2) to give ethyl 2-amino-5-cyclopropylnicotinate (1.3 g, yield: 77%) as a yellow solid. ESI-MS [M+H]⁺: 207.2.

Synthesis of ethyl 2-(chloromethyl)-6-cyclopropylimidazo[1,2-a]pyridine-8-carboxylate. A mixture of ethyl 2-amino-5-cyclopropylnicotinate (1 g, 4.85 mmol) and 1,3-dichloropropan-2-one (1.8 g, 14.55 mmol) in DME (50 mL) was stirred at 85° C. for 16 h under N₂. The mixture was cooled to room temperature and concentrated in vacuo, the residue was purified by column chromatography (eluent: PE/EtOAc=5/1) to give ethyl 2-(chloromethyl)-6-cyclopropylimidazo[1,2-a]pyridine-8-carboxylate (1.0 g, yield: 74%) as a yellow solid. ESI-MS [M+H]⁺: 279.1.

Synthesis of 6-cyclopropyl-2-(hydroxymethyl)imidazo[1,2-a]pyridine-8-carboxylic acid. A mixture of ethyl 2-(chloromethyl)-6-cyclopropylimidazo[1,2-a]pyridine-8-carboxylate (1.0 g, 3.6 mmol) and Na₂CO₃ (763 mg, 7.2 mmol) in THF/H₂O (20 mL/20 mL) was stirred at 85° C. for 16 h. The reaction mixture was cooled to room temperature and quenched with HCl (1M aq., 20 mL) and then extracted with CHCl₃/i-PrOH (3/1, 30 mL×3). The combined organic layers were dried over Na₂SO₄, and concentrated in vacuo to give 6-cyclopropyl-2-(hydroxymethyl)imidazo[1,2-a]pyridine-8-carboxylic acid (750 mg, yield: 90%) as a yellow solid, which was used in the next step without purification. ESI-MS [M+H]⁺: 233.2.

Synthesis of ethyl 6-cyclopropyl-2-(hydroxymethyl)imidazo[1,2-a]pyridine-8-carboxylate. To a solution of 6-cyclopropyl-2-(hydroxymethyl)imidazo[1,2-a]pyridine-8-carboxylic acid (750 mg, 3.2 mmol) in EtOH (30 mL) was added H₂SO₄ (conc., 3 mL) at 0° C. The mixture was stirred at 85° C. for 16 h. The mixture was cooled to room temperature and concentrated in vacuo, the residue was purified by column chromatography (eluent: DCM/MeOH=20/1) to give ethyl 6-cyclopropyl-2-(hydroxymethyl)imidazo[1,2-a]pyridine-8-carboxylate (440 mg, yield: 47% over 2 steps) as a yellow oil. ESI-MS [M+H]+: 261.2.

Synthesis of ethyl 2-(((6-chloropyrimidin-4-yl)oxy)methyl)-6-cyclopropylimidazo[1,2-a]pyridine-8-carboxylate. A mixture of ethyl 6-cyclopropyl-2-(hydroxymethyl)imidazo[1,2-a]pyridine-8-carboxylate (440 mg, 1.7 mmol), 4,6-dichloropyrimidine (503 mg, 3.4 mmol) and Cs₂CO₃ (1.66 g, 5.1 mmol) in DMF (10 mL) was stirred at room temperature for 24 h. H₂O (100 mL) was added and the mixture was extracted with EtOAc (30 mL×3). The combined organic layers were washed with brine (50 mL), dried over anhydrous Na₂SO₄, and concentrated in vacuo, the residue was purified by column chromatography (eluent: DCM/MeOH=30/1) to give ethyl 2-(((6-chloropyrimidin-4-yl)oxy)methyl)-6-cyclopropylimidazo[1,2-a]pyridine-8-carboxylate (360 mg, yield: 57%) as a yellow oil. ESI-MS [M+H]+: 373.2.

Synthesis of ethyl 2-(((6-(((6-amino-2,4-dimethylpyridin-3-yl)methyl)amino)pyrimidin-4-yl)oxy)methyl)-6-cyclopropylimidazo[1,2-a]pyridine-8-carboxylate. A mixture of ethyl 2-(((6-chloropyrimidin-4-yl)oxy)methyl)-6-cyclopropylimidazo[1,2-a]pyridine-8-carboxylate (180 mg, 0.48 mmol), 5-(aminomethyl)-4,6-dimethylpyridin-2-amine (109 mg, 0.72 mmol) and DIPEA (310 mg, 2.4 mmol) in i-PrOH (5 mL) was stirred in a sealed tube. After degassing with N₂ for 1 min, the reaction was irradiated in microwave at 140° C. for 3 h. The mixture was concentrated to give the crude, which was purified by Prep-TLC (eluent: DCM/MeOH=10/1) to give ethyl 2-(((6-(((6-amino-2,4-dimethylpyridin-3-yl)methyl)amino)pyrimidin-4-yl)oxy)methyl)-6-cyclopropylimidazo[1,2-a]pyridine-8-carboxylate (50 mg, yield: 21%) as a yellow solid. ESI-MS [M+H]+: 488.2.

Synthesis of (2-(((6-(((6-amino-2,4-dimethylpyridin-3-yl)methyl)amino)pyrimidin-4-yl)oxy)methyl)-6-cyclopropylimidazo[1,2-a]pyridin-8-yl)methanol. To a solution of 2-(((6-(((6-amino-2,4-dimethylpyridin-3-yl)methyl)amino)pyrimidin-4-yl)oxy)methyl)-6-cyclopropylimidazo (50 mg, 0.10 mmol) in THF (5 mL) was added DIBAL-H (1M solution in THF, 0.3 mL, 0.30 mmol) at 0° C. under N₂ and stirred at 0° C. for 1 h. The reaction was quenched with NH₄Cl (sat. aq., 20 mL) and extracted with EtOAc (30 mL×3). The combined organic layers were washed with brine (30 mL), dried over anhydrous Na₂SO₄, and concentrated in vacuo. The residue was purified by Prep-HPLC to give (2-(((6-(((6-amino-2,4-dimethylpyridin-3-yl)methyl)amino)pyrimidin-4-yl)oxy)methyl)-6-cyclopropylimidazo[1,2-a]pyridin-8-yl)methanol as formate salt (3.4 mg, yield: 7%) as a white solid. ESI-MS [M+H]+: 446.2. ¹H NMR (400 MHz, DMSO) δ8.25-8.21 (m, 2H), 8.19 (s, 2H), 7.81 (s, 1H), 7.05-7.03 (m, 1H), 6.99 (s, 1H), 6.13 (s, 1H), 5.83 (s, 1H), 5.70 (s, 1H), 5.33 (s, 2H), 4.77 (s, 2H), 4.29-4.27 (m, 2H), 2.27 (s, 3H), 2.14 (s, 3H), 1.95-1.93 (m, 1H), 0.95-0.90 (m, 2H), 0.68-0.64 (m, 2H).

### Example 114

### Synthesis of (E)-3-(3-chlorophenyl)-N-(4-(((6-cyclopropylimidazo[1,2-a]pyridin-2-yl)methyl)amino)pyridin-2-yl)acrylamide (I-114)

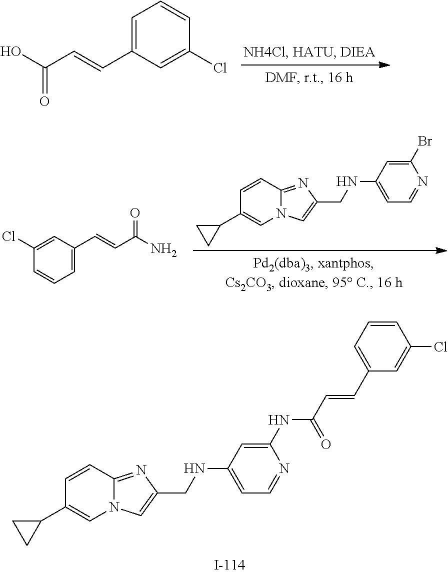 [SMILES: O=C(O)/C=C/c1cccc(Cl)c1 ; NC(=O)/C=C/c1cccc(Cl)c1 ; Brc1cc(NCc2cn3cc(C4CC4)ccc3n2)ccn1 ; O=C(/C=C/c1cccc(Cl)c1)Nc1cc(NCc2cn3cc(C4CC4)ccc3n2)ccn1]

Synthesis of (E)-3-(3-chlorophenyl)acrylamide. A mixture of (E)-3-(3-chlorophenyl)acrylic acid (1.0 g, 5.5 mmol), NH₄Cl (583 mg, 11 mmol), HATU (3.2 g, 8.3 mmol) and DIPEA (2.1 g, 16.5 mmol) in DMF (10 mL) was stirred at room temperature for 16 h. The reaction was quenched with water (100 mL) and extracted with EtOAc (50 mL×3). The combined organic layers were washed with brine (50 mL), dried over Na₂SO₄ and concentrated in vacuo to give the crude, which was purified by column chromatography (EtOAc/PE from 0 to 30%) to (E)-3-(3-chlorophenyl)acrylamide (400 mg, yield: 40%) as a white solid. ESI-MS [M+H]+: 182.0.

Synthesis of (E)-3-(3-chlorophenyl)-N-(4-(((6-cyclopropylimidazo[1,2-a]pyridin-2-yl)methyl)amino)pyridin-2-yl)acrylamide. A mixture of 2-bromo-N-((6-cyclopropylimidazo[1,2-a]pyridin-2-yl)methyl)pyridin-4-amine (50 mg, 0.15 mmol) (synthesis reported in Example 128), (E)-3-(3-chlorophenyl)acrylamide (33 mg, 0.18 mmol), Pd₂(dba)₃ (14 mg, 0.015 mmol), XantPhos (17 mg, 0.03 mmol) and Cs₂CO₃ (147 mg, 0.45 mmol) in dioxane (5 mL) was stirred at 95° C. for 16 h under nitrogen. After cooling to room temperature, the reaction mixture was filtered through Celite® and the filter cake was washed with DCM/MeOH (10/1, 50 mL). The filtrate was concentrated to give the crude, which was purified by Prep-TLC (eluent: DCM/MeOH=15/1) to give (E)-3-(3-chlorophenyl)-N-(4-(((6-cyclopropylimidazo[1,2-a]pyridin-2-yl)methyl)amino)pyridin-2-yl)acrylamide (20 mg, yield: 30%) as a white solid. ESI-MS [M+H]+: 444.1. ¹H NMR (400 MHz, DMSO) δ10.21 (s, 1H), 8.31 (s, 1H), 7.80 (d, J=5.7 Hz, 1H), 7.65 (s, 2H), 7.60-7.51 (m, 3H), 7.51-7.43 (m, 2H), 7.38 (d, J=9.3 Hz, 1H), 7.20 (s, 1H), 7.06 (d, J=15.8 Hz, 1H), 6.97 (d, J=9.3 Hz, 1H), 6.35 (d, J=4.1 Hz, 1H), 4.38 (d, J=5.6 Hz, 2H), 1.96-1.85 (m, 1H), 0.97-0.85 (m, 2H), 0.71-0.61 (m, 2H).

### Example 115

### Synthesis of 2-(6-chloro-JH-indol-1-yl)-N-(4-(((6-cyclopropylimidazo[1,2-a]pyridin-2-yl)methyl)amino)pyridin-2-yl)acetamide (I-115)

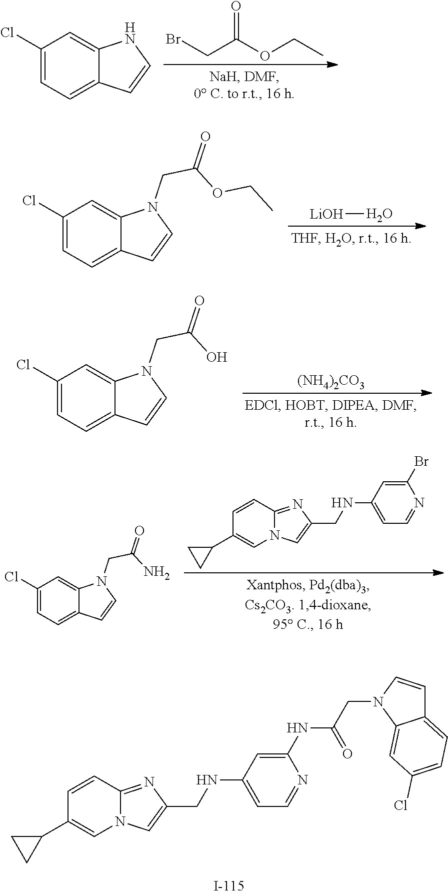

Synthesis of ethyl 2-(6-chloro-1H-indol-1-yl)acetate. To a solution of 6-chloro-1H-indole (500 mg, 3.3 mmol) in DMF (10 mL) was added NaH (200 mg, 5.0 mmol, 60% in mineral oil) at 0° C. and the mixture was stirred at 0° C. for 0.5 h. A solution of ethyl 2-bromoacetate (830 mg, 5.0 mmol) in DMF (5 mL) was added, the resulting mixture was stirred at room temperature for 16 h. H₂O (50 mL) was added and extracted with EtOAc (30 mL×3). The combined organic layers were washed with brine (30 mL), dried over anhydrous Na₂SO₄, and concentrated to give the crude product, which was purified by column chromatography (eluent: PE/EtOAc=5/1) to give ethyl 2-(6-chloro-1H-indol-1-yl)acetate (650 mg, yield: 83%) as a yellow oil. ESI-MS [M+H]+: 238.2.

Synthesis of 2-(6-chloro-1H-indol-1-yl)acetic acid. A mixture of ethyl 2-(6-chloro-1H-indol-1-yl)acetate (650 mg, 2.7 mmol) and LiOH—H₂O (227 mg, 5.4 mmol) in THF/H₂O (10 mL/10 mL) was stirred at room temperature for 16 h. pH of the reaction was adjusted to 6 by HCl (1M aq.), the resulting mixture was extracted with EtOAc (30 mL×3). The combined organic layers were washed with brine (30 mL), dried over anhydrous Na₂SO₄, and concentrated in vacuo, the residue was purified by Prep-HPLC to give 2-(6-chloro-1H-indol-1-yl)acetic acid (400 mg, yield: 70%) as a yellow solid. ESI-MS [M+H]⁺: 210.2.

Synthesis of 2-(6-chloro-1H-indol-1-yl)acetamide. A mixture of 2-(6-chloro-1H-indol-1-yl)acetic acid (150 mg, 0.72 mmol), (NH₄)₂CO₃ (691 mg, 7.2 mmol), EDCI (211 mg, 1.1 mmol), HOBT (149 mg, 1.1 mmol) and DIPEA (464 mg, 3.6 mmol) in DMF (10 mL) was stirred at room temperature for 16 h. H₂O (100 mL) was added and the mixture was extracted with EtOAc (30 mL×3). The combined organic layers were washed with brine (60 mL), dried over anhydrous Na₂SO₄, and concentrated in vacuo to give the crude, which was purified by Prep-TLC (DCM/MeOH=10/1) to give 2-(6-chloro-1H-indol-1-yl)acetamide (90 mg, yield: 60%) as a yellow solid. ESI-MS [M+H]+: 209.2.

Synthesis of 2-(6-chloro-1H-indol-1-yl)-N-(4-(((6-cyclopropylimidazo[1,2-a]pyridin-2-yl)methyl)amino)pyridin-2-yl)acetamide. To a mixture of 2-bromo-N-((6-cyclopropylimidazo[1,2-a]pyridin-2-yl)methyl)pyridin-4-amine (92 mg, 0.27 mmol) (synthesis reported in Example 114), 2-(6-chloro-1H-indol-1-yl)acetamide (56 mg, 0.27 mmol) and Cs₂CO₃ (264 mg, 0.81 mmol) in 1,4-dioxane (10 mL) was added Pd₂(dba)₃ (27 mg, 0.03 mmol) and Xantphos (29 mg, 0.05 mmol). After the mixture was stirred at 95° C. for 16 h under N₂, the reaction mixture was cooled to room temperature. The mixture was filtered through Celite® and the filter cake was washed with DCM/MeOH (10/1, 30 mL). The filtrate was concentrated to give the crude, which was purified by column chromatography (eluent: DCM/MeOH=15/1) to give 2-(6-chloro-1H-indol-1-yl)-N-(4-(((6-cyclopropylimidazo[1,2-a]pyridin-2-yl)methyl)amino)pyridin-2-yl)acetamide (30 mg, yield: 24%) as a white solid. ESI-MS [M+H]+: 471.1. ¹H NMR (400 MHz, DMSO) δ10.36 (s, 1H), 8.27 (s, 1H), 7.80 (d, J=5.8 Hz, 1H), 7.59-7.54 (m, 3H), 7.41 (d, J=3.2 Hz, 1H), 7.37-7.31 (m, 2H), 7.11 (t, J=5.8 Hz, 1H), 7.05-7.02 (m, 1H), 6.97-6.92 (m, 1H), 6.48 (d, J=2.8 Hz, 1H), 6.38-6.33 (m, 1H), 5.07 (s, 2H), 4.31 (d, J=5.7 Hz, 2H), 1.95-1.84 (m, 1H), 0.95-0.83 (m, 2H), 0.71-0.59 (m, 2H).

### Example 116

### Synthesis of 2-(5-chloro-2-(1H-tetrazol-1-yl)phenoxy)-N-(4-(((6-cyclopropylimidazo[1,2-a]pyridin-2-yl)methyl amino)pyridin-2-yl)acetamide (I-116)

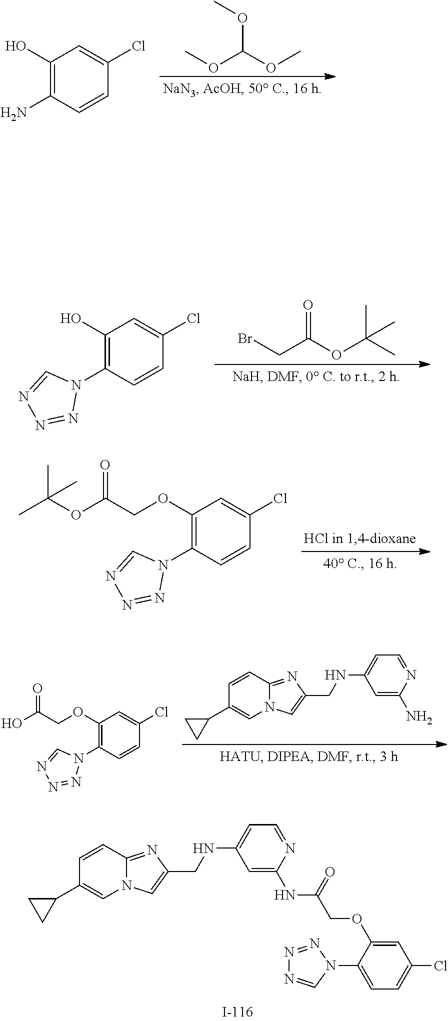

Synthesis of 5-chloro-2-(1H-tetrazol-1-yl)phenol. To a solution of 2-amino-5-chlorophenol (500 mg, 3.5 mmol) and trimethoxymethane (1.1 g, 10.5 mmol) in AcOH (10 mL) was added NaN₃ (683 mg, 10.5 mmol) at room temperature. The reaction mixture was stirred at 50° C. for 16 h. The mixture was cooled to room temperature, quenched with NaHCO₃ (sat. aq., 50 mL) and extracted with EtOAc (30 mL×3). The combined organic layers were washed with brine (30 mL), dried over anhydrous Na₂SO₄, and concentrated to give the crude product, which was purified by column chromatography (eluent: DCM/MeOH=20/1) to give 5-chloro-2-(1H-tetrazol-1-yl)phenol (200 mg, yield: 29%) as a brown solid. ESI-MS [M+H]+: 197.1.

Synthesis of tert-butyl 2-(5-chloro-2-(1H-tetrazol-1-yl)phenoxy)acetate. To a solution of 5-chloro-2-(1H-tetrazol-1-yl)phenol (200 mg, 1.0 mmol) in DMF (5 mL) was added NaH (48 mg, 1.2 mmol, 60% in mineral oil) at 0° C. After the reaction mixture was stirred at 0° C. for 30 min, a solution of tert-butyl 2-bromoacetate (233 mg, 1.2 mmol) in DMF (2 mL) was added. The mixture was stirred at room temperature for 2 h. Water (50 mL) was added and the mixture was extracted with EtOAc (20 mL×3). The combined organic layers were washed with brine (30 mL), dried over anhydrous Na₂SO₄, and concentrated to give the crude product, which was purified by Prep-TLC (eluent: DCM/MeOH=30/1) to give tert-butyl 2-(5-chloro-2-(1H-tetrazol-1-yl)phenoxy)acetate (260 mg, yield: 84%) as a yellow solid. ESI-MS [M+H]+: 311.2.

Synthesis of 2-(5-chloro-2-(1H-tetrazol-1-yl)phenoxy)acetic acid. A mixture of tert-butyl 2-(5-chloro-2-(1H-tetrazol-1-yl)phenoxy)acetate (100 mg, 0.32 mmol) in HCl (4M solution in 1,4-dioxane, 5 mL) was stirred at 40° C. for 16 h. The reaction mixture was concentrated to give 2-(5-chloro-2-(1H-tetrazol-1-yl)phenoxy)acetic acid (70 mg, yield: 86%) as a yellow solid which was used in the next step without purification. ESI-MS [M+H]⁺: 255.2.

Synthesis of 2-(5-chloro-2-(1H-tetrazol-1-yl)phenoxy)-N-(4-(((6-cyclopropylimidazo[1,2-a]pyridin-2-yl)methyl)amino)pyridin-2-yl)acetamide. A mixture of 2-(5-chloro-2-(1H-tetrazol-1-yl)phenoxy)acetic acid (70 mg, 0.28 mmol), N⁴-((6-cyclopropylimidazo[1,2-a]pyridin-2-yl)methyl)pyridine-2,4-diamine (78 mg, 0.28 mmol), HATU (160 mg, 0.42 mmol) and DIPEA (108 mg, 0.84 mmol) in DMF (3 mL) was stirred at room temperature for 3 h. The reaction mixture was quenched with water (30 mL) and extracted with EtOAc (30 mL×3). The combined organic layers were washed with brine (30 mL), dried over anhydrous Na₂SO₄, and concentrated in vacuo, the residue was purified by Prep-TLC (eluent: DCM/MeOH=10/1) to give 2-(5-chloro-2-(1H-tetrazol-1-yl)phenoxy)-N-(4-(((6-cyclopropylimidazo[1,2-a]pyridin-2-yl)methyl)amino)pyridin-2-yl)acetamide (10.4 mg, yield: 7.2%) as a yellow solid. ESI-MS [M+H]⁺: 516.2. ¹H NMR (400 MHz, DMSO) δ10.22 (s, 1H), 9.96 (s, 1H), 8.30 (s, 1H), 7.81 (dd, J=14.4, 7.2 Hz, 2H), 7.63 (s, 1H), 7.45 (d, J=1.8 Hz, 1H), 7.38-7.30 (m, 3H), 7.23-7.16 (m, 1H), 6.96 (d, J=9.3 Hz, 1H), 6.36 (d, J=4.2 Hz, 1H), 5.01 (s, 2H), 4.35 (d, J=5.5 Hz, 2H), 1.93-1.86 (m, 1H), 0.93-0.88 (m, 2H), 0.68-0.64 (m, 2H).

### Example 117

### Synthesis of 2-((5-chloro-2-(1H-tetrazol-1-yl)phenyl)amino)-N-(4-(((6-cyclopropylimidazo[1,2-a]pyridin-2-yl)methyl)amino)pyridin-2-yl)acetamide

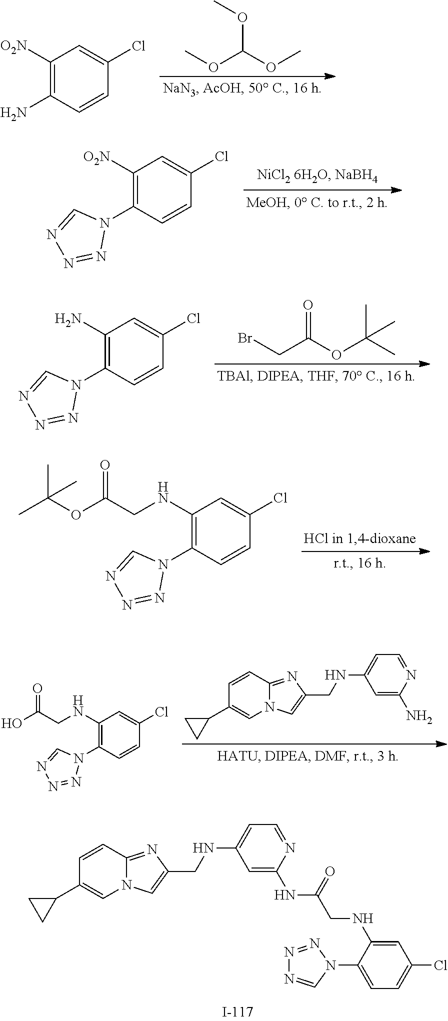

Synthesis of 1-(4-chloro-2-nitrophenyl)-1H-tetrazole. To a solution of 4-chloro-2-nitroaniline (500 mg, 2.9 mmol) and trimethoxymethane (922 mg, 8.7 mmol) in AcOH (10 mL) was added NaN₃ (566 mg, 8.7 mmol) at room temperature. The reaction mixture was stirred at 50° C. for 16 h. The mixture was cooled to room temperature, quenched with NaHCO₃ (sat. aq., 50 mL) and extracted with EtOAc (30 mL×3). The combined organic layers were washed with brine (30 mL), dried over anhydrous Na₂SO₄, and concentrated to give the crude product, which was purified by column chromatography (eluent: DCM/MeOH=30/1) to give 1-(4-chloro-2-nitrophenyl)-1H-tetrazole (350 mg, yield: 54%) as a yellow solid. ESI-MS [M+H]+: 226.1.

Synthesis of 5-chloro-2-(1H-tetrazol-1-yl)aniline. To a solution of 1-(4-chloro-2-nitrophenyl)-1H-tetrazole (350 mg, 1.56 mmol) and NiCl₂ 6H₂O (38 mg, 0.16 mmol) in MeOH (10 mL) was added NaBH₄ (178 mg, 4.68 mmol) at 0° C. The reaction mixture was stirred at room temperature for 2 h. Water (50 mL) was added and the reaction was extracted with EtOAc (30 mL×3). The combined organic layers were washed with brine (30 mL), dried over anhydrous Na₂SO₄, and concentrated to give the crude, which was purified by column chromatography (eluent: DCM/MeOH=10/1) to give 5-chloro-2-(1H-tetrazol-1-yl)aniline (200 mg, yield: 66%) as a yellow solid. ESI-MS [M+H]+: 196.2.

Synthesis of tert-butyl (5-chloro-2-(1H-tetrazol-1-yl)phenyl)glycinate. To a solution of 5-chloro-2-(1H-tetrazol-1-yl)aniline (200 mg, 1.0 mmol), TBAI (74 mg, 0.2 mmol) and DIPEA (387 mg, 3.0 mmol) in THF (5 mL) was added tert-butyl 2-bromoacetate (213 mg, 1.1 mmol) and the mixture was stirred at 70° C. for 16 h. The reaction mixture was cooled to room temperature. Water (50 mL) was added and extracted with EtOAc (30 mL×3). The combined organic layers were washed with brine (30 mL), dried over anhydrous Na₂SO₄, and concentrated to give the crude product, which was purified by Prep-TLC (eluent: DCM/MeOH=30/1) to give tert-butyl (5-chloro-2-(1H-tetrazol-1-yl)phenyl)glycinate (160 mg, yield: 52%) as a yellow solid. ESI-MS [M+H]+: 310.2.

Synthesis of (5-chloro-2-(1H-tetrazol-1-yl)phenyl)glycine. A mixture of tert-butyl (5-chloro-2-(1H-tetrazol-1-yl)phenyl)glycinate (160 mg, 0.5 mmol) in HCl (4M solution in 1,4-dioxane, 5 mL) was stirred at room temperature for 16 h. The reaction mixture was concentrated to give (5-chloro-2-(1H-tetrazol-1-yl)phenyl)glycine (100 mg, yield: 79%) as a yellow solid, which was used in the next step without purification. ESI-MS [M+H]⁺: 254.2.

Synthesis of 2-((5-chloro-2-(1H-tetrazol-1-yl)phenyl)amino)-N-(4-(((6-cyclopropylimidazo[1,2-a]pyridin-2-yl)methyl)amino)pyridin-2-yl)acetamide. A mixture of (5-chloro-2-(1H-tetrazol-1-yl)phenyl)glycine (100 mg, 0.4 mmol), N⁴-((6-cyclopropylimidazo[1,2-a]pyridin-2-yl)methyl)pyridine-2,4-diamine (112 mg, 0.4 mmol), HATU (228 mg, 0.6 mmol) and DIPEA (155 mg, 1.2 mmol) in DMF (5 mL) was stirred at room temperature for 3 h. The reaction mixture was quenched with water (50 mL) and extracted with EtOAc (30 mL×3). The combined organic layers were washed with brine (30 mL), dried over anhydrous Na₂SO₄, and concentrated in vacuo to give the crude, which was purified by Prep-TLC (eluent: DCM/MeOH=10/1) to give 2-((5-chloro-2-(1H-tetrazol-1-yl)phenyl)amino)-N-(4-(((6-cyclopropylimidazo[1,2-a]pyridin-2-yl)methyl)amino)pyridin-2-yl)acetamide (8.7 mg, yield: 4.2%) as a white solid. ESI-MS [M+H]+: 515.1. ¹H NMR (400 MHz, DMSO) δ9.99 (s, 1H), 9.78 (s, 1H), 8.29 (d, J=0.8 Hz, 1H), 7.76 (d, J=5.8 Hz, 1H), 7.62 (s, 1H), 7.38-7.34 (m, 3H), 7.14 (t, J=5.8 Hz, 1H), 6.96 (dd, J=9.3, 1.8 Hz, 1H), 6.87-6.75 (m, 2H), 6.33 (dd, J=5.8, 2.1 Hz, 1H), 6.09 (t, J=6.0 Hz, 1H), 4.34 (d, J=5.7 Hz, 2H), 3.96 (d, J=6.1 Hz, 2H), 1.92-1.87 (m, 1H), 0.91-0.89 (m, 2H), 0.72-0.62 (m, 2H).

### Example 118

### Synthesis of 7-chloro-N-(4-(((6-cyclopropylimidazo[1,2-a]pyridin-2-yl)methyl)amino)pyridin-2-yl)-2-naphthamide (I-118)

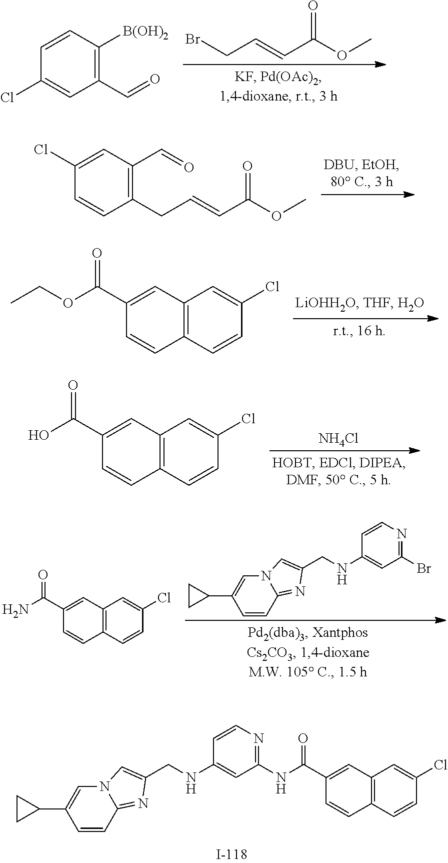

Synthesis of methyl (E)-4-(4-chloro-2-formylphenyl)but-2-enoate. A mixture of (4-chloro-2-formylphenyl)boronic acid (1.84 g, 10.0 mmol) and KF (1.74 g, 30.0 mmol) in 1,4-dioxane (50 mL) was stirred at room temperature for 30 min under N₂. Then a mixture of methyl (E)-4-bromobut-2-enoate (1.78 g, 10.0 mmol) and Pd(OAc)₂ (225 mg, 1.0 mmol) in 1,4-dioxane (10 mL) was added. The resulting mixture was stirred at room temperature for 16 h under N₂. Water (100 mL) was added and the reaction was extracted with EtOAc (50 mL×3). The combined organic layers were washed with brine (50 mL), dried over anhydrous Na₂SO₄, and concentrated in vacuo to give the crude, which was purified by column chromatography (eluent: PE/EtOAc=3/1) to give methyl (E)-4-(4-chloro-2-formylphenyl)but-2-enoate (430 mg, yield: 18%) as a colorless oil. ESI-MS [M+H]+: 239.2.

Synthesis of ethyl 7-chloro-2-naphthoate. A mixture of methyl (E)-4-(4-chloro-2-formylphenyl)but-2-enoate (430 mg, 1.8 mmol) and DBU (410 mg, 2.7 mmol) in EtOH (20 mL) was stirred at 80° C. for 16 under N₂. The reaction mixture was cooled to room temperature and concentrated to give the crude, which was purified by Prep-TLC (eluent: PE/EtOAc=5/1) to give ethyl 7-chloro-2-naphthoate (250 mg, yield: 59%) as a white solid. ESI-MS [M+H]+: 235.1.

Synthesis of 7-chloro-2-naphthoic acid. A mixture of ethyl 7-chloro-2-naphthoate (250 mg, 1.07 mmol) and LiOH—H₂O (134 mg, 3.2 mmol) in THF (10 mL) and H₂O (2 mL) was stirred at room temperature for 16 h. The reaction mixture quenched with HCl (2M aq., 2 mL) and concentrated to give the crude, which was purified by Prep-TLC (eluent: DCM/MeOH=10/1) to give 7-chloro-2-naphthoic acid (206 mg, yield: 94%) as a white solid. ESI-MS [M+H]+: 207.1.

Synthesis of 7-chloro-2-naphthamide. A mixture of 7-chloro-2-naphthoic acid (206 mg, 1.0 mmol), NH₄Cl (265 mg, 5.0 mmol), HOBT (338 mg, 2.5 mmol), EDCI (480 mg, 2.5 mmol) and DIPEA (645 mg, 5.0 mmol) in DMF (10 mL) was stirred at 50° C. for 5 h under N₂. The reaction mixture was cooled to room temperature. Water (50 mL) was added and extracted with EtOAc (30 mL×3). The combined organic layers were washed with brine (50 mL), dried over anhydrous Na₂SO₄, and concentrated in vacuo to give the crude, which was purified by column chromatography (eluent: DCM/MeOH=10/1) to give 7-chloro-2-naphthamide (180 mg, yield: 88%) as a white solid. ESI-MS [M+H]+: 206.2.

Synthesis of 7-chloro-N-(4-(((6-cyclopropylimidazo[1,2-a]pyridin-2-yl)methyl)amino)pyridin-2-yl)-2-naphthamide. A mixture of 7-chloro-2-naphthamide (41 mg, 0.20 mmol), 2-bromo-N-((6-cyclopropylimidazo[1,2-a]pyridin-2-yl)methyl)pyridin-4-amine (103 mg, 0.30 mmol) (synthesis reported in Example 128), Pd₂(dba)₃ (18 mg, 0.02 mmol), Xantphos (23 mg, 0.04 mmol) and Cs₂CO₃ (196 mg, 0.60 mmol) in 1,4-dioxane (5 mL) was stirred in a sealed tube. After degassing with N₂ for 1 min, the reaction was irradiated in microwave at 105° C. for 1.5 h. The reaction mixture was cooled to room temperature. The mixture was filtered through Celite® and the filter cake was washed with DCM/MeOH (10/1, 30 mL). The filtrate was concentrated to give the crude, which was purified by Prep-TLC (eluent: DCM/MeOH=10/1) to give 7-chloro-N-(4-(((6-cyclopropylimidazo[1,2-a]pyridin-2-yl)methyl)amino)pyridin-2-yl)-2-naphthamide (45 mg, yield: 48%) as a white solid. ESI-MS [M+H]⁺: 468.2. ¹H NMR (400 MHz, DMSO) δ10.43 (s, 1H), 8.60 (s, 1H), 8.32 (s, 1H), 8.15 (d, J=2.1 Hz, 1H), 8.05 (t, J=4.4 Hz, 3H), 7.86 (d, J=5.8 Hz, 1H), 7.71-7.57 (m, 3H), 7.39 (d, J=9.3 Hz, 1H), 7.24 (s, 1H), 6.97 (dd, J=9.3, 1.8 Hz, 1H), 6.42 (dd, J=5.8, 2.1 Hz, 1H), 4.42 (d, J=5.7 Hz, 2H), 1.97-1.83 (m, 1H), 0.98-0.84 (m, 2H), 0.76-0.57 (m, 2H).

### Example 119

### Synthesis of 2-(5-chloro-2-oxopyridin-1 (2H)-yl)-N-(6-(((6-cyclopropylimidazo[1,2-a]pyridin-2-yl)methyl)amino)pyrimidin-4-yl)acetamide (I-119)

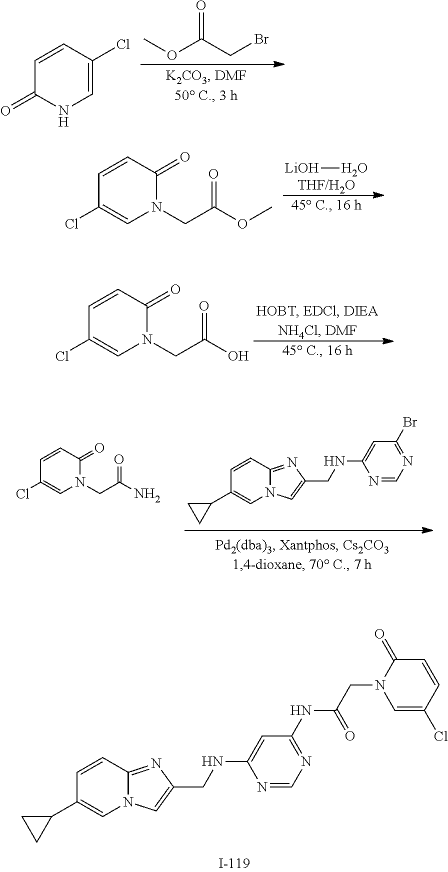

Synthesis of methyl 2-(5-chloro-2-oxopyridin-1 (2H)-yl)acetate. A mixture of 5-chloropyridin-2 (1H)-one (645 mg, 5.0 mmol), methyl 2-bromoacetate (912 mg, 6.0 mmol) and K₂CO₃ (2.1 g, 15.0 mmol) in DMF (20 mL) was stirred at 50° C. for 3 h. The reaction mixture was cooled to room temperature. Water (50 mL) was added and extracted with EtOAc (30 mL×3). The combined organic layers were washed with brine (50 mL), dried over anhydrous Na₂SO₄, and concentrated to give the crude product, which was purified by column chromatography (eluent: PE/EtOAc=4/1) to give methyl 2-(5-chloro-2-oxopyridin-1 (2H)-yl)acetate (450 mg, yield: 45%) as a yellow oil. ESI-MS: [M+H]⁺, 202.1.

Synthesis of 2-(5-chloro-2-oxopyridin-1 (2H)-yl)acetic acid. A mixture of methyl 2-(5-chloro-2-oxopyridin-1 (2H)-yl)acetate (400 mg, 2.0 mmol) and LiOH—H₂O (252 mg, 6.0 mmol) in THF (5 mL) and H₂O (1 mL) was stirred at 45° C. for 16 h. The reaction mixture was cooled to room temperature and was quenched with HCl (1M aq., 10 mL) and concentrated to give the crude, which was purified by Prep-TLC (eluent: DCM/MeOH=10/1) to give 2-(5-chloro-2-oxopyridin-1 (2H)-yl)acetic acid (350 mg, yield: 94%) as a yellow oil. ESI-MS: [M+H]⁺, 188.1.

Synthesis of 2-(5-chloro-2-oxopyridin-1 (2H)-yl)acetamide. A mixture of 2-(5-chloro-2-oxopyridin-1 (2H)-yl)acetic acid (350 mg, 1.87 mmol), NH₄Cl (198 mg, 3.74 mmol), HOBT (505 mg, 3.74 mmol), EDCI (718 mg, 3.74 mmol) and DIPEA (1.2 g, 9.35 mmol) in DMF (10 ml) was stirred at 45° C. for 16 h. The reaction mixture was cooled to room temperature. Water (50 mL) was added and the rection was extracted with EtOAc (30 mL×3). The combined organic layers were washed with brine (50 mL), dried over anhydrous Na₂SO₄, and concentrated in vacuo to give the crude, which was purified by column chromatography (eluent: DCM/MeOH=15/1) to give 2-(5-chloro-2-oxopyridin-1 (2H)-yl)acetamide (150 mg, yield: 43%) as a white solid. ESI-MS: [M+H]⁺, 187.2.

Synthesis of 2-(5-chloro-2-oxopyridin-1 (2H)-yl)-N-(6-(((6-cyclopropylimidazo[1,2-a]pyridin-2-yl)methyl)amino)pyrimidin-4-yl)acetamide. A mixture of 2-(5-chloro-2-oxopyridin-1 (2H)-yl)acetamide (56 mg, 0.3 mmol), 6-bromo-N-((6-cyclopropylimidazo[1,2-a]pyridin-2-yl)methyl)pyrimidin-4-amine (103 mg, 0.3 mmol) (synthesis reported in Example 99), Pd₂(dba)₃ (27 mg, 0.03 mmol), Xantphos (35 mg, 0.06 mmol) and Cs₂CO₃ (293 mg, 0.9 mmol) in 1,4-dioxane (5 mL) was stirred at 70° C. for 7 h under N₂. The reaction mixture was cooled to room temperature and filtered through Celite®, the filter cake was washed with DCM/MeOH (10/1, 20 mL). The filtrate was concentrated to give the crude, which was purified by Prep-TLC (eluent: DCM/MeOH=15/1) to give 2-(5-chloro-2-oxopyridin-1 (2H)-yl)-N-(6-(((6-cyclopropylimidazo[1,2-a]pyridin-2-yl)methyl)amino)pyrimidin-4-yl)acetamide (20 mg, yield: 15%) as a white solid. ESI-MS: [M+H]⁺, 450.1. ¹H NMR (400 MHz, DMSO) δ10.73 (s, 1H), 8.29 (s, 1H), 8.23 (s, 1H), 8.01-7.84 (m, 2H), 7.60 (s, 1H), 7.55-7.52 (m, 1H), 7.36 (d, J=9.3 Hz, 1H), 7.16 (s, 1H), 6.96-6.94 (m, 1H), 6.45 (d, J=9.7 Hz, 1H), 4.75 (s, 2H), 4.58 (s, 2H), 1.93-1.86 (m, 1H), 0.92-0.88 (m, 2H), 0.67-0.63 (m, 2H).

### Example 120

### Synthesis of 7-chloro-N-(4-(((6-cyclopropylimidazo[1,2-a]pyridin-2-yl)methyl)amino)pyridin-2-yl)-1,2,3,4-tetrahydronaphthalene-2-carboxamide (I-120)

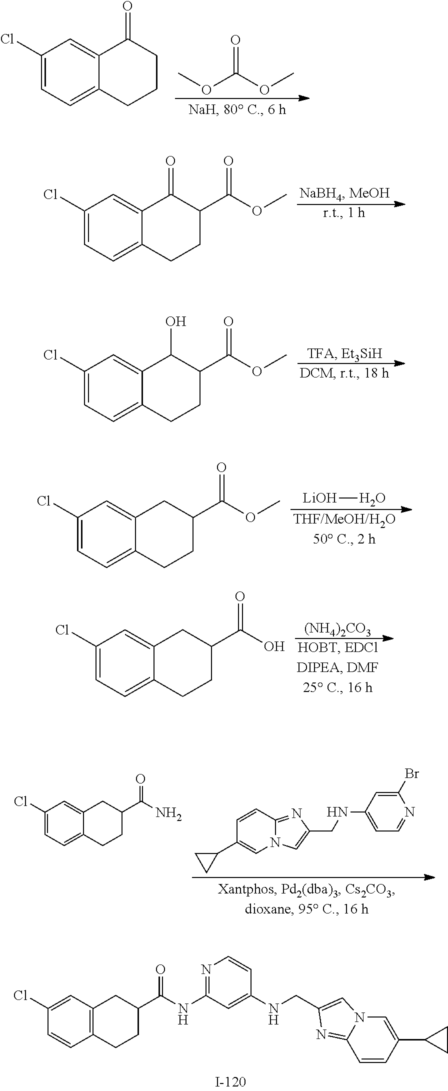

Synthesis of methyl 7-chloro-1-oxo-1,2,3,4-tetrahydronaphthalene-2-carboxylate. To a mixture of 7-chloro-3,4-dihydronaphthalen-1 (2H)-one (2.0 g, 11 mmol) and dimethyl carbonate (2.0 g, 22 mmol) in THF (30 mL) was added NaH (880 mg, 22 mmol, 60% in mineral oil) and the mixture was stirred was stirred at 80° C. and for 6 h. After cooling to room temperature, the reaction was quenched with NH₄Cl (sat. aq., 100 mL) and extracted with EtOAc (50 mL×3). The combined organic layers were washed with brine (50 mL), dried over Na₂SO₄ and concentrated in vacuo to give the crude, which was purified by column chromatography (EtOAc/PE from 0 to 10%) to give methyl 7-chloro-1-oxo-1,2,3,4-tetrahydronaphthalene-2-carboxylate (2.0 g, yield: 76%) as a colorless oil. ESI-MS [M+H]+: 239.0.

Synthesis of methyl 7-chloro-1-hydroxy-1,2,3,4-tetrahydronaphthalene-2-carboxylate. To solution of methyl 7-chloro-1-oxo-1,2,3,4-tetrahydronaphthalene-2-carboxylate (2.0 g, 8.4 mmol) in MeOH (20 mL) was added NaBH₄ (638 mg, 16.8 mmol) and the mixture was stirred at room temperature for 1 h. The reaction was quenched with NH₄Cl (sat. aq., 50 mL) and extracted with EtOAc (50 mL×3). The combined organic layers were washed with brine (50 mL), dried over Na₂SO₄, concentrated to give the crude, which was purified by column chromatography (eluent: EtOAc/PE=0-30%) to give methyl 7-chloro-1-hydroxy-1,2,3,4-tetrahydronaphthalene-2-carboxylate (600 mg, yield: 30%) as a white solid. ESI-MS [M+H]⁺: 241.1.

Synthesis of methyl 7-chloro-1,2,3,4-tetrahydronaphthalene-2-carboxylate. A solution of methyl 7-chloro-1-hydroxy-1,2,3,4-tetrahydronaphthalene-2-carboxylate (600 mg, 2.5 mmol) in DCM/TFA (10 mL/5 mL) was added Et₃SiH (0.5 mL) and stirred at room temperature for 18 h. The reaction was quenched with water (50 mL) and extracted with EtOAc (50 mL×3). The combined organic layers were washed with brine (50 mL), dried over Na₂SO₄, concentrated to give the crude, which was purified by column chromatography (eluent: EtOAc/PE=0-15%) to give methyl 7-chloro-1,2,3,4-tetrahydronaphthalene-2-carboxylate (500 mg, yield: 89%) as a colorless oil. ESI-MS [M+H]⁺: 225.1.

Synthesis of 7-chloro-1,2,3,4-tetrahydronaphthalene-2-carboxylic acid. A mixture of methyl 7-chloro-1,2,3,4-tetrahydronaphthalene-2-carboxylate (500 mg, 2.2 mmol) and LiOH—H₂O (185 mg, 4.4 mmol) in THF/MeOH/H₂O (5 mL/5 mL/5 mL) was stirred at 50° C. for 2 h. After cooling to room temperature, pH of the reaction mixture was adjusted 4-5 with 1M HCl and extracted with EtOAc (50 mL×3). The combined organic layers were washed with brine (50 mL), dried over Na₂SO₄ and concentrated in vacuo to give 7-chloro-1,2,3,4-tetrahydronaphthalene-2-carboxylic acid (200 mg, yield: 43%) as a white solid. ESI-MS [M+H]+: 211.0.

Synthesis of 7-chloro-1,2,3,4-tetrahydronaphthalene-2-carboxamide. A mixture of 7-chloro-1,2,3,4-tetrahydronaphthalene-2-carboxylic acid (200 mg, 0.95 mmol), (NH₄)₂CO₃ (272 mg, 2.85 mmol), HOBT (257 mg, 1.90 mmol), EDCI (361 mg, 1.90 mmol) and DIPEA (490 mg, 3.80 mmol) in DMF (10 mL) was stirred at room temperature for 16 h. The reaction was quenched with water (100 mL) and extracted with EtOAc (100 mL×3). The combined organic layers were washed with brine (100 mL), dried over Na₂SO₄ and concentrated in vacuo to give the crude, which was purified by column chromatography (EtOAc/PE from 0 to 50%) to give 7-chloro-1,2,3,4-tetrahydronaphthalene-2-carboxamide (80 mg, yield: 40%) as a white solid. ESI-MS [M+H]+: 210.1.

Synthesis of 7-chloro-N-(4-(((6-cyclopropylimidazo[1,2-a]pyridin-2-yl)methyl)amino)pyridin-2-yl)-1,2,3,4-tetrahydronaphthalene-2-carboxamide. A mixture of 2-bromo-N-((6-cyclopropylimidazo[1,2-a]pyridin-2-yl)methyl)pyridin-4-amine (50 mg, 0.15 mmol) (synthesis reported in Example 128), 7-chloro-1,2,3,4-tetrahydronaphthalene-2-carboxamide (38 mg, 0.18 mmol), Pd₂(dba)₃ (14 mg, 0.015 mmol), XantPhos (17 mg, 0.03 mmol) and Cs₂CO₃ (147 mg, 0.45 mmol) in dioxane (5 mL) was stirred at 95° C. for 16 h under nitrogen. After cooling to room temperature, the reaction mixture was filtered through Celite® and the filter cake was washed with DCM/MeOH (10/1, 50 mL). The filtrate was concentrated to give the crude, which was purified by Prep-TLC (eluent: DCM/MeOH=15/1) to give 7-chloro-N-(4-(((6-cyclopropylimidazo[1,2-a]pyridin-2-yl)methyl)amino)pyridin-2-yl)-1,2,3,4-tetrahydronaphthalene-2-carboxamide (25 mg, yield: 35%) as a white solid. ESI-MS [M+H]⁺: 472.1. ¹H NMR (400 MHz, DMSO) δ10.09 (s, 1H), 8.31 (s, 1H), 8.22 (s, 1H), 7.76 (d, J=5.8 Hz, 1H), 7.63 (s, 1H), 7.46 (s, 1H), 7.38 (d, J=9.3 Hz, 1H), 7.19 (d, J=1.9 Hz, 1H), 7.16-7.07 (M, 2H), 6.99-6.93 (M, 1H), 6.35-6.29 (M, 1H), 4.36 (d, J=5.6 Hz, 2H), 2.91-2.62 (m, 5H), 2.05-1.95 (m, 1H), 1.94-1.84 (m, 1H), 1.77-1.61 (m, 1H), 0.95-0.85 (M, 2H), 0.70-0.60 (m, 2H).

### Example 121

### Synthesis of N-(6-(((6-cyclopropylimidazo[1,2-a]pyridin-2-yl)methyl)amino)pyrimidin-4-yl)-2-(5-methoxy-2-oxopryidin-1 (2H)-yl)acetamide (I-212)

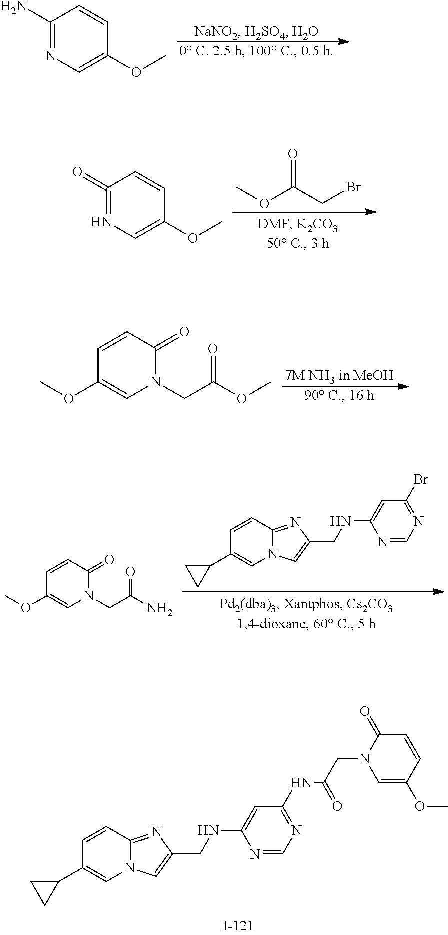

Synthesis of 5-methoxypyridin-2 (1H)-one. To a solution of 5-methoxypyridin-2-amine (3.0 g, 24.2 mmol) in concentrated H₂SO₄ (10 mL) was added a solution of NaNO₂ (2.3 g, 33.9 mmol) in water (40 mL) at 0° C. slowly. The reaction mixture was stirred at 0° C. for 2.5 h, then heated to 100° C. and stirred at 100° C. for 30 min. The reaction mixture was cooled to room temperature and quenched with Na₂CO₃ (sat. aq., 100 mL). The reaction was extracted with EtOAc (100 mL×3). The combined organic layers were washed with brine (50 mL), dried over anhydrous Na₂SO₄, and concentrated to give the crude product, which was purified by column chromatography (eluent: PE/EtOAc=20/1) to give 5-methoxypyridin-2 (1H)-one (1.5 g, yield: 50%) as a yellow solid. ESI-MS: [M+H]⁺, 126.2.

Synthesis of methyl 2-(5-methoxy-2-oxopyridin-1 (2H)-yl)acetate. A mixture of 5-methoxypyridin-2 (1H)-one (700 mg, 5.6 mmol), methyl 2-bromoacetate (942 mg, 6.2 mmol) and K₂CO₃ (2.3 g, 16.8 mmol) in DMF (10 mL) was stirred at 50° C. for 3 h. The reaction mixture was cooled to room temperature. Water (50 mL) was added and extracted with EtOAc (30 mL×3). The combined organic layers were washed with brine (50 mL), dried over anhydrous Na₂SO₄, and concentrated to give the crude product, which was purified by column chromatography (eluent: PE/EtOAc=5/1) to give methyl 2-(5-methoxy-2-oxopyridin-1 (2H)-yl)acetate (200 mg, yield:18%) as a yellow oil. ESI-MS: [M+H]⁺, 198.1.

Synthesis of 2-(5-methoxy-2-oxopyridin-1 (2H)-yl)acetamide. A mixture of methyl 2-(5-methoxy-2-oxopyridin-1 (2H)-yl)acetate (197 mg, 1.0 mmol) in NH₃ (7M in MeOH, 10 mL) was stirred at 90° C. for 16 h. The reaction mixture was cooled to room temperature and concentrated to give the crude, which was purified by column chromatography (eluent: DCM/MeOH=20/1) to give 2-(5-methoxy-2-oxopyridin-1 (2H)-yl)acetamide (160 mg, yield: 88%) as a yellow solid. ESI-MS: [M+H]⁺, 183.1.

Synthesis of N-(6-(((6-cyclopropylimidazo[1,2-a]pyridin-2-yl)methyl)amino)pyrimidin-4-yl)-2-(5-methoxy-2-oxopyridin-1 (2H)-yl)acetamide. A mixture of 2-(5-methoxy-2-oxopyridin-1 (2H)-yl)acetamide (50 mg, 0.27 mmol), 6-bromo-N-((6-cyclopropylimidazo[1,2-a]pyridin-2-yl)methyl)pyrimidin-4-amine (93 mg, 0.27 mmol) (synthesis reported in Example 99), Pd₂(dba)₃ (25 mg, 0.027 mmol), Xantphos (31 mg, 0.054 mmol) and Cs₂CO₃ (264 mg, 0.81 mmol) in 1,4-dioxane (5 mL) was stirred at 60° C. for 5 h under N₂. The reaction mixture was then cooled to room temperature. The mixture was filtered through Celite® and the filter cake was washed with DCM/MeOH (10/1, 30 mL). The filtrate was concentrated to give the crude, which was purified by Prep-TLC (eluent: DCM/MeOH=15/1) to give N-(6-(((6-cyclopropylimidazo[1,2-a]pyridin-2-yl)methyl)amino)pyrimidin-4-yl)-2-(5-methoxy-2-oxopyridin-1 (2H)-yl)acetamide (35 mg, yield: 29%) as a white solid. ESI-MS [M+H]⁺: 446.2. ¹H NMR (400 MHz, DMSO) δ10.66 (s, 1H), 8.29 (s, 1H), 8.23 (s, 1H), 7.89 (s, 1H), 7.60 (s, 1H), 7.37-7.32 (m, 3H), 7.17 (s, 1H), 6.96-6.94 (m, 1H), 6.39-6.36 (m, 1H), 4.71 (s, 2H), 4.57 (s, 2H), 3.62 (s, 3H), 1.92-1.86 (m, 1H), 0.92-0.88 (m, 2H), 0.67-0.63 (m, 2H).

### Example 122

### Synthesis of N-(6-(((6-cyclopropylimidazo[1,2-a]pyridin-2-yl)methyl)amino)pyrimidin-4-yl)-2-(5-methoxy-2-oxobenzo[d]oxazol-3 (2H)yl)acetamide (I-122)

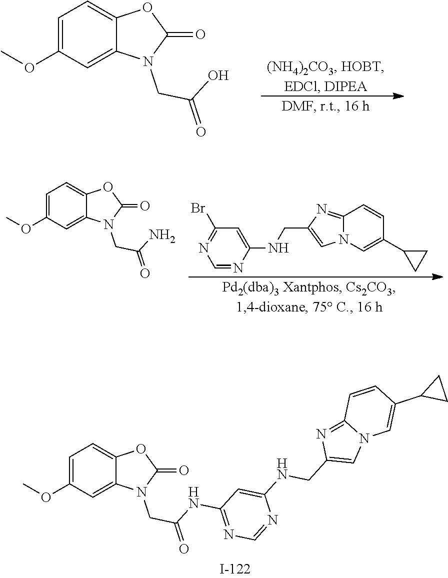 [SMILES: COc1ccc2oc(=O)n(CC(=O)O)c2c1 ; COc1ccc2oc(=O)n(CC(N)=O)c2c1 ; Brc1cc(NCc2cn3cc(C4CC4)ccc3n2)ncn1 ; COc1ccc2oc(=O)n(CC(=O)Nc3cc(NCc4cn5cc(C6CC6)ccc5n4)ncn3)c2c1]

Synthesis of 2-(5-methoxy-2-oxobenzo[d]oxazol-3 (2H)-yl)acetamide. A mixture of 2-(5-methoxy-2-oxobenzo[d]oxazol-3 (2H)-yl)acetic acid (600 mg, 2.7 mmol) (NH₄)₂CO₃ (1.3 g, 13.5 mmol), HOBT (729 mg, 5.4 mmol), EDCI (1.0 g, 5.4 mmol) and DIPEA (1.74 g, 13.5 mmol) in DMF (10 mL) was stirred at room temperature for 16 h. The reaction mixture was quenched with water (100 mL) and extracted with EtOAc (50 mL×3). The combined organic layers were washed with brine (50 mL), dried over anhydrous Na₂SO₄ and concentrated to give the crude, which was purified by column chromatography (eluent: DCM/MeOH=20/1) to give 2-(5-methoxy-2-oxobenzo[d]oxazol-3 (2H)-yl)acetamide (400 mg, yield: 67%) as a yellow oil. ESI-MS [M+H]⁺: 223.2.

Synthesis of N-(6-(((6-cyclopropylimidazo[1,2-a]pyridin-2-yl)methyl)amino)pyrimidin-4-yl)-2-(5-methoxy-2-oxobenzo[d]oxazol-3 (2H)-yl)acetamide. To a mixture of 2-(5-methoxy-2-oxobenzo[d]oxazol-3 (2H)-yl)acetamide (222 mg, 1.0 mmol), 6-bromo-N-((6-cyclopropylimidazo[1,2-a]pyridin-2-yl)methyl)pyrimidin-4-amine (343 mg, 1.0 mmol) (synthesis reported in Example 99), and Cs₂CO₃ (978 mg, 3.0 mmol) in 1,4-dioxane (10 mL) was added Pd₂(dba)₃ (92 mg, 0.1 mmol) and Xantphos (116 mg, 0.2 mmol). The mixture was stirred at 75° C. for 16 h under N₂. The reaction mixture was then cooled to room temperature. The reaction mixture was filtered through Celite® and the filter cake was washed with DCM/MeOH (10/1, 30 mL). The filtrate was concentrated to give the crude, which was purified by Prep-TLC (eluent: DCM/MeOH=15/1) to give N-(6-(((6-cyclopropylimidazo[1,2-a]pyridin-2-yl)methyl)amino)pyrimidin-4-yl)-2-(5-methoxy-2-oxobenzo[d]oxazol-3 (2H)-yl)acetamide (22 mg, yield: 5%) as a white solid. ESI-MS [M+H]⁺: 486.2. ¹H NMR (400 MHz, DMSO) δ=10.78 (s, 1H), 8.28 (s, 1H), 8.24 (s, 1H), 7.90 (s, 1H), 7.59 (s, 1H), 7.35 (d, J=9.4, 1H), 7.27 (d, J=8.8, 1H), 7.16 (s, 1H), 7.02 (d, J=2.5, 1H), 6.94 (dd, J=9.3, 1.7, 1H), 6.67 (dd, J=8.8, 2.6, 1H), 4.76 (s, 2H), 4.57 (s, 2H), 3.74 (s, 3H), 1.93-1.86 (m, 1H), 0.93-0.87 (m, 2H), 0.67-0.63 (m, 2H).

### Example 123

### Synthesis of 2-(5-chloro-2-oxobenzo[d]oxazol-3 (2H)-yl)-N-(6-(((6-cyclopropylimidazo[1,2-a]pyridin-2-yl)methyl)amino pyrimidin-4-yl)acetamide (I-123)

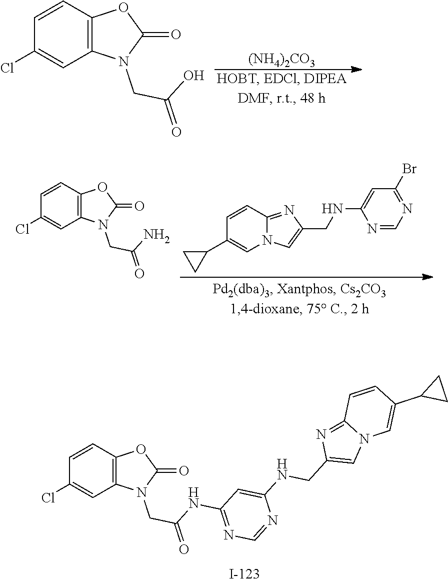 [SMILES: O=C(O)Cn1c(=O)oc2ccc(Cl)cc21 ; NC(=O)Cn1c(=O)oc2ccc(Cl)cc21 ; Brc1cc(NCc2cn3cc(C4CC4)ccc3n2)ncn1 ; O=C(Cn1c(=O)oc2ccc(Cl)cc21)Nc1cc(NCc2cn3cc(C4CC4)ccc3n2)ncn1]

Synthesis of 2-(5-chloro-2-oxobenzo[d]oxazol-3 (2H)-yl)acetamide. A mixture of 2-(5-chloro-2-oxobenzo[d]oxazol-3 (2H)-yl)acetic acid (150 mg, 0.66 mmol), HOBT (135 mg, 1.0 mmol), EDCI (192 mg, 1.0 mmol), DIPEA (258 mg, 2.0 mmol) and (NH₄)₂CO₃ (125 mg, 1.3 mmol) in DMF (10 mL) was stirred at room temperature for 48 h. The reaction mixture was quenched with H₂O (20 mL) and extracted with EtOAc (25 mL×3). The combined organic layers were washed with brine, dried over Na₂SO₄, and concentrated in vacuo. The residue was purified by Prep-TLC, eluting with 50% EtOAc in PE to give 2-(5-chloro-2-oxobenzo[d]oxazol-3 (2H)-yl)acetamide (50 mg, yield: 33%) as a white solid. ESI-MS [M+H]⁺: 227.0.

Synthesis of 2-(5-chloro-2-oxobenzo[d]oxazol-3 (2H)-yl)-N-(6-(((6-cyclopropylimidazo[1,2-a]pyridin-2-yl)methyl)amino)pyrimidin-4-yl)acetamide. A mixture of 2-(5-chloro-2-oxobenzo[d]oxazol-3 (2H)-yl)acetamide (50 mg, 0.22 mmol), 6-bromo-N-((6-cyclopropylimidazo[1,2-a]pyridin-2-yl)methyl)pyrimidin-4-amine (76 mg, 0.22 mmol) (synthesis reported in Example 99), Cs₂CO₃ (215 mg, 0.66 mmol), Pd₂(dba)₃ (18 mg, 0.02 mmol) and Xantphos (23 mg, 0.04 mmol) in 1,4-dioxane (4.0 mL) was stirred at 75° C. for 2 h under N₂ atmosphere. The reaction mixture was cooled to room temperature and filtered through Celite®. The filter cake was washed with DCM/MeOH (V/V=10/1, 20 mL). The filtrate was concentrated in vacuo. The residue was purified by Prep-TLC, eluting with 5% MeOH in DCM to give 2-(5-chloro-2-oxobenzo[d]oxazol-3 (2H)-yl)-N-(6-(((6-cyclopropylimidazo[1,2-a]pyridin-2-yl)methyl)amino)pyrimidin-4-yl)acetamide (8.3 mg, yield: 8%) as a white solid.

ESI-MS [M+H]+: 490.1. 1H NMR (400 MHz, DMSO) δ10.79 (s, 1H), 8.26 (d, J=14.8 Hz, 2H), 7.89 (s, 1H), 7.59 (s, 1H), 7.54 (d, J=2.1 Hz, 1H), 7.41 (d, J=8.6 Hz, 1H), 7.35 (d, J=9.2 Hz, 1H), 7.24-7.11 (m, 2H), 6.94 (dd, J=9.3, 1.8 Hz, 1H), 4.78 (s, 2H), 4.57 (s, 2H), 1.96-1.83 (m, 1H), 0.97-0.85 (m, 2H), 0.65 (dt, J=6.4, 4.4 Hz, 2H).

### Example 124

### Synthesis of 2-(5-chloro-2-oxobenzo[d]oxazol-3 (2H)-yl)-N-(4-(((6-cyclopropylimidazo[1,2-a]pyridin-2-yl)methyl)amino)pyridin-2-yl)acetamide (I-124)

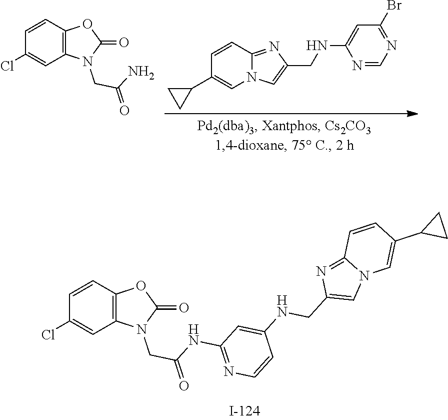 [SMILES: NC(=O)Cn1c(=O)oc2ccc(Cl)cc21 ; Brc1cc(NCc2cn3cc(C4CC4)ccc3n2)ncn1 ; O=C(Cn1c(=O)oc2ccc(Cl)cc21)Nc1cc(NCc2cn3cc(C4CC4)ccc3n2)ccn1]

A mixture of 2-(5-chloro-2-oxobenzo[d]oxazol-3 (2H)-yl)acetamide (60 mg, 0.26 mmol) (synthesis reported in Example 123), 2-bromo-N-((6-cyclopropylimidazo[1,2-a]pyridin-2-yl)methyl)pyridin-4-amine (89 mg, 0.26 mmol) (synthesis reported in Example 128), Cs₂CO₃ (254 mg, 0.78 mmol), Pd₂(dba)₃ (18 mg, 0.02 mmol) and Xantphos (23 mg, 0.04 mmol) in 1,4-dioxane (4.0 mL) was stirred at 75° C. for 2 h under N₂ atmosphere. The reaction mixture was cooled to room temperature and filtered through Celite®. The filter cake was washed with DCM/MeOH (V/V=10/1, 20 mL). The filtrate was concentrated in vacuo. The residue was purified by Prep-TLC, eluting with 5% MeOH in DCM to give 2-(5-chloro-2-oxobenzo[d]oxazol-3 (2H)-yl)-N-(4-(((6-cyclopropylimidazo[1,2-a]pyridin-2-yl)methyl)amino)pyridin-2-yl)acetamide (2.0 mg, yield: 1.5%) as a white solid. ESI-MS [M+H]⁺: 489.1. ¹H NMR (400 MHz, DMSO) δ10.50 (s, 1H), 8.28 (s, 1H), 7.80 (d, J=5.8 Hz, 1H), 7.60 (s, 1H), 7.52 (d, J=2.1 Hz, 1H), 7.43-7.30 (m, 3H), 7.22-7.17 (m, 1H), 7.12 (s, 1H), 6.98-6.93 (m, 1H), 6.40-6.32 (m, 1H), 4.75 (s, 2H), 4.32 (d, J=5.6 Hz, 2H), 1.94-1.85 (m, 1H), 0.93-0.86 (m, 2H), 0.68-0.60 (m, 2H).

### Example 125

### Synthesis of N-(4-(((6-cyclopropylimidazo[1,2-a]pyridin-2-yl)methyl)amino)pyridin-2-yl)-2-(5-methoxy-2-oxobenzo[d]oxazol-3 (2H)-yl)acetamide (I-125)

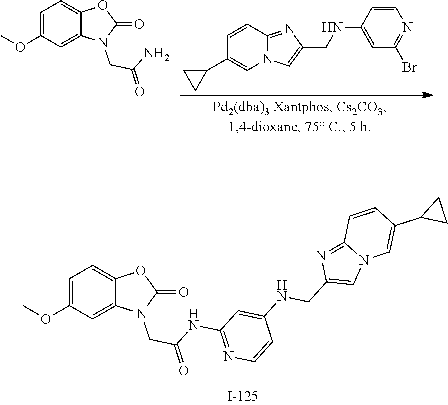 [SMILES: COc1ccc2oc(=O)n(CC(N)=O)c2c1 ; Brc1cc(NCc2cn3cc(C4CC4)ccc3n2)ccn1 ; COc1ccc2oc(=O)n(CC(=O)Nc3cc(NCc4cn5cc(C6CC6)ccc5n4)ccn3)c2c1]

To a mixture of 2-(5-methoxy-2-oxobenzo[d]oxazol-3 (2H)-yl)acetamide (120 mg, 0.54 mmol) (synthesis reported in Example 122), 2-bromo-N-((6-cyclopropylimidazo[1,2-a]pyridin-2-yl)methyl)pyridin-4-amine (185 mg, 0.54 mmol) (synthesis reported in Example 128) and Cs₂CO₃ (528 mg, 1.62 mmol) in 1,4-dioxane (5 mL) was added Pd₂(dba)₃ (46 mg, 0.05 mmol) and Xantphos (58 mg, 0.10 mmol). The mixture was stirred at 75° C. for 5 h under N₂. The reaction mixture was then cooled to room temperature. The reaction mixture was filtered through Celite® and the filter cake was washed with DCM/MeOH (10/1, 20 mL). The filtrate was concentrated to give the crude, which was purified by Prep-TLC (eluent: DCM/MeOH=15/1) to give N-(4-(((6-cyclopropylimidazo[1,2-a]pyridin-2-yl)methyl)amino)pyridin-2-yl)-2-(5-methoxy-2-oxobenzo[d]oxazol-3 (2H)-yl)acetamide (4.5 mg, yield: 2%) as a white solid. ESI-MS [M+H]+: 485.2. ¹H NMR (400 MHz, DMSO) δ10.48 (s, 1H), 8.28 (s, 1H), 7.80 (d, J=5.8 Hz, 1H), 7.61 (s, 1H), 7.38-7.33 (m, 2H), 7.26 (d, J=8.8 Hz, 1H), 7.12 (s, 1H), 6.99 (d, J=2.5 Hz, 1H), 6.95 (dd, J=9.3, 1.7 Hz, 1H), 6.67 (dd, J=8.8, 2.6 Hz, 1H), 6.36 (dd, J=5.8, 2.0 Hz, 1H), 4.72 (s, 2H), 4.32 (d, J=5.7 Hz, 2H), 3.74 (s, 3H), 1.93-1.85 (m, 1H), 0.91-0.84 (m, 2H), 0.67-0.63 (m, 2H).

### Example 126

### Synthesis of 2-(1-(3-chlorophenyl)cyclopropyl)-N-(4-(((6-cyclopropylimidazo[1,2-a]pyridin-2-yl)methyl)amino)pyridin-2-yl)acetamide (I-126)

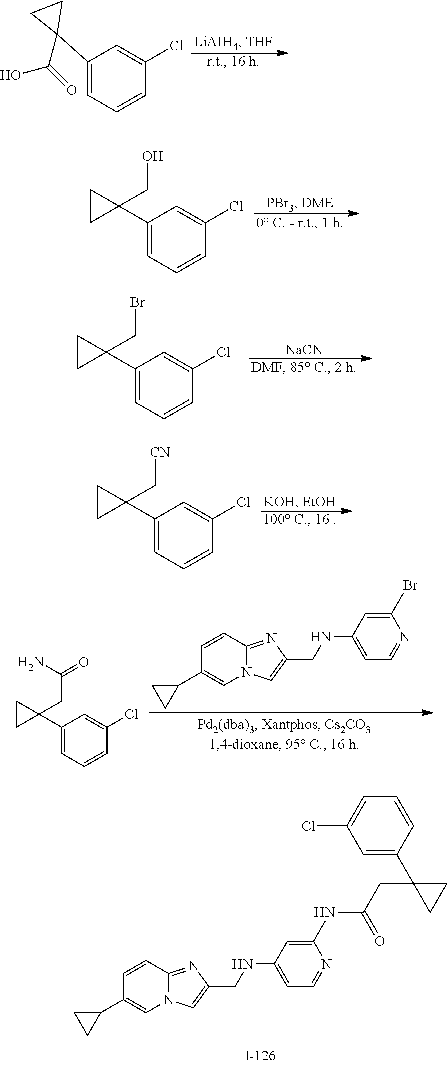

Synthesis of (1-(3-chlorophenyl)cyclopropyl)methanol. To a solution of 1-(3-chlorophenyl)cyclopropane-1-carboxylic acid (1.8 g, 9.2 mmol) in THF (40 mL) was added LiAlH₄ (1.05 g, 27.6 mmol) at 0° C. After the reaction mixture was stirred at room temperature for 16 h under N₂, the reaction was quenched with NH₄Cl (sat. aq., 50 mL) and extracted with EtOAc (50 mL×3). The combined organic layers were washed with brine (30 mL), dried over anhydrous Na₂SO₄, and concentrated in vacuo to give the crude, which was purified by column chromatography (eluent: DCM/MeOH=20/1) to give (1-(3-chlorophenyl)cyclopropyl)methanol (1.4 g, yield: 84%) as a yellow oil. ESI-MS [M+H]+: 183.2.

Synthesis of 1-(1-(bromomethyl)cyclopropyl)-3-chlorobenzene. To a solution of (1-(3-chlorophenyl)cyclopropyl)methanol (600 mg, 3.3 mmol) in DME (20 mL) was added PBr₃ (1.78 g, 6.6 mmol) at 0° C. The mixture was stirred at room temperature for 1 h then quenched with NH₄Cl (sat. aq., 50 mL) and extracted with EtOAc (30 mL×3). The combined organic layers were washed with brine (50 mL), dried over anhydrous Na₂SO₄, and concentrated in vacuo to give the crude, which was purified by column chromatography (eluent: PE/EtOAc=20/1) to give 1-(1-(bromomethyl)cyclopropyl)-3-chlorobenzene (400 mg, yield: 50%) as a yellow oil. ESI-MS [M+H]+: 245.1.

Synthesis of 2-(1-(3-chlorophenyl)cyclopropyl)acetonitrile. A mixture of 1-(1-(bromomethyl)cyclopropyl)-3-chlorobenzene (123 mg, 0.5 mmol) and NaCN (49 mg, 1.0 mmol) in DMF (5 mL) was stirred at 85° C. for 2 h. After the reaction mixture was cooled to room temperature, water (50 mL) was added and extracted with EtOAc (30 mL×3). The combined organic layers were washed with brine (30 mL), dried over anhydrous Na₂SO₄, and concentrated in vacuo to give the crude, which was purified by column chromatography (eluent: PE/EtOAc=5/1) to give 2-(1-(3-chlorophenyl)cyclopropyl)acetonitrile (80 mg, yield: 84%) as a white solid. ESI-MS [M+H]⁺: 192.1.

Synthesis of 2-(1-(3-chlorophenyl)cyclopropyl)acetamide. A mixture of 2-(1-(3-chlorophenyl)cyclopropyl)acetonitrile (80 mg, 0.42 mmol) and KOH (118 mg, 2.1 mmol) in EtOH (5 mL) was stirred at 100° C. for 16 h. The reaction mixture was cooled to room temperature and was quenched with water (30 mL), extracted with EtOAc (30 mL×3). The combined organic layers were washed with brine (30 mL), dried over anhydrous Na₂SO₄, and concentrated in vacuo to give the crude, which was purified by column chromatography (eluent: PE/EA=5/1) to give 2-(1-(3-chlorophenyl)cyclopropyl)acetamide (21 mg, yield: 24%) as a white solid. ESI-MS [M+H]+: 210.1.

Synthesis of 2-(1-(3-chlorophenyl)cyclopropyl)-N-(4-(((6-cyclopropylimidazo[1,2-a]pyridin-2-yl)methyl)amino)pyridin-2-yl)acetamide. A mixture of 2-bromo-N-((6-cyclopropylimidazo[1,2-a]pyridin-2-yl)methyl)pyridin-4-amine (34 mg, 0.1 mmol) (synthesis reported in Example 128), 2-(1-(3-chlorophenyl)cyclopropyl)acetamide (21 mg, 0.1 mmol), Pd₂(dba)₃ (9 mg, 0.01 mmol), Xantphos (12 mg, 0.02 mmol) and Cs₂CO₃ (98 mg, 0.3 mmol) in 1,4-dioxane (5 mL) was stirred at 95° C. for 16 h under N₂. The reaction mixture was cooled to room temperature. The mixture was filtered through Celite® and the filter cake was washed with DCM/MeOH (10/1, 30 mL). The filtrate was concentrated to give the crude, which was purified by Prep-TLC (eluent: DCM/MeOH=15/1) to give 2-(1-(3-chlorophenyl)cyclopropyl)-N-(4-(((6-cyclopropylimidazo[1,2-a]pyridin-2-yl)methyl)amino)pyridin-2-yl)acetamide (6 mg, yield: 13%) as a white solid. ESI-MS [M+H]⁺: 472.1. ¹H NMR (400 MHz, CDCl₃) δ8.25 (s, 1H), 7.84 (s, 1H), 7.77 (d, J=5.9 Hz, 1H), 7.55 (s, 1H), 7.43 (d, J=9.2 Hz, 2H), 7.31 (s, 1H), 7.25-7.10 (m, 3H), 6.96-6.90 (m, 1H), 6.38-6.16 (m, 1H), 5.12 (s, 1H), 4.50 (d, J=5.3 Hz, 2H), 2.69 (s, 2H), 1.92-0.88 (m, 1H), 1.11-0.87 (m, 6H), 0.77-0.53 (m, 2H).

### Example 127

### Synthesis of 2-(3-chloro-1-(4-(((6-cyclopropylimidazo[1,2-a]pyridin-2-yl)methyl)amino)pyridin-2-yl)-6-fluoro-1H-indol-5-yl)acetamide (I-127)

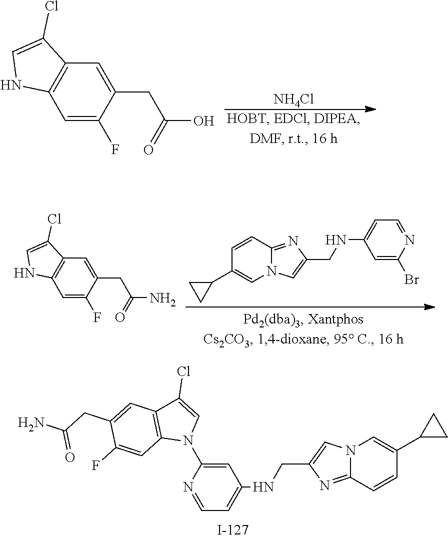 [SMILES: O=C(O)Cc1cc2c(Cl)c[nH]c2cc1F ; NC(=O)Cc1cc2c(Cl)c[nH]c2cc1F ; Brc1cc(NCc2cn3cc(C4CC4)ccc3n2)ccn1 ; NC(=O)Cc1cc2c(Cl)cn(-c3cc(NCc4cn5cc(C6CC6)ccc5n4)ccn3)c2cc1F]

Synthesis of 2-(3-chloro-6-fluoro-1H-indol-5-yl)acetamide. A mixture of 2-(3-chloro-6-2 fluoro-1H-indol-5-yl)acetic acid (227 mg, 1.0 mmol), NH₄Cl (318 mg, 6.0 mol), HOBT (270 mg, 2.0 mmol), EDCI (384 mg, 2.0 mmol) and DIPEA (774 mg, 6.0 mmol) in DMF (10 mL) was stirred at room temperature for 16 h under N₂. The reaction mixture was quenched with H₂O (50 mL) and extracted with EtOAc (50 mL×3). The combined organic layers were washed with brine (50 mL), dried over anhydrous Na₂SO₄, and concentrated to give the crude, which was purified by Prep-TLC (eluent: DCM/MeOH=10/1) to give 2-(3-chloro-6-fluoro-1H-indol-5-yl)acetamide (95 mg, yield: 42%) as a yellow solid. ESI-MS [M+H]+: 227.1.

Synthesis of 2-(3-chloro-1-(4-(((6-cyclopropylimidazo[1,2-a]pyridin-2-yl)methyl)amino)pyridin-2-yl)-6-fluoro-1H-indol-5-yl)acetamide. To a mixture of 2-(3-chloro-6-fluoro-1H-indol-5-yl)acetamide (45 mg, 0.2 mmol), 2-bromo-N-((6-cyclopropylimidazo[1,2-a]pyridin-2-yl)methyl)pyridin-4-amine (68 mg, 0.2 mmol) (synthesis reported in Example 128) and Cs₂CO₃ (196 mg, 0.6 mmol) in 1,4-dioxane (5 mL) was added Pd₂(dba)₃ (18 mg, 0.02 mmol) and Xantphos (23 mg, 0.04 mmol). The reaction was stirred at 95° C. under N₂ for 16 h. The reaction mixture was cooled to room temperature. The mixture was filtered through Celite® and the filter cake was washed with DCM/MeOH (10/1, 30 mL). The filtrate was concentrated to give the crude, which was purified by Prep-TLC (eluent: DCM/MeOH=10/1) to give 2-(3-chloro-1-(4-(((6-cyclopropylimidazo[1,2-a]pyridin-2-yl)methyl)amino)pyridin-2-yl)-6-fluoro-1H-indol-5-yl)acetamide (13.1 mg, yield: 13.5%) as a white solid. ESI-MS [M+H]+: 489.1. ¹H NMR (400 MHz, DMSO) δ8.33 (s, 1H), 8.17-8.12 (m, 2H), 8.00 (d, J=5.8 Hz, 1H), 7.74 (s, 1H), 7.52-7.46 (m, 2H), 7.40 (d, J=9.3 Hz, 1H), 7.36-7.31 (m, 1H), 6.99-6.96 (m, 2H), 6.89 (s, 1H), 6.59-6.58 (m, 1H), 4.51 (d, J=5.5 Hz, 2H), 3.56 (s, 2H), 1.94-1.87 (m, 1H), 0.93-0.88 (m, 2H), 0.68-0.64 (m, 2H).

### Example 128

### Synthesis of (E)-1-(3-chlorobenzyl)-2-cyano-3-(4-(((6-cyclopropylimidazo[1,2-a]pyridin-2-yl)methyl)amino)pyridin-2-yl)guanidine (I-128)

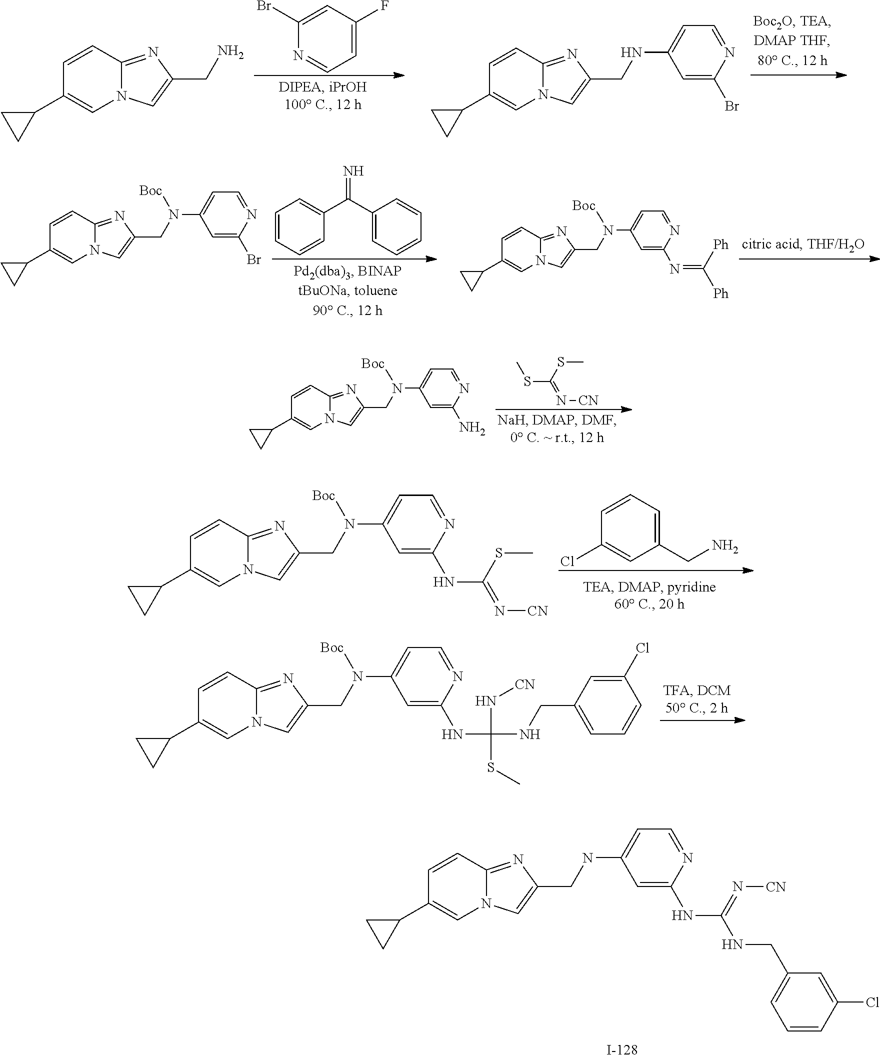

Synthesis of 2-bromo-N-((6-cyclopropylimidazo[1,2-a]pyridin-2-yl)methyl)pyridin-4-amine. A mixture (6-cyclopropylimidazo[1,2-a]pyridin-2-yl)methanamine (935 mg, 5 mmol), 2-bromo-4-fluoropyridine (1.1 g, 6.25 mmol) and DIPEA (1.29 g, 10 mmol) in i-PrOH (30 mL) was stirred at 100° C. for 12 h. The reaction was cooled to room temperature and concentrated in vacuo to give the crude, which was purified with silica gel column chromatography eluting with a gradient of 0-50% EtOAc in PE to give 2-bromo-N-((6-cyclopropylimidazo[1,2-a]pyridin-2-yl)methyl)pyridin-4-amine (1 g, yield: 58%) as a yellow solid. ESI-MS [M+H]⁺: 343.2.

Synthesis of tert-butyl (2-bromopyridin-4-yl)((6-cyclopropylimidazo[1,2-a]pyridin-2-yl)methyl)carbamate. To a solution of 2-bromo-N-((6-cyclopropylimidazo[1,2-a]pyridin-2-yl)methyl)pyridin-4-amine (1 g, 2.9 mmol), TEA (0.88 g, 8.7 mmol) and DMAP (35 mg, 0.29 mmol) in THF (50 mL) was added Boc₂O (0.87 g, 4 mmol). The resulting reaction was stirred at 80° C. for 12 h. The reaction was cooled to room temperature, diluted with H₂O (50 mL) and extracted with EtOAc (60 mL×3). The combined organic layers were washed with brine (80 mL), dried over anhydrous Na₂SO₄, concentrated in vacuo to give the crude, which was purified with silica gel column chromatography eluting with a gradient of 0-30% EtOAc in PE to give tert-butyl (2-bromopyridin-4-yl)((6-cyclopropylimidazo[1,2-a]pyridin-2-yl)methyl)carbamate (740 mg, yield: 58%) as a yellow solid. ESI-MS [M+H]+: 443.2.

Synthesis of tert-butyl ((6-cyclopropylimidazo[1,2-a]pyridin-2-yl)methyl)(2-((diphenylmethylene)amino)pyridin-4-yl)carbamate. A mixture of tert-butyl (2-bromopyridin-4-yl)((6-cyclopropylimidazo[1,2-a]pyridin-2-yl)methyl)carbamate (740 mg, 1.7 mmol), diphenylmethanimine (615 mg, 3.4 mmol), Pd₂(dba)₃ (156 mg, 0.17 mmol), BINAP (211 mg, 0.34 mmol) and tBuONa (490 mg, 5.1 mmol) in toluene (20 mL) was stirred at 90° C. for 12 h under N₂. The reaction was cooled to room temperature, diluted with H₂O (50 mL) and extracted with EtOAc (60 mL×3). The combined organic layers were washed with brine (80 mL), dried over anhydrous Na₂SO₄, concentrated in vacuo to give the crude, which was purified with silica gel column chromatography eluting with a gradient of 0-50% EtOAc in PE to give tert-butyl ((6-cyclopropylimidazo[1,2-a]pyridin-2-yl)methyl)(2-((diphenylmethylene)amino)pyridin-4-yl)carbamate (680 mg, yield: 74%) as a yellow solid. ESI-MS [M+H]⁺: 544.2.

Synthesis of tert-butyl (2-aminopyridin-4-yl)((6-cyclopropylimidazo[1,2-a]pyridin-2-yl)methyl)carbamate. To a solution of tert-butyl ((6-cyclopropylimidazo[1,2-a]pyridin-2-yl)methyl)(2-((diphenylmethylene)amino)pyridin-4-yl)carbamate (680 mg, 1.25 mmol) in THF/H₂O (15 mL/5 mL) was added citric acid (960 mg, 5 mmol). The resulting reaction was stirred at room temperature for 2 h. Then the reaction was washed with NaHCO₃ (sat. aq., 50 mL), extracted with EtOAc (50 mL×3). The combined organic layers were washed with brine (60 mL), dried over anhydrous Na₂SO₄, concentrated in vacuo to give the crude, which was purified with silica gel column chromatography eluting with a gradient of 0-50% EtOAc in PE to give tert-butyl (2-aminopyridin-4-yl)((6-cyclopropylimidazo[1,2-a]pyridin-2-yl)methyl)carbamate (360 mg, yield: 76%) as a yellow solid. ESI-MS [M+H]⁺: 380.3.

Synthesis of tert-butyl (Z)-(2-(((cyanoimino)(methylthio)methyl)amino)pyridin-4-yl)((6-cyclopropylimidazo[1,2-a]pyridin-2-yl)methyl)carbamate. To a solution of tert-butyl (2-aminopyridin-4-yl)((6-cyclopropylimidazo[1,2-a]pyridin-2-yl)methyl)carbamate (360 mg, 0.95 mmol) and dimethyl cyanocarbonimidodithioate (204 mg, 1.4 mmol) in DMF (10 mL) was added DMAP (23 mg, 0.19 mmol) and NaH (76 mg, 1.9 mmol, 60% dispersion in mineral oil) at 0° C. The resulting reaction mixture was stirred at room temperature for 12 h. Then the reaction was quenched with NH₄Cl (sat. aq., 50 mL), extracted with EtOAc (50 mL×3). The combined organic layers were washed with brine (50 mL), dried over anhydrous Na₂SO₄, concentrated in vacuo to give the crude, which was purified with silica gel column chromatography eluting with a gradient of 0-30% EtOAc in PE to give tert-butyl (Z)-(2-(((cyanoimino)(methylthio)methyl)amino)pyridin-4-yl)((6-cyclopropylimidazo[1,2-a]pyridin-2-yl)methyl)carbamate (285 mg, yield: 63%) as a yellow solid. ESI-MS [M+H]⁺: 478.1.

Synthesis of tert-butyl (2-((((3-chlorobenzyl)amino)(cyanamido)(methylthio)methyl)amino)pyridin-4-yl)((6-cyclopropylimidazo[1,2-a]pyridin-2-yl)methyl)carbamate. A mixture of tert-butyl (Z)-(2-(((cyanoimino)(methylthio)methyl)amino)pyridin-4-yl)((6-cyclopropylimidazo[1,2-a]pyridin-2-yl)methyl)carbamate (150 mg, 0.31 mmol), (3-chlorophenyl)methanamine (131 mg, 0.93 mmol), TEA (162 mg, 1.6 mmol) and DMAP (8 mg, 0.062 mmol) in dry pyridine (5 mL) was stirred at 60° C. for 20 h. After cooled to room temperature, the reaction was concentrated in vacuo, the residue was diluted with H₂O (30 mL) and extracted with EtOAc (40 mL×3). The combined organic layers were washed with brine (50 mL), dried over anhydrous Na₂SO₄, concentrated in vacuo to give the crude, which was purified with Prep-TLC (eluent: PE/EtOAc=2/1) to give tert-butyl (2-((((3-chlorobenzyl)amino)(cyanamido)(methylthio)methyl)amino)pyridin-4-yl)((6-cyclopropylimidazo[1,2-a]pyridin-2-yl)methyl)carbamate (85 mg, yield: 44%) as a yellow solid. ESI-MS [M+H]+: 619.2.

Synthesis of (E)-1-(3-chlorobenzyl)-2-cyano-3-(4-(((6-cyclopropylimidazo[1,2-a]pyridin-2-yl)methyl)amino)pyridin-2-yl)guanidine. To a solution of tert-butyl (2-((((3-chlorobenzyl)amino)(cyanamido)(methylthio)methyl)amino)pyridin-4-yl)((6-cyclopropylimidazo[1,2-a]pyridin-2-yl)methyl)carbamate (85 mg, 0.14 mmol) in DCM (5 mL) was added TFA (1 mL). After the reaction solution was stirred at 50° C. for 2 h, the reaction was cooled to room temperature and concentrated in vacuo, the residue was neutralized with NaHCO₃ (sat. aq., 40 mL) and extracted with EtOAc (50 mL×3). The combined organic layers were washed with brine (40 mL), dried over anhydrous Na₂SO₄, concentrated in vacuo to give the crude which was purified with Prep-TLC (eluent: DCM/MeOH=15/1) to give (E)-1-(3-chlorobenzyl)-2-cyano-3-(4-(((6-cyclopropylimidazo[1,2-a]pyridin-2-yl)methyl)amino)pyridin-2-yl)guanidine (19 mg, yield: 29%) as a white solid. ESI-MS [M+H]+: 471.2. ESI-MS [M+H]+: 471.1. ¹H NMR (400 MHz, DMSO) δ8.61-8.30 (m, 3H), 8.09-7.63 (m, 3H), 7.42-7.27 (m, 5H), 7.00 (d, J=9.3 Hz, 1H), 6.69-6.59 (m, J=42.7 Hz, 1H), 6.10-5.91 (m, 1H), 4.50-4.34 (m, J=66.1 Hz, 4H), 1.92 (s, 1H), 1.02-0.85 (m, 2H), 0.67 (d, J=4.5 Hz, 2H).

### Example 129

### Synthesis of 2-(3-chloro-1H-pyrrolo[2,3-b]pyridin-5-yl)-N-(6-(((6-cyclopropylimidazo[1,2-a]pyridin-2-yl)methyl)amino)pyrimidin-4-yl)acetamide (I-129)

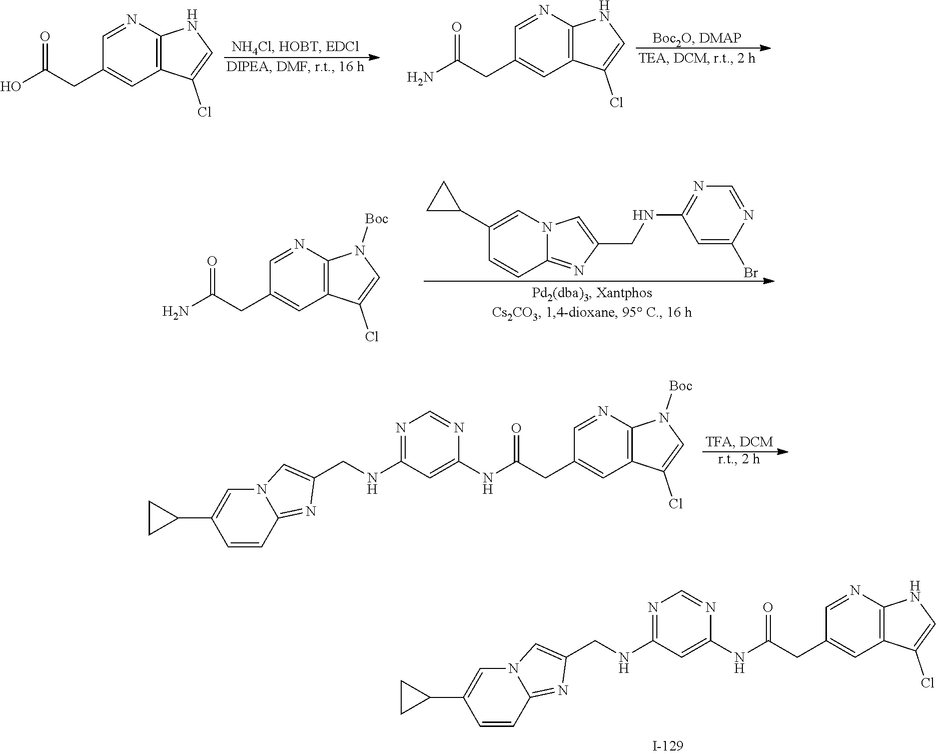 [SMILES: O=C(O)Cc1cnc2[nH]cc(Cl)c2c1 ; NC(=O)Cc1cnc2[nH]cc(Cl)c2c1 ; CC(C)(C)OC(=O)n1cc(Cl)c2cc(CC(N)=O)cnc21 ; Brc1cc(NCc2cn3cc(C4CC4)ccc3n2)ncn1 ; CC(C)(C)OC(=O)n1cc(Cl)c2cc(CC(=O)Nc3cc(NCc4cn5cc(C6CC6)ccc5n4)ncn3)cnc21 ; O=C(Cc1cnc2[nH]cc(Cl)c2c1)Nc1cc(NCc2cn3cc(C4CC4)ccc3n2)ncn1]

Synthesis of 2-(3-chloro-1H-pyrrolo[2,3-b]pyridin-5-yl)acetamide. A mixture of 2-(3-chloro-1H-pyrrolo[2,3-b]pyridin-5-yl)acetic acid (210 mg, 1.0 mmol), NH₄Cl (212 mg, 4.0 mmol), HOBT (270 mg, 2.0 mmol), EDCI (384 mg, 2.0 mmol) and DIPEA (645 mg, 5.0 mmol) in DMF (10 mL) was stirred at room temperature for 16 h under N₂. The reaction was quenched with water (100 mL) and extracted with EtOAc (30 mL×3). The combined organic layers were washed with brine (50 mL), dried over anhydrous Na₂SO₄, and concentrated to give the crude product, which was purified by Prep-TLC (eluent: DCM/MeOH=20/1) to give product 2-(3-chloro-1H-pyrrolo[2,3-b]pyridin-5-yl)acetamide (180 mg, yield: 86%) as a white solid. ESI-MS [M+H]+: 210.1.

Synthesis of tert-butyl 5-(2-amino-2-oxoethyl)-3-chloro-1H-pyrrolo[2,3-b]pyridine-1-carboxylate. A mixture of 2-(3-chloro-1H-pyrrolo[2,3-b]pyridin-5-yl)acetamide (180 mg, 0.86 mmol), DMAP (21 mg, 0.17 mmol), Boc₂O (281 mg, 1.29 mmol) and TEA (261 mg, 2.58 mmol) in DCM (20 mL) was stirred at room temperature for 2 h. The reaction was quenched with water (50 mL) and extracted with DCM (30 mL×3). The combined organic layers were washed with brine (50 mL), dried over anhydrous Na₂SO₄, and concentrated to give the crude product, which was purified by Prep-TLC (eluent: DCM/MeOH=20/1) to give tert-butyl 5-(2-amino-2-oxoethyl)-3-chloro-1H-pyrrolo[2,3-b]pyridine-1-carboxylate (190 mg, yield: 72%) as a white solid. ESI-MS [M+H]+: 310.1.

Synthesis of tert-butyl 3-chloro-5-(2-((6-(((6-cyclopropylimidazo[1,2-a]pyridin-2-yl)methyl)amino)pyrimidin-4-yl)amino)-2-oxoethyl)-1H-pyrrolo[2,3-b]pyridine-1-carboxylate. A mixture of tert-butyl 5-(2-amino-2-oxoethyl)-3-chloro-1H-pyrrolo[2,3-b]pyridine-1-carboxylate (90 mg, 0.29 mmol), 6-bromo-N-((6-cyclopropylimidazo[1,2-a]pyridin-2-yl)methyl)pyrimidin-4-amine (99 mg, 0.29 mmol), Pd₂(dba)₃ (27 mg, 0.03 mmol), Xantphos (35 mg, 0.06 mmol) and Cs₂CO₃ (284 mg, 0.87 mmol) in 1,4-dioxane (10 mL) was stirred at 95° C. for 16 h under N₂. After the reaction mixture was cooled to room temperature, the mixture was filtered through Celite® and the filter cake was washed with DCM/MeOH (10/1, 30 mL). The filtrate was concentrated to give the crude, which was purified by Prep-TLC (eluent: DCM/MeOH=10/1) to give tert-butyl 3-chloro-5-(2-((6-(((6-cyclopropylimidazo[1,2-a]pyridin-2-yl)methyl)amino)pyrimidin-4-yl)amino)-2-oxoethyl)-1H-pyrrolo[2,3-b]pyridine-1-carboxylate (30 mg, yield: 18%) as a white solid. ESI-MS [M+H]+: 573.2.

Synthesis of 2-(3-chloro-1H-pyrrolo[2,3-b]pyridin-5-yl)-N-(6-(((6-cyclopropylimidazo[1,2-a]pyridin-2-yl)methyl)amino)pyrimidin-4-yl)acetamide. To a solution of tert-butyl 3-chloro-5-(2-((6-(((6-cyclopropylimidazo[1,2-a]pyridin-2-yl)methyl)amino)pyrimidin-4-yl)amino)-2-oxoethyl)-1H-pyrrolo[2,3-b]pyridine-1-carboxylate (30 mg, 0.05 mmol) in DCM (5 mL) was added TFA (0.5 mL) at 0° C. After the mixture was stirred at room temperature for 2 h, the reaction was quenched with NaHCO₃ (sat. aq., 30 mL) and extracted with DCM (30 mL×3). The combined organic layers were washed with brine (50 mL), dried over Na₂SO₄, and concentrated in vacuo to give the crude, which was purified by Prep-TLC (eluent: DCM/MeOH=10/1) to give 2-(3-chloro-1H-pyrrolo[2,3-b]pyridin-5-yl)-N-(6-(((6-cyclopropylimidazo[1,2-a]pyridin-2-yl)methyl)amino)pyrimidin-4-yl)acetamide (2 mg, yield: 8%) as a white solid. ESI-MS [M+H]+: 473.1. ¹H NMR (400 MHz, DMSO) δ11.93 (s, 1H), 10.59 (s, 1H), 8.34-8.18 (m, 3H), 7.88 (s, 1H), 7.66 (s, 1H), 7.58 (s, 1H), 7.35 (d, J=9.3 Hz, 1H), 7.23 (s, 1H), 6.98-6.91 (m, 1H), 4.55 (s, 2H), 3.82 (s, 2H), 1.95-1.82 (m, 1H), 0.95-0.79 (m, 2H), 0.72-0.59 (m, 2H).

### Example 130

### Synthesis of 2-(3-chloro-1H-pyrrolo[2,3-b]pyridin-5-yl)-N-(4-(((6-cyclopropylimidazo[1,2-a]pyridin-2-yl)methyl)amino)pyridin-2-yl)acetamide (I-130)

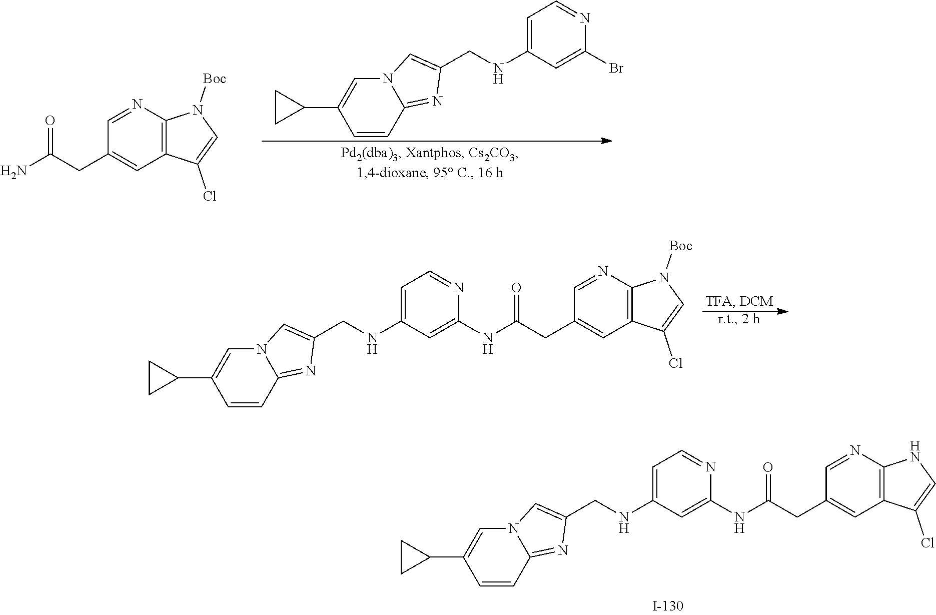 [SMILES: CC(C)(C)OC(=O)n1cc(Cl)c2cc(CC(N)=O)cnc21 ; Brc1cc(NCc2cn3cc(C4CC4)ccc3n2)ccn1 ; CC(C)(C)OC(=O)n1cc(Cl)c2cc(CC(=O)Nc3cc(NCc4cn5cc(C6CC6)ccc5n4)ccn3)cnc21 ; O=C(Cc1cnc2[nH]cc(Cl)c2c1)Nc1cc(NCc2cn3cc(C4CC4)ccc3n2)ccn1]

Synthesis of tert-butyl 3-chloro-5-(2-((4-(((6-cyclopropylimidazo[1,2-a]pyridin-2-yl)methyl)amino)pyridin-2-yl)amino)-2-oxoethyl)-1H-pyrrolo[2,3-b]pyridine-1-carboxylate. A mixture of tert-butyl 5-(2-amino-2-oxoethyl)-3-chloro-1H-pyrrolo[2,3-b]pyridine-1-carboxylate (103 mg, 0.33 mmol), 2-bromo-N-((6-cyclopropylimidazo[1,2-a]pyridin-2-yl)methyl)pyridin-4-amine (172 mg, 0.50 mmol), Pd₂(dba)₃ (60 mg, 0.066 mmol), Xantphos (38 mg, 0.066 mmol) and Cs₂CO₃ (323 mg, 0.99 mmol) in 1,4-dioxane (15 mL) was stirred at 95° C. for 16 h under N₂. The mixture was cooled to room temperature and filtered through Celite®. The filter cake was washed with DCM/MeOH (V/V=10/1, 20 mL). The filtrate was concentrated in vacuo. The residue was purified by Prep-TLC, eluting with 10% MeOH in DCM to give tert-butyl 3-chloro-5-(2-((4-(((6-cyclopropylimidazo[1,2-a]pyridin-2-yl)methyl)amino)pyridin-2-yl)amino)-2-oxoethyl)-1H-pyrrolo[2,3-b]pyridine-1-carboxylate (20 mg, yield: 11%) as a white solid. ESI-MS [M+H]+: 572.2.

Synthesis of 2-(3-chloro-1H-pyrrolo[2,3-b]pyridin-5-yl)-N-(4-(((6-cyclopropylimidazo[1,2-a]pyridin-2-yl)methyl)amino)pyridin-2-yl)acetamide. TFA (0.5 mL) was added to a solution of tert-butyl 3-chloro-5-(2-((4-(((6-cyclopropylimidazo[1,2-a]pyridin-2-yl)methyl)amino)pyridin-2-yl)amino)-2-oxoethyl)-1H-pyrrolo[2,3-b]pyridine-1-carboxylate (20 mg, 0.035 mmol)) in DCM (5.0 mL) at 0° C. The resulting mixture was stirred at room temperature for 2 h. The reaction was quenched with NaHCO₃ (sat. aq., 20 mL) and extracted with EtOAc (30 mL×3). The combined organic layers were washed with brine (20 mL), dried over Na₂SO₄, and then concentrated in vacuo. The residue was purified by Prep-TLC, eluting with 5% MeOH in DCM to give 2-(3-chloro-1H-pyrrolo[2,3-b]pyridin-5-yl)-N-(4-(((6-cyclopropylimidazo[1,2-a]pyridin-2-yl)methyl)amino)pyridin-2-yl)acetamide (5.0 mg, yield: 30%) as a white solid. ESI-MS [M+H]+: 472.1. 1H NMR (400 MHz, DMSO) δ11.95 (s, 1H), 10.29 (s, 1H), 8.30-8.22 (m, 2H), 7.88 (d, J=1.8 Hz, 1H), 7.76 (d, J=5.8 Hz, 1H), 7.66 (s, 1H), 7.60 (s, 1H), 7.43-7.31 (m, 2H), 7.10 (t, J=5.7 Hz, 1H), 7.00-6.91 (m, 1H), 6.36-6.28 (m, 1H), 4.32 (d, J=5.6 Hz, 2H), 3.78 (s, 2H), 1.95-1.82 (m, 1H), 0.98-0.81 (m, 2H), 0.71-0.59 (m, 2H).

### Example 131

### Synthesis of 2-(3-chloro-6-fluoro-1H-indol-5-yl)-N-(6-(((6-cyclopropylimidazo[1,2-a]pyridin-2-yl)methyl)amino)pyrimidin-4-yl)acetamide (I-131)

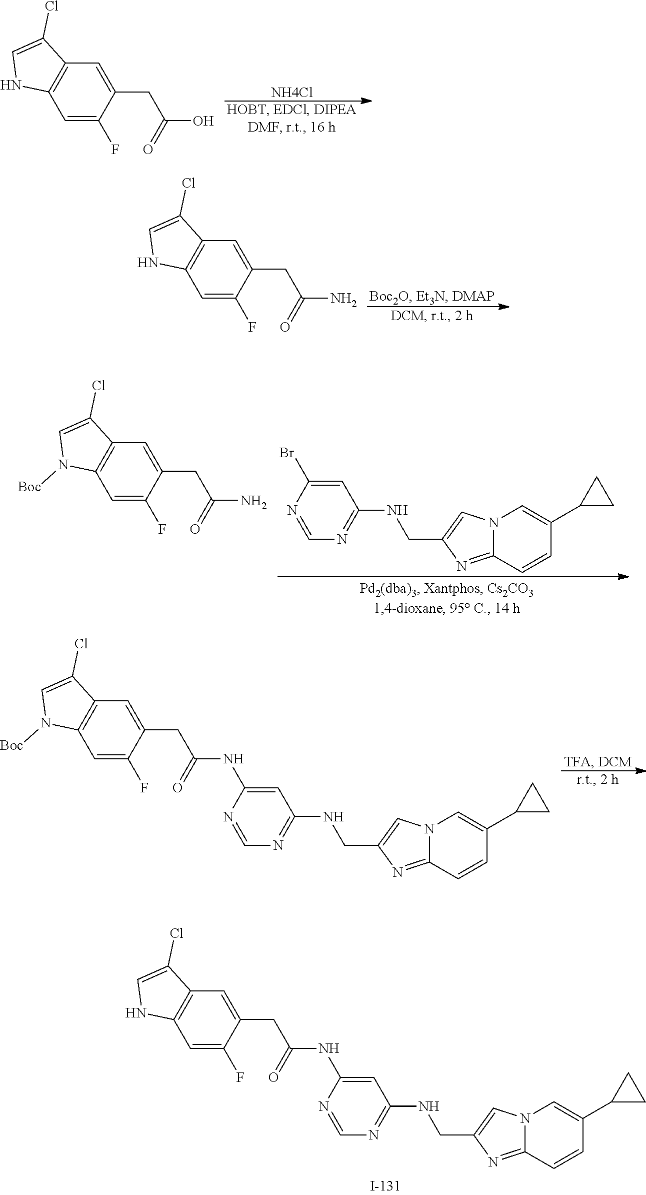 [SMILES: O=C(O)Cc1cc2c(Cl)c[nH]c2cc1F ; NC(=O)Cc1cc2c(Cl)c[nH]c2cc1F ; CC(C)(C)OC(=O)n1cc(Cl)c2cc(CC(N)=O)c(F)cc21 ; Brc1cc(NCc2cn3cc(C4CC4)ccc3n2)ncn1 ; CC(C)(C)OC(=O)n1cc(Cl)c2cc(CC(=O)Nc3cc(NCc4cn5cc(C6CC6)ccc5n4)ncn3)c(F)cc21 ; O=C(Cc1cc2c(Cl)c[nH]c2cc1F)Nc1cc(NCc2cn3cc(C4CC4)ccc3n2)ncn1]

Synthesis of 2-(3-chloro-6-fluoro-1H-indol-5-yl)acetamide. A mixture of 2-(3-chloro-6-fluoro-1H-indol-5-yl)acetic acid (150 mg, 0.66 mmol), NH₄Cl (210 mg, 3.96 mol), DIPEA (511 mg, 4.0 mmol), HOBT (178 mg, 1.32 mmol) and EDCI (252 mg, 1.32 mmol) in DMF (10 mL) was stirred at room temperature for 16 h. H₂O (30 mL) was added and extracted with EtOAc (30 mL×3). The combined organics were washed with brine (30 mL), dried over Na₂SO₄, and concentrated in vacuo. The residue was purified by Prep-TLC, eluting with 3% MeOH in DCM to give 2-(3-chloro-6-fluoro-1H-indol-5-yl)acetamide (115 mg, yield: 77%) as yellow solid. ESI-MS [M+H]+: 227.1.

Synthesis of tert-butyl 5-(2-amino-2-oxoethyl)-3-chloro-6-fluoro-1H-indole-1-carboxylate. To a solution of 2-(3-chloro-6-fluoro-1H-indol-5-yl)acetamide (115 mg, 0.51 mol) in DCM (15 mL) was added DMAP (12 mg, 0.098 mmol), Et₃N (155 mg, 1.53 mmol) and Boc₂O (122 mg, 0.56 mol) at 0° C. The resulting mixture was stirred at room temperature for 2 h. H₂O (30 mL) was added and extracted with EtOAc (30 mL×3). The combined organics were washed with brine (30 mL), dried over Na₂SO₄, and concentrated in vacuo. The residue was purified by Prep-TLC, eluting with 3% MeOH in DCM to give tert-butyl 5-(2-amino-2-oxoethyl)-3-chloro-6-fluoro-1H-indole-1-carboxylate (140 mg, yield: 84%) as a yellow solid. ESI-MS [M+H]+: 327.1.

Synthesis of tert-butyl 3-chloro-5-(2-((6-(((6-cyclopropylimidazo[1,2-a]pyridin-2-yl)methyl)amino)pyrimidin-4-yl)amino)-2-oxoethyl)-6-fluoro-1H-indole-1-carboxylate. A mixture of tert-butyl 5-(2-amino-2-oxoethyl)-3-chloro-6-fluoro-1H-indole-1-carboxylate (50 mg, 0.15 mmol), 6-bromo-N-((6-cyclopropylimidazo[1,2-a]pyridin-2-yl)methyl)pyrimidin-4-amine (62 mg, 0.18 mmol), Cs₂CO₃ (147 mg, 0.45 mmol), Xantphos (17 mg, 0.029 mmol) and Pd₂(dba)₃ (14 mg, 0.015 mmol) in 1,4-dioxane (6.0 mL) was stirred at 95° C. for 14 h under N₂ atmosphere. After cooling to room temperature, H₂O (20 mL) was added and then extracted with EtOAc (30 mL×3). The combined organics were washed with brine (30 mL), dried over Na₂SO₄, and concentrated in vacuo. The residue was purified by Prep-TLC, eluting with 5% MeOH in DCM to give tert-butyl 3-chloro-5-(2-((6-(((6-cyclopropylimidazo[1,2-a]pyridin-2-yl)methyl)amino)pyrimidin-4-yl)amino)-2-oxoethyl)-6-fluoro-1H-indole-1-carboxylate (25 mg, yield: 28%) as a white solid. ESI-MS [M+H]⁺: 590.1.

Synthesis of 2-(3-chloro-6-fluoro-1H-indol-5-yl)-N-(6-(((6-Cyclopropylimidazo[1,2-a]pyridin-2-yl)methyl)amino)pyrimidin-4-yl)acetamide. To a solution of tert-butyl 3-chloro-5-(2-((6-(((6-cyclopropylimidazo[1,2-a]pyridin-2-yl)methyl)amino)pyrimidin-4-yl)amino)-2-oxoethyl)-6-fluoro-1H-indole-1-carboxylate (25 mg, 0.042 mmol) in DCM (5.0 mL) was added TFA (0.5 mL). The mixture was stirred at room temperature for 2 h. The resulting mixture was neutralized with NaHCO₃ (sat. aq., 10 mL) and then extracted with EtOAc (30 mL×3). The combined organics were concentrated in vacuo. The residue was purified by Prep-TLC, eluting with 10% MeOH in DCM to give 2-(3-chloro-6-fluoro-1H-indol-5-yl)-N-(6-(((6-cyclopropylimidazo[1,2-a]pyridin-2-yl)methyl)amino)pyrimidin-4-yl)acetamide (12.2 mg, yield: 59%) as a white solid. ESI-MS [M+H]+: 490.2. 1H NMR (400 MHz, DMSO) δ11.38 (s, 1H), 10.50 (s, 1H), 8.28 (s, 1H), 8.21 (s, 1H), 7.85 (s, 1H), 7.59 (s, 1H), 7.51 (s, 1H), 7.45 (d, J=6.9 Hz, 1H), 7.35 (d, J=9.3 Hz, 1H), 7.22 (s, 2H), 6.94 (d, J=9.3 Hz, 1H), 4.56 (s, 2H), 3.85 (s, 2H), 1.93-1.86 (m, 1H), 0.92-0.87 (m, 2H), 0.67-0.64 (m, 2H).

### Example 132

### Synthesis of 2-(3-chloro-6-fluoro-1H-indol-5-yl)-N-(4-(((6-cyclopropylimidazo[1,2-a]pyridin-2-yl)methyl)amino)pyridin-2-yl)acetamide (I-132)

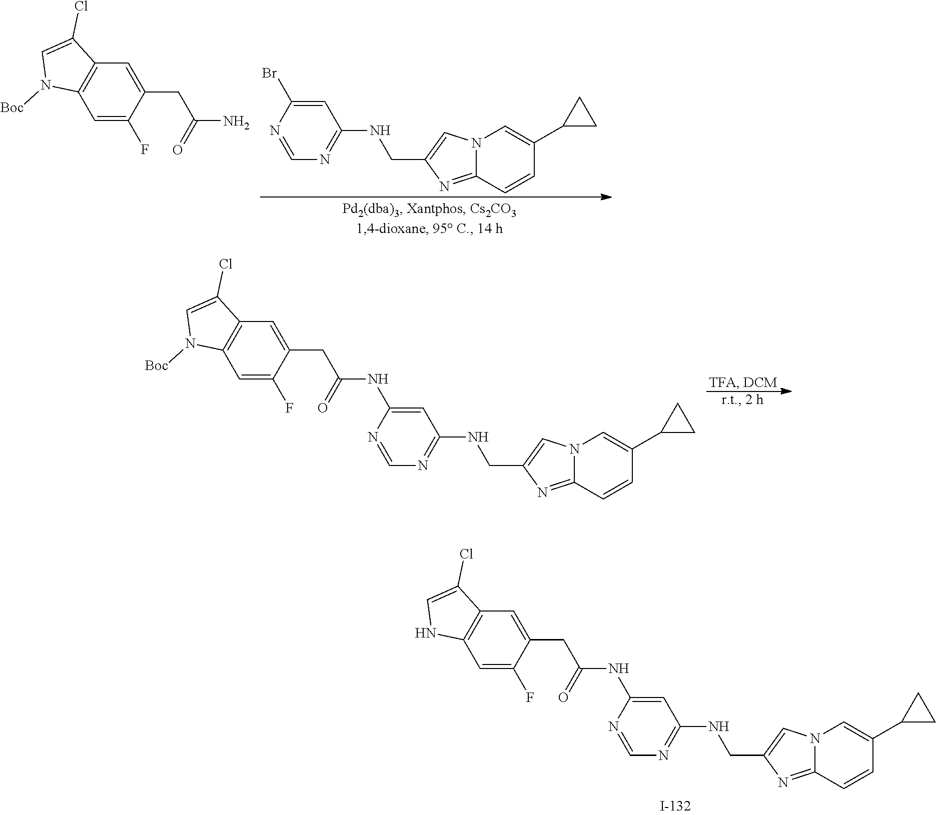 [SMILES: CC(C)(C)OC(=O)n1cc(Cl)c2cc(CC(N)=O)c(F)cc21 ; Brc1cc(NCc2cn3cc(C4CC4)ccc3n2)ncn1 ; CC(C)(C)OC(=O)n1cc(Cl)c2cc(CC(=O)Nc3cc(NCc4cn5cc(C6CC6)ccc5n4)ncn3)c(F)cc21 ; O=C(Cc1cc2c(Cl)c[nH]c2cc1F)Nc1cc(NCc2cn3cc(C4CC4)ccc3n2)ncn1]

Synthesis of tert-butyl 3-chloro-5-(2-((4-(((6-cyclopropylimidazo[1,2-a]pyridin-2-yl)methyl)amino)pyridin-2-yl)amino)-2-oxoethyl)-6-fluoro-1H-indole-1-carboxylate. A mixture of tert-butyl 5-(2-amino-2-oxoethyl)-3-chloro-6-fluoro-1H-indole-1-carboxylate (50 mg, 0.15 mmol), 2-bromo-N-((6-cyclopropylimidazo[1,2-a]pyridin-2-yl)methyl)pyridin-4-amine (62 mg, 0.18 mmol), Cs₂CO₃ (147 mg, 0.45 mmol), Xantphos (17 mg, 0.029 mmol) and Pd₂(dba)₃ (14 mg, 0.015 mmol) in 1,4-dioxane (6.0 mL) was stirred at 95° C. for 14 h under N₂ atmosphere. After cooling to room temperature, H₂O (20 mL) was added and then extracted with EtOAc (20 mL×3). The combined organics were washed with brine (30 mL), dried over Na₂SO₄, and concentrated in vacuo. The residue was purified by Prep-TLC, eluting with 5% MeOH in DCM to give tert-butyl 3-chloro-5-(2-((4-(((6-cyclopropylimidazo[1,2-a]pyridin-2-yl)methyl)amino)pyridin-2-yl)amino)-2-oxoethyl)-6-fluoro-1H-indole-1-carboxylate (40 mg, yield: 45%) as white solid. ESI-MS [M+H]⁺: 589.2.

Synthesis of 2-(3-chloro-6-fluoro-1H-indol-5-yl)-N-(4-(((6-cyclopropylimidazo[1,2-a]pyridin-2-yl)methyl)amino)pyridin-2-yl)acetamide. To a solution of tert-butyl 3-chloro-5-(2-((4-(((6-cyclopropylimidazo[1,2-a]pyridin-2-yl)methyl)amino)pyridin-2-yl)amino)-2-oxoethyl)-6-fluoro-1H-indole-1-carboxylate (40 mg, 0.068 mmol) in DCM (5.0 mL) was added TFA (0.5 mL). The mixture was stirred at room temperature for 2 h. The resulting mixture was neutralized with NaHCO₃ (sat. aq., 10 mL) and then extracted with EtOAc (30 mL×3). The combined organics were concentrated in vacuo. The residue was purified by Prep-TLC, eluting with 10% MeOH in DCM to give 2-(3-chloro-6-fluoro-1H-indol-5-yl)-N-(4-(((6-cyclopropylimidazo[1,2-a]pyridin-2-yl)methyl)amino)pyridin-2-yl)acetamide (20.7 mg, yield: 62%) as white solid. ESI-MS [M+H]⁺: 489.1. 1H NMR (400 MHz, DMSO) δ11.36 (s, 1H), 10.17 (s, 1H), 8.28 (s, 1H), 7.77 (d, J=5.8 Hz, 1H), 7.60 (s, 1H), 7.49 (s, 1H), 7.46 (d, J=7.2 Hz, 1H), 7.39 (s, 1H), 7.36 (d, J=9.3 Hz, 1H), 7.19 (d, J=10.6 Hz, 1H), 7.10 (t, J=5.7 Hz, 1H), 6.96-6.94 (m, 1H), 6.33-6.31 (m, 1H), 4.32 (d, J=5.7 Hz, 2H), 3.81 (s, 2H), 1.93-1.86 (m, 1H), 0.92-0.83 (m, 2H), 0.69-0.63 (m, 2H).

### Example 133

### Synthesis of (2S,3R)-3-(3-chlorophenyl)-N-(6-(((6-cyclopropylimidazo[1,2-a]pyridin-2-yl)methyl amino)pyrimidin-4-y I-2-methylbutanamide (I-133)

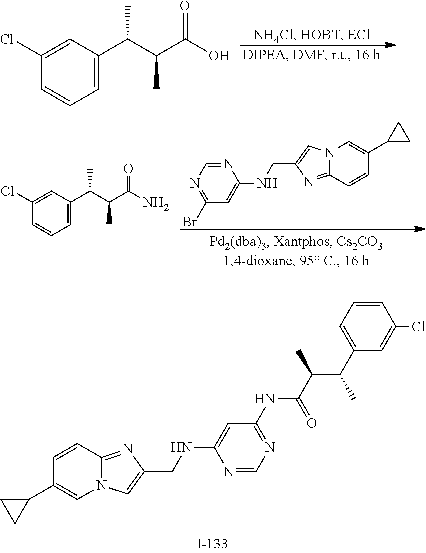 [SMILES: C[C@H](C(=O)O)[C@@H](C)c1cccc(Cl)c1 ; C[C@H](C(N)=O)[C@@H](C)c1cccc(Cl)c1 ; Brc1cc(NCc2cn3cc(C4CC4)ccc3n2)ncn1 ; C[C@H](C(=O)Nc1cc(NCc2cn3cc(C4CC4)ccc3n2)ncn1)[C@@H](C)c1cccc(Cl)c1]

Synthesis of (2S,3R)-3-(3-chlorophenyl)-2-methylbutanamide. A mixture of (2S,3R)-3-(3-chlorophenyl)-2-methylbutanoic acid (83 mg, 0.39 mmol), NH₄Cl (127 mg, 2.4 mmol), HOBT (108 mg, 0.80 mmol), DIPEA (310 mg, 2.4 mmol) and EDCI (153 mg, 0.80 mmol) in DMF (5.0 mL) was stirred at room temperature for 16 h under N₂ atmosphere. The resulting mixture was poured into H₂O (20 mL) and then extracted with EtOAc (20 mL×3). The combined organic layers were washed with brine (30 mL), dried over Na₂SO₄, and then concentrated in vacuo. The residue was purified by Prep-TLC, eluting with 3% MeOH in DCM to give (2S,3R)-3-(3-chlorophenyl)-2-methylbutanamide (70 mg, yield: 85%) as yellow oil. ESI-MS [M+H]+: 212.1.

Synthesis of (2S,3R)-3-(3-chlorophenyl)-N-(6-(((6-cyclopropylimidazo[1,2-a]pyridin-2-yl)methyl)amino)pyrimidin-4-yl)-2-methylbutanamide. A mixture of (2S,3R)-3-(3-chlorophenyl)-2-methylbutanamide (30 mg, 0.14 mmol), 6-bromo-N-((6-cyclopropylimidazo[1,2-a]pyridin-2-yl)methyl)pyrimidin-4-amine (31 mg, 0.09 mmol), Xantphos (10 mg, 0.018 mmol), Cs₂CO₃ (88 mg, 0.27 mmol) and Pd₂(dba)₃ (8 mg, 0.009 mmol) in 1,4-dioxane (3.0 mL) was stirred at 95° C. for 16 h under N₂ atmosphere. The mixture was cooled to room temperature and diluted with H₂O (15 mL) and then extracted with EtOAc (20 mL×3). The combined organic layers were washed with brine (10 mL), dried over Na₂SO₄, and concentrated in vacuo. The residue was purified by Prep-TLC, eluting with 5% MeOH in DCM to give the (2S,3R)-3-(3-chlorophenyl)-N-(6-(((6-cyclopropylimidazo[1,2-a]pyridin-2-yl)methyl)amino)pyrimidin-4-yl)-2-methylbutanamide (20 mg, yield: 47%) as a white solid. ESI-MS [M+H]⁺: 475.2. 1H NMR (400 MHz, DMSO) δ10.24 (s, 1H), 8.27 (s, 1H), 8.14 (s, 1H), 7.78 (s, 1H), 7.58 (s, 1H), 7.36-7.34 (m, 2H), 7.30-7.14 (m, 4H), 6.94 (d, J=9.3 Hz, 1H), 4.53 (s, 2H), 3.07-3.00 (m, 1H), 2.95-2.88 (m, 1H), 1.94-1.87 (m, 1H), 1.16 (d, J=6.9 Hz, 3H), 1.06 (d, J=6.6 Hz, 3H), 0.91-0.88 (m, 2H), 0.65-0.64 (m, 2H).

### Example 134

### Synthesis of (2R,3S)-3-(3-chlorophenyl)-N-(6-(((6-cyclopropylimidazo[1,2-a]pyridin-2-yl)methyl)amino)pyrimidin-4-yl)-2-methylbutanamide (I-134)

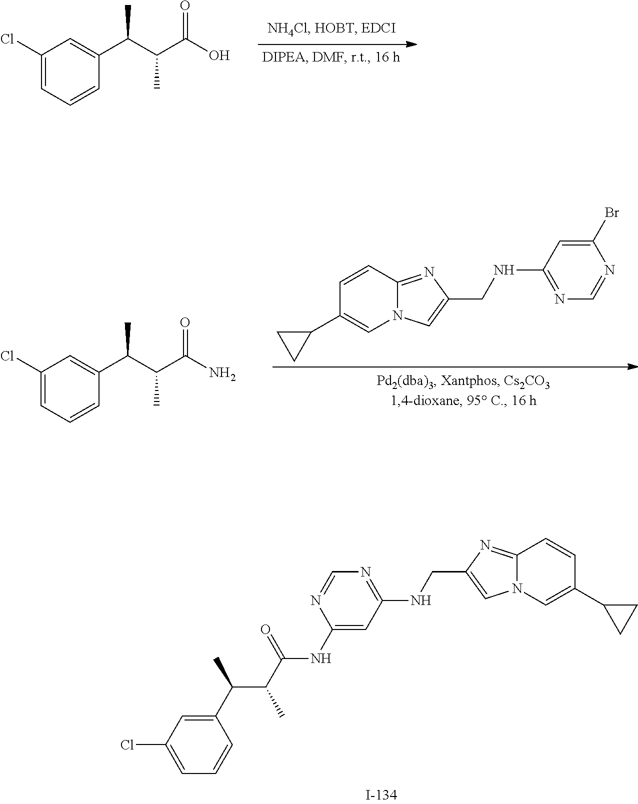 [SMILES: C[C@H](c1cccc(Cl)c1)[C@@H](C)C(=O)O ; C[C@H](c1cccc(Cl)c1)[C@@H](C)C(N)=O ; Brc1cc(NCc2cn3cc(C4CC4)ccc3n2)ncn1 ; C[C@H](c1cccc(Cl)c1)[C@@H](C)C(=O)Nc1cc(NCc2cn3cc(C4CC4)ccc3n2)ncn1]

Synthesis of (2R,3S)-3-(3-chlorophenyl)-2-methylbutanamide. A mixture of (2R,3S)-3-(3-chlorophenyl)-2-methylbutanoic acid (83 mg, 0.39 mmol), NH₄Cl (127 mg, 2.4 mmol), HOBT (108 mg, 0.80 mmol), DIPEA (310 mg, 2.4 mmol) and EDCI (153 mg, 0.80 mmol) in DMF (5.0 mL) was stirred at room temperature for 16 h under N₂ atmosphere. The resulting mixture was poured into H₂O (20 mL) and then extracted with EtOAc (20 mL×3). The combined organic layers were washed with brine (30 mL), dried over Na₂SO₄, and then concentrated in vacuo. The residue was purified by Prep-TLC, eluting with 3% MeOH in DCM to give (2R,3S)-3-(3-chlorophenyl)-2-methylbutanamide (70 mg, yield: 85%) as a yellow oil. ESI-MS [M+H]+: 212.1.

Synthesis of (2R,3S)-3-(3-chlorophenyl)-N-(6-(((6-cyclopropylimidazo[1,2-a]pyridin-2-yl)methyl)amino)pyrimidin-4-yl)-2-methylbutanamide. A mixture of (2R,3S)-3-(3-chlorophenyl)-2-methylbutanamide (70 mg, 0.33 mmol), 6-bromo-N-((6-cyclopropylimidazo[1,2-a]pyridin-2-yl)methyl)pyrimidin-4-amine (75 mg, 0.22 mmol), Xantphos (23 mg, 0.040 mmol), Cs₂CO₃ (176 mg, 0.54 mmol) and Pd₂(dba)₃ (18 mg, 0.020 mmol) in 1,4-dioxane (6.0 mL) was stirred at 95° C. for 16 h under N₂ atmosphere. The mixture was cooled to room temperature and concentrated in vacuo. The residue was diluted with H₂O (20 mL) and then extracted with EtOAc (20 mL×3). The combined organic layers were washed with brine (30 mL), dried over Na₂SO₄, and concentrated in vacuo. The residue was purified by Prep-TLC, eluting with 5% MeOH in DCM to give the (2R,3S)-3-(3-chlorophenyl)-N-(6-(((6-cyclopropylimidazo[1,2-a]pyridin-2-yl)methyl)amino)pyrimidin-4-yl)-2-methylbutanamide (21.8 mg, yield: 21%) as a white solid. ESI-MS [M+H]⁺: 475.1. ¹H NMR (400 MHz, DMSO) δ10.25 (s, 1H), 8.27 (s, 1H), 8.14 (s, 1H), 7.79 (s, 1H), 7.60 (d, J=18.1 Hz, 1H), 7.38-7.33 (m, 2H), 7.28-7.24 (m, 1H), 7.21-7.16 (m, 3H), 6.94 (d, J=9.4 Hz, 1H), 4.53 (s, 2H), 3.05-2.99 (m, 1H), 2.94-2.88 (m, 1H), 1.93-1.86 (m, 1H), 1.16 (d, J=6.9 Hz, 3H), 1.06 (d, J=6.6 Hz, 3H), 0.92-0.88 (m, 2H), 0.67-0.63 (m, 2H).

### Example 135

### Synthesis of (3S,5R)-1-(6-(((6-amino-2,4-dimethylpyridin-3-yl)methyl)amino)pyrimidin-4-yl)-5-(6-cyclopropylimidazo[1,2-a]pyridin-2-yl)pyrrolidin-3-ol (I-135)

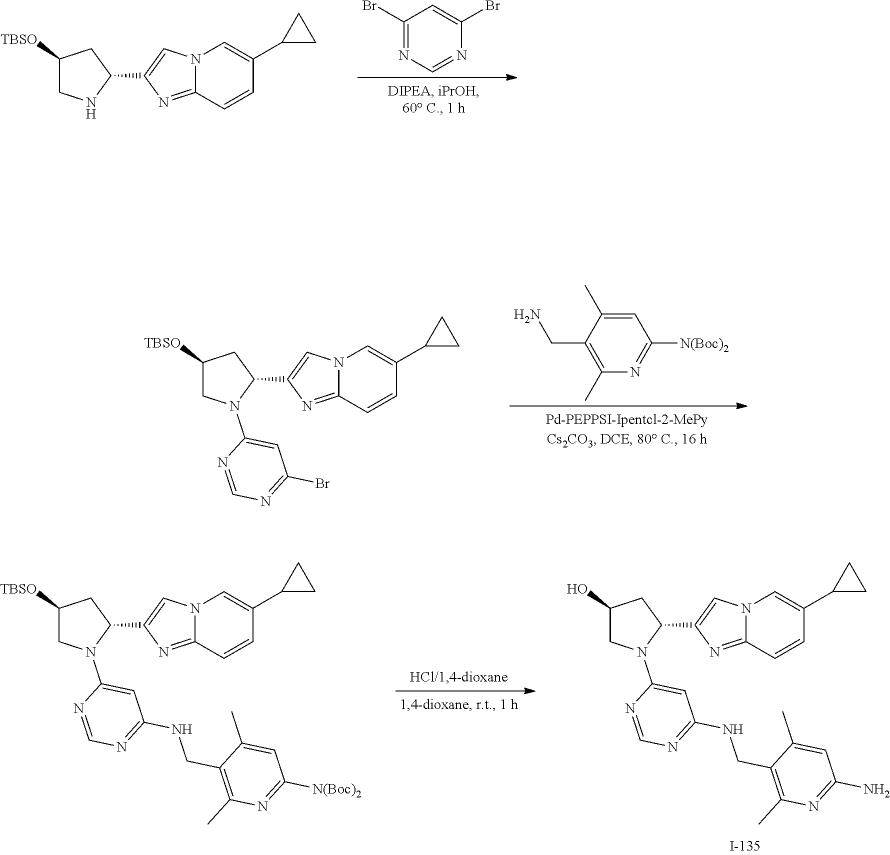

Synthesis of 2-((2R,4S)-1-(6-bromopyrimidin-4-yl)-4-((tert-butyldimethylsilyl)oxy)pyrrolidin-2-yl)-6-cyclopropylimidazo[1,2-a]pyridine. A mixture of 2-((2R,4S)-4-((tert-butyldimethylsilyl)oxy)pyrrolidin-2-yl)-6-cyclopropylimid azo[1,2-a]pyridine (455 mg, 1.27 mmol), 4,6-dibromopyrimidine (333 mg, 1.40 mmol) and DIPEA (492 mg, 3.81 mmol) in iPrOH (12 mL) was stirred at 60° C. for 1 h. The mixture was cooled to room temperature and concentrated in vacuo. The residue was purified by column chromatography on silica gel, eluting with 0˜60% EtOAc in PE to give 2-((2R,4S)-1-(6-bromopyrimidin-4-yl)-4-((tert-butyldimethylsilyl)oxy)pyrrolidin-2-yl)-6-cyclopropylimidazo[1,2-a]pyridine (400 mg, yield: 61%) as yellow solid. ESI-MS [M+H]⁺:514.1.

Synthesis of tert-butyl (tert-butoxycarbonyl)(5-(((6-((2R,4S)-4-((tert-butyldimethylsilyl)oxy)-2-(6-cyclopropylimidazo[1,2-a]pyridin-2-yl)pyrrolidin-1-yl)pyrimidin-4-yl)amino)methyl)-4,6-dimethylpyridin-2-yl)carbamate. A mixture of 2-((2R,4S)-1-(6-bromopyrimidin-4-yl)-4-((tert-butyldimethylsilyl)oxy)pyrrolidin-2-yl)-6-cyclopropylimidazo[1,2-a]pyridine (150 mg, 0.29 mmol), tert-butyl (5-(aminomethyl)-4,6-dimethylpyridin-2-yl)(tert-butoxycarbonyl)carbamate (255 mg, 0.73 mmol), Cs₂CO₃ (284 mg, 0.87 mmol) and Pd-PEPPSI-IPENT^{cl}-2-MePy (49 mg, 0.058 mmol) in DCE (5.0 mL) was stirred at 80° C. for 16 h under N₂ atmosphere. The mixture was cooled to room temperature. The resulting mixture was filtered through Celite® and the filter cake was washed with DCM/MeOH (V/V=10/1, 20 mL). The filtrate was concentrated in vacuo. The residue was purified by Prep-TLC, eluting with 10% MeOH in DCM to give tert-butyl (tert-butoxycarbonyl)(5-(((6-((2R,4S)-4-((tert-butyldimethylsilyl)oxy)-2-(6-cyclopropylimidazo[1,2-a]pyridin-2-yl)pyrrolidin-1-yl)pyrimidin-4-yl)amino)methyl)-4,6-dimethylpyridin-2-yl)carbamate (100 mg, yield: 43%) as a pale solid. ESI-MS [M+H]⁺: 785.4.

Synthesis of (3S,5R)-1-(6-(((6-amino-2,4-dimethylpyridin-3-yl)methyl)amino)pyrimidin-4-yl)-5-(6-cyclopropylimidazo[1,2-a]pyridin-2-yl)pyrrolidin-3-ol. To a solution of tert-butyl (tert-butoxycarbonyl)(5-(((6-((2R,4S)-4-((tert-butyldimethylsilyl)oxy)-2-(6-cyclopropylimidazo[1,2-a]pyridin-2-yl)pyrrolidin-1-yl)pyrimidin-4-yl)amino)methyl)-4,6-dimethylpyridin-2-yl)carbamate (150 mg, 0.13 mmol) in 1,4-dioxane (10 mL) was added HCl (4 N in 1,4-dioxane, 3 mL) at room temperature. The mixture was stirred at room temperature for 1 h and then concentrated in vacuo. The residue was neutralized with NH₃ (7M in MeOH), concentrated and purified by Prep-TLC, eluting with 10% MeOH in DCM to give (3S,5R)-1-(6-(((6-amino-2,4-dimethylpyridin-3-yl)methyl)amino)pyrimidin-4-yl)-5-(6-cyclopropylimidazo[1,2-a]pyridin-2-yl)pyrrolidin-3-ol (36 mg, yield: 59%) as a pale solid. ESI-MS [M+H]+: 471.2. 1H NMR (400 MHz, DMSO) δ8.22 (s, 1H), 7.97 (s, 1H), 7.51 (s, 1H), 7.35 (d, J=9.3 Hz, 1H), 6.94 (dd, J=9.3, 1.6 Hz, 1H), 6.43 (s, 1H), 6.07 (s, 1H), 5.61 (s, 2H), 5.36 (s, 1H), 5.05 (s, 2H), 4.45 (s, 1H), 4.20 (d, J=29.5 Hz, 2H), 3.69 (s, 1H), 2.35-2.16 (m, 5H), 2.07 (s, 3H), 1.92-1.86 (m, 1H), 0.91-0.87 (m, 2H), 0.65-0.62 (m, 2H).

### Example 136

### Synthesis of N⁴-((6-cyclopropylimidazo[1,2-a]pyridin-2-yl)methyl)-N⁶-(2,4-dimethoxybenz yl)pyrimidine-4,6-diamine (I-136)

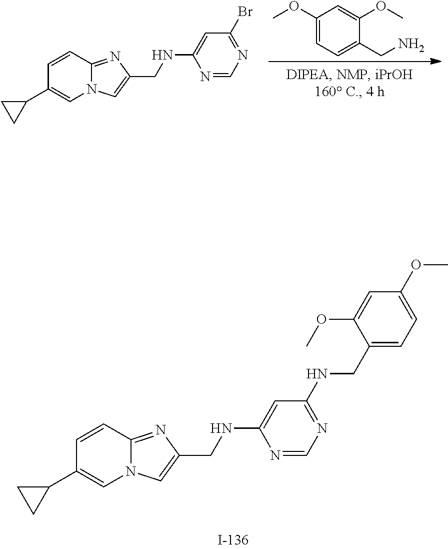

Synthesis of N⁴-((6-cyclopropylimidazo[1,2-a]pyridin-2-yl)methyl)-N⁶-(2,4-dimethoxybenzyl)pyrimidine-4,6-diamine. A mixture of 6-chloro-N-((6-cyclopropylimidazo[1,2-a]pyridin-2-yl)methyl)pyrimidin-4-amine (1.4 g, 4.7 mmol), (2,4-dimethoxyphenyl)methanamine (1.17 g, 7.0 mmol) and DIPEA (1.8 g, 14.0 mmol) in i-PrOH (5.0 mL) and NMP (5.0 mL) was stirred in a sealed tube. After degassing with N₂ for 1 min, the reaction mixture was irradiated in microwave at 160° C. for 8 h. The resulting mixture was cooled to room temperature and concentrated in vacuo. The residue was purified by column chromatography on silica gel, eluting with 0˜10% MeOH/DCM to give N⁴-((6-cyclopropylimidazo[1,2-a]pyridin-2-yl)methyl)-N⁶-(2,4-dimethoxybenzyl)pyrimidine-4,6-diamine give (25 mg, yield: 12%) as a pale solid. ESI-MS [M+H]+: 431.2. 1H NMR (400 MHz, DMSO) δ8.29 (s, 1H), 7.91 (s, 1H), 7.55 (s, 1H), 7.36 (d, J=8.0 Hz, 1H), 7.04-6.94 (m, 3H), 6.83 (t, J=6.0 Hz, 1H), 6.52 (d, 1H), 6.41 (d, J=8.0 Hz, 1H), 5.43 (s, 1H), 4.43 (d, J=8.0 Hz, 2H), 4.23 (s, 2H), 3.78 (s, 3H), 3.72 (s, 3H), 1.94-1.87 (m, 1H), 0.94-0.89 (m, 2H), 0.68-0.64 (m, 2H).

### Example 137

### Synthesis of (3S,5R)-1-(6-(((3-chloro-1H-pyrrolo[2,3-b]pyridin-5-yl)methyl)amino)pyrimidin-4-yl)-5-(6-cyclopropylimidazo[1,2-a]pyridin-2-yl)pyrrolidin-3-ol (I-137)

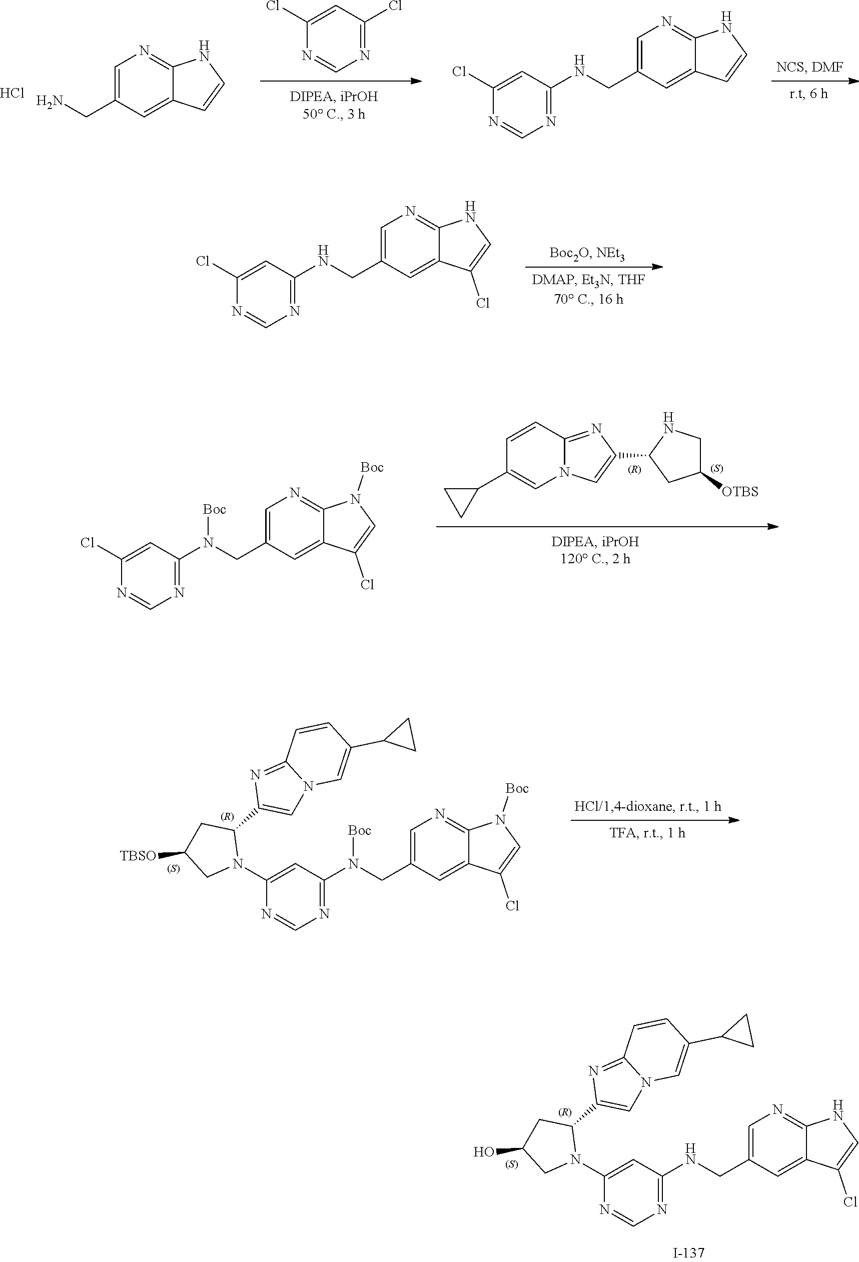

Synthesis of N-((1H-pyrrolo[2,3-b]pyridin-5-yl)methyl)-6-chloropyrimidin-4-amine. A mixture of (1H-pyrrolo[2,3-b]pyridin-5-yl)methanamine hydrochloride (257 mg, 1.4 mmol), DIPEA (426 mg, 3.3 mmol) and 4,6-dichloropyrimidine (253 mg, 1.7 mol) in i-PrOH (6.0 mL) was stirred at 50° C. for 3 h. The mixture was cooled to room temperature and concentrated in vacuo. The residue was purified by column chromatography on silica gel, eluting with 0˜5% MeOH in DCM to give N-((1H-pyrrolo[2,3-b]pyridin-5-yl)methyl)-6-chloropyrimidin-4-amine (160 mg, yield: 44%) as a yellow solid. ESI-MS [M+H]⁺: 260.1.

Synthesis of 6-chloro-N-((3-chloro-1H-pyrrolo[2,3-b]pyridin-5-yl)methyl)pyrimidin-4-amine. To a solution of N-((1H-pyrrolo[2,3-b]pyridin-5-yl)methyl)-6-chloropyrimidin-4-amine (80 mg, 0.31 mmol) in DMF (6.0 mL) was added NCS (83 mg, 0.62 mol) at room temperature. The mixture was stirred at room temperature for 6 h under N₂ at atmosphere. The reaction was quenched with H₂O (20 mL) and then extracted with EtOAc (20 mL×3). The combined organic layers were washed with brine (30 mL), dried over Na₂SO₄, and concentrated in vacuo. The residue was purified by Prep-TLC, eluting with 5% MeOH in DCM to give 6-chloro-N-((3-chloro-1H-pyrrolo[2,3-b]pyridin-5-yl)methyl)pyrimidin-4-amine (88 mg, yield: 97%) as a yellow solid. ESI-MS [M+H]⁺: 294.1.

Synthesis of tert-butyl 5-(((tert-butoxycarbonyl)(6-chloropyrimidin-4-yl)amino)methyl)-3-chloro-1H-pyrrolo[2,3-b]pyridine-1-carboxylate. To a solution of 6-chloro-N-((3-chloro-1H-pyrrolo[2,3-b]pyridin-5-yl)methyl)pyrimidin-4-amine (88 mg, 0.30 mol) in THF (10 mL) was added DMAP (18 mg, 0.15 mol), Boc₂O (392 mg, 1.8 mmol) and Et₃N (243 mg, 2.4 mmol). The mixture was stirred at 70° C. for 16 h then cooled to room temperature. H₂O (20 mL) was added and extracted with EtOAc (20 mL×3). The combined organic layers were washed with brine (30 mL), dried over Na₂SO₄, and concentrated in vacuo. The residue was purified by column chromatography on silica gel, eluting with 0˜5% MeOH in DCM to give tert-butyl 5-(((tert-butoxycarbonyl)(6-chloropyrimidin-4-yl)amino)methyl)-3-chloro-1H-pyrrolo[2,3-b]pyridine-1-carboxylate (145 mg, yield: 97%) as a yellow solid. ESI-MS [M+H]+: 494.1.

Synthesis of tert-butyl 5-(((tert-butoxycarbonyl)(6-((2R,4S)-4-((tert-butyldimethylsilyl)oxy)-2-(6-cyclopropylimidazo[1,2-a]pyridin-2-yl)pyrrolidin-1-yl)pyrimidin-4-yl)amino)methyl)-3-chloro-1H-pyrrolo[2,3-b]pyridine-1-carboxylate. A mixture of tert-butyl 5-(((tert-butoxycarbonyl)(6-chloropyrimidin-4-yl)amino)methyl)-3-chloro-1H-pyrrolo[2,3-b]pyridine-1-carboxylate (145 mg, 0.29 mmol), DIPEA (112 mg, 0.87 mmol) and 2-((2R,4S)-4-((tert-butyldimethylsilyl)oxy)pyrrolidin-2-yl)-6-cyclopropylimidazo[1,2-a]pyridine (125 mg, 0.35 mmol) in i-PrOH (2.0 mL) was stirred in a sealed tube. After degassing with N₂ for 1 min. The reaction mixture was irradiated in microwave at 120° C. for 2 h. The mixture was cooled to room temperature and concentrated in vacuo. The residue was purified by Prep-TLC, eluting with 5% MeOH in DCM to give tert-butyl 5-(((tert-butoxycarbonyl)(6-((2R,4S)-4-((tert-butyldimethylsilyl)oxy)-2-(6-cyclopropylimidazo[1,2-a]pyridin-2-yl)pyrrolidin-1-yl)pyrimidin-4-yl)amino)methyl)-3-chloro-1H-pyrrolo[2,3-b]pyridine-1-carboxylate (100 mg, yield: 48%) as a yellow solid. ESI-MS [M+H]+: 715.3.

Synthesis of 2-((6-cyclopropylimidazo[1,2-a]pyridin-2-yl)methyl)thiazole-5-carboxylic acid. To a solution of tert-butyl 5-(((tert-butoxycarbonyl)(6-((2R,4S)-4-((tert-butyldimethylsilyl)oxy)-2-(6-cyclopropylimidazo[1,2-a]pyridin-2-yl)pyrrolidin-1-yl)pyrimidin-4-yl)amino)methyl)-3-chloro-1H-pyrrolo[2,3-b]pyridine-1-carboxylate (90 mg, 0.11 mol) in 1,4-ioxane (3 mL) was added HCl (4 N in 1,4-dioxane, 2.0 mL). After stirring at room temperature for 1 h, the mixture was concentrated to remove volatile and the residue was re-dissolved in DCM (5.0 mL). TFA (1.0 mL) was added to the above mixture. The resulting mixture was stirred at room temperature for 1 h. The mixture was then neutralized with NaHCO₃ (sat. aq., 10 mL) and extracted with DCM (20 mL×3). The combined organics were washed with brine (30 mL), dried over Na₂SO₄, and concentrated in vacuo. The residue was purified by Prep-TLC, eluting with 10% MeOH in DCM to give (3S,5R)-1-(6-(((3-chloro-1H-pyrrolo[2,3-b]pyridin-5-yl)methyl)amino)pyrimidin-4-yl)-5-(6-cyclopropylimidazo[1,2-a]pyridin-2-yl)pyrrolidin-3-ol (34.8 mg, yield: 63%) as a white solid. ESI-MS [M+H]⁺: 501.1. ¹H NMR (400 MHz, MeOD) δ8.12 (s, 1H), 7.98 (s, 1H), 7.91 (s, 1H), 7.71 (s, 1H), 7.40-7.33 (m, 2H), 7.23 (d, J=9.3 Hz, 1H), 6.94 (d, J=9.4 Hz, 1H), 5.34 (s, 1H), 5.07 (s, 1H), 4.51-4.47 (m, 3H), 3.92-3.88 (m, 1H), 3.72 (s, 1H), 2.45-2.41 (m, 1H), 2.29-2.25 (m, 1H), 1.90-1.84 (m, 1H), 0.98-0.94 (m, 2H), 0.68-0.64 (m, 2H).

### Example 138

### Synthesis of N⁴-(((1S,2S)-2-(3-chlorophenyl)cyclopropyl)methyl)-N-((6-cyclopropylimidazo[1,2-a]pyridin-2-yl)methyl)pyrimidine-4,6-diamine (I-138)

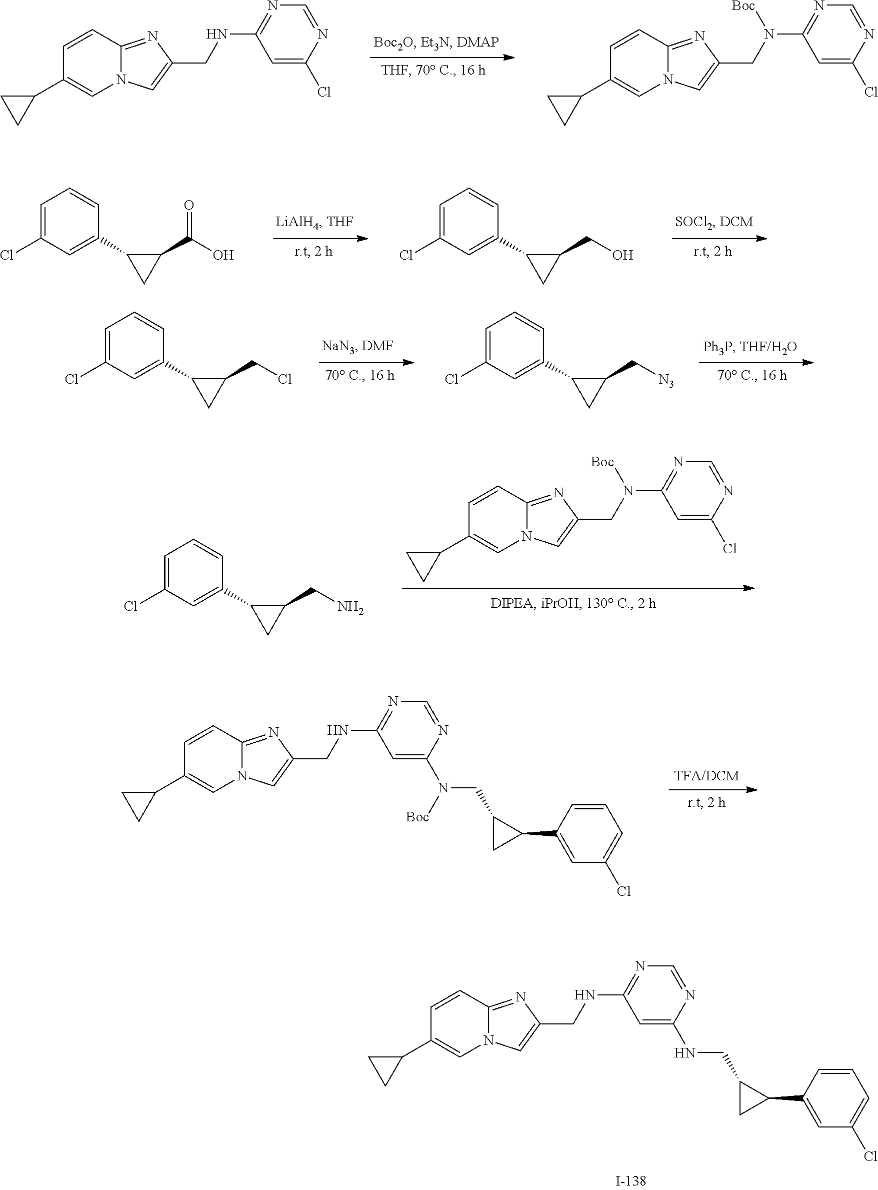

Synthesis of tert-butyl (6-chloropyrimidin-4-yl)((6-cyclopropylimidazo[1,2-a]pyridin-2-yl)methyl)carbamate. To a solution of 6-chloro-N-((6-cyclopropylimidazo[1,2-a]pyridin-2-yl)methyl)pyrimidin-4-amine (300 mg, 1.0 mol) in THF (20 mL) was added Boc₂O (436 mg, 2.0 mmol), DMAP (61 mg, 0.50 mol) and Et₃N (305 mg, 3.0 mmol). The reaction mixture was stirred at 70° C. for 16 h. The mixture was cooled to room temperature and concentrated in vacuo. The residue was purified by Prep-TLC, eluting with 2% MeOH in DCM to give tert-butyl (6-chloropyrimidin-4-yl)((6-cyclopropylimidazo[1,2-a]pyridin-2-yl)methyl)carbamate (320 mg, yield: 80%) as yellow solid. ESI-MS [M+H]⁺: 400.2.

Synthesis of ((1S,2S)-2-(3-chlorophenyl)cyclopropyl)methanol. To a solution of (1S,2S)-2-(3-chlorophenyl)cyclopropane-1-carboxylic acid (300 mg, 1.53 mmol) in THF (15 mL) was added slowly LiAlH₄ (1 N in THF, 1.8 mL) at 0° C. The mixture was stirred at room temperature for 2 h under N₂ atmosphere. The reaction mixture was quenched with NaSO₄·10H₂O. The resulting mixture was stirred for another 0.5 h, then filtered and the filter cake was washed with EtOAc (30 mL). The filtrate was concentrated in vacuo to give ((1S,2S)-2-(3-chlorophenyl)cyclopropyl)methanol as a colorless oil (300 mg, crude), which was used directly in the next step without further purification. ESI-MS [M+H]+: 183.1.

Synthesis of 1-chloro-3-((1S,2S)-2-(chloromethyl)cyclopropyl)benzene. To a solution of ((1S,2S)-2-(3-chlorophenyl)cyclopropyl)methano (300 mg, crude) in DCM (10 mL) was added SOCl₂ (1.0 mL) at 0° C. The mixture was stirred at room temperature for 2 h under N₂ atmosphere. The resulting mixture was concentrated in vacuo to give 1-chloro-3-((1S,2S)-2-(chloromethyl)cyclopropyl)benzene as a colorless oil (310 mg, crude), which was used directly in the next step without further purification. ESI-MS [M+H]⁺: 201.1.

Synthesis of 1-((1S,2S)-2-(azidomethyl)cyclopropyl)-3-chlorobenzene. To a solution of 1-chloro-3-((1S,2S)-2-(chloromethyl)cyclopropyl)benzene (310 mg, crude) in DMF (10 mL) was added NaN₃ (145 mg, 2.23 mmol). The mixture was stirred at 70° C. for 16 h under N₂ atmosphere. The mixture was cooled to room temperature, diluted with H₂O (20 mL) and then extracted with EtOAc (20 mL×3). The combined organics were washed with brine (30 mL), dried over Na₂SO₄, and concentrated in vacuo to give 1-((1S,2S)-2-(azidomethyl)cyclopropyl)-3-chlorobenzene (320 mg, crude) as a white solid, which was used directly in the next step without further purification. ESI-MS [M+H]⁺: 208.1.

Synthesis of ((1S,2S)-2-(3-chlorophenyl)cyclopropyl)methanamine. A mixture of PPh₃ (1.14 g, 4.34 mmol) and 1-((1S,2S)-2-(azidomethyl)cyclopropyl)-3-chlorobenzene (320 mg, crude) in THF/H₂O (20 mL/3.0 mL) was stirred at 70° C. for 16 h under N₂ atmosphere. The reaction mixture was cooled to room temperature, diluted with H₂O (20 mL), and then extracted with EtOAc (30 mL×3). The combined organics were washed with brine (30 mL), dried over Na₂SO₄, and concentrated in vacuo. The residue was purified by column chromatography on silica gel, eluting with 0˜5% MeOH in DCM to give ((1S,2S)-2-(3-chlorophenyl)cyclopropyl)methanamine (200 mg, 72% over 4 steps) as a white solid. ESI-MS [M+H]⁺: 182.1.

Synthesis of tert-butyl (((1S,2S)-2-(3-chlorophenyl)cyclopropyl)methyl)(6-(((6-cyclopropylimidazo[1,2-a]pyridin-2-yl)methyl)amino)pyrimidin-4-yl)carbamate. A mixture of ((1S,2S)-2-(3-chlorophenyl)cyclopropyl)methanamine (120 mg, 0.66 mmol), tert-butyl (6-chloropyrimidin-4-yl)((6-cyclopropylimidazo[1,2-a]pyridin-2-yl)methyl)carbamate (289 mg, 0.72 mmol) and DIPEA (426 mg, 3.3 mmol) in i-PrOH (15 mL) was stirred in a sealed tube. After degassing with N₂ for 1 min, the mixture was irradiated in microwave at 130° C. for 2 h. The resulting mixture was cooled to room temperature, diluted with H₂O (20 mL), and then extracted with EtOAc (20 mL×3). The combined organics were washed with brine (20 mL), dried over Na₂SO₄, and concentrated in vacuo. The residue was purified by Prep-TLC, eluting with 5% MeOH in DCM to give tert-butyl (((1S,2S)-2-(3-chlorophenyl)cyclopropyl)methyl)(6-(((6-cyclopropylimidazo[1,2-a]pyridin-2-yl)methyl)amino)pyrimidin-4-yl)carbamate (60 mg, yield: 17%). ESI-MS [M+H]⁺: 545.1.

Synthesis of N⁴-(((1S,2S)-2-(3-chlorophenyl)cyclopropyl)methyl)-N⁶-((6-cyclopropylimidazo[1,2-a]pyridin-2-yl)methyl)pyrimidine-4,6-diamine. To a solution of tert-butyl (((1S,2S)-2-(3-chlorophenyl)cyclopropyl)methyl)(6-(((6-cyclopropylimidazo[1,2-a]pyridin-2-yl)methyl)amino)pyrimidin-4-yl)carbamate (60 mg, 0.11 mmol) in DCM (10 mL) was added TFA (1.0 mL). After stirring at room temperature for 2 h, the mixture was concentrated in vacuo to remove volatile. The residue was diluted with NH₃ (7 N in MeOH, 1 mL) and then concentrated in vacuo. The crude product was purified by Prep-TLC, eluting with 10% MeOH in DCM to give N⁴-(((1S,2S)-2-(3-chlorophenyl)cyclopropyl)methyl)-N⁶-((6-cyclopropylimidazo [1,2-a]pyridin-2-yl)methyl)pyrimidine-4,6-diamine (30 mg, yield: 61%) as a pale solid. ESI-MS [M+H]+: 445.2. ¹H NMR (400 MHz, DMSO) δ8.29 (s, 1H), 7.92 (s, 1H), 7.58 (s, 1H), 7.35 (d, J=9.3 Hz, 1H), 7.23 (t, J=7.8 Hz, 1H), 7.17-7.12 (m, 1H), 7.09 (s, 1H), 7.01-6.89 (m, 3H), 6.79-6.71 (s, 1H), 5.45 (s, 1H), 4.43 (d, J=5.3 Hz, 2H), 3.22 (s, 1H), 3.18-3.06 (m, 1H), 1.98-1.73 (m, 2H), 1.37-1.18 (m, 1H), 0.98-0.77 (m, 4H), 0.65-0.63 (m, 3H).

### Example 139

### Synthesis of (3S,5R)-1-(6-(((3-chloro-6-fluoro-1H-indol-5-yl)methyl)amino)pyrimidin-4-yl)-5-(6-cyclopropylimidazo[1,2-a]pyridin-2-yl)pyrrolidin-3-ol (I-139)

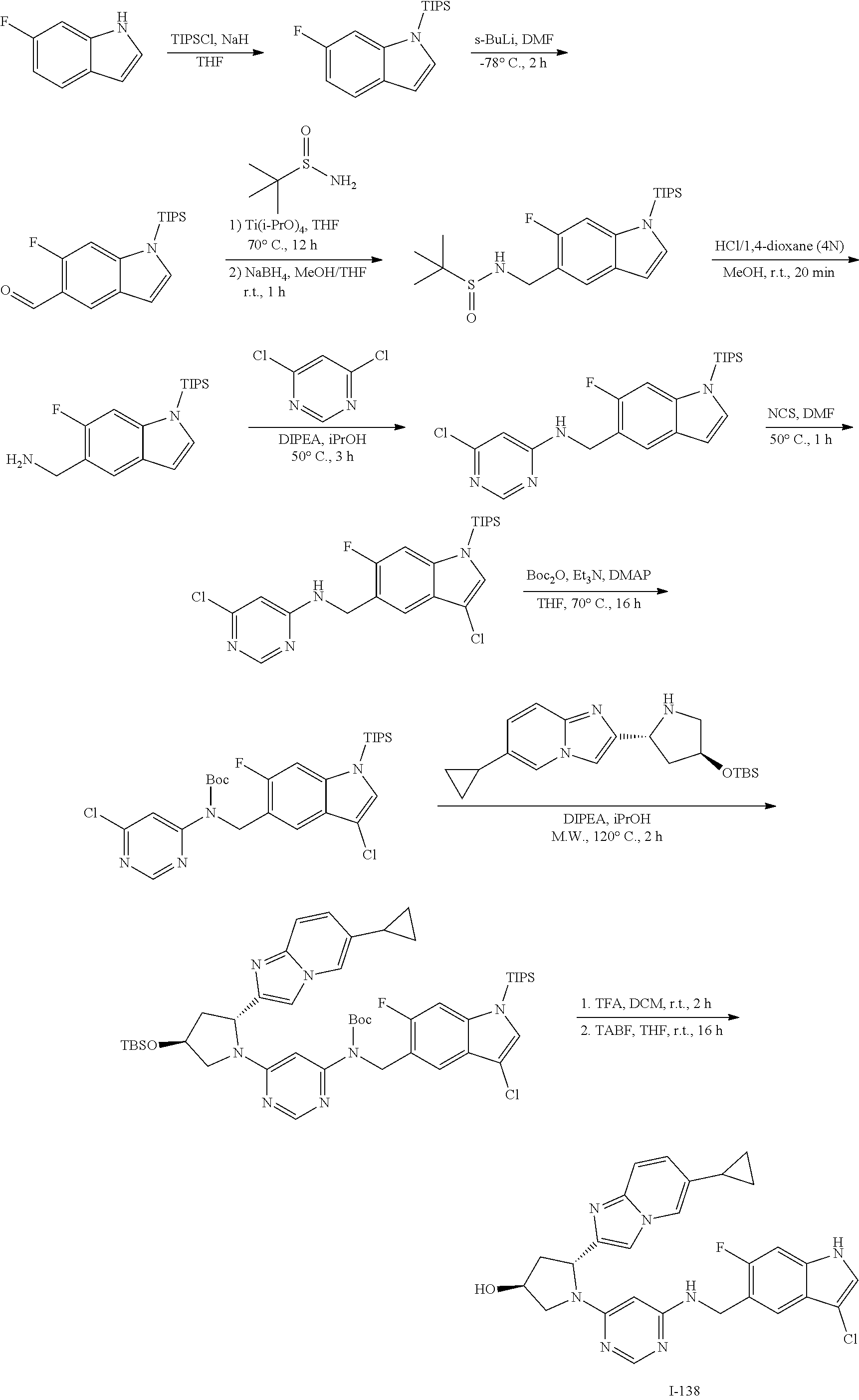

Synthesis of 6-fluoro-1-(triisopropylsilyl)-1H-indole. To a stirred solution of 6-fluoro-1H-indole (2.0 g, 14.8 mmol) in THF (50 mL) was added dropwise NaH (712 mg, 17.8 mmol, 60% dispersion in mineral oil) at 0° C. The mixture was warmed to room temperature and stirred at this temperature for 30 min. The resulting mixture was cooled to 0° C. and added TIPSCl (3.71 g, 19.2 mmol). The resulting mixture was warmed to room temperature and stirred until consumption of starting material as monitored by TLC. The reaction mixture was quenched NH₄Cl (sat. aq., 80 mL) at 0° C. and then extracted with EtOAc (3×60 mL). The combined organic layers were washed with brine (100 mL), dried over Na₂SO₄, and concentrated in vacuo. The residue was purified by column chromatography on silica gel, eluting with 0˜2% EtOAc in PE to give 6-fluoro-1-(triisopropylsilyl)-1H-indole (4.2 g, yield: 97%) as a colorless oil. ESI-MS [M+H]⁺: 292.2.

Synthesis of 6-fluoro-1-(triisopropylsilyl)-1H-indole-5-carbaldehyde. To a solution of 6-fluoro-1-(triisopropylsilyl)-1H-indole (4.0 g, 13.7 mmol) in THF (40 mL) was added dropwise sec-Butylithium (1.3 N in hexane, 16.4 mmol, 12.6 ml) at −78° C. After stirring at −78° C. for 2 h, DMF (3.0 g, 41.0 mmol) was added slowly. The resulting mixture was warmed to room temperature and stirred for another 2 h. The reaction mixture was quenched by NH₄C₁ (sat. aq., 60 mL) and then extracted with EtOAc (3×50 mL). The combined organic layers were washed with brine (80 mL), dried over Na₂SO₄, and concentrated in vacuo. The residue was purified by column chromatography on silica gel, eluting with 0˜10% EtOAc in PE to give 6-fluoro-1-(triisopropylsilyl)-1H-indole-5-carbaldehyde (3.5 g, yield: 80%) as a colorless oil. ESI-MS [M+H]⁺: 320.2.

Synthesis of N-((6-fluoro-1-(triisopropylsilyl)-1H-indol-5-yl)methyl)-2-methylpropane-2-sulfinamide. To a solution of 6-fluoro-1-(triisopropylsilyl)-1H-indole-5-carbaldehyde (3.7 g, 11.6 mmol) in THF (80 mL) was added 2-methylpropane-2-sulfinamide (2.8 g, 23.2 mol) and Ti(PrO)₄ (8.2 g, 13.1 mmol). After stirring at 70° C. for 12 h, to the mixture was added MeOH (15 mL) and NaBH₄ (1.3 g, 34.8 mmol) successively at 0° C. The resulting mixture was stirred at room temperature for another 1 h. The reaction mixture was quenched by NH₄Cl (sat. aq., 100 mL) and then extracted with EtOAc (3×80 mL). The combined organics were washed with brine (100 mL), dried over Na₂SO₄, and concentrated in vacuo. The residue was purified by column chromatography on silica gel, eluting with 0˜3% MeOH in DCM to give N-((6-fluoro-1-(triisopropylsilyl)-1H-indol-5-yl)methyl)-2-methylpropane-2-sulfinamide (3.5 g, yield: 71%) as a yellow oil. ESI-MS [M+H]⁺: 425.2.

Synthesis of (6-fluoro-]1-(triisopropylsilyl)-1H-indol-5-yl)methanamine. To a solution of N-((6-fluoro-1-(triisopropylsilyl)-1H-indol-5-yl)methyl)-2-methylpropane-2-sulfinamide (3.5 g, 8.24 mol) in MeOH (30 mL) was added HCl (4 N in 1,4-dioxane, 10 mL). After stirring at room temperature for 0.5 h, NaHCO₃ (sat. aq.) was added to adjust the pH value to 8˜9. The resulting mixture was extracted with EtOAc (3×50 mL). The combined organic layers were washed with brine (80 mL), dried over Na₂SO₄, and then concentrated in vacuo. The residue was purified by column chromatography on silica gel, eluting with 0˜5% MeOH in DCM to give (6-fluoro-]1-(triisopropylsilyl)-1H-indol-5-yl)methanamine (1.9 g, yield: 72%) as a yellow oil. ESI-MS [M+H]⁺: 321.2.

Synthesis of 6-chloro-N-((6-fluoro-1-(triisopropylsilyl)-1H-indol-5-yl)methyl)pyrimidin-4-amine. A mixture of (6-fluoro-]-1-(triisopropylsilyl)-1H-indol-5-yl)methanamine (1.9 g, 5.9 mmol), DIPEA (2.28 g, 17.7 mol) and 4,6-dichloropyrimidine (1.06 g, 7.1 mol) in i-PrOH (50 mL) was stirred at 50° C. for 3 h. The mixture was cooled to room temperature and concentrated in vacuo. The residue was purified by column chromatography on silica gel, eluting with 0˜3% MeOH in DCM to give 6-chloro-N-((6-fluoro-1-(triisopropylsilyl)-1H-indol-5-yl)methyl)pyrimidin-4-amine (1.5 g, yield: 58%) as a yellow solid. ESI-MS [M+H]⁺: 433.1.

Synthesis of 6-chloro-N-((3-chloro-6-fluoro-1-(triisopropylsilyl)-1H-indol-5-yl)methyl)pyrimidin-4-amine. To a solution of 6-chloro-N-((6-fluoro-1-(triisopropylsilyl)-1H-indol-5-yl)methyl)pyrimidin-4-amine (430 mg, 0.99 mmol) in DMF (10 mL) was added NCS (161 mg, 1.2 mol). After stirring at 50° C. for 1 h, the mixture was cooled to room temperature. The reaction mixture was quenched by H₂O (30 mL) and then extracted with EtOAc (30 mL×3). The combined organics were washed with brine (50 mL), dried over Na₂SO₄, and concentrated in vacuo. The residue was purified by column chromatography on silica gel, eluting with 0˜3% MeOH in DCM to give 6-chloro-N-((3-chloro-6-fluoro-1-(triisopropylsilyl)-1H-indol-5-yl)methyl)pyrimidin-4-amine (200 mg, yield: 43%) as a yellow solid. ESI-MS [M+H]+: 467.2.

Synthesis of tert-butyl ((3-chloro-6-fluoro-1-(triisopropylsilyl)-1H-indol-5-yl)methyl)(6-chloropyrimidin-4-yl)carbamate. To a solution of 6-chloro-N-((3-chloro-6-fluoro-1-(triisopropylsilyl)-1H-indol-5-yl)methyl)pyrimidin-4-amine (200 mg, 0.43 mol) in THF (10 mL) was added Boc₂O (281 mg, 1.29 mmol), DMAP (27 mg, 0.22 mol) and Et₃N (217 mg, 2.14 mmol). The mixture was stirred at 70° C. for 16 h then cooled to room temperature and concentrated in vacuo. The residue was purified by column chromatography on silica gel, eluting with 2% MeOH in DCM to give tert-butyl ((3-chloro-6-fluoro-1-(triisopropylsilyl)-1H-indol-5-yl)methyl)(6-chloropyrimidin-4-yl)carbamate (180 mg, yield: 74%) as yellow solid. ESI-MS [M+H]+: 567.2.

Synthesis of tert-butyl (6-((2R,4S)-4-((tert-butyldimethylsilyl)oxy)-2-(6-cyclopropylimidazo[1,2-a]pyridin-2-yl)pyrrolidin-1-yl)pyrimidin-4-yl)((3-chloro-6-fluoro-1-(triisopropylsilyl)-1H-indol-5-yl)methyl)carbamate. A mixture of tert-butyl ((3-chloro-6-fluoro-1-(triisopropylsilyl)-1H-indol-5-yl)methyl)(6-chloropyrimidin-4-yl)carbamate (90 mg, 0.16 mmol), DIPEA (62 mg, 0.48 mmol) and 2-((2R,4S)-4-((tert-butyldimethylsilyl)oxy)pyrrolidin-2-yl)-6-cyclopropylimidazo[1,2-a]pyridine (69 mg, 0.19 mol) in i-PrOH (2.0 mL) was stirred in sealed tube. After degassing with N₂ for 1 min, the mixture was irradiated in microwave at 120° C. for 2 h. The mixture was cooled to room temperature and concentrated in vacuo. The residue was purified by Prep-TLC, eluting with 3% MeOH in DCM to give tert-butyl (6-((2R,4S)-4-((tert-butyldimethylsilyl)oxy)-2-(6-cyclopropylimidazo[1,2-a]pyridin-2-yl)pyrrolidin-1-yl)pyrimidin-4-yl)((3-chloro-6-fluoro-1-(triisopropylsilyl)-1H-indol-5-yl)methyl)carbamate (80 mg, yield: 56%) as a yellow solid. ESI-MS [M+H]+: 888.5.

Synthesis of (3S,5R)-1-(6-(((3-chloro-6-fluoro-1H-indol-5-yl)methyl)amino)pyrimidin-4-yl)-5-(6-cyclopropylimidazo[1,2-a]pyridin-2-yl)pyrrolidin-3-ol. To a solution of tert-butyl (6-((2R,4S)-4-((tert-butyldimethylsilyl)oxy)-2-(6-cyclopropylimidazo[1,2-a]pyridin-2-yl)pyrrolidin-1-yl)pyrimidin-4-yl)((3-chloro-6-fluoro-1-(triisopropylsilyl)-1H-indol-5-yl)methyl)carbamate (60 mg, 0.068 mol) in DCM (5.0 mL) was added TFA (0.5 ml). After stirring at room temperature for 2 h, the mixture was concentrated in vacuo to remove volatile. The residue was diluted with NaHCO₃ (sat. aq., 10 mL) and then extracted with DCM (3×20 mL). The combined organic layers were concentrated in vacuo. The residue was re-dissolved in THF (5.0 mL) and then added TBAF (0.5 mL). After stirring at room temperature for 16 h, the mixture was quenched with H₂O (20 mL) and then extracted with DCM (3×20 mL). The combined organic layers were washed with brine (30 mL), dried over Na₂SO₄, and concentrated in vacuo. The residue was purified by Prep-TLC, eluting with 10% MeOH in DCM to give (3S,5R)-1-(6-(((3-chloro-6-fluoro-1H-indol-5-yl)methyl)amino)pyrimidin-4-yl)-5-(6-cyclopropylimidazo[1,2-a]pyridin-2-yl)pyrrolidin-3-ol (15 mg, yield: 43%) as a white solid. ESI-MS [M+H]+: 518.2. 1H NMR (400 MHz, MeOD) δ7.97 (s, 1H), 7.89 (s, 1H), 7.36 (s, 1H), 7.24-7.15 (m, 3H), 7.01-6.92 (m, 2H), 5.34 (s, 1H), 5.06 (s, 1H), 4.46 (d, J=34.9 Hz, 3H), 3.92-3.89 (m, 1H), 3.73 (s, 1H), 2.45-2.39 (m, 1H), 2.29-2.25 (m, 1H), 1.91-1.85 (m, 1H), 1.02-0.90 (m, 2H), 0.71-0.64 (m, 2H).

### Example 140

### Synthesis of (3S,5R)-5-(6-cyclopropylimidazo[1,2-a]pyridin-2-yl)-1-(6-(((6-fluoro-1H-indol-5-yl)methyl)amino)pyrimidin-4-yl)pyrrolidin-3-ol (I-140)

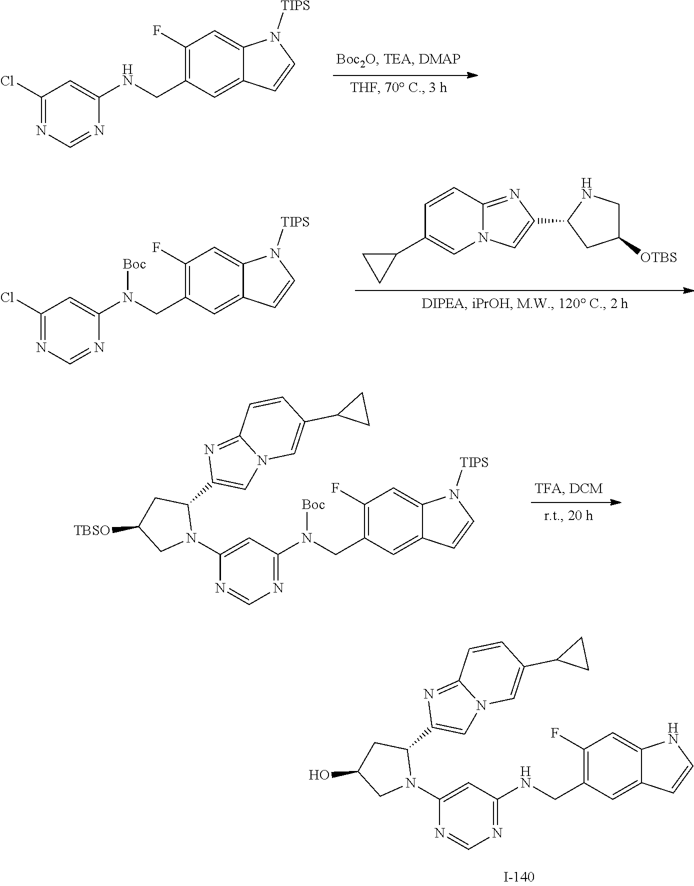

Synthesis of tert-butyl (6-chloropyrimidin-4-yl)((6-fluoro-1-(triisopropylsilyl)-1H-indol-5-yl)methyl)carbamate. To a solution of 6-chloro-N-((6-fluoro-1-(triisopropylsilyl)-1H-indol-5-yl)methyl)pyrimidin-4-amine (230 mg, 0.53 mol) in THF (10 mL) was added Boc₂O (347 mg, 1.59 mmol), DMAP (33 mg, 0.27 mol) and Et₃N (268 mg, 2.64 mmol). The mixture was stirred at 70° C. for 3 h. The mixture was cooled to room temperature and concentrated in vacuo. The residue was purified by Prep-TLC, eluting with 2% MeOH in DCM to give tert-butyl (6-chloropyrimidin-4-yl)((6-fluoro-1-(triisopropylsilyl)-1H-indol-5-yl)methyl)carbamate (200 mg, yield: 67%) as a yellow solid. ESI-MS [M+H]+: 567.2.

Synthesis of tert-butyl (6-((2R,4S)-4-((tert-butyldimethylsilyl)oxy)-2-(6-cyclopropylimidazo[1,2-a]pyridin-2-yl)pyrrolidin-1-yl)pyrimidin-4-yl)((6-fluoro-1-(triisopropylsilyl)-1H-indol-5-yl)methyl)carbamate. A mixture of tert-butyl (6-chloropyrimidin-4-yl)((6-fluoro-1-(triisopropylsilyl)-1H-indol-5-yl)methyl)carbamate (150 mg, 0.27 mmol), DIPEA (108 mg, 0.84 mmol) and 2-((2R,4S)-4-((tert-butyldimethylsilyl)oxy)pyrrolidin-2-yl)-6-cyclopropylimidazo[1,2-a]pyridine (122 mg, 0.34 mol) in i-PrOH (3 mL) was stirred in a sealed tube. The reaction mixture was irradiated in microwave at 120° C. for 2 h. The mixture was cooled to room temperature and concentrated in vacuo. The residue was purified by Prep-TLC, eluting with 3% MeOH in DCM to give tert-butyl (6-((2R,4S)-4-((tert-butyldimethylsilyl)oxy)-2-(6-cyclopropylimidazo[1,2-a]pyridin-2-yl)pyrrolidin-1-yl)pyrimidin-4-yl)((6-fluoro-1-(triisopropylsilyl)-1H-indol-5-yl)methyl)carbamate (80 mg, yield: 35%) as a yellow solid. ESI-MS [M+H]+: 855.5.

Synthesis of (3S,5R)-5-(6-cyclopropylimidazo[1,2-a]pyridin-2-yl)-1-(6-(((6-fluoro-1H-indol-5-yl)methyl)amino)pyrimidin-4-yl)pyrrolidin-3-ol. To a solution of tert-butyl (6-((2R,4S)-4-((tert-butyldimethylsilyl)oxy)-2-(6-cyclopropylimidazo[1,2-a]pyridin-2-yl)pyrrolidin-1-yl)pyrimidin-4-yl)((6-fluoro-1-(triisopropylsilyl)-1H-indol-5-yl)methyl)carbamate (60 mg, 0.07 mol) in DCM (5.0 mL) was added TFA (0.5 mL). After stirring at room temperature for 20 h, the mixture was concentrated, diluted with NaHCO₃ (sat. aq., 10 mL) and then extracted with DCM (3×20 mL). The combined organic layers were washed with brine (30 mL), dried over Na₂SO₄, and concentrated in vacuo. The residue was purified by Prep-TLC, eluting with 10% MeOH in DCM to give (3S,5R)-5-(6-cyclopropylimidazo[1,2-a]pyridin-2-yl)-1-(6-(((6-fluoro-1H-indol-5-yl)methyl)amino)pyrimidin-4-yl)pyrrolidin-3-ol (4.2 mg, yield: 12%) as a white solid. ESI-MS [M+H]⁺: 484.2. 1H NMR (400 MHz, MeOD) δ7.95 (s, 1H), 7.90 (s, 1H), 7.36 (s, 1H), 7.31-7.25 (m, 2H), 7.14 (d, J=3.1 Hz, 1H), 7.00-6.95 (m, 2H), 6.26 (s, 1H), 5.37 (s, 1H), 5.09 (s, 1H), 4.53-4.48 (m, 1H), 4.43-4.31 (m, 2H), 3.92-3.88 (m, 1H), 3.71 (s, 1H), 2.41-2.38 (m, 1H), 2.33-2.28 (m, 1H), 1.92-1.86 (m, 1H), 0.99-0.93 (m, 2H), 0.71-0.66 (m, 2H).

### Example 141

### Synthesis of 2-(3-chloroimidazo[1,5-b]pyridazin-5-yl)-N-(6-(((6-cyclopropyl-8-(3-methyl-2,4-dioxoimidazolidin-1-yl)imidazo[1,2-a]pyridin-2-yl)methyl)amino)pyrimidin-4-yl)acetamide (I-141)

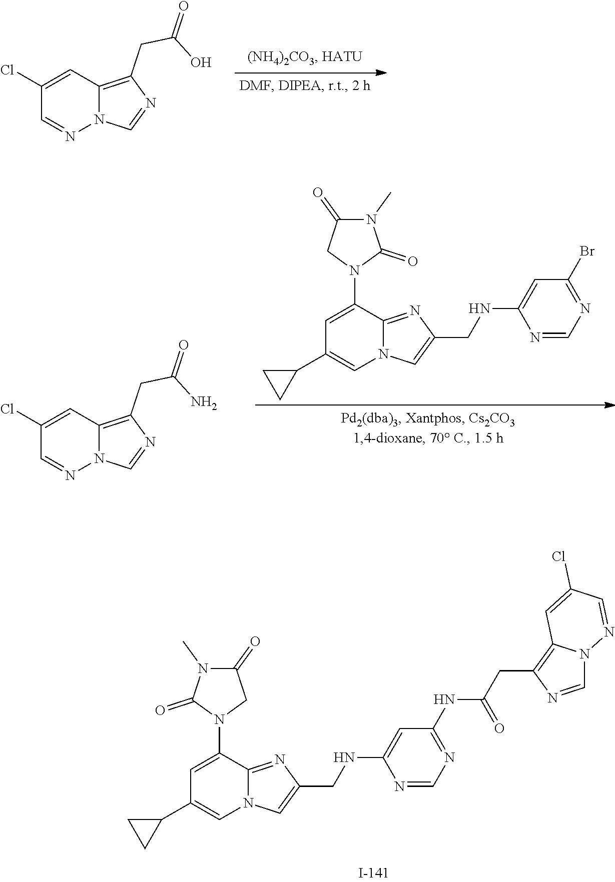 [SMILES: O=C(O)Cc1ncn2ncc(Cl)cc12 ; NC(=O)Cc1ncn2ncc(Cl)cc12 ; CN1C(=O)CN(c2cc(C3CC3)cn3cc(CNc4cc(Br)ncn4)nc23)C1=O ; CN1C(=O)CN(c2cc(C3CC3)cn3cc(CNc4cc(NC(=O)Cc5ncn6ncc(Cl)cc56)ncn4)nc23)C1=O]

Synthesis of 2-(3-chloroimidazo[1,5-b]pyridazin-5-yl)acetamide. A mixture of 2-(3-chloroimidazo[1,5-b]pyridazin-5-yl)acetic acid (140 mg, 0.66 mmol), DIPEA (428 mg, 3.30 mmol), HATU (504 mg, 1.33 mmol) and (NH₄)₂CO₃ (96 mg, 1.0 mmol) in DMF (5 mL) was stirred at room temperature for 2 h. H₂O (20 mL) was added and extracted with EtOAc (25 mL×3). The combined organics were washed with brine (30 mL), dried over Na₂SO₄, and concentrated in vacuo. The residue was purified by Prep-TLC, eluting with 10% MeOH in DCM to give 2-(3-chloroimidazo[1,5-b]pyridazin-5-yl)acetamide (80 mg, yield: 58%) as a white solid. ESI-MS [M+H]+: 211.0.

Synthesis of 2-(3-chloroimidazo[1,5-b]pyridazin-5-yl)-N-(6-(((6-cyclopropyl-8-(3-methyl-2,4-dioxoimidazolidin-1-yl)imidazo[1,2-a]pyridin-2-yl)methyl)amino)pyrimidin-4-yl)acetamide. A mixture of 2-(3-chloroimidazo[1,5-b]pyridazin-5-yl)acetamide (80 mg, 0.38 mmol), 1-(2-(((6-bromopyrimidin-4-yl)amino)methyl)-6-cyclopropylimidazo[1,2-a]pyridin-8-yl)-3-methylimidazolidine-2,4-dione (182 mg, 0.40 mmol), CS₂CO₃ (497 mg, 1.52 mmol), Pd₂(dba)₃ (36 mg, 0.04 mmol) and Xantphos (44 mg, 0.08 mmol) in 1,4-dioxane (8.0 mL) was stirred at 70° C. for 1 h under N₂ atmosphere. The reaction mixture was cooled to room temperature, filtered through a pad of Celite® and the filter cake was washed with DCM/MeOH (V/V=10/1, 100 mL). The filtrate was concentrated in vacuo. The residue was purified by Prep-TLC, eluting with 5% MeOH in DCM to give 2-(3-chloroimidazo[1,5-b]pyridazin-5-yl)-N-(6-(((6-cyclopropyl-8-(3-methyl-2,4-dioxoimidazolidin-1-yl)imidazo[1,2-a]pyridin-2-yl)methyl)amino)pyrimidin-4-yl)acetamide (25.6 mg, yield: 11%) as a white solid. ESI-MS [M+H]⁺: 586.1. 1H NMR (400 MHz, DMSO) δ10.50 (s, 1H), 8.66 (s, 1H), 8.42 (s, 1H), 8.33 (d, J=2.4 Hz, 1H), 8.24-8.20 (m, 2H), 7.87 (s, 1H), 7.65 (s, 1H), 7.31 (s, 1H), 7.24 (s, 1H), 4.87 (s, 2H), 4.56 (s, 2H), 3.97 (s, 2H), 2.95 (s, 3H), 1.95-1.88 (m, 1H), 0.94-0.90 (m, 2H), 0.64-0.55 (m, 2H).

### Example 142

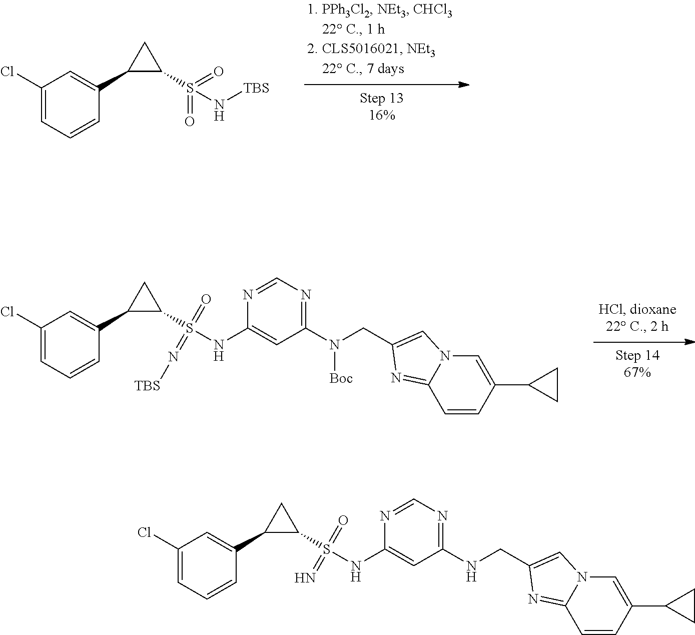

Synthesis of (RS,1SR,2RS)—N′-(tert-butyldimethylsilyl)-2-(3-chlorophenyl)-N-(6-(((6-cyclopropylimidazo[1,2-a]pyridin-2-yl)methyl)(tert-butoxycarbonyl)amino)pyrimidin-4-yl)cyclopropane-1-sulfonimidamide. Dichlorotriphenylphosphorane was freshly prepared: To a solution of triphenylphosphine oxide (464 mg, 1.66 mmol) in dry chloroform (10 mL) was added oxalyl chloride (142 μL, 1.66 mmol) dropwise over 5 min. The resulting solution was stirred for 20 min. (1SR,2RS)—N-(tert-butyldimethylsilyl)-2-(3-chlorophenyl)cyclopropane-1-sulfonamide (522 mg, 1.51 mmol) was dissolved in a mixture of dry chloroform (5 mL) and triethylamine (314 μL, 2.26 mmol). The solution of dichlorotriphenylphosphorane (as prepared above, 10 mL, 1.66 mmol) was added dropwise over 5 min and the resulting solution was stirred for 1 h at 22° C. Tert-butyl (6-aminopyrimidin-4-yl)((6-cyclopropylimidazo [1,2-a]pyridin-2-yl)methyl)carbamate (706 mg, 1.86 mmol) was added in one portion, followed by triethylamine (314 μL, 2.26 mmol). The resulting solution was stirred at room temperature for 7 days. The solvent was evaporated and the mixture was purified by reversed phase chromatography (Biotage SFaer, Cis, 60 g, 50 mL/min) with a gradient of water:acetonitrile=85:15 (1 column volume), then a gradient of water:acetonitrile=85:15 to 0:100 over 13 column volumes followed by 100% acetonitrile for 6 column volumes. The desired material eluted as a very broad peak (17-20 column volumes). Product fractions were pooled to obtain (RS,1SR,2RS)—N′-(tert-butyldimethylsilyl)-2-(3-chlorophenyl)-N-(6-(((6-cyclopropylimidazo[1,2-a]pyridin-2-yl)methyl)(tert-butyloxycarbonyl)amino)pyrimidin-4-yl)cyclopropane-1-sulfonimidamide (174 mg), as light purple foam. LCMS [System 2, Buffered 8 min method] RT=4.47 min [M+H]⁺ 708.7/710.6. ¹H NMR (400 MHz, CDCl₃, ppm) δ 8.50 (s, 1H), 8.02 (br s, 1H), 7.77 (s, 1H), 7.41 (d, J3 Hz, 1H), 7.22 (s, 1H), 7.18-7.13 (m, 2H), 7.07 (s, 1H), 6.99-6.95 (m, 1H), 6.87 (d, J10 Hz, 1H), 5.41 (s, 2H), 3.03-2.99 (m, 1H), 2.80-2.73 (m, 1H), 1.89-1.82 (m, 2H), 1.48-1.38 (m, 12H), 0.96-0.90 (m, 13H), 0.66-0.62 (m, 2H), 0.17-0.10 (m, 6H).

### Synthesis of (SR,1SR,2RS)-2-(3-chlorophenyl)-N-(6-(((6-cyclopropylimidazo[1,2-a]pyridin-2-yl)methyl)amino)pyrimidin-4-yl)cyclopropane-1-sulfonimidamide

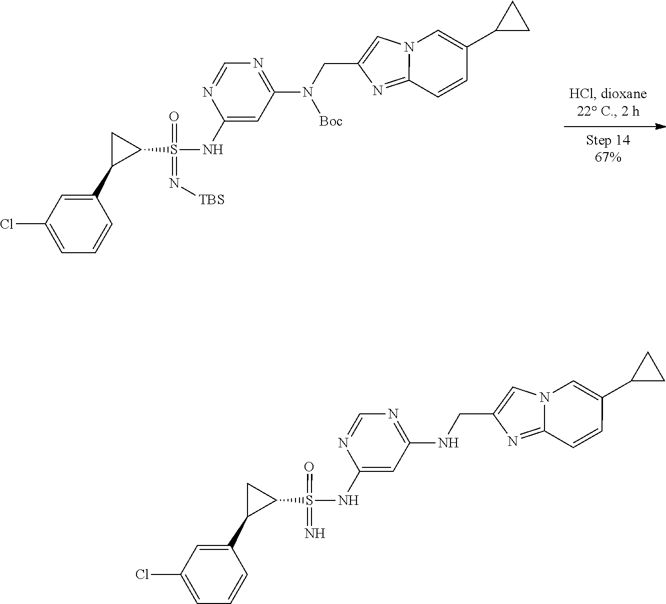

(RS,1SR,2RS)—N′-(tert-butyldimethylsilyl)-2-(3-chlorophenyl)-N-(6-(((6-cyclopropylimidazo[1,2-a]pyridin-2-yl)methyl)(tert-butyloxycarbonyl)amino)pyrimidin-4-yl)cyclopropane-1-sulfonimidamide (171 mg, 0.24 mmol) was dissolved in a solution of hydrogen chloride in dioxane (4M, 5 mL). The resulting solution was stirred at 22° C. for 2 h. A pre-prepared biphasic mixture of dichloromethane (20 mL), methanol (5 mL), and water (15 mL) containing sodium bicarbonate (4 g) was added, and the solids were dissolved with the aid of sonication. The layers were separated, and the aqueous layer was extracted with dichloromethane:methanol=95:5 (2×25 mL). The combined organic solutions were dried (Na₂SO₄), filtered and concentrated under reduced pressure. Purification on silica gel using a gradient of dichloromethane:methanol=95:5-9:1 afforded slightly contaminated product (ca 100 mg) as a 9:1 mixture of diastereomers.

This material was re-purified using reversed phase chromatography (Biotage SNAP Ultra Cis, 12 g cartridge, 12 mL/min) eluting with a gradient of 100 mM aqueous ammonium formate (A) and acetonitrile (B): 25% B (0-0.5 column volumes) followed a gradient 25-50% B over 14.5 column volumes. Partial separation of the two diastereomers was achieved to afford (SR,1SR,2RS)-2-(3-chlorophenyl)-N-(6-(((6-cyclopropylimidazo[1,2-a]pyridin-2-yl)methyl)amino)pyrimidin-4-yl)cyclopropane-1-sulfonimidamide (dr=98.7:1.3, colourless solid, 41 mg) after extractive workup (saturated aqueous sodium bicarbonate/dichloromethane).

LCMS [System 2, 4.5 min buffered] RT=2.67 min, (major isomer) [M+H]⁺ 494/496; RT=2.72 min, (minor isomer) [M+H]⁺ 494/496.

LC [System 3, Sulfons_buffered] RT=6.00 min (major isomer); RT=6.58 min (minor isomer). ¹H NMR (400 MHz, CD₃SOCD₃, ppm) major isomer only δ8.29 (s, 1H), 8.02 (s, 1H), 7.59 (s, 1H), 7.36 (d, J9 Hz, 1H), 7.32-7.24 (m, 4H), 7.17 (dt, J7, 2 Hz, 1H), 7.06 (br s, 2H), 6.95 (dd, J10, 2 Hz, 1H), 5.79 (br s, 1H), 4.46 (br s, 2H), 3.16-3.12 (m, 1H), 2.79-2.74 (m, 1H), 1.93-1.87 (m, 1H), 1.65-1.54 (m, 2H), 0.93-0.88 (m, 2H), 0.67-0.63 (m, 2H).

### Synthesis of (1SR,2RS)—N-(tert-butyldimethylsilyl)-2-(3-chlorophenyl)cyclopropane-1-sulfonamide

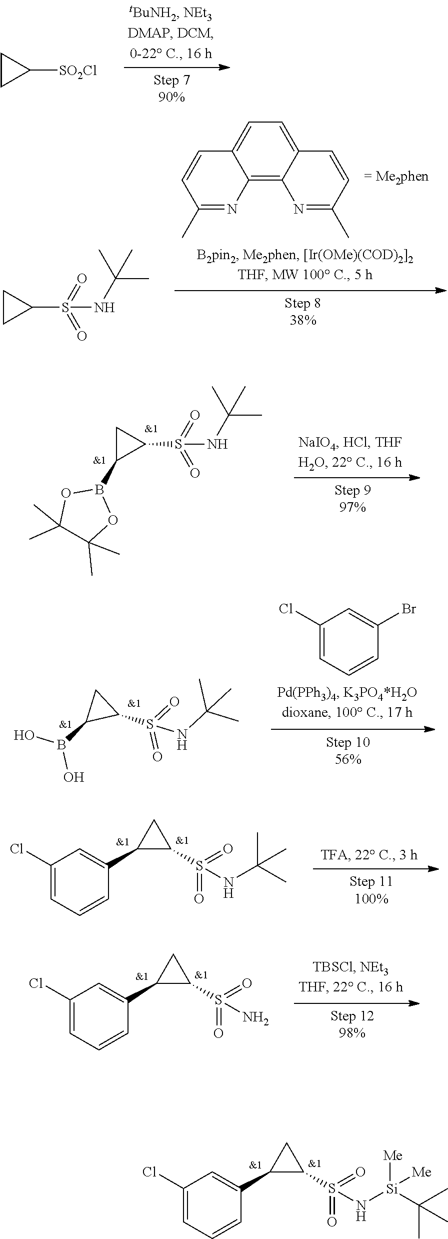

Synthesis of N-(tert-butyl)cyclopropanesulfonamide. Tert-butylamine (7.22 ml, 68.7 mmol) was added to a solution of cyclopropylsulfonyl chloride (4.83 g, 34.4 mmol), 4-dimethylaminopyridine (420 mg, 3.44 mmol) and triethylamine (9.53 ml, 68.7 mmol) in dichloromethane (140 mL) at 0° C. After being stirred for 30 min, the mixture was warmed to room temperature and stirred overnight. The solvent was removed under reduced pressure and the residue taken up in ethyl acetate (100 mL). The solution was washed with aqueous citric acid (1M, 3×50 mL). The combined aqueous layers were extracted with ethyl acetate (3×30 mL) and the combined organic solutions were washed with saturated aqueous sodium bicarbonate, followed by brine. The solution was dried (MgSO₄), filtered and concentrated under reduced pressure to obtain N-(tert-butyl)cyclopropanesulfonamide (5.47 g, 90%), as light yellow solid. LCMS [System 2, 4.5 min buffered] RT=2.17 min (by MS not uv detection), [M+H]⁺ 178. ¹HNMR (400 MHz, CDCl₃, ppm) δ 4.10 (br s, 1H), 2.49-2.42 (m, 1H), 1.39 (s, 9H), 1.21-1.16 (m, 2H), 1.02-0.96 (m, 2H).

Synthesis of (1S*,2S*)—N-(tert-butyl)-2-(4,4,5,5-tetramethyl-1,3,2-dioxaborolan-2-yl)cyclopropane-1-sulfonamide. A dry, 30 mL microwave vial was charged with N-(tert-butyl)cyclopropanesulfonamide (1.00 g, 5.64 mmol), bis(pinacolato)diboron (2.15 g, 8.46 mmol), 2,9-dimethyl[1,10]phenanthroline (Me₂phen, 117 mg, 0.56 mmol) and (1,5-cyclooctadiene)iridium (I) methoxide dimer ([Ir(COD)(OMe)]2,187 mg, 0.28 mmol). The mixture was evacuated and backfilled with nitrogen three times before anhydrous tetrahydrofuran (15 mL) was added. The degassing process was repeated and the solution was placed in a microwave reactor at 100° C. for 5 h. Silica gel (5 g) was added, the mixture was concentrated under reduced pressure and the residue was loaded on a silica gel column equilibrated with heptane:ethyl acetate=95:5. Elution with a gradient of heptane:ethyl acetate=95:5-75:25 afforded (1S*,2S*)—N-(tert-butyl)-2-(4,4,5,5-tetramethyl-1,3,2-dioxaborolan-2-yl)cyclopropane-1-sulfonamide (1.96 g), as a golden syrup which consisted as a mixture with starting material and bis(pinacolato)diboron, 38% yield). This was used in the next step without further purification. LC-MS [System 2, 4.5 min buffered] RT=2.98 min (by MS not uv detection), [M+H]⁺ 304. ¹H NMR (400 MHz, CDCl₃, ppm) δ 4.10 (br s, 1H), 2.63-2.58 (m, 1H), 1.38 (s, 9H), 1.27 (s, 12H), 1.02-0.97 (m, 1H), 0.77-0.71 (m, 1H).

Synthesis of ((1S*,2S*)-2-(N-(tert-butyl)sulfamoyl)cyclopropyl)boronic acid. Sodium metaperiodate (11.4 g, 53.3 mmol) was added to a solution of (1S*,2S*)—N-(tert-butyl)-2-(4,4,5,5-tetramethyl-1,3,2-dioxaborolan-2-yl)cyclopropane-1-sulfonamide (1.96, 33% pure, ˜ 2.14 mmol) in a mixture of tetrahydrofuran (40 mL) and water (10 mL) at room temperature. Hydrochloric acid (2 M, 2.66 mL, 5.33 mmol) was added and the mixture was stirred overnight. The pH was brought to 8 by the addition of saturated aqueous sodium bicarbonate and the organic solvent was evaporated under reduced pressure. The residue was taken up in ethyl acetate (30 mL) and water (30 mL). Inorganic salts were removed by filtration, and the filtrate layers were separated. The aqueous layer was extracted with ethyl acetate (20 mL). The combined organic layers were concentrated under reduced pressure and the residue taken up in methyl tert-butyl ether (30 mL). This solution was extracted with aqueous sodium hydroxide (2 M, 3×10 mL). The combined aqueous layers were washed with methyl tert-butyl ether (10 mL). The pH was brought to 1 by the slow addition of concentrated hydrochloric acid (32% w/v, ca 7 mL). The product was extracted into ethyl acetate (3×15 mL). The combined organic extracts were dried (Na₂SO₄), filtered and concentrated to obtain ((1S*,2S*)-2-(N-(tert-butyl)sulfamoyl) cyclopropyl)boronic acid (458 mg, 97%), as a light brown oil that crystallised upon standing. LCMS [System 2, 4.5 min buffered] RT=1.67 min (by MS not uv detection), [M−H₂O—H]⁻202. ¹H NMR (400 MHz, CD₃SOCD₃, ppm) δ 7.81 (s, 2H), 6.80 (s, 1H), 2.50-2.40 (m, 1H), 1.26 (s, 9H), 1.08-1.02 (m, 1H), 0.93-0.88 (m, 1H), 0.48-0.42 (m, 1H).

Synthesis of (1S*,2S*)—N-(tert-butyl)-2-(3-chlorophenyl)cyclopropane-1-sulfonamide. A two-necked flask was charged with potassium phosphate hydrate (2.23 g, 9.70 mmol), a solution of ((1S*,2S*)-2-(N-(tert-butyl)sulfamoyl)cyclopropyl)boronic acid (794 mg, 3.23 mmol) in dioxane (160 mL) and 3-chloro bromobenzene (759 μL, 6.46 mmol). The mixture was degassed (vacuum/nitrogen backfill×3) and tetrakis-triphenylphosphine palladium (0) (374 mg, 0.323 mmol) was added. The mixture was degassed again and was heated to 100° C. overnight. The mixture was allowed to cool to room temperature and filtered through a pad of Dicalite, washing the filter cake with ethyl acetate. The filtrate was combined with three smaller batches (each from 50 mg (0.2 mmol) of the boronic acid, treated in the same manner) and concentrated under reduced pressure. The residue was purified on a silica gel column using a gradient of heptane:ethyl acetate=90:10-85:15 to afford the product as a mixture with triphenylphosphine oxide (940 mg). Further purification by reversed phase chromatography (Biotage Cis SNAP Ultra, 30 g) using a gradient of water:acetonitrile=90:10-10:90 over 10 column volumes. Fractions containing the product were pooled and concentrated under reduced pressure to obtain (1S*,2S*)—N-(tert-butyl)-2-(3-chlorophenyl)cyclopropane-1-sulfonamide (691 mg, 56%), as a light yellow oil. LC-MS [System 2, 4.5 min buffered] RT=3.27 min, [M−H]⁻286/288. ¹H NMR (400 MHz, CD₃SOCD₃, ppm) δ 7.34-7.19 (m, 4H), 7.00 (s, 1H), 2.93-2.88 (m, 1H), 2.58-2.51 (m, 1H), 1.56-1.48 (m, 2H), 1.24 (s, 9H).

Synthesis of (1SR,2RS)-2-(3-chlorophenyl)cyclopropane-1-sulfonamide. A solution of (1S*,2S*)—N-(tert-butyl)-2-(3-chlorophenyl)cyclopropane-1-sulfonamide (688 mg, 2.39 mmol) in trifluoroacetic acid (5 mL) was stirred at room temperature for 2 h. The mixture was concentrated to dryness and taken up in fresh trifluoroacetic acid (10 mL). After 1 h complete conversion to product was observed and the solvent was evaporated. The oily residue evaporated several times from acetonitrile to obtain (1S*,2S*)-2-(3-chlorophenyl)cyclopropane-1-sulfonamide (700 mg, 126%, remainder TFA; 79% w/w product), as a white solid. LC-MS [System 2, 4.5 min buffered] RT=2.54 min, [M−H]⁻230/232. ¹H NMR (400 MHz, CD₃SOCD₃, ppm) δ 7.34-7.25 (m, 3H), 7.19 (d, J8 Hz, 1H), 6.97 (br s, 2H), 2.89-2.84 (m, 1H), 2.54 (hidden by DMSO, m, 1H), 1.54-1.47 (m, 2H).

Synthesis of (1S*,2S*)—N-(tert-butyldimethylsilyl)-2-(3-chlorophenyl)cyclopropane-1-sulfonamide. (1S*,2S*)-2-(3-chlorophenyl)cyclopropane-1-sulfonamide (79% w/w, 600 mg, 2.05 mmol) was dissolved in a mixture of tetrahydrofuran (3 mL) and triethylamine (567 μL, 4.09 mmol) at room temperature. A solution of tert-butyldimethylsilyl chloride (617 mg, 4.09 mmol) in tetrahydrofuran (3 mL) was added, and the resulting white suspension was stirred overnight. The mixture was concentrated onto silica gel and purified on a silica gel column using a gradient of heptane:ethyl acetate=9:1-7:3) to afford (1S*,2S*)—N-(tert-butyldimethylsilyl)-2-(3-chlorophenyl)cyclopropane-1-sulfonamide (696 mg, 98%), as a colourless solid. LC-MS [System 2, 4.5 min buffered] RT=3.82 min, [M+H]⁺ 346/348. ¹H NMR (400 MHz, CDCl₃, ppm) δ 7.25-7.19 (m, 2H), 7.11-7.08 (m, 1H), 7.04-7.01 (m, 1H), 4.05 (s, 1H), 2.66 (t, J8 Hz, 2H), 1.75-1.70 (m, 1H), 1.43-1.38 (m, 1H), 0.94 (s, 9H), 0.30 (s, 6H).

### Example 143

### Inhibitory Activity of Exemplary Compounds Against Plasma Kallikrein.

Compounds were evaluated for inhibition of the human activated kallikrein enzyme in two formats of an assay employing a fluorogenic peptide substrate. In one assay format, the concentrations of reagents were as follows: 20 mM Tris pH 7.5, 1 mM EDTA, 150 mM sodium chloride, 0.1% PEG-400, 0.1% Triton X-100, 500 pM activated kallikrein enzyme, 300 μM Pro-Phe-Arg-7-amido-4-methylcoumarin (PFR-AMC) substrate. Prior to reaction initiation with substrate, enzyme and inhibitors were preincubated for 30 min at room temperature. After initiation with substrate, reactions were incubated for 10 min at room temperature and fluorescence emission at 460 nm from 380 nm excitation measured with a microplate reader. In another assay format, the concentrations of reagents were as follows: 20 mM Tris pH 7.5, 1 mM EDTA, 150 mM sodium chloride, 0.1% PEG-400, 0.1% Triton X-100, 5 pM activated kallikrein enzyme, 300 uM PFR-AMC substrate. Prior to reaction initiation with substrate, enzyme and inhibitors were preincubated for 30 min at room temperature. After initiation with substrate, reactions were incubated for 18 hr at room temperature and fluorescence emission at 460 nm from 380 nm excitation measured with a microplate reader.

Table 1 provides the results of the assay in the format with 500 pM activated kallikrein assay. For the compounds listed in Table 1, the EC₅₀ values are reported according to the following ranges: A≤5.0 nM; 5.0 nM<B≤50 nM; 50 nM<C≤500 nM; 500 nM<D≤9000 nM; 9000 nM<E.

TABLE 1

| | Primary Dose |
|---|---|
| | Response |
| | Assay: Average |
| Compound | EC50 |
| 1 | A |
| 2 | D |
| 3 | D |
| 4 | B |
| 5 | D |
| 6 | C |
| 7 | D |
| 8 | D |
| 9 | D |
| 10 | E |
| 11 | D |
| 12 | A |
| 13 | A |
| 14 | A |
| 15 | A |
| 16 | A |
| 17 | A |
| 18 | A |
| 19 | A |
| 20 | A |
| 21 | A |
| 22 | B |
| 23 | B |
| 24 | B |
| 25 | A |
| 26 | A |
| 27 | D |
| 28 | D |
| 29 | D |
| 30 | A |
| 31 | A |
| 32 | A |
| 33 | B |
| 34 | C |
| 35 | B |
| 36 | D |
| 37 | A |
| 38 | A |
| 39 | A |
| 40 | A |
| 41 | B |
| 42 | C |
| 43 | B |
| 44 | A |
| 45 | B |
| 46 | A |
| 47 | B |
| 48 | A |
| 49 | D |
| 50 | B |
| 51 | E |
| 52 | A |
| 53 | A |
| 54 | A |
| 55 | A |
| 56 | B |
| 57 | A |
| 58 | D |
| 59 | A |
| 60 | C |
| 61 | D |
| 62 | D |
| 63 | B |
| 64 | B |
| 65 | B |
| 66 | B |
| 67 | D |
| 68 | B |
| 69 | A |
| 70 | A |
| 71 | B |
| 72 | A |
| 73 | C |
| 74 | A |
| 75 | C |
| 76 | C |
| 77 | B |
| 81 | C |
| 82 | A |
| 83 | C |
| 84 | B |
| 85 | A |
| 86 | C |
| 87 | C |
| 88 | A |
| 90 | D |
| 91 | A |
| 92 | A |
| 93 | A |
| 94 | A |
| 95 | A |
| 96 | A |
| 97 | A |
| 98 | A |
| 99 | A |
| 100 | B |
| 101 | B |
| 102 | A |
| 103 | A |
| 104 | E |
| 105 | B |
| 106 | E |
| 107 | A |
| 108 | A |
| 109 | A |
| 110 | A |
| 111 | A |
| 112 | A |
| 113 | A |
| 114 | B |
| 115 | D |
| 116 | B |
| 117 | B |
| 118 | C |
| 119 | E |
| 120 | D |
| 121 | E |
| 122 | E |
| 123 | D |
| 124 | D |
| 125 | E |
| 126 | D |
| 127 | D |
| 128 | D |
| 129 | C |
| 130 | A |
| 131 | D |
| 132 | B |
| 133 | E |
| 134 | D |
| 135 | A |
| 136 | D |
| 137 | C |
| 138 | D |
| 139 | C |
| 140 | D |
| 141 | A |
| 142 | D |

### Example 144

### Neat Human and Rat Plasma Assay (Fluorogenic Peptide).

To analyze inhibition of plasma kallikrein in an ex vivo setting, the potency of compounds was measure in contact pathway-activated plasma assays. In a fluorogenic peptide substrate assay, test compounds dissolved in DMSO were added to sodium citrate collected human or rat plasma in a 96-well microplate. Alternatively, citrated plasma was collected from rats administered the compounds orally or by IV. 10 nM of human FXIIa (Enzyme Research Laboratories) diluted in PKa buffer (20 mM Tris-HCl, pH 7.5, 150 mM NaCl, 1 mM EDTA, 0.1% PEG-8000, and 0.1% Triton X-100) was added to the plasma, followed by the 100 μM of the profluorescent, synthetic plasma kallikrein substrate PFR-AMC (also diluted in PKa buffer). Final plasma concentration in the reaction was 78%. Fluorescence was immediately monitored by excitation/emission wavelengths of 360 nm/480 nm respectively over a period of 5 minutes in a microplate reader. The resulting linear increase in fluorescence emission (reflecting PKa proteolysis of PFR-AMC substrate) was fit to extract a proteolytic rate (fluorescent units over time), and this rate was subsequently plotted against compound inhibitor concentration. Resulting plots were fit to a standard 4-parameter IC50/IC90 equation to determine min/max values, IC50/90, and slope. All experimental steps were performed at room temperature. Table 2 provides results of the assay.

For the compounds listed in Table 2, the IC₉₀ values are reported according to the following ranges: A≤500 nM; 500 nM<B≤5000 nM; 5000 nM<C≤25000 nM.

TABLE 2

| | Human Plasma |
|---|---|
| Compound | Assay: IC90 |
| 1 | B |
| 12 | B |
| 14 | A |
| 17 | A |
| 26 | C |
| 30 | B |
| 31 | B |
| 52 | A |
| 55 | B |
| 107 | C |
| 109 | A |
| 110 | C |
| 112 | B |
| 113 | C |
| 135 | A |
| 141 | B |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of examples.

### Exemplary Enumerated Embodiments

1. A compound of formula I:

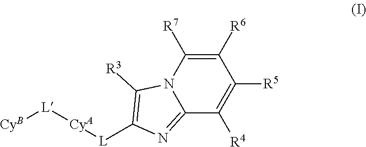

or a pharmaceutically acceptable salt thereof,
wherein:
Cy^{A} is a 4-membered monocyclic carbocyclene, a 3- to 7-membered saturated or partially unsaturated monocyclic heterocyclene having 1-3 heteroatoms selected from oxygen, nitrogen, or sulfur, phenylene, a 5- to 6-membered monocyclic heteroarylene having 1-3 heteroatoms independently selected from oxygen, nitrogen, and sulfur, a 7- to 10-membered saturated or partially unsaturated bicyclic heterocyclene having 1-4 heteroatoms selected from oxygen, nitrogen, or sulfur, or an 8- to 12-membered bicyclic heteroarylene having 1-4 heteroatoms independently selected from oxygen, nitrogen, and sulfur, wherein Cy^{A} is substituted with 0-4-R^{A} groups;
   each R^{A} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, 3- to 7-membered saturated or partially unsaturated monocyclic carbocyclyl, 3- to 7-membered saturated or partially unsaturated monocyclic heterocyclyl having 1-2 heteroatoms selected from oxygen, nitrogen, or sulfur, or a 5- to 6-membered monocyclic heteroaryl having 1-4 heteroatoms independently selected from oxygen, nitrogen or sulfur;
each R is independently hydrogen or an optionally substituted C₁₋₆ aliphatic group;
Cy^{B} is selected from phenyl, 8- to 10-membered bicyclic aryl, 7- to 10-membered saturated or partially unsaturated bicyclic carbocycyl, a 5- to 6-membered heteroaryl having 1-3 heteroatoms independently selected from oxygen, nitrogen, and sulfur, a 7- to 10-membered saturated or partially unsaturated bicyclic heterocycyl having 1-3 heteroatoms selected from oxygen, nitrogen, or sulfur, or a 7- to 10-membered heteroaryl having 1-4 heteroatoms independently selected from oxygen, nitrogen, and sulfur, wherein Cy^{B} is substituted with 0-5-R^{B} groups; or
   each R^{B} is independently selected from oxo, halogen, —CN, —C(O)R, —C(O)₂R, —C(O)N(R)₂, —C(NR)NR₂, —C(NR)NROR, —C(NR)NRC(O)OR, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, a 3- to 7-membered saturated or partially unsaturated monocyclic heterocyclyl having 1-2 heteroatoms selected from oxygen, nitrogen, or sulfur, or a 5- to 6-membered heteroaryl having 1-4 heteroatoms independently selected from oxygen, nitrogen, and sulfur;
L′ is a covalent bond or an optionally substituted C₁₋₄ hydrocarbon chain, wherein 1 to 3 methylene units are optionally and independently replaced with —O—, —C(O)—, —NR^{z}—, —S—, —SO—, SO₂—, —S(NH)(O)—, or cyclopropylene;
each R^{z} is independently selected from hydrogen, —(CH₂)₀₋₃OR, —(CH₂)₀₋₃C(O)OR, or an optionally substituted C₁₋₆ aliphatic group;
L is an optionally substituted C₁₋₂ hydrocarbon chain, wherein 1 methylene unit is optionally and independently replaced with —C(O)—, —O—, —NR—, —S—, —SO—, or —SO₂—; or L is an optionally substituted 5- to 6-membered saturated or partially unsaturated heterocyclene, having 1-3 heteroatoms independently selected from oxygen, nitrogen, and sulfur;
each R³, R⁴, R⁵, R⁶, and R⁷ is independently selected from hydrogen or -L^{C}-R^{C}, wherein
   each L^{C} is independently selected from a covalent bond or an optionally substituted C₁₋₆ hydrocarbon chain, wherein 1 to 3 methylene units are optionally and independently replaced with —O— or —NR—;
   each R^{C} is independently selected from halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, Cy^{C}, or an optionally substituted C₁₋₆ aliphatic; and
each Cy^{C} is an optionally substituted ring independently selected from a 3- to 7-membered saturated or partially unsaturated monocyclic carbocyclyl, a 3- to 7-membered saturated or partially unsaturated monocyclic heterocyclyl having 1-2 heteroatoms selected from oxygen, nitrogen, or sulfur, a 5- to 6-membered monocyclic heteroaryl having 1-3 heteroatoms independently selected from oxygen, nitrogen, and sulfur, phenyl, a 6- to 12-membered saturated or partially unsaturated fused bicyclic heterocyclyl having 1-3 heteroatoms independently selected from oxygen, nitrogen, or sulfur, a bridged bicycle, or a 6- to 12-membered saturated or partially unsaturated bicyclic spiroheterocyclyl having 1-3 heteroatoms independently selected from oxygen, nitrogen, or sulfur.
2. The compound of embodiment 1, provided that:
when:
   i) Cy^{B} is

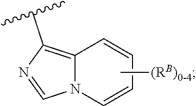

or
ii) Cy^{A} is a 5-membered monocyclic heteroarylene having 1-3 heteroatoms independently selected from oxygen, nitrogen, and sulfur, wherein Cy^{A} is substituted with 0-4-R^{A} groups;
then:
   L is —C(O)— or an optionally substituted C₂ hydrocarbon chain, wherein 1 methylene unit is optionally and independently replaced with —O— or —NR^{z}—; or L is an optionally substituted 5-membered saturated or partially unsaturated heterocyclene, having 1-3 heteroatoms independently selected from oxygen, nitrogen, and sulfur.
3. The compound of embodiment 1 or 2, provided that:
when:
   Cy^{B} is

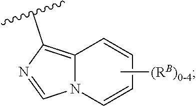

then:
   L is —C(O)— or an optionally substituted C₂ hydrocarbon chain, wherein 1 methylene unit is optionally and independently replaced with —O— or —NR^{z}—; or L is an optionally substituted 5-membered saturated or partially unsaturated heterocyclene, having 1-3 heteroatoms independently selected from oxygen, nitrogen, and sulfur.
4. The compound of any of the preceding embodiments, provided that
when:
   Cy^{A} is a 5-membered monocyclic heteroarylene having 1-3 heteroatoms independently selected from oxygen, nitrogen, and sulfur, wherein Cy^{A} is substituted with 0-4-R^{A} groups;
then:
   L is —C(O)— or an optionally substituted C₂ hydrocarbon chain, wherein 1 methylene unit is optionally and independently replaced with —O— or —NR^{z}—; or L is an optionally substituted 5-membered saturated or partially unsaturated heterocyclene, having 1-3 heteroatoms independently selected from oxygen, nitrogen, and sulfur.
5. The compound of any of the preceding embodiments, wherein Cy^{A} is a 4-membered monocyclic carbocyclene, a 3- to 7-membered saturated or partially unsaturated monocyclic heterocyclene having 1-3 heteroatoms selected from oxygen, nitrogen, or sulfur, a 5- to 6-membered monocyclic heteroarylene having 1-3 heteroatoms independently selected from oxygen, nitrogen, and sulfur, a 7- to 10-membered saturated or partially unsaturated bicyclic heterocyclene having 1-4 heteroatoms selected from oxygen, nitrogen, or sulfur, or a 7- to 10-membered bicyclic heteroarylene having 1-4 heteroatoms independently selected from oxygen, nitrogen, and sulfur, wherein Cy^{A} is substituted with 0-4-R^{A} groups.
6. The compound of any of the preceding embodiments, wherein Cy^{A} is a 4-membered monocyclic carbocyclene, wherein Cy^{A} is substituted with 0-4-R^{A} groups.
7. The compound of any of the preceding embodiments, wherein Cy^{A} is cyclobutendionediyl, wherein Cy^{A} is substituted with 0-2-R^{A} groups.
8. The compound of any one of embodiments 1-5, wherein Cy^{A} is a 5- to 6-membered monocyclic heteroarylene having 1-3 heteroatoms independently selected from oxygen, nitrogen, and sulfur, wherein Cy^{A} is substituted with 0-4-R^{A} groups.
9. The compound of any one of embodiments 1-5 or 8, wherein Cy^{A} is a 6-membered monocyclic heteroarylene having 1-3 nitrogen heteroatoms, wherein Cy^{A} is substituted with 0-4 R^{A} groups.
10. The compound of any one of embodiments 1-5 or 8-9, wherein Cy^{A} is a pyridinediyl, pyrimidinediyl, or pyridazinediyl, substituted with 0-3 R^{A} groups.
11. The compound of any one of embodiments 1-5 or 8, wherein Cy^{A} is a 5-membered monocyclic heteroarylene having 1-3 heteroatoms independently selected from oxygen, nitrogen, and sulfur, wherein Cy^{A} is substituted with 0-2-R^{A} groups.
12. The compound of any one of embodiments 1-5, 8, or 11, wherein Cy^{A} is a thiazolediyl, substituted with 0-3 R^{A} groups.
13. The compound of any one of embodiments 1-5, wherein Cy^{A} is 3- to 7-membered saturated or partially unsaturated monocyclic heterocyclene having 1-3 heteroatoms selected from oxygen, nitrogen, or sulfur, wherein Cy^{A} is substituted with 0-4-R^{A} groups.
14. The compound of any one of embodiments 1-5, wherein Cy^{A} is a 7- to 10-membered saturated or partially unsaturated bicyclic heterocyclene having 1-4 heteroatoms selected from oxygen, nitrogen, or sulfur, wherein Cy^{A} is substituted with 0-4-R^{A} groups.
15. The compound of any one of embodiments 1-5 or 14, wherein Cy^{A} is a 9-membered saturated or partially unsaturated bicyclic heterocyclene having 1-4 heteroatoms selected from oxygen, nitrogen, or sulfur, wherein Cy^{A} is substituted with 0-4-R^{A} groups.
16. The compound of any one of embodiments 1-5 or 14-15, wherein Cy^{A} is a 9-membered saturated or partially unsaturated bicyclic heterocyclene having 2 nitrogen heteroatoms, wherein Cy^{A} is substituted with 0-4-R^{A} groups.
17. The compound of any one of embodiments 1-5 or 14, wherein Cy^{A} is a 10-membered saturated or partially unsaturated bicyclic heterocyclene having 1-4 heteroatoms selected from oxygen, nitrogen, or sulfur, wherein Cy^{A} is substituted with 0-4-R^{A} groups.
18. The compound of any one of embodiments 1-5, 14, or 17, wherein Cy^{A} is a 10-membered saturated or partially unsaturated bicyclic heterocyclene having 2 nitrogen heteroatoms, wherein Cy^{A} is substituted with 0-4-R^{A} groups.
19. The compound of any one of embodiments 1-5 or 14, wherein Cy^{A} is dihydroindazolonediyl or quinazolinonediyl, substituted with 0-4 R^{A} groups.
20. The compound of any one of embodiments 1-5, wherein Cy^{A} is an 8- to 12-membered bicyclic heteroarylene having 1-4 heteroatoms independently selected from oxygen, nitrogen, and sulfur, wherein Cy^{A} is substituted with 0-4-R^{A} groups.
21. The compound of any one of embodiments 1-5 or 20, wherein Cy^{A} is a 9-membered bicyclic heteroarylene having 2 nitrogen heteroatoms, wherein Cy^{A} is substituted with 0-3-R^{A} groups.
21. The compound of any one of embodiments 1-5 or 20-21, wherein Cy^{A} is benzoimidazolediyl substituted with 0-4 R^{A} groups.
22. The compound of any one of embodiments 1-5, wherein Cy^{A} is selected from the group consisting of:

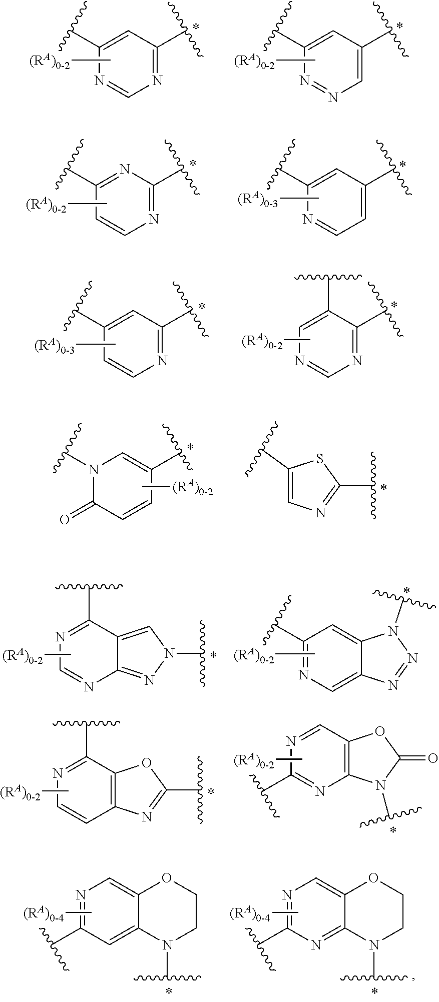

wherein * represents the point of attachment to L.
23. The compound of any one of embodiments 1-5 or 22, wherein Cy^{A} is selected from the group consisting of:

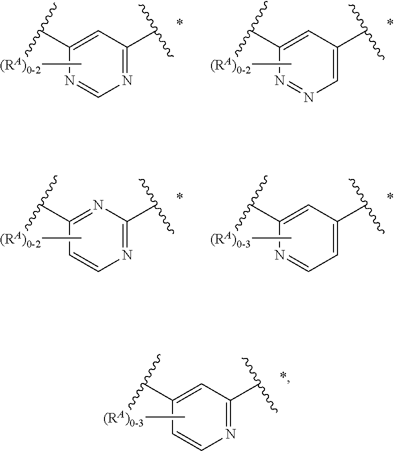

wherein * represents the point of attachment to L.
24. The compound of any one of the preceding embodiments, wherein each R^{A} is independently selected from oxo, —C(O)R, —C(O)₂R, —OR, or an optionally substituted group selected from C₁₋₆ aliphatic or a 5- to 6-membered monocyclic heteroaryl having 1-4 heteroatoms independently selected from oxygen, nitrogen or sulfur.
25. The compound of any one of the preceding embodiments, wherein substituents on an optionally substituted R^{A} group are independently halogen, —(CH₂)₀₋₄OR^{o}, or —(CH₂)₀₋₄C(O)OR^{o}, wherein each R^{o} is independently as defined above and described in classes and subclasses herein.
26. The compound of any one of the preceding embodiments, wherein Cy^{B} is phenyl, wherein Cy^{B} is substituted with 0-5-R^{B} groups.
27. The compound of any one of embodiments 1-25, wherein Cy^{B} is 5- to 6-membered heteroaryl having 1-3 heteroatoms independently selected from oxygen, nitrogen, and sulfur, wherein Cy^{B} is substituted with 0-4-R^{B} groups.
28. The compound of any one of embodiments 1-25 or 27, wherein Cy^{B} is a 6-membered heteroaryl having 1-3 heteroatoms independently selected from oxygen, nitrogen, and sulfur, wherein Cy^{B} is substituted with 0-4-R^{B} groups.
29. The compound of any one of embodiments 1-25 or 27-28, wherein Cy^{B} is a pyridinyl or pyridinonyl group substituted with 0-2-R^{B} groups.
30. The compound of any one of embodiments 1-25, wherein Cy^{B} is a 5-membered heteroaryl having 1-3 heteroatoms independently selected from oxygen, nitrogen, and sulfur, wherein Cy^{B} is substituted with 0-4-R^{B} groups.
31. The compound of any one of embodiments 1-25, wherein Cy^{B} is selected from the group consisting of:

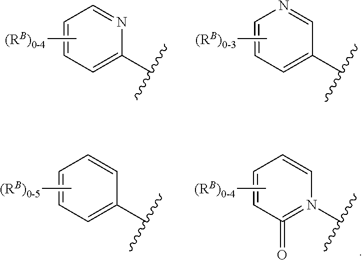

32. The compound of any one of embodiments 1-25, wherein Cy^{B} is a 8- to 10-membered bicyclic aryl, wherein Cy^{B} is substituted with 0-4-R^{B} groups.
33. The compound of any one of embodiments 1-25 or 32, wherein Cy^{B} is a 10-membered bicyclic aryl, wherein Cy^{B} is substituted with 0-4-R^{B} groups.
34. The compound of any one of embodiments 1-25 or 32-33, wherein Cy^{B} is a 1,2,3,4-tetrahydronaphthaleneyl, naphthaleneyl, or indoleyl, wherein Cy^{B} is substituted with 0-4-R^{B} groups.
35. The compound of any one of embodiments 1-25, wherein Cy^{B} is a 7- to 10-membered saturated or partially unsaturated bicyclic carbocycyl, wherein Cy^{B} is substituted with 0-4-R^{B} groups.
36. The compound of any one of embodiments 1-25 or 35, wherein Cy^{B} is a 9-membered saturated or partially unsaturated bicyclic carbocycyl, wherein Cy^{B} is substituted with 0-4-R^{B} groups.
37. The compound of any one of embodiments 1-25 or 35-36, wherein Cy^{B} is a 6,7-dihydro-5H-cyclopentapyridineyl, wherein Cy^{B} is substituted with 0-4-R^{B} groups.
38. The compound of any one of embodiments 1-25, wherein Cy^{B} is a 7- to 10-membered saturated or partially unsaturated bicyclic heterocycyl having 1-3 heteroatoms selected from oxygen, nitrogen, or sulfur, wherein Cy^{B} is substituted with 0-4-R^{B} groups.
39. The compound of any one of embodiments 1-25 or 38, wherein Cy^{B} is a 9-membered saturated or partially unsaturated bicyclic heterocycyl having 1-3 heteroatoms selected from oxygen, nitrogen, or sulfur, wherein Cy^{B} is substituted with 0-4-R^{B} groups.
40. The compound of any one of embodiments 1-25 or 38-39, wherein Cy^{B} is a benzooxazoleyl, wherein Cy^{B} is substituted with 0-4-R^{B} groups; or Cy^{B} is a benzooxazoloneyl, wherein Cy^{B} is further substituted with 0-3 additional —R^{B} groups.
41. The compound of any one of embodiments 1-25, wherein Cy^{B} is a 7- to 10-membered heteroaryl having 1-4 heteroatoms independently selected from oxygen, nitrogen, and sulfur, wherein Cy^{B} is substituted with 0-4-R^{B} groups.
42. The compound of any one of embodiments 1-25 or 41, wherein Cy^{B} is a 9-membered heteroaryl having 1-4 heteroatoms independently selected from oxygen, nitrogen, and sulfur, wherein Cy^{B} is substituted with 0-4-R^{B} groups.
43. The compound of any one of embodiments 1-25 or 41-42, wherein Cy^{B} is a 9-membered heteroaryl having 1-3 nitrogen heteroatoms, wherein Cy^{B} is substituted with 0-4-R^{B} groups.
44. The compound of any one of embodiments 1-25 or 41-43, wherein Cy^{B} is indoleyl, imidazopyridineyl, imidazopyridazineyl, benzotriazoleyl, benzoimidazoleyl, or pyrrolopyridineyl, wherein Cy^{B} is substituted with 0-4-R^{B} groups.
45. The compound of any one of embodiments 1-25 or 41, wherein Cy^{B} is a 10-membered heteroaryl having 1-4 heteroatoms independently selected from oxygen, nitrogen, and sulfur, wherein Cy^{B} is substituted with 0-4-R^{B} groups.
46. The compound of any one of embodiments 1-25, 41, or 45, wherein Cy^{B} is a 10-membered heteroaryl having 1-2 nitrogen heteroatoms, wherein Cy^{B} is substituted with 0-4-R^{B} groups.
47. The compound of any one of embodiments 1-25, 41, or 45-46, wherein Cy^{B} is quinazolineyl or phthalazineyl, wherein Cy^{B} is substituted with 0-4-R^{B} groups.
48. The compound of any one of embodiments 1-25, wherein Cy^{B} is selected from the group consisting of:

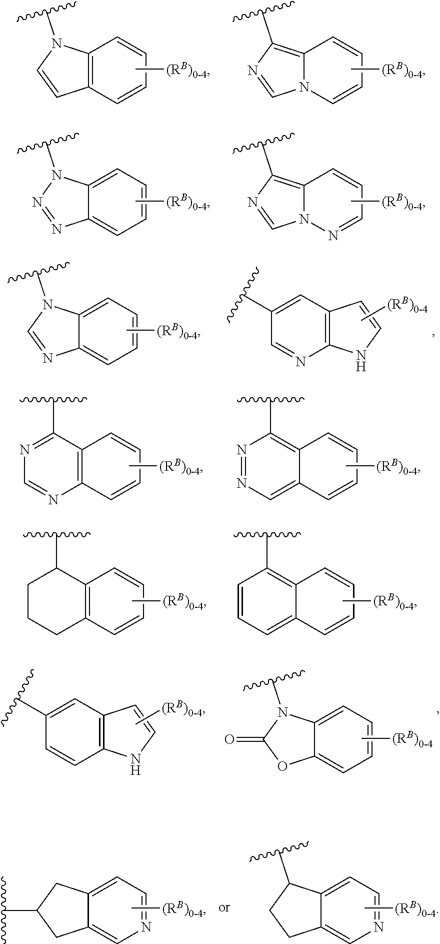

49. The compound of any one of embodiments 1-25 or 48, wherein Cy^{B} is selected from the group consisting of

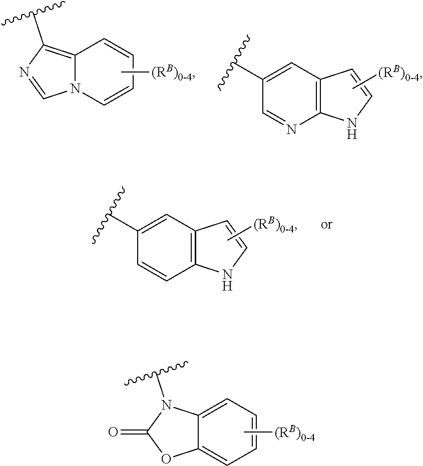

50. The compound of any one of embodiments 1-25 or 48-49, wherein Cy^{B} is

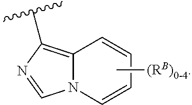

51. The compound of any one of the preceding embodiments, wherein each R^{B} is independently selected from oxo, halogen, —CN, —C(O)N(R)₂, —C(NR)NR₂, —C(NR)NROR, —C(NR)NRC(O)OR, —N(R)₂, —OR, or an optionally substituted group selected from C₁₋₆ aliphatic, or a 5- to 6-membered heteroaryl having 1-4 heteroatoms independently selected from oxygen, nitrogen, and sulfur.
52. The compound of any one of the preceding embodiments, wherein substituents on an optionally substituted R^{B} group are independently selected from oxo, halogen, —(CH₂)₀₋₄OR^{o}, —(CH₂)₀₋₄N(R^{o})₂, —(CH₂)₀₋₄C(O)NR^{o}₂, and —(CH₂)₀₋₄OC(O)R^{o}; wherein R^{o} is as defined above and described in classes and subclasses herein.
53. The compound of any one of the preceding embodiments, wherein L is an optionally substituted C₁₋₂ hydrocarbon chain, wherein 1 methylene unit is optionally replaced with —C(O)—, —O—, —NR—; or L is an optionally substituted 5- to 6-membered saturated or partially unsaturated heterocyclene, having 1-3 heteroatoms independently selected from oxygen, nitrogen, and sulfur.
54. The compound of any one of the preceding embodiments, wherein L is —C(O)— or an optionally substituted C₂ hydrocarbon chain, wherein 1 methylene unit is optionally and independently replaced with —O— or —NR—; or L is an optionally substituted 5-membered saturated or partially unsaturated heterocyclene, having 1-3 heteroatoms independently selected from oxygen, nitrogen, and sulfur.
55. The compound of any one of embodiments 1-53, wherein L is an optionally substituted C₁₋₂ hydrocarbon chain, wherein 1 methylene unit is optionally replaced with —C(O)—, —O—, —NR^{z}—.
56. The compound of any one of embodiments 1-53 or 55, wherein L is an optionally substituted C₁ hydrocarbon chain, wherein 1 methylene unit is optionally replaced with —C(O)—, —O—, —NR^{z}—.
57. The compound of any one of embodiments 1-53 or 55, wherein L is a C₁ hydrocarbon chain, optionally substituted with halogen or —(CH₂)₀₋₄OR^{o}, wherein R^{o} is as defined above and described in classes and subclasses herein.
58. The compound of any one of embodiments 1-53, wherein L is —CH₂—, —C(O)—, —CF₂—, or —C(OH)H—.
59. The compound of any one of embodiments 1-53, wherein L is an optionally substituted C₂ hydrocarbon chain, wherein 1 methylene unit is optionally replaced with —NR^{z}— or —O—.
60. The compound of any one of embodiments 1-53 or 59, wherein L is an optionally substituted C₂ hydrocarbon chain, wherein the methylene unit connected to Cy^{A} is replaced with —NR^{z}— or —O—.
60. The compound of any one of embodiments 1-53 or 59-60, wherein L is selected from the group consisting of *—NHCH(Me)-, *—NHCH₂—, *—OCH₂—, and *—N(CH₃)CH₂—, wherein * represents the point of attachment to Cy^{A}.
61. The compound of any one of embodiments 1-53, wherein L comprises a two-atom spacer between Cy^{A} and

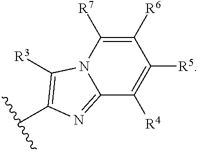

62. The compound of any one of embodiments 1-53, wherein L is an optionally substituted 5- to 6-membered saturated or partially unsaturated heterocyclene, having 1-3 heteroatoms independently selected from oxygen, nitrogen, and sulfur.
63. The compound of any one of embodiments 1-53 or 62, wherein L is an optionally substituted 5-membered saturated or partially unsaturated heterocyclene, having 1 heteroatom independently selected from oxygen, nitrogen, and sulfur.
64. The compound of any one of embodiments 1-53 or 62-63, wherein L is

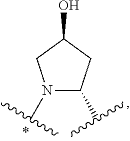 [SMILES: *[C@H]1C[C@H](O)CN1*]

wherein * represents the point of attachment to Cy^{A}.
65. The compound of any one of the preceding embodiments, wherein optional substituents on L are independently selected from halogen, —(CH₂)₀₋₄R^{o}, and —(CH₂)₀₋₄OR^{o}, wherein each R^{o} is independently as defined above and described in classes and subclasses herein.
66. The compound of any one of the preceding embodiments, wherein L′ is an optionally substituted C₁₋₄ hydrocarbon chain, wherein 1 to 3 methylene units are optionally and independently replaced with —O—, —C(O)—, —NR^{z}—, SO₂—, —S(NH)(O)—, or cyclopropylene.
67. The compound of any one of the preceding embodiments, wherein L′ is an optionally substituted C₁ hydrocarbon chain, wherein the 1 methylene unit is optionally replaced with —NR^{z}—.
68. The compound of any one of the preceding embodiments, wherein L′ is —CH₂— or —NH₂—.
69. The compound of any one of embodiments 1-66, wherein L′ is an optionally substituted C₂ hydrocarbon chain, wherein 1 to 2 methylene units are optionally and independently replaced with —O—, —C(O)—, or —NR^{z}—.
70. The compound of any one of embodiments 1-66 or 69, wherein L′ is —CH₂CH₂—,

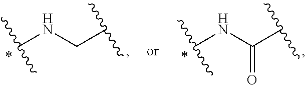

wherein * represents the point of attachment to Cy^{A}.
71. The compound of any one of embodiments 1-66, wherein L′ is an optionally substituted C₃ hydrocarbon chain, wherein 1 to 2 methylene units are optionally and independently replaced with —O—, —C(O)—, or —NR^{z}—.
72. The compound of any one of embodiments 1-66 or 71, wherein L′ is a C₃ hydrocarbon chain, optionally substituted with —(CH₂)₀₋₄R^{o} or —(CH₂)₀₋₄OR^{o}, wherein 1 to 2 methylene units are optionally and independently replaced with —O—, —C(O)—, or —NR^{z}—.
73. The compound of any one of embodiments 1-66 or 71-72, wherein L′ is an optionally substituted C₃ hydrocarbon chain, wherein 1 methylene unit is replaced with —C(O)—, and another methylene unit is replaced with —NR^{z}—.
74. The compound of any one of embodiments 1-66 or 71-72, wherein L′ is selected from the group consisting of:

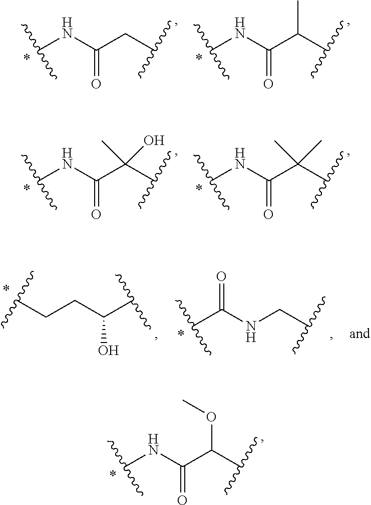

wherein * represents the point of attachment to Cy^{A}.
74. The compound of any one of embodiments 1-66, 71-72, or 74, wherein L′ is selected from the group consisting of:

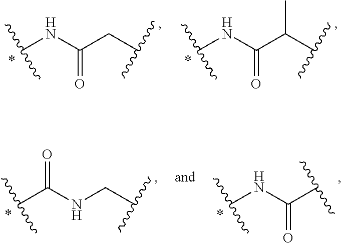

wherein * represents the point of attachment to Cy^{A}.
75. The compound of any one of embodiments 1-66, wherein L′ is an optionally substituted C₄ hydrocarbon chain, wherein 1 to 3 methylene units are optionally and independently replaced with —O—, —C(O)—, or —NR^{z}—.
76. The compound of any one of embodiments 1-66 or 75, wherein L′ is a C₄ hydrocarbon chain, optionally substituted with —(CH₂)₀₋₄R^{o} or ═NR₄, wherein 1 to 3 methylene units are optionally and independently replaced with —O—, —C(O)—, or —NR^{z}—.
77. The compound of any one of embodiments 1-66 or 75, wherein L′ is an optionally substituted C₄ hydrocarbon chain, wherein 1 methylene unit is replaced with —NR^{z}—, and 1 to 2 additional methylene units are optionally and independently replaced with —O—, —C(O)—, or —NR^{z}—.
78. The compound of any one of embodiments 1-66, 75, or 77, wherein L′ is an optionally substituted C₄ hydrocarbon chain, wherein 1 methylene unit is replaced with —NR^{z}—, 1 methylene unit is replaced with —C(O)—, and 1 methylene unity is optionally replaced with —O—, —C(O)—, or —NR^{z}—.
79. The compound of any one of embodiments 1-66 or 75, wherein L′ is selected from the group consisting of:

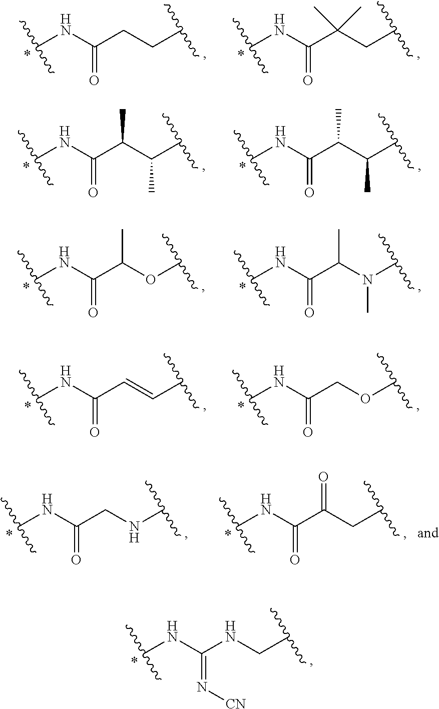

wherein * represents the point of attachment to Cy^{A}.
80. The compound of any one of embodiments 1-66, wherein L′ is an optionally substituted C₁₋₄ hydrocarbon chain, wherein 1 methylene unit of is replaced with cyclopropylene, and 1 to 2 additional methylene units are optionally and independently replaced with —O—, —C(O)—, —NR^{z}—, SO₂—, or —S(NH)(O)—.
81. The compound of any one of embodiments 1-66 or 80, wherein L′ is an optionally substituted C₁₋₄ hydrocarbon chain, wherein 1 methylene unit of is replaced with cyclopropylene, and 1 or 2 additional methylene units are independently replaced with —O—, —C(O)—, —NR^{z}—, SO₂—, or —S(NH)(O)—.
82. The compound of any one of embodiments 1-66 or 80-81, wherein L′ is selected from the group consisting of:

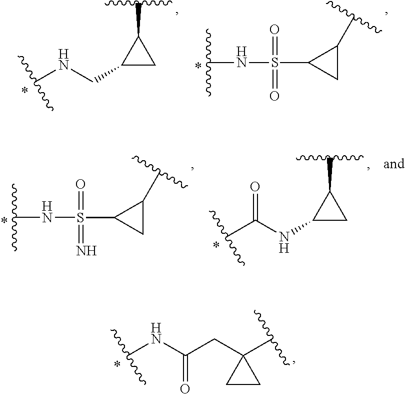 [SMILES: *NC[C@H]1C[C@@H]1* ; *NS(=O)(=O)C1CC1* ; *NS(=N)(=O)C1CC1* ; *C(=O)N[C@H]1C[C@@H]1* ; *NC(=O)CC1(*)CC1]

wherein * represents the point of attachment to Cy^{A}.
83. The compound of any one of embodiments 1-65, wherein L′ is a covalent bond.
84. The compound of any one of the preceding embodiments, wherein R³ is hydrogen.
85. The compound of any one of the preceding embodiments, wherein R⁴ is hydrogen.
86. The compound of any one of embodiments 1-84, wherein R⁴ is L^{C}-R^{C}.
87. The compound of any one of embodiments 1-84 or 86, wherein R⁴ is L^{C}-R^{C}, wherein L^{C} is a covalent bond, and R^{C} is Cy^{C}.
88. The compound of any one of embodiments 1-84 or 86-87, wherein R⁴ is L^{C}-R^{C}, wherein L^{C} is a covalent bond, and R^{C} is Cy^{C}, and Cy^{C} is 3- to 7-membered saturated or partially unsaturated monocyclic heterocyclyl.
88. The compound of any one of embodiments 1-84 or 86, R⁴ is L^{C}-R^{C}, wherein L^{C} is an optionally substituted C₁₋₆ hydrocarbon chain, and R^{C} is —OR or —OC(O)R.
89. The compound of any one of embodiments 1-84, wherein R⁴ is selected from the group consisting of:

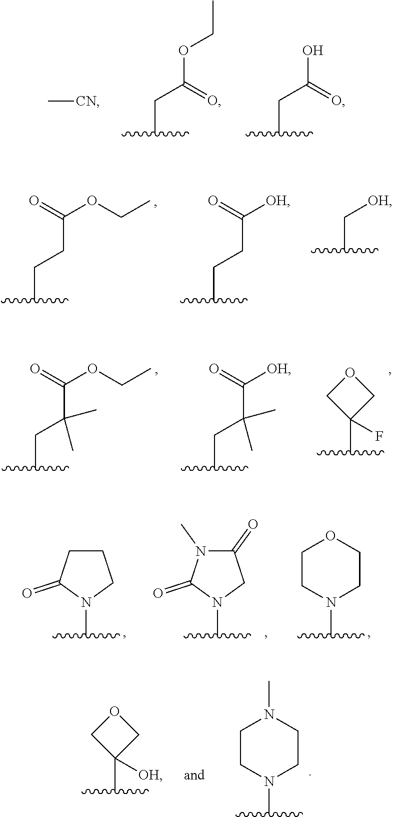

90. The compound of any one of the preceding embodiments, wherein R⁵ is hydrogen.
91. The compound of any one of embodiments 1-89, wherein R⁵ is cyclopropyl.
90. The compound of any one of the preceding embodiments, wherein R⁶ is hydrogen.
91. The compound of any one of embodiments 1-91, wherein R⁶ is cyclopropyl.
92. The compound of any one of the preceding embodiments, wherein R⁷ is hydrogen.
93. The compound of any one of embodiments 1-92, wherein the compound is of Formula (I-a), Formula (I-b), or Formula (I-c):

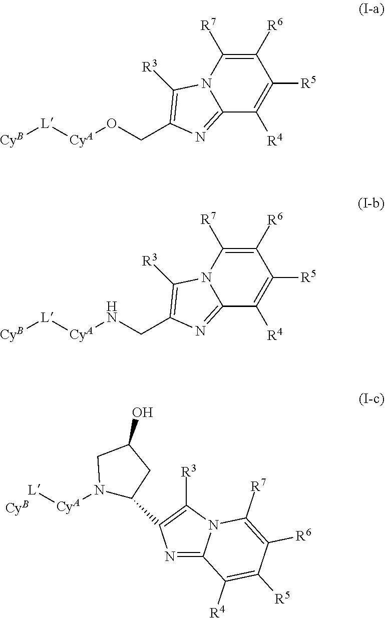

or a pharmaceutically acceptable salt thereof.
94. The compound of any one of embodiments 1-92, wherein the compound is of Formula (II), Formula (II-a), Formula (II-b), or Formula (II-c):

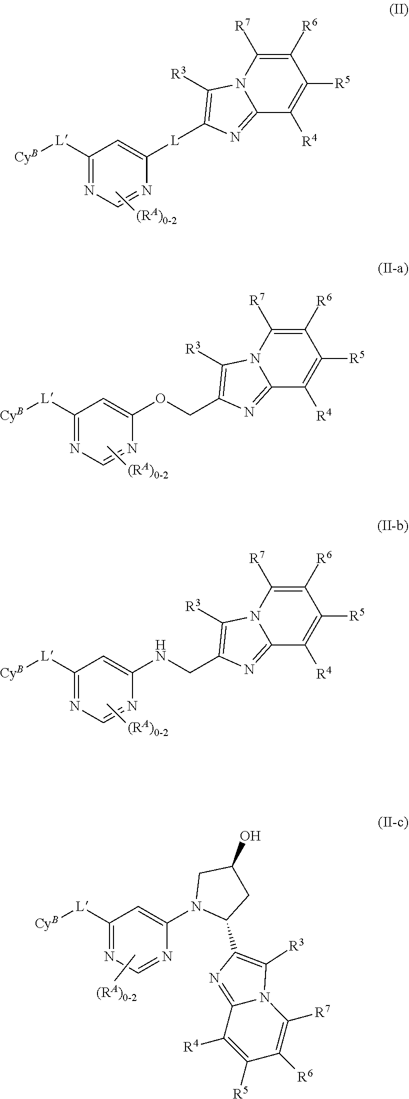

or a pharmaceutically acceptable salt thereof
95. The compound of any one of embodiments 1-92, wherein the compound is of Formula (III), Formula (III-a), Formula (III-b), or Formula (III-c):

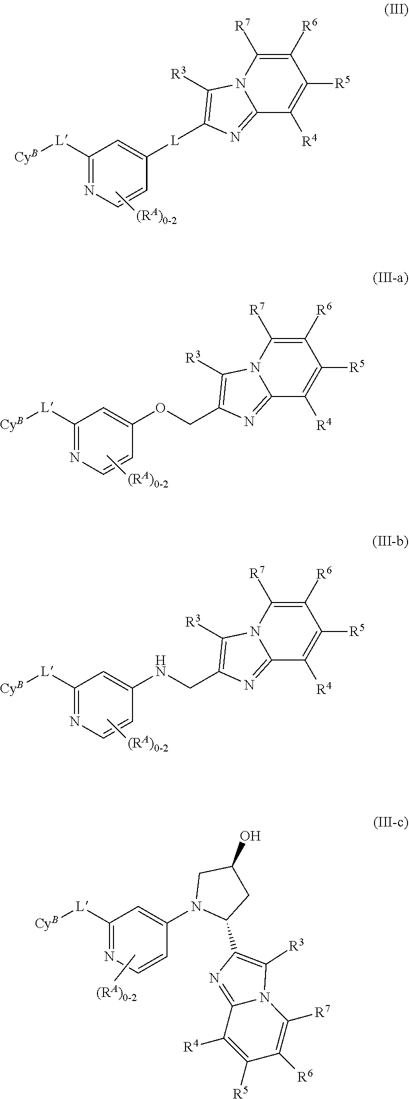

or a pharmaceutically acceptable salt thereof.
96. The compound of any one of embodiments 1-92, wherein the compound is of Formula (IV-a), Formula (IV-b), Formula (IV-c), or Formula (IV-d):

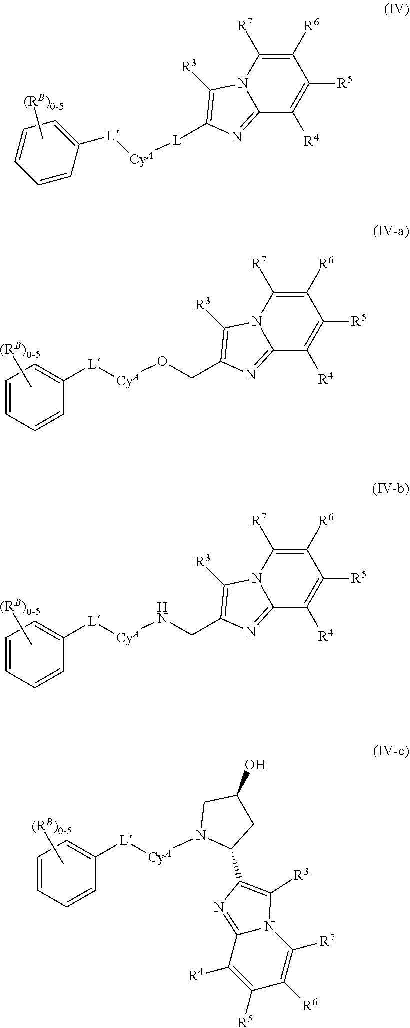

or a pharmaceutically acceptable salt thereof.
97. The compound of any one of embodiments 1-92, wherein the compound is of Formula (V), (V-a), Formula (V-b), or Formula (V-c):

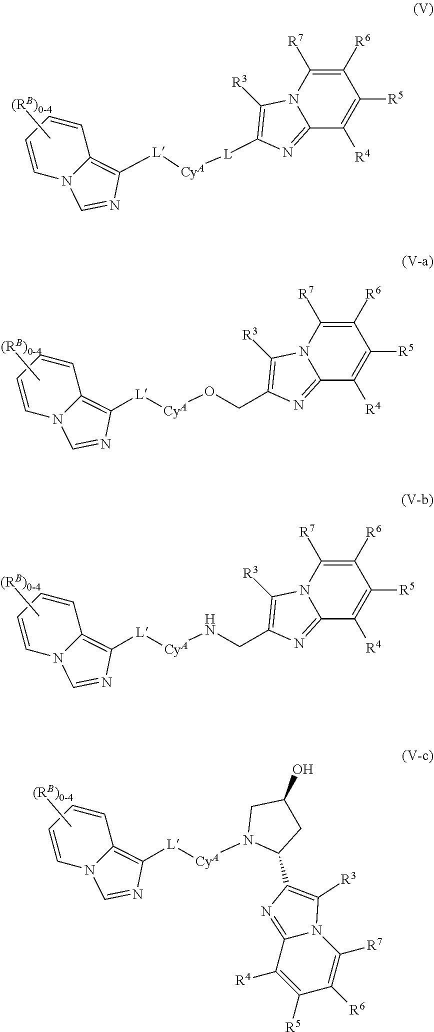

or a pharmaceutically acceptable salt thereof.
98. The compound of any one of embodiments 1-92, wherein the compound is of Formula (VI), (VI-a), Formula (VI-b), or Formula (VI-c):

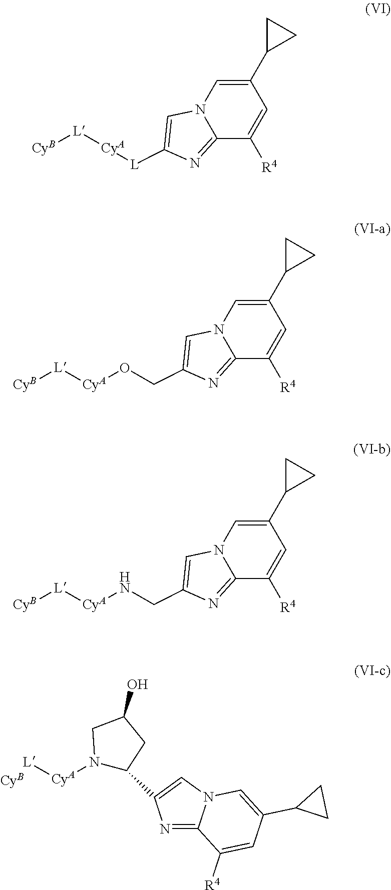

or a pharmaceutically acceptable salt thereof.
99. The compound of any one of embodiments 1-92, wherein the compound is of Formula (VII), (VII-a), Formula (VII-b), or Formula (VII-c):

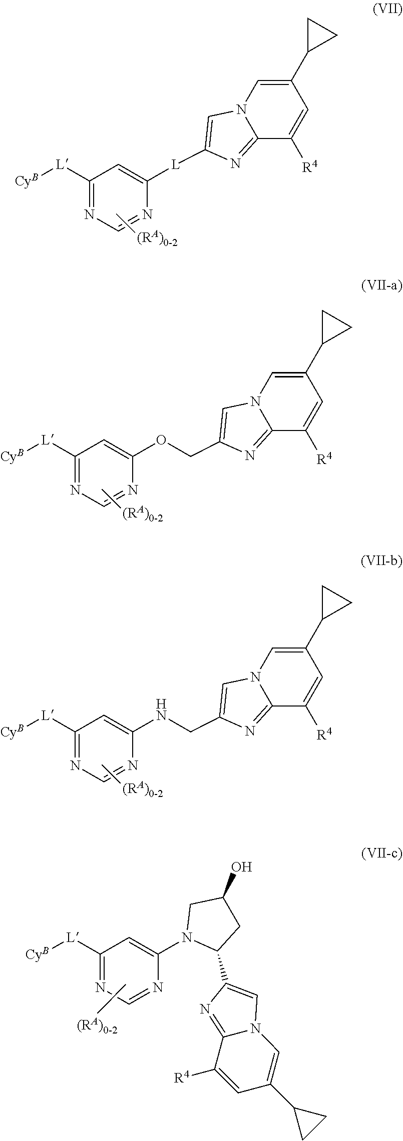

or a pharmaceutically acceptable salt thereof
100. The compound of any one of embodiments 1-92, wherein the compound is of Formula (VIII), (VIII-a), Formula (VIII-b), or Formula (VIII-c):

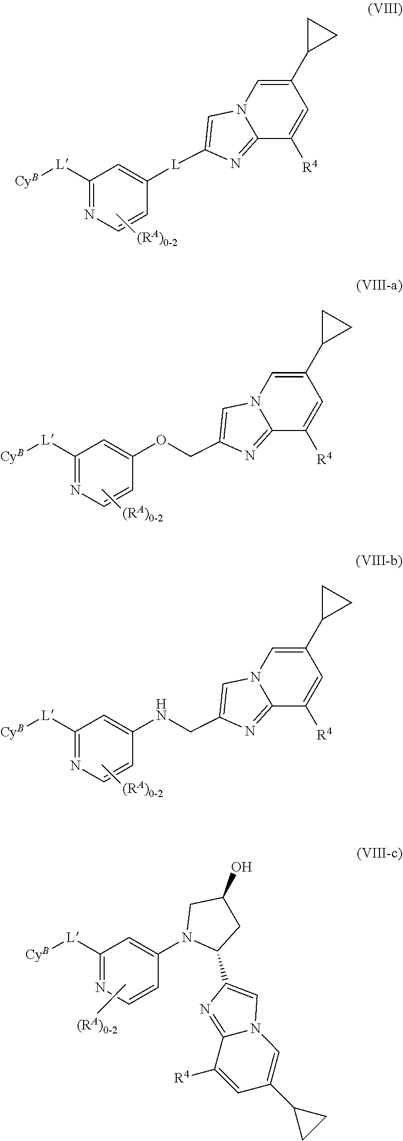

or a pharmaceutically acceptable salt thereof.
101. The compound of any one of the preceding embodiments, wherein the compound is selected from compounds I-1 through I-56, I-58, I-62 through I-152, or I-154 through I-161, or a pharmaceutically acceptable salt thereof.
102 A compound, or pharmaceutically acceptable salt thereof, selected from compounds I-57, I-59 through I-61, or I-53 or a pharmaceutically acceptable salt thereof.
103. A pharmaceutical composition comprising a compound of any one of the preceding embodiments.
104. The pharmaceutical composition comprising a compound of any one of the preceding embodiments, further comprising a pharmaceutically acceptable excipient.
105. The composition of embodiment 103 or 104, wherein the composition is suitable for oral administration.
106. A method of treating a plasma kallikrein-mediated disease or disorder using a compound or composition of any one of the preceding embodiments.
107. The method of embodiment 106, wherein the disease or disorder is hereditary angioedema.
108. The method of embodiment 106, wherein the disease or disorder is diabetic macular edema.
109. A method of treating hereditary angioedema comprising administering to a patient in need thereof a compound or composition of any one of the preceding embodiments.
110. A method of treating diabetic macular edema comprising administering to a patient in need thereof a compound or composition of any one of the preceding embodiments.
111. The method of any one of embodiments 107 or 109, wherein administration of the compound partially or completely inhibits, delays onset of, reduces severity of, and/or reduces incidence of one or more symptoms, features, and/or causes of a hereditary angioedema.
112. The method of embodiment 111, wherein the compound is administered orally.

1. A compound of formula I: 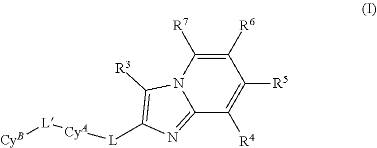
or a pharmaceutically acceptable salt thereof,
wherein:
Cy^{A} is a 4-membered monocyclic carbocyclene, a 3- to 7-membered saturated or partially unsaturated monocyclic heterocyclene having 1-3 heteroatoms selected from oxygen, nitrogen, or sulfur, phenylene, a 5- to 6-membered monocyclic heteroarylene having 1-3 heteroatoms independently selected from oxygen, nitrogen, and sulfur, a 7- to 10-membered saturated or partially unsaturated bicyclic heterocyclene having 1-4 heteroatoms selected from oxygen, nitrogen, or sulfur, or an 8- to 12-membered bicyclic heteroarylene having 1-4 heteroatoms independently selected from oxygen, nitrogen, and sulfur, wherein Cy^{A} is substituted with 0-4-R^{A} groups;
each R^{A} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, 3- to 7-membered saturated or partially unsaturated monocyclic carbocyclyl, 3- to 7-membered saturated or partially unsaturated monocyclic heterocyclyl having 1-2 heteroatoms selected from oxygen, nitrogen, or sulfur, or a 5- to 6-membered monocyclic heteroaryl having 1-4 heteroatoms independently selected from oxygen, nitrogen or sulfur;
each R is independently hydrogen or an optionally substituted C₁₋₆ aliphatic group;
Cy^{B} is selected from phenyl, 8- to 10-membered bicyclic aryl, 7- to 10-membered saturated or partially unsaturated bicyclic carbocycyl, a 5- to 6-membered heteroaryl having 1-3 heteroatoms independently selected from oxygen, nitrogen, and sulfur, a 7- to 10-membered saturated or partially unsaturated bicyclic heterocycyl having 1-3 heteroatoms selected from oxygen, nitrogen, or sulfur, or a 7- to 10-membered heteroaryl having 1-4 heteroatoms independently selected from oxygen, nitrogen, and sulfur, wherein Cy^{B} is substituted with 0-5-R^{B} groups; or
each R^{B} is independently selected from oxo, halogen, —CN, —C(O)R, —C(O)₂R, —C(O)N(R)₂, —C(NR)NR₂, —C(NR)NROR, —C(NR)NRC(O)OR, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, a 3- to 7-membered saturated or partially unsaturated monocyclic heterocyclyl having 1-2 heteroatoms selected from oxygen, nitrogen, or sulfur, or a 5- to 6-membered heteroaryl having 1-4 heteroatoms independently selected from oxygen, nitrogen, and sulfur;
L′ is a covalent bond or an optionally substituted C₁₋₄ hydrocarbon chain, wherein 1 to 3 methylene units are optionally and independently replaced with —O—, —C(O)—, —NR^{z}—, —S—, —SO—, SO₂—, —S(NH)(O)—, or cyclopropylene;
each R^{z} is independently selected from hydrogen, —(CH₂)₀₋₃OR, —(CH₂)₀₋₃C(O)OR, or an optionally substituted C₁₋₆ aliphatic group;
L is an optionally substituted C₁₋₂ hydrocarbon chain, wherein 1 methylene unit is optionally and independently replaced with —C(O)—, —O—, —NR^{z}—, —S—, —SO—, or —SO₂—; or
L is an optionally substituted 5- to 6-membered saturated or partially unsaturated heterocyclene, having 1-3 heteroatoms independently selected from oxygen, nitrogen, and sulfur;
each R³, R⁴, R⁵, R⁶, and R⁷ is independently selected from hydrogen or -L^{C}-R^{C}, wherein
each L^{C} is independently selected from a covalent bond or an optionally substituted C₁₋₆ hydrocarbon chain, wherein 1 to 3 methylene units are optionally and independently replaced with —O— or —NR—;
each R^{C} is independently selected from halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, Cy^{C}, or an optionally substituted C₁₋₆ aliphatic; and
each Cy^{C} is an optionally substituted ring independently selected from a 3- to 7-membered saturated or partially unsaturated monocyclic carbocyclyl, a 3- to 7-membered saturated or partially unsaturated monocyclic heterocyclyl having 1-2 heteroatoms selected from oxygen, nitrogen, or sulfur, a 5- to 6-membered monocyclic heteroaryl having 1-3 heteroatoms independently selected from oxygen, nitrogen, and sulfur, phenyl, a 6- to 12-membered saturated or partially unsaturated fused bicyclic heterocyclyl having 1-3 heteroatoms independently selected from oxygen, nitrogen, or sulfur, a bridged bicycle, or a 6- to 12-membered saturated or partially unsaturated bicyclic spiroheterocyclyl having 1-3 heteroatoms independently selected from oxygen, nitrogen, or sulfur.

2. The compound of claim 1, wherein Cy^{A} is a 4-membered monocyclic carbocyclene, a 3- to 7-membered saturated or partially unsaturated monocyclic heterocyclene having 1-3 heteroatoms selected from oxygen, nitrogen, or sulfur, a 5- to 6-membered monocyclic heteroarylene having 1-3 heteroatoms independently selected from oxygen, nitrogen, and sulfur, a 7- to 10-membered saturated or partially unsaturated bicyclic heterocyclene having 1-4 heteroatoms selected from oxygen, nitrogen, or sulfur, or a 7- to 10-membered bicyclic heteroarylene having 1-4 heteroatoms independently selected from oxygen, nitrogen, and sulfur, wherein Cy^{A} is substituted with 0-4-R^{A} groups.

3. The compound of claim 1, wherein Cy^{A} is selected from the group consisting of: 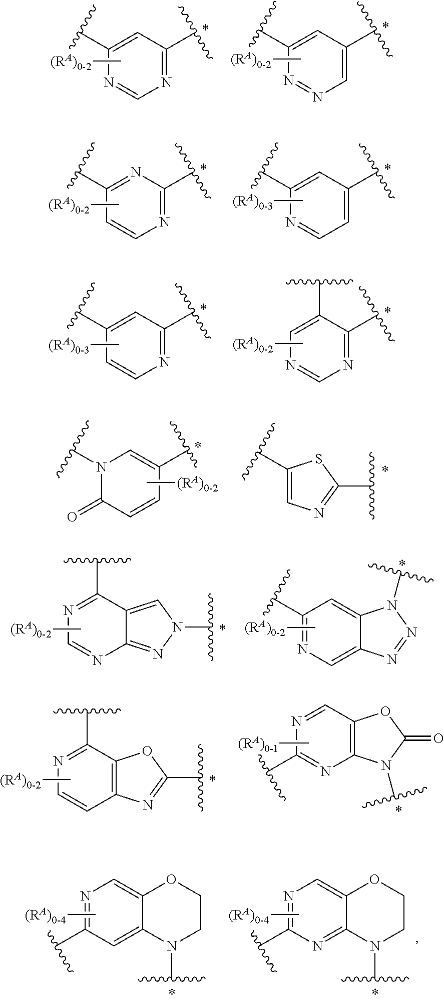
wherein * represents the point of attachment to L.

4. The compound of claim 1, wherein each R^{A} is independently selected from oxo, —C(O)R, —C(O)₂R, —OR, or an optionally substituted group selected from C₁₋₆ aliphatic or a 5- to 6-membered monocyclic heteroaryl having 1-4 heteroatoms independently selected from oxygen, nitrogen or sulfur.

5. The compound of claim 1,
wherein Cy^{B} is selected from the group consisting of:

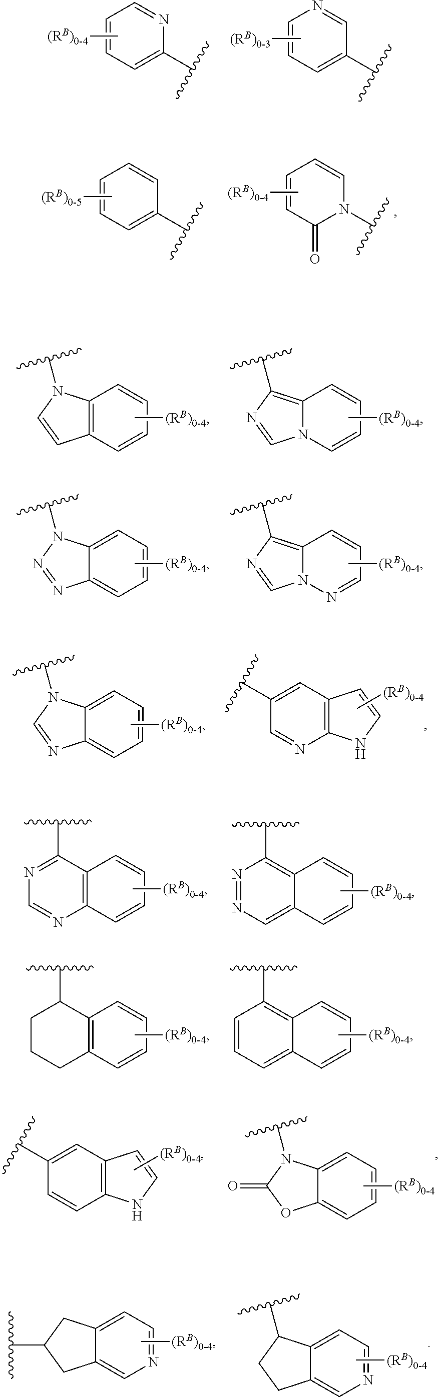

6. The compound of claim 1, wherein each R^{B} is independently selected from oxo, halogen, —CN, —C(O)N(R)₂, —C(NR)NR₂, —C(NR)NROR, —C(NR)NRC(O)OR, —N(R)₂, —OR, or an optionally substituted group selected from C₁₋₆ aliphatic, or a 5- to 6-membered heteroaryl having 1-4 heteroatoms independently selected from oxygen, nitrogen, and sulfur.

7. The compound of claim 1, wherein L is an optionally substituted C₁₋₂ hydrocarbon chain, wherein 1 methylene unit is optionally replaced with —C(O)—, —O—, —NR^{z}—; or L is an optionally substituted 5- to 6-membered saturated or partially unsaturated heterocyclene, having 1-3 heteroatoms independently selected from oxygen, nitrogen, and sulfur.

8. The compound of claim 1, wherein L is —CH₂—, —C(O)—, —CF₂—, or —C(OH)H—, *—NHCH(Me)-, *—NHCH₂—, *—OCH₂—, or *—N(CH₃)CH₂—, wherein * represents the point of attachment to Cy^{A}.

9. The compound of claim 1, wherein L′ is an optionally substituted C₁₋₄ hydrocarbon chain, wherein 1 to 3 methylene units are optionally and independently replaced with —O—, —C(O)—, —NR^{z}—, SO₂—, —S(NH)(O)—, or cyclopropylene.

10. The compound of claim 1, wherein L′ is selected from the group consisting of: 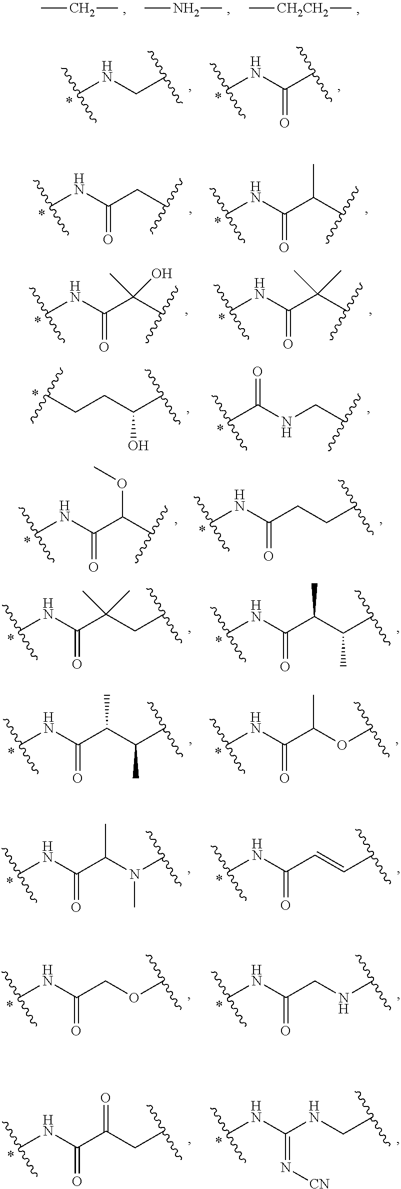 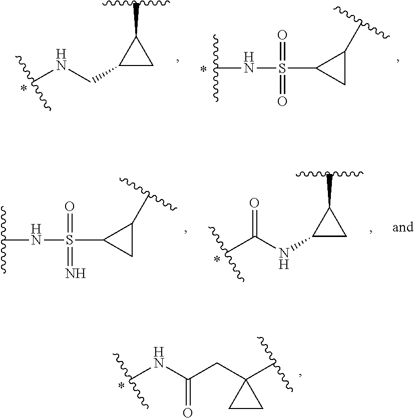 [SMILES: *NC[C@H]1C[C@@H]1* ; *NS(=O)(=O)C1CC1* ; *NS(=N)(=O)C1CC1* ; *C(=O)N[C@H]1C[C@@H]1* ; *NC(=O)CC1(*)CC1]
wherein * represents the point of attachment to Cy^{A}.

11. The compound of claim 1, wherein L′ is a covalent bond.

12. The compound of claim 1, wherein R³ is hydrogen.

13. The compound of claim 1,
wherein R⁴ is selected from the group consisting of:
hydrogen, —CN,

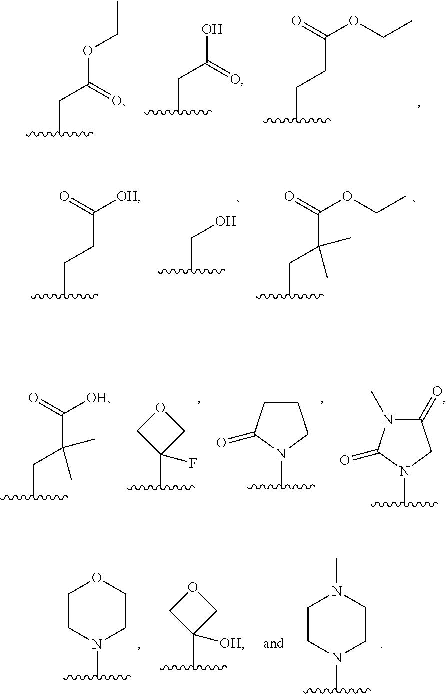

14. The compound of claim 1, wherein R⁵ is hydrogen or cyclopropyl.

15. The compound of claim 1, wherein R⁶ is hydrogen or cyclopropyl.

16. The compound of ay claim 1, wherein R⁷ is hydrogen.

17. The compound of claim 1, wherein the compound is of Formula (I-a), Formula (I-b), or Formula (I-c): 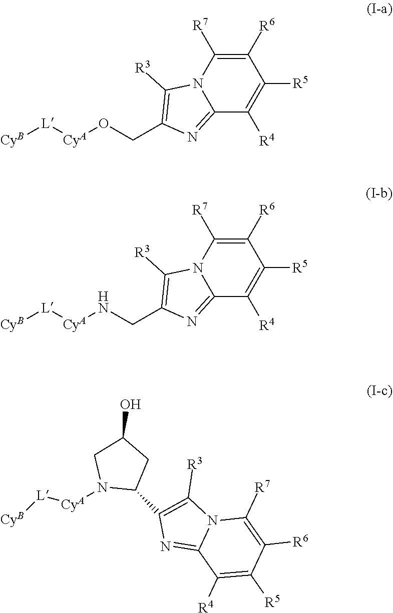
or a pharmaceutically acceptable salt thereof.

18. The compound of claim 1, wherein the compound is of Formula (II), Formula (II-a), Formula (II-b), or Formula (II-c): 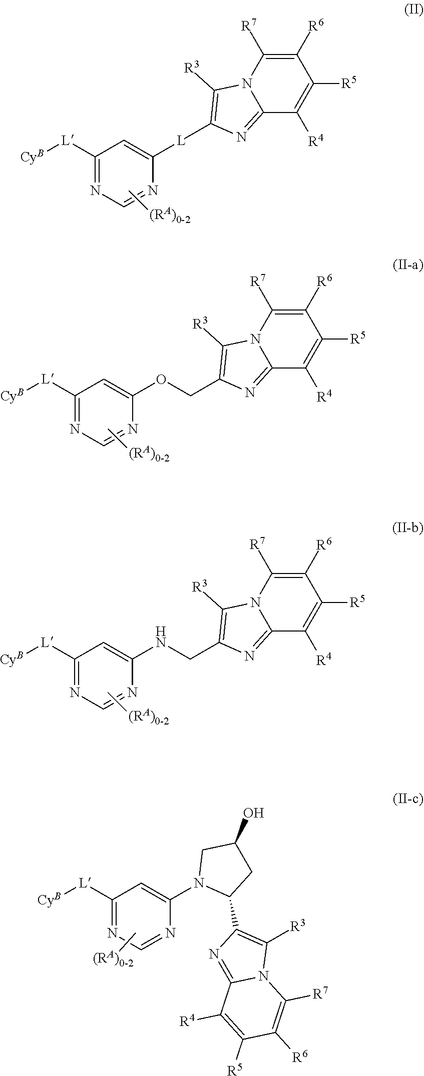
or a pharmaceutically acceptable salt thereof.

19. The compound of claim 1, wherein the compound is of Formula (III), Formula (III-a), Formula (III-b), or Formula (III-c): 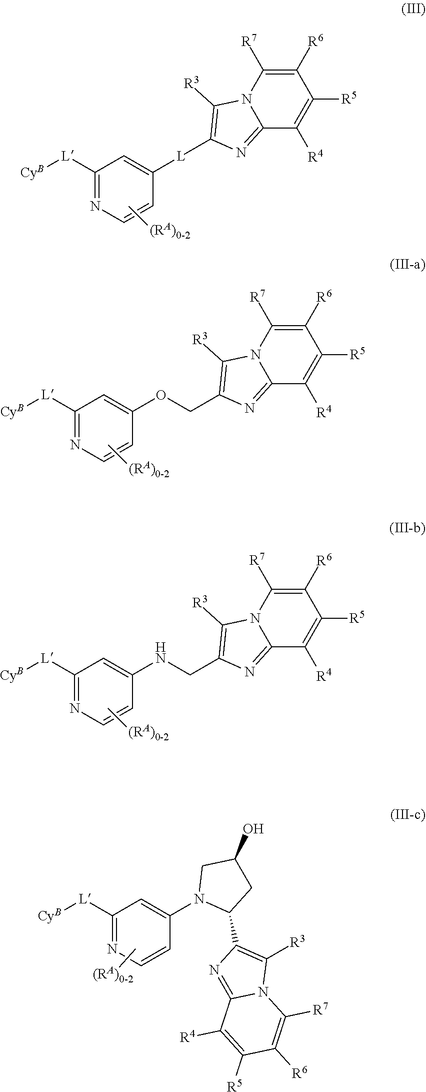
or a pharmaceutically acceptable salt thereof.

20. The compound of claim 1, wherein the compound is of Formula (IV-a), Formula (IV-b), Formula (IV-c), or Formula (IV-d): 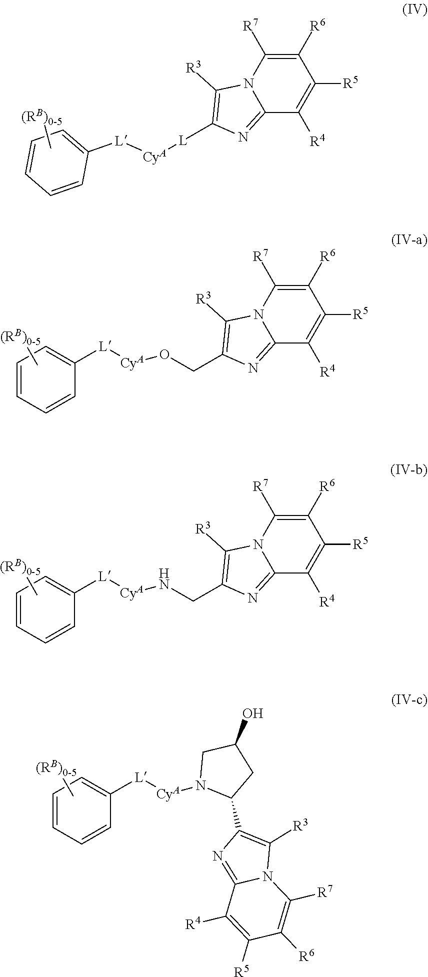
or a pharmaceutically acceptable salt thereof.

21. The compound of claim 1, wherein the compound is of Formula (V), (V-a), Formula (V-b), or Formula (V-c): 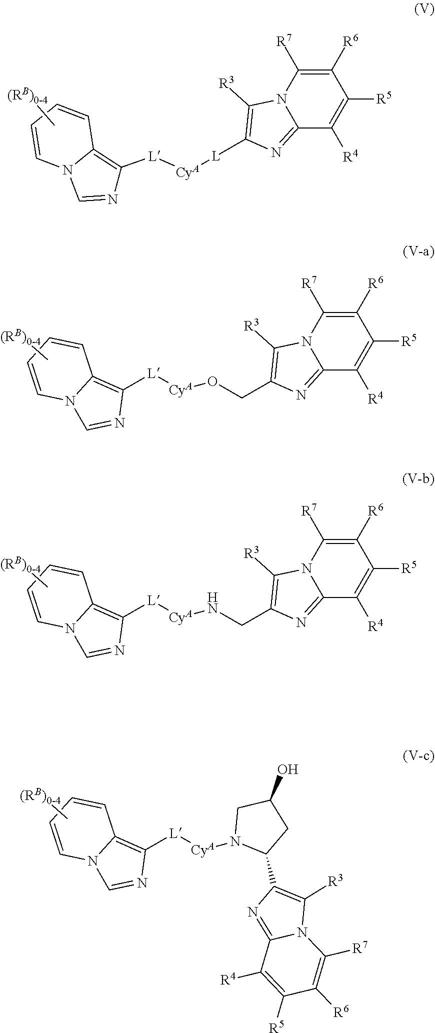
or a pharmaceutically acceptable salt thereof.

22. The compound of claim 1, wherein the compound is of Formula (VI), (VI-a), Formula (VI-b), or Formula (VI-c): 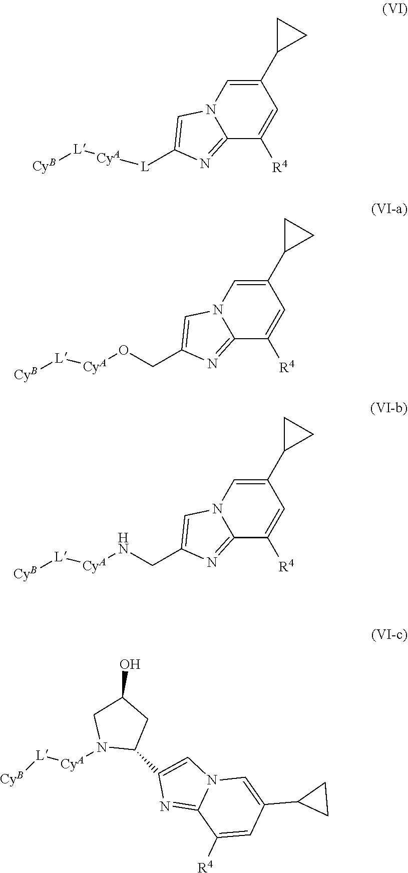
or a pharmaceutically acceptable salt thereof.

23. The compound of claim 1, wherein the compound is of Formula (VII), (VII-a), Formula (VII-b), or Formula (VII-c): 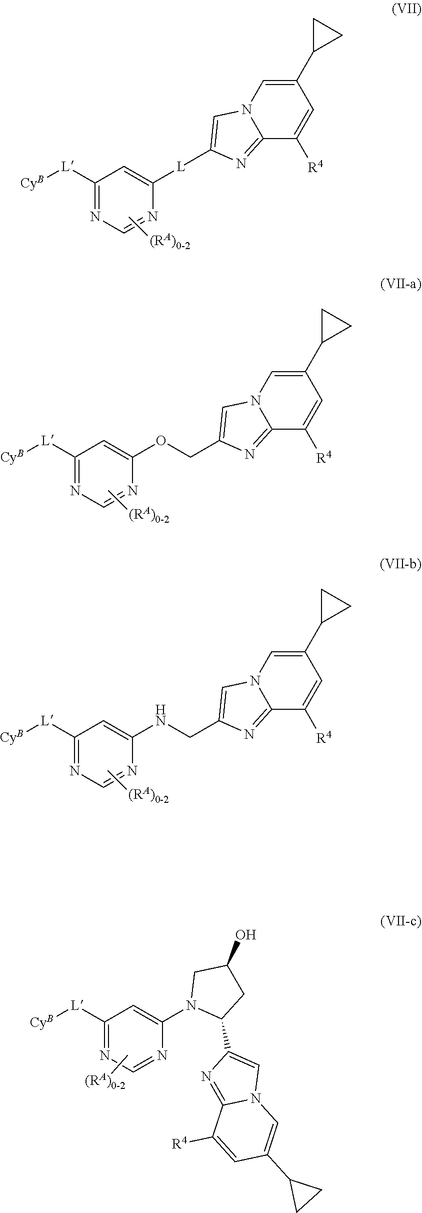
or a pharmaceutically acceptable salt thereof.

24. The compound of claim 1, wherein the compound is of Formula (VIII), (VIII-a), Formula (VIII-b), or Formula (VIII-c): 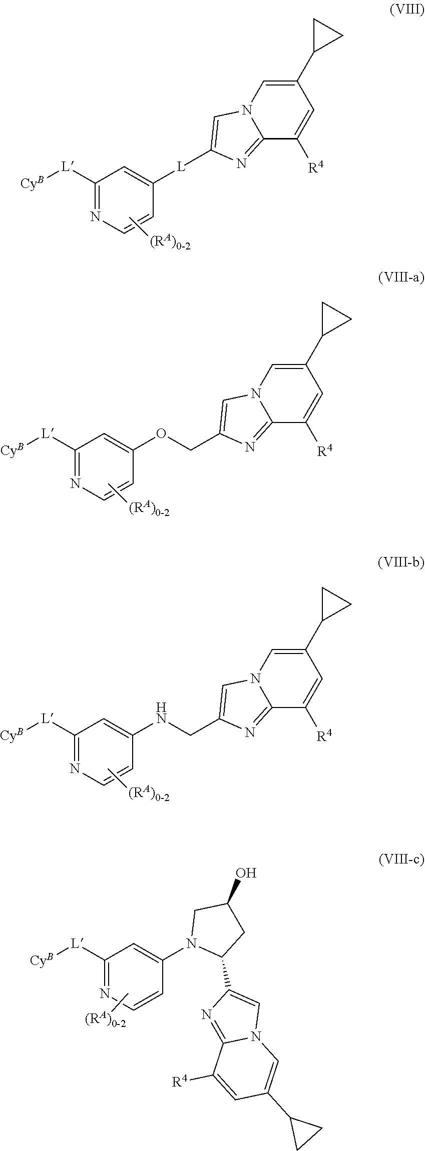
or a pharmaceutically acceptable salt thereof.

25. The compound of claim 1, wherein the compound is selected from compounds I-1 through I-56, I-58, I-62 through I-152, or I-154 through I-161, or a pharmaceutically acceptable salt thereof.

26. A compound, or pharmaceutically acceptable salt thereof, selected from compounds I-57, I-59 through I-61, or I-53 or a pharmaceutically acceptable salt thereof.

27. A pharmaceutical composition comprising a compound of claim 1.

28. A method of treating a plasma kallikrein-mediated disease or disorder using a compound of claim 1.

29. The method of claim 28, wherein the disease or disorder is hereditary angioedema or diabetic macular edema.
